(19) United States
(12) Patent Application Publication
(10) Pub. No.: US 2026/0143897 A1
(43) Pub. Date: May 21, 2026

(54) COMPOUND FOR ORGANIC OPTOELECTRONIC DEVICE, COMPOSITION FOR ORGANIC OPTOELECTRONIC DEVICE, ORGANIC OPTOELECTRONIC DEVICE, AND DISPLAY DEVICE

(71) Applicant: SAMSUNG SDI CO., LTD., Yongin-si, Gyeonggi-do (KR)

(72) Inventors: Seonyeong GWAK, Suwon-si, Gyeonggi-do (KR); Yoonman LEE, Suwon-si, Gyeonggi-do (KR); Suheon IM, Suwon-si, Gyeonggi-do (KR); Seungjae LEE, Suwon-si, Gyeonggi-do (KR); Minseok SEO, Suwon-si, Gyeonggi-do (KR); Mijin LEE, Suwon-si, Gyeonggi-do (KR); Wook KIM, Suwon-si, Gyeonggi-do (KR); Sung-Hyun JUNG, Suwon-si, Gyeonggi-do (KR); Hyung Sun KIM, Suwon-si, Gyeonggi-do (KR); Ho Kuk JUNG, Suwon-si, Gyeonggi-do (KR); Hanill LEE, Suwon-si, Gyeonggi-do (KR)

(21) Appl. No.: 18/719,081

(22) PCT Filed: Apr. 10, 2023
(86) PCT No.: PCT/KR2023/004822

§ 371 Date: Jun. 12, 2024

(30) Foreign Application Priority Data
Apr. 11, 2022 (KR) 10-2022-0044723

(51) Int. Cl. H10K 50/11; C07B 59/00; C07D 487/04; H10K 85/30; H10K 85/40; H10K 85/60

(52) U.S. Cl. CPC H10K 50/11; C07B 59/004; C07D 487/04; H10K 85/342; H10K 85/40; H10K 85/622; H10K 85/654; H10K 85/6572; H10K 85/6574; H10K 85/6576; C07B 2200/05

(57) ABSTRACT

Provided are a compound for an organic optoelectronic device represented by a combination of Chemical Formula 1 and Chemical Formula 2, a composition for an organic optoelectronic device including the same, an organic optoelectronic device, and a display device. The contents of Chemical Formula 1 and Chemical Formula 2 are as defined in the specification.

### TECHNICAL FIELD

Compounds for organic optoelectronic devices, compositions for organic optoelectronic devices, organic optoelectronic devices, and display devices are disclosed.

### BACKGROUND ART

An organic optoelectronic device (organic optoelectronic diode) is a device capable of converting electrical energy and optical energy to each other.

Organic optoelectronic devices may be largely divided into two types according to a principle of operation. One is a photoelectric device that generates electrical energy by separating excitons formed by light energy into electrons and holes, and transferring the electrons and holes to different electrodes, respectively and the other is light emitting device that generates light energy from electrical energy by supplying voltage or current to the electrodes.

Examples of the organic optoelectronic device include an organic photoelectric device, an organic light emitting diode, an organic solar cell, and an organic photoconductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode is greatly influenced by an organic material between electrodes.

### DISCLOSURE

### Technical Problem

An embodiment provides a compound for an organic optoelectronic device capable of implementing low-driving, high efficiency, and long life-span organic optoelectronic device.

Another embodiment provides a composition for an organic optoelectronic device capable of implementing a highly efficient and long life-span organic optoelectronic device.

Another embodiment provides an organic optoelectronic device containing the compound.

Another embodiment provides a display device including the organic optoelectronic device.

### Technical Solution

According to an embodiment, a compound for an organic optoelectronic device represented by a combination of Chemical Formula 1 and Chemical Formula 2 is provided.

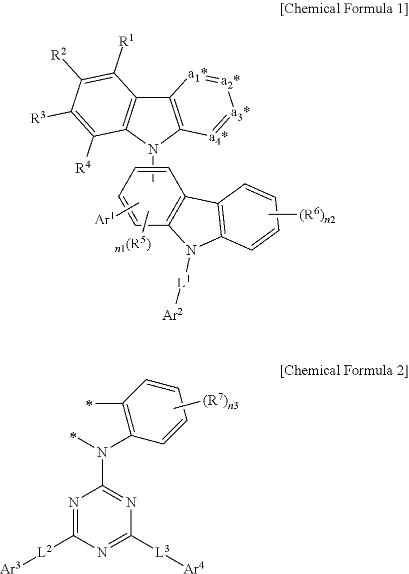

In Chemical Formulas 1 and 2,
Ar¹ is a substituted or unsubstituted C6 to C30 aryl group, a substituted or unsubstituted dibenzofuranyl group, a substituted or unsubstituted dibenzothiophenyl group, or a substituted or unsubstituted carbazolyl group,
Ar² to Ar⁴ are each independently a substituted or unsubstituted C6 to C20 aryl group or a substituted or unsubstituted C2 to C30 heterocyclic group,
among a₁* to a₄* in Chemical Formula 1, two adjacent ones are each linking carbon (C) linked to * in Chemical Formula 2,
among a₁* to a₄* in Chemical Formula 1, the remaining two not linked to * in Chemical Formula 2 are each independently C-L^{a}-R^{a},
L^{a}, and L¹ to L³ are each independently a single bond or a substituted or unsubstituted C6 to C20 arylene group,
R^{a} and R¹ to R⁷ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
n1 is an integer of 1 or 2, and
n2 and n3 are each independently one of integers of 1 to 4.

According to another embodiment, a composition for an organic optoelectronic device including a first compound and a second compound is provided.

The first compound may be the same as described above, and the second compound may be a compound for an organic optoelectronic device represented by Chemical Formula 3; or a compound for an organic optoelectronic device represented by a combination of Chemical Formula 4 and Chemical Formula 5.

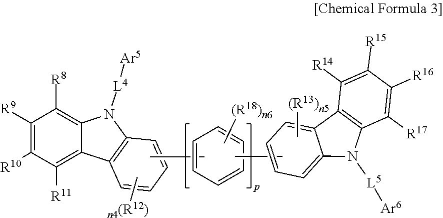

In Chemical Formula 3,
Ar⁵ and Ar⁶ are each independently a substituted or unsubstituted C6 to C20 aryl group or a substituted or unsubstituted C2 to C30 heterocyclic group,
L⁴ and L⁵ are each independently a single bond or a substituted or unsubstituted C6 to C20 arylene group,
R⁸ to R¹⁸ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
n4 and n5 are each independently one of integers of 1 to 3,
n6 is one of integers of 1 to 4, and
p is one of integers of 0 to 2;

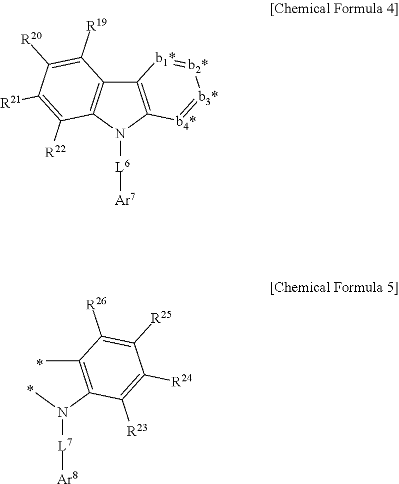

wherein, in Chemical Formulas 4 and 5,
Ar⁷ and Ar⁸ are each independently a substituted or unsubstituted C6 to C20 aryl group or a substituted or unsubstituted C2 to C30 heterocyclic group,
b₁* to b₄* in Chemical Formula 4 are each independently a linking carbon (C) or C-L^{b}-R^{b},
among b₁* to b₄* in Chemical Formula 4, the two adjacent ones are linked to * in Chemical Formula 5,
L^{b}, L⁶, and L⁷ are each independently a single bond or a substituted or unsubstituted C6 to C20 arylene group, and
R^{b} and R¹⁹ to R²⁶ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

According to another embodiment, an organic optoelectronic device includes an anode and a cathode facing each other, and at least one organic layer between the anode and the cathode, and the organic layer includes the compound for an organic optoelectronic device or the composition for an organic optoelectronic device.

According to another embodiment, a display device including the organic optoelectronic device is provided.

### Advantageous Effects

An organic optoelectronic device having low-driving, high efficiency, and long life-span may be realized.

### DESCRIPTION OF THE DRAWINGS

FIG. 1 is a cross-sectional view showing an organic light emitting diode according to an embodiment.

### DESCRIPTION OF SYMBOLS

100: organic light emitting diode
105: organic layer
110: cathode
120: anode
130: light emitting layer
140: hole transport region
150: electron transport region

### MODE FOR INVENTION

Hereinafter, embodiments of the present invention are described in detail. However, these embodiments are exemplary, the present invention is not limited thereto and the present invention is defined by the scope of claims.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example of the present invention, the “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In specific example of the present invention, the “substituted” refers to replacement of at least one hydrogen of a substituent or a compound by deuterium, a C1 to C20 alkyl group, a C6 to C30 aryl group, or a cyano group. In specific example of the present invention, the “substituted” refers to replacement of at least one hydrogen of a substituent or a compound by deuterium, a C1 to C5 alkyl group, a C6 to C18 aryl group, or a cyano group. In specific example of the present invention, the “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and all elements of the hydrocarbon aromatic moiety have p-orbitals which form conjugation, for example a phenyl group, a naphthyl group, and the like, two or more hydrocarbon aromatic moieties may be linked by a sigma bond and may be, for example a biphenyl group, a terphenyl group, a quarterphenyl group, and the like, and two or more hydrocarbon aromatic moieties are fused directly or indirectly to provide a non-aromatic fused ring, for example a fluorenyl group.

The aryl group may include a monocyclic, polycyclic, or fused ring polycyclic (i.e., rings sharing adjacent pairs of carbon atoms) functional group.

As used herein, “a heterocyclic group” is a generic concept of a heteroaryl group, and may include at least one heteroatom selected from N, O, S, P, and Si instead of carbon (C) in a cyclic compound such as aryl group, a cycloalkyl group, a fused ring thereof, or a combination thereof. When the heterocyclic group is a fused ring, the entire ring or each ring of the heterocyclic group may include one or more heteroatoms.

For example, “a heteroaryl group” may refer to aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, a substituted or unsubstituted furanyl group, or a combination thereof, but is not limited thereto.

More specifically, a substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzothiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, or a substituted or unsubstituted dibenzothiophenyl group, or a combination thereof, but is not limited thereto.

“Unsubstituted” refers to non-replacement of a hydrogen atom by another substituent and remaining of the hydrogen atom.

In the present specification, “hydrogen substitution (—H)” may include “deuterium substitution (-D)” or “tritium substitution (-T).”

As used herein, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, a compound for an organic optoelectronic device according to an embodiment is described.

The compound for an organic optoelectronic device according to an embodiment is represented by a combination of Chemical Formula 1 and Chemical Formula 2.

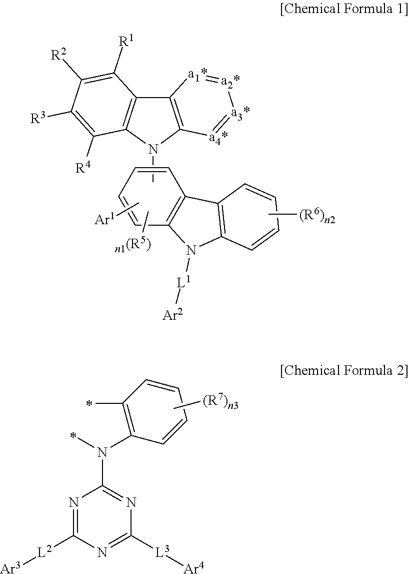

In Chemical Formulas 1 and 2,
Ar¹ is a substituted or unsubstituted C6 to C30 aryl group, a substituted or unsubstituted dibenzofuranyl group, a substituted or unsubstituted dibenzothiophenyl group, or a substituted or unsubstituted carbazolyl group,
Ar² to Ar⁴ are each independently a substituted or unsubstituted C6 to C20 aryl group or a substituted or unsubstituted C2 to C30 heterocyclic group,
among a₁* to a₄* in Chemical Formula 1, two adjacent ones are each linking carbon (C) linked to * in Chemical Formula 2,
among a₁* to a₄* in Chemical Formula 1, the remaining two not linked to * in Chemical Formula 2 are each independently C-L^{a}-R^{a},
L^{a}, and L¹ to L³ are each independently a single bond or a substituted or unsubstituted C6 to C20 arylene group,
R^{a} and R¹ to R⁷ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
n1 is an integer of 1 or 2, and
n2 and n3 are each independently one of integers of 1 to 4.

The compound represented by the combination of Chemical Formula 1 and Chemical Formula 2 has a structure including a triazine directly substituted and a directly substituted carbazolyl group in the N-direction in the indolocarbazole skeleton.

When carbazole is directly substituted in the N-direction of the indolocarbazole skeleton, HT properties are strengthened compared to structures in which the indolocarbazole skeleton is substituted in the phenyl direction, resulting in better hole flow. As a result, the electron/hole injection ability is improved and the driving voltage may be lowered.

In particular, by substituting the carbazolyl group directly substituted in the N-direction of the indolocarbazole skeleton, with an additional substituent, deterioration and decomposition may be to minimized by reducing the deposition temperature as a structure with steric hindrance, and thus, the life-span characteristics can be further improved.

As an example, Chemical Formula 1 may be represented by any one of Chemical Formula 1-1 to Chemical Formula 1-4.

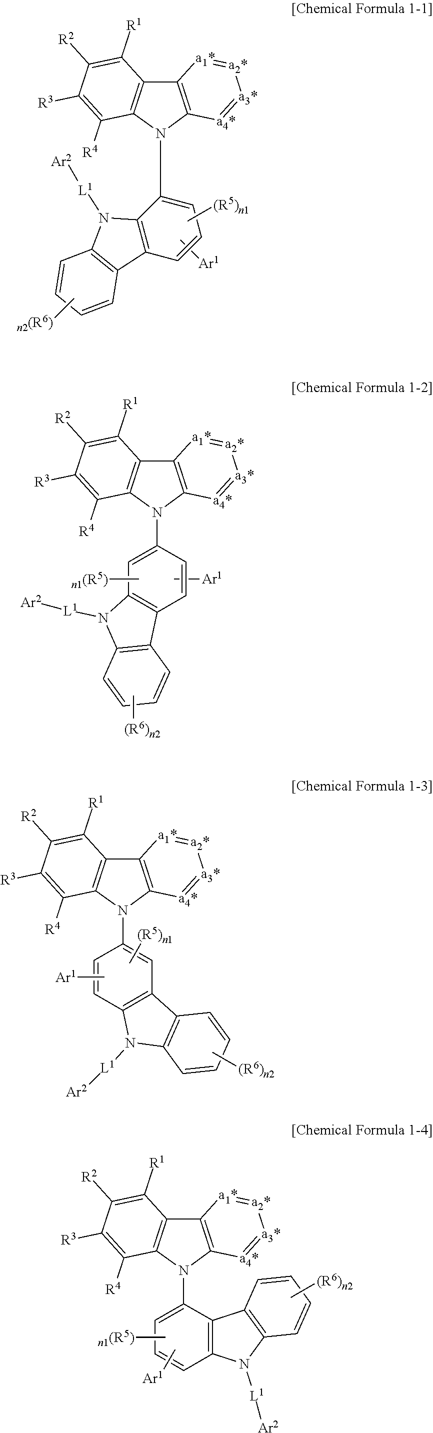

In Chemical Formula 1-1 to Chemical Formula 1-4,
the definitions of Ar¹, Ar², R¹ to R⁶, L¹, n1 and n2, and a1* to a4* are the same as described above.

As an example, the combination of Chemical Formula 1 and Chemical Formula 2 may be represented by any one of Chemical Formula 1A to Chemical Formula 1F.

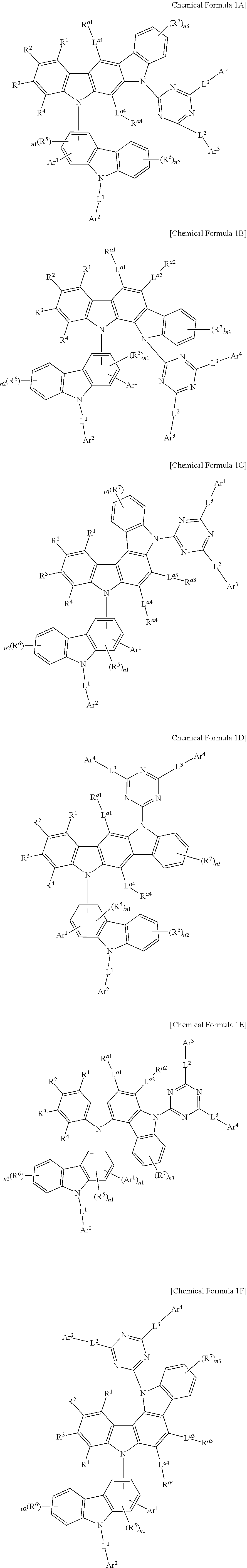

In Chemical Formula 1A to Chemical Formula 1F,
the definitions of Ar¹ to Ar⁴, R¹ to R⁷, L¹ to L³, and n1 to n3 are the same as described above, L^{a1} to L^{a4} are each independently the same as the definitions of L^{a}, and R^{a1} to R^{a4} are each independently the same as the definition of R^{a}.

Specifically, Chemical Formula 1A may be represented by any one of Chemical Formula 1-1A, Chemical Formula 1-2A, Chemical Formula 1-3A, and Chemical Formula 1-4A.

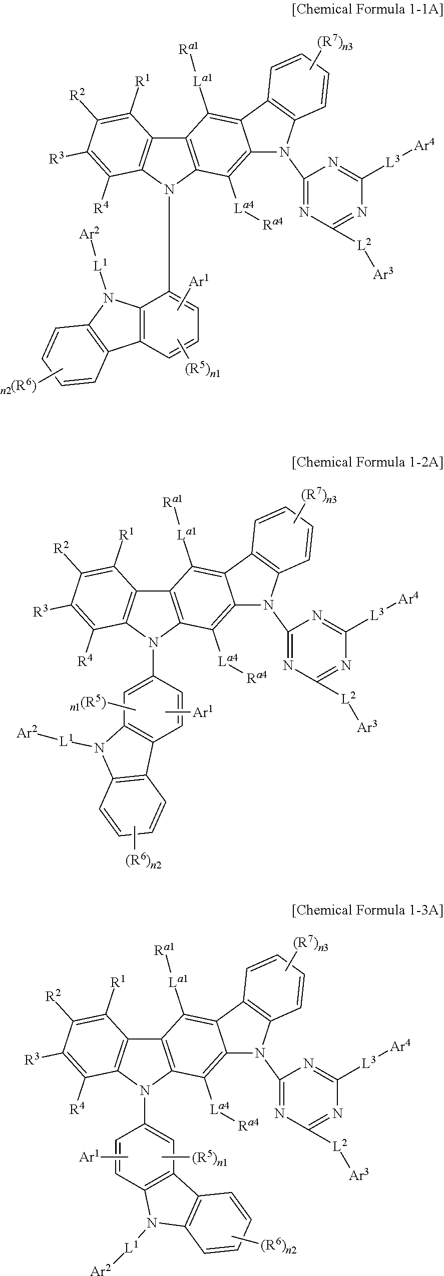

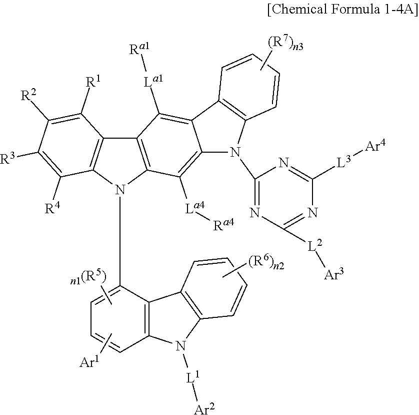

In Chemical Formula 1-1A, Chemical Formula 1-2A, Chemical Formula 1-3A, and Chemical Formula 1-4A,
the definitions of Ar¹ to Ar⁴, R¹ to R⁷, R^{a1}, R^{a4}, L¹ to L³, L^{a1}, L^{a4}, L¹ to L³ and n1 to n3 are the same as described above.

Specifically, Chemical Formula 1B may be represented by any one of Chemical Formula 1-1B, Chemical Formula 1-2B, Chemical Formula 1-3B, and Chemical Formula 1-4B.

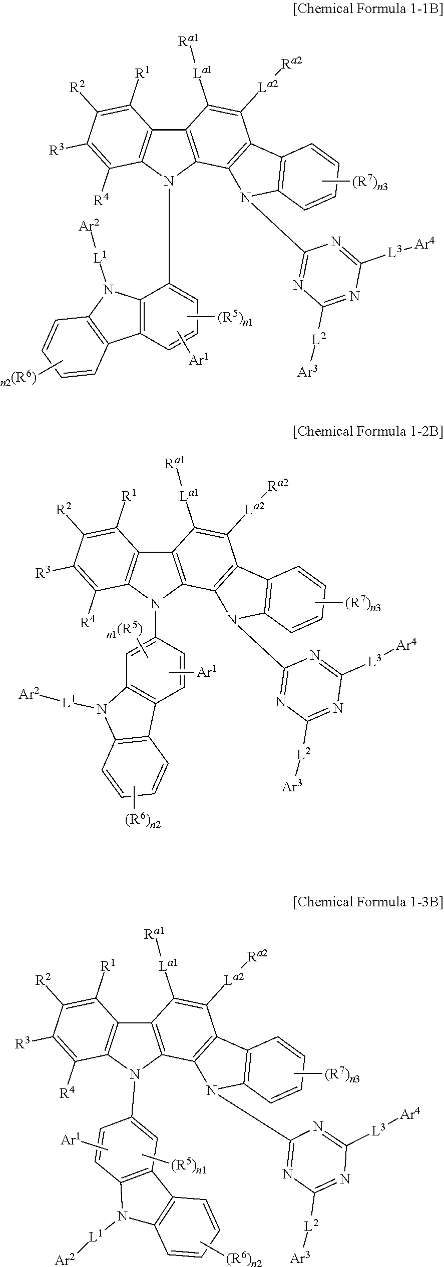

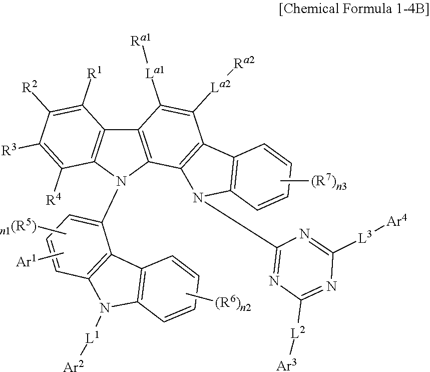

In Chemical Formula 1-1B, Chemical Formula 1-2B, Chemical Formula 1-3B, and Chemical Formula 1-4B,
the definitions of Ar¹ to Ar⁴, R¹ to R⁷, R^{a1}, R^{a2}, L¹ to L³, L^{a1}, L^{a2}, L¹ to L³, and n1 to n3 are the same as described above.

Specifically, Chemical Formula 1C may be represented by any one of Chemical Formula 1-1C, Chemical Formula 1-2C, Chemical Formula 1-3C, and Chemical Formula 1-4C.

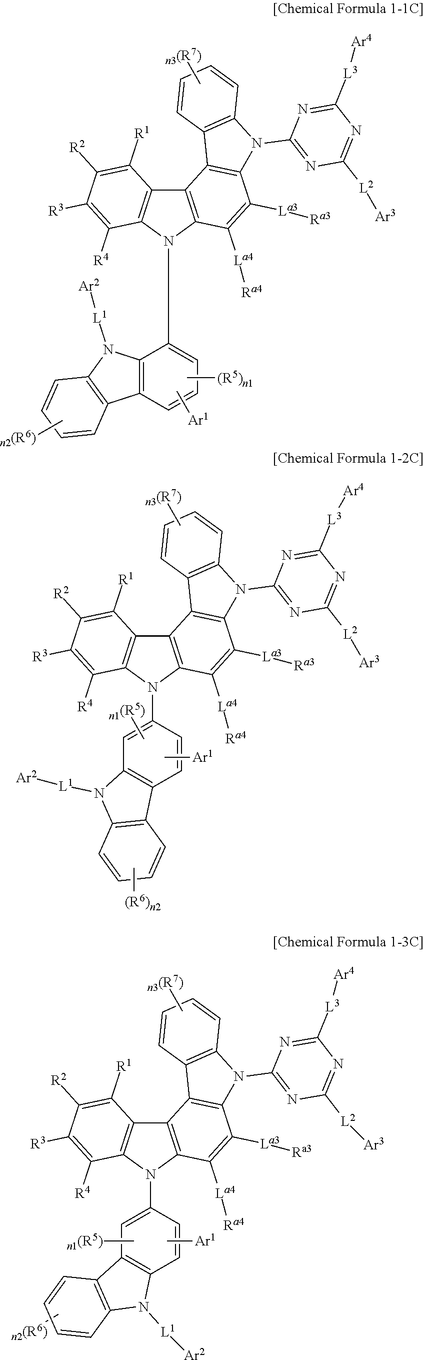

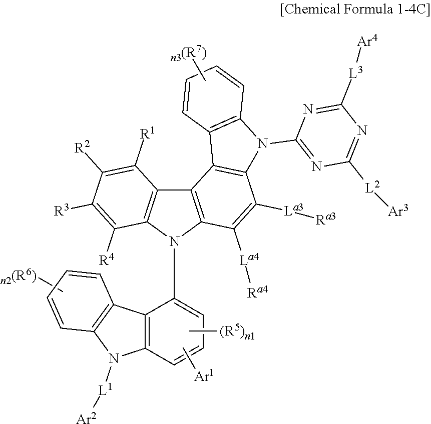

In Chemical Formula 1-1C, Chemical Formula 1-2C, Chemical Formula 1-3C, and Chemical Formula 1-4C,
the definitions of Ar¹ to Ar⁴, R¹ to R, R^{a3}, R^{a4}, L¹ to L³, L^{a3}, L^{a4}, L¹ to L³, and n1 to n3 are the same as described above.

Chemical Formula 1D may be represented by any one of Chemical Formula 1-1D, Chemical Formula 1-2D, Chemical Formula 1-3D, and Chemical Formula 1-4D.

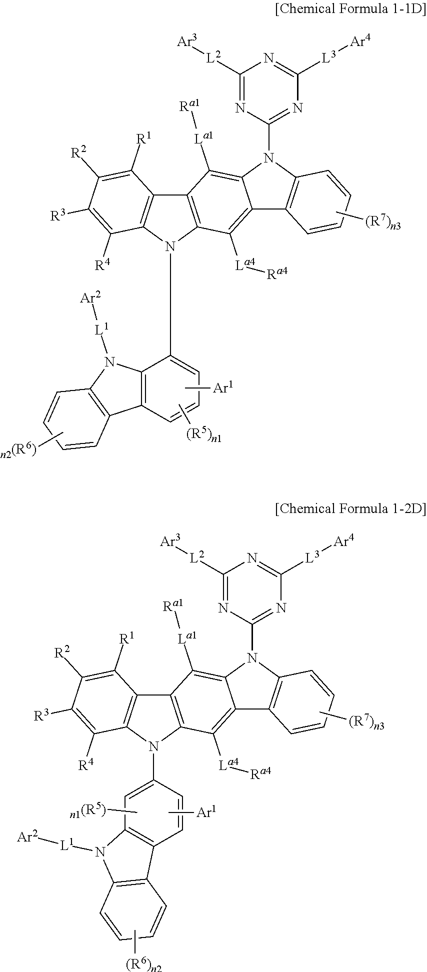

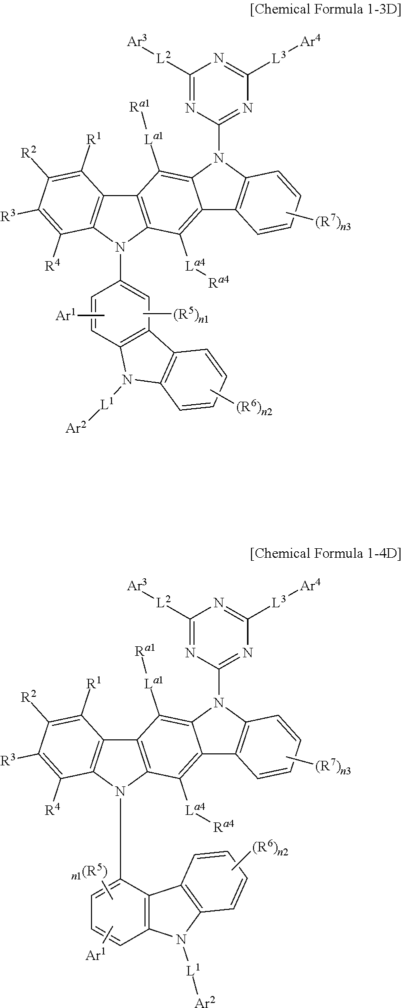

In Chemical Formula 1-1D, Chemical Formula 1-2D, Chemical Formula 1-3D, and Chemical Formula 1-4D,
the definitions of Ar¹ to Ar⁴, R¹ to R⁷, R^{a1}, R^{a4}, L¹ to L³, L^{a1}, L^{a4}, L¹ to L³, and n1 to n3 are the same as described above.

Chemical Formula 1E may be represented by any one of Chemical Formula 1-1E, Chemical Formula 1-2E, Chemical Formula 1-3E, and Chemical Formula 1-4E.

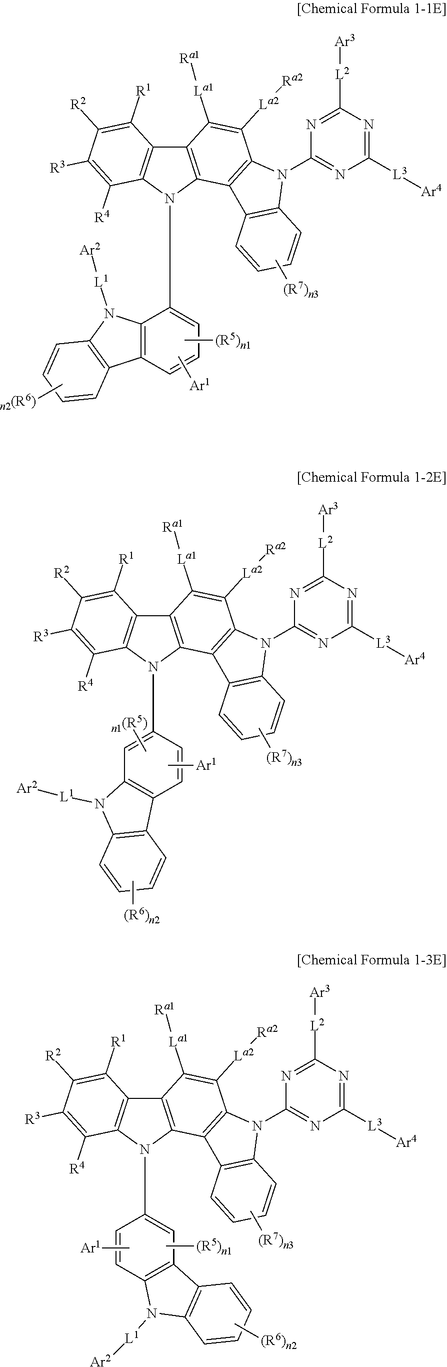

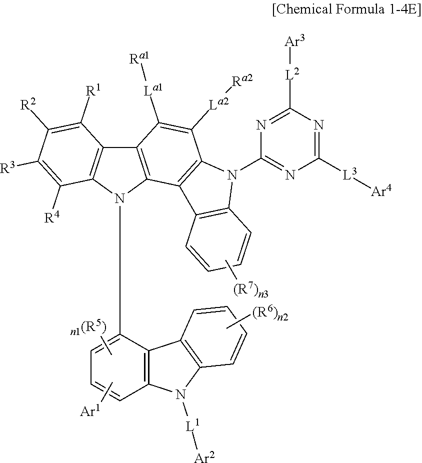

In Chemical Formula 1-1E, Chemical Formula 1-2E, Chemical Formula 1-3E, and Chemical Formula 1-4E,
the definitions of Ar¹ to Ar⁴, R¹ to R⁷, R^{a1}, R^{a2}, L¹ to L³, L^{a1}, L^{a2}, L¹ to L³, and n1 to n3 are the same as described above.

Chemical Formula 1F may be represented by any one of Chemical Formula 1-1F, Chemical Formula 1-2F, Chemical Formula 1-3F, and Chemical Formula 1-4F.

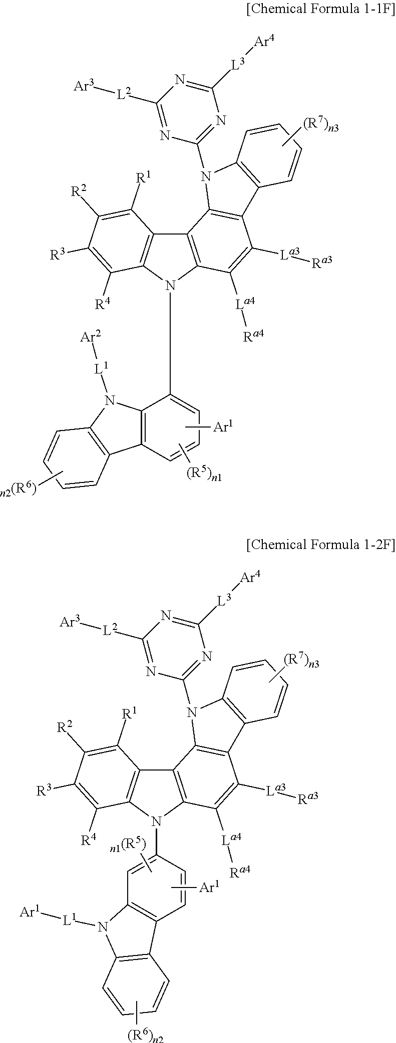

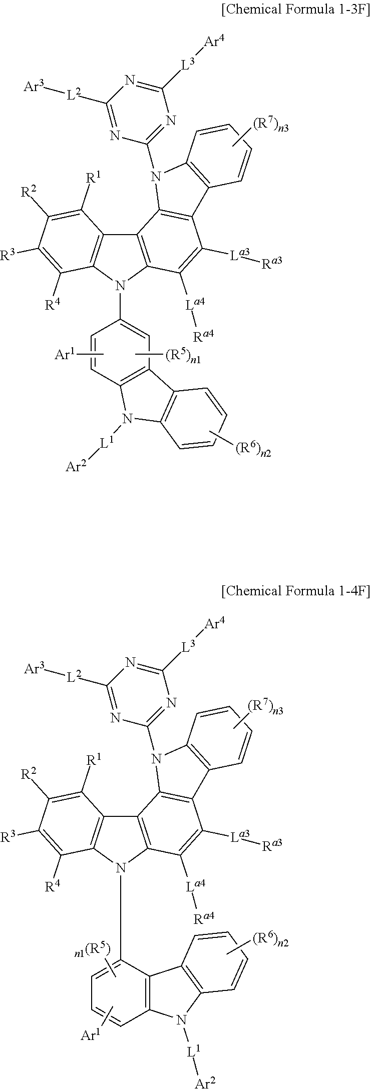

In Chemical Formula 1-1F, Chemical Formula 1-2F, Chemical Formula 1-3F, and Chemical Formula 1-4F,
the definitions of Ar¹ to Ar⁴, R¹ to R⁷, R^{a3}, R^{a4}, L¹ to L³, L^{a3}, L^{a4}, L¹ to L³, and n1 to n3 are the same as described above.

As a specific example, a compound for an organic optoelectronic device represented by a combination of Chemical Formula 1 and Chemical Formula 2 may be represented by any one of Chemical Formula 1-2B, Chemical Formula 1-3B, Chemical Formula 1-2E, and Chemical Formula 1-3E.

In an embodiment, Ar¹ may be a substituted or unsubstituted C6 to C20 aryl group, a substituted or unsubstituted dibenzofuranyl group, a substituted or unsubstituted dibenzothiophenyl group, or a substituted or unsubstituted carbazolyl group.

In a specific embodiment, Ar¹ may be a substituted or unsubstituted C6 to C12 aryl group, a substituted or unsubstituted dibenzofuranyl group, a substituted or unsubstituted dibenzothiophenyl group, or a substituted or unsubstituted carbazolyl group.

For example, Ar¹ may be a substituted or unsubstituted phenyl group or a substituted or unsubstituted biphenyl group.

In an embodiment, Ar² may be a substituted or unsubstituted C6 to C20 aryl group.

In a specific embodiment, Ar² may be a substituted or unsubstituted C6 to C12 aryl group.

For example, Ar² may be a substituted or unsubstituted phenyl group.

In an embodiment, Ar³ and Ar⁴ may each independently be a substituted or unsubstituted C6 to C20 aryl group, a substituted or unsubstituted dibenzofuranyl group, a substituted or unsubstituted dibenzothiophenyl group, or a substituted or unsubstituted dibenzosilolyl group.

In a specific embodiment, Ar³ and Ar⁴ may each independently be a substituted or unsubstituted phenyl group, a substituted or unsubstituted biphenyl group, a substituted or unsubstituted naphthyl group, a substituted or unsubstituted triphenylene group, a substituted or unsubstituted fluorenyl group, a substituted or unsubstituted dibenzofuranyl group, a substituted or unsubstituted dibenzothiophenyl group, or a substituted or unsubstituted dibenzosilolyl group.

In an embodiment, L² and L³ may each independently be a single bond or a substituted or unsubstituted C6 to C12 arylene group.

For example, *-L²-Ar³ and L³-Ar⁴ may each independently be selected from the substituents listed in Group I.

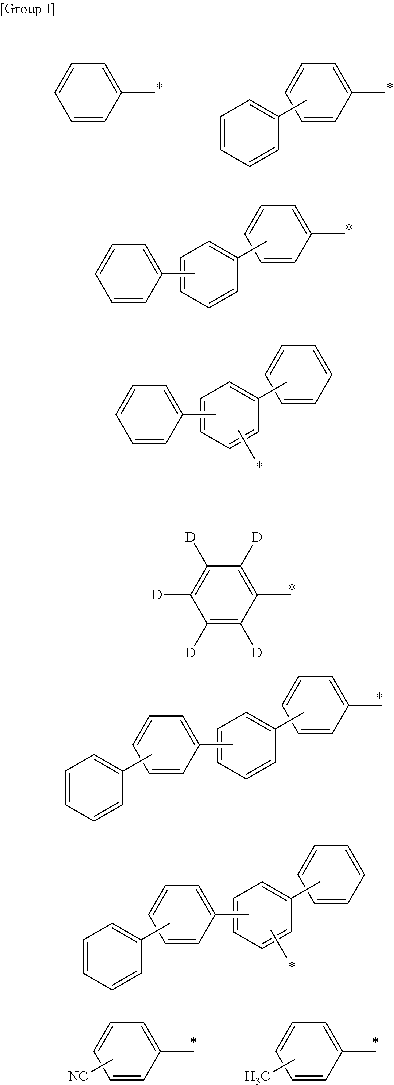

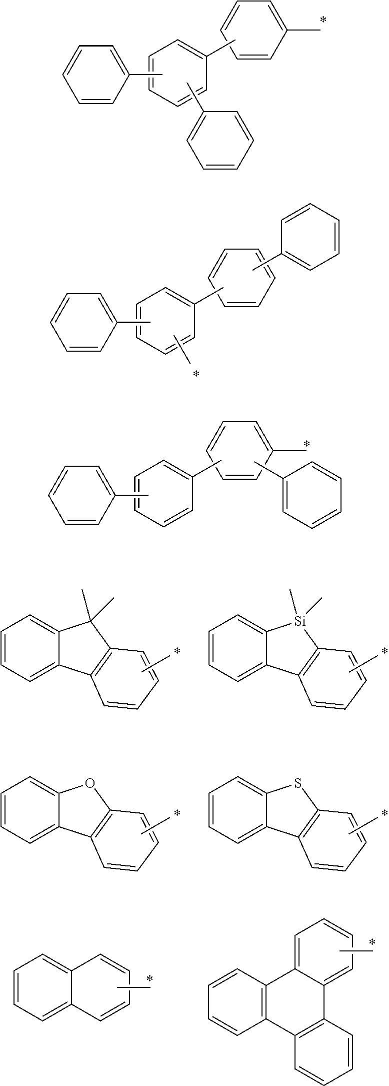

In Group I, each substituent may be unsubstituted or substituted witn h an additional substituent, and * is a linking point.

The additional substituent may be deuterium, a substituted or unsubstituted C1 to C5 alkyl group, or a substituted or unsubstituted C6 to C18 aryl group.
in an embodiment, R¹ to R⁷ and R^{a1} to R^{a4} may each independently be hydrogen, deuterium, a cyano group, a substituted or unsubstituted C1 to C10 alkyl group, a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, or a substituted or unsubstituted triphenylene group.

For example, R¹ to R⁷ and R^{a1} to R^{a4} may each independently be hydrogen, deuterium, a cyano group, a substituted or unsubstituted C1 to C5 alkyl group, or a substituted or unsubstituted phenyl group.

In the most specific embodiment, the compound for an organic optoelectronic device represented by a combination of Chemical Formula 1 and Chemical Formula 2 may be one selected from the compounds listed in Group 1 below, but is not limited thereto.

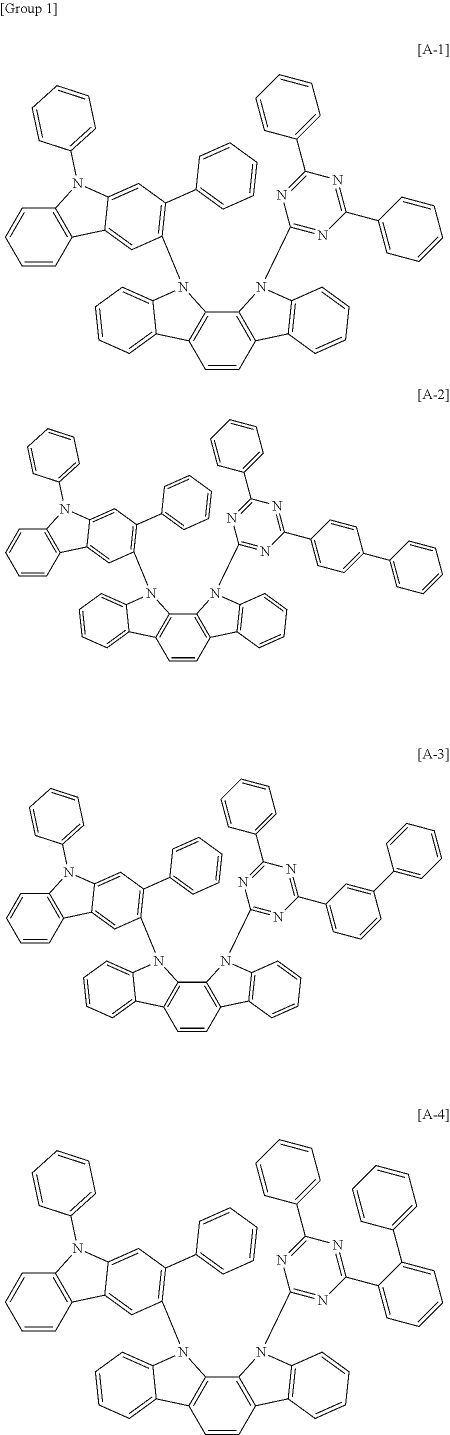 [SMILES: c1ccc(-c2nc(-c3ccccc3)nc(-n3c4ccccc4c4ccc5c6ccccc6n(-c6cc7c8ccccc8n(-c8ccccc8)c7cc6-c6ccccc6)c5c43)n2)cc1 ; c1ccc(-c2ccc(-c3nc(-c4ccccc4)nc(-n4c5ccccc5c5ccc6c7ccccc7n(-c7cc8c9ccccc9n(-c9ccccc9)c8cc7-c7ccccc7)c6c54)n3)cc2)cc1 ; c1ccc(-c2cccc(-c3nc(-c4ccccc4)nc(-n4c5ccccc5c5ccc6c7ccccc7n(-c7cc8c9ccccc9n(-c9ccccc9)c8cc7-c7ccccc7)c6c54)n3)c2)cc1 ; c1ccc(-c2nc(-c3ccccc3-c3ccccc3)nc(-n3c4ccccc4c4ccc5c6ccccc6n(-c6cc7c8ccccc8n(-c8ccccc8)c7cc6-c6ccccc6)c5c43)n2)cc1]

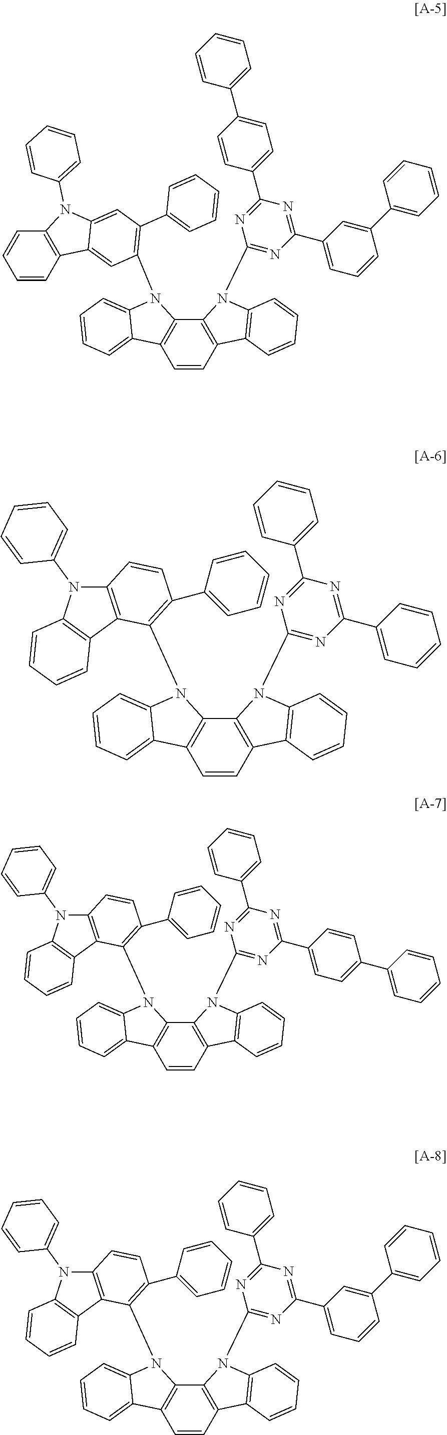 [SMILES: c1ccc(-c2ccc(-c3nc(-c4cccc(-c5ccccc5)c4)nc(-n4c5ccccc5c5ccc6c7ccccc7n(-c7cc8c9ccccc9n(-c9ccccc9)c8cc7-c7ccccc7)c6c54)n3)cc2)cc1 ; c1ccc(-c2nc(-c3ccccc3)nc(-n3c4ccccc4c4ccc5c6ccccc6n(-c6c(-c7ccccc7)ccc7c6c6ccccc6n7-c6ccccc6)c5c43)n2)cc1 ; c1ccc(-c2ccc(-c3nc(-c4ccccc4)nc(-n4c5ccccc5c5ccc6c7ccccc7n(-c7c(-c8ccccc8)ccc8c7c7ccccc7n8-c7ccccc7)c6c54)n3)cc2)cc1 ; c1ccc(-c2cccc(-c3nc(-c4ccccc4)nc(-n4c5ccccc5c5ccc6c7ccccc7n(-c7c(-c8ccccc8)ccc8c7c7ccccc7n8-c7ccccc7)c6c54)n3)c2)cc1]

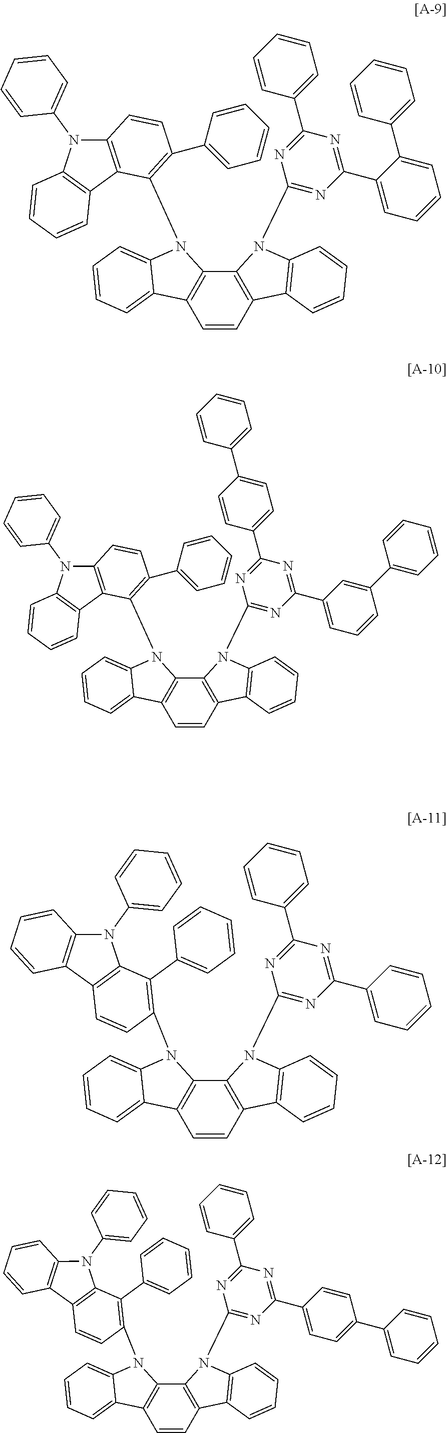 [SMILES: c1ccc(-c2nc(-c3ccccc3-c3ccccc3)nc(-n3c4ccccc4c4ccc5c6ccccc6n(-c6c(-c7ccccc7)ccc7c6c6ccccc6n7-c6ccccc6)c5c43)n2)cc1 ; c1ccc(-c2ccc(-c3nc(-c4cccc(-c5ccccc5)c4)nc(-n4c5ccccc5c5ccc6c7ccccc7n(-c7c(-c8ccccc8)ccc8c7c7ccccc7n8-c7ccccc7)c6c54)n3)cc2)cc1 ; c1ccc(-c2nc(-c3ccccc3)nc(-n3c4ccccc4c4ccc5c6ccccc6n(-c6ccc7c8ccccc8n(-c8ccccc8)c7c6-c6ccccc6)c5c43)n2)cc1 ; c1ccc(-c2ccc(-c3nc(-c4ccccc4)nc(-n4c5ccccc5c5ccc6c7ccccc7n(-c7ccc8c9ccccc9n(-c9ccccc9)c8c7-c7ccccc7)c6c54)n3)cc2)cc1]

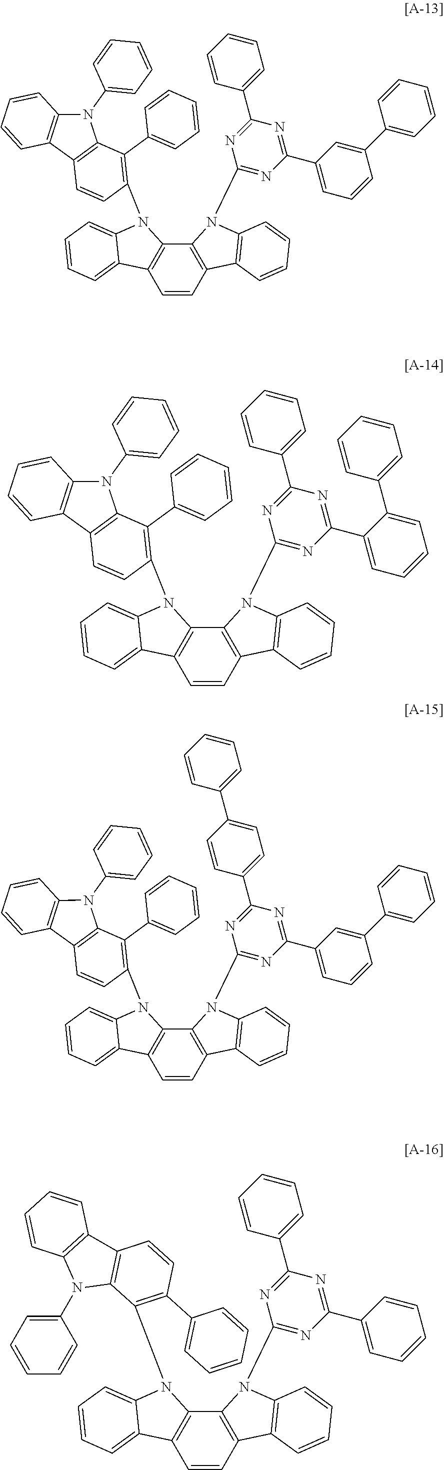 [SMILES: c1ccc(-c2cccc(-c3nc(-c4ccccc4)nc(-n4c5ccccc5c5ccc6c7ccccc7n(-c7ccc8c9ccccc9n(-c9ccccc9)c8c7-c7ccccc7)c6c54)n3)c2)cc1 ; c1ccc(-c2nc(-c3ccccc3-c3ccccc3)nc(-n3c4ccccc4c4ccc5c6ccccc6n(-c6ccc7c8ccccc8n(-c8ccccc8)c7c6-c6ccccc6)c5c43)n2)cc1 ; c1ccc(-c2ccc(-c3nc(-c4cccc(-c5ccccc5)c4)nc(-n4c5ccccc5c5ccc6c7ccccc7n(-c7ccc8c9ccccc9n(-c9ccccc9)c8c7-c7ccccc7)c6c54)n3)cc2)cc1 ; c1ccc(-c2nc(-c3ccccc3)nc(-n3c4ccccc4c4ccc5c6ccccc6n(-c6c(-c7ccccc7)ccc7c8ccccc8n(-c8ccccc8)c67)c5c43)n2)cc1]

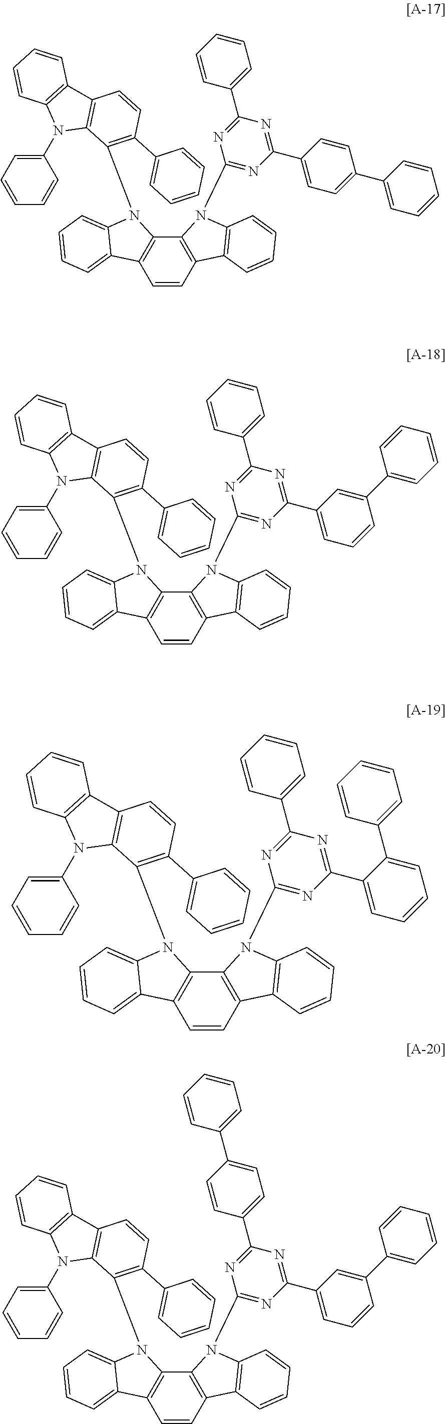 [SMILES: c1ccc(-c2ccc(-c3nc(-c4ccccc4)nc(-n4c5ccccc5c5ccc6c7ccccc7n(-c7c(-c8ccccc8)ccc8c9ccccc9n(-c9ccccc9)c78)c6c54)n3)cc2)cc1 ; c1ccc(-c2cccc(-c3nc(-c4ccccc4)nc(-n4c5ccccc5c5ccc6c7ccccc7n(-c7c(-c8ccccc8)ccc8c9ccccc9n(-c9ccccc9)c78)c6c54)n3)c2)cc1 ; c1ccc(-c2nc(-c3ccccc3-c3ccccc3)nc(-n3c4ccccc4c4ccc5c6ccccc6n(-c6c(-c7ccccc7)ccc7c8ccccc8n(-c8ccccc8)c67)c5c43)n2)cc1 ; c1ccc(-c2ccc(-c3nc(-c4cccc(-c5ccccc5)c4)nc(-n4c5ccccc5c5ccc6c7ccccc7n(-c7c(-c8ccccc8)ccc8c9ccccc9n(-c9ccccc9)c78)c6c54)n3)cc2)cc1]

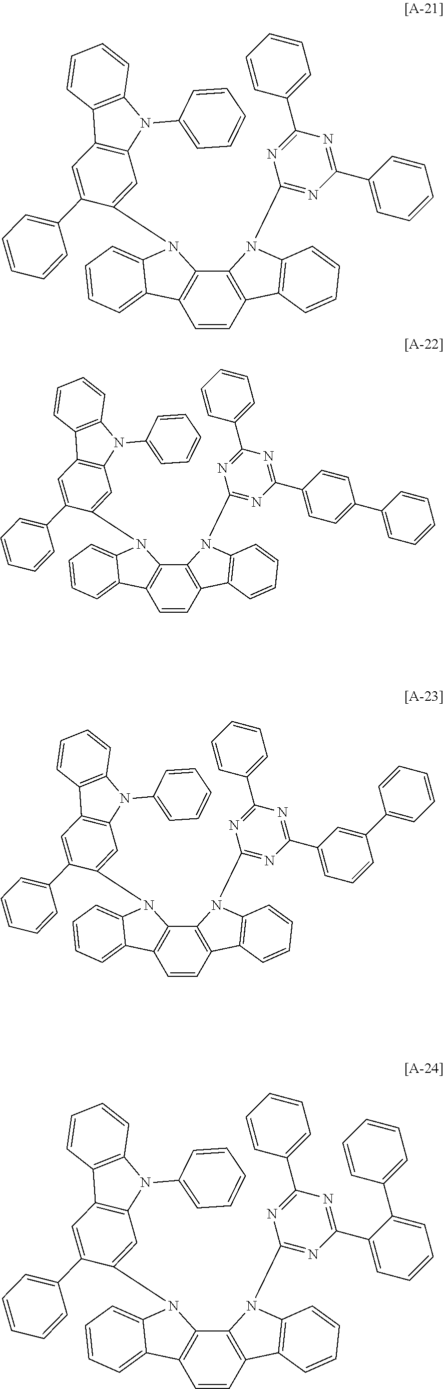

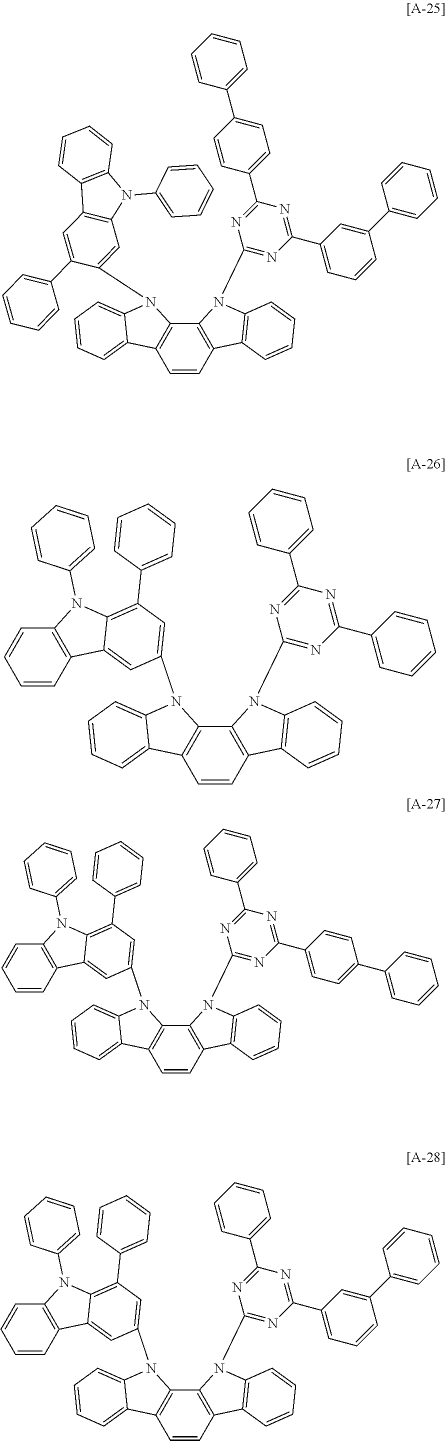 [SMILES: c1ccc(-c2ccc(-c3nc(-c4cccc(-c5ccccc5)c4)nc(-n4c5ccccc5c5ccc6c7ccccc7n(-c7cc8c(cc7-c7ccccc7)c7ccccc7n8-c7ccccc7)c6c54)n3)cc2)cc1 ; c1ccc(-c2nc(-c3ccccc3)nc(-n3c4ccccc4c4ccc5c6ccccc6n(-c6cc(-c7ccccc7)c7c(c6)c6ccccc6n7-c6ccccc6)c5c43)n2)cc1 ; c1ccc(-c2ccc(-c3nc(-c4ccccc4)nc(-n4c5ccccc5c5ccc6c7ccccc7n(-c7cc(-c8ccccc8)c8c(c7)c7ccccc7n8-c7ccccc7)c6c54)n3)cc2)cc1 ; c1ccc(-c2cccc(-c3nc(-c4ccccc4)nc(-n4c5ccccc5c5ccc6c7ccccc7n(-c7cc(-c8ccccc8)c8c(c7)c7ccccc7n8-c7ccccc7)c6c54)n3)c2)cc1]

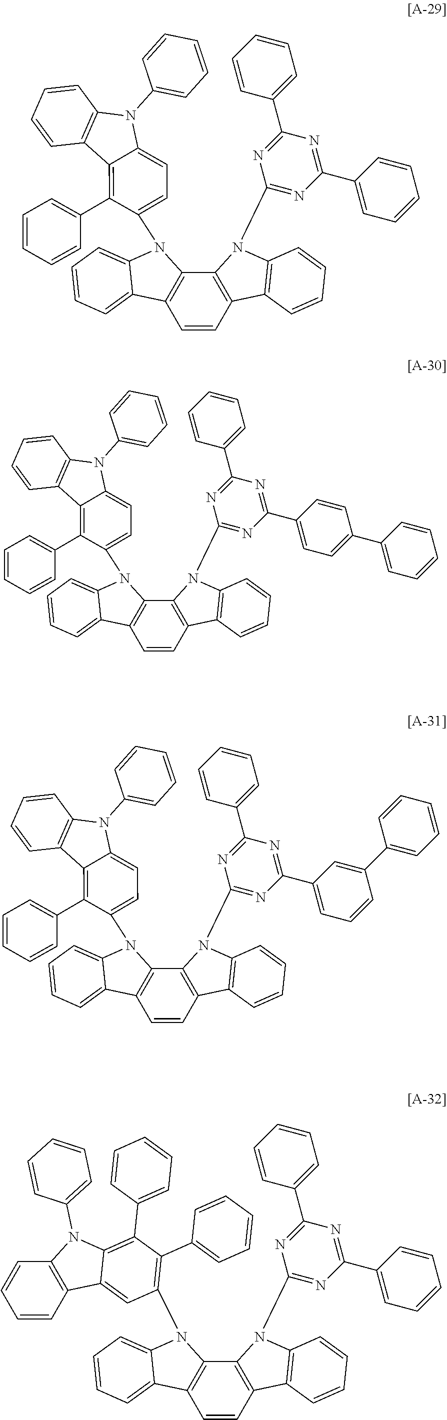 [SMILES: c1ccc(-c2nc(-c3ccccc3)nc(-n3c4ccccc4c4ccc5c6ccccc6n(-c6ccc7c(c6-c6ccccc6)c6ccccc6n7-c6ccccc6)c5c43)n2)cc1 ; c1ccc(-c2ccc(-c3nc(-c4ccccc4)nc(-n4c5ccccc5c5ccc6c7ccccc7n(-c7ccc8c(c7-c7ccccc7)c7ccccc7n8-c7ccccc7)c6c54)n3)cc2)cc1 ; c1ccc(-c2cccc(-c3nc(-c4ccccc4)nc(-n4c5ccccc5c5ccc6c7ccccc7n(-c7ccc8c(c7-c7ccccc7)c7ccccc7n8-c7ccccc7)c6c54)n3)c2)cc1 ; c1ccc(-c2nc(-c3ccccc3)nc(-n3c4ccccc4c4ccc5c6ccccc6n(-c6cc7c8ccccc8n(-c8ccccc8)c7c(-c7ccccc7)c6-c6ccccc6)c5c43)n2)cc1]

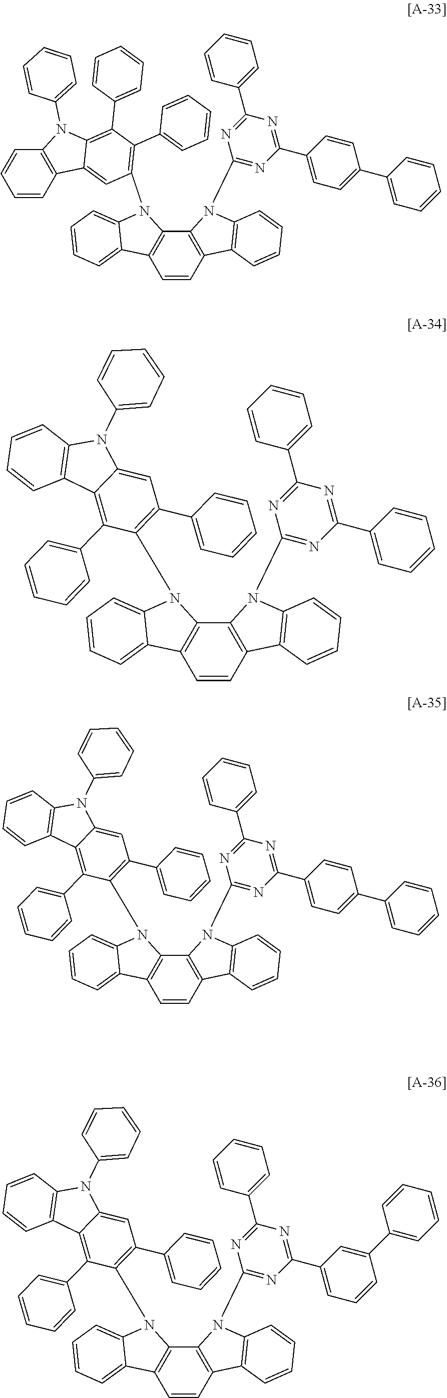

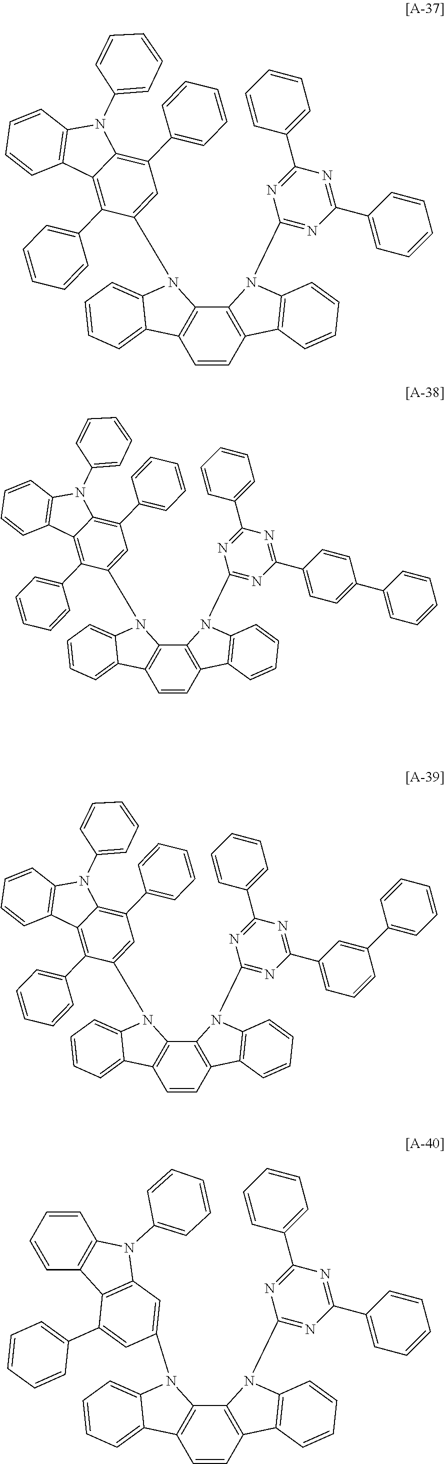 [SMILES: c1ccc(-c2nc(-c3ccccc3)nc(-n3c4ccccc4c4ccc5c6ccccc6n(-c6cc(-c7ccccc7)c7c(c6-c6ccccc6)c6ccccc6n7-c6ccccc6)c5c43)n2)cc1 ; c1ccc(-c2ccc(-c3nc(-c4ccccc4)nc(-n4c5ccccc5c5ccc6c7ccccc7n(-c7cc(-c8ccccc8)c8c(c7-c7ccccc7)c7ccccc7n8-c7ccccc7)c6c54)n3)cc2)cc1 ; c1ccc(-c2cccc(-c3nc(-c4ccccc4)nc(-n4c5ccccc5c5ccc6c7ccccc7n(-c7cc(-c8ccccc8)c8c(c7-c7ccccc7)c7ccccc7n8-c7ccccc7)c6c54)n3)c2)cc1 ; c1ccc(-c2nc(-c3ccccc3)nc(-n3c4ccccc4c4ccc5c6ccccc6n(-c6cc(-c7ccccc7)c7c8ccccc8n(-c8ccccc8)c7c6)c5c43)n2)cc1]

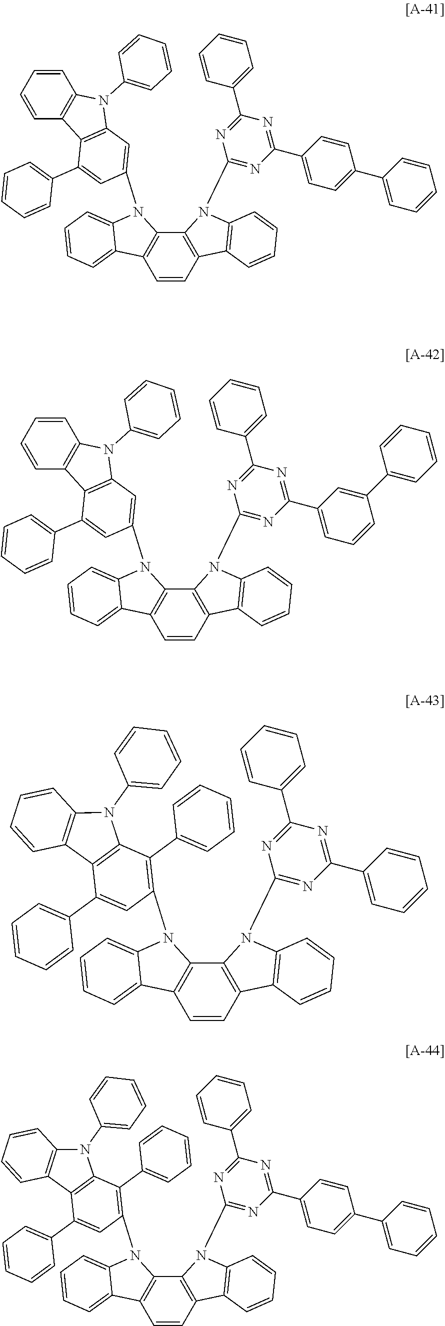 [SMILES: c1ccc(-c2ccc(-c3nc(-c4ccccc4)nc(-n4c5ccccc5c5ccc6c7ccccc7n(-c7cc(-c8ccccc8)c8c9ccccc9n(-c9ccccc9)c8c7)c6c54)n3)cc2)cc1 ; c1ccc(-c2cccc(-c3nc(-c4ccccc4)nc(-n4c5ccccc5c5ccc6c7ccccc7n(-c7cc(-c8ccccc8)c8c9ccccc9n(-c9ccccc9)c8c7)c6c54)n3)c2)cc1 ; c1ccc(-c2nc(-c3ccccc3)nc(-n3c4ccccc4c4ccc5c6ccccc6n(-c6cc(-c7ccccc7)c7c8ccccc8n(-c8ccccc8)c7c6-c6ccccc6)c5c43)n2)cc1 ; c1ccc(-c2ccc(-c3nc(-c4ccccc4)nc(-n4c5ccccc5c5ccc6c7ccccc7n(-c7cc(-c8ccccc8)c8c9ccccc9n(-c9ccccc9)c8c7-c7ccccc7)c6c54)n3)cc2)cc1]

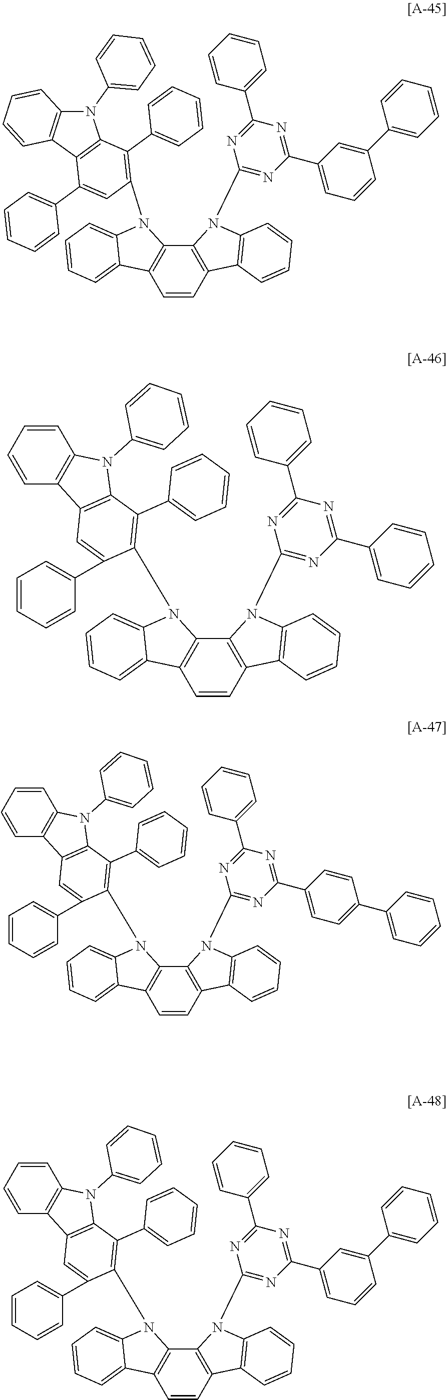

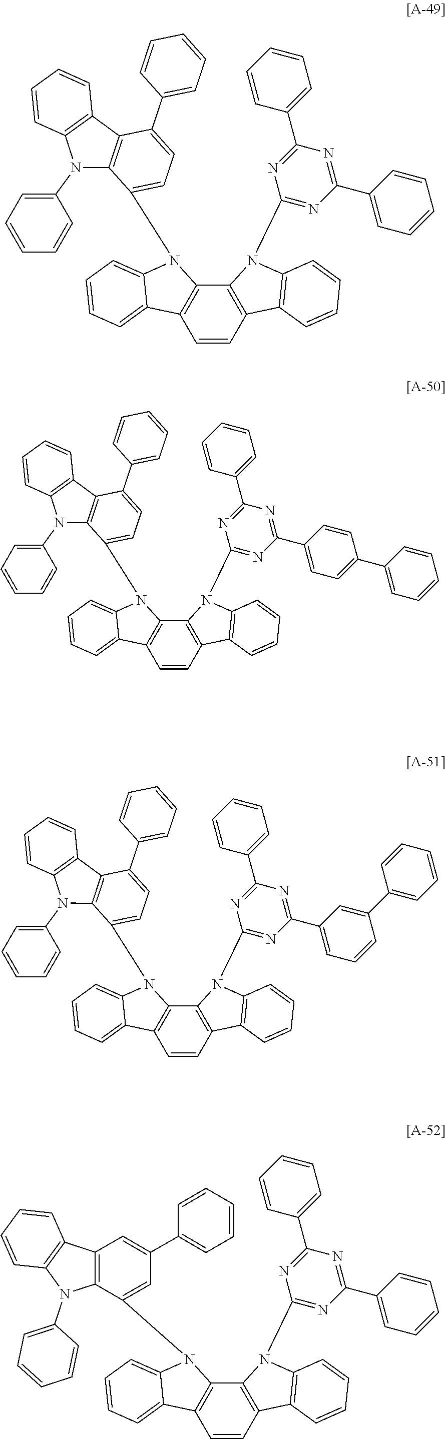 [SMILES: c1ccc(-c2nc(-c3ccccc3)nc(-n3c4ccccc4c4ccc5c6ccccc6n(-c6ccc(-c7ccccc7)c7c8ccccc8n(-c8ccccc8)c67)c5c43)n2)cc1 ; c1ccc(-c2ccc(-c3nc(-c4ccccc4)nc(-n4c5ccccc5c5ccc6c7ccccc7n(-c7ccc(-c8ccccc8)c8c9ccccc9n(-c9ccccc9)c78)c6c54)n3)cc2)cc1 ; c1ccc(-c2cccc(-c3nc(-c4ccccc4)nc(-n4c5ccccc5c5ccc6c7ccccc7n(-c7ccc(-c8ccccc8)c8c9ccccc9n(-c9ccccc9)c78)c6c54)n3)c2)cc1 ; c1ccc(-c2cc(-n3c4ccccc4c4ccc5c6ccccc6n(-c6nc(-c7ccccc7)nc(-c7ccccc7)n6)c5c43)c3c(c2)c2ccccc2n3-c2ccccc2)cc1]

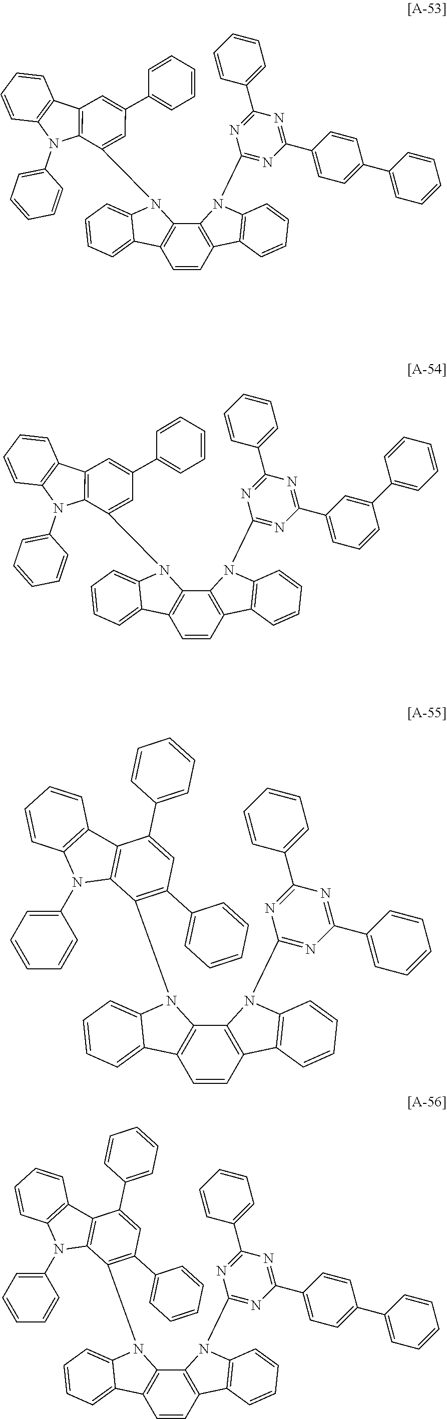 [SMILES: c1ccc(-c2ccc(-c3nc(-c4ccccc4)nc(-n4c5ccccc5c5ccc6c7ccccc7n(-c7cc(-c8ccccc8)cc8c9ccccc9n(-c9ccccc9)c78)c6c54)n3)cc2)cc1 ; c1ccc(-c2cccc(-c3nc(-c4ccccc4)nc(-n4c5ccccc5c5ccc6c7ccccc7n(-c7cc(-c8ccccc8)cc8c9ccccc9n(-c9ccccc9)c78)c6c54)n3)c2)cc1 ; c1ccc(-c2nc(-c3ccccc3)nc(-n3c4ccccc4c4ccc5c6ccccc6n(-c6c(-c7ccccc7)cc(-c7ccccc7)c7c8ccccc8n(-c8ccccc8)c67)c5c43)n2)cc1 ; c1ccc(-c2ccc(-c3nc(-c4ccccc4)nc(-n4c5ccccc5c5ccc6c7ccccc7n(-c7c(-c8ccccc8)cc(-c8ccccc8)c8c9ccccc9n(-c9ccccc9)c78)c6c54)n3)cc2)cc1]

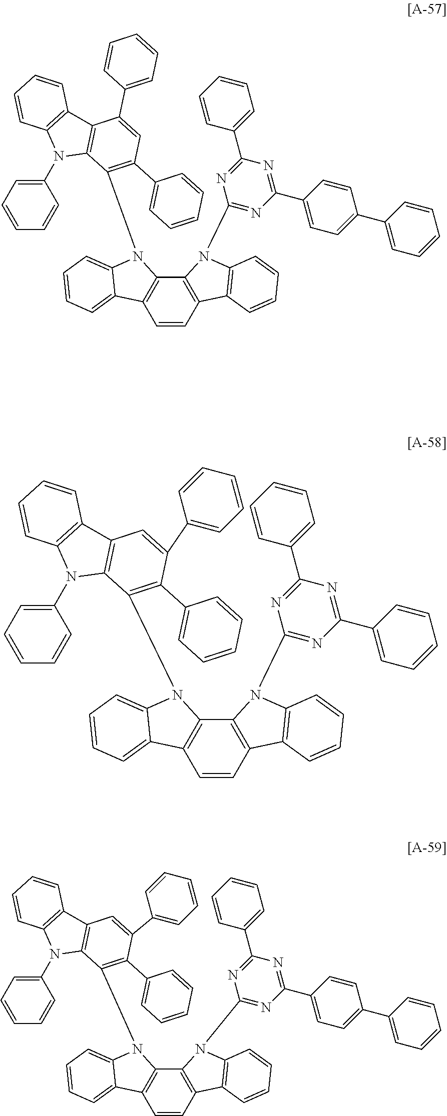 [SMILES: c1ccc(-c2ccc(-c3nc(-c4ccccc4)nc(-n4c5ccccc5c5ccc6c7ccccc7n(-c7c(-c8ccccc8)cc(-c8ccccc8)c8c9ccccc9n(-c9ccccc9)c78)c6c54)n3)cc2)cc1 ; c1ccc(-c2nc(-c3ccccc3)nc(-n3c4ccccc4c4ccc5c6ccccc6n(-c6c(-c7ccccc7)c(-c7ccccc7)cc7c8ccccc8n(-c8ccccc8)c67)c5c43)n2)cc1 ; c1ccc(-c2ccc(-c3nc(-c4ccccc4)nc(-n4c5ccccc5c5ccc6c7ccccc7n(-c7c(-c8ccccc8)c(-c8ccccc8)cc8c9ccccc9n(-c9ccccc9)c78)c6c54)n3)cc2)cc1]

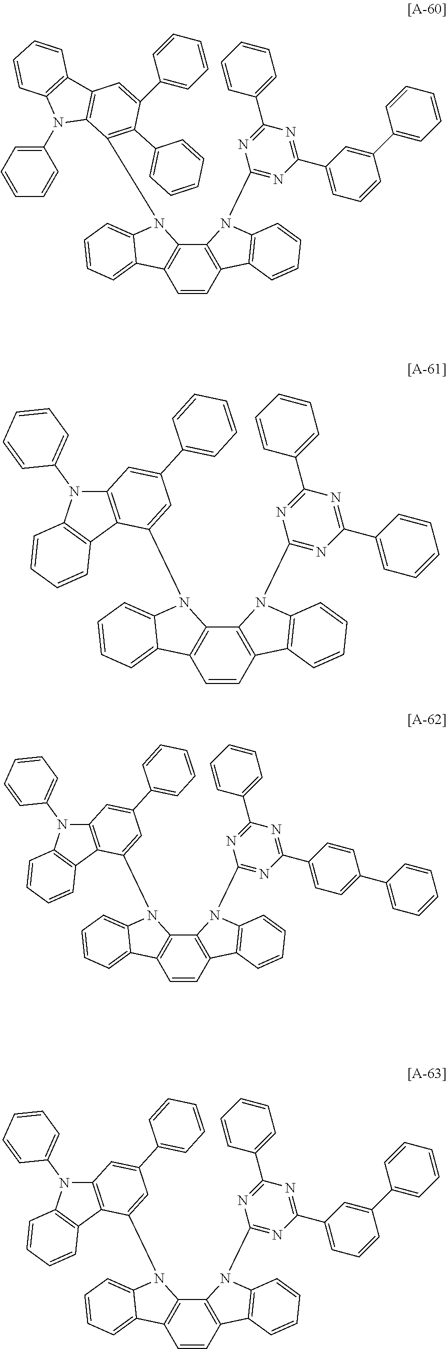 [SMILES: c1ccc(-c2cccc(-c3nc(-c4ccccc4)nc(-n4c5ccccc5c5ccc6c7ccccc7n(-c7c(-c8ccccc8)c(-c8ccccc8)cc8c9ccccc9n(-c9ccccc9)c78)c6c54)n3)c2)cc1 ; c1ccc(-c2cc(-n3c4ccccc4c4ccc5c6ccccc6n(-c6nc(-c7ccccc7)nc(-c7ccccc7)n6)c5c43)c3c4ccccc4n(-c4ccccc4)c3c2)cc1 ; c1ccc(-c2ccc(-c3nc(-c4ccccc4)nc(-n4c5ccccc5c5ccc6c7ccccc7n(-c7cc(-c8ccccc8)cc8c7c7ccccc7n8-c7ccccc7)c6c54)n3)cc2)cc1 ; c1ccc(-c2cccc(-c3nc(-c4ccccc4)nc(-n4c5ccccc5c5ccc6c7ccccc7n(-c7cc(-c8ccccc8)cc8c7c7ccccc7n8-c7ccccc7)c6c54)n3)c2)cc1]

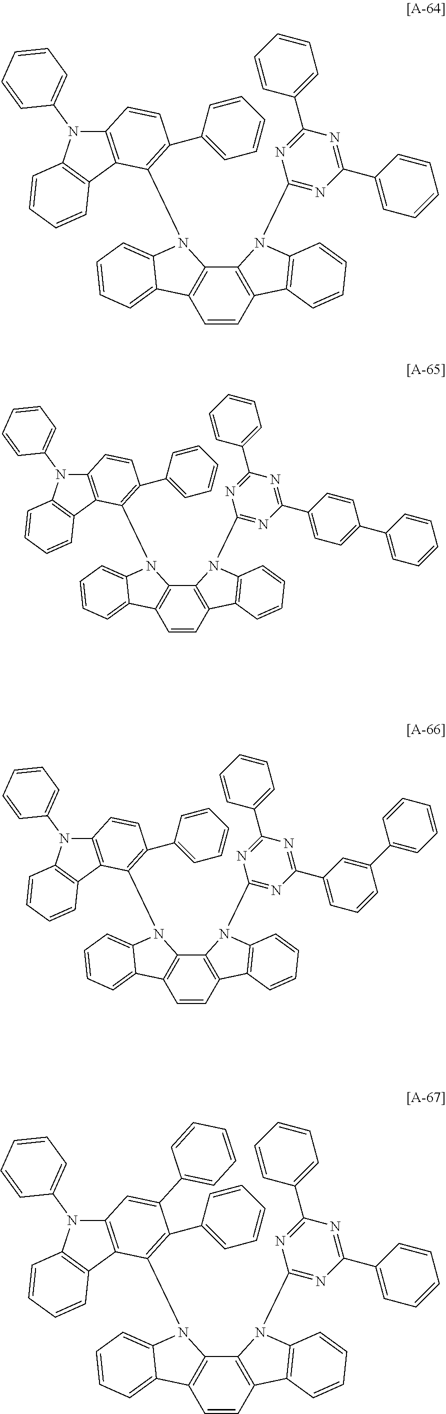 [SMILES: c1ccc(-c2nc(-c3ccccc3)nc(-n3c4ccccc4c4ccc5c6ccccc6n(-c6c(-c7ccccc7)ccc7c6c6ccccc6n7-c6ccccc6)c5c43)n2)cc1 ; c1ccc(-c2ccc(-c3nc(-c4ccccc4)nc(-n4c5ccccc5c5ccc6c7ccccc7n(-c7c(-c8ccccc8)ccc8c7c7ccccc7n8-c7ccccc7)c6c54)n3)cc2)cc1 ; c1ccc(-c2cccc(-c3nc(-c4ccccc4)nc(-n4c5ccccc5c5ccc6c7ccccc7n(-c7c(-c8ccccc8)ccc8c7c7ccccc7n8-c7ccccc7)c6c54)n3)c2)cc1 ; c1ccc(-c2nc(-c3ccccc3)nc(-n3c4ccccc4c4ccc5c6ccccc6n(-c6c(-c7ccccc7)c(-c7ccccc7)cc7c6c6ccccc6n7-c6ccccc6)c5c43)n2)cc1]

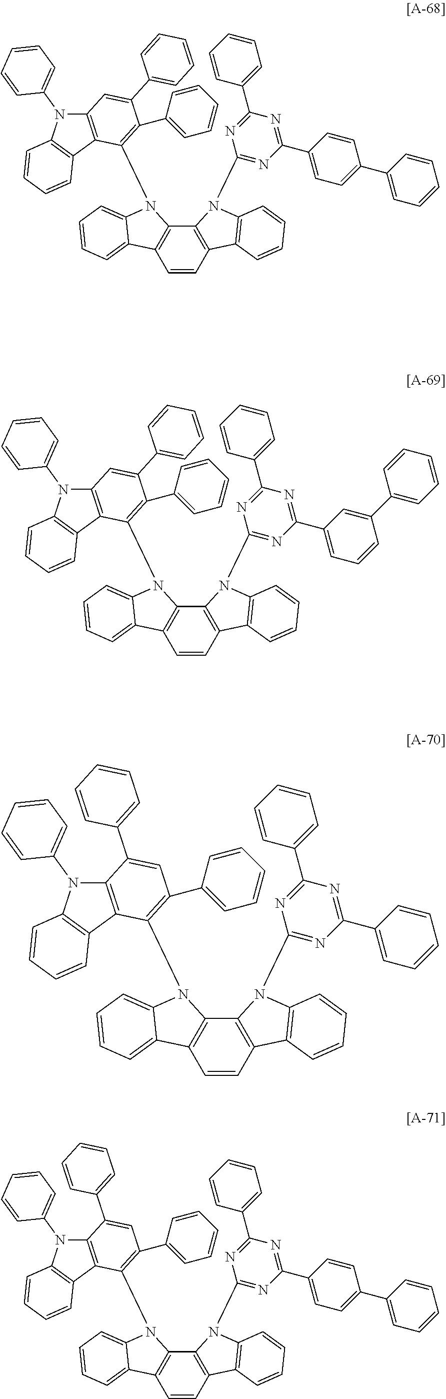

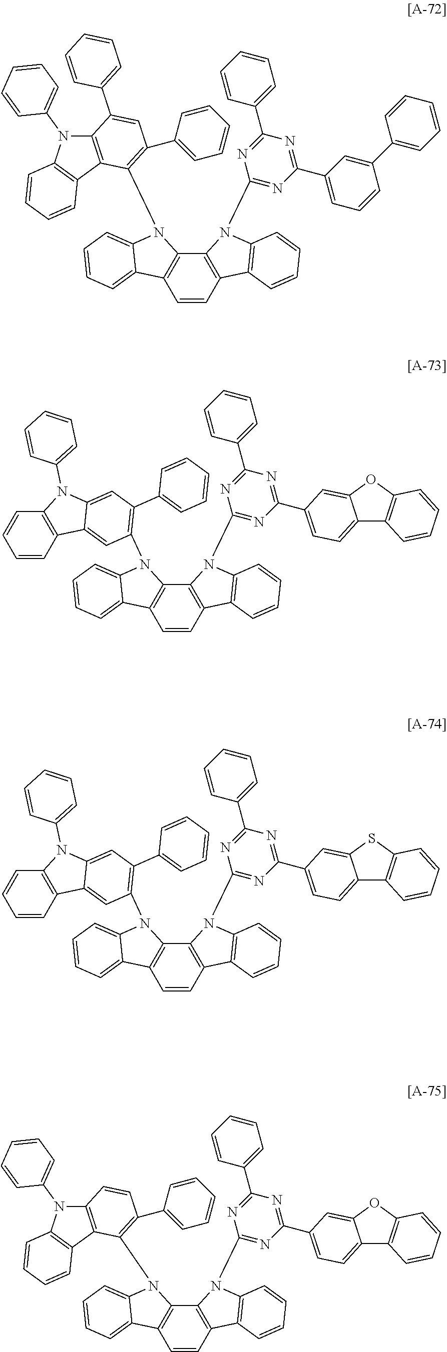 [SMILES: c1ccc(-c2cccc(-c3nc(-c4ccccc4)nc(-n4c5ccccc5c5ccc6c7ccccc7n(-c7c(-c8ccccc8)cc(-c8ccccc8)c8c7c7ccccc7n8-c7ccccc7)c6c54)n3)c2)cc1 ; c1ccc(-c2nc(-c3ccc4c(c3)oc3ccccc34)nc(-n3c4ccccc4c4ccc5c6ccccc6n(-c6cc7c8ccccc8n(-c8ccccc8)c7cc6-c6ccccc6)c5c43)n2)cc1 ; c1ccc(-c2nc(-c3ccc4c(c3)sc3ccccc34)nc(-n3c4ccccc4c4ccc5c6ccccc6n(-c6cc7c8ccccc8n(-c8ccccc8)c7cc6-c6ccccc6)c5c43)n2)cc1 ; c1ccc(-c2nc(-c3ccc4c(c3)oc3ccccc34)nc(-n3c4ccccc4c4ccc5c6ccccc6n(-c6c(-c7ccccc7)ccc7c6c6ccccc6n7-c6ccccc6)c5c43)n2)cc1]

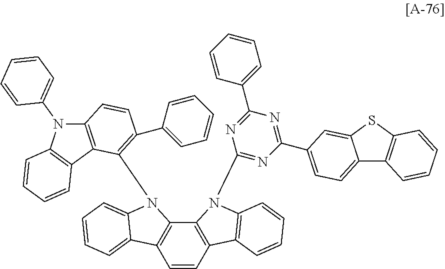 [SMILES: c1ccc(-c2nc(-c3ccc4c(c3)sc3ccccc34)nc(-n3c4ccccc4c4ccc5c6ccccc6n(-c6c(-c7ccccc7)ccc7c6c6ccccc6n7-c6ccccc6)c5c43)n2)cc1]

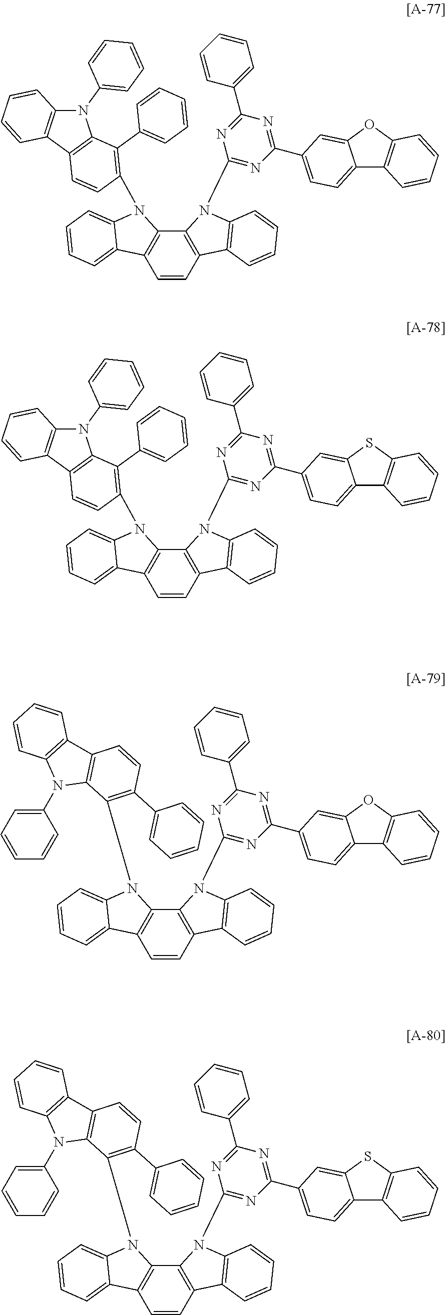 [SMILES: c1ccc(-c2nc(-c3ccc4c(c3)oc3ccccc34)nc(-n3c4ccccc4c4ccc5c6ccccc6n(-c6ccc7c8ccccc8n(-c8ccccc8)c7c6-c6ccccc6)c5c43)n2)cc1 ; c1ccc(-c2nc(-c3ccc4c(c3)sc3ccccc34)nc(-n3c4ccccc4c4ccc5c6ccccc6n(-c6ccc7c8ccccc8n(-c8ccccc8)c7c6-c6ccccc6)c5c43)n2)cc1 ; c1ccc(-c2nc(-c3ccc4c(c3)oc3ccccc34)nc(-n3c4ccccc4c4ccc5c6ccccc6n(-c6c(-c7ccccc7)ccc7c8ccccc8n(-c8ccccc8)c67)c5c43)n2)cc1 ; c1ccc(-c2nc(-c3ccc4c(c3)sc3ccccc34)nc(-n3c4ccccc4c4ccc5c6ccccc6n(-c6c(-c7ccccc7)ccc7c8ccccc8n(-c8ccccc8)c67)c5c43)n2)cc1]

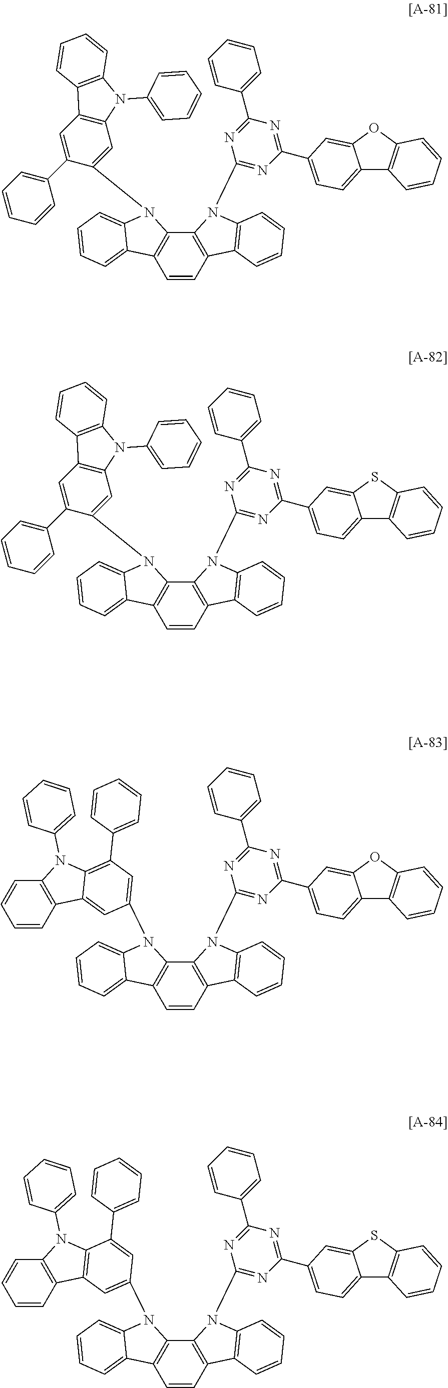 [SMILES: c1ccc(-c2nc(-c3ccc4c(c3)oc3ccccc34)nc(-n3c4ccccc4c4ccc5c6ccccc6n(-c6cc7c(cc6-c6ccccc6)c6ccccc6n7-c6ccccc6)c5c43)n2)cc1 ; c1ccc(-c2nc(-c3ccc4c(c3)sc3ccccc34)nc(-n3c4ccccc4c4ccc5c6ccccc6n(-c6cc7c(cc6-c6ccccc6)c6ccccc6n7-c6ccccc6)c5c43)n2)cc1 ; c1ccc(-c2nc(-c3ccc4c(c3)oc3ccccc34)nc(-n3c4ccccc4c4ccc5c6ccccc6n(-c6cc(-c7ccccc7)c7c(c6)c6ccccc6n7-c6ccccc6)c5c43)n2)cc1 ; c1ccc(-c2nc(-c3ccc4c(c3)sc3ccccc34)nc(-n3c4ccccc4c4ccc5c6ccccc6n(-c6cc(-c7ccccc7)c7c(c6)c6ccccc6n7-c6ccccc6)c5c43)n2)cc1]

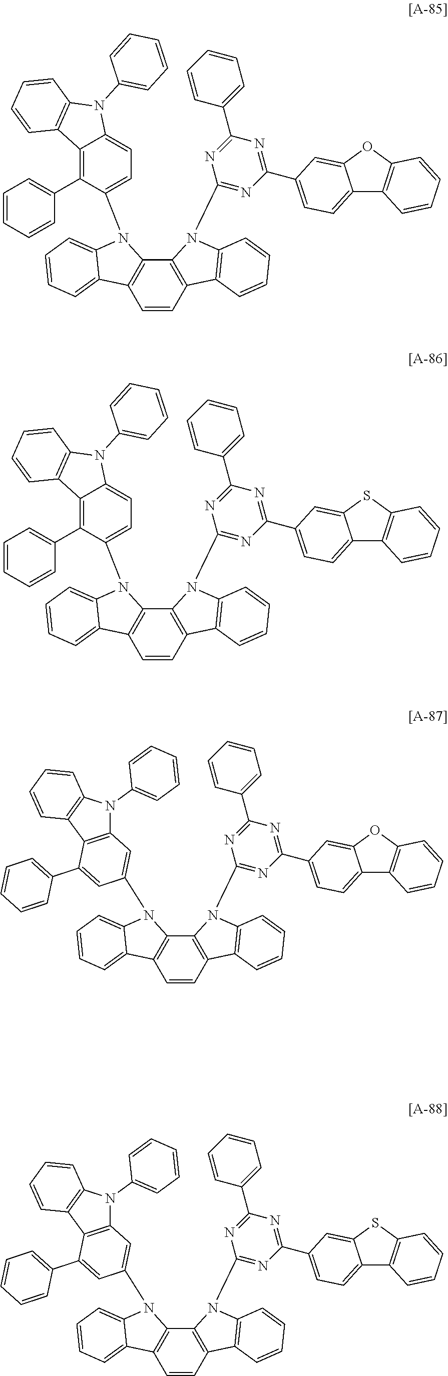 [SMILES: c1ccc(-c2nc(-c3ccc4c(c3)oc3ccccc34)nc(-n3c4ccccc4c4ccc5c6ccccc6n(-c6ccc7c(c6-c6ccccc6)c6ccccc6n7-c6ccccc6)c5c43)n2)cc1 ; c1ccc(-c2nc(-c3ccc4c(c3)sc3ccccc34)nc(-n3c4ccccc4c4ccc5c6ccccc6n(-c6ccc7c(c6-c6ccccc6)c6ccccc6n7-c6ccccc6)c5c43)n2)cc1 ; c1ccc(-c2nc(-c3ccc4c(c3)oc3ccccc34)nc(-n3c4ccccc4c4ccc5c6ccccc6n(-c6cc(-c7ccccc7)c7c8ccccc8n(-c8ccccc8)c7c6)c5c43)n2)cc1 ; c1ccc(-c2nc(-c3ccc4c(c3)sc3ccccc34)nc(-n3c4ccccc4c4ccc5c6ccccc6n(-c6cc(-c7ccccc7)c7c8ccccc8n(-c8ccccc8)c7c6)c5c43)n2)cc1]

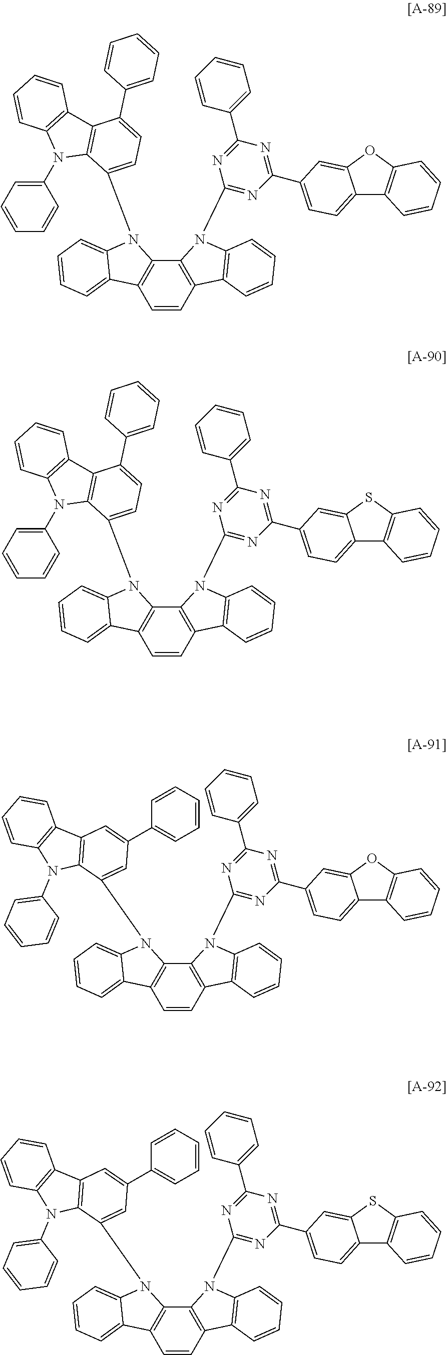 [SMILES: c1ccc(-c2nc(-c3ccc4c(c3)oc3ccccc34)nc(-n3c4ccccc4c4ccc5c6ccccc6n(-c6ccc(-c7ccccc7)c7c8ccccc8n(-c8ccccc8)c67)c5c43)n2)cc1 ; c1ccc(-c2nc(-c3ccc4c(c3)sc3ccccc34)nc(-n3c4ccccc4c4ccc5c6ccccc6n(-c6ccc(-c7ccccc7)c7c8ccccc8n(-c8ccccc8)c67)c5c43)n2)cc1 ; c1ccc(-c2cc(-n3c4ccccc4c4ccc5c6ccccc6n(-c6nc(-c7ccccc7)nc(-c7ccc8c(c7)oc7ccccc78)n6)c5c43)c3c(c2)c2ccccc2n3-c2ccccc2)cc1 ; c1ccc(-c2cc(-n3c4ccccc4c4ccc5c6ccccc6n(-c6nc(-c7ccccc7)nc(-c7ccc8c(c7)sc7ccccc78)n6)c5c43)c3c(c2)c2ccccc2n3-c2ccccc2)cc1]

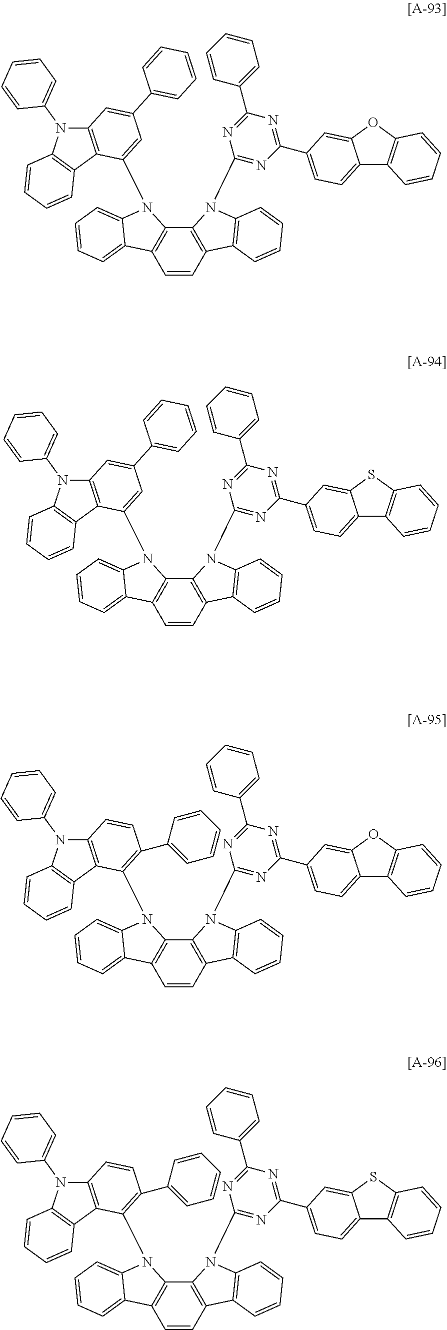 [SMILES: c1ccc(-c2cc(-n3c4ccccc4c4ccc5c6ccccc6n(-c6nc(-c7ccccc7)nc(-c7ccc8c(c7)oc7ccccc78)n6)c5c43)c3c4ccccc4n(-c4ccccc4)c3c2)cc1 ; c1ccc(-c2cc(-n3c4ccccc4c4ccc5c6ccccc6n(-c6nc(-c7ccccc7)nc(-c7ccc8c(c7)sc7ccccc78)n6)c5c43)c3c4ccccc4n(-c4ccccc4)c3c2)cc1 ; c1ccc(-c2nc(-c3ccc4c(c3)oc3ccccc34)nc(-n3c4ccccc4c4ccc5c6ccccc6n(-c6c(-c7ccccc7)ccc7c6c6ccccc6n7-c6ccccc6)c5c43)n2)cc1 ; c1ccc(-c2nc(-c3ccc4c(c3)sc3ccccc34)nc(-n3c4ccccc4c4ccc5c6ccccc6n(-c6c(-c7ccccc7)ccc7c6c6ccccc6n7-c6ccccc6)c5c43)n2)cc1]

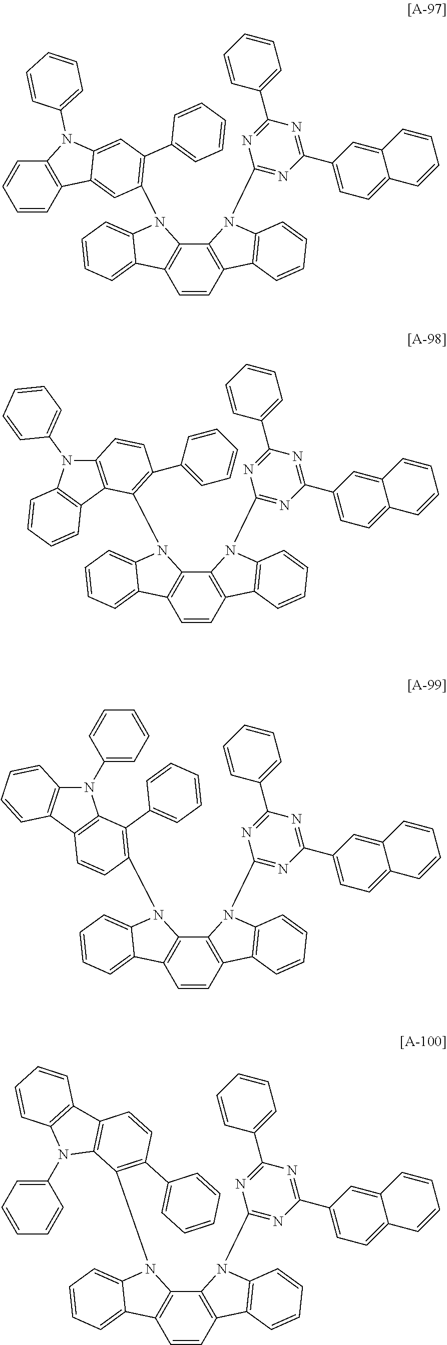 [SMILES: c1ccc(-c2nc(-c3ccc4ccccc4c3)nc(-n3c4ccccc4c4ccc5c6ccccc6n(-c6cc7c8ccccc8n(-c8ccccc8)c7cc6-c6ccccc6)c5c43)n2)cc1 ; c1ccc(-c2nc(-c3ccc4ccccc4c3)nc(-n3c4ccccc4c4ccc5c6ccccc6n(-c6c(-c7ccccc7)ccc7c6c6ccccc6n7-c6ccccc6)c5c43)n2)cc1 ; c1ccc(-c2nc(-c3ccc4ccccc4c3)nc(-n3c4ccccc4c4ccc5c6ccccc6n(-c6ccc7c8ccccc8n(-c8ccccc8)c7c6-c6ccccc6)c5c43)n2)cc1 ; c1ccc(-c2nc(-c3ccc4ccccc4c3)nc(-n3c4ccccc4c4ccc5c6ccccc6n(-c6c(-c7ccccc7)ccc7c8ccccc8n(-c8ccccc8)c67)c5c43)n2)cc1]

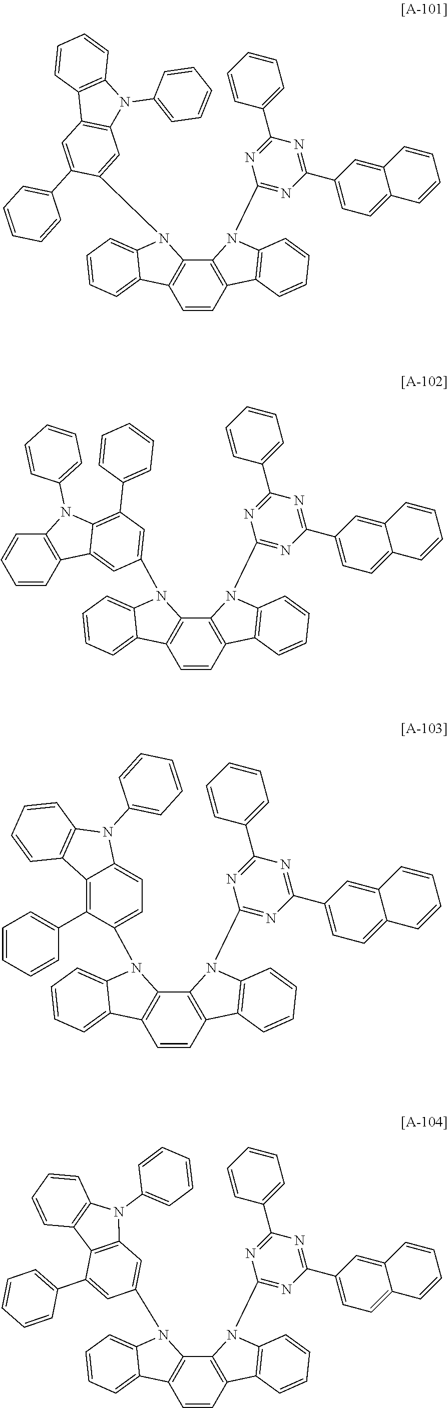 [SMILES: c1ccc(-c2nc(-c3ccc4ccccc4c3)nc(-n3c4ccccc4c4ccc5c6ccccc6n(-c6cc7c(cc6-c6ccccc6)c6ccccc6n7-c6ccccc6)c5c43)n2)cc1 ; c1ccc(-c2nc(-c3ccc4ccccc4c3)nc(-n3c4ccccc4c4ccc5c6ccccc6n(-c6cc(-c7ccccc7)c7c(c6)c6ccccc6n7-c6ccccc6)c5c43)n2)cc1 ; c1ccc(-c2nc(-c3ccc4ccccc4c3)nc(-n3c4ccccc4c4ccc5c6ccccc6n(-c6ccc7c(c6-c6ccccc6)c6ccccc6n7-c6ccccc6)c5c43)n2)cc1 ; c1ccc(-c2nc(-c3ccc4ccccc4c3)nc(-n3c4ccccc4c4ccc5c6ccccc6n(-c6cc(-c7ccccc7)c7c8ccccc8n(-c8ccccc8)c7c6)c5c43)n2)cc1]

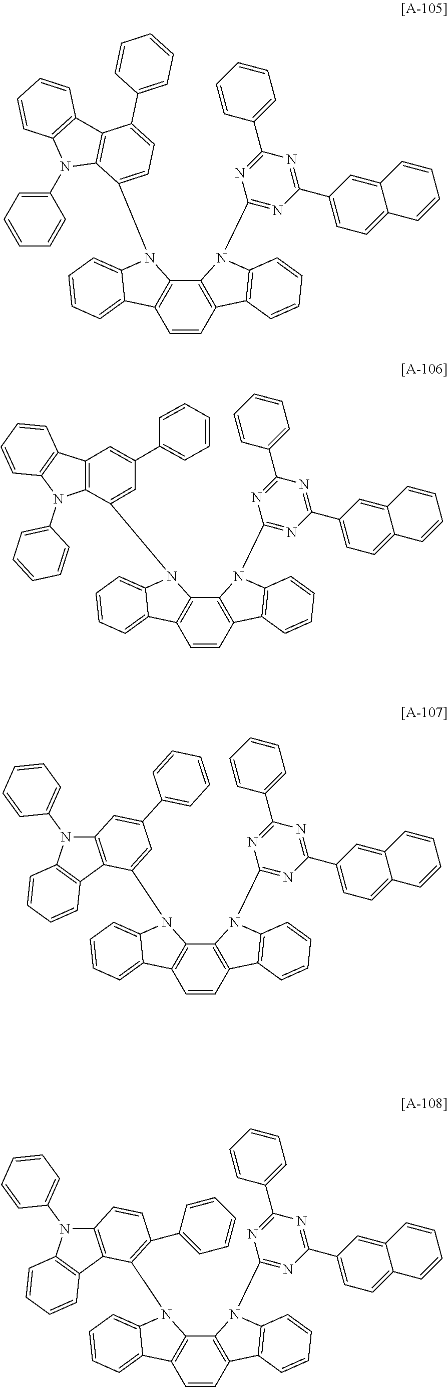 [SMILES: c1ccc(-c2nc(-c3ccc4ccccc4c3)nc(-n3c4ccccc4c4ccc5c6ccccc6n(-c6ccc(-c7ccccc7)c7c8ccccc8n(-c8ccccc8)c67)c5c43)n2)cc1 ; c1ccc(-c2cc(-n3c4ccccc4c4ccc5c6ccccc6n(-c6nc(-c7ccccc7)nc(-c7ccc8ccccc8c7)n6)c5c43)c3c(c2)c2ccccc2n3-c2ccccc2)cc1 ; c1ccc(-c2cc(-n3c4ccccc4c4ccc5c6ccccc6n(-c6nc(-c7ccccc7)nc(-c7ccc8ccccc8c7)n6)c5c43)c3c4ccccc4n(-c4ccccc4)c3c2)cc1 ; c1ccc(-c2nc(-c3ccc4ccccc4c3)nc(-n3c4ccccc4c4ccc5c6ccccc6n(-c6c(-c7ccccc7)ccc7c6c6ccccc6n7-c6ccccc6)c5c43)n2)cc1]

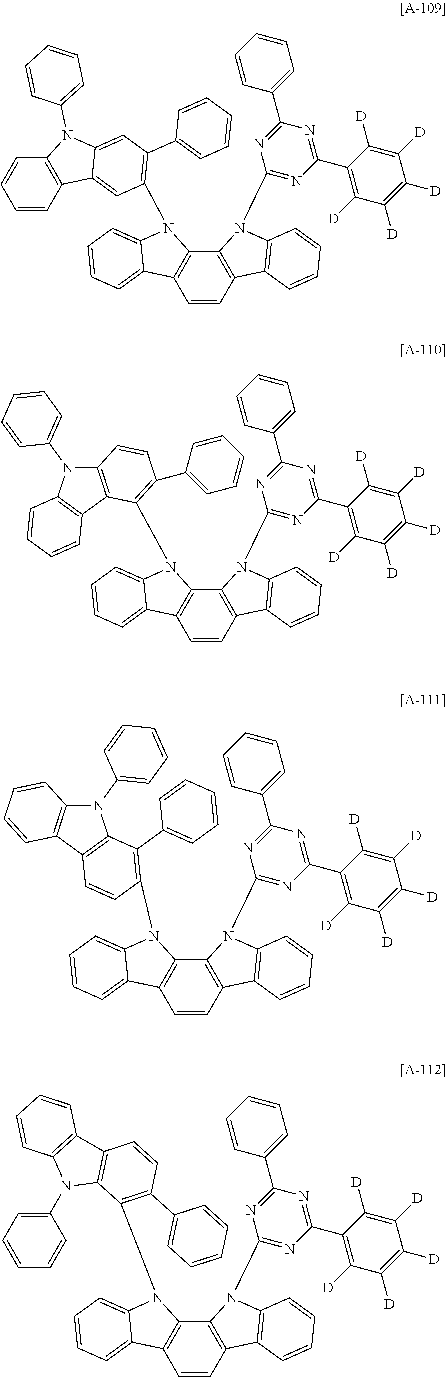

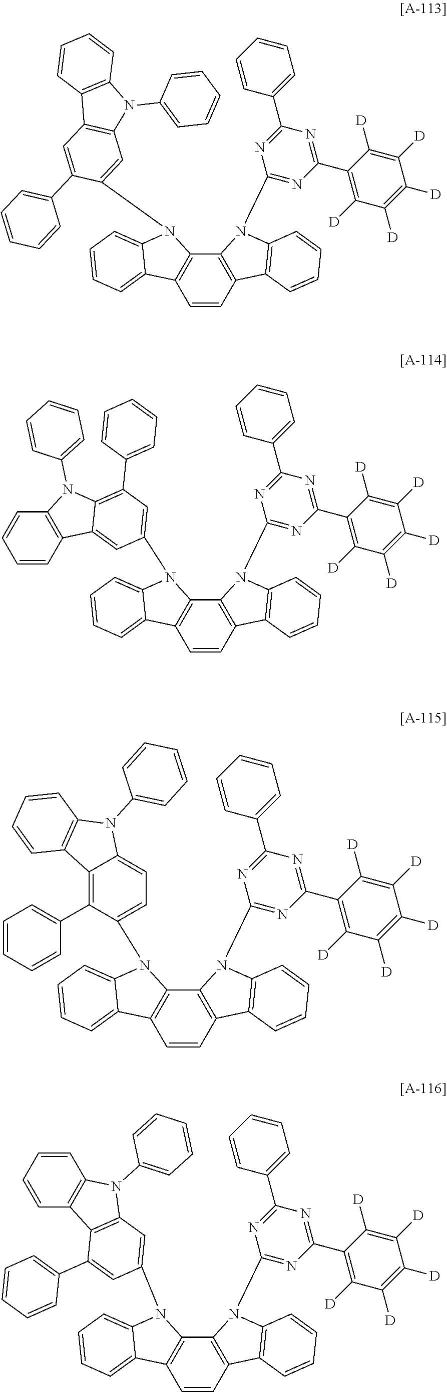

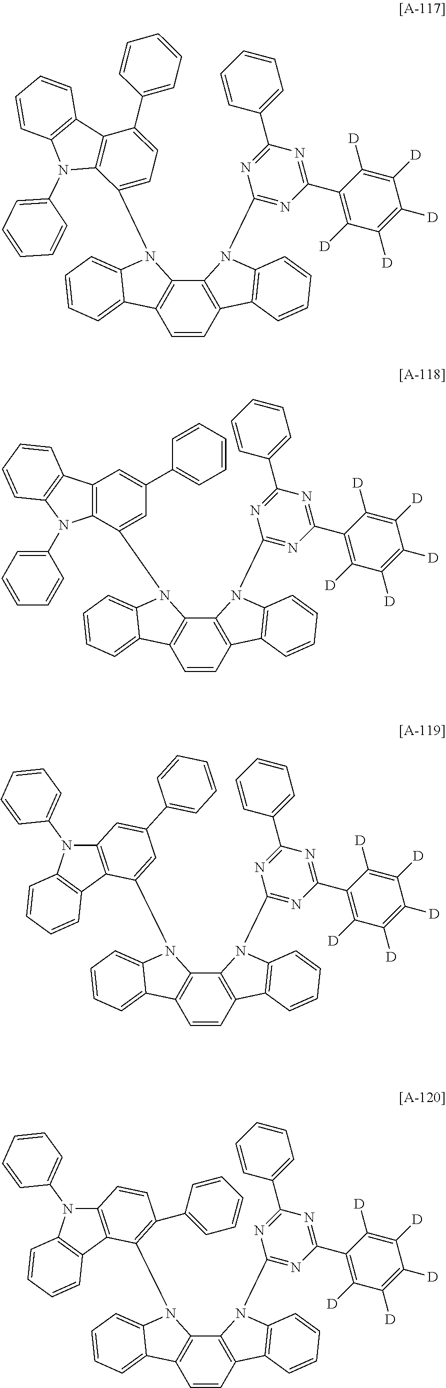

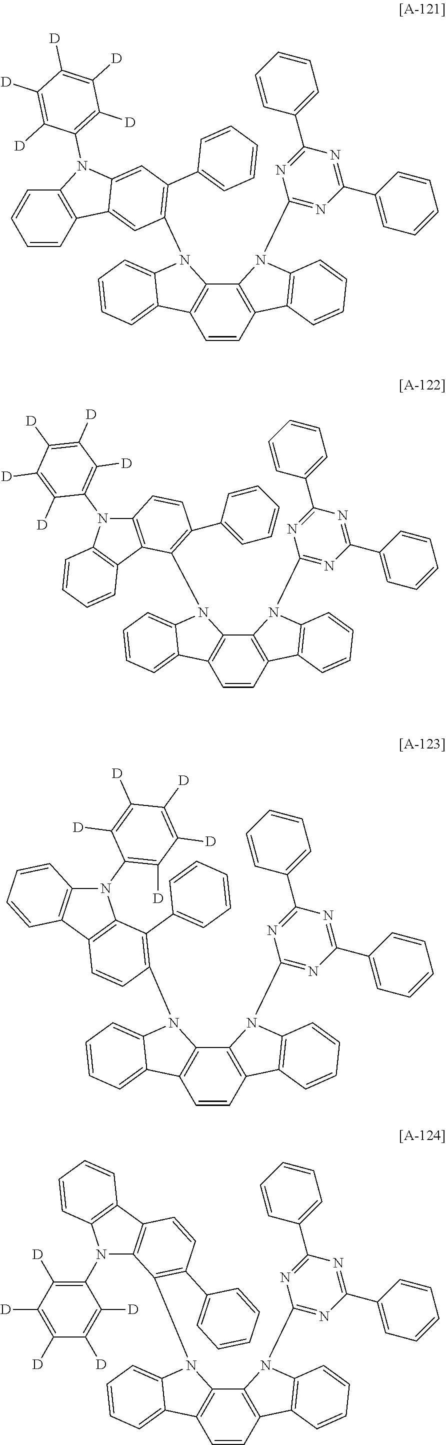

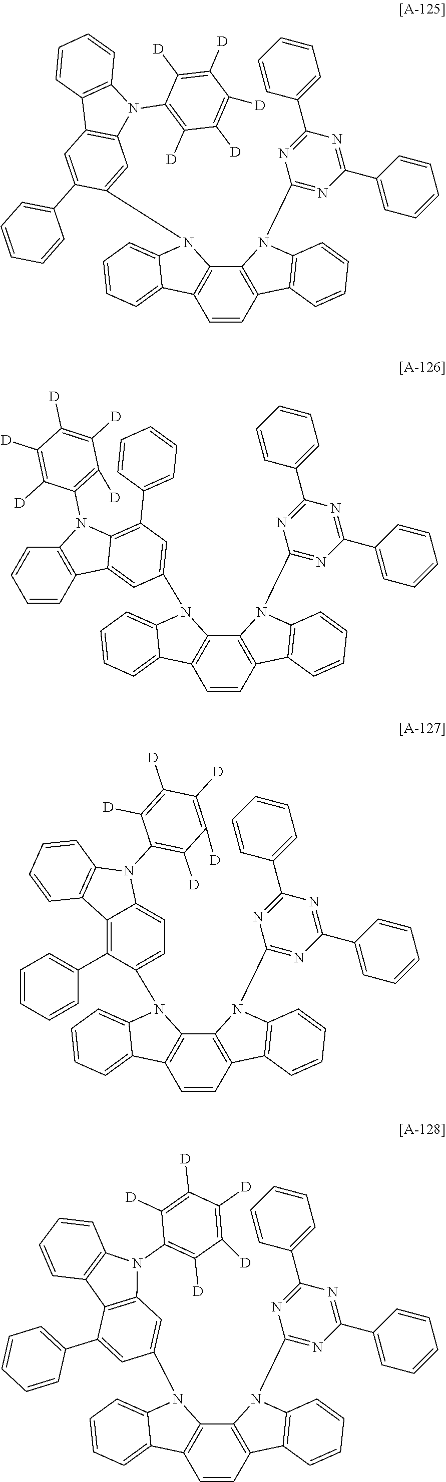

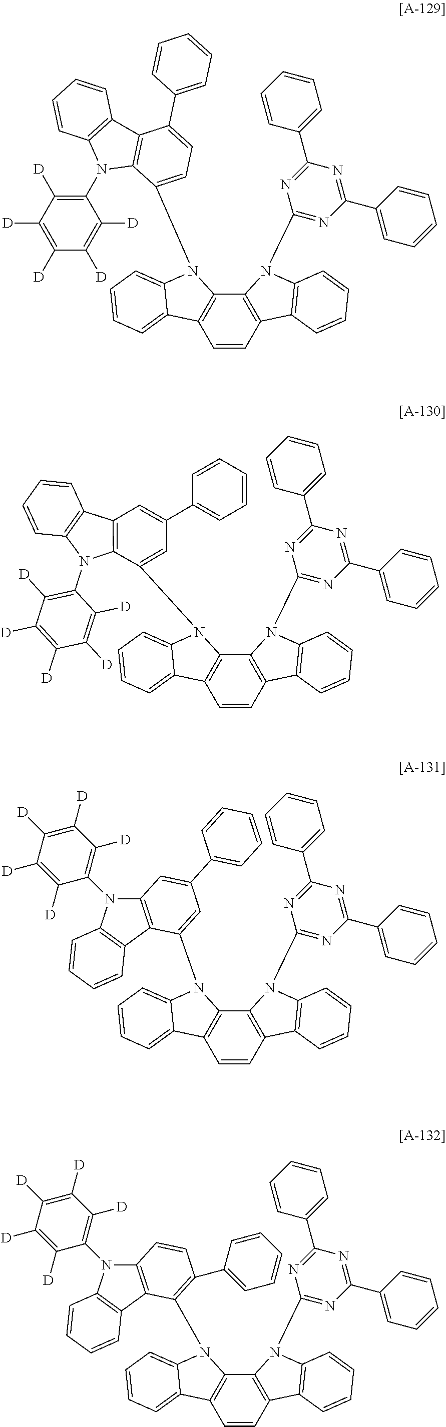

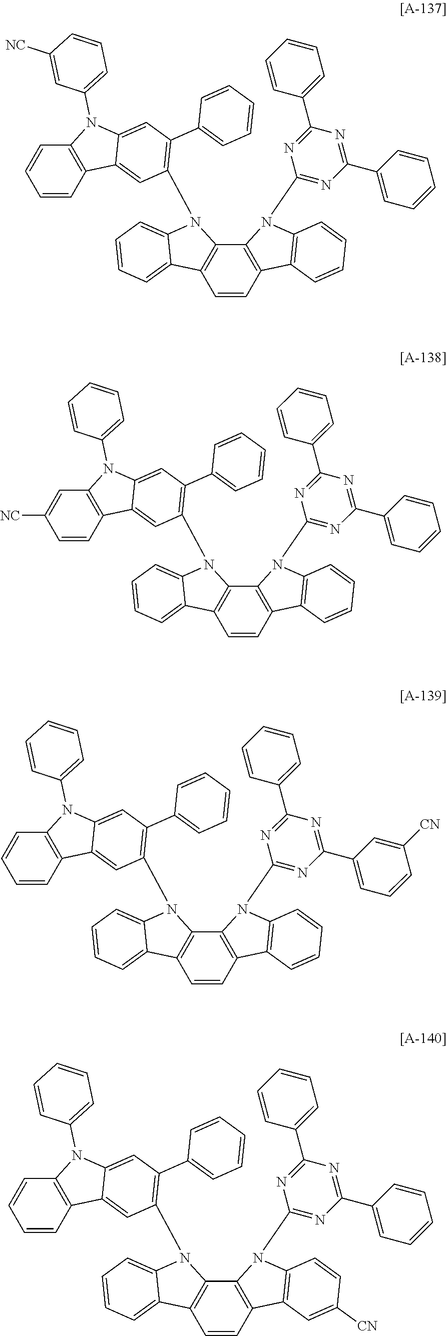 [SMILES: N#Cc1cccc(-n2c3ccccc3c3cc(-n4c5ccccc5c5ccc6c7ccccc7n(-c7nc(-c8ccccc8)nc(-c8ccccc8)n7)c6c54)c(-c4ccccc4)cc32)c1 ; N#Cc1ccc2c3cc(-n4c5ccccc5c5ccc6c7ccccc7n(-c7nc(-c8ccccc8)nc(-c8ccccc8)n7)c6c54)c(-c4ccccc4)cc3n(-c3ccccc3)c2c1 ; N#Cc1cccc(-c2nc(-c3ccccc3)nc(-n3c4ccccc4c4ccc5c6ccccc6n(-c6cc7c8ccccc8n(-c8ccccc8)c7cc6-c6ccccc6)c5c43)n2)c1 ; N#Cc1ccc2c(c1)c1ccc3c4ccccc4n(-c4cc5c6ccccc6n(-c6ccccc6)c5cc4-c4ccccc4)c3c1n2-c1nc(-c2ccccc2)nc(-c2ccccc2)n1]

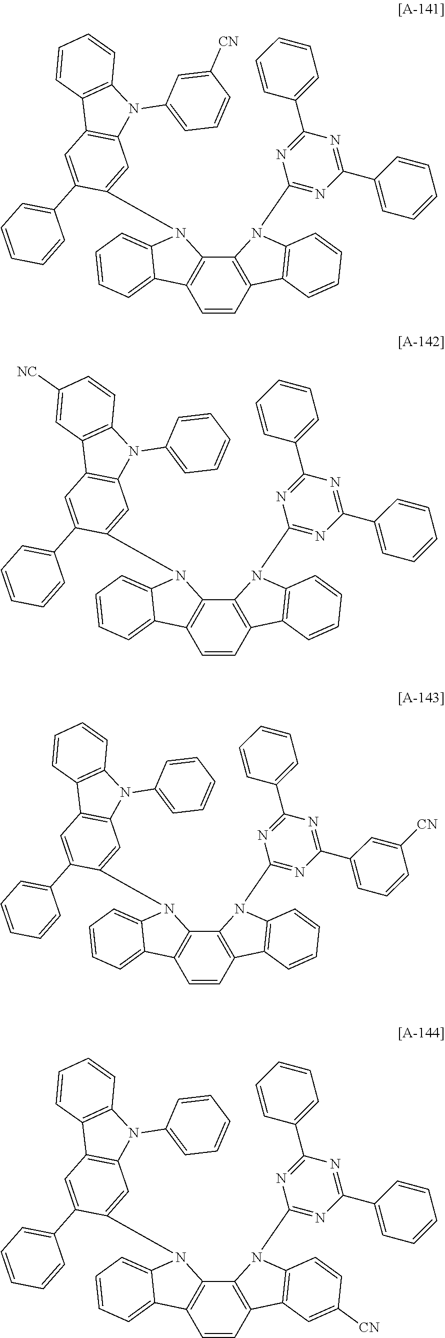 [SMILES: N#Cc1cccc(-n2c3ccccc3c3cc(-c4ccccc4)c(-n4c5ccccc5c5ccc6c7ccccc7n(-c7nc(-c8ccccc8)nc(-c8ccccc8)n7)c6c54)cc32)c1 ; N#Cc1ccc2c(c1)c1cc(-c3ccccc3)c(-n3c4ccccc4c4ccc5c6ccccc6n(-c6nc(-c7ccccc7)nc(-c7ccccc7)n6)c5c43)cc1n2-c1ccccc1 ; N#Cc1cccc(-c2nc(-c3ccccc3)nc(-n3c4ccccc4c4ccc5c6ccccc6n(-c6cc7c(cc6-c6ccccc6)c6ccccc6n7-c6ccccc6)c5c43)n2)c1 ; N#Cc1ccc2c(c1)c1ccc3c4ccccc4n(-c4cc5c(cc4-c4ccccc4)c4ccccc4n5-c4ccccc4)c3c1n2-c1nc(-c2ccccc2)nc(-c2ccccc2)n1]

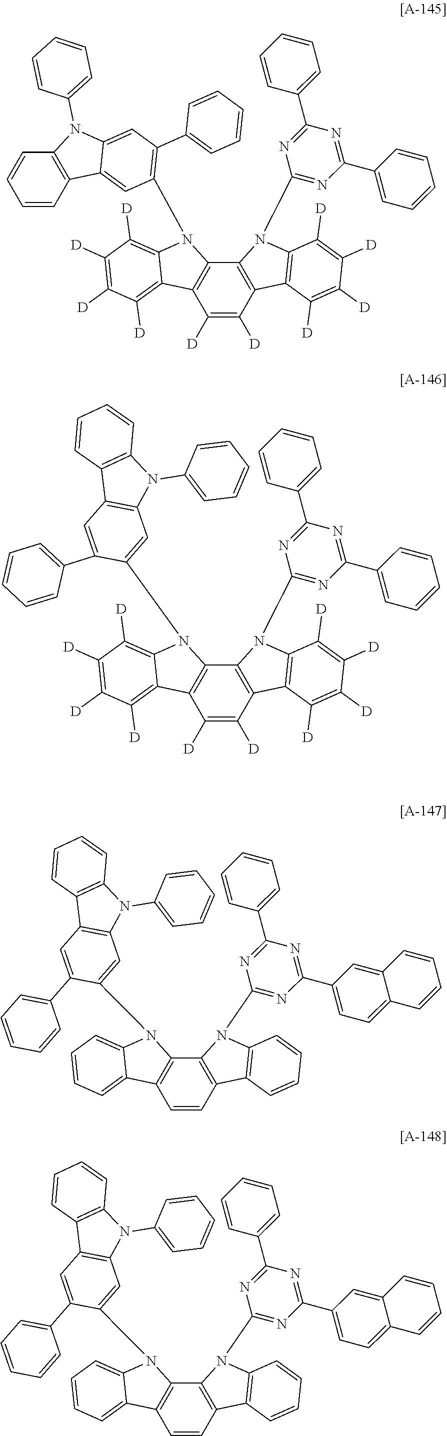

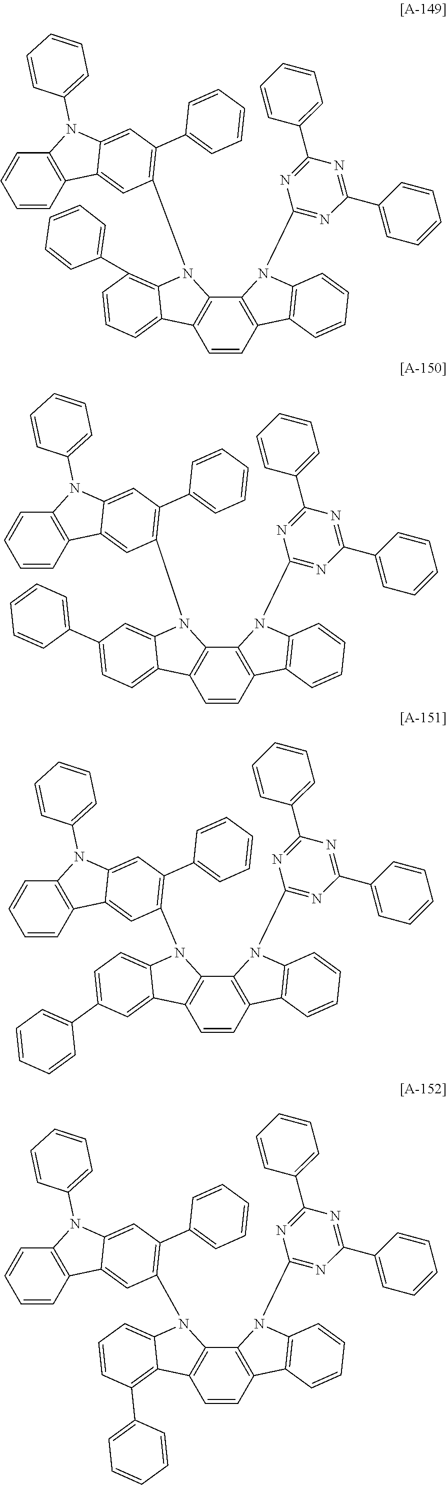 [SMILES: c1ccc(-c2nc(-c3ccccc3)nc(-n3c4ccccc4c4ccc5c6cccc(-c7ccccc7)c6n(-c6cc7c8ccccc8n(-c8ccccc8)c7cc6-c6ccccc6)c5c43)n2)cc1 ; c1ccc(-c2ccc3c4ccc5c6ccccc6n(-c6nc(-c7ccccc7)nc(-c7ccccc7)n6)c5c4n(-c4cc5c6ccccc6n(-c6ccccc6)c5cc4-c4ccccc4)c3c2)cc1 ; c1ccc(-c2ccc3c(c2)c2ccc4c5ccccc5n(-c5nc(-c6ccccc6)nc(-c6ccccc6)n5)c4c2n3-c2cc3c4ccccc4n(-c4ccccc4)c3cc2-c2ccccc2)cc1 ; c1ccc(-c2nc(-c3ccccc3)nc(-n3c4ccccc4c4ccc5c6c(-c7ccccc7)cccc6n(-c6cc7c8ccccc8n(-c8ccccc8)c7cc6-c6ccccc6)c5c43)n2)cc1]

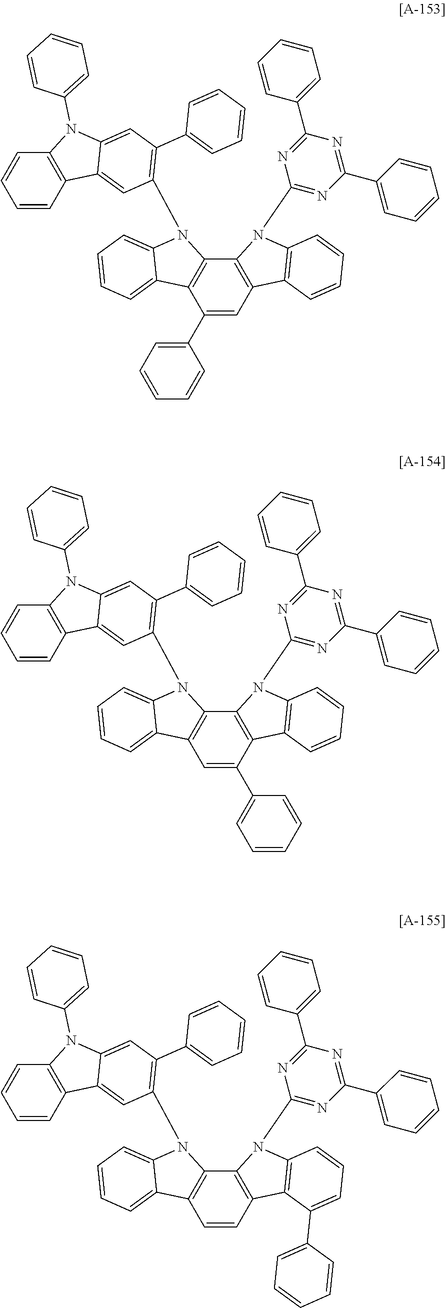 [SMILES: c1ccc(-c2nc(-c3ccccc3)nc(-n3c4ccccc4c4cc(-c5ccccc5)c5c6ccccc6n(-c6cc7c8ccccc8n(-c8ccccc8)c7cc6-c6ccccc6)c5c43)n2)cc1 ; c1ccc(-c2nc(-c3ccccc3)nc(-n3c4ccccc4c4c(-c5ccccc5)cc5c6ccccc6n(-c6cc7c8ccccc8n(-c8ccccc8)c7cc6-c6ccccc6)c5c43)n2)cc1 ; c1ccc(-c2nc(-c3ccccc3)nc(-n3c4cccc(-c5ccccc5)c4c4ccc5c6ccccc6n(-c6cc7c8ccccc8n(-c8ccccc8)c7cc6-c6ccccc6)c5c43)n2)cc1]

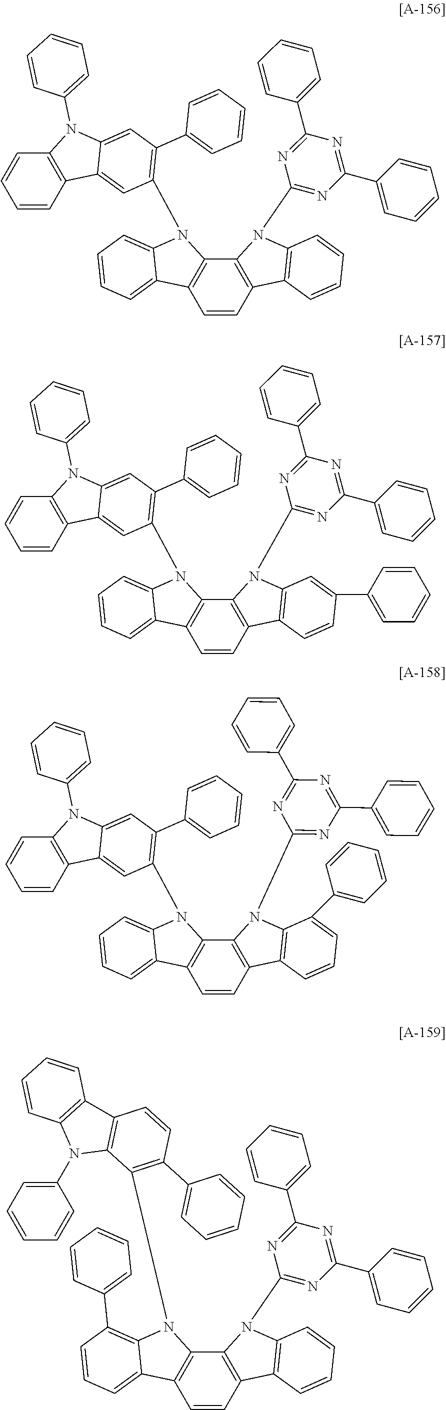 [SMILES: c1ccc(-c2nc(-c3ccccc3)nc(-n3c4ccccc4c4ccc5c6ccccc6n(-c6cc7c8ccccc8n(-c8ccccc8)c7cc6-c6ccccc6)c5c43)n2)cc1 ; c1ccc(-c2ccc3c4ccc5c6ccccc6n(-c6cc7c8ccccc8n(-c8ccccc8)c7cc6-c6ccccc6)c5c4n(-c4nc(-c5ccccc5)nc(-c5ccccc5)n4)c3c2)cc1 ; c1ccc(-c2nc(-c3ccccc3)nc(-n3c4c(-c5ccccc5)cccc4c4ccc5c6ccccc6n(-c6cc7c8ccccc8n(-c8ccccc8)c7cc6-c6ccccc6)c5c43)n2)cc1 ; c1ccc(-c2nc(-c3ccccc3)nc(-n3c4ccccc4c4ccc5c6cccc(-c7ccccc7)c6n(-c6c(-c7ccccc7)ccc7c8ccccc8n(-c8ccccc8)c67)c5c43)n2)cc1]

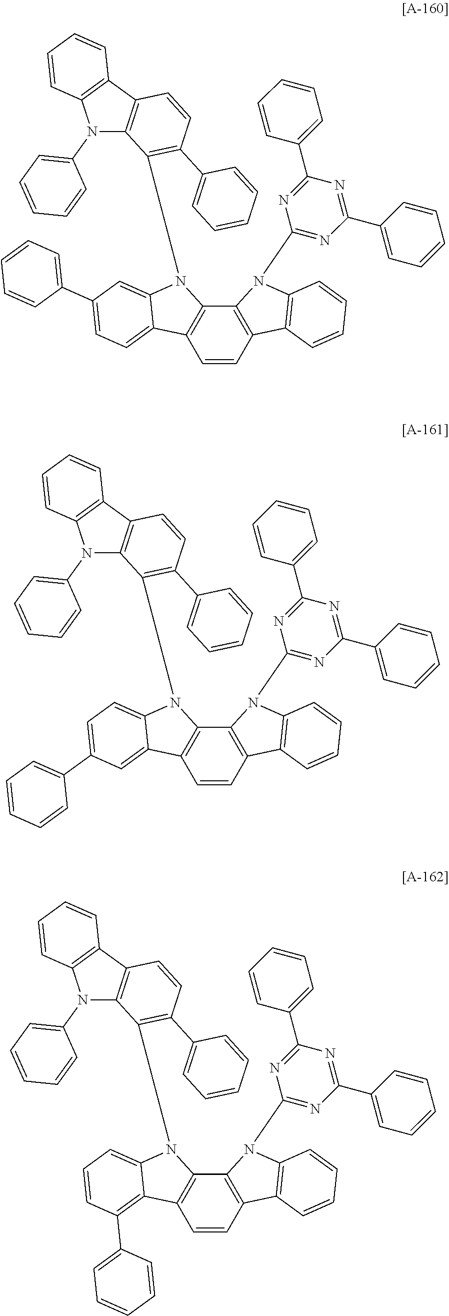 [SMILES: c1ccc(-c2ccc3c4ccc5c6ccccc6n(-c6nc(-c7ccccc7)nc(-c7ccccc7)n6)c5c4n(-c4c(-c5ccccc5)ccc5c6ccccc6n(-c6ccccc6)c45)c3c2)cc1 ; c1ccc(-c2ccc3c(c2)c2ccc4c5ccccc5n(-c5nc(-c6ccccc6)nc(-c6ccccc6)n5)c4c2n3-c2c(-c3ccccc3)ccc3c4ccccc4n(-c4ccccc4)c23)cc1 ; c1ccc(-c2nc(-c3ccccc3)nc(-n3c4ccccc4c4ccc5c6c(-c7ccccc7)cccc6n(-c6c(-c7ccccc7)ccc7c8ccccc8n(-c8ccccc8)c67)c5c43)n2)cc1]

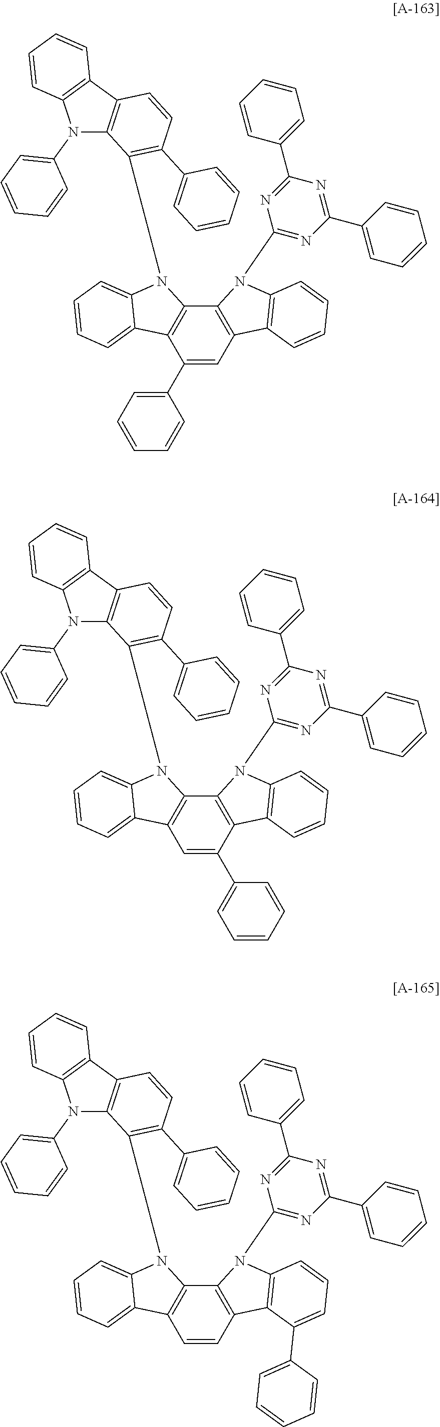 [SMILES: c1ccc(-c2nc(-c3ccccc3)nc(-n3c4ccccc4c4cc(-c5ccccc5)c5c6ccccc6n(-c6c(-c7ccccc7)ccc7c8ccccc8n(-c8ccccc8)c67)c5c43)n2)cc1 ; c1ccc(-c2nc(-c3ccccc3)nc(-n3c4ccccc4c4c(-c5ccccc5)cc5c6ccccc6n(-c6c(-c7ccccc7)ccc7c8ccccc8n(-c8ccccc8)c67)c5c43)n2)cc1 ; c1ccc(-c2nc(-c3ccccc3)nc(-n3c4cccc(-c5ccccc5)c4c4ccc5c6ccccc6n(-c6c(-c7ccccc7)ccc7c8ccccc8n(-c8ccccc8)c67)c5c43)n2)cc1]

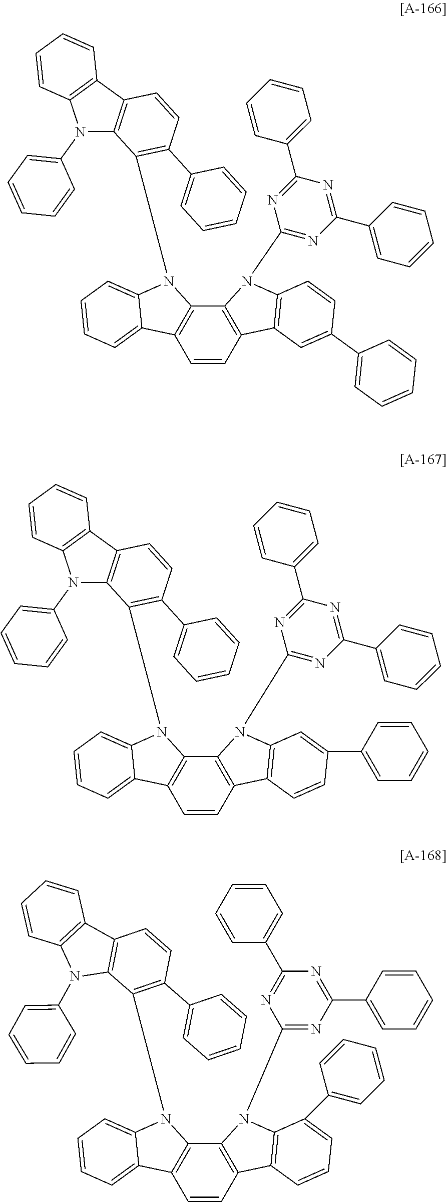 [SMILES: c1ccc(-c2ccc3c(c2)c2ccc4c5ccccc5n(-c5c(-c6ccccc6)ccc6c7ccccc7n(-c7ccccc7)c56)c4c2n3-c2nc(-c3ccccc3)nc(-c3ccccc3)n2)cc1 ; c1ccc(-c2ccc3c4ccc5c6ccccc6n(-c6c(-c7ccccc7)ccc7c8ccccc8n(-c8ccccc8)c67)c5c4n(-c4nc(-c5ccccc5)nc(-c5ccccc5)n4)c3c2)cc1 ; c1ccc(-c2nc(-c3ccccc3)nc(-n3c4c(-c5ccccc5)cccc4c4ccc5c6ccccc6n(-c6c(-c7ccccc7)ccc7c8ccccc8n(-c8ccccc8)c67)c5c43)n2)cc1]

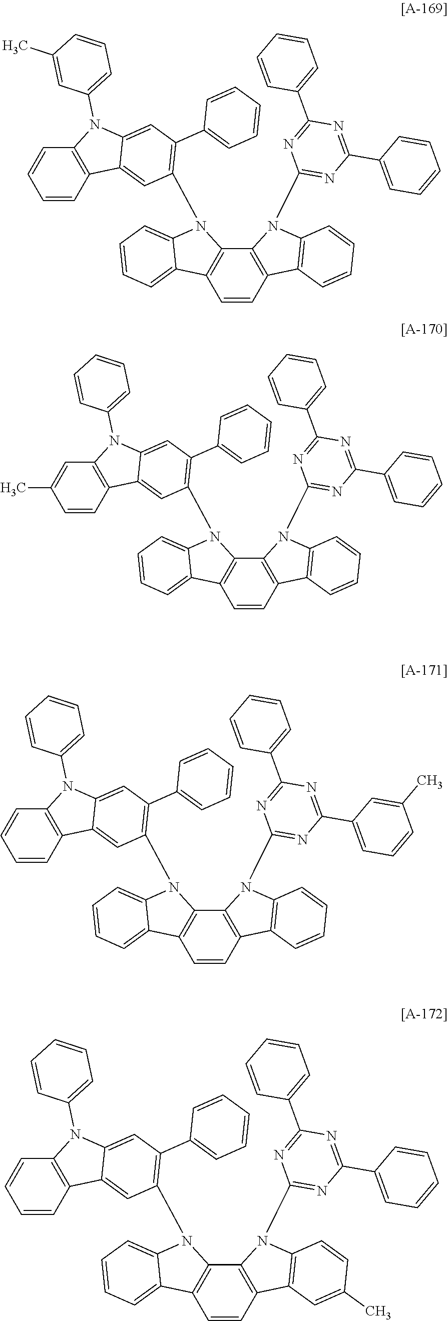 [SMILES: Cc1cccc(-n2c3ccccc3c3cc(-n4c5ccccc5c5ccc6c7ccccc7n(-c7nc(-c8ccccc8)nc(-c8ccccc8)n7)c6c54)c(-c4ccccc4)cc32)c1 ; Cc1ccc2c3cc(-n4c5ccccc5c5ccc6c7ccccc7n(-c7nc(-c8ccccc8)nc(-c8ccccc8)n7)c6c54)c(-c4ccccc4)cc3n(-c3ccccc3)c2c1 ; Cc1cccc(-c2nc(-c3ccccc3)nc(-n3c4ccccc4c4ccc5c6ccccc6n(-c6cc7c8ccccc8n(-c8ccccc8)c7cc6-c6ccccc6)c5c43)n2)c1 ; Cc1ccc2c(c1)c1ccc3c4ccccc4n(-c4cc5c6ccccc6n(-c6ccccc6)c5cc4-c4ccccc4)c3c1n2-c1nc(-c2ccccc2)nc(-c2ccccc2)n1]

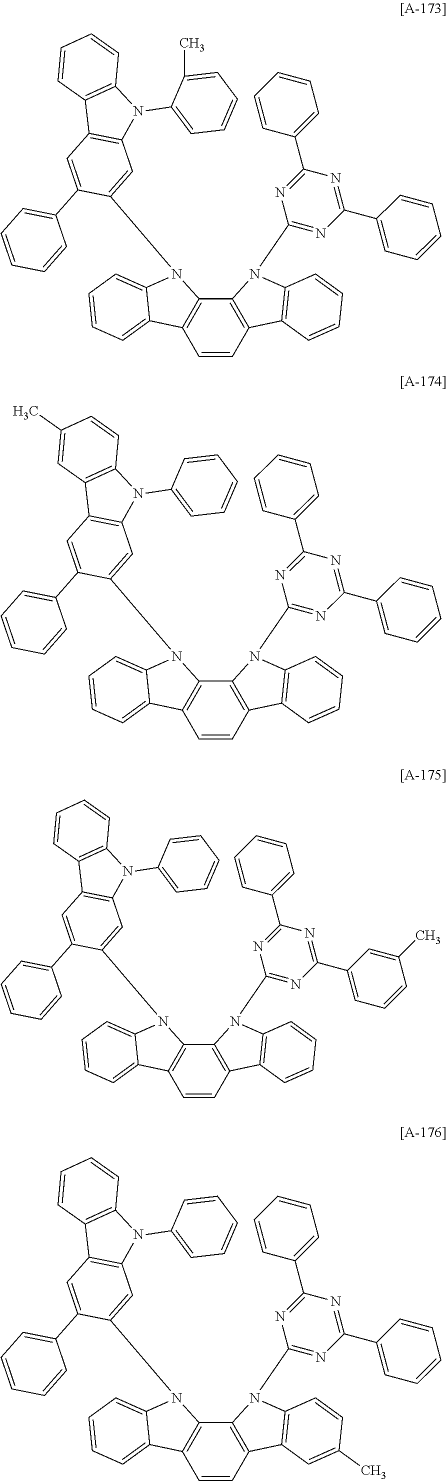 [SMILES: Cc1ccccc1-n1c2ccccc2c2cc(-c3ccccc3)c(-n3c4ccccc4c4ccc5c6ccccc6n(-c6nc(-c7ccccc7)nc(-c7ccccc7)n6)c5c43)cc21 ; Cc1ccc2c(c1)c1cc(-c3ccccc3)c(-n3c4ccccc4c4ccc5c6ccccc6n(-c6nc(-c7ccccc7)nc(-c7ccccc7)n6)c5c43)cc1n2-c1ccccc1 ; Cc1cccc(-c2nc(-c3ccccc3)nc(-n3c4ccccc4c4ccc5c6ccccc6n(-c6cc7c(cc6-c6ccccc6)c6ccccc6n7-c6ccccc6)c5c43)n2)c1 ; Cc1ccc2c(c1)c1ccc3c4ccccc4n(-c4cc5c(cc4-c4ccccc4)c4ccccc4n5-c4ccccc4)c3c1n2-c1nc(-c2ccccc2)nc(-c2ccccc2)n1]

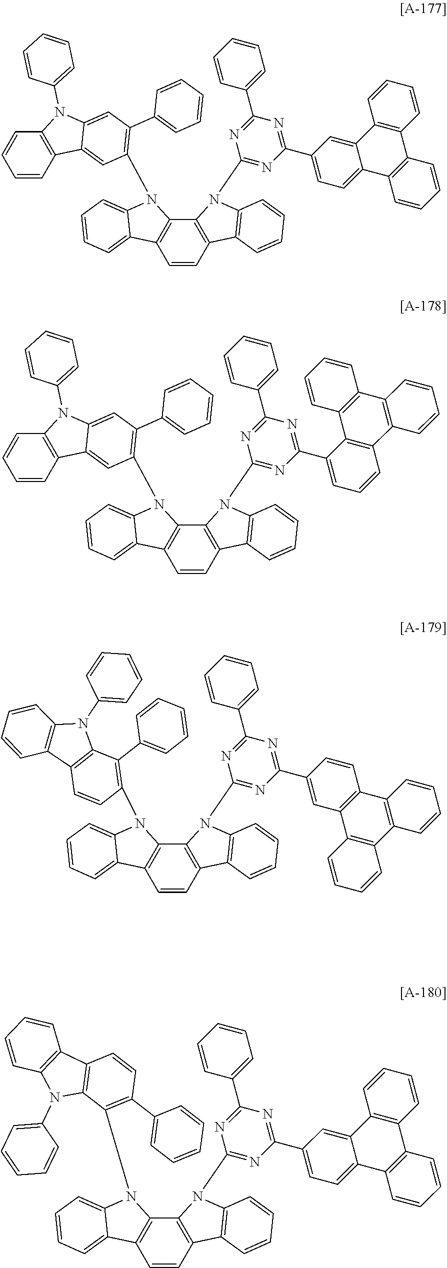

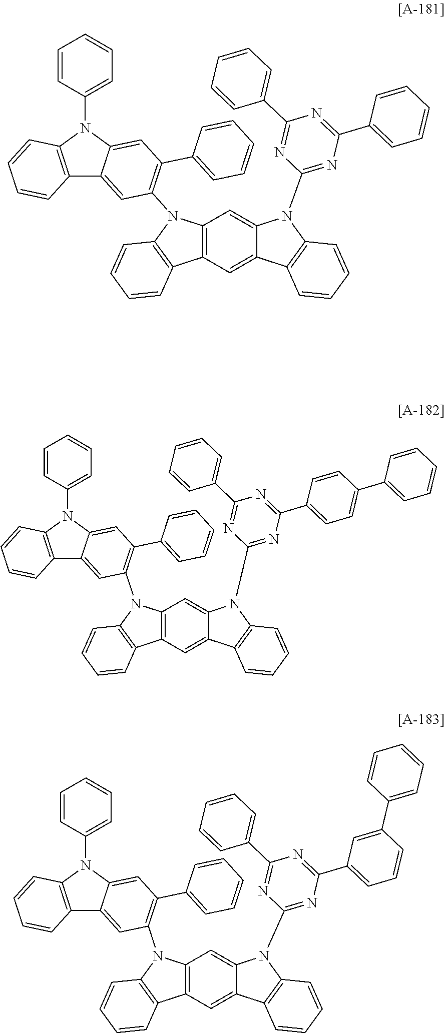 [SMILES: c1ccc(-c2nc(-c3ccccc3)nc(-n3c4ccccc4c4cc5c6ccccc6n(-c6cc7c8ccccc8n(-c8ccccc8)c7cc6-c6ccccc6)c5cc43)n2)cc1 ; c1ccc(-c2ccc(-c3nc(-c4ccccc4)nc(-n4c5ccccc5c5cc6c7ccccc7n(-c7cc8c9ccccc9n(-c9ccccc9)c8cc7-c7ccccc7)c6cc54)n3)cc2)cc1 ; c1ccc(-c2cccc(-c3nc(-c4ccccc4)nc(-n4c5ccccc5c5cc6c7ccccc7n(-c7cc8c9ccccc9n(-c9ccccc9)c8cc7-c7ccccc7)c6cc54)n3)c2)cc1]

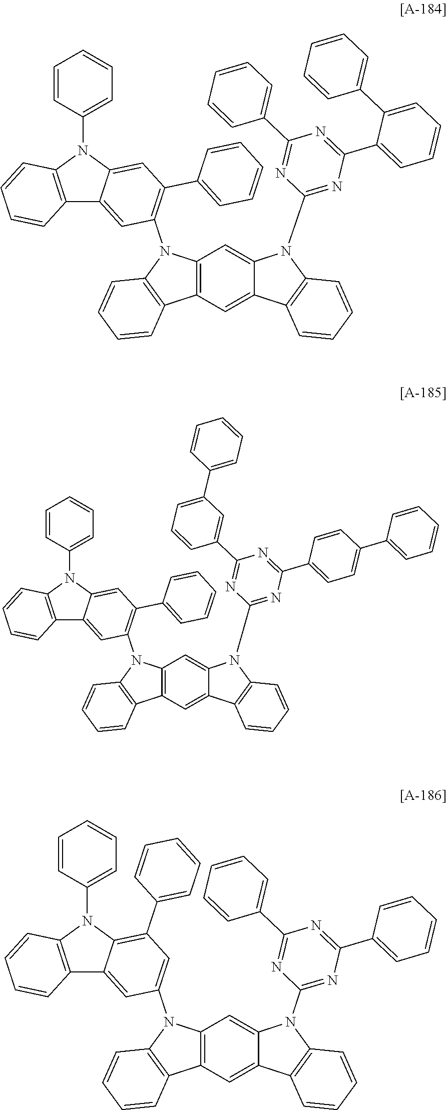 [SMILES: c1ccc(-c2nc(-c3ccccc3-c3ccccc3)nc(-n3c4ccccc4c4cc5c6ccccc6n(-c6cc7c8ccccc8n(-c8ccccc8)c7cc6-c6ccccc6)c5cc43)n2)cc1 ; c1ccc(-c2ccc(-c3nc(-c4cccc(-c5ccccc5)c4)nc(-n4c5ccccc5c5cc6c7ccccc7n(-c7cc8c9ccccc9n(-c9ccccc9)c8cc7-c7ccccc7)c6cc54)n3)cc2)cc1 ; c1ccc(-c2nc(-c3ccccc3)nc(-n3c4ccccc4c4cc5c6ccccc6n(-c6cc(-c7ccccc7)c7c(c6)c6ccccc6n7-c6ccccc6)c5cc43)n2)cc1]

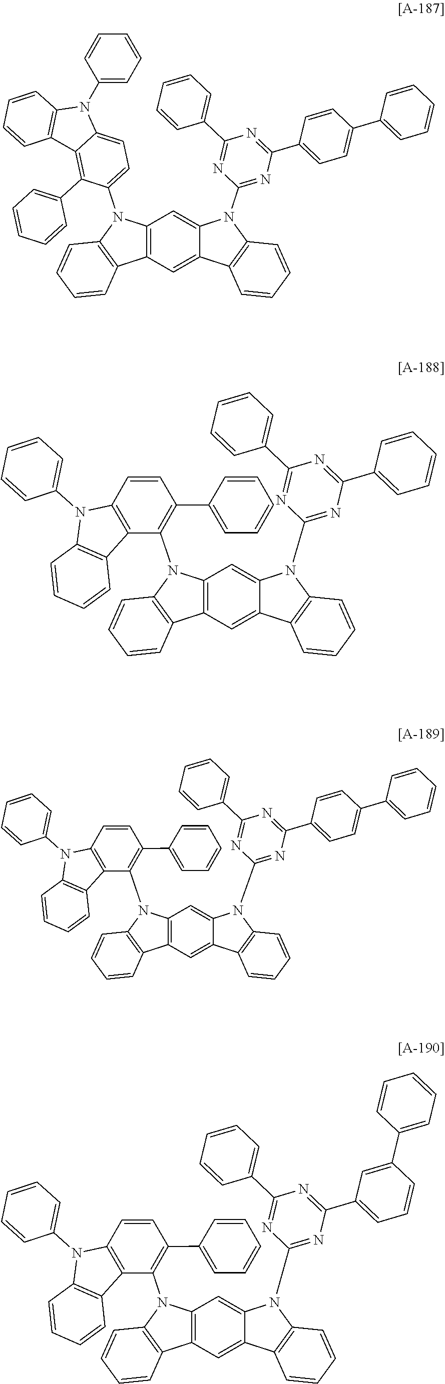 [SMILES: c1ccc(-c2ccc(-c3nc(-c4ccccc4)nc(-n4c5ccccc5c5cc6c7ccccc7n(-c7ccc8c(c7-c7ccccc7)c7ccccc7n8-c7ccccc7)c6cc54)n3)cc2)cc1 ; c1ccc(-c2nc(-c3ccccc3)nc(-n3c4ccccc4c4cc5c6ccccc6n(-c6c(-c7ccccc7)ccc7c6c6ccccc6n7-c6ccccc6)c5cc43)n2)cc1 ; c1ccc(-c2ccc(-c3nc(-c4ccccc4)nc(-n4c5ccccc5c5cc6c7ccccc7n(-c7c(-c8ccccc8)ccc8c7c7ccccc7n8-c7ccccc7)c6cc54)n3)cc2)cc1 ; c1ccc(-c2cccc(-c3nc(-c4ccccc4)nc(-n4c5ccccc5c5cc6c7ccccc7n(-c7c(-c8ccccc8)ccc8c7c7ccccc7n8-c7ccccc7)c6cc54)n3)c2)cc1]

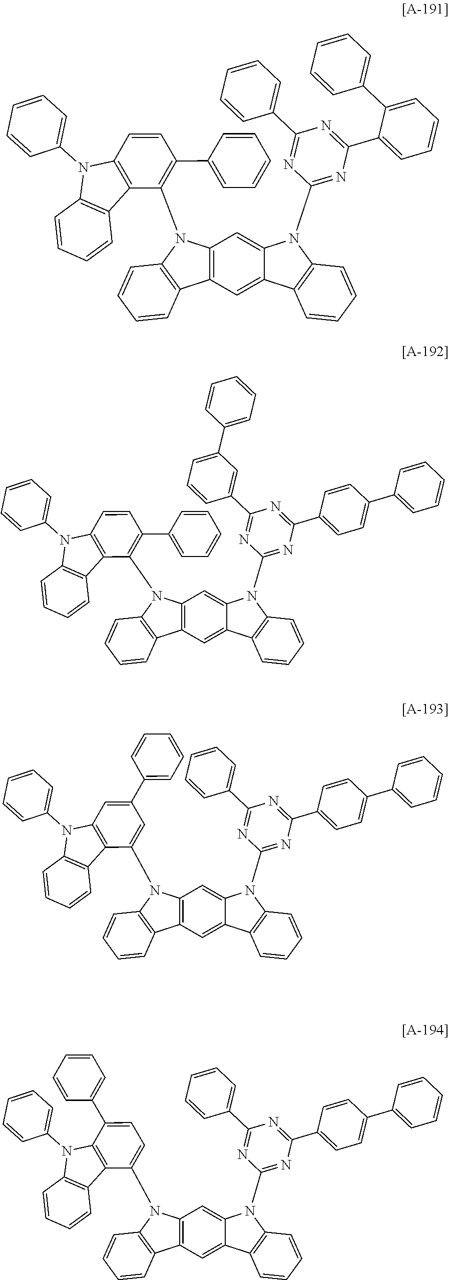 [SMILES: c1ccc(-c2nc(-c3ccccc3-c3ccccc3)nc(-n3c4ccccc4c4cc5c6ccccc6n(-c6c(-c7ccccc7)ccc7c6c6ccccc6n7-c6ccccc6)c5cc43)n2)cc1 ; c1ccc(-c2ccc(-c3nc(-c4cccc(-c5ccccc5)c4)nc(-n4c5ccccc5c5cc6c7ccccc7n(-c7c(-c8ccccc8)ccc8c7c7ccccc7n8-c7ccccc7)c6cc54)n3)cc2)cc1 ; c1ccc(-c2ccc(-c3nc(-c4ccccc4)nc(-n4c5ccccc5c5cc6c7ccccc7n(-c7cc(-c8ccccc8)cc8c7c7ccccc7n8-c7ccccc7)c6cc54)n3)cc2)cc1 ; c1ccc(-c2ccc(-c3nc(-c4ccccc4)nc(-n4c5ccccc5c5cc6c7ccccc7n(-c7ccc(-c8ccccc8)c8c7c7ccccc7n8-c7ccccc7)c6cc54)n3)cc2)cc1]

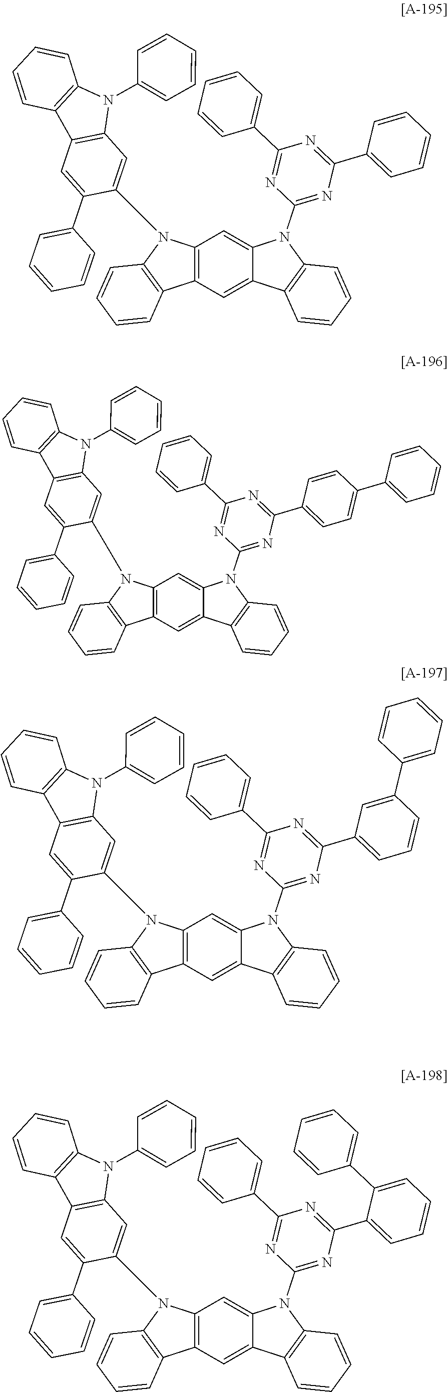 [SMILES: c1ccc(-c2nc(-c3ccccc3)nc(-n3c4ccccc4c4cc5c6ccccc6n(-c6cc7c(cc6-c6ccccc6)c6ccccc6n7-c6ccccc6)c5cc43)n2)cc1 ; c1ccc(-c2ccc(-c3nc(-c4ccccc4)nc(-n4c5ccccc5c5cc6c7ccccc7n(-c7cc8c(cc7-c7ccccc7)c7ccccc7n8-c7ccccc7)c6cc54)n3)cc2)cc1 ; c1ccc(-c2cccc(-c3nc(-c4ccccc4)nc(-n4c5ccccc5c5cc6c7ccccc7n(-c7cc8c(cc7-c7ccccc7)c7ccccc7n8-c7ccccc7)c6cc54)n3)c2)cc1 ; c1ccc(-c2nc(-c3ccccc3-c3ccccc3)nc(-n3c4ccccc4c4cc5c6ccccc6n(-c6cc7c(cc6-c6ccccc6)c6ccccc6n7-c6ccccc6)c5cc43)n2)cc1]

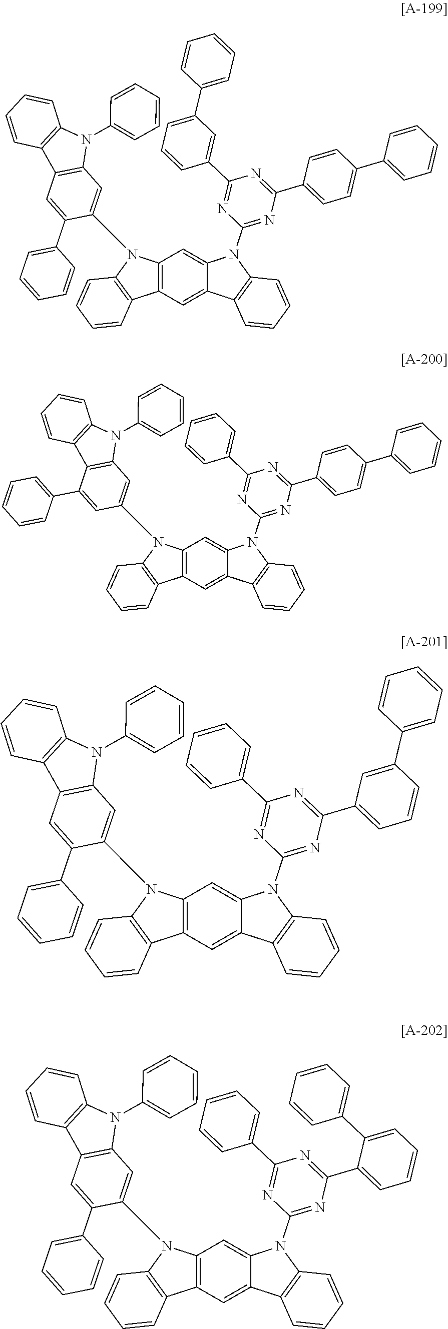 [SMILES: c1ccc(-c2ccc(-c3nc(-c4cccc(-c5ccccc5)c4)nc(-n4c5ccccc5c5cc6c7ccccc7n(-c7cc8c(cc7-c7ccccc7)c7ccccc7n8-c7ccccc7)c6cc54)n3)cc2)cc1 ; c1ccc(-c2ccc(-c3nc(-c4ccccc4)nc(-n4c5ccccc5c5cc6c7ccccc7n(-c7cc(-c8ccccc8)c8c9ccccc9n(-c9ccccc9)c8c7)c6cc54)n3)cc2)cc1 ; c1ccc(-c2cccc(-c3nc(-c4ccccc4)nc(-n4c5ccccc5c5cc6c7ccccc7n(-c7cc8c(cc7-c7ccccc7)c7ccccc7n8-c7ccccc7)c6cc54)n3)c2)cc1 ; c1ccc(-c2nc(-c3ccccc3-c3ccccc3)nc(-n3c4ccccc4c4cc5c6ccccc6n(-c6cc7c(cc6-c6ccccc6)c6ccccc6n7-c6ccccc6)c5cc43)n2)cc1]

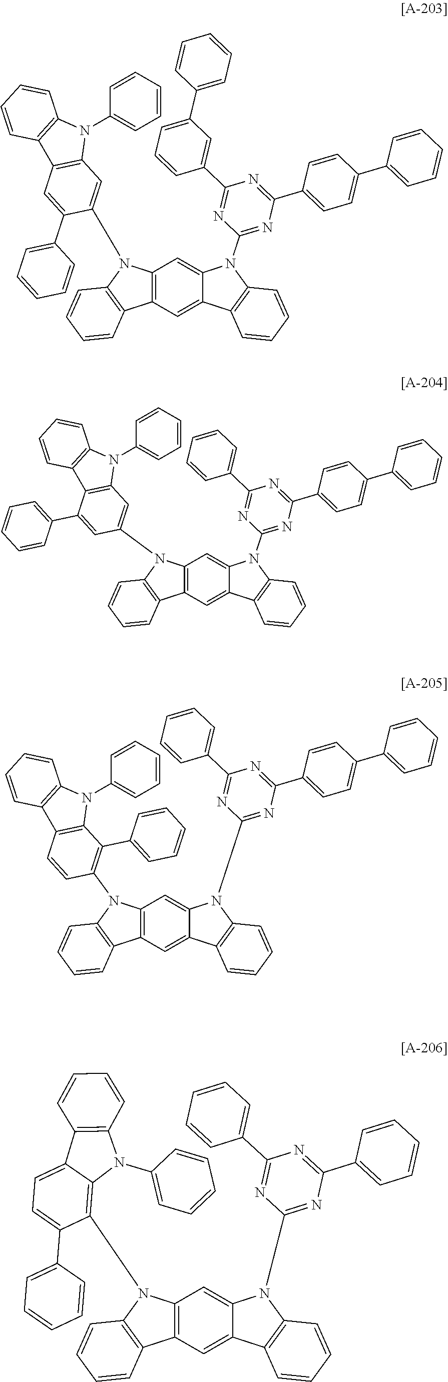 [SMILES: c1ccc(-c2ccc(-c3nc(-c4cccc(-c5ccccc5)c4)nc(-n4c5ccccc5c5cc6c7ccccc7n(-c7cc8c(cc7-c7ccccc7)c7ccccc7n8-c7ccccc7)c6cc54)n3)cc2)cc1 ; c1ccc(-c2ccc(-c3nc(-c4ccccc4)nc(-n4c5ccccc5c5cc6c7ccccc7n(-c7cc(-c8ccccc8)c8c9ccccc9n(-c9ccccc9)c8c7)c6cc54)n3)cc2)cc1 ; c1ccc(-c2ccc(-c3nc(-c4ccccc4)nc(-n4c5ccccc5c5cc6c7ccccc7n(-c7ccc8c9ccccc9n(-c9ccccc9)c8c7-c7ccccc7)c6cc54)n3)cc2)cc1 ; c1ccc(-c2nc(-c3ccccc3)nc(-n3c4ccccc4c4cc5c6ccccc6n(-c6c(-c7ccccc7)ccc7c8ccccc8n(-c8ccccc8)c67)c5cc43)n2)cc1]

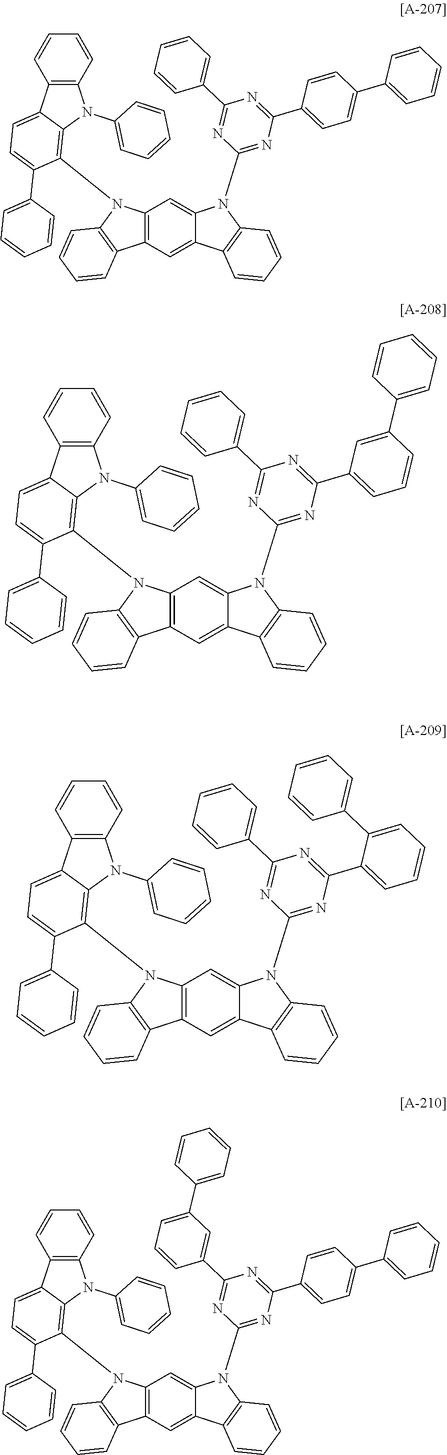 [SMILES: c1ccc(-c2ccc(-c3nc(-c4ccccc4)nc(-n4c5ccccc5c5cc6c7ccccc7n(-c7c(-c8ccccc8)ccc8c9ccccc9n(-c9ccccc9)c78)c6cc54)n3)cc2)cc1 ; c1ccc(-c2cccc(-c3nc(-c4ccccc4)nc(-n4c5ccccc5c5cc6c7ccccc7n(-c7c(-c8ccccc8)ccc8c9ccccc9n(-c9ccccc9)c78)c6cc54)n3)c2)cc1 ; c1ccc(-c2nc(-c3ccccc3-c3ccccc3)nc(-n3c4ccccc4c4cc5c6ccccc6n(-c6c(-c7ccccc7)ccc7c8ccccc8n(-c8ccccc8)c67)c5cc43)n2)cc1 ; c1ccc(-c2ccc(-c3nc(-c4cccc(-c5ccccc5)c4)nc(-n4c5ccccc5c5cc6c7ccccc7n(-c7c(-c8ccccc8)ccc8c9ccccc9n(-c9ccccc9)c78)c6cc54)n3)cc2)cc1]

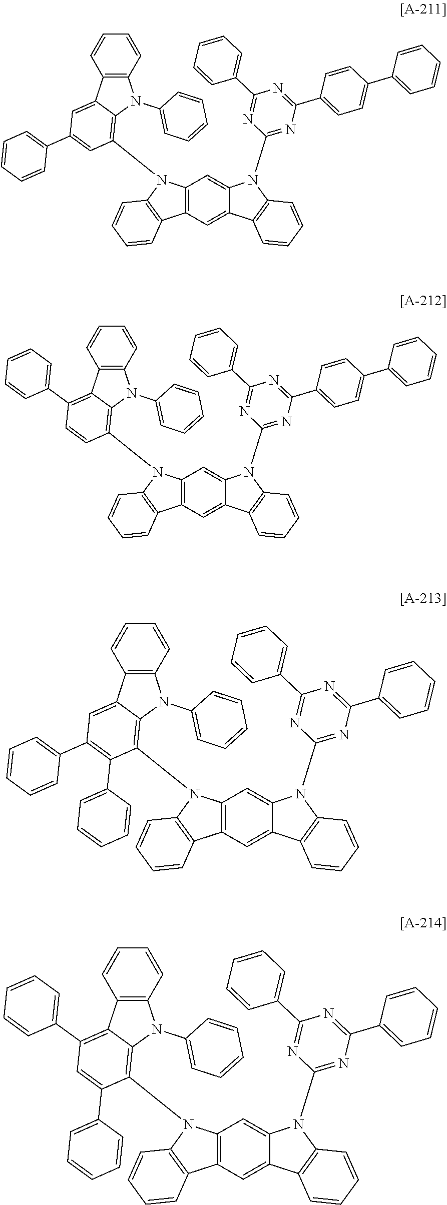 [SMILES: c1ccc(-c2ccc(-c3nc(-c4ccccc4)nc(-n4c5ccccc5c5cc6c7ccccc7n(-c7cc(-c8ccccc8)cc8c9ccccc9n(-c9ccccc9)c78)c6cc54)n3)cc2)cc1 ; c1ccc(-c2ccc(-c3nc(-c4ccccc4)nc(-n4c5ccccc5c5cc6c7ccccc7n(-c7ccc(-c8ccccc8)c8c9ccccc9n(-c9ccccc9)c78)c6cc54)n3)cc2)cc1 ; c1ccc(-c2nc(-c3ccccc3)nc(-n3c4ccccc4c4cc5c6ccccc6n(-c6c(-c7ccccc7)c(-c7ccccc7)cc7c8ccccc8n(-c8ccccc8)c67)c5cc43)n2)cc1 ; c1ccc(-c2nc(-c3ccccc3)nc(-n3c4ccccc4c4cc5c6ccccc6n(-c6c(-c7ccccc7)cc(-c7ccccc7)c7c8ccccc8n(-c8ccccc8)c67)c5cc43)n2)cc1]

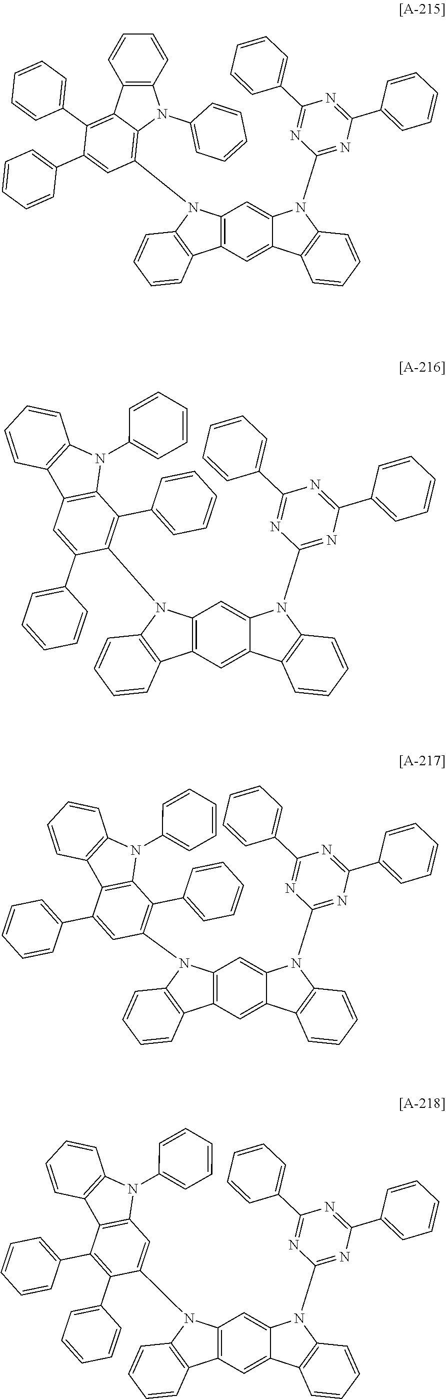 [SMILES: c1ccc(-c2nc(-c3ccccc3)nc(-n3c4ccccc4c4cc5c6ccccc6n(-c6cc(-c7ccccc7)c(-c7ccccc7)c7c8ccccc8n(-c8ccccc8)c67)c5cc43)n2)cc1 ; c1ccc(-c2nc(-c3ccccc3)nc(-n3c4ccccc4c4cc5c6ccccc6n(-c6c(-c7ccccc7)cc7c8ccccc8n(-c8ccccc8)c7c6-c6ccccc6)c5cc43)n2)cc1 ; c1ccc(-c2nc(-c3ccccc3)nc(-n3c4ccccc4c4cc5c6ccccc6n(-c6cc(-c7ccccc7)c7c8ccccc8n(-c8ccccc8)c7c6-c6ccccc6)c5cc43)n2)cc1 ; c1ccc(-c2nc(-c3ccccc3)nc(-n3c4ccccc4c4cc5c6ccccc6n(-c6cc7c(c(-c8ccccc8)c6-c6ccccc6)c6ccccc6n7-c6ccccc6)c5cc43)n2)cc1]

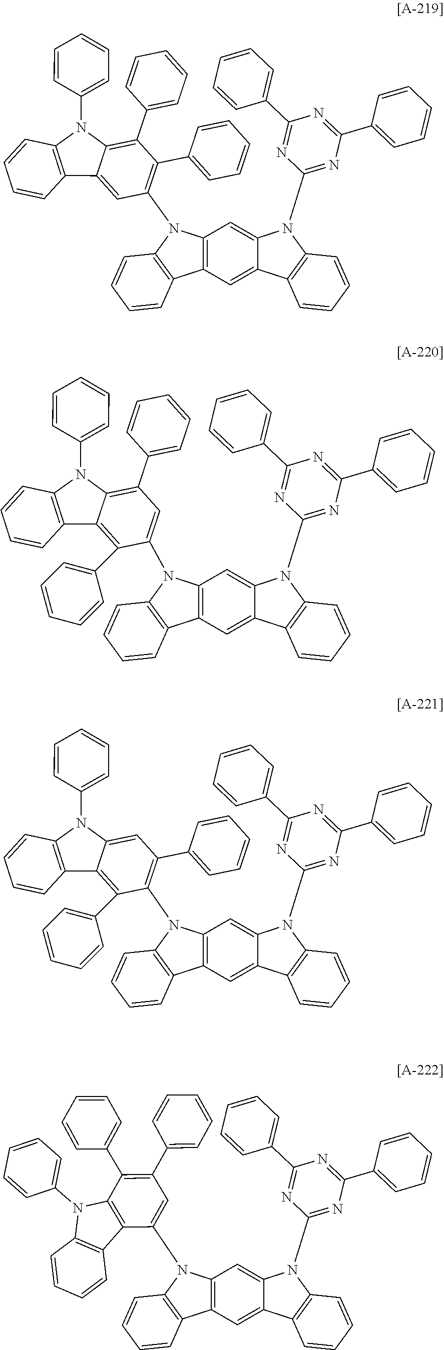 [SMILES: c1ccc(-c2nc(-c3ccccc3)nc(-n3c4ccccc4c4cc5c6ccccc6n(-c6cc7c8ccccc8n(-c8ccccc8)c7c(-c7ccccc7)c6-c6ccccc6)c5cc43)n2)cc1 ; c1ccc(-c2nc(-c3ccccc3)nc(-n3c4ccccc4c4cc5c6ccccc6n(-c6cc(-c7ccccc7)c7c(c6-c6ccccc6)c6ccccc6n7-c6ccccc6)c5cc43)n2)cc1 ; c1ccc(-c2nc(-c3ccccc3)nc(-n3c4ccccc4c4cc5c6ccccc6n(-c6c(-c7ccccc7)cc7c(c6-c6ccccc6)c6ccccc6n7-c6ccccc6)c5cc43)n2)cc1 ; c1ccc(-c2nc(-c3ccccc3)nc(-n3c4ccccc4c4cc5c6ccccc6n(-c6cc(-c7ccccc7)c(-c7ccccc7)c7c6c6ccccc6n7-c6ccccc6)c5cc43)n2)cc1]

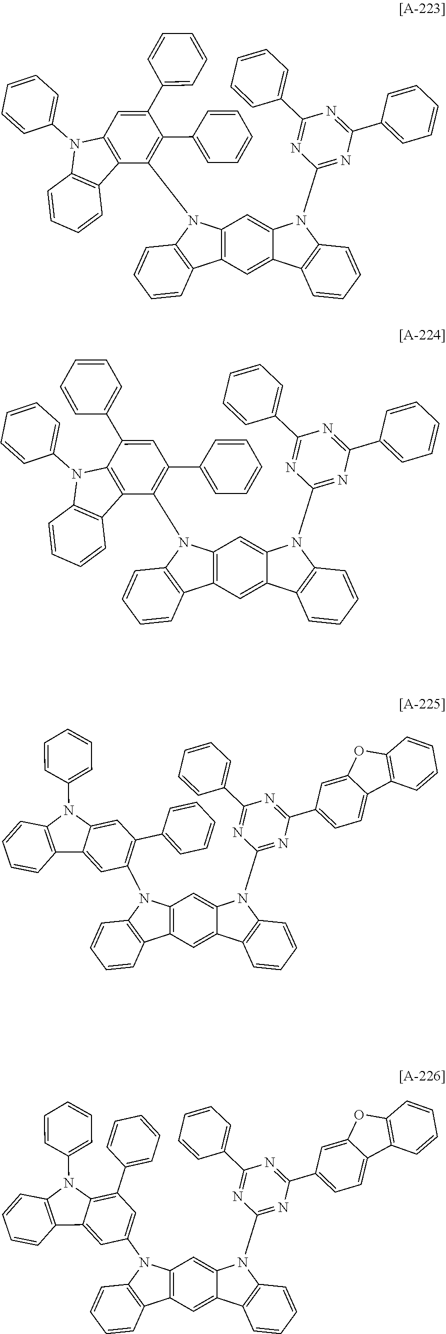 [SMILES: c1ccc(-c2nc(-c3ccccc3)nc(-n3c4ccccc4c4cc5c6ccccc6n(-c6c(-c7ccccc7)c(-c7ccccc7)cc7c6c6ccccc6n7-c6ccccc6)c5cc43)n2)cc1 ; c1ccc(-c2nc(-c3ccccc3)nc(-n3c4ccccc4c4cc5c6ccccc6n(-c6c(-c7ccccc7)cc(-c7ccccc7)c7c6c6ccccc6n7-c6ccccc6)c5cc43)n2)cc1 ; c1ccc(-c2nc(-c3ccc4c(c3)oc3ccccc34)nc(-n3c4ccccc4c4cc5c6ccccc6n(-c6cc7c8ccccc8n(-c8ccccc8)c7cc6-c6ccccc6)c5cc43)n2)cc1 ; c1ccc(-c2nc(-c3ccc4c(c3)oc3ccccc34)nc(-n3c4ccccc4c4cc5c6ccccc6n(-c6cc(-c7ccccc7)c7c(c6)c6ccccc6n7-c6ccccc6)c5cc43)n2)cc1]

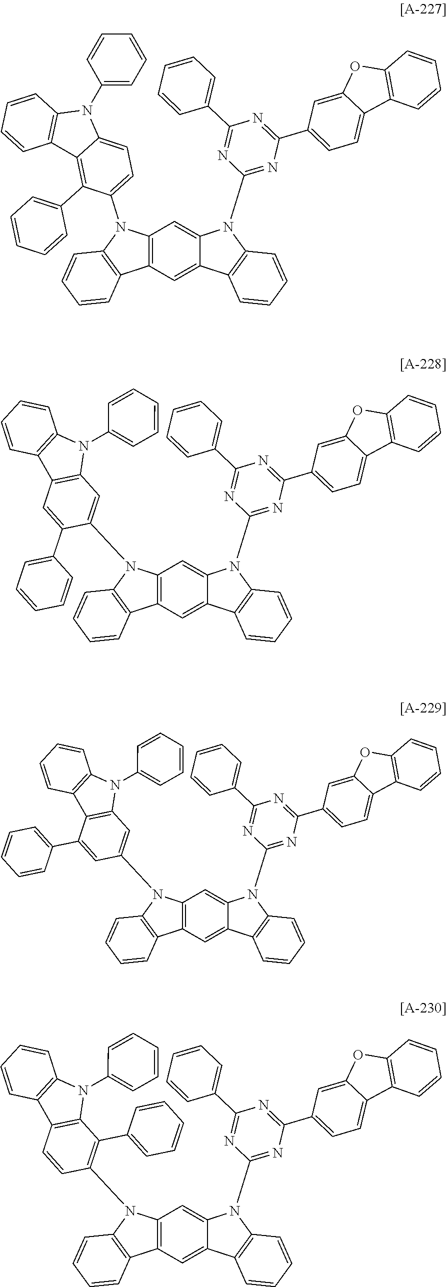 [SMILES: c1ccc(-c2nc(-c3ccc4c(c3)oc3ccccc34)nc(-n3c4ccccc4c4cc5c6ccccc6n(-c6ccc7c(c6-c6ccccc6)c6ccccc6n7-c6ccccc6)c5cc43)n2)cc1 ; c1ccc(-c2nc(-c3ccc4c(c3)oc3ccccc34)nc(-n3c4ccccc4c4cc5c6ccccc6n(-c6cc7c(cc6-c6ccccc6)c6ccccc6n7-c6ccccc6)c5cc43)n2)cc1 ; c1ccc(-c2nc(-c3ccc4c(c3)oc3ccccc34)nc(-n3c4ccccc4c4cc5c6ccccc6n(-c6cc(-c7ccccc7)c7c8ccccc8n(-c8ccccc8)c7c6)c5cc43)n2)cc1 ; c1ccc(-c2nc(-c3ccc4c(c3)oc3ccccc34)nc(-n3c4ccccc4c4cc5c6ccccc6n(-c6ccc7c8ccccc8n(-c8ccccc8)c7c6-c6ccccc6)c5cc43)n2)cc1]

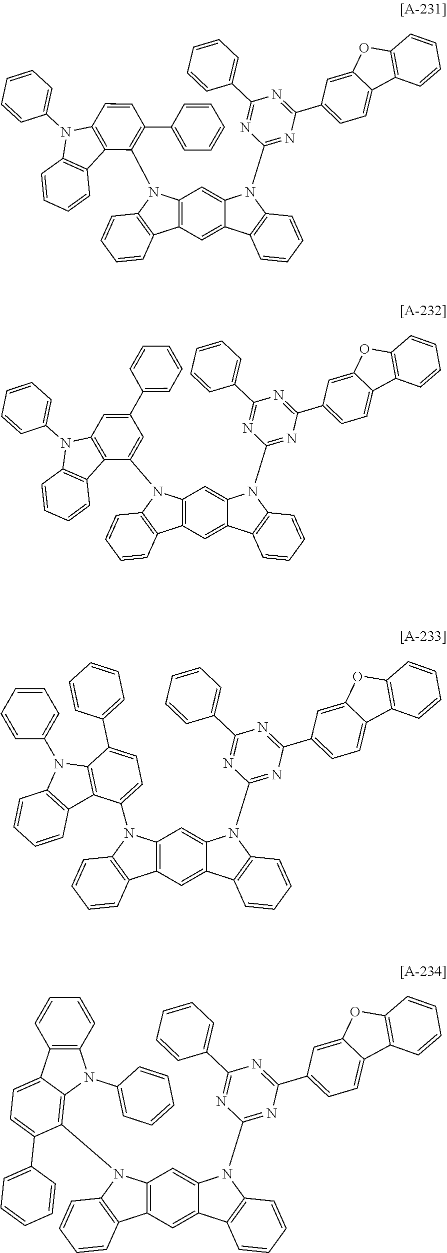 [SMILES: c1ccc(-c2nc(-c3ccc4c(c3)oc3ccccc34)nc(-n3c4ccccc4c4cc5c6ccccc6n(-c6c(-c7ccccc7)ccc7c6c6ccccc6n7-c6ccccc6)c5cc43)n2)cc1 ; c1ccc(-c2cc(-n3c4ccccc4c4cc5c6ccccc6n(-c6nc(-c7ccccc7)nc(-c7ccc8c(c7)oc7ccccc78)n6)c5cc43)c3c4ccccc4n(-c4ccccc4)c3c2)cc1 ; c1ccc(-c2nc(-c3ccc4c(c3)oc3ccccc34)nc(-n3c4ccccc4c4cc5c6ccccc6n(-c6ccc(-c7ccccc7)c7c6c6ccccc6n7-c6ccccc6)c5cc43)n2)cc1 ; c1ccc(-c2nc(-c3ccc4c(c3)oc3ccccc34)nc(-n3c4ccccc4c4cc5c6ccccc6n(-c6c(-c7ccccc7)ccc7c8ccccc8n(-c8ccccc8)c67)c5cc43)n2)cc1]

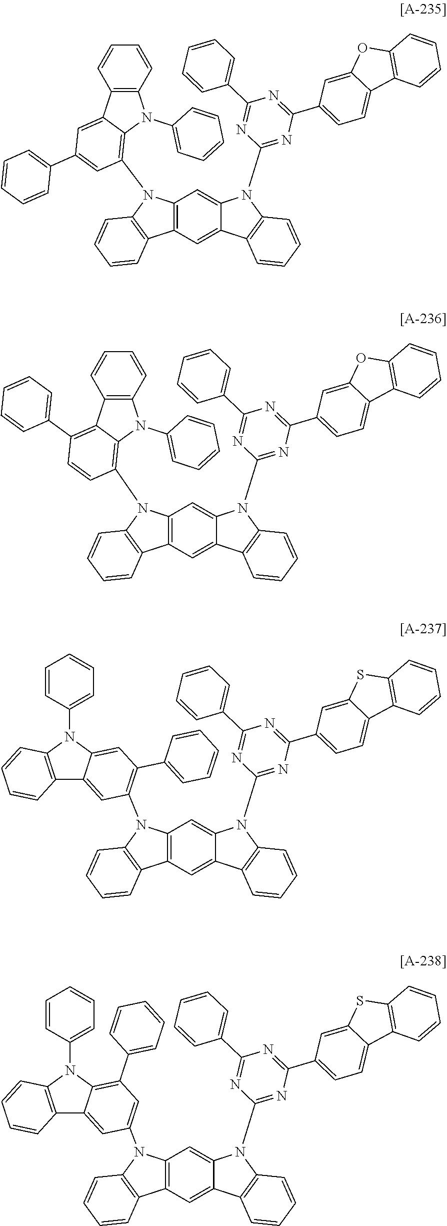 [SMILES: c1ccc(-c2cc(-n3c4ccccc4c4cc5c6ccccc6n(-c6nc(-c7ccccc7)nc(-c7ccc8c(c7)oc7ccccc78)n6)c5cc43)c3c(c2)c2ccccc2n3-c2ccccc2)cc1 ; c1ccc(-c2nc(-c3ccc4c(c3)oc3ccccc34)nc(-n3c4ccccc4c4cc5c6ccccc6n(-c6ccc(-c7ccccc7)c7c8ccccc8n(-c8ccccc8)c67)c5cc43)n2)cc1 ; c1ccc(-c2nc(-c3ccc4c(c3)sc3ccccc34)nc(-n3c4ccccc4c4cc5c6ccccc6n(-c6cc7c8ccccc8n(-c8ccccc8)c7cc6-c6ccccc6)c5cc43)n2)cc1 ; c1ccc(-c2nc(-c3ccc4c(c3)sc3ccccc34)nc(-n3c4ccccc4c4cc5c6ccccc6n(-c6cc(-c7ccccc7)c7c(c6)c6ccccc6n7-c6ccccc6)c5cc43)n2)cc1]

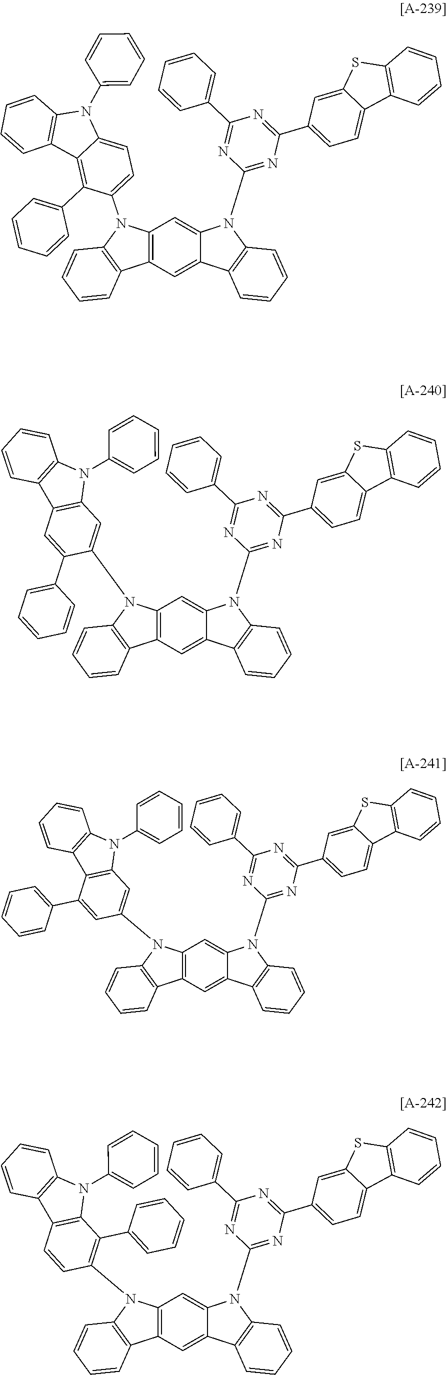 [SMILES: c1ccc(-c2nc(-c3ccc4c(c3)sc3ccccc34)nc(-n3c4ccccc4c4cc5c6ccccc6n(-c6ccc7c(c6-c6ccccc6)c6ccccc6n7-c6ccccc6)c5cc43)n2)cc1 ; c1ccc(-c2nc(-c3ccc4c(c3)sc3ccccc34)nc(-n3c4ccccc4c4cc5c6ccccc6n(-c6cc7c(cc6-c6ccccc6)c6ccccc6n7-c6ccccc6)c5cc43)n2)cc1 ; c1ccc(-c2nc(-c3ccc4c(c3)sc3ccccc34)nc(-n3c4ccccc4c4cc5c6ccccc6n(-c6cc(-c7ccccc7)c7c8ccccc8n(-c8ccccc8)c7c6)c5cc43)n2)cc1 ; c1ccc(-c2nc(-c3ccc4c(c3)sc3ccccc34)nc(-n3c4ccccc4c4cc5c6ccccc6n(-c6ccc7c8ccccc8n(-c8ccccc8)c7c6-c6ccccc6)c5cc43)n2)cc1]

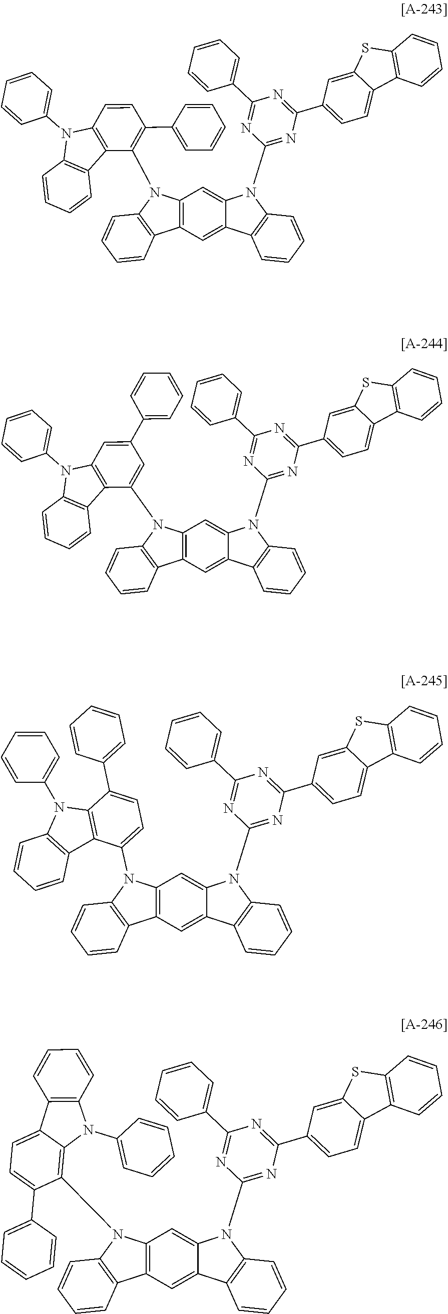 [SMILES: c1ccc(-c2nc(-c3ccc4c(c3)sc3ccccc34)nc(-n3c4ccccc4c4cc5c6ccccc6n(-c6c(-c7ccccc7)ccc7c6c6ccccc6n7-c6ccccc6)c5cc43)n2)cc1 ; c1ccc(-c2cc(-n3c4ccccc4c4cc5c6ccccc6n(-c6nc(-c7ccccc7)nc(-c7ccc8c(c7)sc7ccccc78)n6)c5cc43)c3c4ccccc4n(-c4ccccc4)c3c2)cc1 ; c1ccc(-c2nc(-c3ccc4c(c3)sc3ccccc34)nc(-n3c4ccccc4c4cc5c6ccccc6n(-c6ccc(-c7ccccc7)c7c6c6ccccc6n7-c6ccccc6)c5cc43)n2)cc1 ; c1ccc(-c2nc(-c3ccc4c(c3)sc3ccccc34)nc(-n3c4ccccc4c4cc5c6ccccc6n(-c6c(-c7ccccc7)ccc7c8ccccc8n(-c8ccccc8)c67)c5cc43)n2)cc1]

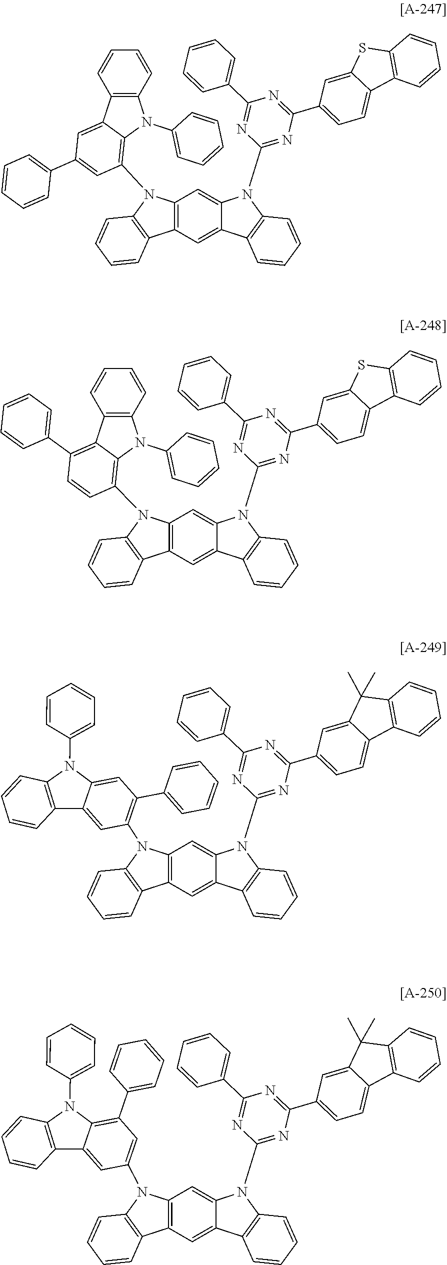 [SMILES: c1ccc(-c2cc(-n3c4ccccc4c4cc5c6ccccc6n(-c6nc(-c7ccccc7)nc(-c7ccc8c(c7)sc7ccccc78)n6)c5cc43)c3c(c2)c2ccccc2n3-c2ccccc2)cc1 ; c1ccc(-c2nc(-c3ccc4c(c3)sc3ccccc34)nc(-n3c4ccccc4c4cc5c6ccccc6n(-c6ccc(-c7ccccc7)c7c8ccccc8n(-c8ccccc8)c67)c5cc43)n2)cc1 ; CC1(C)c2ccccc2-c2ccc(-c3nc(-c4ccccc4)nc(-n4c5ccccc5c5cc6c7ccccc7n(-c7cc8c9ccccc9n(-c9ccccc9)c8cc7-c7ccccc7)c6cc54)n3)cc21 ; CC1(C)c2ccccc2-c2ccc(-c3nc(-c4ccccc4)nc(-n4c5ccccc5c5cc6c7ccccc7n(-c7cc(-c8ccccc8)c8c(c7)c7ccccc7n8-c7ccccc7)c6cc54)n3)cc21]

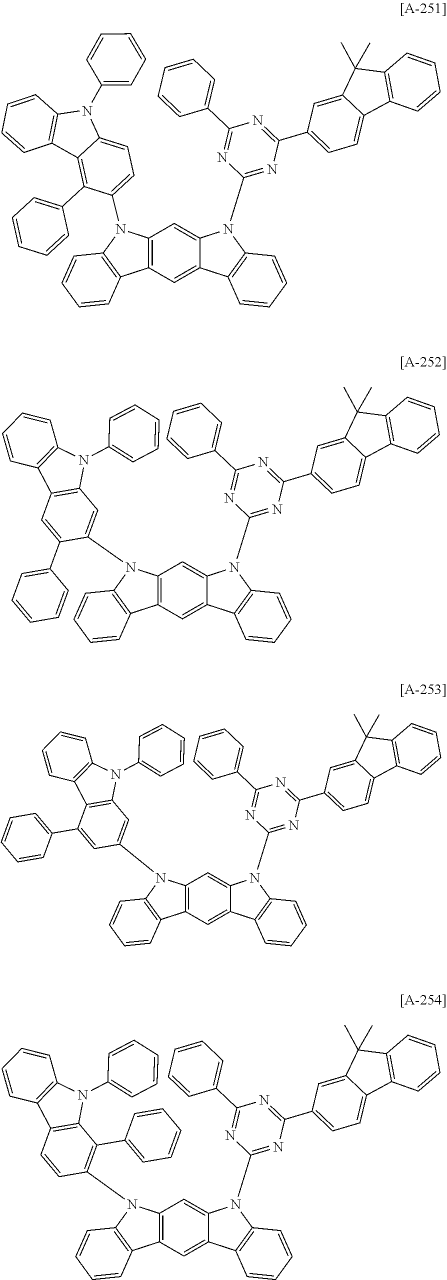 [SMILES: CC1(C)c2ccccc2-c2ccc(-c3nc(-c4ccccc4)nc(-n4c5ccccc5c5cc6c7ccccc7n(-c7ccc8c(c7-c7ccccc7)c7ccccc7n8-c7ccccc7)c6cc54)n3)cc21 ; CC1(C)c2ccccc2-c2ccc(-c3nc(-c4ccccc4)nc(-n4c5ccccc5c5cc6c7ccccc7n(-c7cc8c(cc7-c7ccccc7)c7ccccc7n8-c7ccccc7)c6cc54)n3)cc21 ; CC1(C)c2ccccc2-c2ccc(-c3nc(-c4ccccc4)nc(-n4c5ccccc5c5cc6c7ccccc7n(-c7cc(-c8ccccc8)c8c9ccccc9n(-c9ccccc9)c8c7)c6cc54)n3)cc21 ; CC1(C)c2ccccc2-c2ccc(-c3nc(-c4ccccc4)nc(-n4c5ccccc5c5cc6c7ccccc7n(-c7ccc8c9ccccc9n(-c9ccccc9)c8c7-c7ccccc7)c6cc54)n3)cc21]

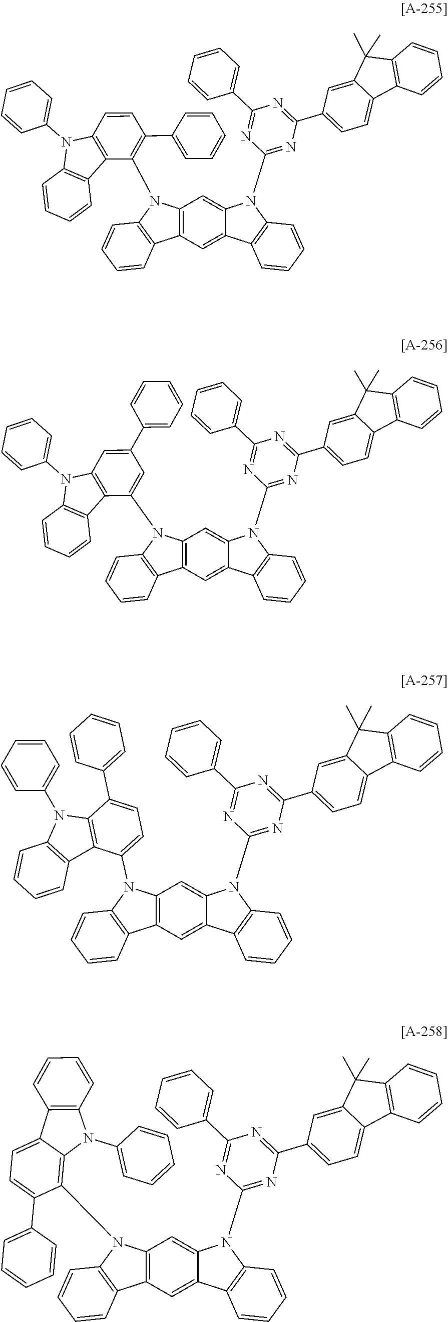 [SMILES: CC1(C)c2ccccc2-c2ccc(-c3nc(-c4ccccc4)nc(-n4c5ccccc5c5cc6c7ccccc7n(-c7c(-c8ccccc8)ccc8c7c7ccccc7n8-c7ccccc7)c6cc54)n3)cc21 ; CC1(C)c2ccccc2-c2ccc(-c3nc(-c4ccccc4)nc(-n4c5ccccc5c5cc6c7ccccc7n(-c7cc(-c8ccccc8)cc8c7c7ccccc7n8-c7ccccc7)c6cc54)n3)cc21 ; CC1(C)c2ccccc2-c2ccc(-c3nc(-c4ccccc4)nc(-n4c5ccccc5c5cc6c7ccccc7n(-c7ccc(-c8ccccc8)c8c7c7ccccc7n8-c7ccccc7)c6cc54)n3)cc21 ; CC1(C)c2ccccc2-c2ccc(-c3nc(-c4ccccc4)nc(-n4c5ccccc5c5cc6c7ccccc7n(-c7c(-c8ccccc8)ccc8c9ccccc9n(-c9ccccc9)c78)c6cc54)n3)cc21]

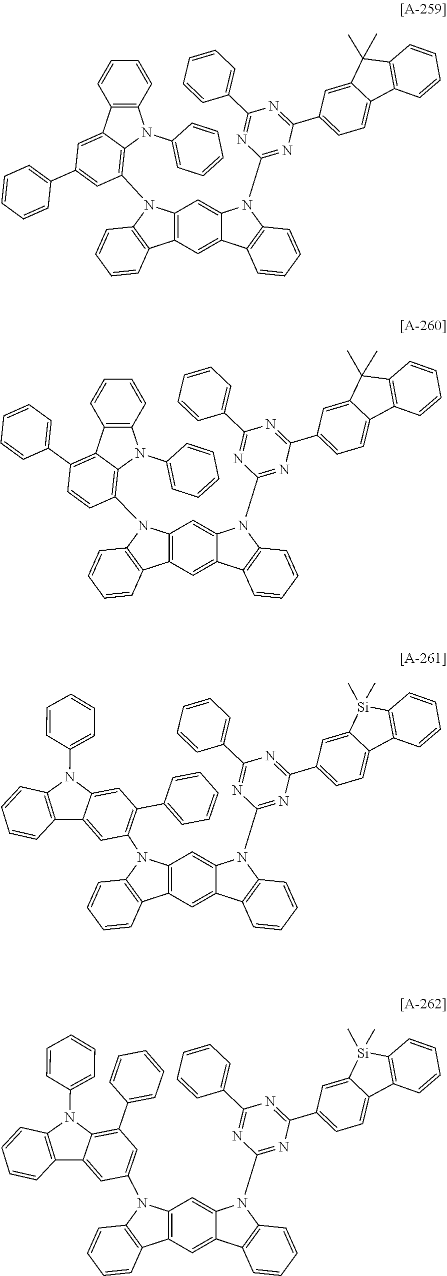

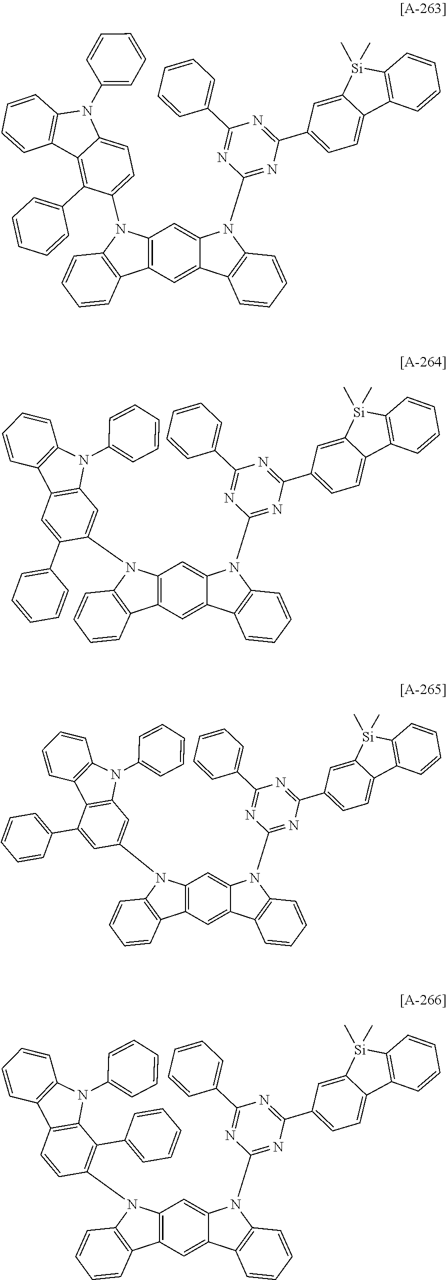

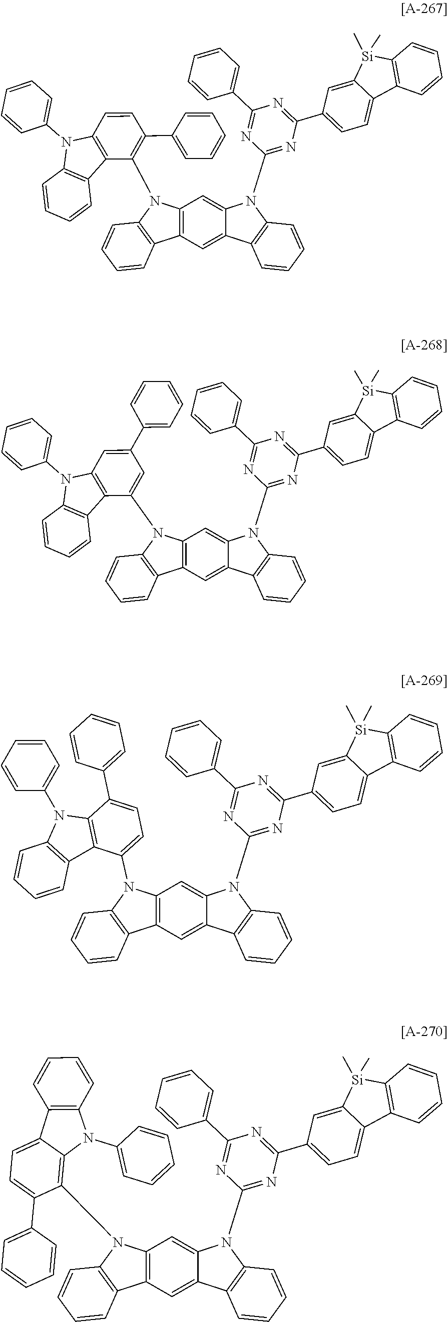

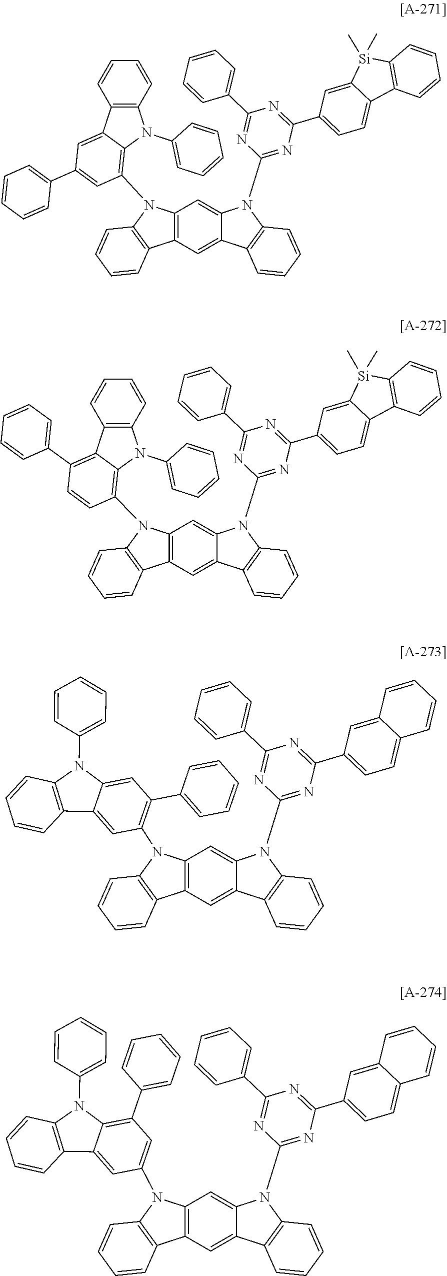 [SMILES: C[Si]1(C)c2ccccc2-c2ccc(-c3nc(-c4ccccc4)nc(-n4c5ccccc5c5cc6c7ccccc7n(-c7cc(-c8ccccc8)cc8c9ccccc9n(-c9ccccc9)c78)c6cc54)n3)cc21 ; C[Si]1(C)c2ccccc2-c2ccc(-c3nc(-c4ccccc4)nc(-n4c5ccccc5c5cc6c7ccccc7n(-c7ccc(-c8ccccc8)c8c9ccccc9n(-c9ccccc9)c78)c6cc54)n3)cc21 ; c1ccc(-c2nc(-c3ccc4ccccc4c3)nc(-n3c4ccccc4c4cc5c6ccccc6n(-c6cc7c8ccccc8n(-c8ccccc8)c7cc6-c6ccccc6)c5cc43)n2)cc1 ; c1ccc(-c2nc(-c3ccc4ccccc4c3)nc(-n3c4ccccc4c4cc5c6ccccc6n(-c6cc(-c7ccccc7)c7c(c6)c6ccccc6n7-c6ccccc6)c5cc43)n2)cc1]

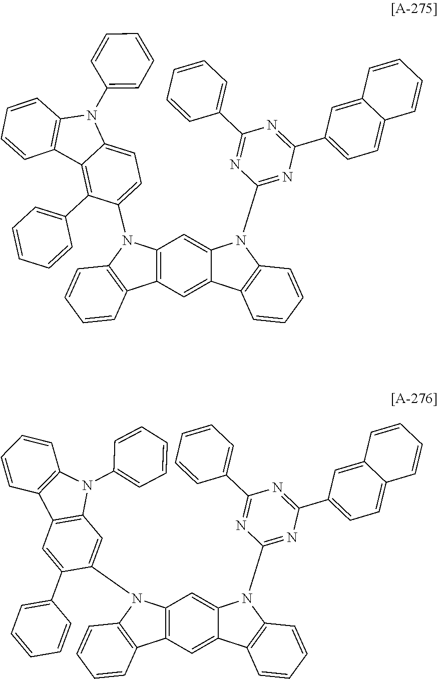 [SMILES: c1ccc(-c2nc(-c3ccc4ccccc4c3)nc(-n3c4ccccc4c4cc5c6ccccc6n(-c6ccc7c(c6-c6ccccc6)c6ccccc6n7-c6ccccc6)c5cc43)n2)cc1 ; c1ccc(-c2nc(-c3ccc4ccccc4c3)nc(-n3c4ccccc4c4cc5c6ccccc6n(-c6cc7c(cc6-c6ccccc6)c6ccccc6n7-c6ccccc6)c5cc43)n2)cc1]

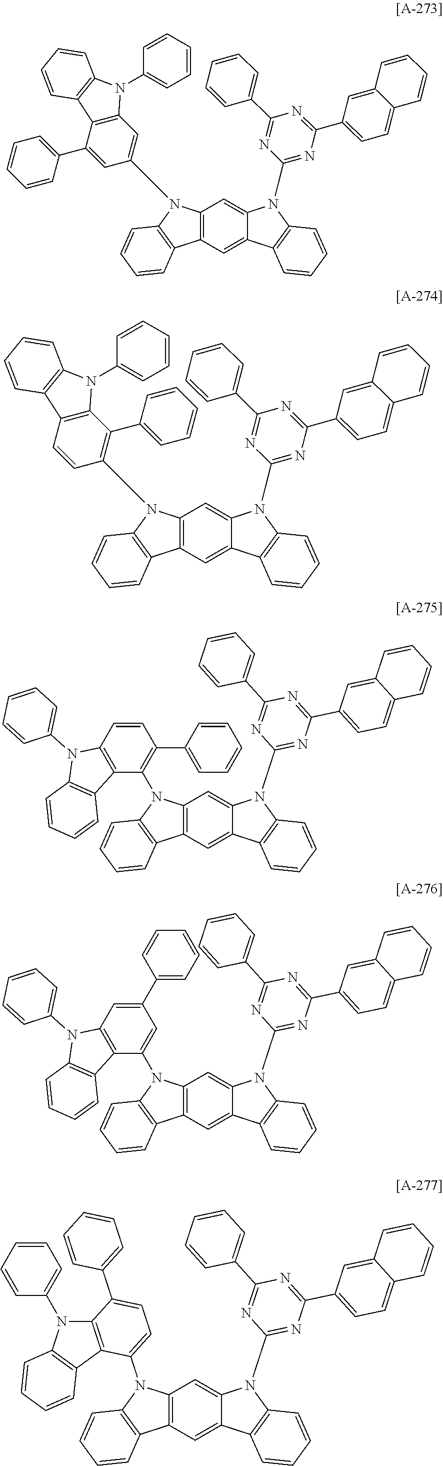

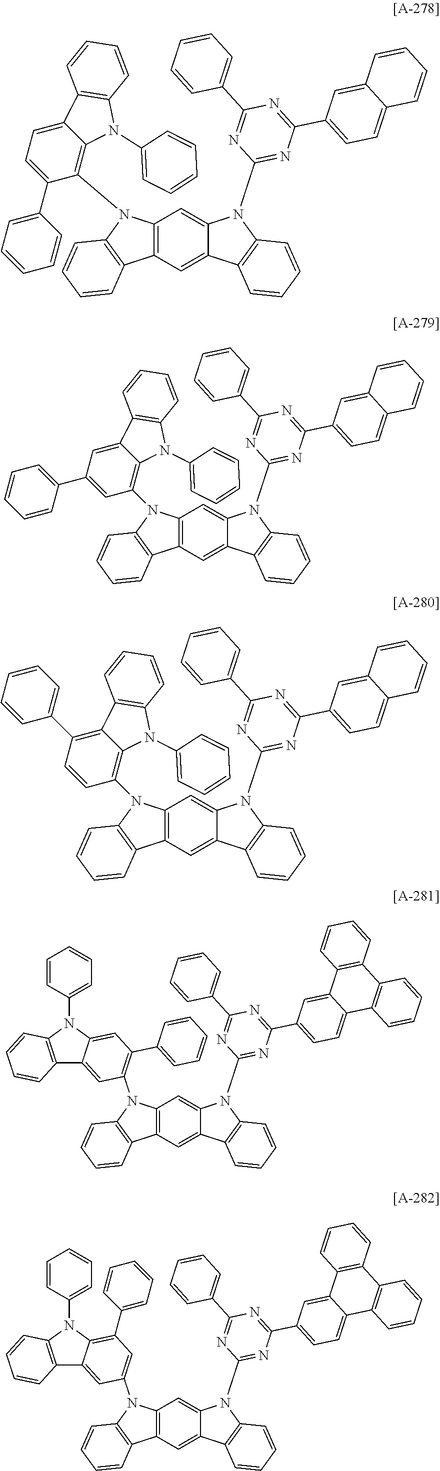

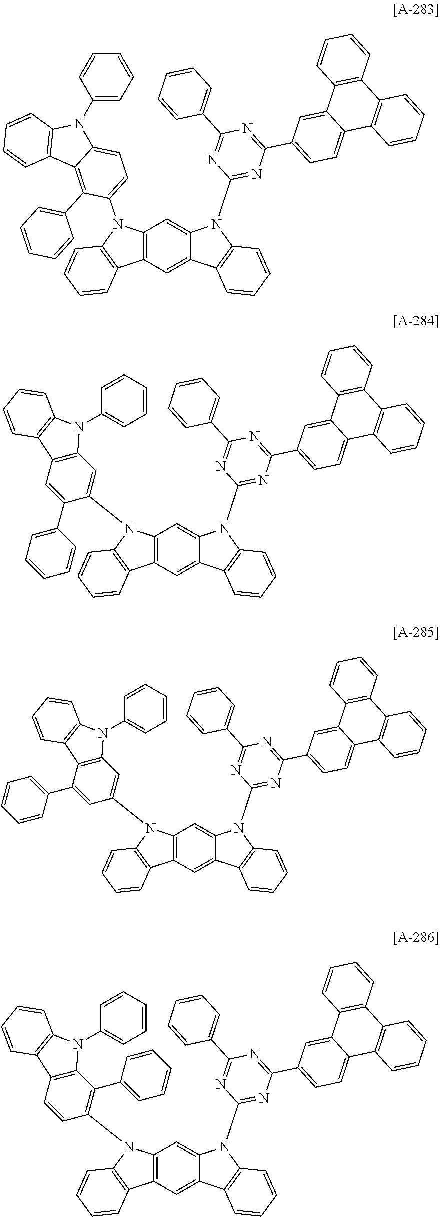

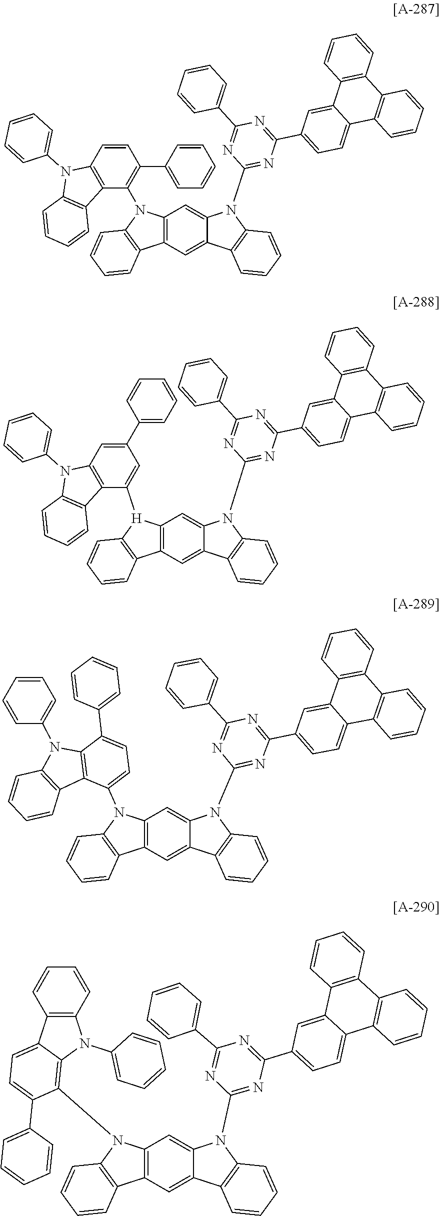

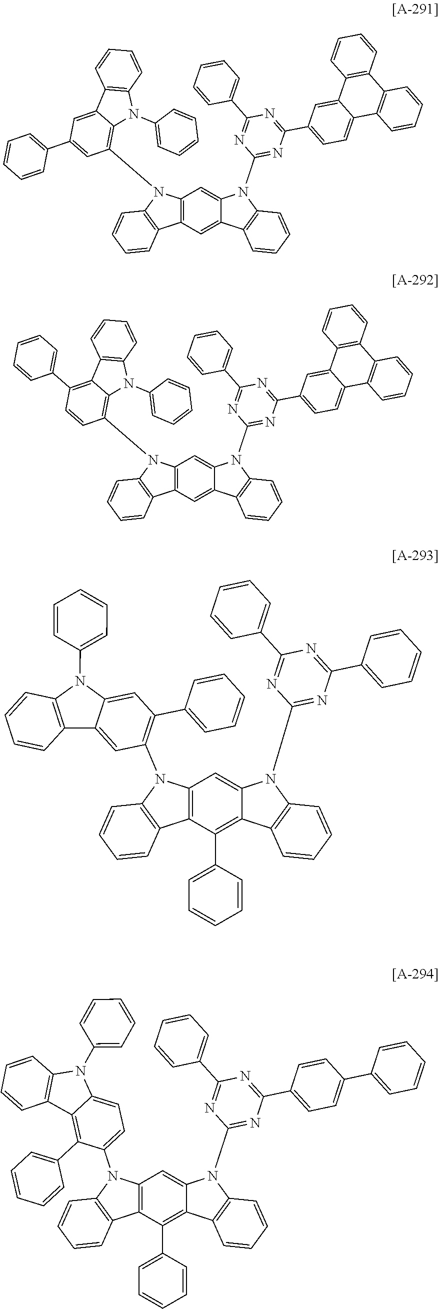

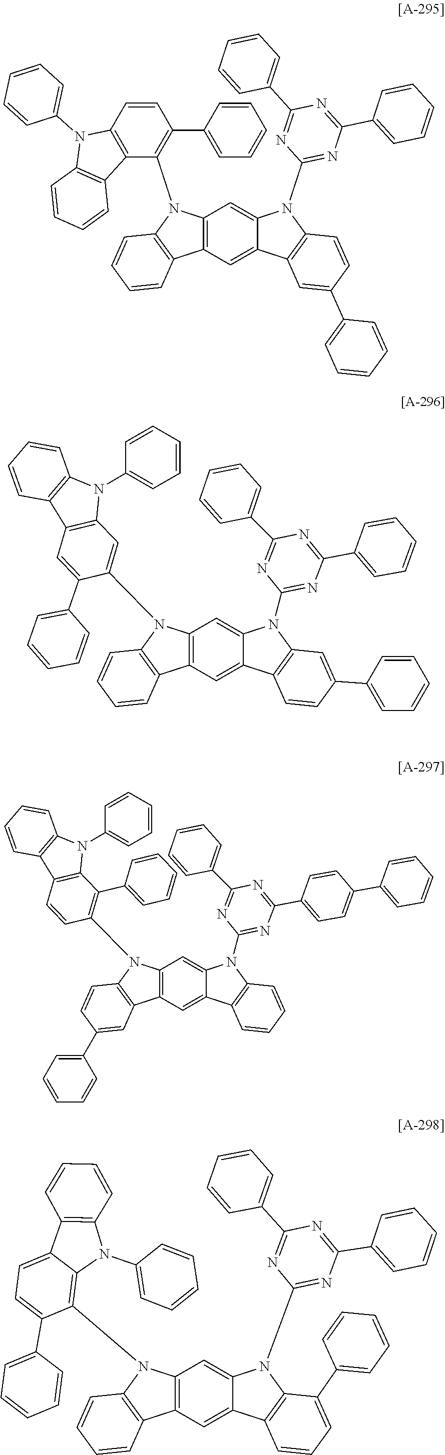 [SMILES: c1ccc(-c2ccc3c(c2)c2cc4c5ccccc5n(-c5c(-c6ccccc6)ccc6c5c5ccccc5n6-c5ccccc5)c4cc2n3-c2nc(-c3ccccc3)nc(-c3ccccc3)n2)cc1 ; c1ccc(-c2ccc3c4cc5c6ccccc6n(-c6cc7c(cc6-c6ccccc6)c6ccccc6n7-c6ccccc6)c5cc4n(-c4nc(-c5ccccc5)nc(-c5ccccc5)n4)c3c2)cc1 ; c1ccc(-c2ccc(-c3nc(-c4ccccc4)nc(-n4c5ccccc5c5cc6c7cc(-c8ccccc8)ccc7n(-c7ccc8c9ccccc9n(-c9ccccc9)c8c7-c7ccccc7)c6cc54)n3)cc2)cc1 ; c1ccc(-c2nc(-c3ccccc3)nc(-n3c4cc5c(cc4c4cccc(-c6ccccc6)c43)c3ccccc3n5-c3c(-c4ccccc4)ccc4c5ccccc5n(-c5ccccc5)c34)n2)cc1]

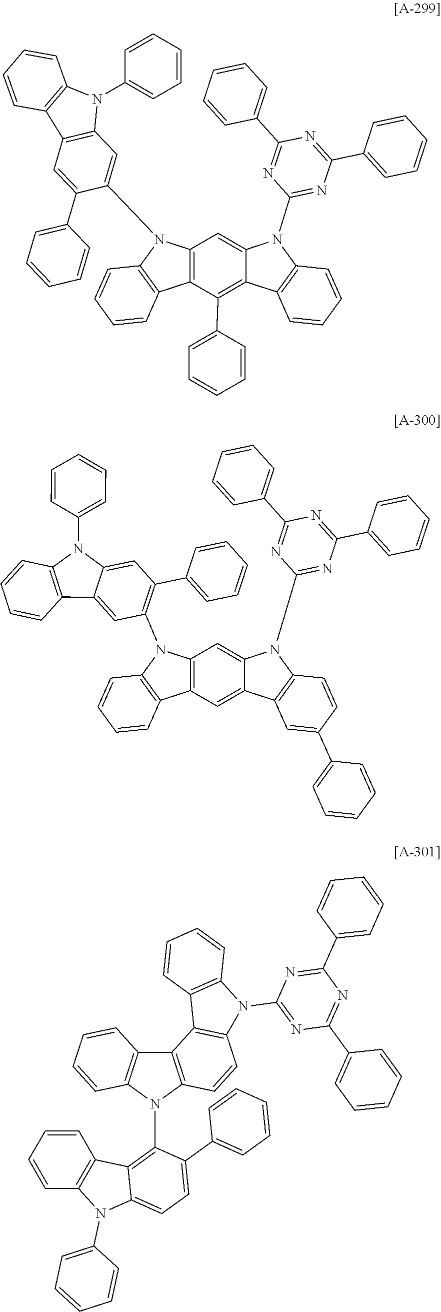 [SMILES: c1ccc(-c2nc(-c3ccccc3)nc(-n3c4ccccc4c4c(-c5ccccc5)c5c6ccccc6n(-c6cc7c(cc6-c6ccccc6)c6ccccc6n7-c6ccccc6)c5cc43)n2)cc1 ; c1ccc(-c2ccc3c(c2)c2cc4c5ccccc5n(-c5cc6c7ccccc7n(-c7ccccc7)c6cc5-c5ccccc5)c4cc2n3-c2nc(-c3ccccc3)nc(-c3ccccc3)n2)cc1 ; c1ccc(-c2nc(-c3ccccc3)nc(-n3c4ccccc4c4c5c6ccccc6n(-c6c(-c7ccccc7)ccc7c6c6ccccc6n7-c6ccccc6)c5ccc43)n2)cc1]

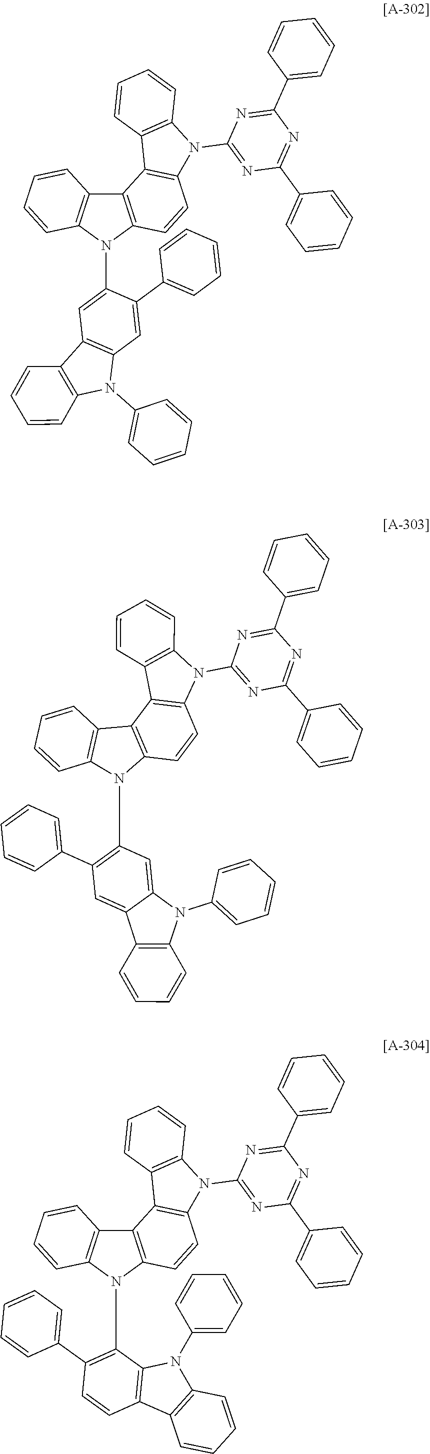 [SMILES: c1ccc(-c2nc(-c3ccccc3)nc(-n3c4ccccc4c4c5c6ccccc6n(-c6cc7c8ccccc8n(-c8ccccc8)c7cc6-c6ccccc6)c5ccc43)n2)cc1 ; c1ccc(-c2nc(-c3ccccc3)nc(-n3c4ccccc4c4c5c6ccccc6n(-c6cc7c(cc6-c6ccccc6)c6ccccc6n7-c6ccccc6)c5ccc43)n2)cc1 ; c1ccc(-c2nc(-c3ccccc3)nc(-n3c4ccccc4c4c5c6ccccc6n(-c6c(-c7ccccc7)ccc7c8ccccc8n(-c8ccccc8)c67)c5ccc43)n2)cc1]

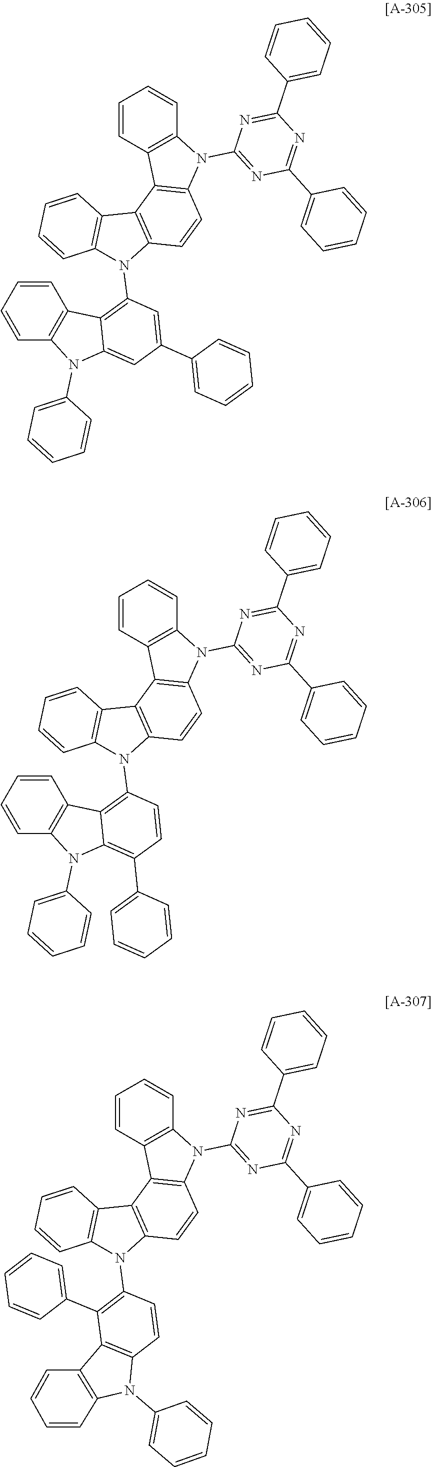 [SMILES: c1ccc(-c2cc(-n3c4ccccc4c4c5c6ccccc6n(-c6nc(-c7ccccc7)nc(-c7ccccc7)n6)c5ccc43)c3c4ccccc4n(-c4ccccc4)c3c2)cc1 ; c1ccc(-c2nc(-c3ccccc3)nc(-n3c4ccccc4c4c5c6ccccc6n(-c6ccc(-c7ccccc7)c7c6c6ccccc6n7-c6ccccc6)c5ccc43)n2)cc1 ; c1ccc(-c2nc(-c3ccccc3)nc(-n3c4ccccc4c4c5c6ccccc6n(-c6ccc7c(c6-c6ccccc6)c6ccccc6n7-c6ccccc6)c5ccc43)n2)cc1]

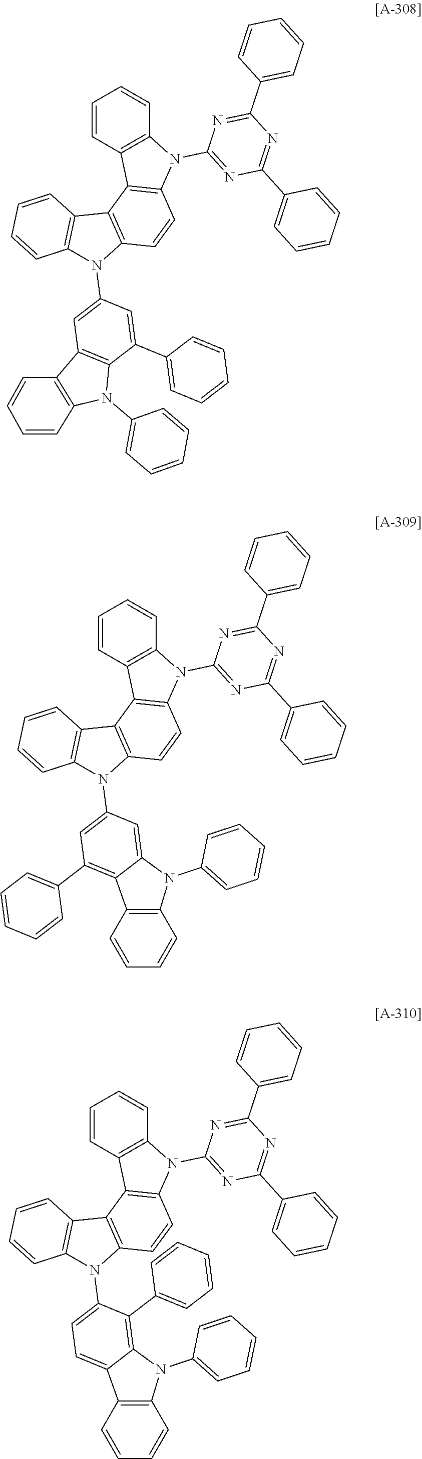 [SMILES: c1ccc(-c2nc(-c3ccccc3)nc(-n3c4ccccc4c4c5c6ccccc6n(-c6cc(-c7ccccc7)c7c(c6)c6ccccc6n7-c6ccccc6)c5ccc43)n2)cc1 ; c1ccc(-c2nc(-c3ccccc3)nc(-n3c4ccccc4c4c5c6ccccc6n(-c6cc(-c7ccccc7)c7c8ccccc8n(-c8ccccc8)c7c6)c5ccc43)n2)cc1 ; c1ccc(-c2nc(-c3ccccc3)nc(-n3c4ccccc4c4c5c6ccccc6n(-c6ccc7c8ccccc8n(-c8ccccc8)c7c6-c6ccccc6)c5ccc43)n2)cc1]

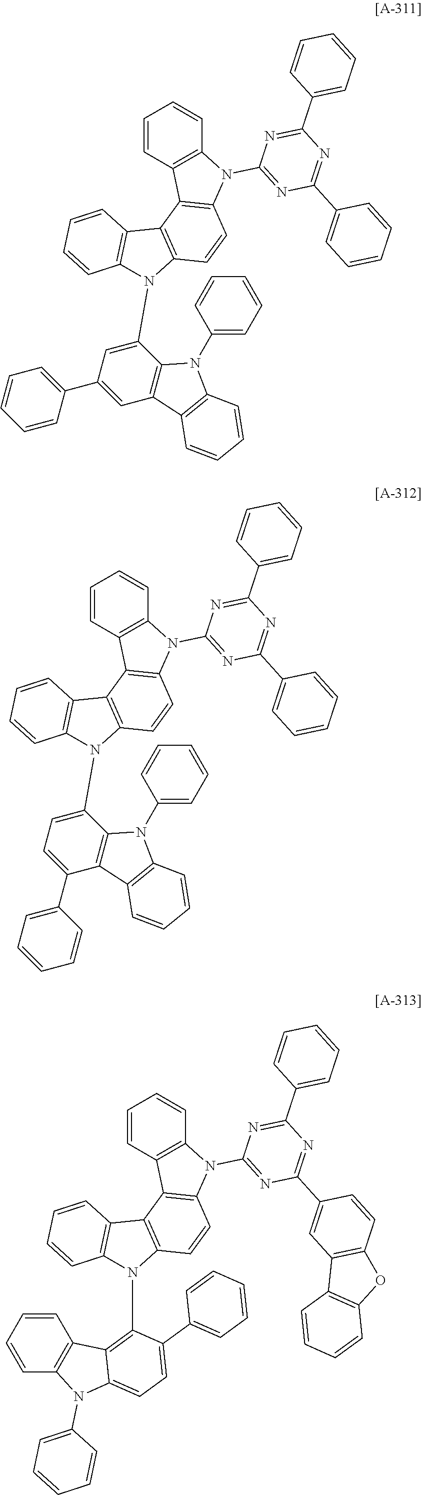 [SMILES: c1ccc(-c2cc(-n3c4ccccc4c4c5c6ccccc6n(-c6nc(-c7ccccc7)nc(-c7ccccc7)n6)c5ccc43)c3c(c2)c2ccccc2n3-c2ccccc2)cc1 ; c1ccc(-c2nc(-c3ccccc3)nc(-n3c4ccccc4c4c5c6ccccc6n(-c6ccc(-c7ccccc7)c7c8ccccc8n(-c8ccccc8)c67)c5ccc43)n2)cc1 ; c1ccc(-c2nc(-c3ccc4oc5ccccc5c4c3)nc(-n3c4ccccc4c4c5c6ccccc6n(-c6c(-c7ccccc7)ccc7c6c6ccccc6n7-c6ccccc6)c5ccc43)n2)cc1]

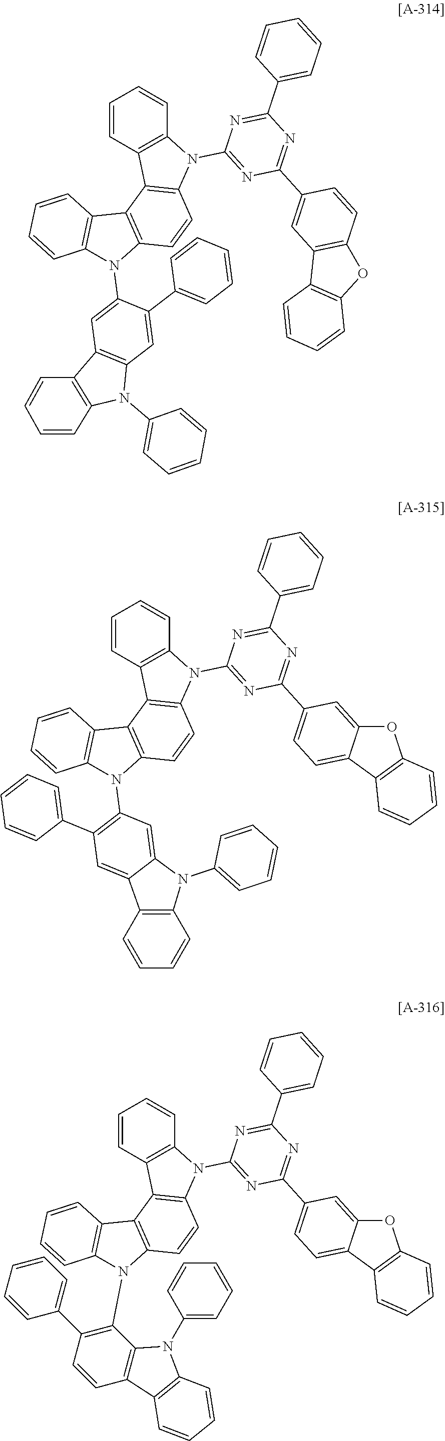 [SMILES: c1ccc(-c2nc(-c3ccc4oc5ccccc5c4c3)nc(-n3c4ccccc4c4c5c6ccccc6n(-c6cc7c8ccccc8n(-c8ccccc8)c7cc6-c6ccccc6)c5ccc43)n2)cc1 ; c1ccc(-c2nc(-c3ccc4c(c3)oc3ccccc34)nc(-n3c4ccccc4c4c5c6ccccc6n(-c6cc7c(cc6-c6ccccc6)c6ccccc6n7-c6ccccc6)c5ccc43)n2)cc1 ; c1ccc(-c2nc(-c3ccc4c(c3)oc3ccccc34)nc(-n3c4ccccc4c4c5c6ccccc6n(-c6c(-c7ccccc7)ccc7c8ccccc8n(-c8ccccc8)c67)c5ccc43)n2)cc1]

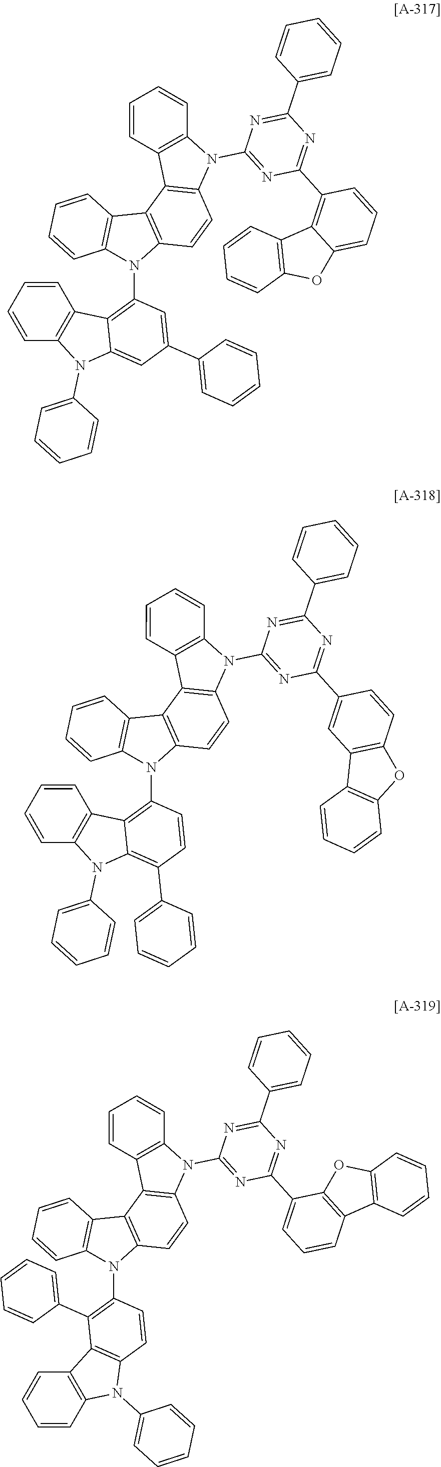 [SMILES: c1ccc(-c2cc(-n3c4ccccc4c4c5c6ccccc6n(-c6nc(-c7ccccc7)nc(-c7cccc8oc9ccccc9c78)n6)c5ccc43)c3c4ccccc4n(-c4ccccc4)c3c2)cc1 ; c1ccc(-c2nc(-c3ccc4oc5ccccc5c4c3)nc(-n3c4ccccc4c4c5c6ccccc6n(-c6ccc(-c7ccccc7)c7c6c6ccccc6n7-c6ccccc6)c5ccc43)n2)cc1 ; c1ccc(-c2nc(-c3cccc4c3oc3ccccc34)nc(-n3c4ccccc4c4c5c6ccccc6n(-c6ccc7c(c6-c6ccccc6)c6ccccc6n7-c6ccccc6)c5ccc43)n2)cc1]

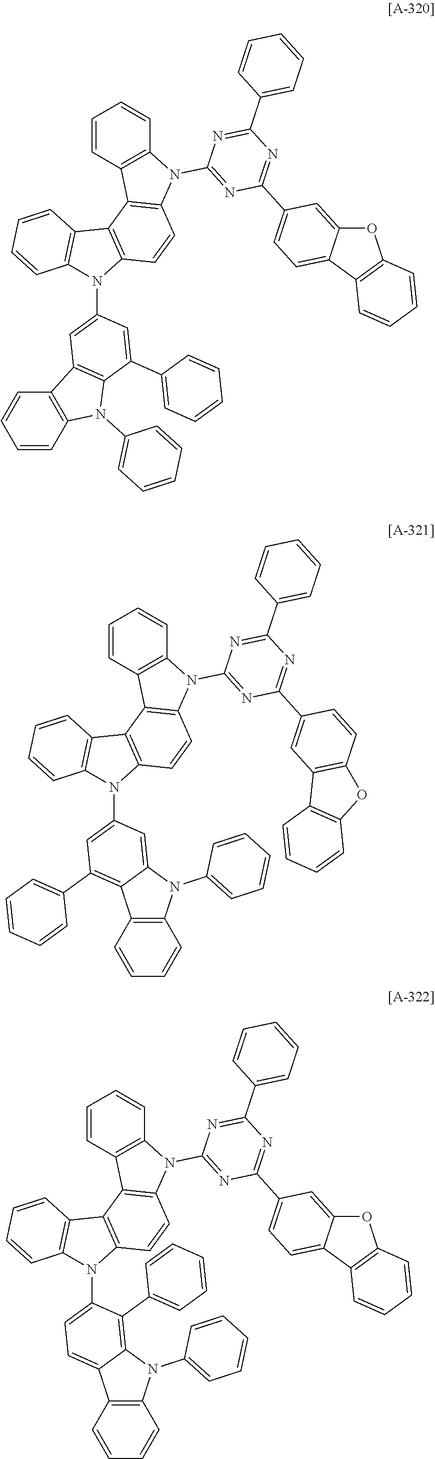 [SMILES: c1ccc(-c2nc(-c3ccc4c(c3)oc3ccccc34)nc(-n3c4ccccc4c4c5c6ccccc6n(-c6cc(-c7ccccc7)c7c(c6)c6ccccc6n7-c6ccccc6)c5ccc43)n2)cc1 ; c1ccc(-c2nc(-c3ccc4oc5ccccc5c4c3)nc(-n3c4ccccc4c4c5c6ccccc6n(-c6cc(-c7ccccc7)c7c8ccccc8n(-c8ccccc8)c7c6)c5ccc43)n2)cc1 ; c1ccc(-c2nc(-c3ccc4c(c3)oc3ccccc34)nc(-n3c4ccccc4c4c5c6ccccc6n(-c6ccc7c8ccccc8n(-c8ccccc8)c7c6-c6ccccc6)c5ccc43)n2)cc1]

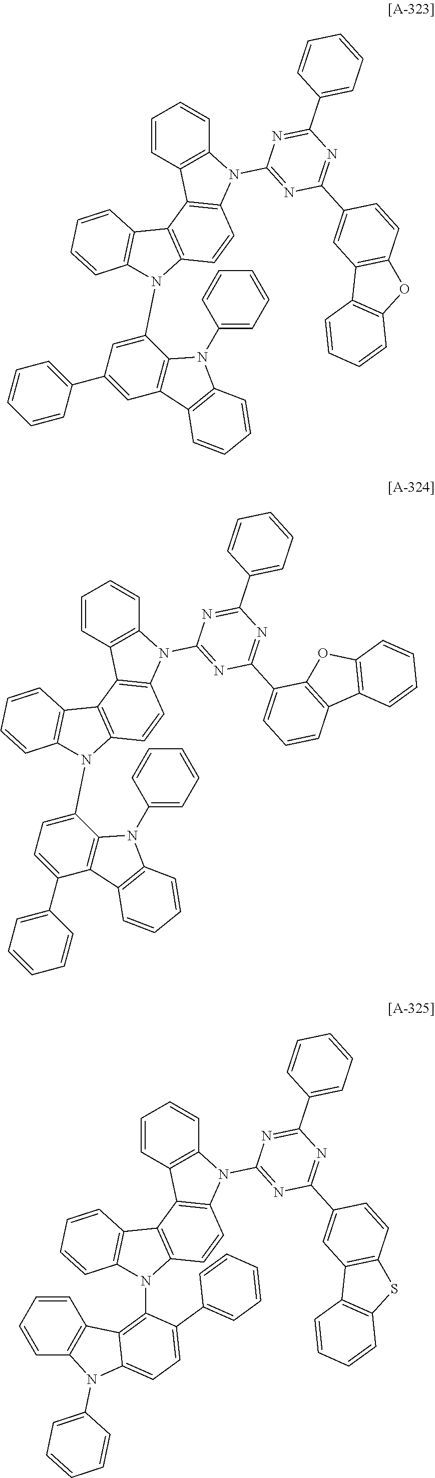 [SMILES: c1ccc(-c2cc(-n3c4ccccc4c4c5c6ccccc6n(-c6nc(-c7ccccc7)nc(-c7ccc8oc9ccccc9c8c7)n6)c5ccc43)c3c(c2)c2ccccc2n3-c2ccccc2)cc1 ; c1ccc(-c2nc(-c3cccc4c3oc3ccccc34)nc(-n3c4ccccc4c4c5c6ccccc6n(-c6ccc(-c7ccccc7)c7c8ccccc8n(-c8ccccc8)c67)c5ccc43)n2)cc1 ; c1ccc(-c2nc(-c3ccc4sc5ccccc5c4c3)nc(-n3c4ccccc4c4c5c6ccccc6n(-c6c(-c7ccccc7)ccc7c6c6ccccc6n7-c6ccccc6)c5ccc43)n2)cc1]

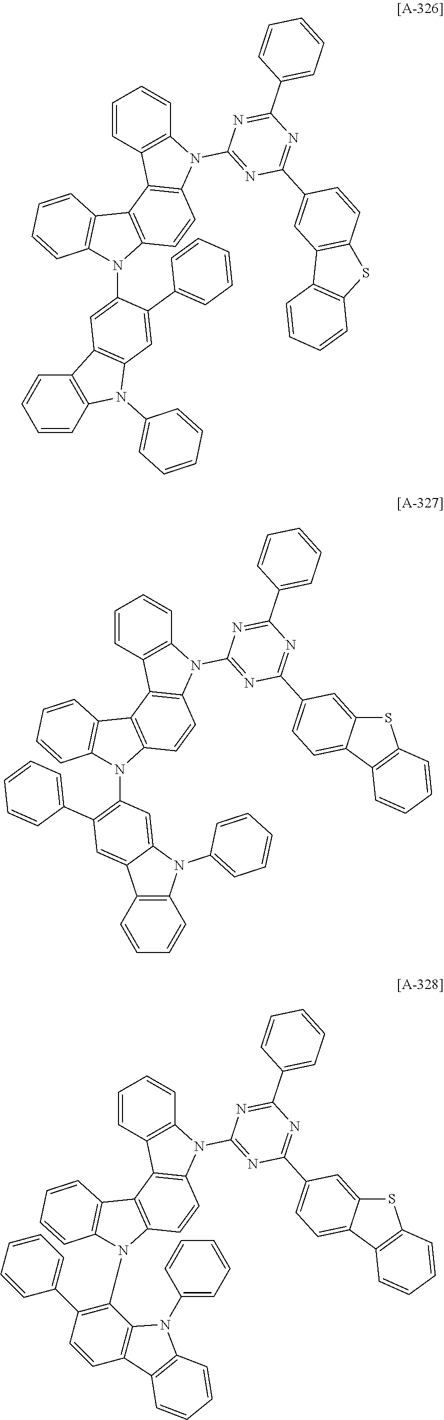 [SMILES: c1ccc(-c2nc(-c3ccc4sc5ccccc5c4c3)nc(-n3c4ccccc4c4c5c6ccccc6n(-c6cc7c8ccccc8n(-c8ccccc8)c7cc6-c6ccccc6)c5ccc43)n2)cc1 ; c1ccc(-c2nc(-c3ccc4c(c3)sc3ccccc34)nc(-n3c4ccccc4c4c5c6ccccc6n(-c6cc7c(cc6-c6ccccc6)c6ccccc6n7-c6ccccc6)c5ccc43)n2)cc1 ; c1ccc(-c2nc(-c3ccc4c(c3)sc3ccccc34)nc(-n3c4ccccc4c4c5c6ccccc6n(-c6c(-c7ccccc7)ccc7c8ccccc8n(-c8ccccc8)c67)c5ccc43)n2)cc1]

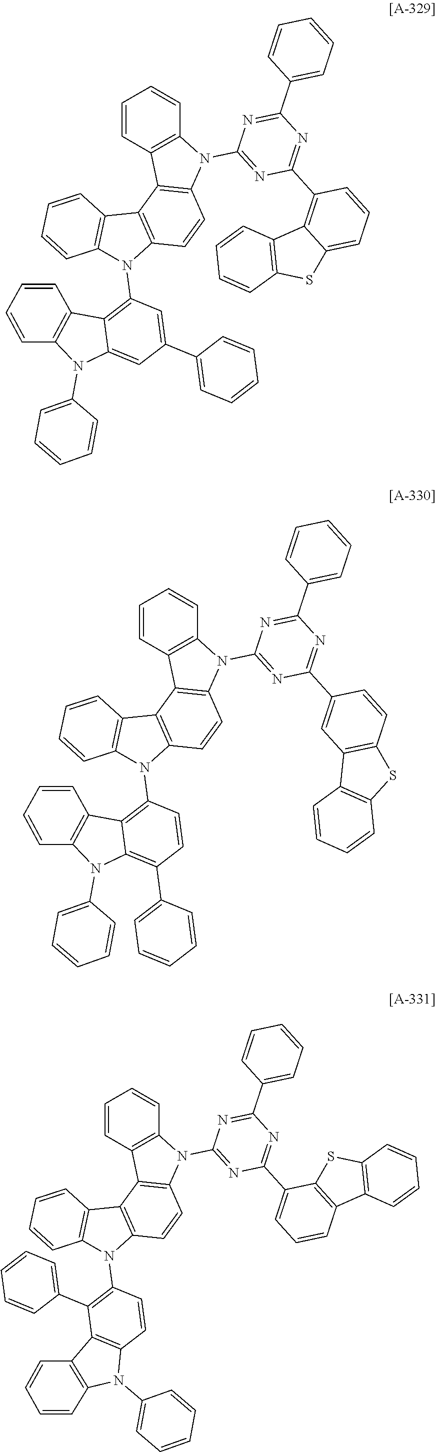 [SMILES: c1ccc(-c2cc(-n3c4ccccc4c4c5c6ccccc6n(-c6nc(-c7ccccc7)nc(-c7cccc8sc9ccccc9c78)n6)c5ccc43)c3c4ccccc4n(-c4ccccc4)c3c2)cc1 ; c1ccc(-c2nc(-c3ccc4sc5ccccc5c4c3)nc(-n3c4ccccc4c4c5c6ccccc6n(-c6ccc(-c7ccccc7)c7c6c6ccccc6n7-c6ccccc6)c5ccc43)n2)cc1 ; c1ccc(-c2nc(-c3cccc4c3sc3ccccc34)nc(-n3c4ccccc4c4c5c6ccccc6n(-c6ccc7c(c6-c6ccccc6)c6ccccc6n7-c6ccccc6)c5ccc43)n2)cc1]

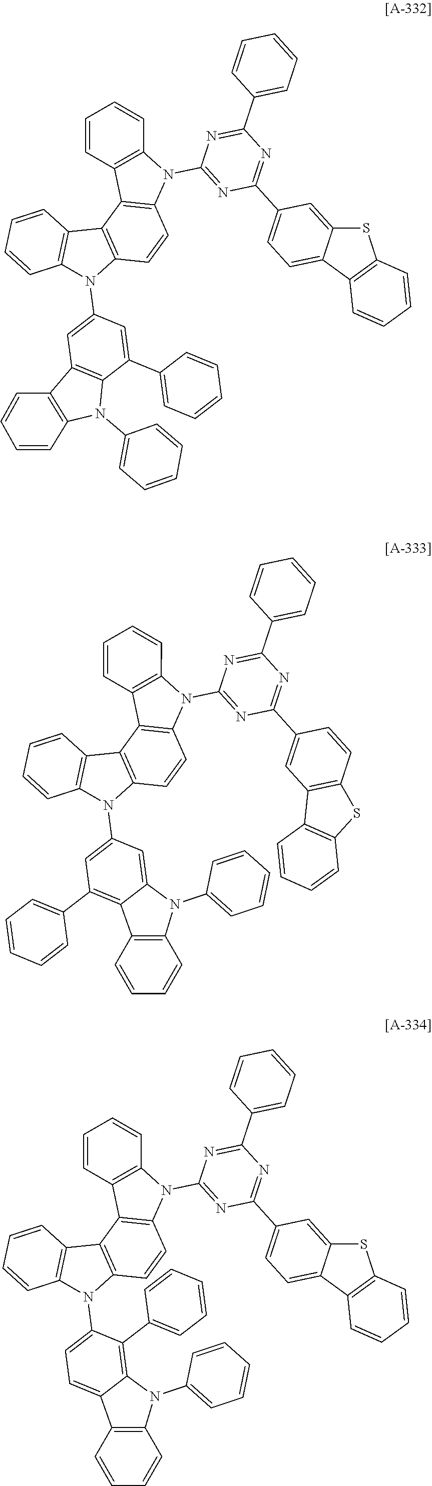 [SMILES: c1ccc(-c2nc(-c3ccc4c(c3)sc3ccccc34)nc(-n3c4ccccc4c4c5c6ccccc6n(-c6cc(-c7ccccc7)c7c(c6)c6ccccc6n7-c6ccccc6)c5ccc43)n2)cc1 ; c1ccc(-c2nc(-c3ccc4sc5ccccc5c4c3)nc(-n3c4ccccc4c4c5c6ccccc6n(-c6cc(-c7ccccc7)c7c8ccccc8n(-c8ccccc8)c7c6)c5ccc43)n2)cc1 ; c1ccc(-c2nc(-c3ccc4c(c3)sc3ccccc34)nc(-n3c4ccccc4c4c5c6ccccc6n(-c6ccc7c8ccccc8n(-c8ccccc8)c7c6-c6ccccc6)c5ccc43)n2)cc1]

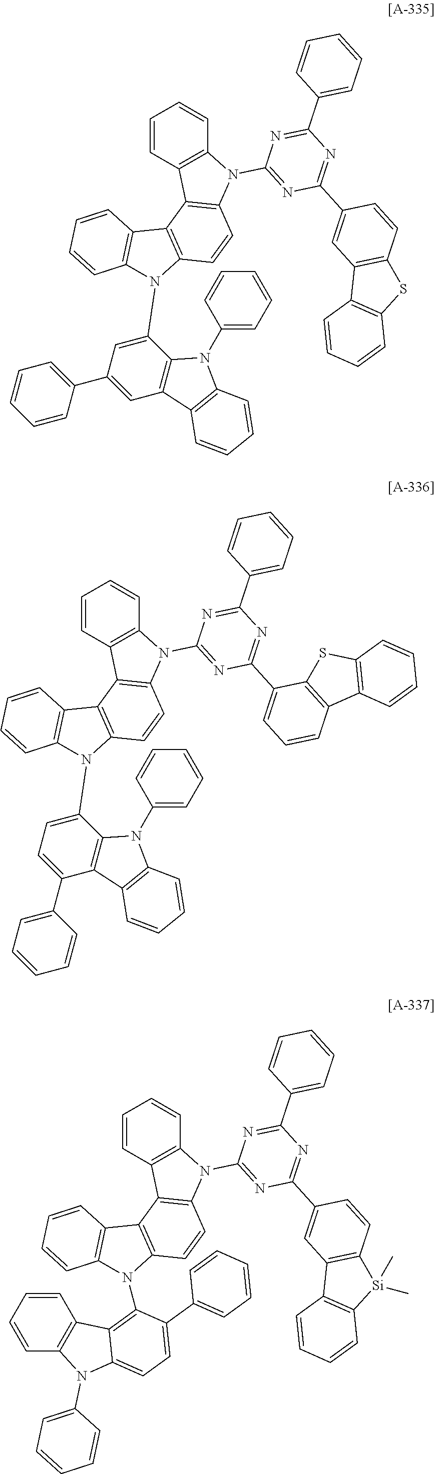 [SMILES: c1ccc(-c2cc(-n3c4ccccc4c4c5c6ccccc6n(-c6nc(-c7ccccc7)nc(-c7ccc8sc9ccccc9c8c7)n6)c5ccc43)c3c(c2)c2ccccc2n3-c2ccccc2)cc1 ; c1ccc(-c2nc(-c3cccc4c3sc3ccccc34)nc(-n3c4ccccc4c4c5c6ccccc6n(-c6ccc(-c7ccccc7)c7c8ccccc8n(-c8ccccc8)c67)c5ccc43)n2)cc1 ; C[Si]1(C)c2ccccc2-c2cc(-c3nc(-c4ccccc4)nc(-n4c5ccccc5c5c6c7ccccc7n(-c7c(-c8ccccc8)ccc8c7c7ccccc7n8-c7ccccc7)c6ccc54)n3)ccc21]

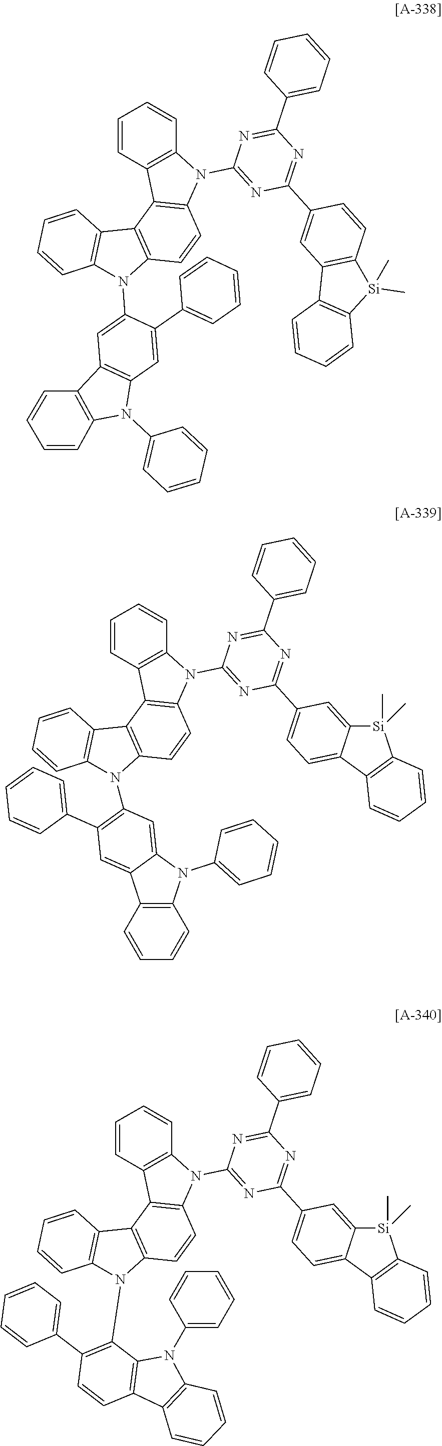 [SMILES: C[Si]1(C)c2ccccc2-c2cc(-c3nc(-c4ccccc4)nc(-n4c5ccccc5c5c6c7ccccc7n(-c7cc8c9ccccc9n(-c9ccccc9)c8cc7-c7ccccc7)c6ccc54)n3)ccc21 ; C[Si]1(C)c2ccccc2-c2ccc(-c3nc(-c4ccccc4)nc(-n4c5ccccc5c5c6c7ccccc7n(-c7cc8c(cc7-c7ccccc7)c7ccccc7n8-c7ccccc7)c6ccc54)n3)cc21 ; C[Si]1(C)c2ccccc2-c2ccc(-c3nc(-c4ccccc4)nc(-n4c5ccccc5c5c6c7ccccc7n(-c7c(-c8ccccc8)ccc8c9ccccc9n(-c9ccccc9)c78)c6ccc54)n3)cc21]

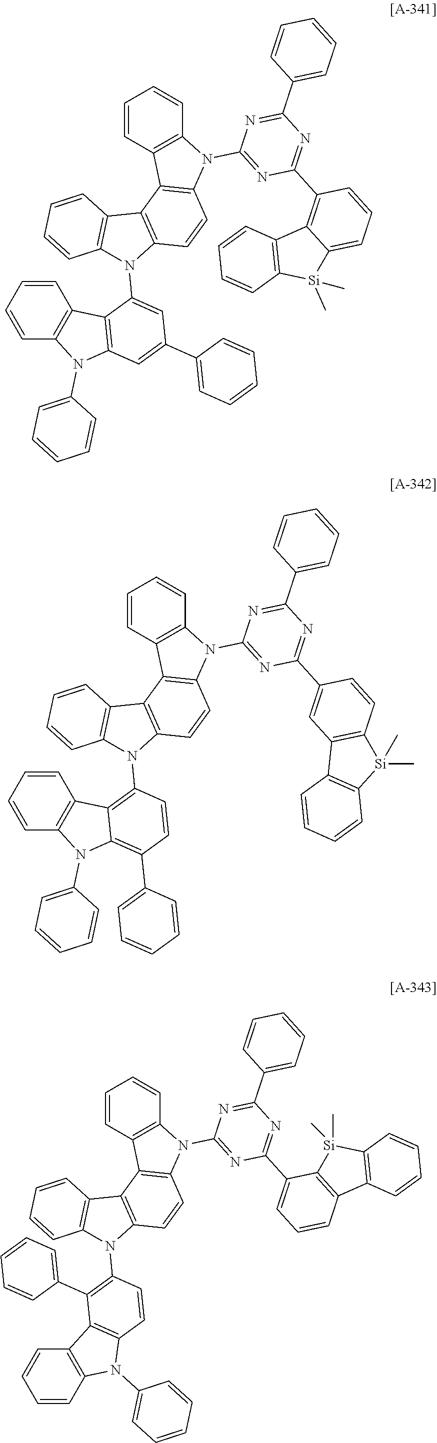 [SMILES: C[Si]1(C)c2ccccc2-c2c(-c3nc(-c4ccccc4)nc(-n4c5ccccc5c5c6c7ccccc7n(-c7cc(-c8ccccc8)cc8c7c7ccccc7n8-c7ccccc7)c6ccc54)n3)cccc21 ; C[Si]1(C)c2ccccc2-c2cc(-c3nc(-c4ccccc4)nc(-n4c5ccccc5c5c6c7ccccc7n(-c7ccc(-c8ccccc8)c8c7c7ccccc7n8-c7ccccc7)c6ccc54)n3)ccc21 ; C[Si]1(C)c2ccccc2-c2cccc(-c3nc(-c4ccccc4)nc(-n4c5ccccc5c5c6c7ccccc7n(-c7ccc8c(c7-c7ccccc7)c7ccccc7n8-c7ccccc7)c6ccc54)n3)c21]

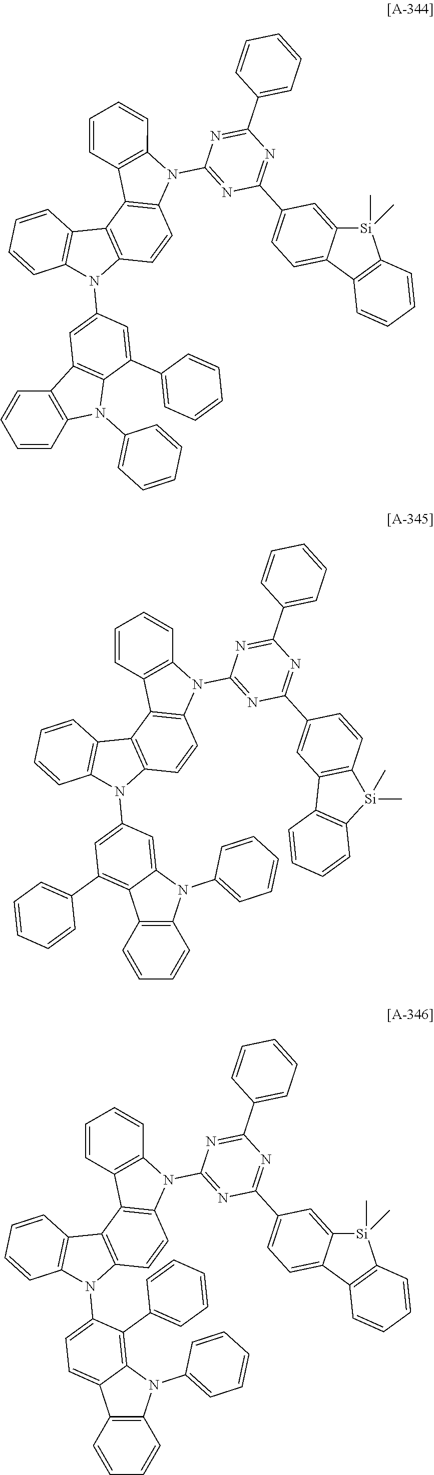 [SMILES: C[Si]1(C)c2ccccc2-c2ccc(-c3nc(-c4ccccc4)nc(-n4c5ccccc5c5c6c7ccccc7n(-c7cc(-c8ccccc8)c8c(c7)c7ccccc7n8-c7ccccc7)c6ccc54)n3)cc21 ; C[Si]1(C)c2ccccc2-c2cc(-c3nc(-c4ccccc4)nc(-n4c5ccccc5c5c6c7ccccc7n(-c7cc(-c8ccccc8)c8c9ccccc9n(-c9ccccc9)c8c7)c6ccc54)n3)ccc21 ; C[Si]1(C)c2ccccc2-c2ccc(-c3nc(-c4ccccc4)nc(-n4c5ccccc5c5c6c7ccccc7n(-c7ccc8c9ccccc9n(-c9ccccc9)c8c7-c7ccccc7)c6ccc54)n3)cc21]

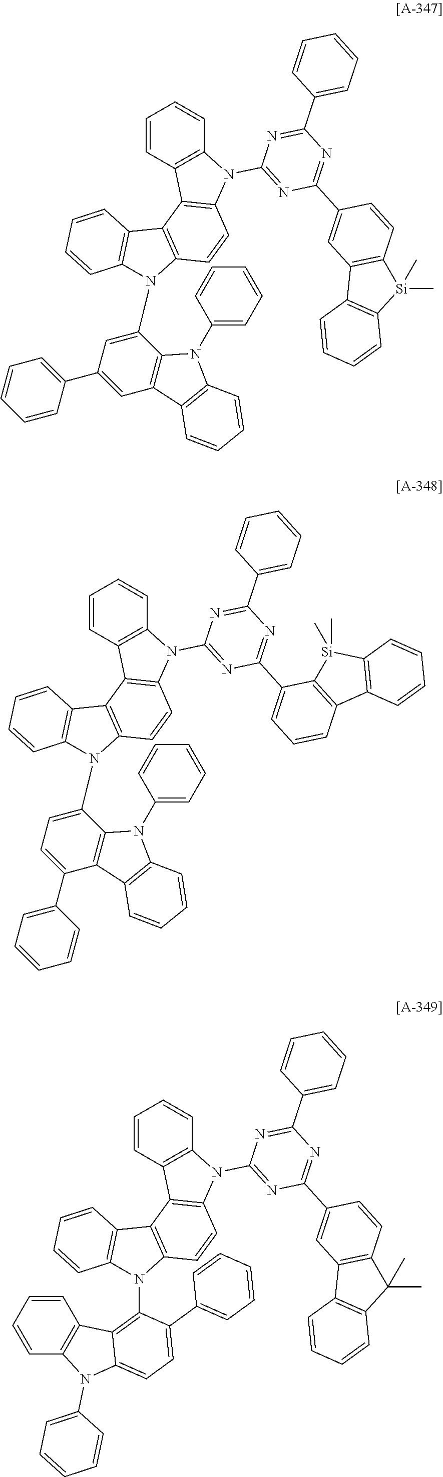 [SMILES: C[Si]1(C)c2ccccc2-c2cc(-c3nc(-c4ccccc4)nc(-n4c5ccccc5c5c6c7ccccc7n(-c7cc(-c8ccccc8)cc8c9ccccc9n(-c9ccccc9)c78)c6ccc54)n3)ccc21 ; C[Si]1(C)c2ccccc2-c2cccc(-c3nc(-c4ccccc4)nc(-n4c5ccccc5c5c6c7ccccc7n(-c7ccc(-c8ccccc8)c8c9ccccc9n(-c9ccccc9)c78)c6ccc54)n3)c21 ; CC1(C)c2ccccc2-c2cc(-c3nc(-c4ccccc4)nc(-n4c5ccccc5c5c6c7ccccc7n(-c7c(-c8ccccc8)ccc8c7c7ccccc7n8-c7ccccc7)c6ccc54)n3)ccc21]

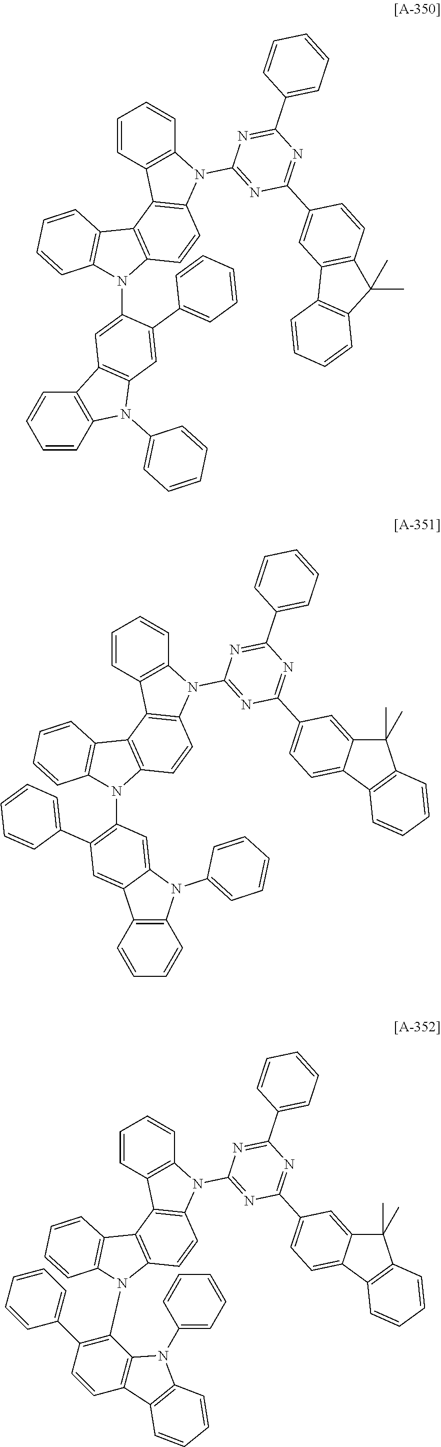 [SMILES: CC1(C)c2ccccc2-c2cc(-c3nc(-c4ccccc4)nc(-n4c5ccccc5c5c6c7ccccc7n(-c7cc8c9ccccc9n(-c9ccccc9)c8cc7-c7ccccc7)c6ccc54)n3)ccc21 ; CC1(C)c2ccccc2-c2ccc(-c3nc(-c4ccccc4)nc(-n4c5ccccc5c5c6c7ccccc7n(-c7cc8c(cc7-c7ccccc7)c7ccccc7n8-c7ccccc7)c6ccc54)n3)cc21 ; CC1(C)c2ccccc2-c2ccc(-c3nc(-c4ccccc4)nc(-n4c5ccccc5c5c6c7ccccc7n(-c7c(-c8ccccc8)ccc8c9ccccc9n(-c9ccccc9)c78)c6ccc54)n3)cc21]

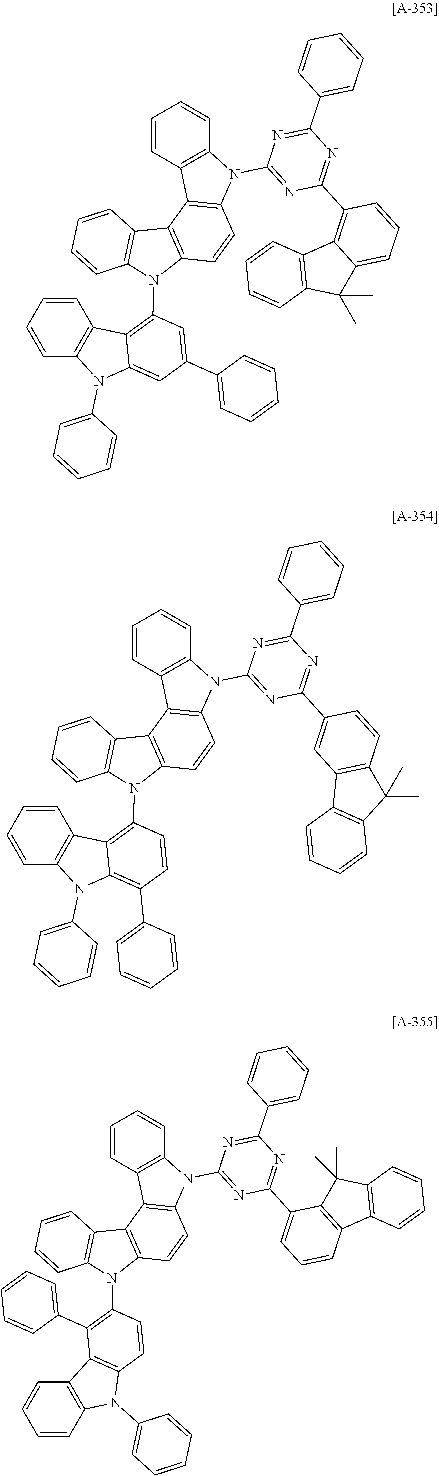 [SMILES: CC1(C)c2ccccc2-c2c(-c3nc(-c4ccccc4)nc(-n4c5ccccc5c5c6c7ccccc7n(-c7cc(-c8ccccc8)cc8c7c7ccccc7n8-c7ccccc7)c6ccc54)n3)cccc21 ; CC1(C)c2ccccc2-c2cc(-c3nc(-c4ccccc4)nc(-n4c5ccccc5c5c6c7ccccc7n(-c7ccc(-c8ccccc8)c8c7c7ccccc7n8-c7ccccc7)c6ccc54)n3)ccc21 ; CC1(C)c2ccccc2-c2cccc(-c3nc(-c4ccccc4)nc(-n4c5ccccc5c5c6c7ccccc7n(-c7ccc8c(c7-c7ccccc7)c7ccccc7n8-c7ccccc7)c6ccc54)n3)c21]

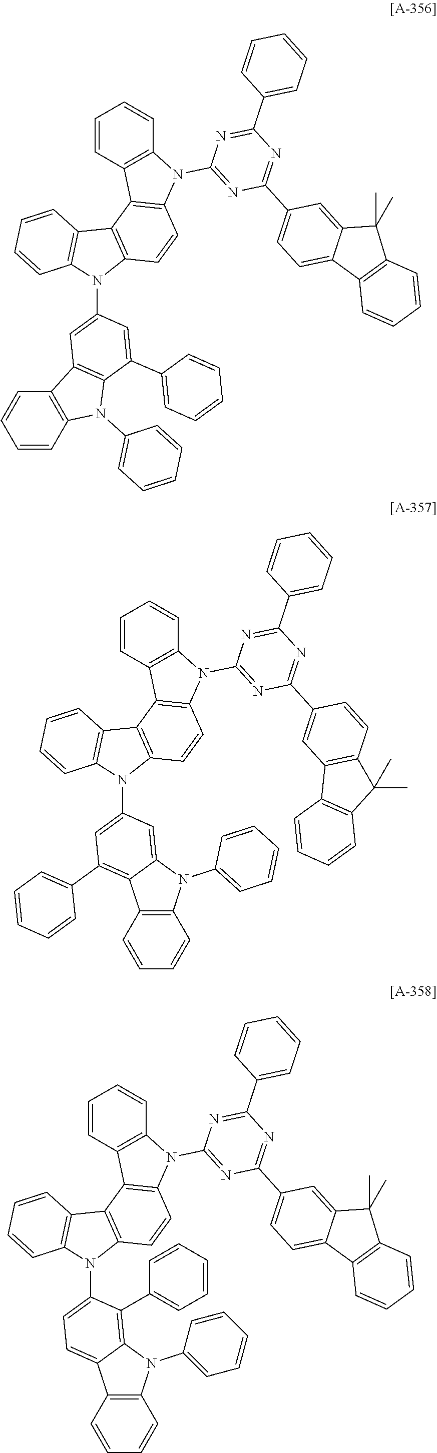 [SMILES: CC1(C)c2ccccc2-c2ccc(-c3nc(-c4ccccc4)nc(-n4c5ccccc5c5c6c7ccccc7n(-c7cc(-c8ccccc8)c8c(c7)c7ccccc7n8-c7ccccc7)c6ccc54)n3)cc21 ; CC1(C)c2ccccc2-c2cc(-c3nc(-c4ccccc4)nc(-n4c5ccccc5c5c6c7ccccc7n(-c7cc(-c8ccccc8)c8c9ccccc9n(-c9ccccc9)c8c7)c6ccc54)n3)ccc21 ; CC1(C)c2ccccc2-c2ccc(-c3nc(-c4ccccc4)nc(-n4c5ccccc5c5c6c7ccccc7n(-c7ccc8c9ccccc9n(-c9ccccc9)c8c7-c7ccccc7)c6ccc54)n3)cc21]

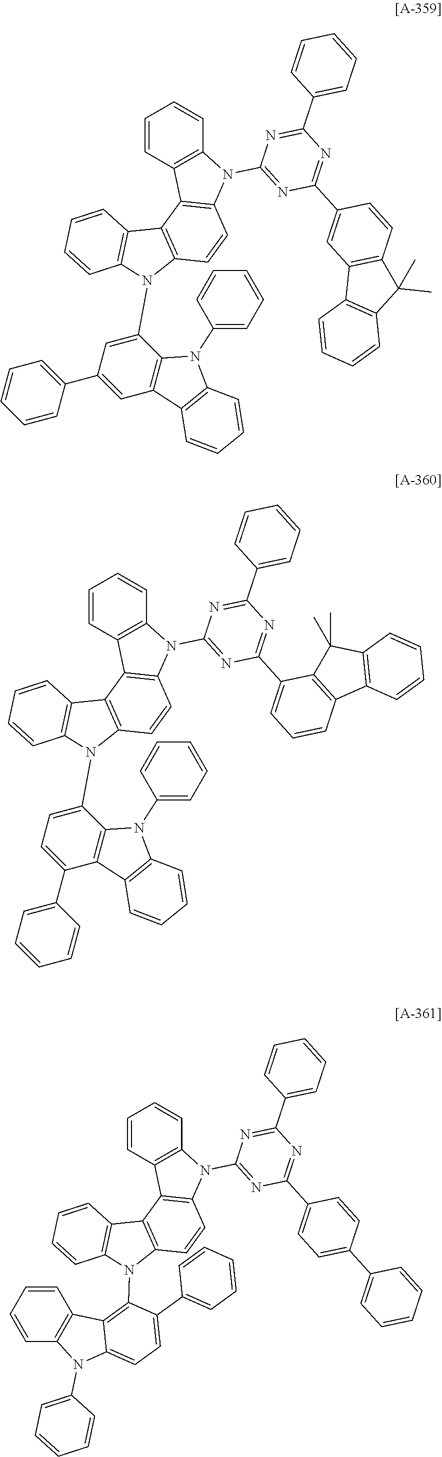 [SMILES: CC1(C)c2ccccc2-c2cc(-c3nc(-c4ccccc4)nc(-n4c5ccccc5c5c6c7ccccc7n(-c7cc(-c8ccccc8)cc8c9ccccc9n(-c9ccccc9)c78)c6ccc54)n3)ccc21 ; CC1(C)c2ccccc2-c2cccc(-c3nc(-c4ccccc4)nc(-n4c5ccccc5c5c6c7ccccc7n(-c7ccc(-c8ccccc8)c8c9ccccc9n(-c9ccccc9)c78)c6ccc54)n3)c21 ; c1ccc(-c2ccc(-c3nc(-c4ccccc4)nc(-n4c5ccccc5c5c6c7ccccc7n(-c7c(-c8ccccc8)ccc8c7c7ccccc7n8-c7ccccc7)c6ccc54)n3)cc2)cc1]

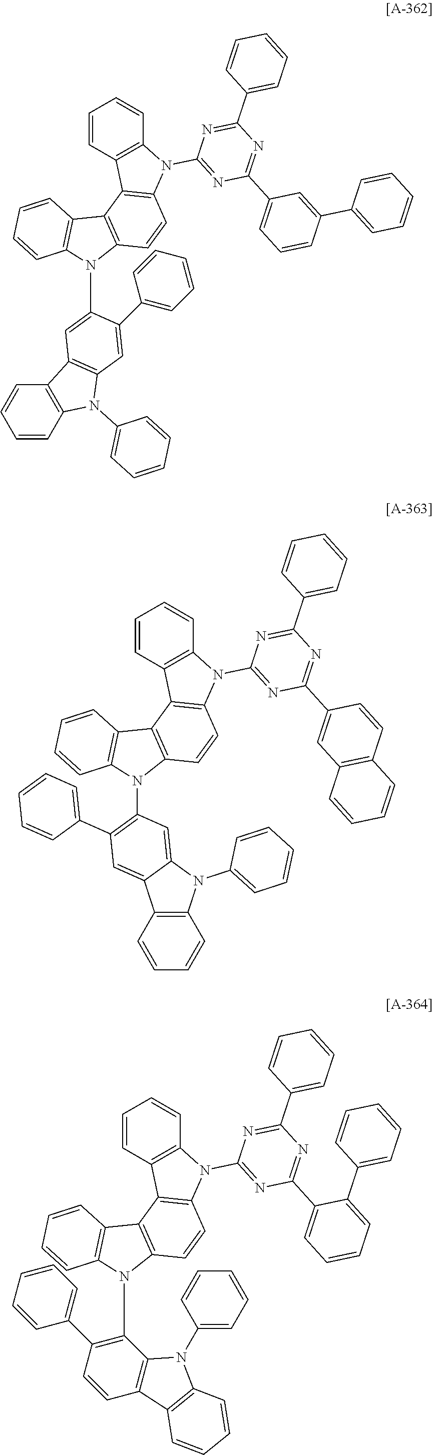 [SMILES: c1ccc(-c2cccc(-c3nc(-c4ccccc4)nc(-n4c5ccccc5c5c6c7ccccc7n(-c7cc8c9ccccc9n(-c9ccccc9)c8cc7-c7ccccc7)c6ccc54)n3)c2)cc1 ; c1ccc(-c2nc(-c3ccc4ccccc4c3)nc(-n3c4ccccc4c4c5c6ccccc6n(-c6cc7c(cc6-c6ccccc6)c6ccccc6n7-c6ccccc6)c5ccc43)n2)cc1 ; c1ccc(-c2nc(-c3ccccc3-c3ccccc3)nc(-n3c4ccccc4c4c5c6ccccc6n(-c6c(-c7ccccc7)ccc7c8ccccc8n(-c8ccccc8)c67)c5ccc43)n2)cc1]

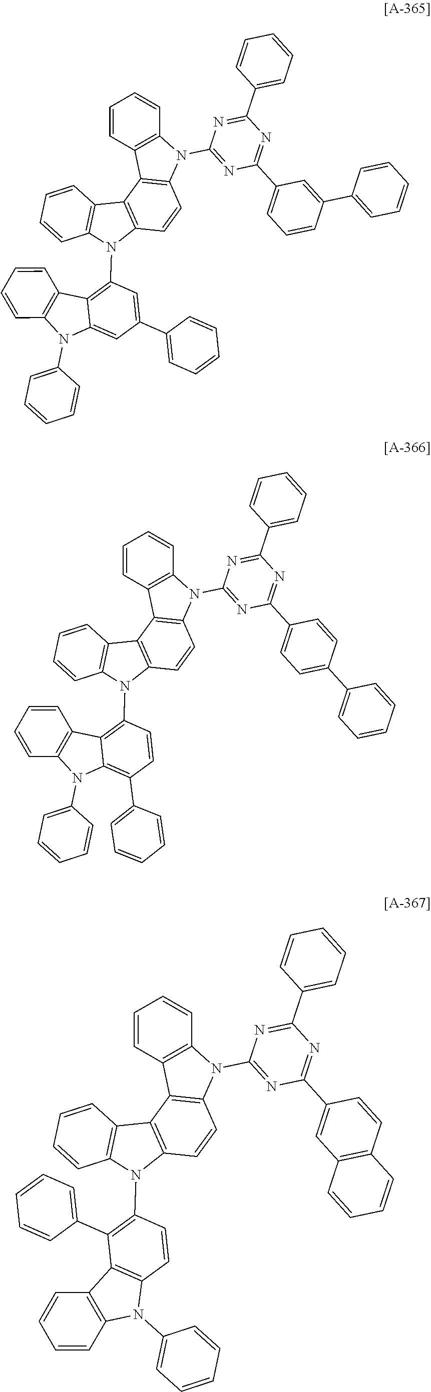 [SMILES: c1ccc(-c2cccc(-c3nc(-c4ccccc4)nc(-n4c5ccccc5c5c6c7ccccc7n(-c7cc(-c8ccccc8)cc8c7c7ccccc7n8-c7ccccc7)c6ccc54)n3)c2)cc1 ; c1ccc(-c2ccc(-c3nc(-c4ccccc4)nc(-n4c5ccccc5c5c6c7ccccc7n(-c7ccc(-c8ccccc8)c8c7c7ccccc7n8-c7ccccc7)c6ccc54)n3)cc2)cc1 ; c1ccc(-c2nc(-c3ccc4ccccc4c3)nc(-n3c4ccccc4c4c5c6ccccc6n(-c6ccc7c(c6-c6ccccc6)c6ccccc6n7-c6ccccc6)c5ccc43)n2)cc1]

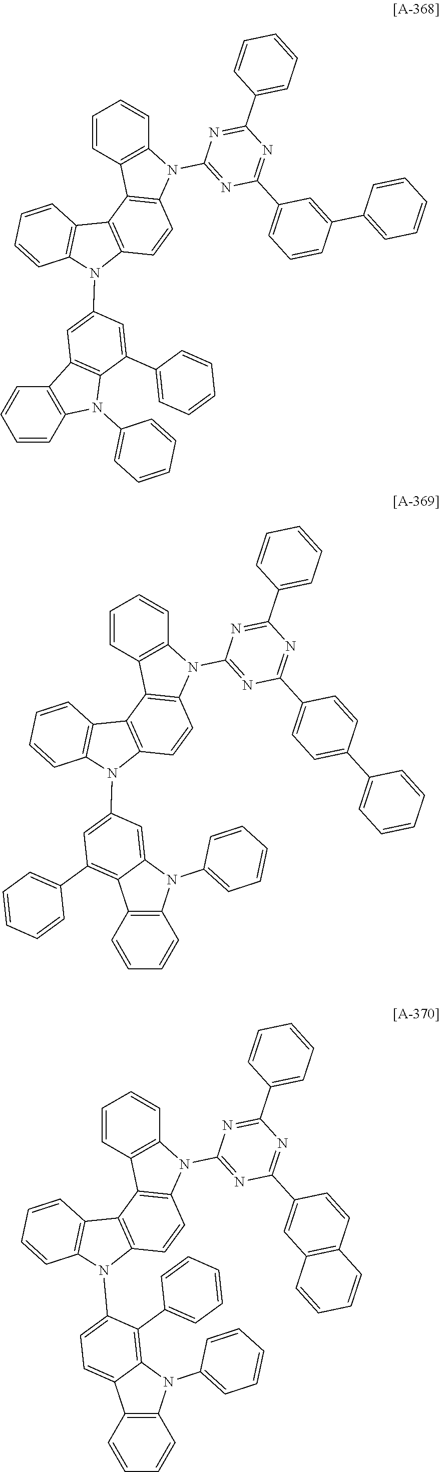 [SMILES: c1ccc(-c2cccc(-c3nc(-c4ccccc4)nc(-n4c5ccccc5c5c6c7ccccc7n(-c7cc(-c8ccccc8)c8c(c7)c7ccccc7n8-c7ccccc7)c6ccc54)n3)c2)cc1 ; c1ccc(-c2ccc(-c3nc(-c4ccccc4)nc(-n4c5ccccc5c5c6c7ccccc7n(-c7cc(-c8ccccc8)c8c9ccccc9n(-c9ccccc9)c8c7)c6ccc54)n3)cc2)cc1 ; c1ccc(-c2nc(-c3ccc4ccccc4c3)nc(-n3c4ccccc4c4c5c6ccccc6n(-c6ccc7c8ccccc8n(-c8ccccc8)c7c6-c6ccccc6)c5ccc43)n2)cc1]

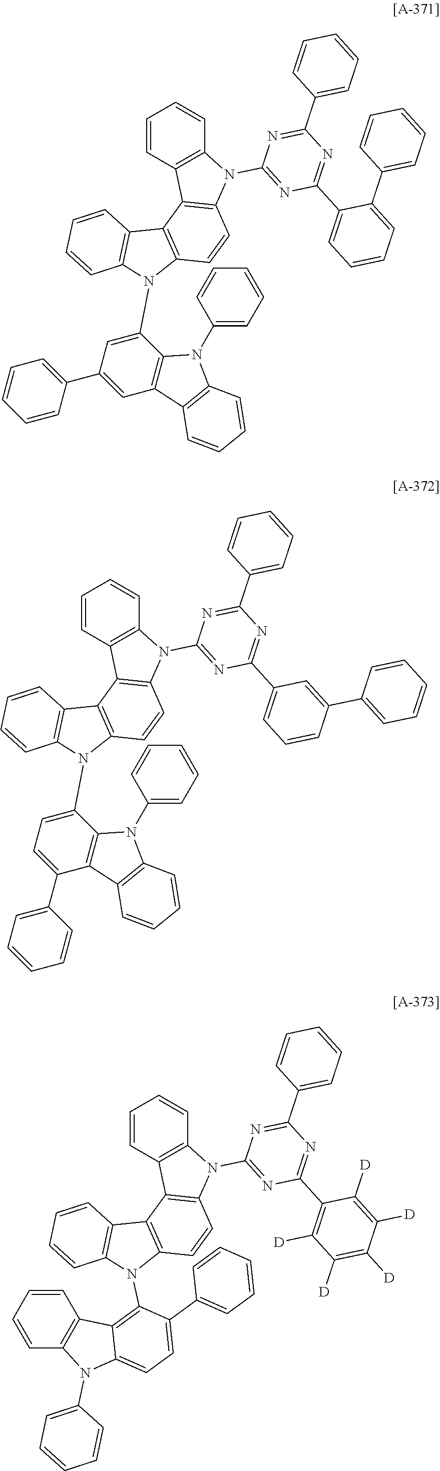 [SMILES: c1ccc(-c2cc(-n3c4ccccc4c4c5c6ccccc6n(-c6nc(-c7ccccc7)nc(-c7ccccc7-c7ccccc7)n6)c5ccc43)c3c(c2)c2ccccc2n3-c2ccccc2)cc1 ; c1ccc(-c2cccc(-c3nc(-c4ccccc4)nc(-n4c5ccccc5c5c6c7ccccc7n(-c7ccc(-c8ccccc8)c8c9ccccc9n(-c9ccccc9)c78)c6ccc54)n3)c2)cc1 ; [2H]c1c([2H])c([2H])c(-c2nc(-c3ccccc3)nc(-n3c4ccccc4c4c5c6ccccc6n(-c6c(-c7ccccc7)ccc7c6c6ccccc6n7-c6ccccc6)c5ccc43)n2)c([2H])c1[2H]]

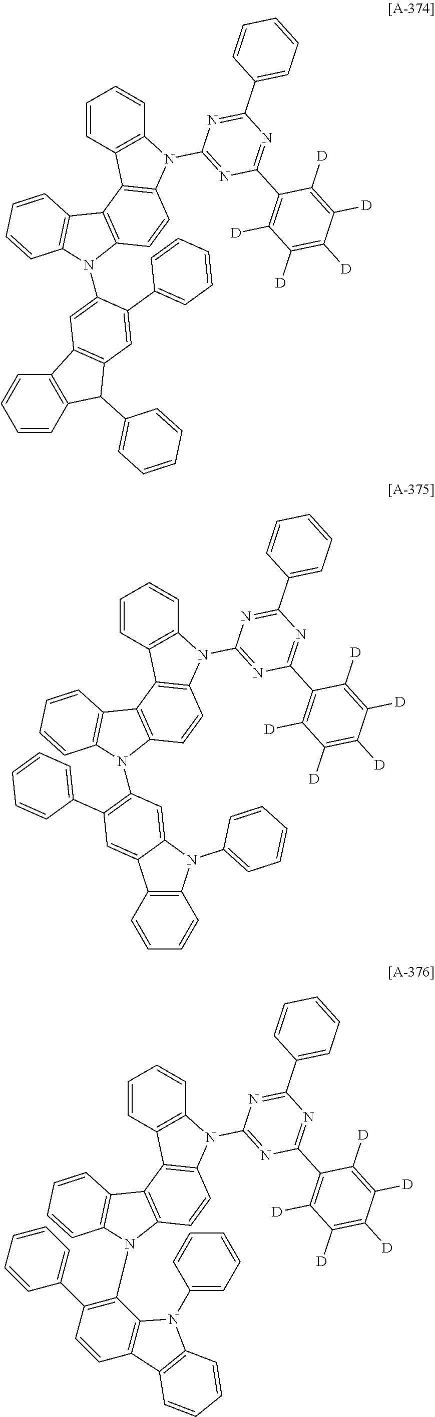 [SMILES: [2H]c1c([2H])c([2H])c(-c2nc(-c3ccccc3)nc(-n3c4ccccc4c4c5c6ccccc6n(-c6cc7c(cc6-c6ccccc6)C(c6ccccc6)c6ccccc6-7)c5ccc43)n2)c([2H])c1[2H] ; [2H]c1c([2H])c([2H])c(-c2nc(-c3ccccc3)nc(-n3c4ccccc4c4c5c6ccccc6n(-c6cc7c(cc6-c6ccccc6)c6ccccc6n7-c6ccccc6)c5ccc43)n2)c([2H])c1[2H] ; [2H]c1c([2H])c([2H])c(-c2nc(-c3ccccc3)nc(-n3c4ccccc4c4c5c6ccccc6n(-c6c(-c7ccccc7)ccc7c8ccccc8n(-c8ccccc8)c67)c5ccc43)n2)c([2H])c1[2H]]

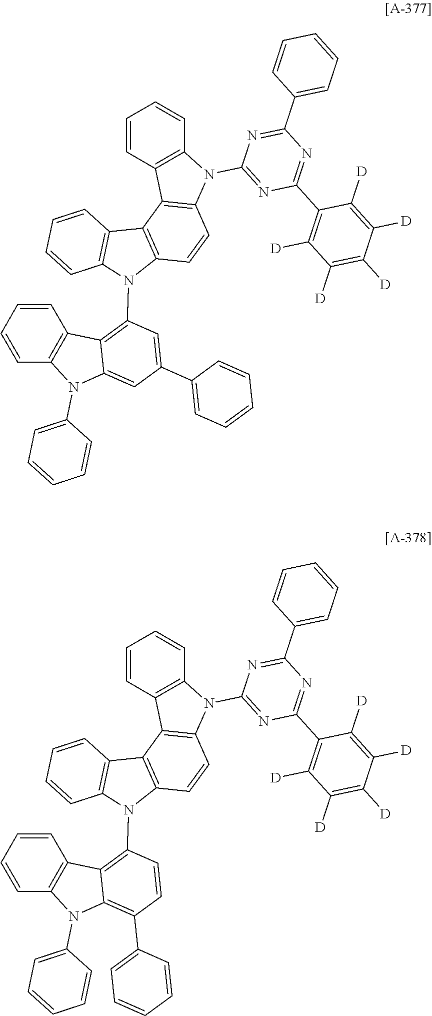 [SMILES: [2H]c1c([2H])c([2H])c(-c2nc(-c3ccccc3)nc(-n3c4ccccc4c4c5c6ccccc6n(-c6cc(-c7ccccc7)cc7c6c6ccccc6n7-c6ccccc6)c5ccc43)n2)c([2H])c1[2H] ; [2H]c1c([2H])c([2H])c(-c2nc(-c3ccccc3)nc(-n3c4ccccc4c4c5c6ccccc6n(-c6ccc(-c7ccccc7)c7c6c6ccccc6n7-c6ccccc6)c5ccc43)n2)c([2H])c1[2H]]

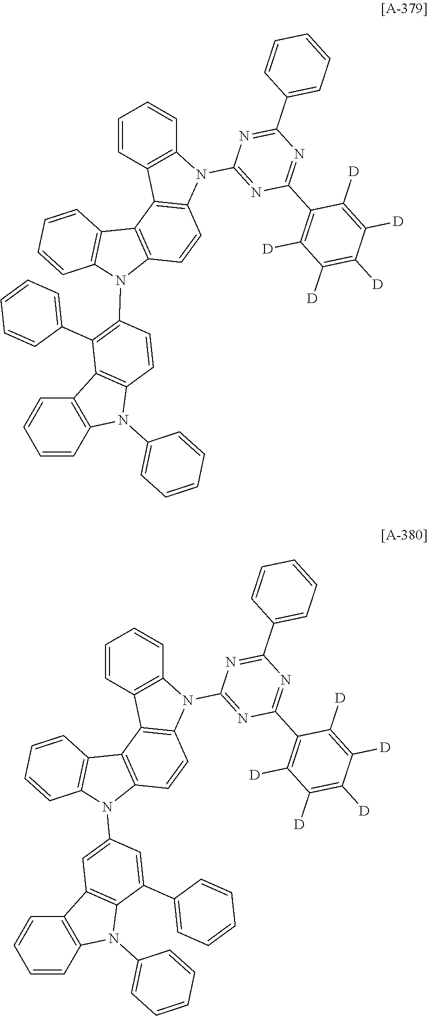 [SMILES: [2H]c1c([2H])c([2H])c(-c2nc(-c3ccccc3)nc(-n3c4ccccc4c4c5c6ccccc6n(-c6ccc7c(c6-c6ccccc6)c6ccccc6n7-c6ccccc6)c5ccc43)n2)c([2H])c1[2H] ; [2H]c1c([2H])c([2H])c(-c2nc(-c3ccccc3)nc(-n3c4ccccc4c4c5c6ccccc6n(-c6cc(-c7ccccc7)c7c(c6)c6ccccc6n7-c6ccccc6)c5ccc43)n2)c([2H])c1[2H]]

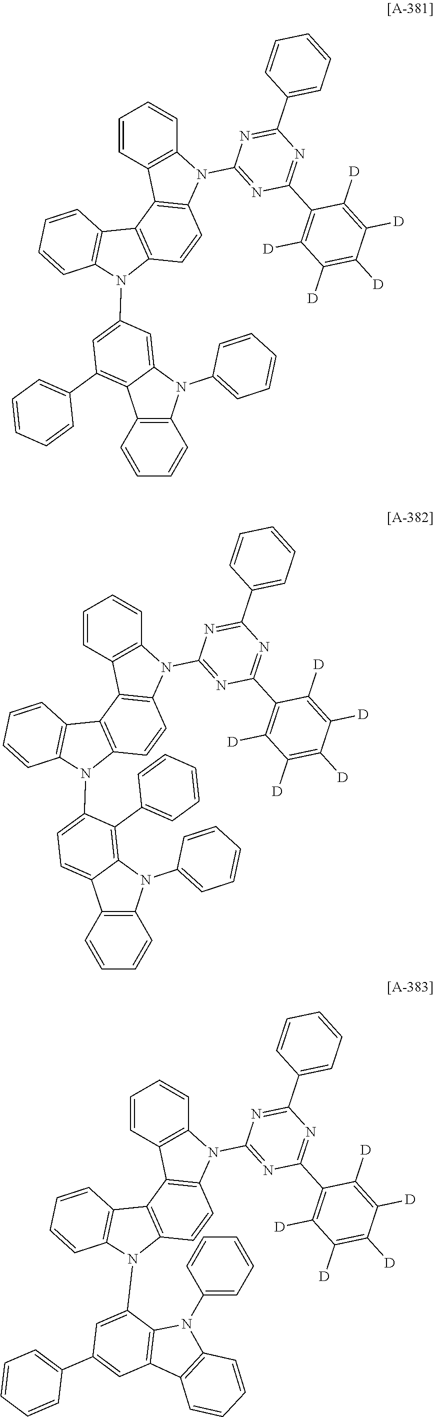 [SMILES: [2H]c1c([2H])c([2H])c(-c2nc(-c3ccccc3)nc(-n3c4ccccc4c4c5c6ccccc6n(-c6cc(-c7ccccc7)c7c8ccccc8n(-c8ccccc8)c7c6)c5ccc43)n2)c([2H])c1[2H] ; [2H]c1c([2H])c([2H])c(-c2nc(-c3ccccc3)nc(-n3c4ccccc4c4c5c6ccccc6n(-c6ccc7c8ccccc8n(-c8ccccc8)c7c6-c6ccccc6)c5ccc43)n2)c([2H])c1[2H] ; [2H]c1c([2H])c([2H])c(-c2nc(-c3ccccc3)nc(-n3c4ccccc4c4c5c6ccccc6n(-c6cc(-c7ccccc7)cc7c8ccccc8n(-c8ccccc8)c67)c5ccc43)n2)c([2H])c1[2H]]

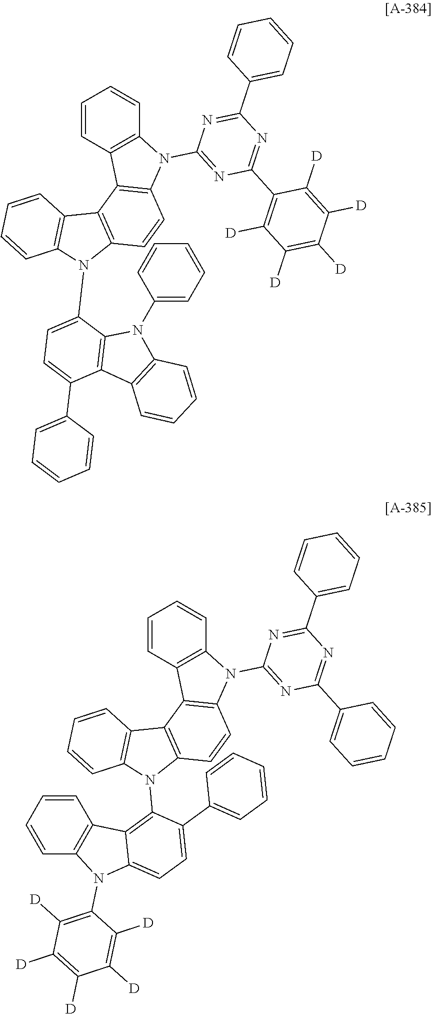 [SMILES: [2H]c1c([2H])c([2H])c(-c2nc(-c3ccccc3)nc(-n3c4ccccc4c4c5c6ccccc6n(-c6ccc(-c7ccccc7)c7c8ccccc8n(-c8ccccc8)c67)c5ccc43)n2)c([2H])c1[2H] ; [2H]c1c([2H])c([2H])c(-n2c3ccccc3c3c(-n4c5ccccc5c5c6c7ccccc7n(-c7nc(-c8ccccc8)nc(-c8ccccc8)n7)c6ccc54)c(-c4ccccc4)ccc32)c([2H])c1[2H]]

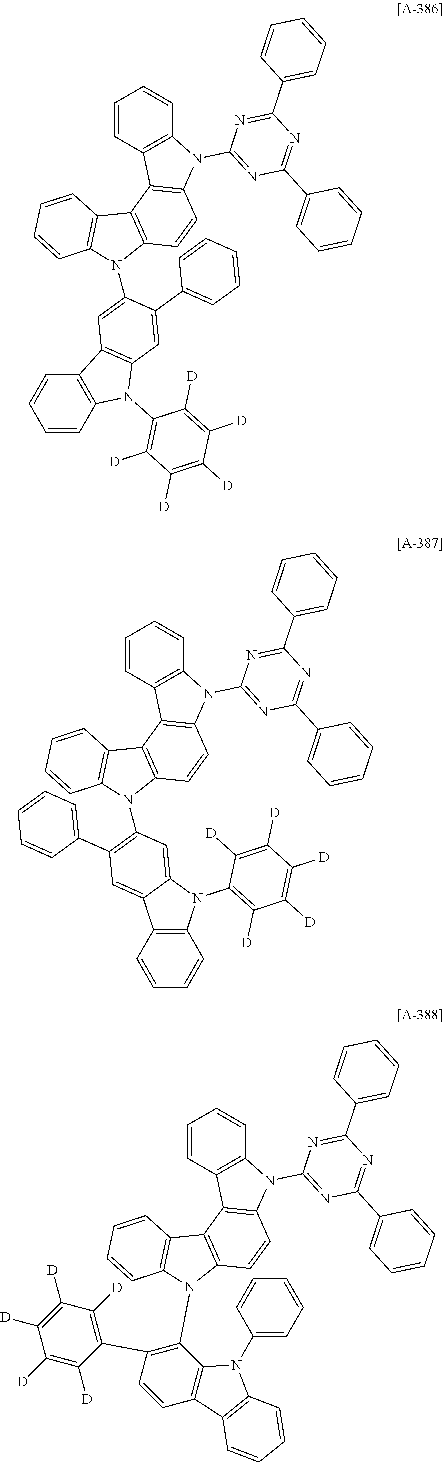 [SMILES: [2H]c1c([2H])c([2H])c(-n2c3ccccc3c3cc(-n4c5ccccc5c5c6c7ccccc7n(-c7nc(-c8ccccc8)nc(-c8ccccc8)n7)c6ccc54)c(-c4ccccc4)cc32)c([2H])c1[2H] ; [2H]c1c([2H])c([2H])c(-n2c3ccccc3c3cc(-c4ccccc4)c(-n4c5ccccc5c5c6c7ccccc7n(-c7nc(-c8ccccc8)nc(-c8ccccc8)n7)c6ccc54)cc32)c([2H])c1[2H] ; [2H]c1c([2H])c([2H])c(-c2ccc3c4ccccc4n(-c4ccccc4)c3c2-n2c3ccccc3c3c4c5ccccc5n(-c5nc(-c6ccccc6)nc(-c6ccccc6)n5)c4ccc32)c([2H])c1[2H]]

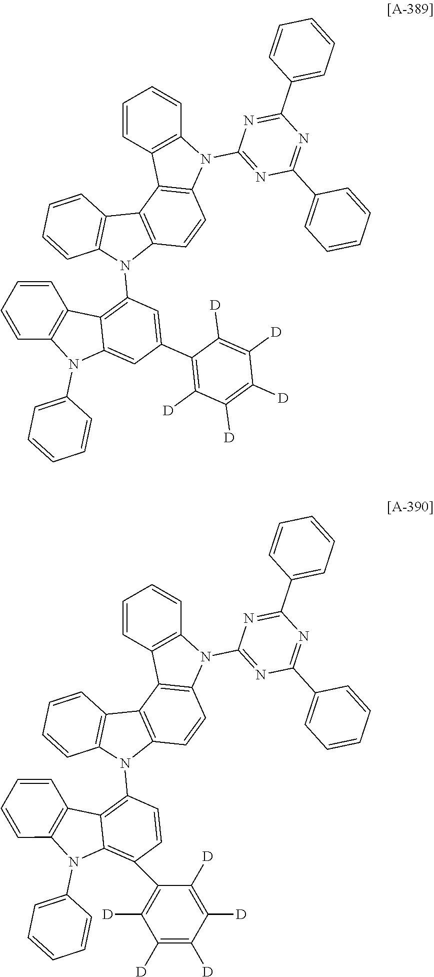 [SMILES: [2H]c1c([2H])c([2H])c(-c2cc(-n3c4ccccc4c4c5c6ccccc6n(-c6nc(-c7ccccc7)nc(-c7ccccc7)n6)c5ccc43)c3c4ccccc4n(-c4ccccc4)c3c2)c([2H])c1[2H] ; [2H]c1c([2H])c([2H])c(-c2ccc(-n3c4ccccc4c4c5c6ccccc6n(-c6nc(-c7ccccc7)nc(-c7ccccc7)n6)c5ccc43)c3c4ccccc4n(-c4ccccc4)c23)c([2H])c1[2H]]

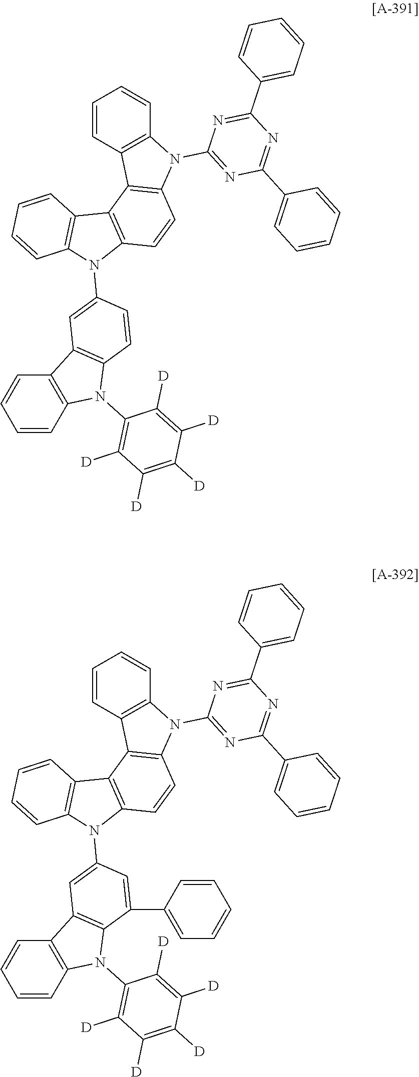 [SMILES: [2H]c1c([2H])c([2H])c(-n2c3ccccc3c3cc(-n4c5ccccc5c5c6c7ccccc7n(-c7nc(-c8ccccc8)nc(-c8ccccc8)n7)c6ccc54)ccc32)c([2H])c1[2H] ; [2H]c1c([2H])c([2H])c(-n2c3ccccc3c3cc(-n4c5ccccc5c5c6c7ccccc7n(-c7nc(-c8ccccc8)nc(-c8ccccc8)n7)c6ccc54)cc(-c4ccccc4)c32)c([2H])c1[2H]]

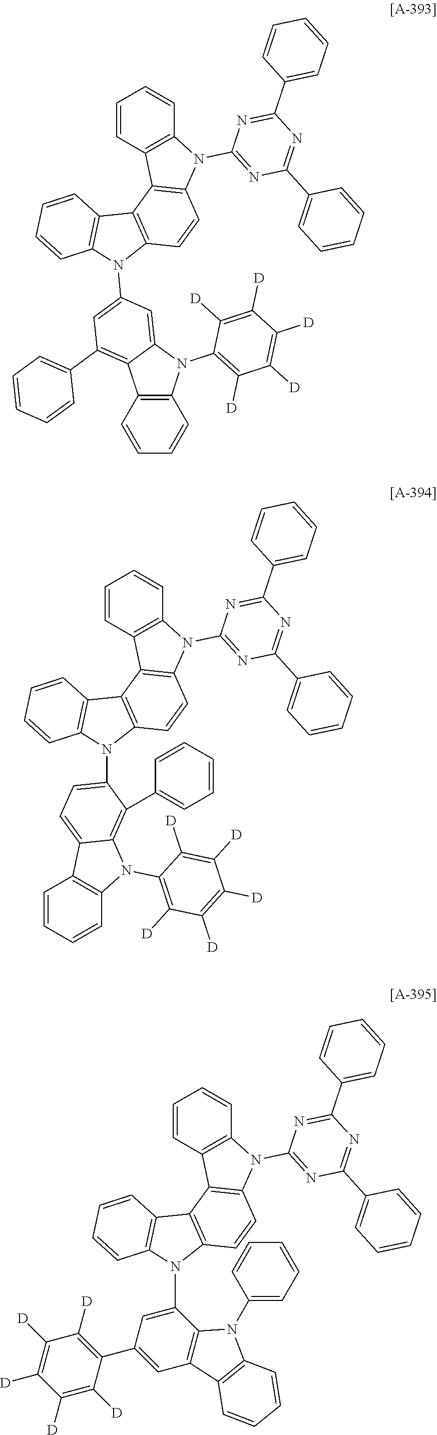 [SMILES: [2H]c1c([2H])c([2H])c(-n2c3ccccc3c3c(-c4ccccc4)cc(-n4c5ccccc5c5c6c7ccccc7n(-c7nc(-c8ccccc8)nc(-c8ccccc8)n7)c6ccc54)cc32)c([2H])c1[2H] ; [2H]c1c([2H])c([2H])c(-n2c3ccccc3c3ccc(-n4c5ccccc5c5c6c7ccccc7n(-c7nc(-c8ccccc8)nc(-c8ccccc8)n7)c6ccc54)c(-c4ccccc4)c32)c([2H])c1[2H] ; [2H]c1c([2H])c([2H])c(-c2cc(-n3c4ccccc4c4c5c6ccccc6n(-c6nc(-c7ccccc7)nc(-c7ccccc7)n6)c5ccc43)c3c(c2)c2ccccc2n3-c2ccccc2)c([2H])c1[2H]]

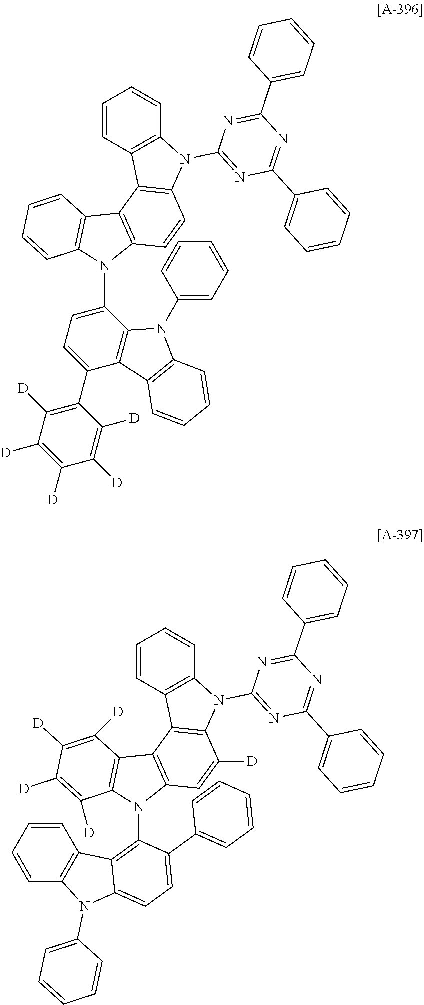 [SMILES: [2H]c1c([2H])c([2H])c(-c2ccc(-n3c4ccccc4c4c5c6ccccc6n(-c6nc(-c7ccccc7)nc(-c7ccccc7)n6)c5ccc43)c3c2c2ccccc2n3-c2ccccc2)c([2H])c1[2H] ; [2H]c1c([2H])c([2H])c2c(c1[2H])c1c3c4ccccc4n(-c4nc(-c5ccccc5)nc(-c5ccccc5)n4)c3c([2H])cc1n2-c1c(-c2ccccc2)ccc2c1c1ccccc1n2-c1ccccc1]

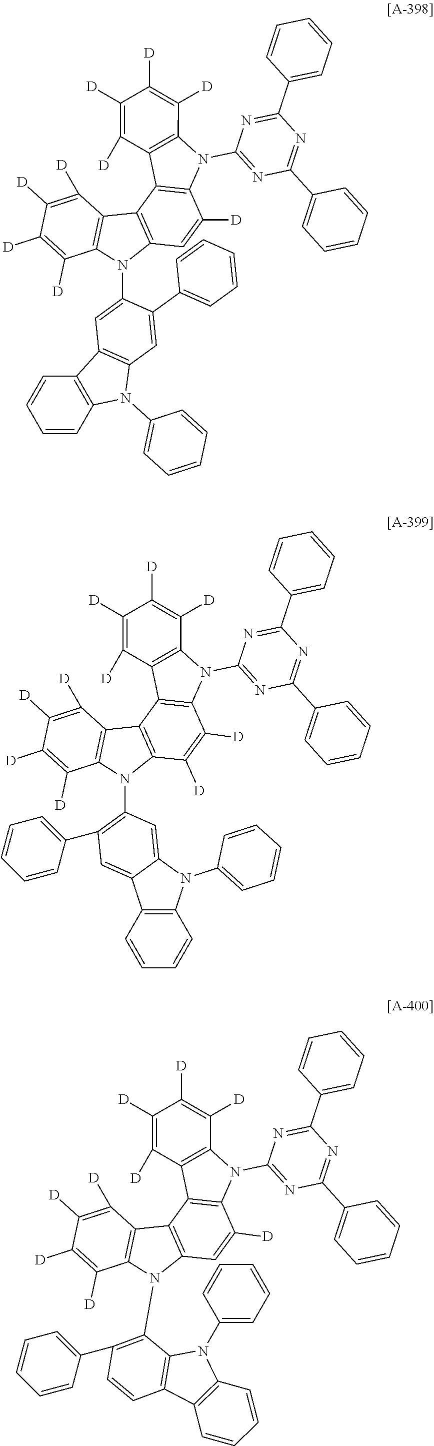 [SMILES: [2H]c1c([2H])c([2H])c2c(c1[2H])c1c3c4c([2H])c([2H])c([2H])c([2H])c4n(-c4nc(-c5ccccc5)nc(-c5ccccc5)n4)c3c([2H])cc1n2-c1cc2c3ccccc3n(-c3ccccc3)c2cc1-c1ccccc1 ; [2H]c1c([2H])c([2H])c2c(c1[2H])c1c3c4c([2H])c([2H])c([2H])c([2H])c4n(-c4cc5c(cc4-c4ccccc4)c4ccccc4n5-c4ccccc4)c3c([2H])c([2H])c1n2-c1nc(-c2ccccc2)nc(-c2ccccc2)n1 ; [2H]c1c([2H])c([2H])c2c(c1[2H])c1c3c4c([2H])c([2H])c([2H])c([2H])c4n(-c4nc(-c5ccccc5)nc(-c5ccccc5)n4)c3c([2H])cc1n2-c1c(-c2ccccc2)ccc2c3ccccc3n(-c3ccccc3)c12]

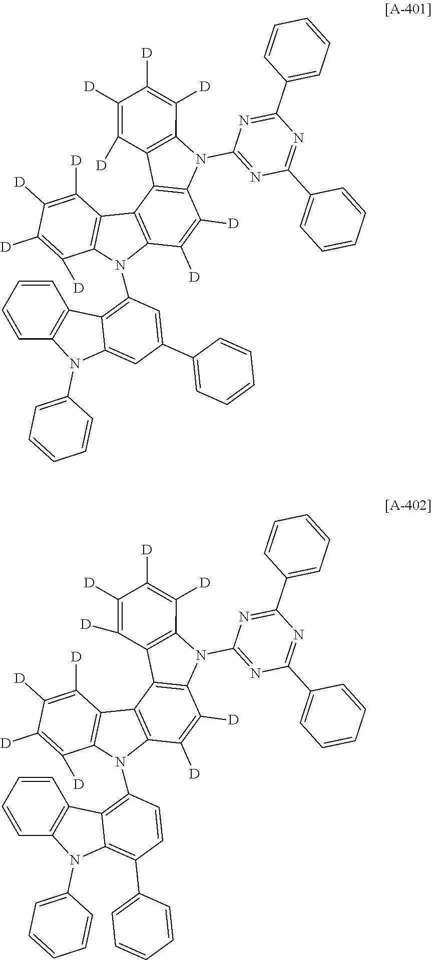 [SMILES: [2H]c1c([2H])c([2H])c2c(c1[2H])c1c3c4c([2H])c([2H])c([2H])c([2H])c4n(-c4cc(-c5ccccc5)cc5c4c4ccccc4n5-c4ccccc4)c3c([2H])c([2H])c1n2-c1nc(-c2ccccc2)nc(-c2ccccc2)n1 ; [2H]c1c([2H])c([2H])c2c(c1[2H])c1c3c4c([2H])c([2H])c([2H])c([2H])c4n(-c4ccc(-c5ccccc5)c5c4c4ccccc4n5-c4ccccc4)c3c([2H])c([2H])c1n2-c1nc(-c2ccccc2)nc(-c2ccccc2)n1]

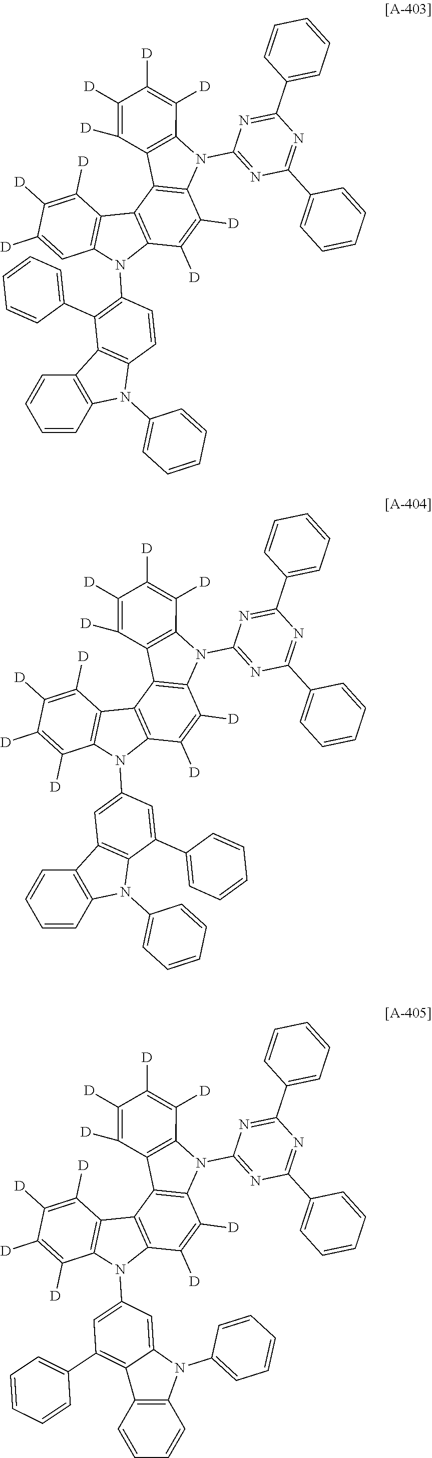 [SMILES: [2H]c1cc2c(c([2H])c1[2H])c1c3c4c([2H])c([2H])c([2H])c([2H])c4n(-c4nc(-c5ccccc5)nc(-c5ccccc5)n4)c3c([2H])c([2H])c1n2-c1ccc2c(c1-c1ccccc1)c1ccccc1n2-c1ccccc1 ; [2H]c1c([2H])c([2H])c2c(c1[2H])c1c3c4c([2H])c([2H])c([2H])c([2H])c4n(-c4nc(-c5ccccc5)nc(-c5ccccc5)n4)c3c([2H])c([2H])c1n2-c1cc(-c2ccccc2)c2c(c1)c1ccccc1n2-c1ccccc1 ; [2H]c1c([2H])c([2H])c2c(c1[2H])c1c3c4c([2H])c([2H])c([2H])c([2H])c4n(-c4nc(-c5ccccc5)nc(-c5ccccc5)n4)c3c([2H])c([2H])c1n2-c1cc(-c2ccccc2)c2c3ccccc3n(-c3ccccc3)c2c1]

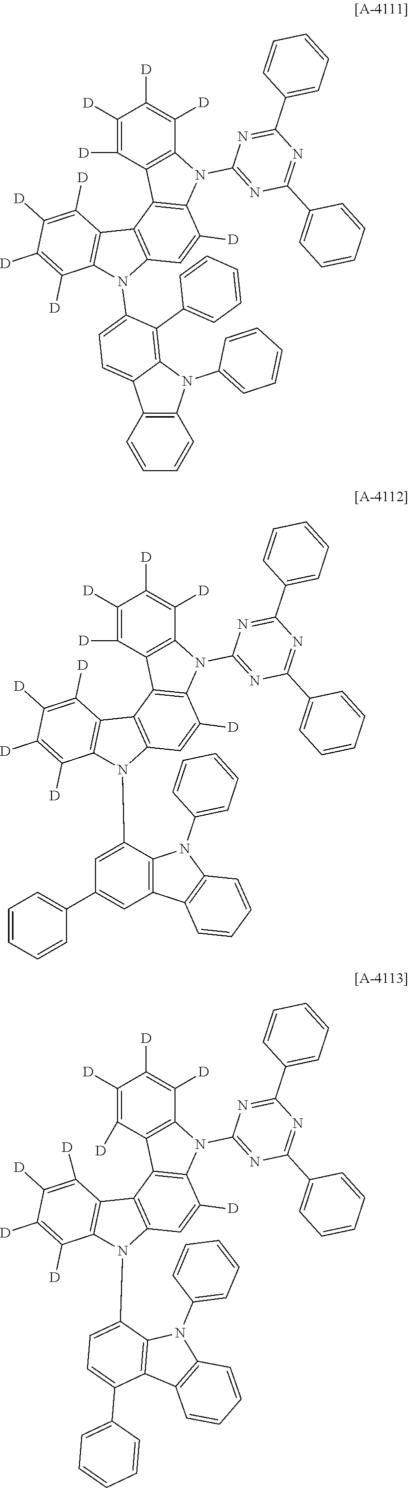 [SMILES: [2H]c1c([2H])c([2H])c2c(c1[2H])c1c3c4c([2H])c([2H])c([2H])c([2H])c4n(-c4nc(-c5ccccc5)nc(-c5ccccc5)n4)c3c([2H])cc1n2-c1ccc2c3ccccc3n(-c3ccccc3)c2c1-c1ccccc1 ; [2H]c1c([2H])c([2H])c2c(c1[2H])c1c3c4c([2H])c([2H])c([2H])c([2H])c4n(-c4nc(-c5ccccc5)nc(-c5ccccc5)n4)c3c([2H])cc1n2-c1cc(-c2ccccc2)cc2c3ccccc3n(-c3ccccc3)c12 ; [2H]c1c([2H])c([2H])c2c(c1[2H])c1c3c4c([2H])c([2H])c([2H])c([2H])c4n(-c4nc(-c5ccccc5)nc(-c5ccccc5)n4)c3c([2H])cc1n2-c1ccc(-c2ccccc2)c2c3ccccc3n(-c3ccccc3)c12]

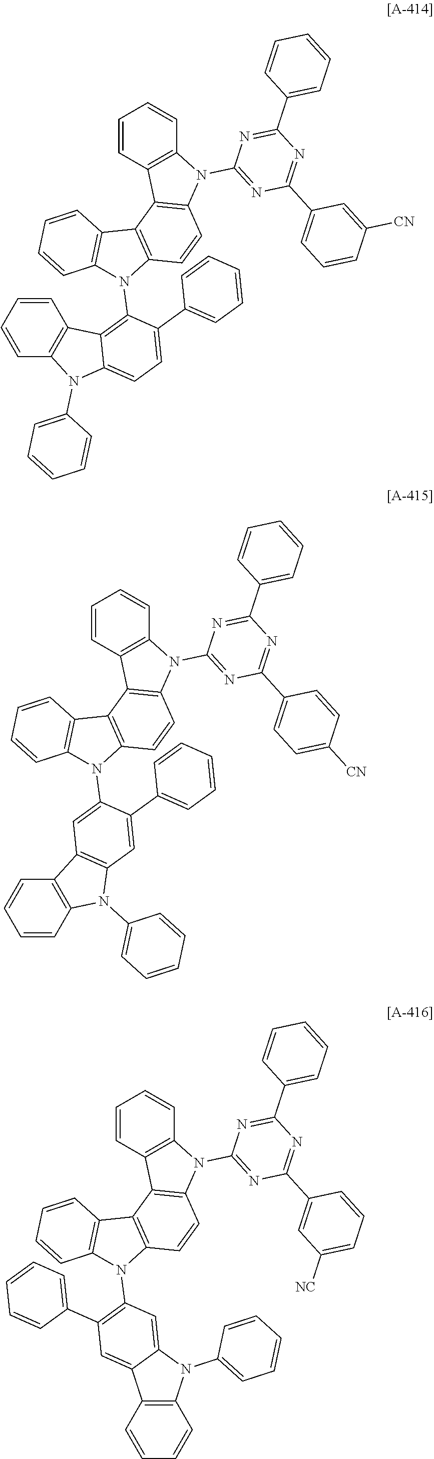 [SMILES: N#Cc1cccc(-c2nc(-c3ccccc3)nc(-n3c4ccccc4c4c5c6ccccc6n(-c6c(-c7ccccc7)ccc7c6c6ccccc6n7-c6ccccc6)c5ccc43)n2)c1 ; N#Cc1ccc(-c2nc(-c3ccccc3)nc(-n3c4ccccc4c4c5c6ccccc6n(-c6cc7c8ccccc8n(-c8ccccc8)c7cc6-c6ccccc6)c5ccc43)n2)cc1 ; N#Cc1cccc(-c2nc(-c3ccccc3)nc(-n3c4ccccc4c4c5c6ccccc6n(-c6cc7c(cc6-c6ccccc6)c6ccccc6n7-c6ccccc6)c5ccc43)n2)c1]

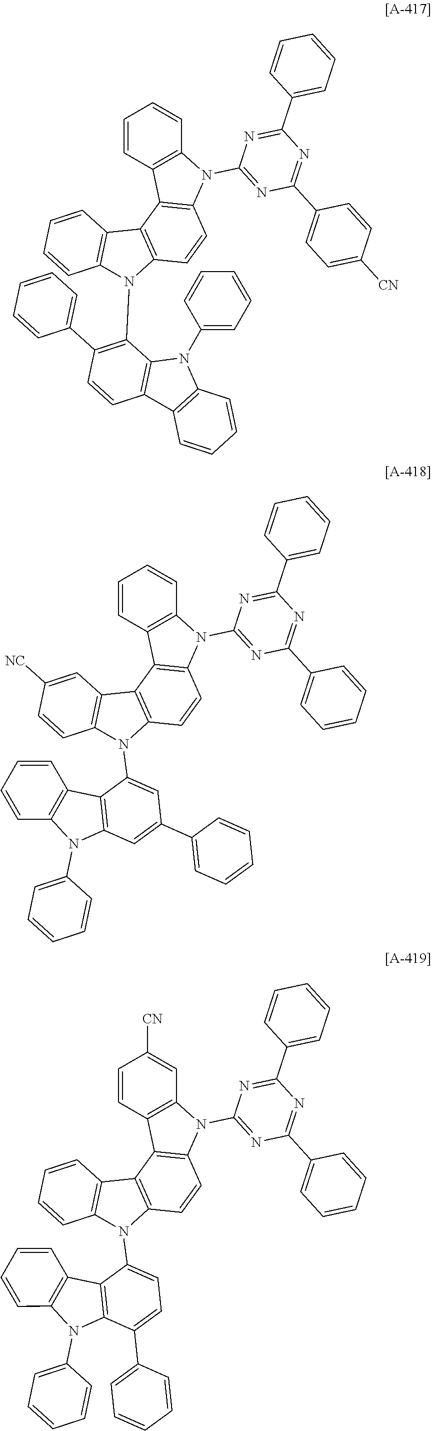 [SMILES: N#Cc1ccc(-c2nc(-c3ccccc3)nc(-n3c4ccccc4c4c5c6ccccc6n(-c6c(-c7ccccc7)ccc7c8ccccc8n(-c8ccccc8)c67)c5ccc43)n2)cc1 ; N#Cc1ccc2c(c1)c1c3c4ccccc4n(-c4nc(-c5ccccc5)nc(-c5ccccc5)n4)c3ccc1n2-c1cc(-c2ccccc2)cc2c1c1ccccc1n2-c1ccccc1 ; N#Cc1ccc2c3c4c5ccccc5n(-c5ccc(-c6ccccc6)c6c5c5ccccc5n6-c5ccccc5)c4ccc3n(-c3nc(-c4ccccc4)nc(-c4ccccc4)n3)c2c1]

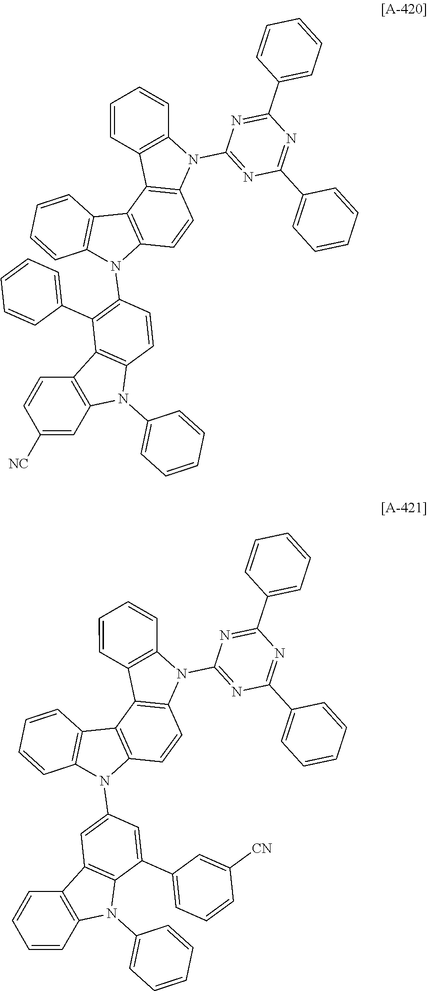 [SMILES: N#Cc1ccc2c3c(-c4ccccc4)c(-n4c5ccccc5c5c6c7ccccc7n(-c7nc(-c8ccccc8)nc(-c8ccccc8)n7)c6ccc54)ccc3n(-c3ccccc3)c2c1 ; N#Cc1cccc(-c2cc(-n3c4ccccc4c4c5c6ccccc6n(-c6nc(-c7ccccc7)nc(-c7ccccc7)n6)c5ccc43)cc3c4ccccc4n(-c4ccccc4)c23)c1]

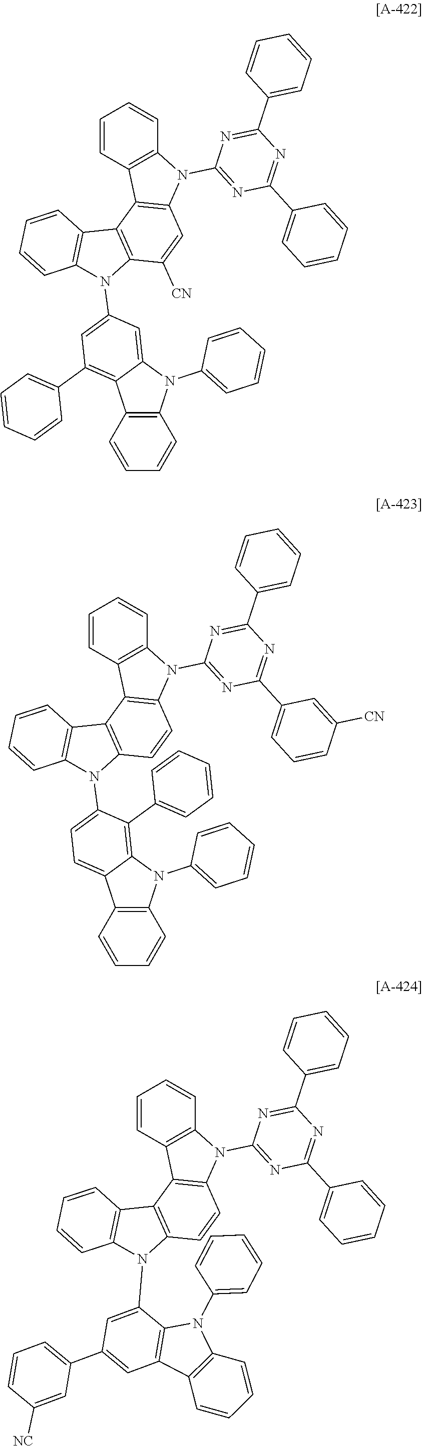 [SMILES: N#Cc1cc2c(c3ccccc3n2-c2nc(-c3ccccc3)nc(-c3ccccc3)n2)c2c3ccccc3n(-c3cc(-c4ccccc4)c4c5ccccc5n(-c5ccccc5)c4c3)c12 ; N#Cc1cccc(-c2nc(-c3ccccc3)nc(-n3c4ccccc4c4c5c6ccccc6n(-c6ccc7c8ccccc8n(-c8ccccc8)c7c6-c6ccccc6)c5ccc43)n2)c1 ; N#Cc1cccc(-c2cc(-n3c4ccccc4c4c5c6ccccc6n(-c6nc(-c7ccccc7)nc(-c7ccccc7)n6)c5ccc43)c3c(c2)c2ccccc2n3-c2ccccc2)c1]

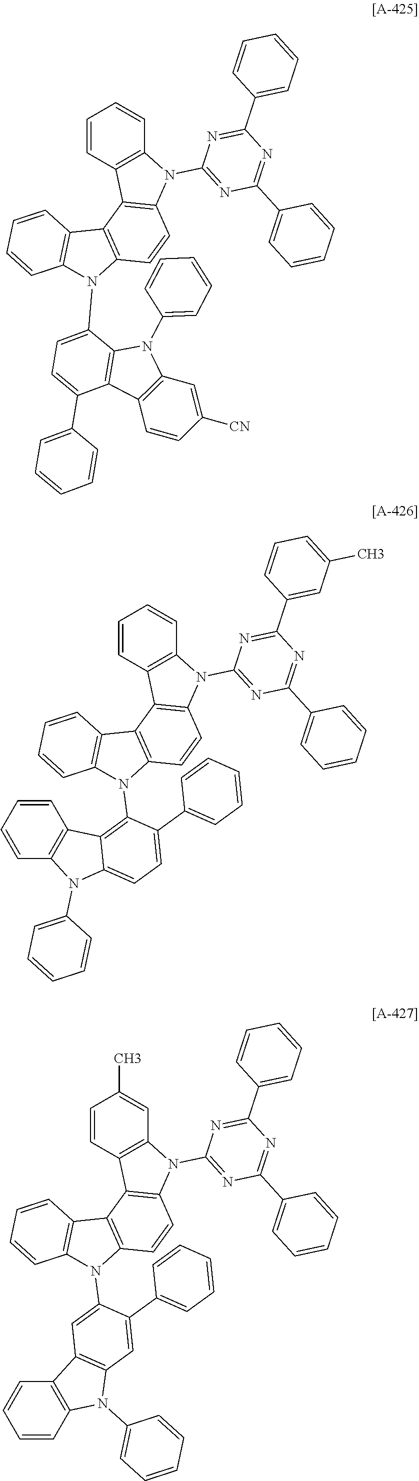 [SMILES: N#Cc1ccc2c3c(-c4ccccc4)ccc(-n4c5ccccc5c5c6c7ccccc7n(-c7nc(-c8ccccc8)nc(-c8ccccc8)n7)c6ccc54)c3n(-c3ccccc3)c2c1 ; Cc1cccc(-c2nc(-c3ccccc3)nc(-n3c4ccccc4c4c5c6ccccc6n(-c6c(-c7ccccc7)ccc7c6c6ccccc6n7-c6ccccc6)c5ccc43)n2)c1 ; Cc1ccc2c3c4c5ccccc5n(-c5cc6c7ccccc7n(-c7ccccc7)c6cc5-c5ccccc5)c4ccc3n(-c3nc(-c4ccccc4)nc(-c4ccccc4)n3)c2c1]

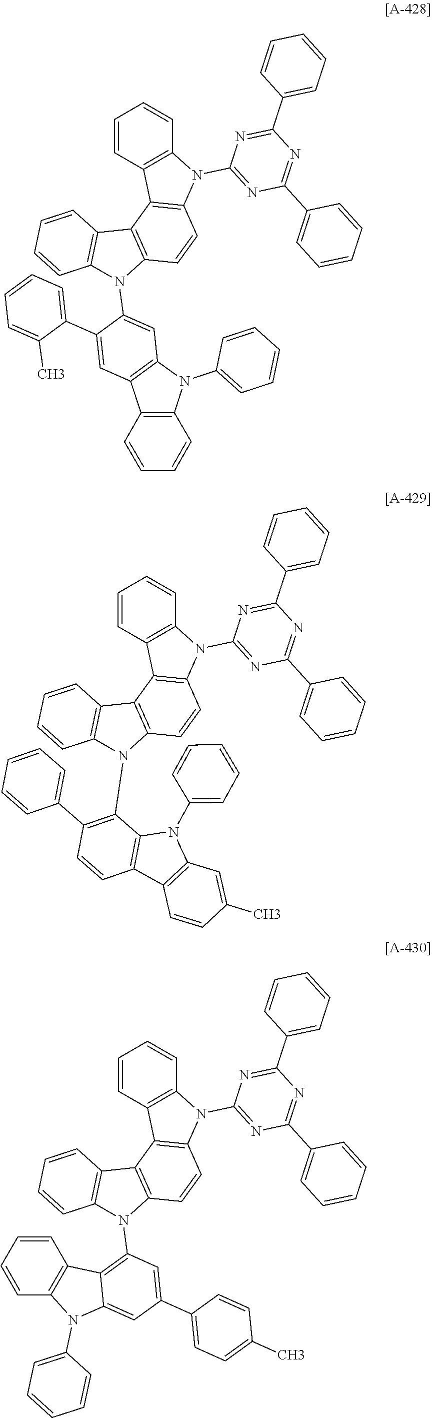 [SMILES: Cc1ccccc1-c1cc2c3ccccc3n(-c3ccccc3)c2cc1-n1c2ccccc2c2c3c4ccccc4n(-c4nc(-c5ccccc5)nc(-c5ccccc5)n4)c3ccc21 ; Cc1ccc2c3ccc(-c4ccccc4)c(-n4c5ccccc5c5c6c7ccccc7n(-c7nc(-c8ccccc8)nc(-c8ccccc8)n7)c6ccc54)c3n(-c3ccccc3)c2c1 ; Cc1ccc(-c2cc(-n3c4ccccc4c4c5c6ccccc6n(-c6nc(-c7ccccc7)nc(-c7ccccc7)n6)c5ccc43)c3c4ccccc4n(-c4ccccc4)c3c2)cc1]

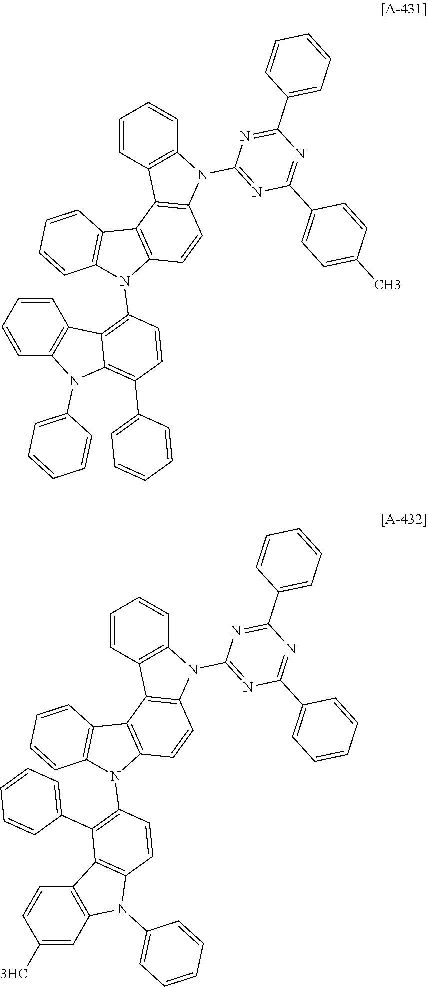

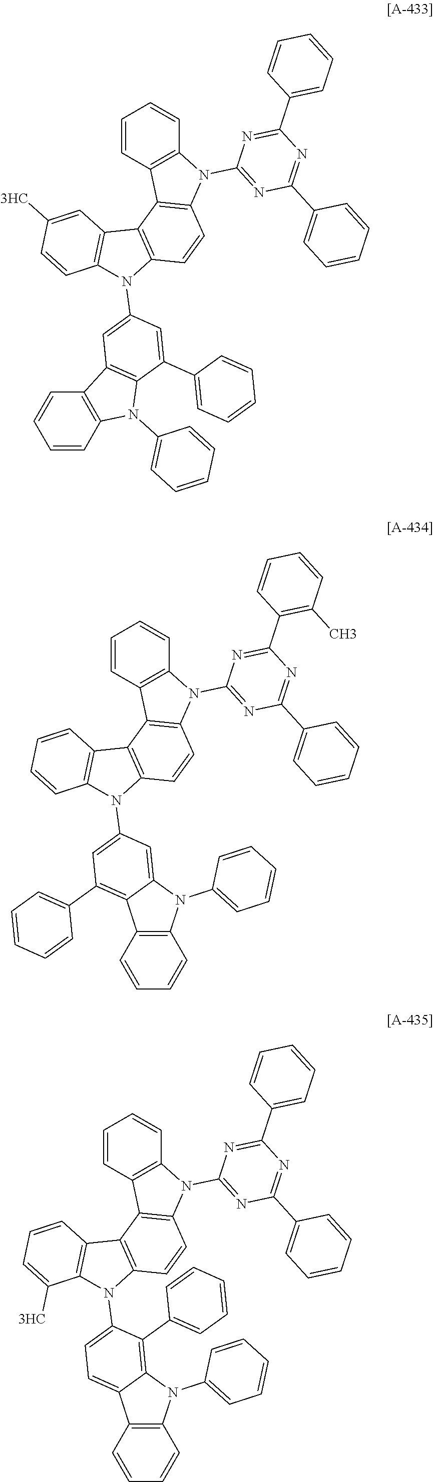

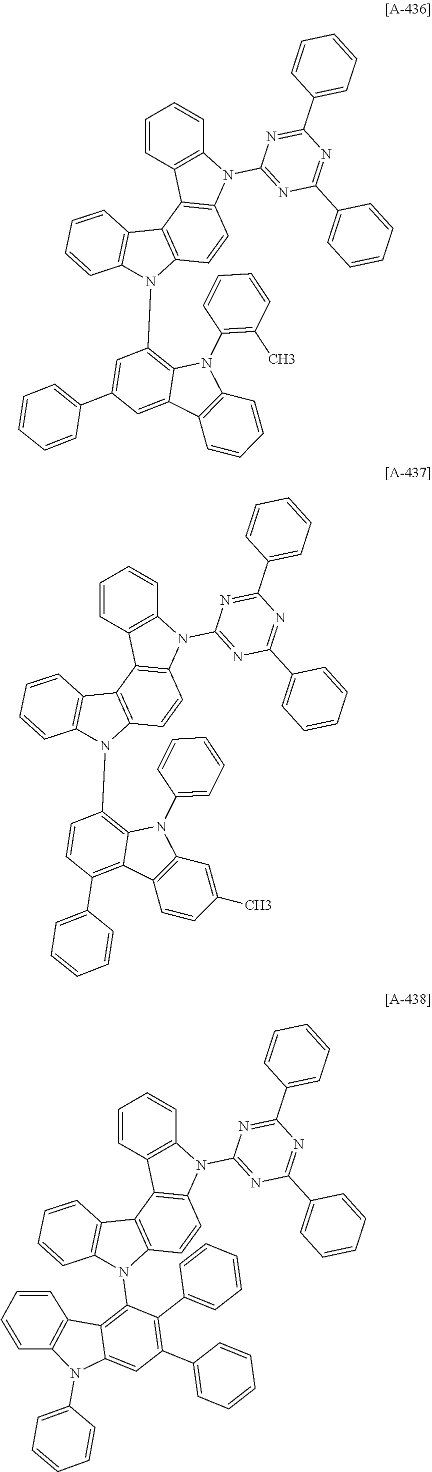 [SMILES: Cc1ccccc1-n1c2ccccc2c2cc(-c3ccccc3)cc(-n3c4ccccc4c4c5c6ccccc6n(-c6nc(-c7ccccc7)nc(-c7ccccc7)n6)c5ccc43)c21 ; Cc1ccc2c3c(-c4ccccc4)ccc(-n4c5ccccc5c5c6c7ccccc7n(-c7nc(-c8ccccc8)nc(-c8ccccc8)n7)c6ccc54)c3n(-c3ccccc3)c2c1 ; c1ccc(-c2nc(-c3ccccc3)nc(-n3c4ccccc4c4c5c6ccccc6n(-c6c(-c7ccccc7)c(-c7ccccc7)cc7c6c6ccccc6n7-c6ccccc6)c5ccc43)n2)cc1]

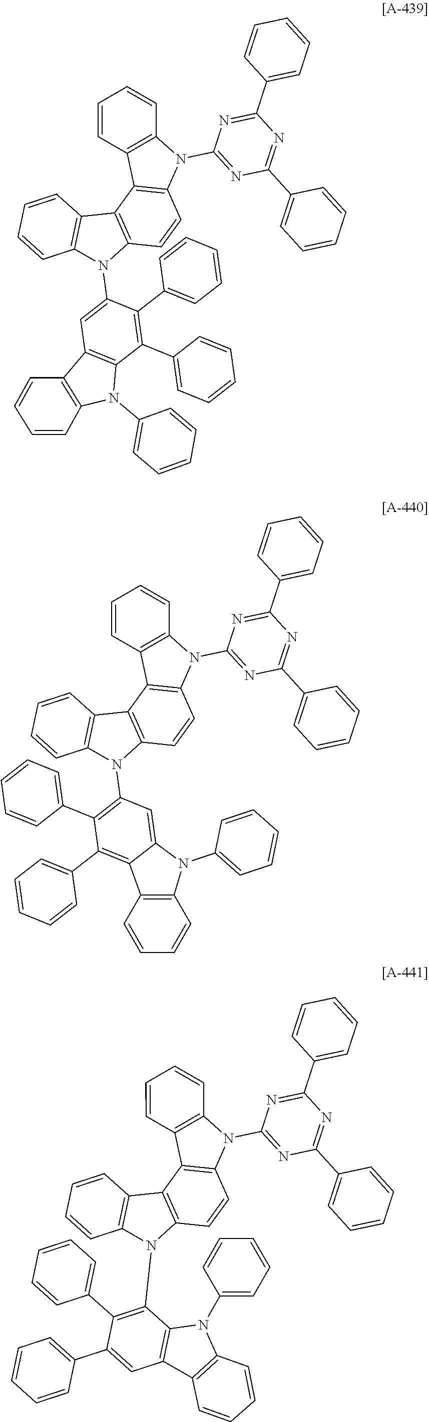 [SMILES: c1ccc(-c2nc(-c3ccccc3)nc(-n3c4ccccc4c4c5c6ccccc6n(-c6cc7c8ccccc8n(-c8ccccc8)c7c(-c7ccccc7)c6-c6ccccc6)c5ccc43)n2)cc1 ; c1ccc(-c2nc(-c3ccccc3)nc(-n3c4ccccc4c4c5c6ccccc6n(-c6cc7c(c(-c8ccccc8)c6-c6ccccc6)c6ccccc6n7-c6ccccc6)c5ccc43)n2)cc1 ; c1ccc(-c2nc(-c3ccccc3)nc(-n3c4ccccc4c4c5c6ccccc6n(-c6c(-c7ccccc7)c(-c7ccccc7)cc7c8ccccc8n(-c8ccccc8)c67)c5ccc43)n2)cc1]

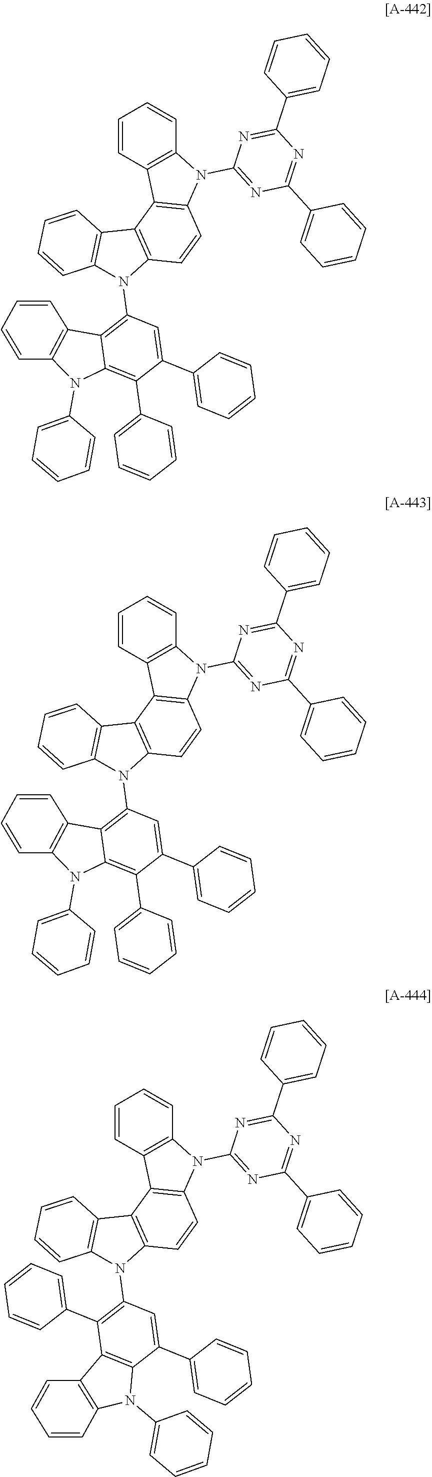 [SMILES: c1ccc(-c2nc(-c3ccccc3)nc(-n3c4ccccc4c4c5c6ccccc6n(-c6cc(-c7ccccc7)c(-c7ccccc7)c7c6c6ccccc6n7-c6ccccc6)c5ccc43)n2)cc1 ; c1ccc(-c2nc(-c3ccccc3)nc(-n3c4ccccc4c4c5c6ccccc6n(-c6cc(-c7ccccc7)c(-c7ccccc7)c7c6c6ccccc6n7-c6ccccc6)c5ccc43)n2)cc1 ; c1ccc(-c2nc(-c3ccccc3)nc(-n3c4ccccc4c4c5c6ccccc6n(-c6cc(-c7ccccc7)c7c(c6-c6ccccc6)c6ccccc6n7-c6ccccc6)c5ccc43)n2)cc1]

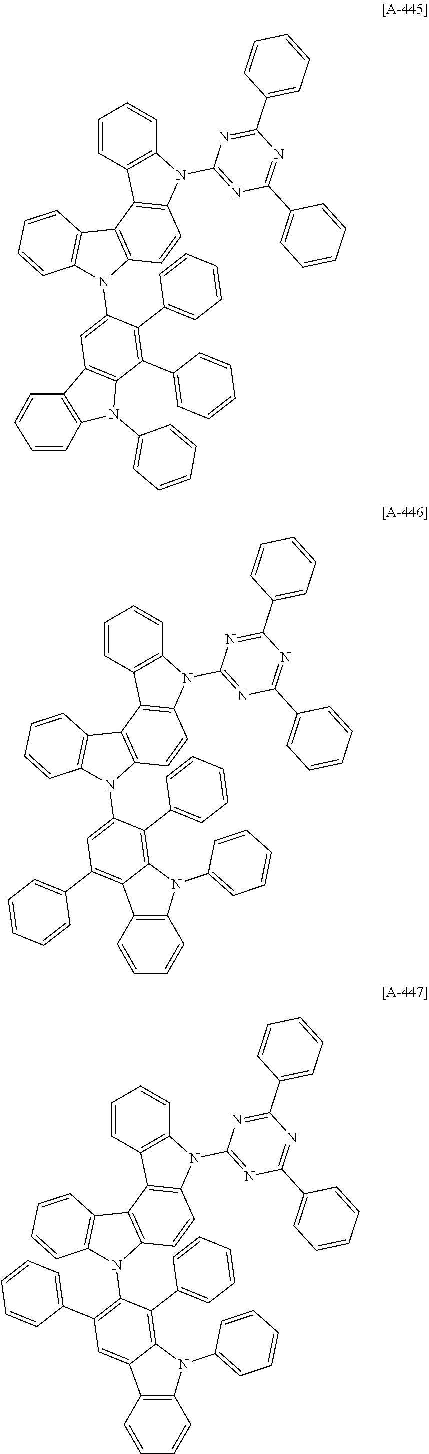 [SMILES: c1ccc(-c2nc(-c3ccccc3)nc(-n3c4ccccc4c4c5c6ccccc6n(-c6cc7c8ccccc8n(-c8ccccc8)c7c(-c7ccccc7)c6-c6ccccc6)c5ccc43)n2)cc1 ; c1ccc(-c2nc(-c3ccccc3)nc(-n3c4ccccc4c4c5c6ccccc6n(-c6cc(-c7ccccc7)c7c8ccccc8n(-c8ccccc8)c7c6-c6ccccc6)c5ccc43)n2)cc1 ; c1ccc(-c2nc(-c3ccccc3)nc(-n3c4ccccc4c4c5c6ccccc6n(-c6c(-c7ccccc7)cc7c8ccccc8n(-c8ccccc8)c7c6-c6ccccc6)c5ccc43)n2)cc1]

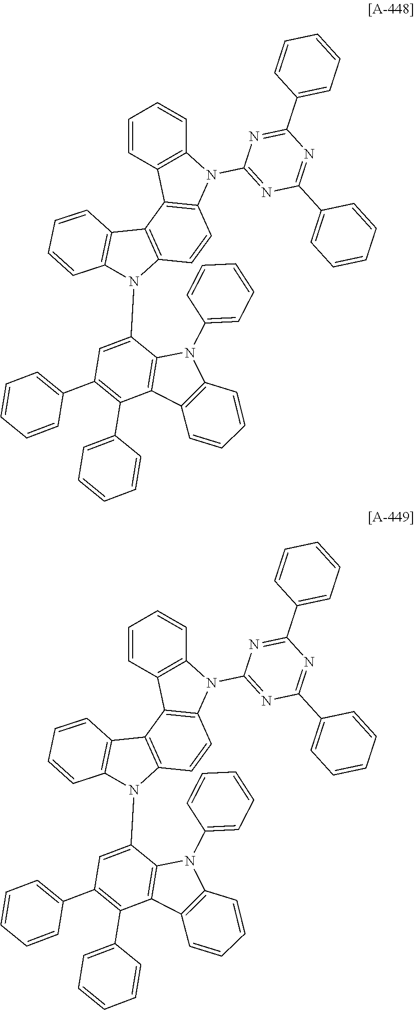 [SMILES: c1ccc(-c2nc(-c3ccccc3)nc(-n3c4ccccc4c4c5c6ccccc6n(-c6cc(-c7ccccc7)c(-c7ccccc7)c7c8ccccc8n(-c8ccccc8)c67)c5ccc43)n2)cc1 ; c1ccc(-c2nc(-c3ccccc3)nc(-n3c4ccccc4c4c5c6ccccc6n(-c6cc(-c7ccccc7)c(-c7ccccc7)c7c8ccccc8n(-c8ccccc8)c67)c5ccc43)n2)cc1]

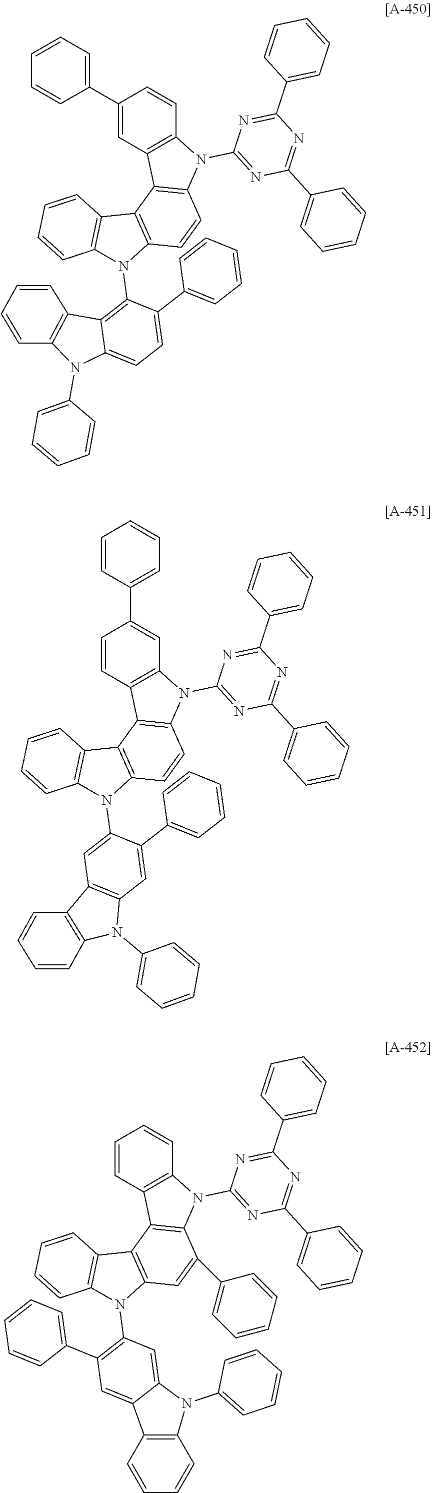 [SMILES: c1ccc(-c2ccc3c(c2)c2c4c5ccccc5n(-c5c(-c6ccccc6)ccc6c5c5ccccc5n6-c5ccccc5)c4ccc2n3-c2nc(-c3ccccc3)nc(-c3ccccc3)n2)cc1 ; c1ccc(-c2ccc3c4c5c6ccccc6n(-c6cc7c8ccccc8n(-c8ccccc8)c7cc6-c6ccccc6)c5ccc4n(-c4nc(-c5ccccc5)nc(-c5ccccc5)n4)c3c2)cc1 ; c1ccc(-c2nc(-c3ccccc3)nc(-n3c4ccccc4c4c5c6ccccc6n(-c6cc7c(cc6-c6ccccc6)c6ccccc6n7-c6ccccc6)c5cc(-c5ccccc5)c43)n2)cc1]

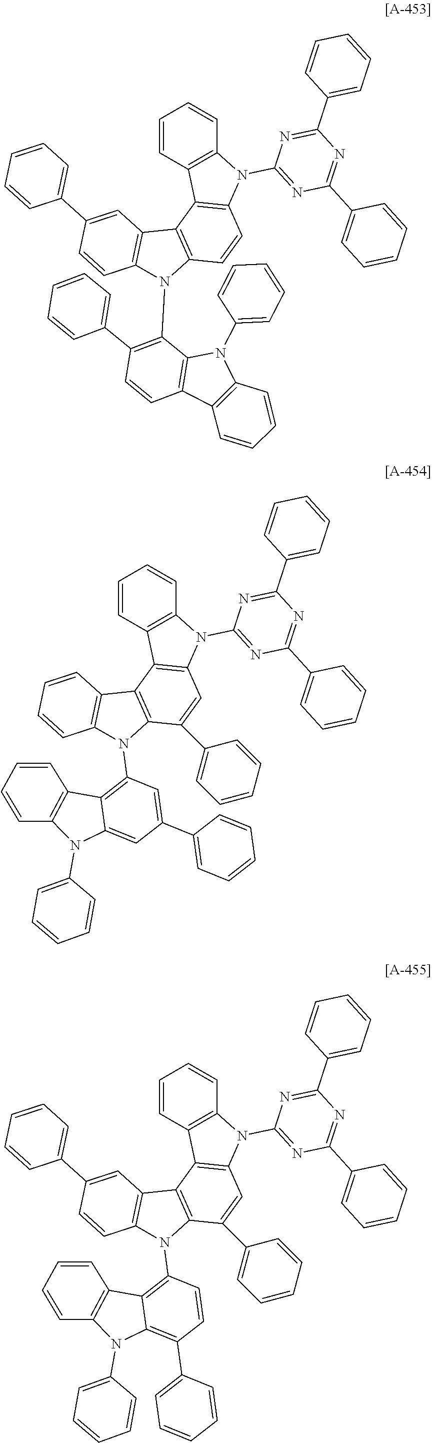 [SMILES: c1ccc(-c2ccc3c(c2)c2c4c5ccccc5n(-c5nc(-c6ccccc6)nc(-c6ccccc6)n5)c4ccc2n3-c2c(-c3ccccc3)ccc3c4ccccc4n(-c4ccccc4)c23)cc1 ; c1ccc(-c2cc(-n3c4ccccc4c4c5c6ccccc6n(-c6nc(-c7ccccc7)nc(-c7ccccc7)n6)c5cc(-c5ccccc5)c43)c3c4ccccc4n(-c4ccccc4)c3c2)cc1 ; c1ccc(-c2ccc3c(c2)c2c4c5ccccc5n(-c5nc(-c6ccccc6)nc(-c6ccccc6)n5)c4cc(-c4ccccc4)c2n3-c2ccc(-c3ccccc3)c3c2c2ccccc2n3-c2ccccc2)cc1]

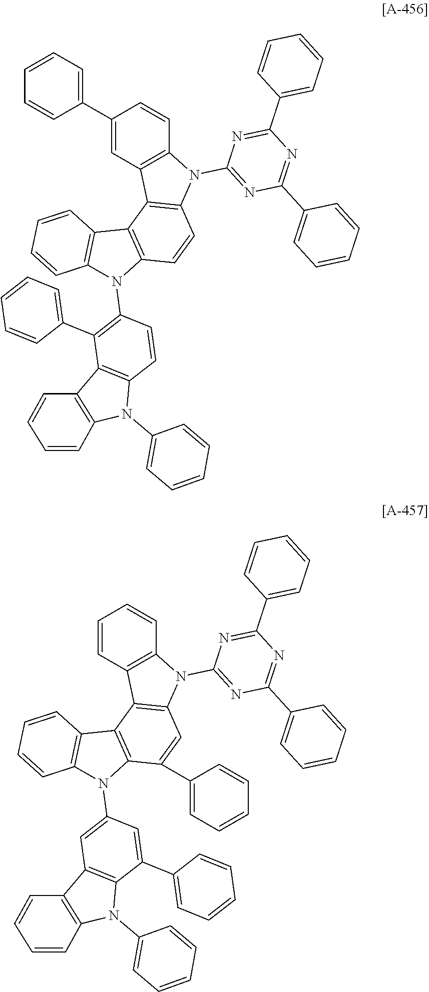 [SMILES: c1ccc(-c2ccc3c(c2)c2c4c5ccccc5n(-c5ccc6c(c5-c5ccccc5)c5ccccc5n6-c5ccccc5)c4ccc2n3-c2nc(-c3ccccc3)nc(-c3ccccc3)n2)cc1 ; c1ccc(-c2nc(-c3ccccc3)nc(-n3c4ccccc4c4c5c6ccccc6n(-c6cc(-c7ccccc7)c7c(c6)c6ccccc6n7-c6ccccc6)c5c(-c5ccccc5)cc43)n2)cc1]

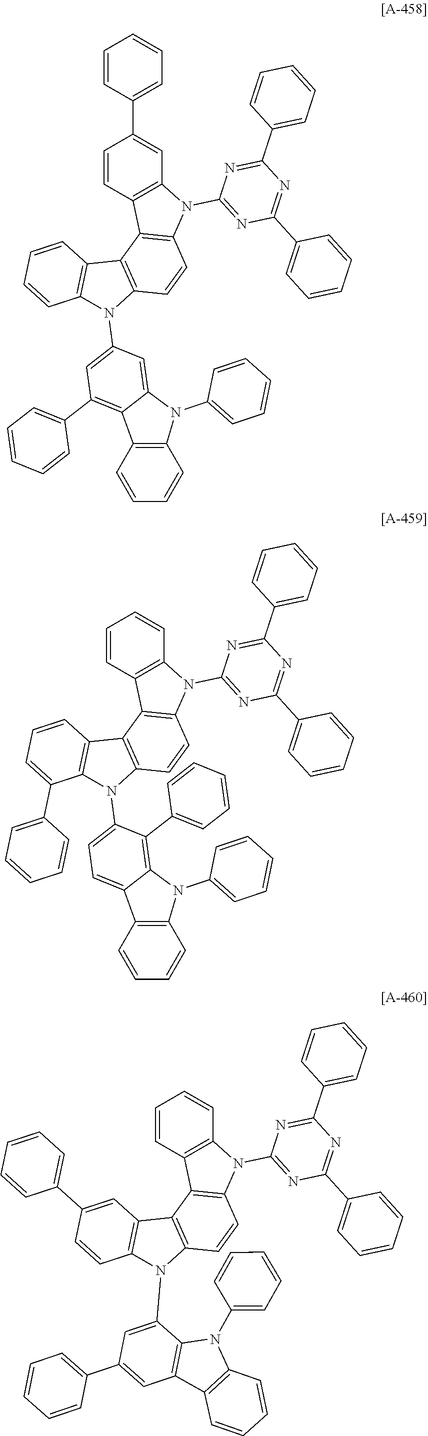 [SMILES: c1ccc(-c2ccc3c4c5c6ccccc6n(-c6cc(-c7ccccc7)c7c8ccccc8n(-c8ccccc8)c7c6)c5ccc4n(-c4nc(-c5ccccc5)nc(-c5ccccc5)n4)c3c2)cc1 ; c1ccc(-c2nc(-c3ccccc3)nc(-n3c4ccccc4c4c5c6cccc(-c7ccccc7)c6n(-c6ccc7c8ccccc8n(-c8ccccc8)c7c6-c6ccccc6)c5ccc43)n2)cc1 ; c1ccc(-c2ccc3c(c2)c2c4c5ccccc5n(-c5nc(-c6ccccc6)nc(-c6ccccc6)n5)c4ccc2n3-c2cc(-c3ccccc3)cc3c4ccccc4n(-c4ccccc4)c23)cc1]

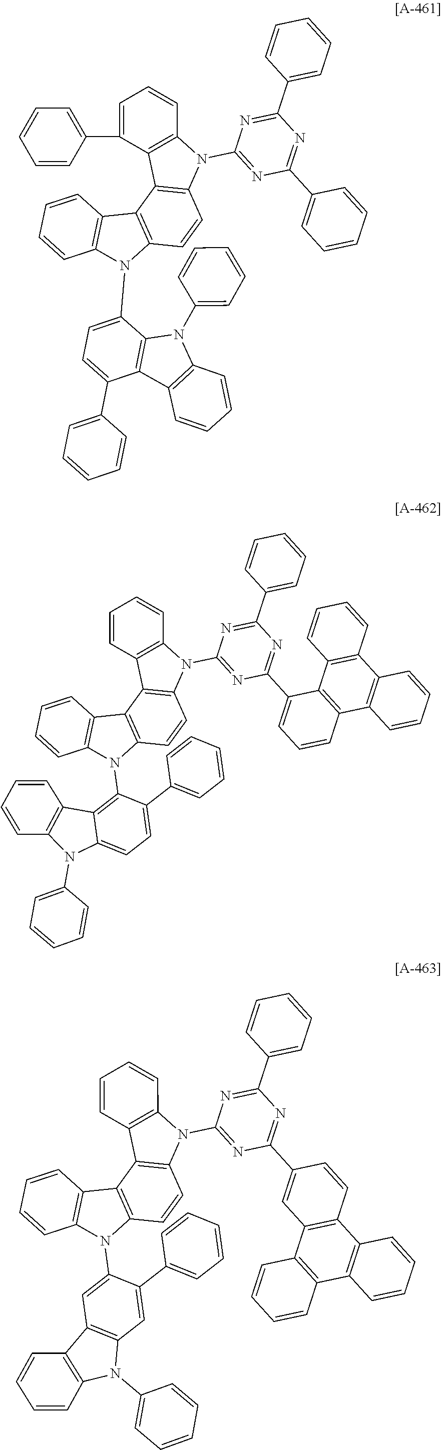 [SMILES: c1ccc(-c2nc(-c3ccccc3)nc(-n3c4cccc(-c5ccccc5)c4c4c5c6ccccc6n(-c6ccc(-c7ccccc7)c7c8ccccc8n(-c8ccccc8)c67)c5ccc43)n2)cc1 ; c1ccc(-c2nc(-c3cccc4c5ccccc5c5ccccc5c34)nc(-n3c4ccccc4c4c5c6ccccc6n(-c6c(-c7ccccc7)ccc7c6c6ccccc6n7-c6ccccc6)c5ccc43)n2)cc1 ; c1ccc(-c2nc(-c3ccc4c5ccccc5c5ccccc5c4c3)nc(-n3c4ccccc4c4c5c6ccccc6n(-c6cc7c8ccccc8n(-c8ccccc8)c7cc6-c6ccccc6)c5ccc43)n2)cc1]

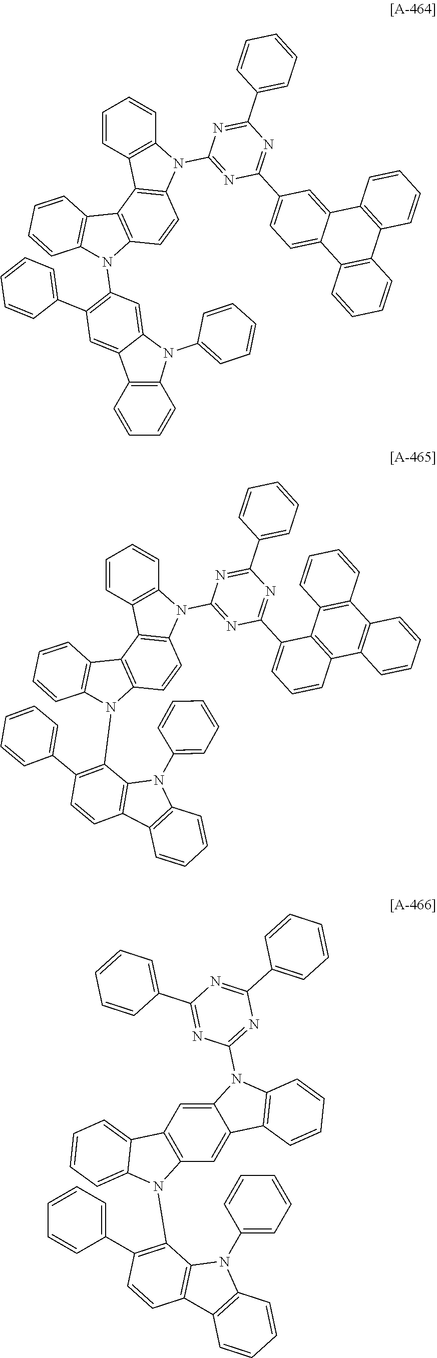 [SMILES: c1ccc(-c2nc(-c3ccc4c5ccccc5c5ccccc5c4c3)nc(-n3c4ccccc4c4c5c6ccccc6n(-c6cc7c(cc6-c6ccccc6)c6ccccc6n7-c6ccccc6)c5ccc43)n2)cc1 ; c1ccc(-c2nc(-c3cccc4c5ccccc5c5ccccc5c34)nc(-n3c4ccccc4c4c5c6ccccc6n(-c6c(-c7ccccc7)ccc7c8ccccc8n(-c8ccccc8)c67)c5ccc43)n2)cc1 ; c1ccc(-c2nc(-c3ccccc3)nc(-n3c4ccccc4c4cc5c(cc43)c3ccccc3n5-c3c(-c4ccccc4)ccc4c5ccccc5n(-c5ccccc5)c34)n2)cc1]

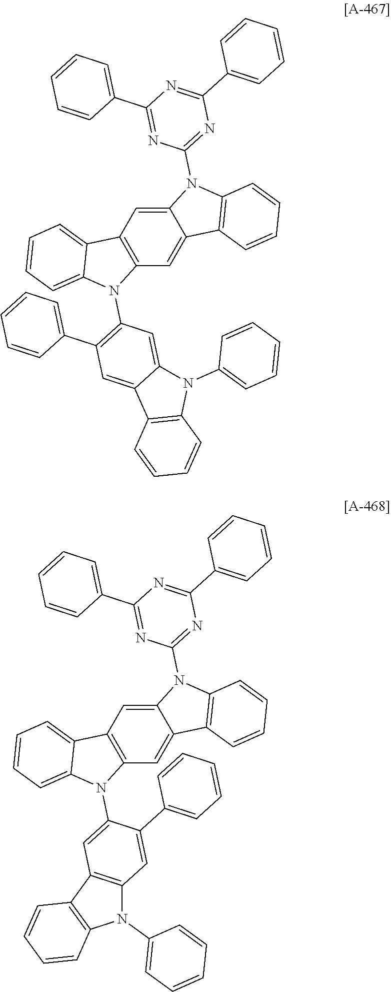 [SMILES: c1ccc(-c2nc(-c3ccccc3)nc(-n3c4ccccc4c4cc5c(cc43)c3ccccc3n5-c3cc4c(cc3-c3ccccc3)c3ccccc3n4-c3ccccc3)n2)cc1 ; c1ccc(-c2nc(-c3ccccc3)nc(-n3c4ccccc4c4cc5c(cc43)c3ccccc3n5-c3cc4c5ccccc5n(-c5ccccc5)c4cc3-c3ccccc3)n2)cc1]

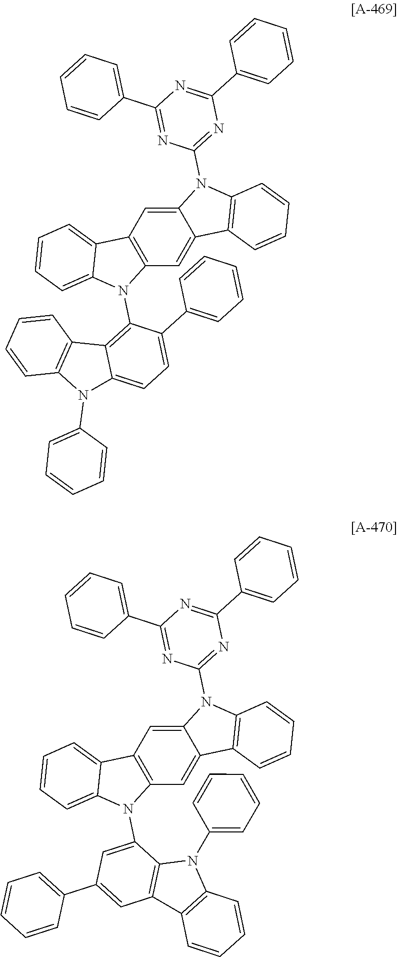 [SMILES: c1ccc(-c2nc(-c3ccccc3)nc(-n3c4ccccc4c4cc5c(cc43)c3ccccc3n5-c3c(-c4ccccc4)ccc4c3c3ccccc3n4-c3ccccc3)n2)cc1 ; c1ccc(-c2cc(-n3c4ccccc4c4cc5c(cc43)c3ccccc3n5-c3nc(-c4ccccc4)nc(-c4ccccc4)n3)c3c(c2)c2ccccc2n3-c2ccccc2)cc1]

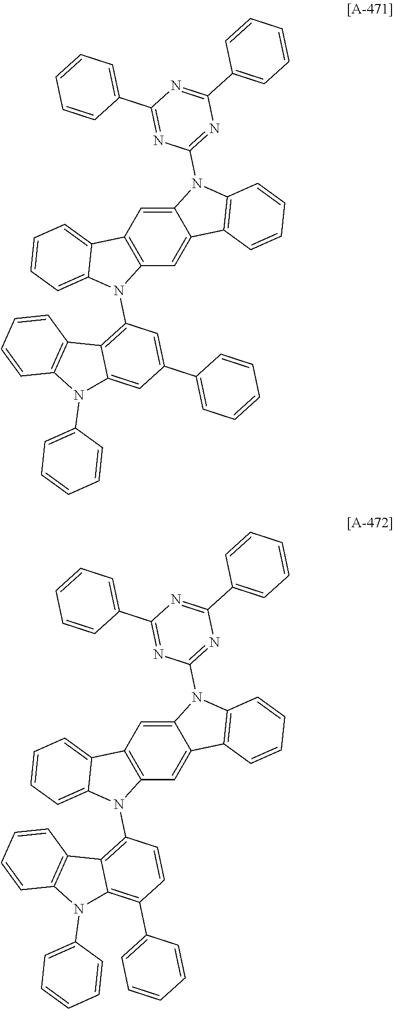 [SMILES: c1ccc(-c2cc(-n3c4ccccc4c4cc5c(cc43)c3ccccc3n5-c3nc(-c4ccccc4)nc(-c4ccccc4)n3)c3c4ccccc4n(-c4ccccc4)c3c2)cc1 ; c1ccc(-c2nc(-c3ccccc3)nc(-n3c4ccccc4c4cc5c(cc43)c3ccccc3n5-c3ccc(-c4ccccc4)c4c3c3ccccc3n4-c3ccccc3)n2)cc1]

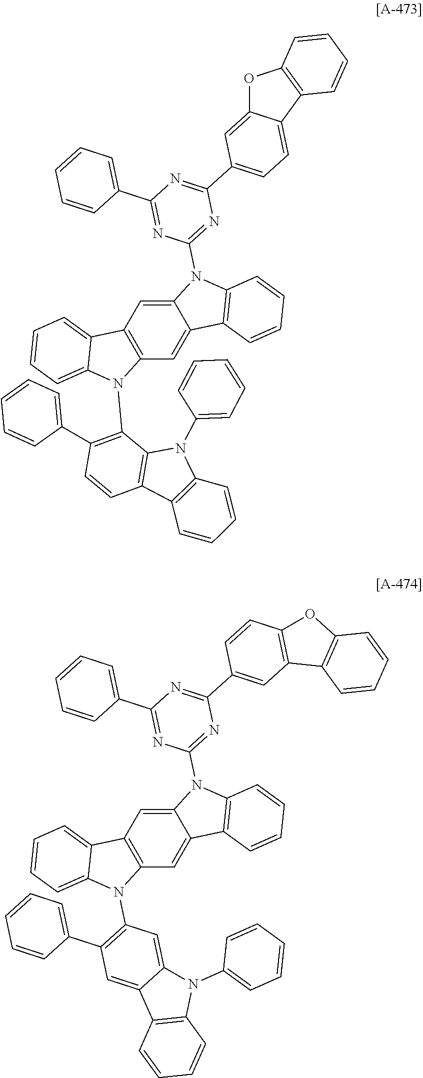 [SMILES: c1ccc(-c2nc(-c3ccc4c(c3)oc3ccccc34)nc(-n3c4ccccc4c4cc5c(cc43)c3ccccc3n5-c3c(-c4ccccc4)ccc4c5ccccc5n(-c5ccccc5)c34)n2)cc1 ; c1ccc(-c2nc(-c3ccc4oc5ccccc5c4c3)nc(-n3c4ccccc4c4cc5c(cc43)c3ccccc3n5-c3cc4c(cc3-c3ccccc3)c3ccccc3n4-c3ccccc3)n2)cc1]

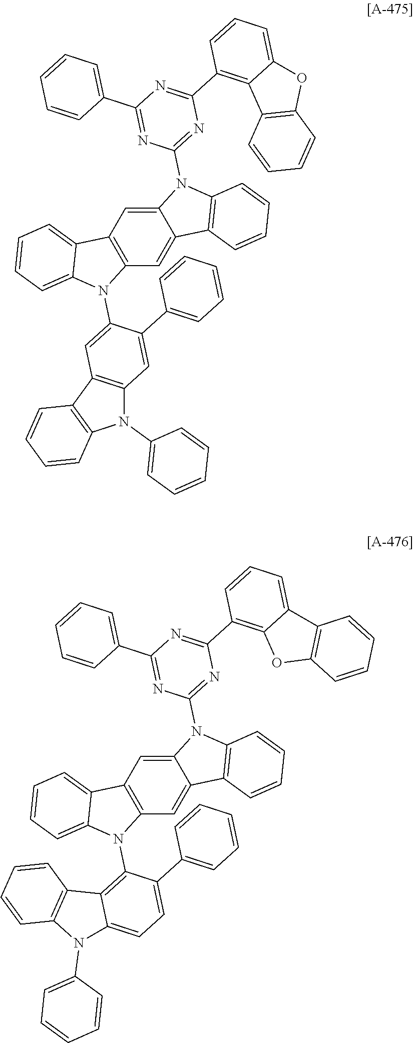 [SMILES: c1ccc(-c2nc(-c3cccc4oc5ccccc5c34)nc(-n3c4ccccc4c4cc5c(cc43)c3ccccc3n5-c3cc4c5ccccc5n(-c5ccccc5)c4cc3-c3ccccc3)n2)cc1 ; c1ccc(-c2nc(-c3cccc4c3oc3ccccc34)nc(-n3c4ccccc4c4cc5c(cc43)c3ccccc3n5-c3c(-c4ccccc4)ccc4c3c3ccccc3n4-c3ccccc3)n2)cc1]

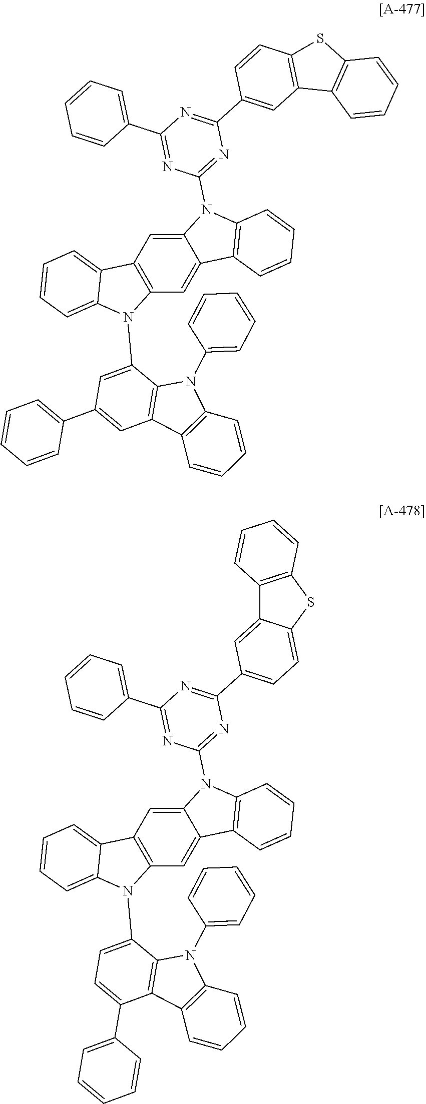 [SMILES: c1ccc(-c2cc(-n3c4ccccc4c4cc5c(cc43)c3ccccc3n5-c3nc(-c4ccccc4)nc(-c4ccc5sc6ccccc6c5c4)n3)c3c(c2)c2ccccc2n3-c2ccccc2)cc1 ; c1ccc(-c2nc(-c3ccc4sc5ccccc5c4c3)nc(-n3c4ccccc4c4cc5c(cc43)c3ccccc3n5-c3ccc(-c4ccccc4)c4c5ccccc5n(-c5ccccc5)c34)n2)cc1]

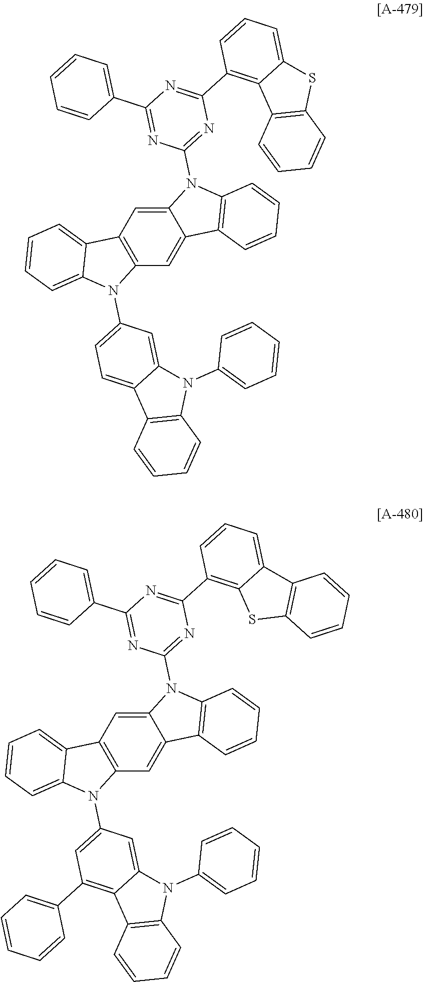 [SMILES: c1ccc(-c2nc(-c3cccc4sc5ccccc5c34)nc(-n3c4ccccc4c4cc5c(cc43)c3ccccc3n5-c3ccc4c5ccccc5n(-c5ccccc5)c4c3)n2)cc1 ; c1ccc(-c2nc(-c3cccc4c3sc3ccccc34)nc(-n3c4ccccc4c4cc5c(cc43)c3ccccc3n5-c3cc(-c4ccccc4)c4c5ccccc5n(-c5ccccc5)c4c3)n2)cc1]

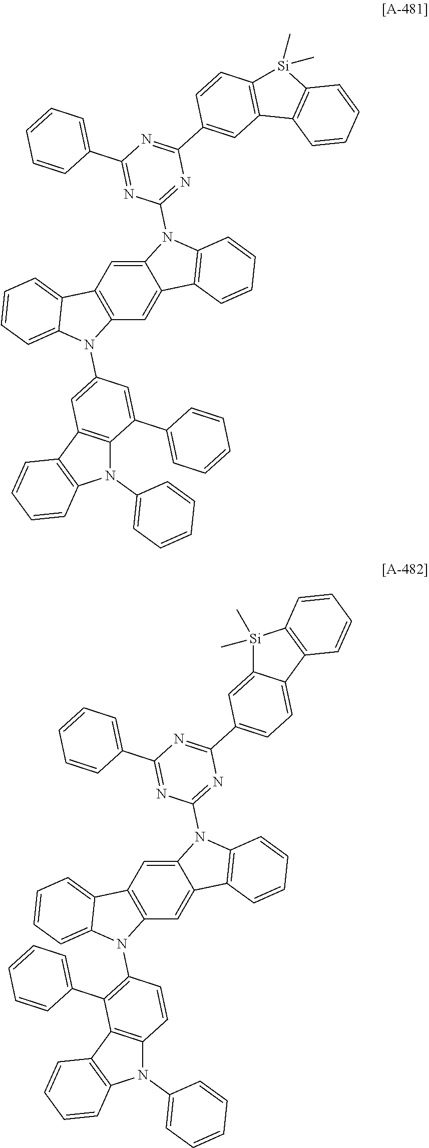 [SMILES: C[Si]1(C)c2ccccc2-c2cc(-c3nc(-c4ccccc4)nc(-n4c5ccccc5c5cc6c(cc54)c4ccccc4n6-c4cc(-c5ccccc5)c5c(c4)c4ccccc4n5-c4ccccc4)n3)ccc21 ; C[Si]1(C)c2ccccc2-c2ccc(-c3nc(-c4ccccc4)nc(-n4c5ccccc5c5cc6c(cc54)c4ccccc4n6-c4ccc5c(c4-c4ccccc4)c4ccccc4n5-c4ccccc4)n3)cc21]

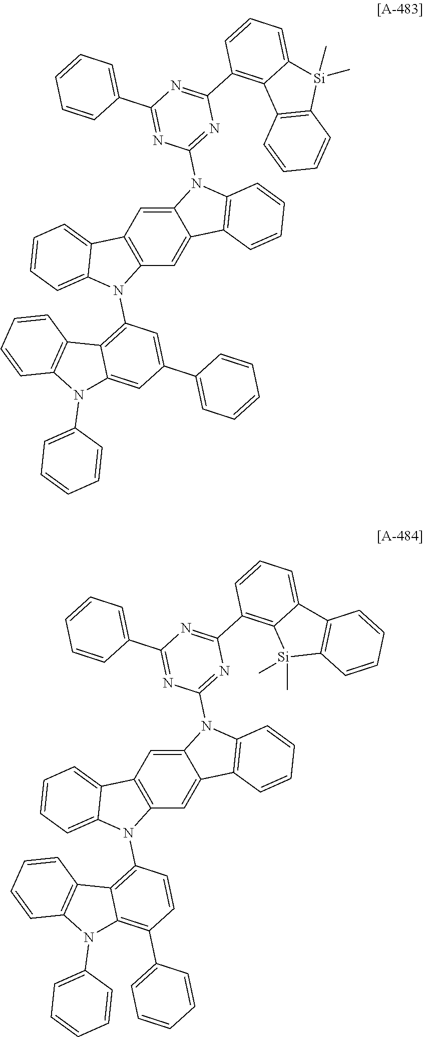 [SMILES: C[Si]1(C)c2ccccc2-c2c(-c3nc(-c4ccccc4)nc(-n4c5ccccc5c5cc6c(cc54)c4ccccc4n6-c4cc(-c5ccccc5)cc5c4c4ccccc4n5-c4ccccc4)n3)cccc21 ; C[Si]1(C)c2ccccc2-c2cccc(-c3nc(-c4ccccc4)nc(-n4c5ccccc5c5cc6c(cc54)c4ccccc4n6-c4ccc(-c5ccccc5)c5c4c4ccccc4n5-c4ccccc4)n3)c21]

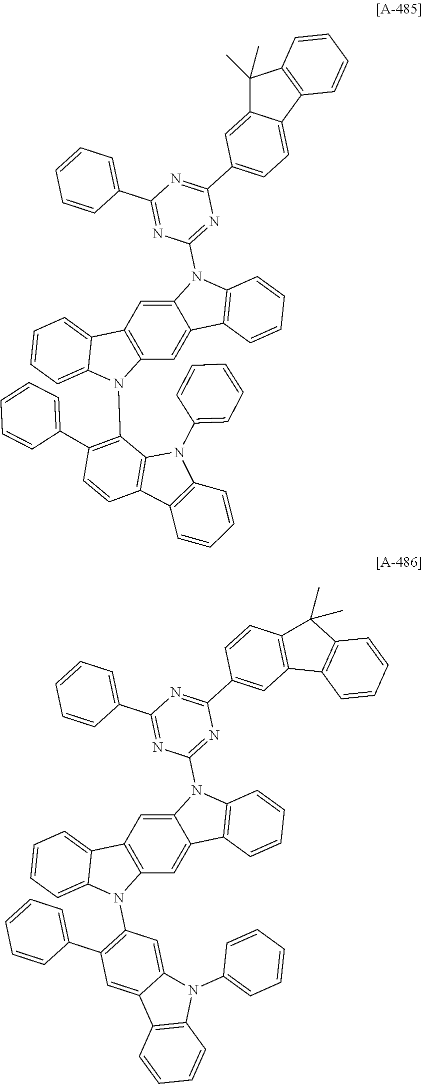 [SMILES: CC1(C)c2ccccc2-c2ccc(-c3nc(-c4ccccc4)nc(-n4c5ccccc5c5cc6c(cc54)c4ccccc4n6-c4c(-c5ccccc5)ccc5c6ccccc6n(-c6ccccc6)c45)n3)cc21 ; CC1(C)c2ccccc2-c2cc(-c3nc(-c4ccccc4)nc(-n4c5ccccc5c5cc6c(cc54)c4ccccc4n6-c4cc5c(cc4-c4ccccc4)c4ccccc4n5-c4ccccc4)n3)ccc21]

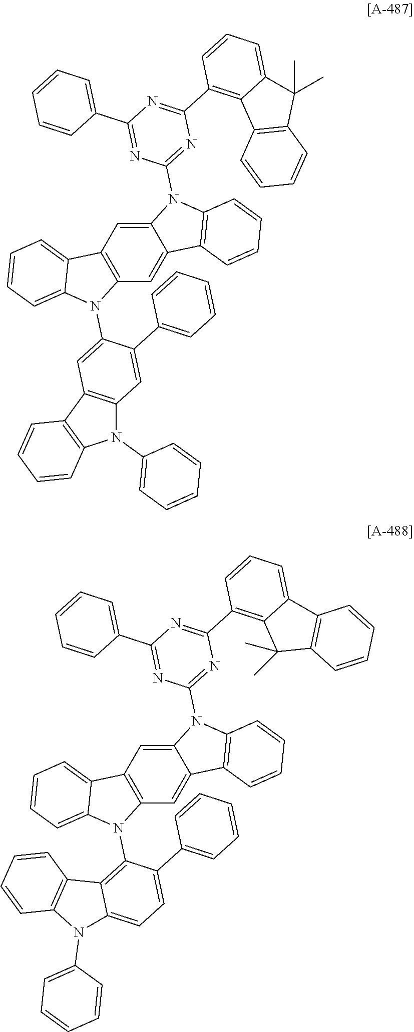 [SMILES: CC1(C)c2ccccc2-c2c(-c3nc(-c4ccccc4)nc(-n4c5ccccc5c5cc6c(cc54)c4ccccc4n6-c4cc5c6ccccc6n(-c6ccccc6)c5cc4-c4ccccc4)n3)cccc21 ; CC1(C)c2ccccc2-c2cccc(-c3nc(-c4ccccc4)nc(-n4c5ccccc5c5cc6c(cc54)c4ccccc4n6-c4c(-c5ccccc5)ccc5c4c4ccccc4n5-c4ccccc4)n3)c21]

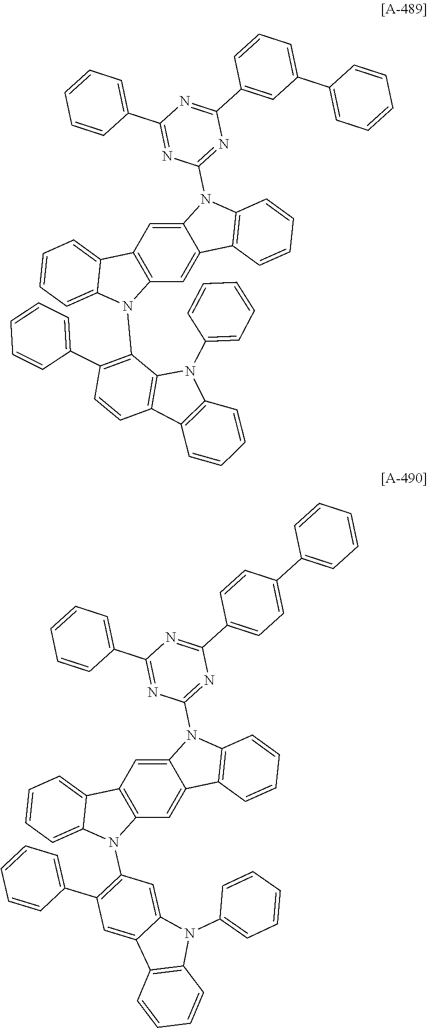 [SMILES: c1ccc(-c2cccc(-c3nc(-c4ccccc4)nc(-n4c5ccccc5c5cc6c(cc54)c4ccccc4n6-c4c(-c5ccccc5)ccc5c6ccccc6n(-c6ccccc6)c45)n3)c2)cc1 ; c1ccc(-c2ccc(-c3nc(-c4ccccc4)nc(-n4c5ccccc5c5cc6c(cc54)c4ccccc4n6-c4cc5c(cc4-c4ccccc4)c4ccccc4n5-c4ccccc4)n3)cc2)cc1]

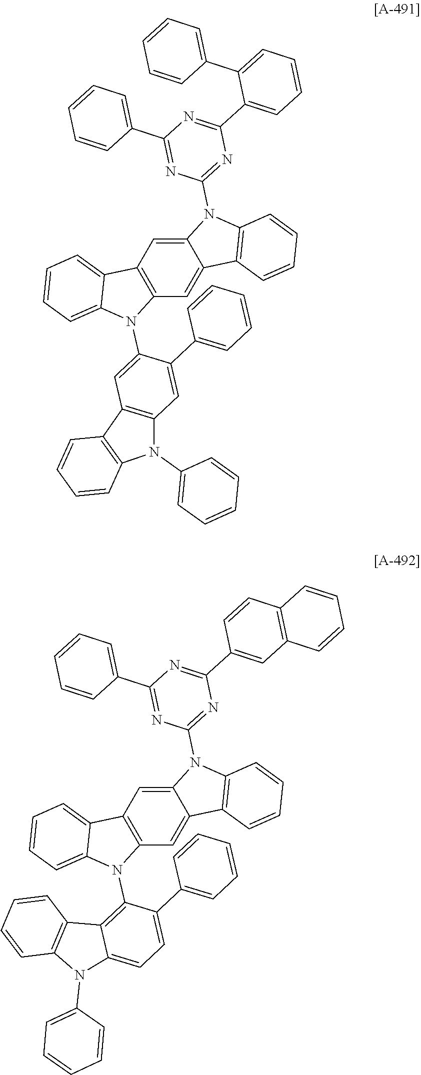 [SMILES: c1ccc(-c2nc(-c3ccccc3-c3ccccc3)nc(-n3c4ccccc4c4cc5c(cc43)c3ccccc3n5-c3cc4c5ccccc5n(-c5ccccc5)c4cc3-c3ccccc3)n2)cc1 ; c1ccc(-c2nc(-c3ccc4ccccc4c3)nc(-n3c4ccccc4c4cc5c(cc43)c3ccccc3n5-c3c(-c4ccccc4)ccc4c3c3ccccc3n4-c3ccccc3)n2)cc1]

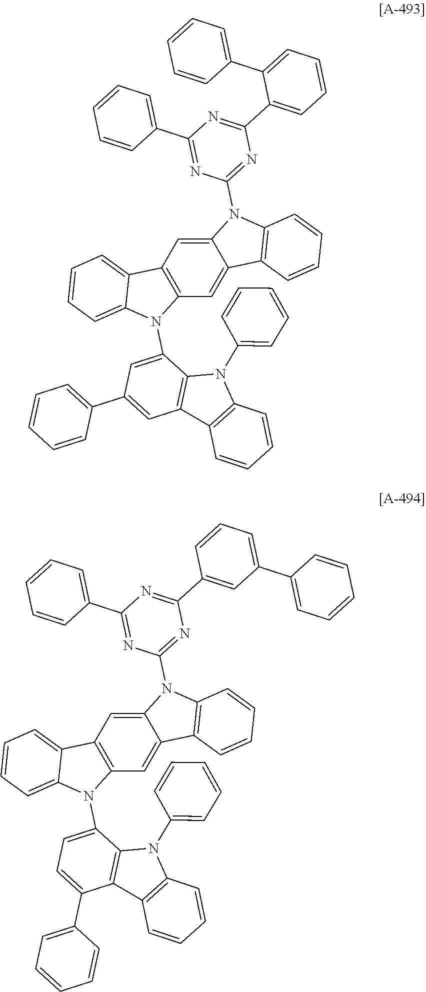 [SMILES: c1ccc(-c2cc(-n3c4ccccc4c4cc5c(cc43)c3ccccc3n5-c3nc(-c4ccccc4)nc(-c4ccccc4-c4ccccc4)n3)c3c(c2)c2ccccc2n3-c2ccccc2)cc1 ; c1ccc(-c2cccc(-c3nc(-c4ccccc4)nc(-n4c5ccccc5c5cc6c(cc54)c4ccccc4n6-c4ccc(-c5ccccc5)c5c6ccccc6n(-c6ccccc6)c45)n3)c2)cc1]

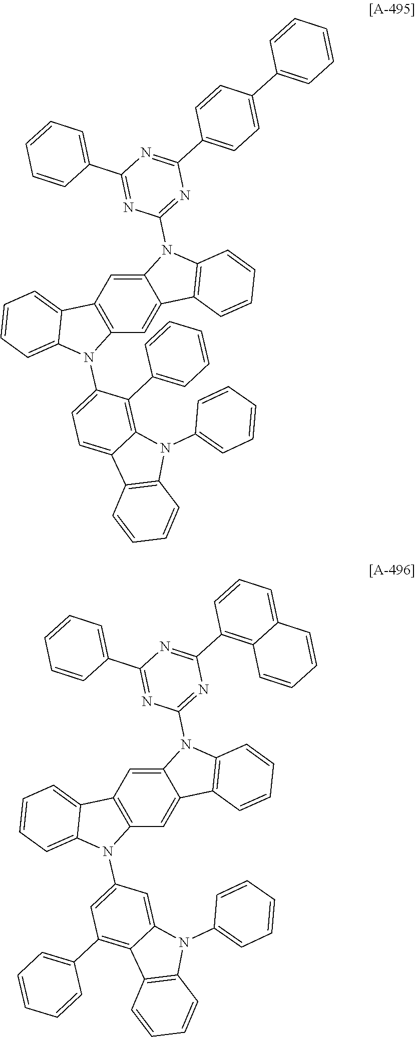 [SMILES: c1ccc(-c2ccc(-c3nc(-c4ccccc4)nc(-n4c5ccccc5c5cc6c(cc54)c4ccccc4n6-c4ccc5c6ccccc6n(-c6ccccc6)c5c4-c4ccccc4)n3)cc2)cc1 ; c1ccc(-c2nc(-c3cccc4ccccc34)nc(-n3c4ccccc4c4cc5c(cc43)c3ccccc3n5-c3cc(-c4ccccc4)c4c5ccccc5n(-c5ccccc5)c4c3)n2)cc1]

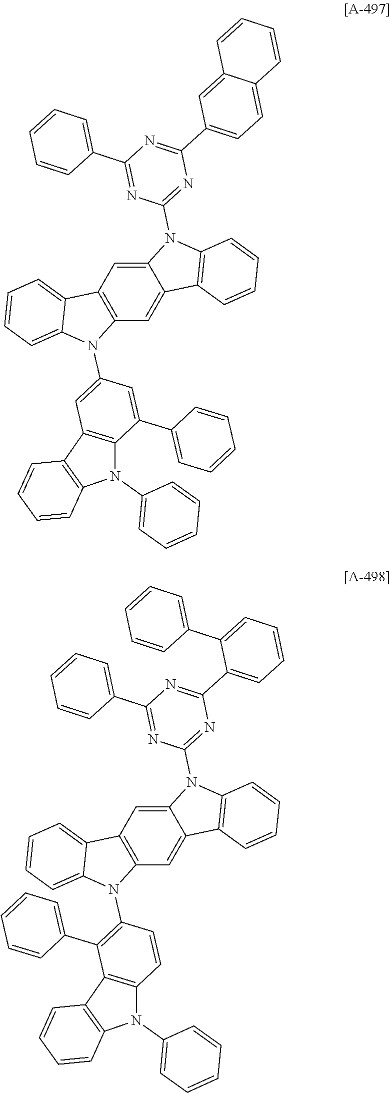

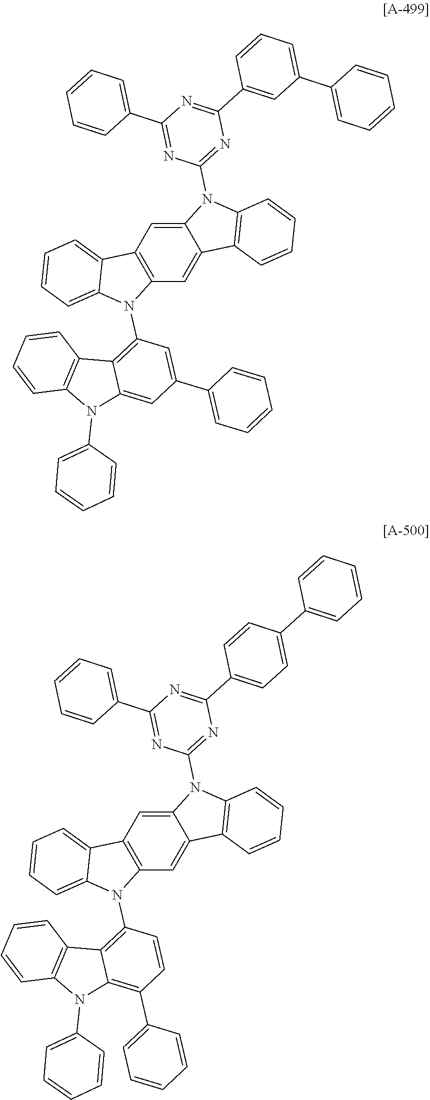 [SMILES: c1ccc(-c2cccc(-c3nc(-c4ccccc4)nc(-n4c5ccccc5c5cc6c(cc54)c4ccccc4n6-c4cc(-c5ccccc5)cc5c4c4ccccc4n5-c4ccccc4)n3)c2)cc1 ; c1ccc(-c2ccc(-c3nc(-c4ccccc4)nc(-n4c5ccccc5c5cc6c(cc54)c4ccccc4n6-c4ccc(-c5ccccc5)c5c4c4ccccc4n5-c4ccccc4)n3)cc2)cc1]

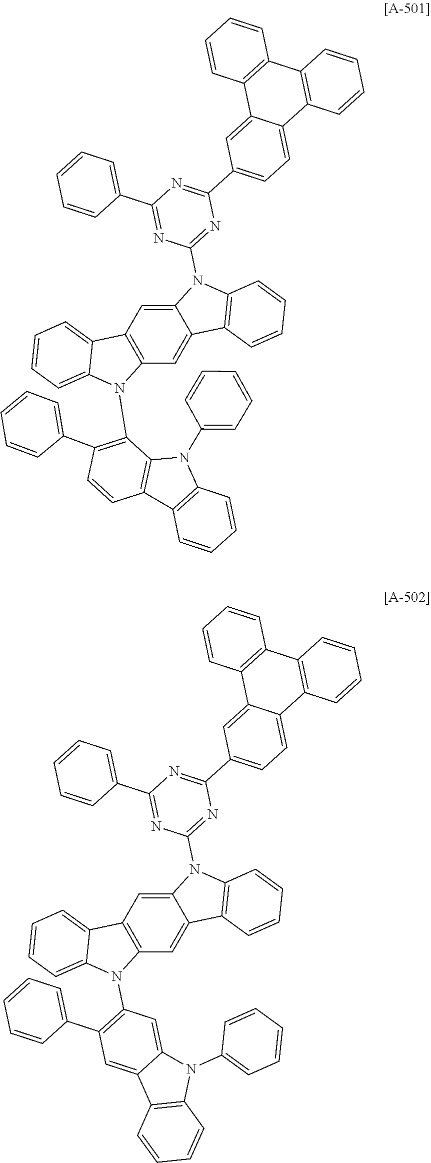 [SMILES: c1ccc(-c2nc(-c3ccc4c5ccccc5c5ccccc5c4c3)nc(-n3c4ccccc4c4cc5c(cc43)c3ccccc3n5-c3c(-c4ccccc4)ccc4c5ccccc5n(-c5ccccc5)c34)n2)cc1 ; c1ccc(-c2nc(-c3ccc4c5ccccc5c5ccccc5c4c3)nc(-n3c4ccccc4c4cc5c(cc43)c3ccccc3n5-c3cc4c(cc3-c3ccccc3)c3ccccc3n4-c3ccccc3)n2)cc1]

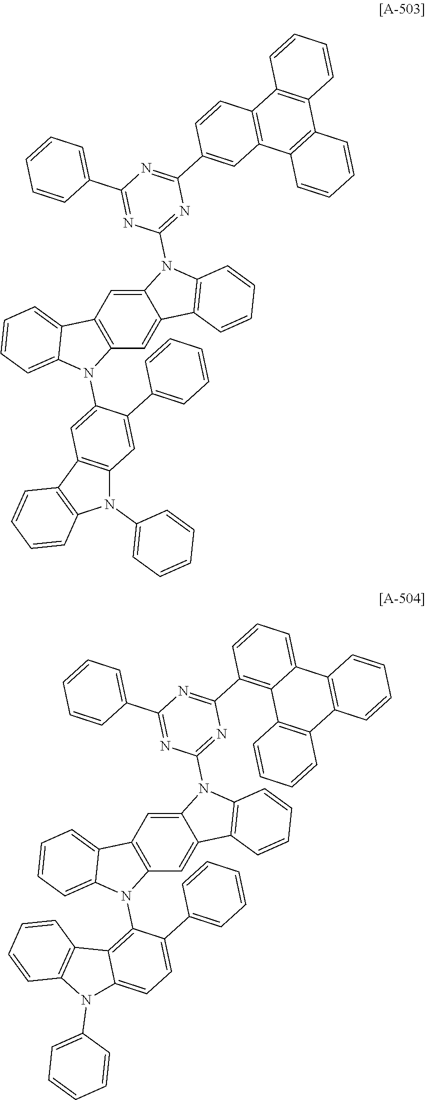 [SMILES: c1ccc(-c2nc(-c3ccc4c5ccccc5c5ccccc5c4c3)nc(-n3c4ccccc4c4cc5c(cc43)c3ccccc3n5-c3cc4c5ccccc5n(-c5ccccc5)c4cc3-c3ccccc3)n2)cc1 ; c1ccc(-c2nc(-c3cccc4c5ccccc5c5ccccc5c34)nc(-n3c4ccccc4c4cc5c(cc43)c3ccccc3n5-c3c(-c4ccccc4)ccc4c3c3ccccc3n4-c3ccccc3)n2)cc1]

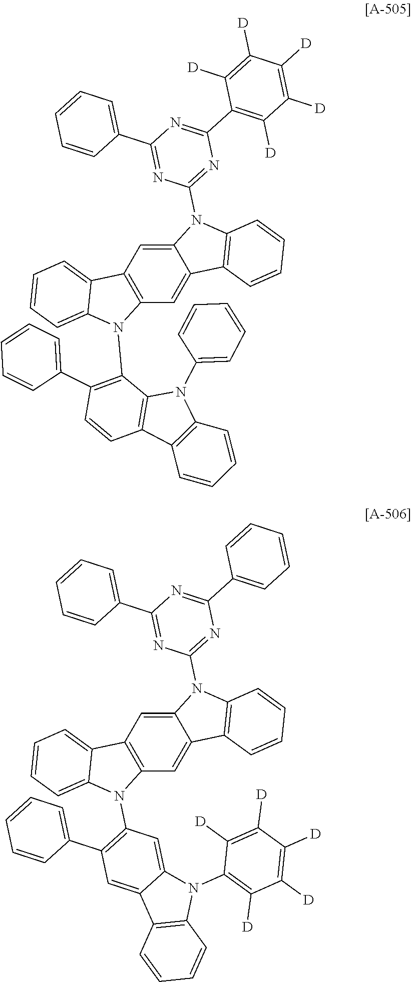 [SMILES: [2H]c1c([2H])c([2H])c(-c2nc(-c3ccccc3)nc(-n3c4ccccc4c4cc5c(cc43)c3ccccc3n5-c3c(-c4ccccc4)ccc4c5ccccc5n(-c5ccccc5)c34)n2)c([2H])c1[2H] ; [2H]c1c([2H])c([2H])c(-n2c3ccccc3c3cc(-c4ccccc4)c(-n4c5ccccc5c5cc6c(cc54)c4ccccc4n6-c4nc(-c5ccccc5)nc(-c5ccccc5)n4)cc32)c([2H])c1[2H]]

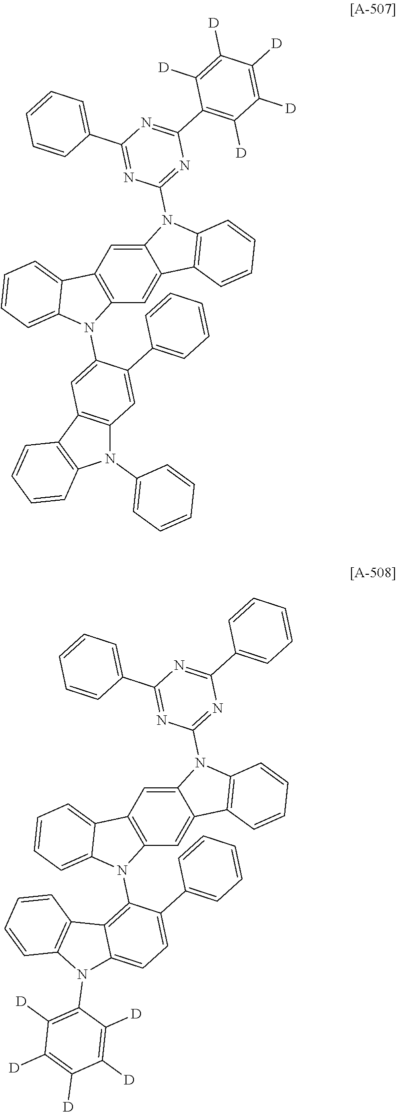 [SMILES: [2H]c1c([2H])c([2H])c(-c2nc(-c3ccccc3)nc(-n3c4ccccc4c4cc5c(cc43)c3ccccc3n5-c3cc4c5ccccc5n(-c5ccccc5)c4cc3-c3ccccc3)n2)c([2H])c1[2H] ; [2H]c1c([2H])c([2H])c(-n2c3ccccc3c3c(-n4c5ccccc5c5cc6c(cc54)c4ccccc4n6-c4nc(-c5ccccc5)nc(-c5ccccc5)n4)c(-c4ccccc4)ccc32)c([2H])c1[2H]]

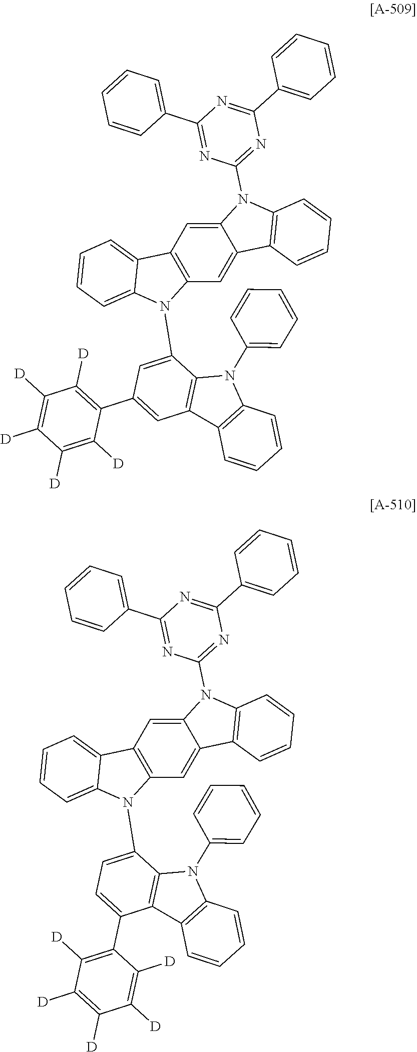 [SMILES: [2H]c1c([2H])c([2H])c(-c2cc(-n3c4ccccc4c4cc5c(cc43)c3ccccc3n5-c3nc(-c4ccccc4)nc(-c4ccccc4)n3)c3c(c2)c2ccccc2n3-c2ccccc2)c([2H])c1[2H] ; [2H]c1c([2H])c([2H])c(-c2ccc(-n3c4ccccc4c4cc5c(cc43)c3ccccc3n5-c3nc(-c4ccccc4)nc(-c4ccccc4)n3)c3c2c2ccccc2n3-c2ccccc2)c([2H])c1[2H]]

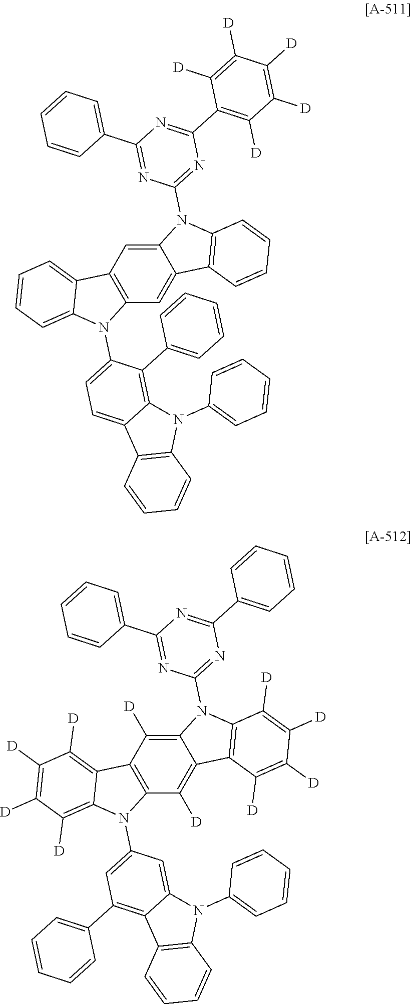 [SMILES: [2H]c1c([2H])c([2H])c(-c2nc(-c3ccccc3)nc(-n3c4ccccc4c4cc5c(cc43)c3ccccc3n5-c3ccc4c5ccccc5n(-c5ccccc5)c4c3-c3ccccc3)n2)c([2H])c1[2H] ; [2H]c1c([2H])c([2H])c2c(c1[2H])c1c([2H])c3c(c([2H])c1n2-c1cc(-c2ccccc2)c2c4ccccc4n(-c4ccccc4)c2c1)c1c([2H])c([2H])c([2H])c([2H])c1n3-c1nc(-c2ccccc2)nc(-c2ccccc2)n1]

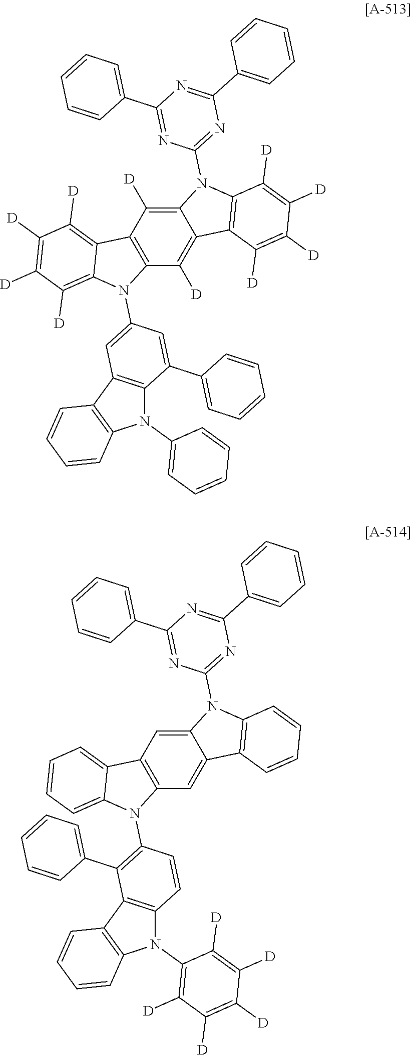 [SMILES: [2H]c1c([2H])c([2H])c2c(c1[2H])c1c([2H])c3c(c([2H])c1n2-c1cc(-c2ccccc2)c2c(c1)c1ccccc1n2-c1ccccc1)c1c([2H])c([2H])c([2H])c([2H])c1n3-c1nc(-c2ccccc2)nc(-c2ccccc2)n1 ; [2H]c1c([2H])c([2H])c(-n2c3ccccc3c3c(-c4ccccc4)c(-n4c5ccccc5c5cc6c(cc54)c4ccccc4n6-c4nc(-c5ccccc5)nc(-c5ccccc5)n4)ccc32)c([2H])c1[2H]]

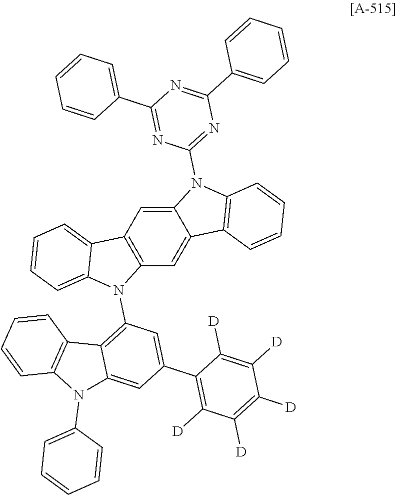 [SMILES: [2H]c1c([2H])c([2H])c(-c2cc(-n3c4ccccc4c4cc5c(cc43)c3ccccc3n5-c3nc(-c4ccccc4)nc(-c4ccccc4)n3)c3c4ccccc4n(-c4ccccc4)c3c2)c([2H])c1[2H]]

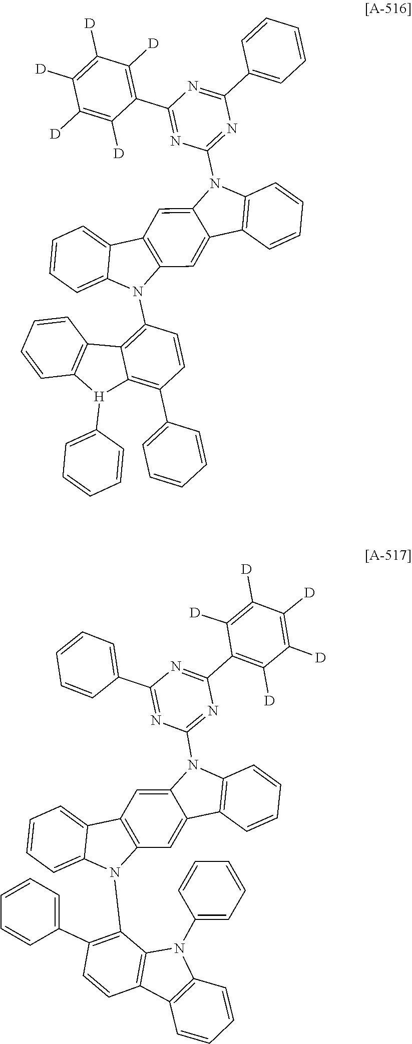

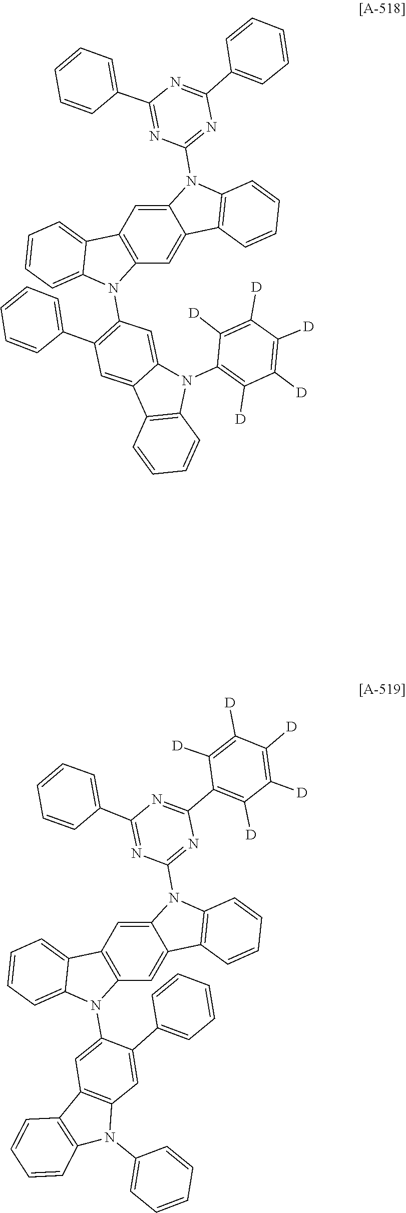 [SMILES: [2H]c1c([2H])c([2H])c(-n2c3ccccc3c3cc(-c4ccccc4)c(-n4c5ccccc5c5cc6c(cc54)c4ccccc4n6-c4nc(-c5ccccc5)nc(-c5ccccc5)n4)cc32)c([2H])c1[2H] ; [2H]c1c([2H])c([2H])c(-c2nc(-c3ccccc3)nc(-n3c4ccccc4c4cc5c(cc43)c3ccccc3n5-c3cc4c5ccccc5n(-c5ccccc5)c4cc3-c3ccccc3)n2)c([2H])c1[2H]]

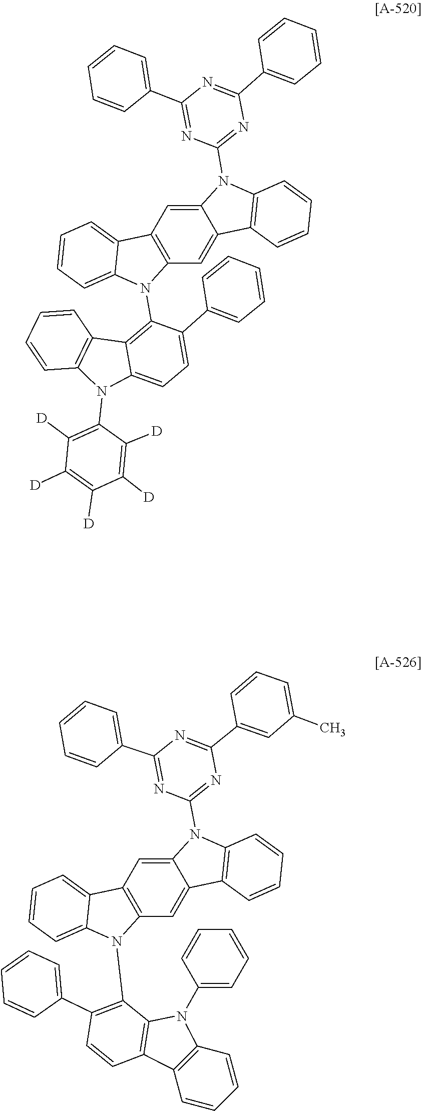 [SMILES: [2H]c1c([2H])c([2H])c(-n2c3ccccc3c3c(-n4c5ccccc5c5cc6c(cc54)c4ccccc4n6-c4nc(-c5ccccc5)nc(-c5ccccc5)n4)c(-c4ccccc4)ccc32)c([2H])c1[2H] ; Cc1cccc(-c2nc(-c3ccccc3)nc(-n3c4ccccc4c4cc5c(cc43)c3ccccc3n5-c3c(-c4ccccc4)ccc4c5ccccc5n(-c5ccccc5)c34)n2)c1]

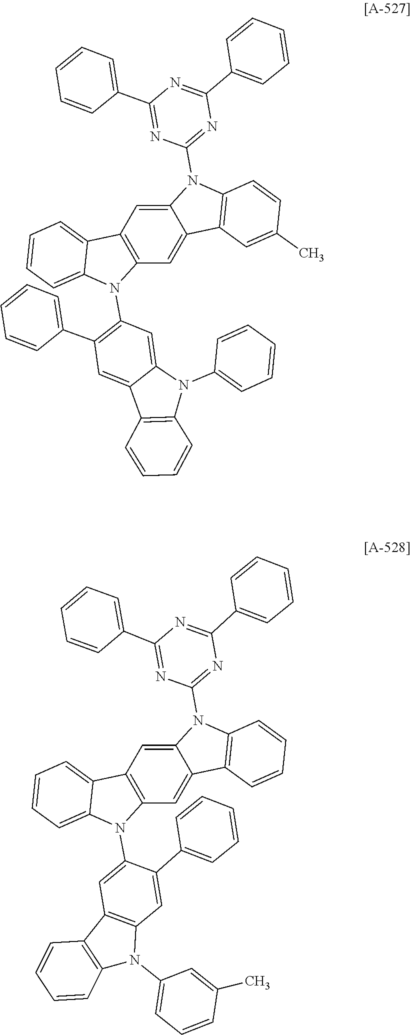 [SMILES: Cc1ccc2c(c1)c1cc3c(cc1n2-c1nc(-c2ccccc2)nc(-c2ccccc2)n1)c1ccccc1n3-c1cc2c(cc1-c1ccccc1)c1ccccc1n2-c1ccccc1 ; Cc1cccc(-n2c3ccccc3c3cc(-n4c5ccccc5c5cc6c(cc54)c4ccccc4n6-c4nc(-c5ccccc5)nc(-c5ccccc5)n4)c(-c4ccccc4)cc32)c1]

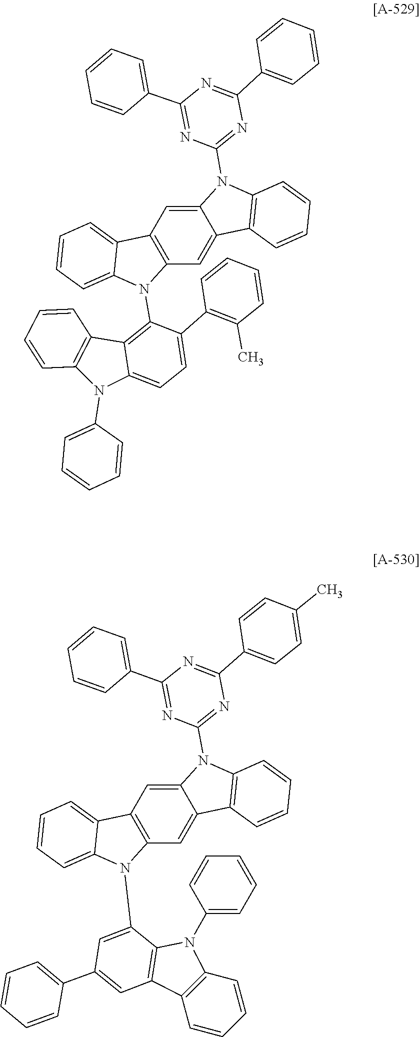 [SMILES: Cc1ccccc1-c1ccc2c(c1-n1c3ccccc3c3cc4c(cc31)c1ccccc1n4-c1nc(-c3ccccc3)nc(-c3ccccc3)n1)c1ccccc1n2-c1ccccc1 ; Cc1ccc(-c2nc(-c3ccccc3)nc(-n3c4ccccc4c4cc5c(cc43)c3ccccc3n5-c3cc(-c4ccccc4)cc4c5ccccc5n(-c5ccccc5)c34)n2)cc1]

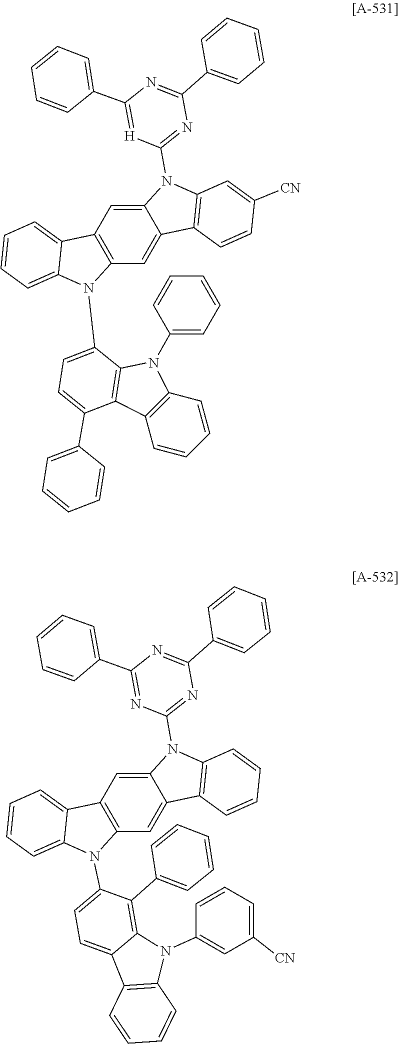

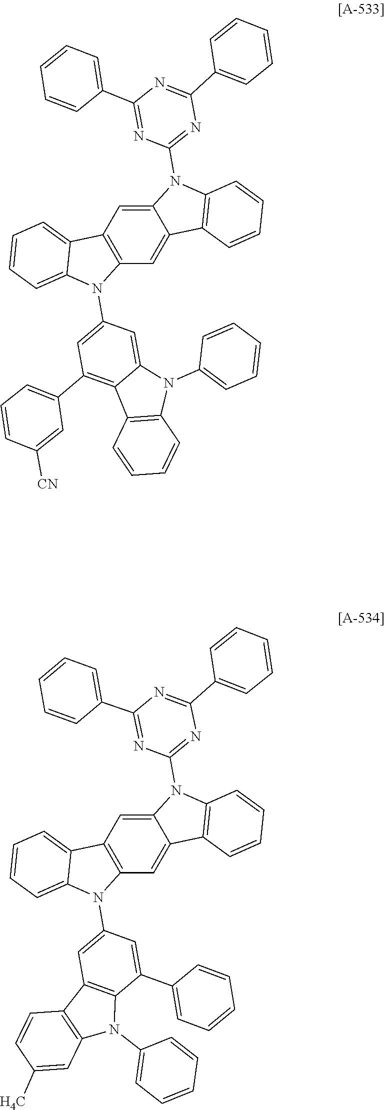

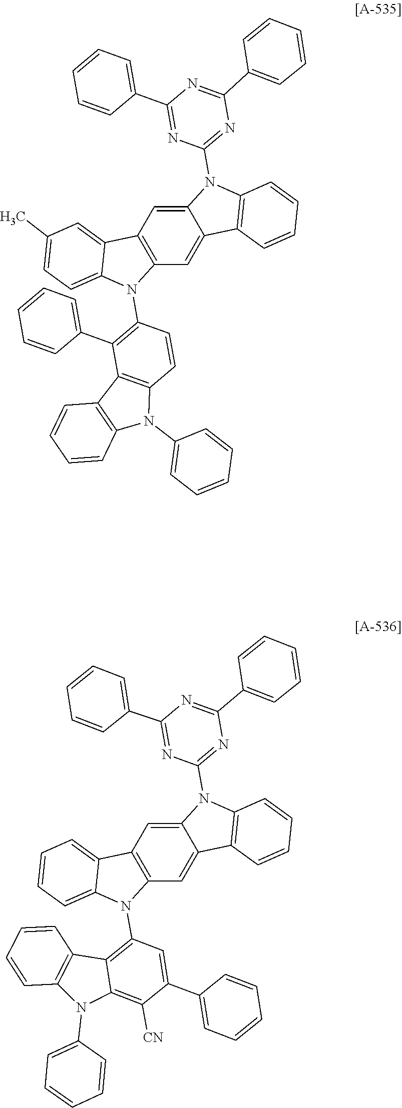 [SMILES: Cc1ccc2c(c1)c1cc3c(cc1n2-c1ccc2c(c1-c1ccccc1)c1ccccc1n2-c1ccccc1)c1ccccc1n3-c1nc(-c2ccccc2)nc(-c2ccccc2)n1 ; N#Cc1c(-c2ccccc2)cc(-n2c3ccccc3c3cc4c(cc32)c2ccccc2n4-c2nc(-c3ccccc3)nc(-c3ccccc3)n2)c2c3ccccc3n(-c3ccccc3)c12]

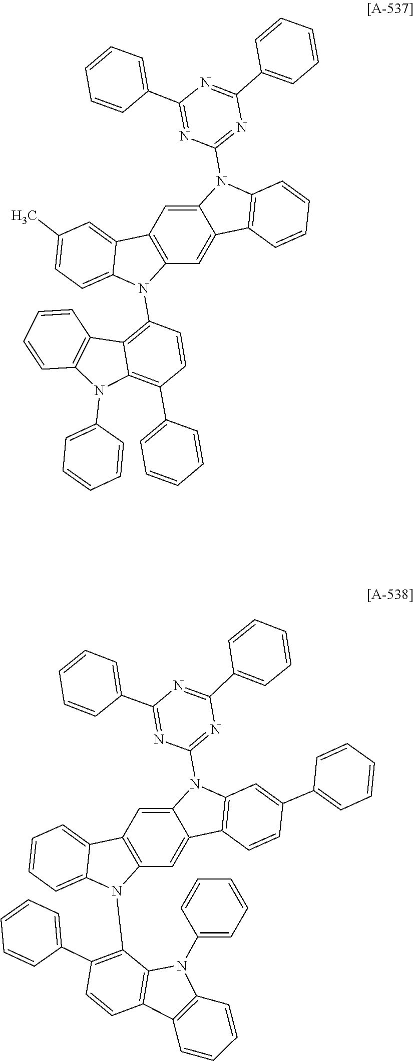 [SMILES: Cc1ccc2c(c1)c1cc3c(cc1n2-c1ccc(-c2ccccc2)c2c1c1ccccc1n2-c1ccccc1)c1ccccc1n3-c1nc(-c2ccccc2)nc(-c2ccccc2)n1 ; c1ccc(-c2ccc3c4cc5c(cc4n(-c4nc(-c6ccccc6)nc(-c6ccccc6)n4)c3c2)c2ccccc2n5-c2c(-c3ccccc3)ccc3c4ccccc4n(-c4ccccc4)c23)cc1]

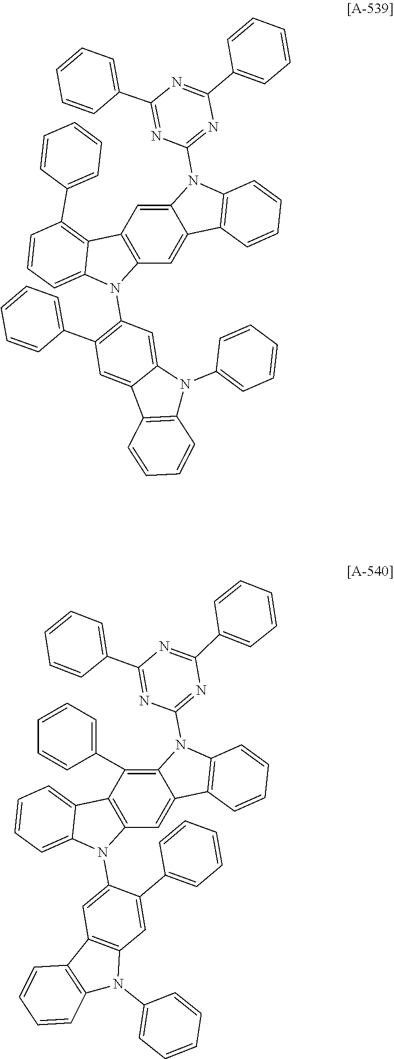 [SMILES: c1ccc(-c2nc(-c3ccccc3)nc(-n3c4ccccc4c4cc5c(cc43)c3c(-c4ccccc4)cccc3n5-c3cc4c(cc3-c3ccccc3)c3ccccc3n4-c3ccccc3)n2)cc1 ; c1ccc(-c2nc(-c3ccccc3)nc(-n3c4ccccc4c4cc5c(c(-c6ccccc6)c43)c3ccccc3n5-c3cc4c5ccccc5n(-c5ccccc5)c4cc3-c3ccccc3)n2)cc1]

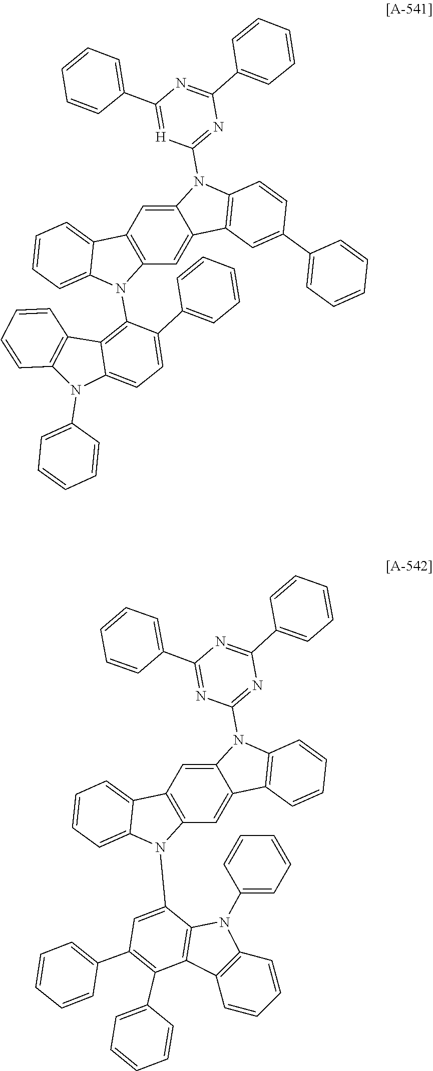

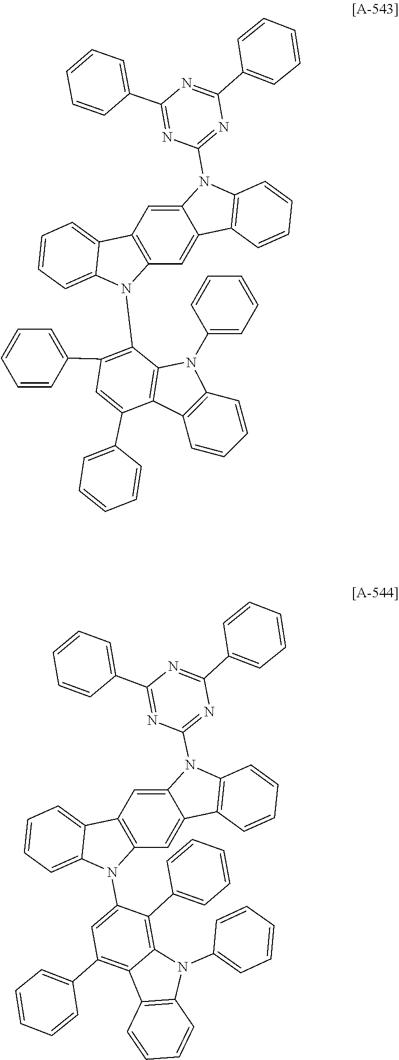 [SMILES: c1ccc(-c2nc(-c3ccccc3)nc(-n3c4ccccc4c4cc5c(cc43)c3ccccc3n5-c3c(-c4ccccc4)cc(-c4ccccc4)c4c5ccccc5n(-c5ccccc5)c34)n2)cc1 ; c1ccc(-c2nc(-c3ccccc3)nc(-n3c4ccccc4c4cc5c(cc43)c3ccccc3n5-c3cc(-c4ccccc4)c4c5ccccc5n(-c5ccccc5)c4c3-c3ccccc3)n2)cc1]

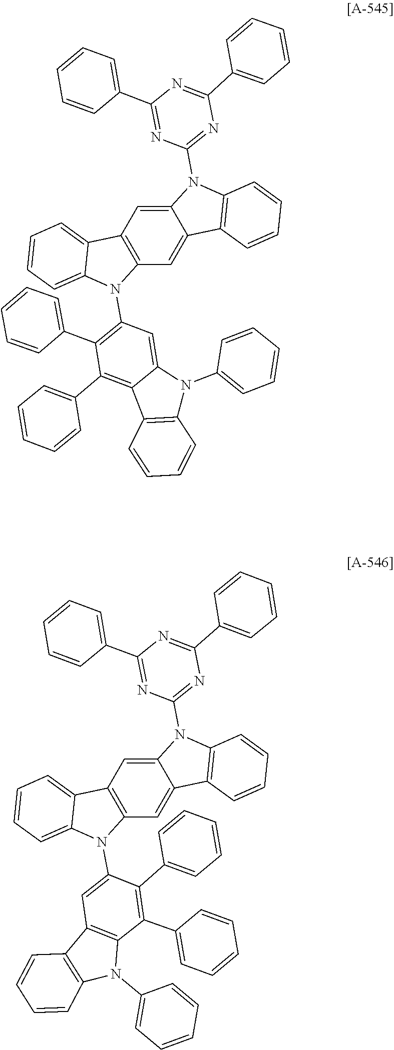 [SMILES: c1ccc(-c2nc(-c3ccccc3)nc(-n3c4ccccc4c4cc5c(cc43)c3ccccc3n5-c3cc4c(c(-c5ccccc5)c3-c3ccccc3)c3ccccc3n4-c3ccccc3)n2)cc1 ; c1ccc(-c2nc(-c3ccccc3)nc(-n3c4ccccc4c4cc5c(cc43)c3ccccc3n5-c3cc4c5ccccc5n(-c5ccccc5)c4c(-c4ccccc4)c3-c3ccccc3)n2)cc1]

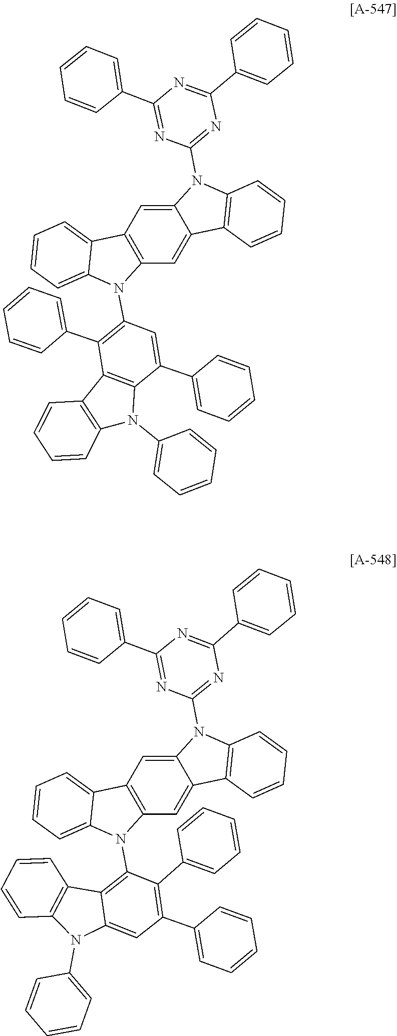 [SMILES: c1ccc(-c2nc(-c3ccccc3)nc(-n3c4ccccc4c4cc5c(cc43)c3ccccc3n5-c3cc(-c4ccccc4)c4c(c3-c3ccccc3)c3ccccc3n4-c3ccccc3)n2)cc1 ; c1ccc(-c2nc(-c3ccccc3)nc(-n3c4ccccc4c4cc5c(cc43)c3ccccc3n5-c3c(-c4ccccc4)c(-c4ccccc4)cc4c3c3ccccc3n4-c3ccccc3)n2)cc1]

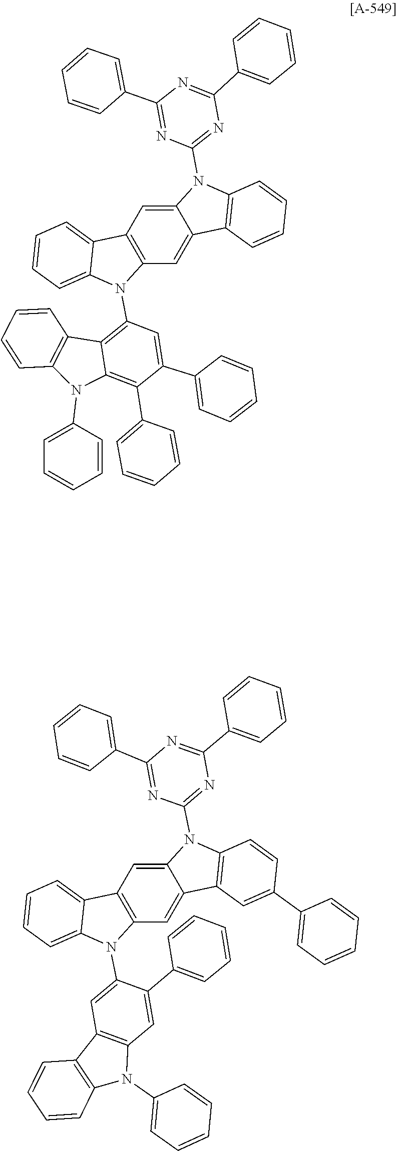 [SMILES: c1ccc(-c2nc(-c3ccccc3)nc(-n3c4ccccc4c4cc5c(cc43)c3ccccc3n5-c3cc(-c4ccccc4)c(-c4ccccc4)c4c3c3ccccc3n4-c3ccccc3)n2)cc1 ; c1ccc(-c2ccc3c(c2)c2cc4c(cc2n3-c2nc(-c3ccccc3)nc(-c3ccccc3)n2)c2ccccc2n4-c2cc3c4ccccc4n(-c4ccccc4)c3cc2-c2ccccc2)cc1]

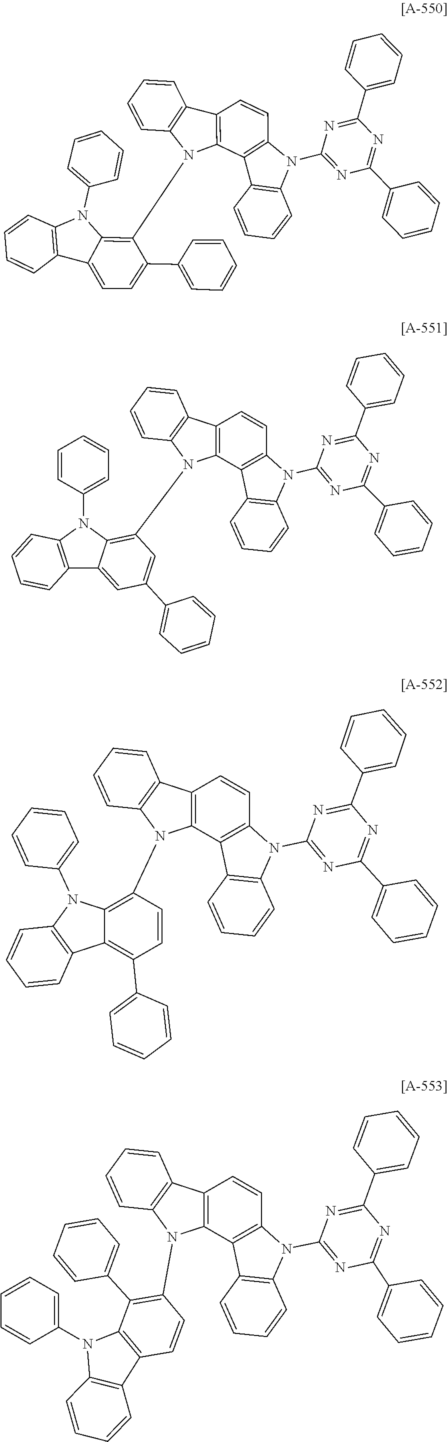 [SMILES: c1ccc(-c2nc(-c3ccccc3)nc(-n3c4ccccc4c4c3ccc3c5ccccc5n(-c5c(-c6ccccc6)ccc6c7ccccc7n(-c7ccccc7)c56)c34)n2)cc1 ; c1ccc(-c2cc(-n3c4ccccc4c4ccc5c(c6ccccc6n5-c5nc(-c6ccccc6)nc(-c6ccccc6)n5)c43)c3c(c2)c2ccccc2n3-c2ccccc2)cc1 ; c1ccc(-c2nc(-c3ccccc3)nc(-n3c4ccccc4c4c3ccc3c5ccccc5n(-c5ccc(-c6ccccc6)c6c7ccccc7n(-c7ccccc7)c56)c34)n2)cc1 ; c1ccc(-c2nc(-c3ccccc3)nc(-n3c4ccccc4c4c3ccc3c5ccccc5n(-c5ccc6c7ccccc7n(-c7ccccc7)c6c5-c5ccccc5)c34)n2)cc1]

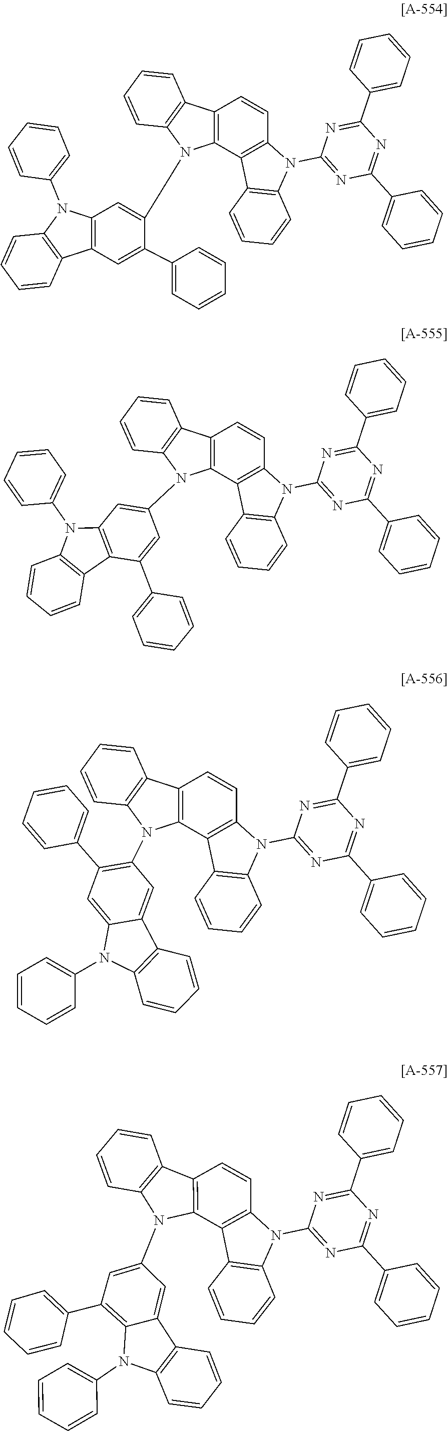 [SMILES: c1ccc(-c2nc(-c3ccccc3)nc(-n3c4ccccc4c4c3ccc3c5ccccc5n(-c5cc6c(cc5-c5ccccc5)c5ccccc5n6-c5ccccc5)c34)n2)cc1 ; c1ccc(-c2nc(-c3ccccc3)nc(-n3c4ccccc4c4c3ccc3c5ccccc5n(-c5cc(-c6ccccc6)c6c7ccccc7n(-c7ccccc7)c6c5)c34)n2)cc1 ; c1ccc(-c2nc(-c3ccccc3)nc(-n3c4ccccc4c4c3ccc3c5ccccc5n(-c5cc6c7ccccc7n(-c7ccccc7)c6cc5-c5ccccc5)c34)n2)cc1 ; c1ccc(-c2nc(-c3ccccc3)nc(-n3c4ccccc4c4c3ccc3c5ccccc5n(-c5cc(-c6ccccc6)c6c(c5)c5ccccc5n6-c5ccccc5)c34)n2)cc1]

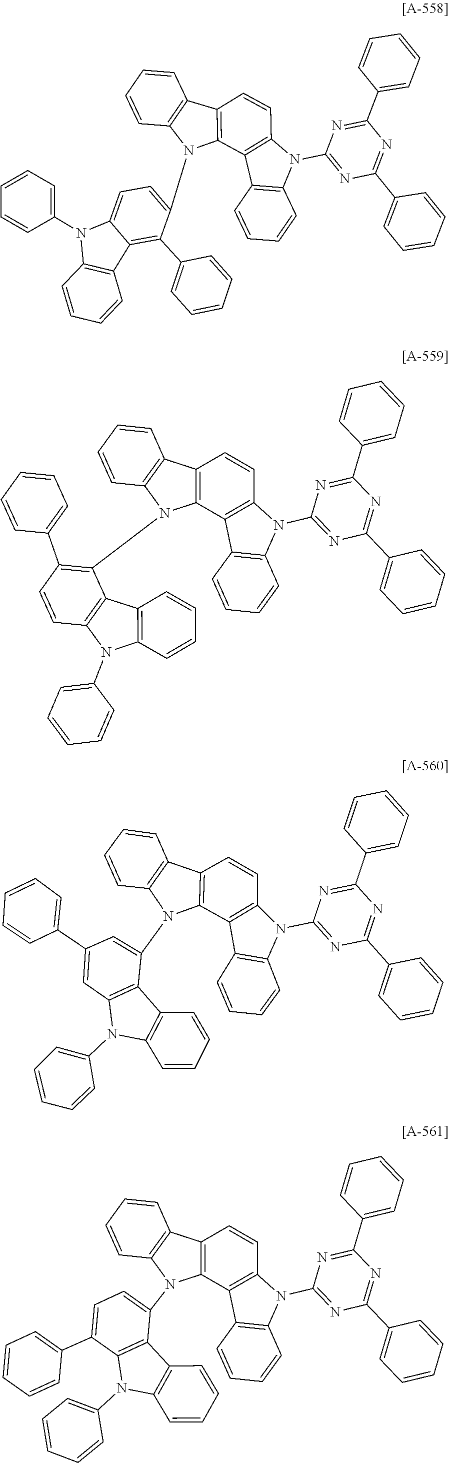 [SMILES: c1ccc(-c2nc(-c3ccccc3)nc(-n3c4ccccc4c4c3ccc3c5ccccc5n(-c5ccc6c(c5-c5ccccc5)c5ccccc5n6-c5ccccc5)c34)n2)cc1 ; c1ccc(-c2nc(-c3ccccc3)nc(-n3c4ccccc4c4c3ccc3c5ccccc5n(-c5c(-c6ccccc6)ccc6c5c5ccccc5n6-c5ccccc5)c34)n2)cc1 ; c1ccc(-c2cc(-n3c4ccccc4c4ccc5c(c6ccccc6n5-c5nc(-c6ccccc6)nc(-c6ccccc6)n5)c43)c3c4ccccc4n(-c4ccccc4)c3c2)cc1 ; c1ccc(-c2nc(-c3ccccc3)nc(-n3c4ccccc4c4c3ccc3c5ccccc5n(-c5ccc(-c6ccccc6)c6c5c5ccccc5n6-c5ccccc5)c34)n2)cc1]

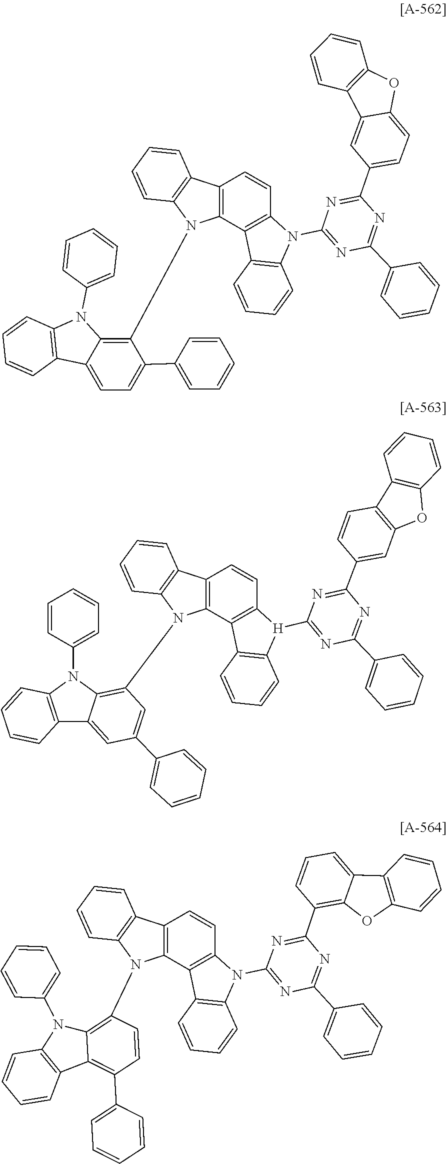

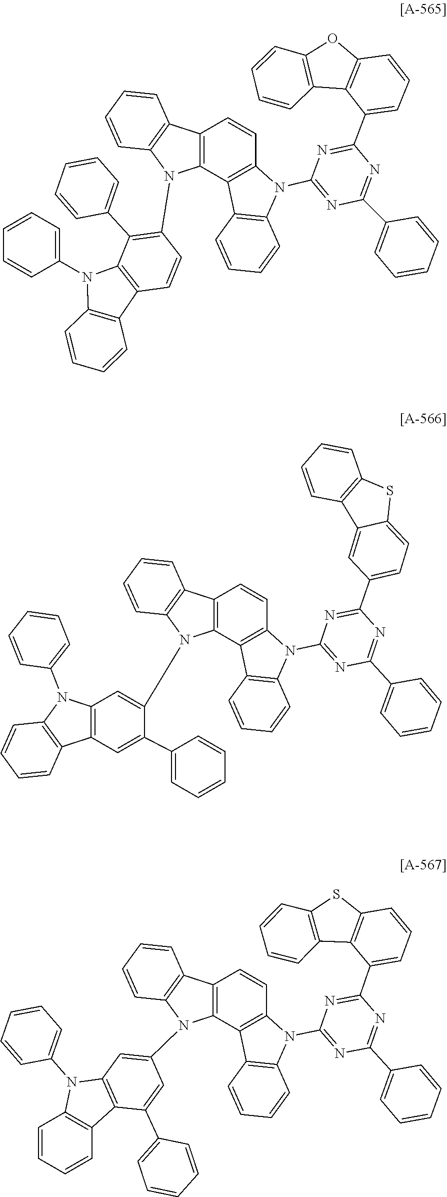 [SMILES: c1ccc(-c2nc(-c3cccc4oc5ccccc5c34)nc(-n3c4ccccc4c4c3ccc3c5ccccc5n(-c5ccc6c7ccccc7n(-c7ccccc7)c6c5-c5ccccc5)c34)n2)cc1 ; c1ccc(-c2nc(-c3ccc4sc5ccccc5c4c3)nc(-n3c4ccccc4c4c3ccc3c5ccccc5n(-c5cc6c(cc5-c5ccccc5)c5ccccc5n6-c5ccccc5)c34)n2)cc1 ; c1ccc(-c2nc(-c3cccc4sc5ccccc5c34)nc(-n3c4ccccc4c4c3ccc3c5ccccc5n(-c5cc(-c6ccccc6)c6c7ccccc7n(-c7ccccc7)c6c5)c34)n2)cc1]

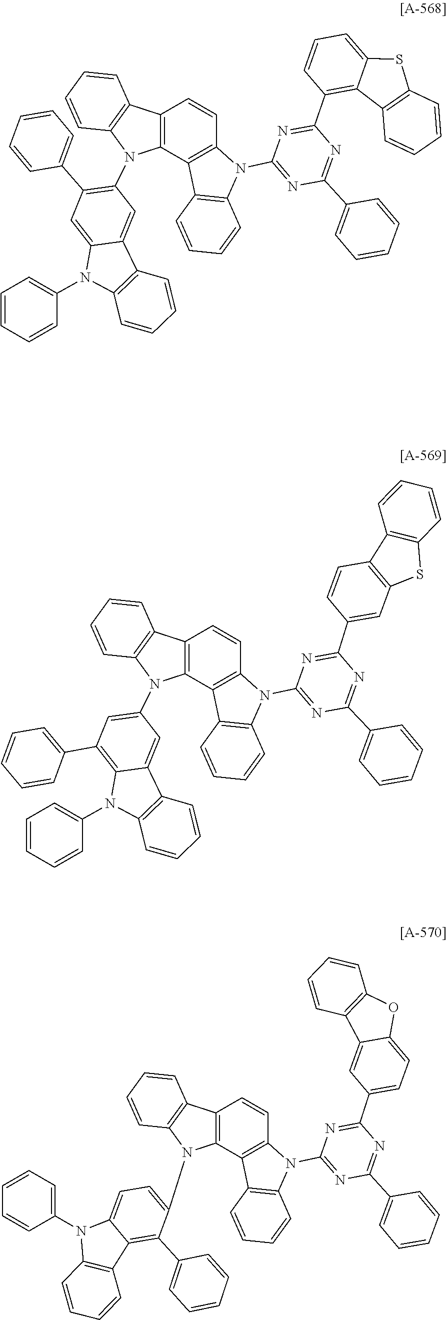 [SMILES: c1ccc(-c2nc(-c3cccc4sc5ccccc5c34)nc(-n3c4ccccc4c4c3ccc3c5ccccc5n(-c5cc6c7ccccc7n(-c7ccccc7)c6cc5-c5ccccc5)c34)n2)cc1 ; c1ccc(-c2nc(-c3ccc4c(c3)sc3ccccc34)nc(-n3c4ccccc4c4c3ccc3c5ccccc5n(-c5cc(-c6ccccc6)c6c(c5)c5ccccc5n6-c5ccccc5)c34)n2)cc1 ; c1ccc(-c2nc(-c3ccc4oc5ccccc5c4c3)nc(-n3c4ccccc4c4c3ccc3c5ccccc5n(-c5ccc6c(c5-c5ccccc5)c5ccccc5n6-c5ccccc5)c34)n2)cc1]

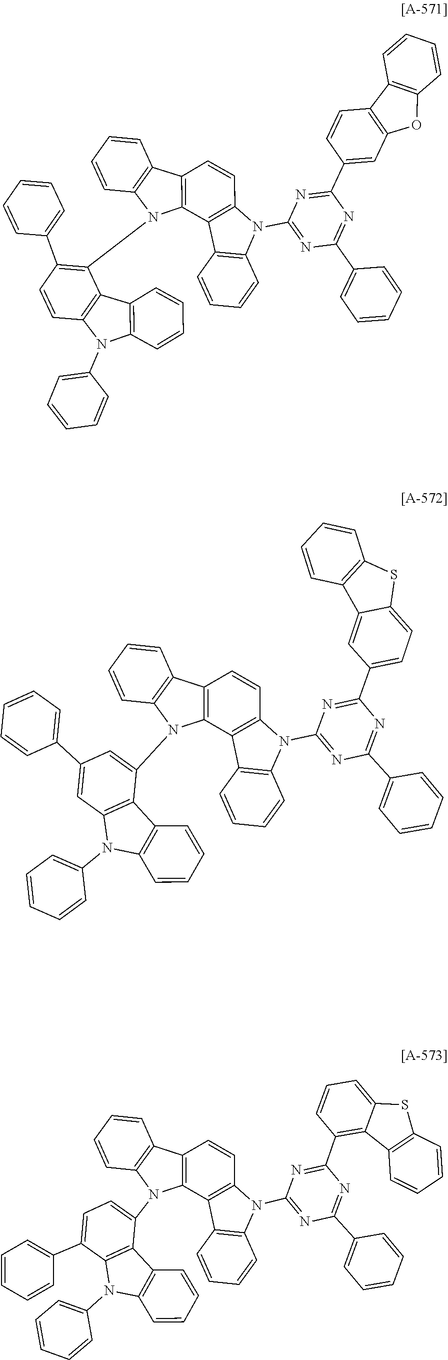 [SMILES: c1ccc(-c2nc(-c3ccc4c(c3)oc3ccccc34)nc(-n3c4ccccc4c4c3ccc3c5ccccc5n(-c5c(-c6ccccc6)ccc6c5c5ccccc5n6-c5ccccc5)c34)n2)cc1 ; c1ccc(-c2cc(-n3c4ccccc4c4ccc5c(c6ccccc6n5-c5nc(-c6ccccc6)nc(-c6ccc7sc8ccccc8c7c6)n5)c43)c3c4ccccc4n(-c4ccccc4)c3c2)cc1 ; c1ccc(-c2nc(-c3cccc4sc5ccccc5c34)nc(-n3c4ccccc4c4c3ccc3c5ccccc5n(-c5ccc(-c6ccccc6)c6c5c5ccccc5n6-c5ccccc5)c34)n2)cc1]

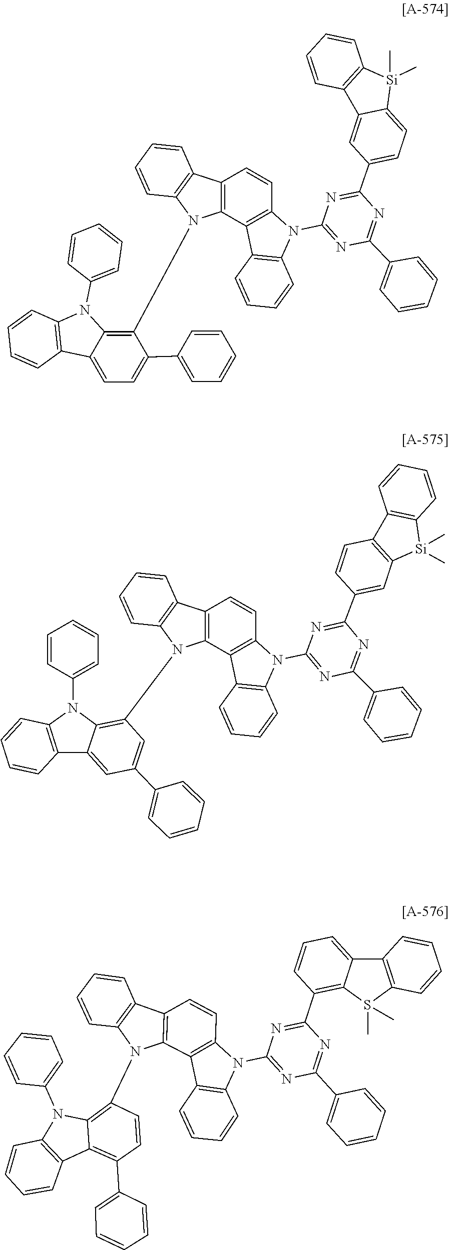 [SMILES: C[Si]1(C)c2ccccc2-c2cc(-c3nc(-c4ccccc4)nc(-n4c5ccccc5c5c4ccc4c6ccccc6n(-c6c(-c7ccccc7)ccc7c8ccccc8n(-c8ccccc8)c67)c45)n3)ccc21 ; C[Si]1(C)c2ccccc2-c2ccc(-c3nc(-c4ccccc4)nc(-n4c5ccccc5c5c4ccc4c6ccccc6n(-c6cc(-c7ccccc7)cc7c8ccccc8n(-c8ccccc8)c67)c45)n3)cc21 ; CS1(C)c2ccccc2-c2cccc(-c3nc(-c4ccccc4)nc(-n4c5ccccc5c5c4ccc4c6ccccc6n(-c6ccc(-c7ccccc7)c7c8ccccc8n(-c8ccccc8)c67)c45)n3)c21]

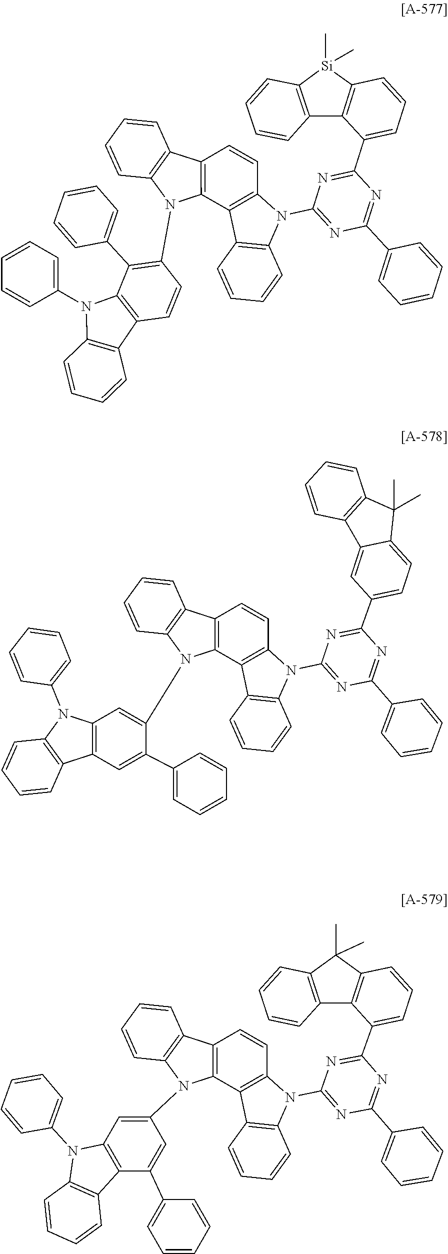

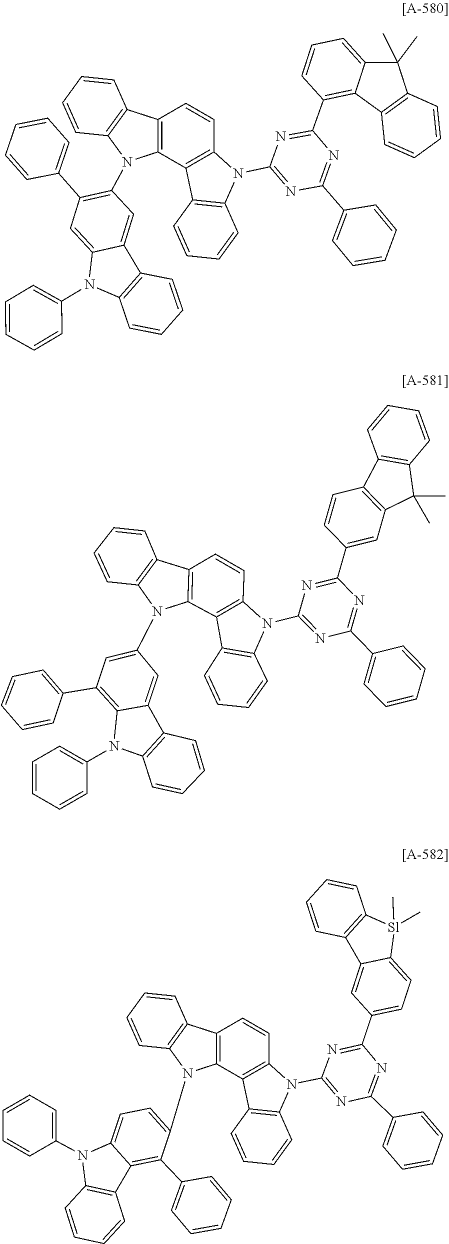

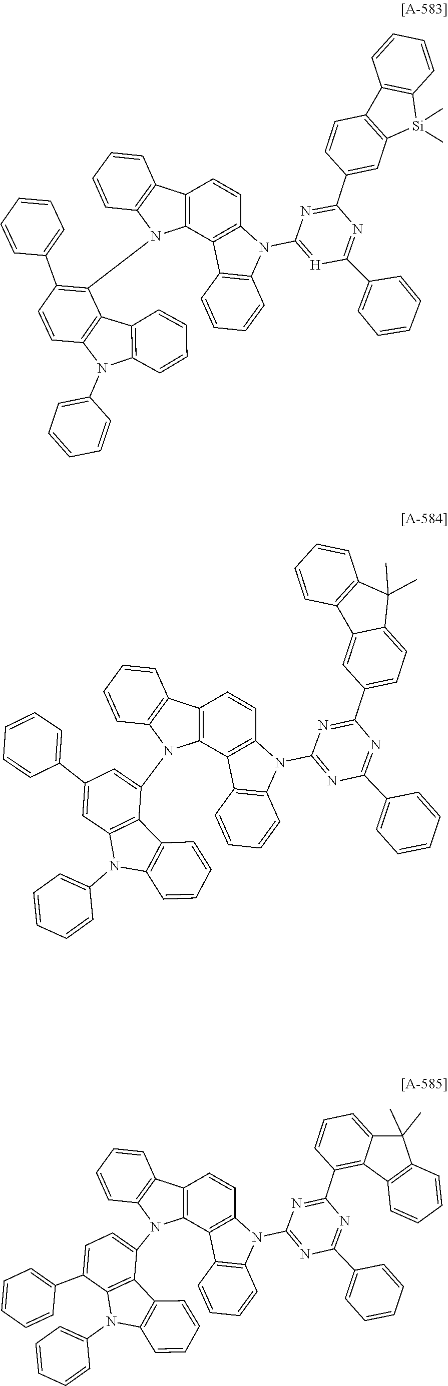

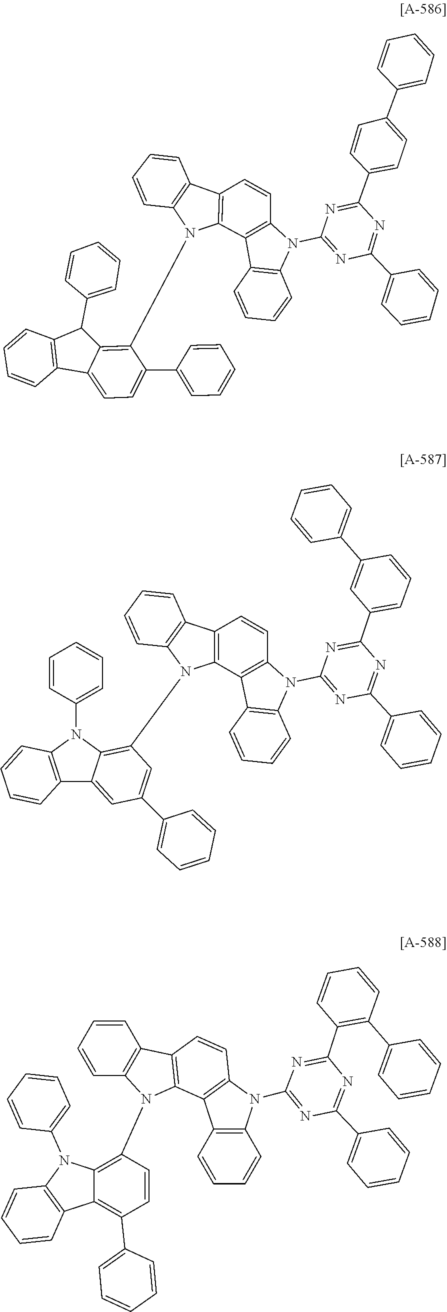 [SMILES: c1ccc(-c2ccc(-c3nc(-c4ccccc4)nc(-n4c5ccccc5c5c4ccc4c6ccccc6n(-c6c(-c7ccccc7)ccc7c6C(c6ccccc6)c6ccccc6-7)c45)n3)cc2)cc1 ; c1ccc(-c2cccc(-c3nc(-c4ccccc4)nc(-n4c5ccccc5c5c4ccc4c6ccccc6n(-c6cc(-c7ccccc7)cc7c8ccccc8n(-c8ccccc8)c67)c45)n3)c2)cc1 ; c1ccc(-c2nc(-c3ccccc3-c3ccccc3)nc(-n3c4ccccc4c4c3ccc3c5ccccc5n(-c5ccc(-c6ccccc6)c6c7ccccc7n(-c7ccccc7)c56)c34)n2)cc1]

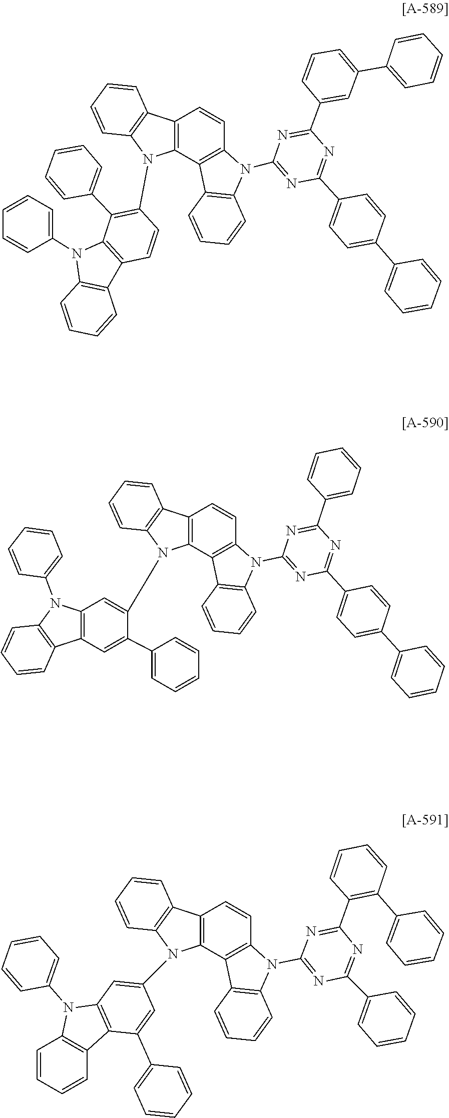 [SMILES: c1ccc(-c2ccc(-c3nc(-c4cccc(-c5ccccc5)c4)nc(-n4c5ccccc5c5c4ccc4c6ccccc6n(-c6ccc7c8ccccc8n(-c8ccccc8)c7c6-c6ccccc6)c45)n3)cc2)cc1 ; c1ccc(-c2ccc(-c3nc(-c4ccccc4)nc(-n4c5ccccc5c5c4ccc4c6ccccc6n(-c6cc7c(cc6-c6ccccc6)c6ccccc6n7-c6ccccc6)c45)n3)cc2)cc1 ; c1ccc(-c2nc(-c3ccccc3-c3ccccc3)nc(-n3c4ccccc4c4c3ccc3c5ccccc5n(-c5cc(-c6ccccc6)c6c7ccccc7n(-c7ccccc7)c6c5)c34)n2)cc1]

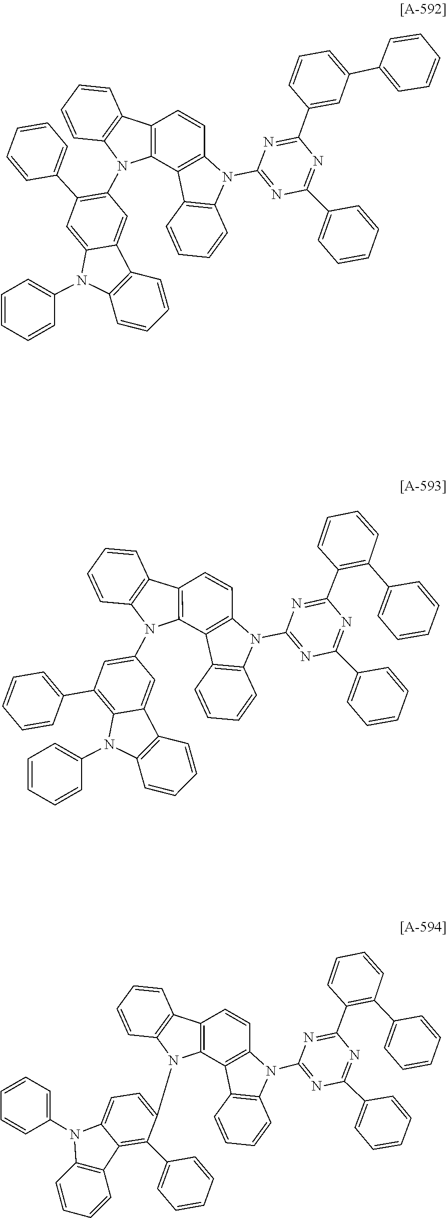 [SMILES: c1ccc(-c2cccc(-c3nc(-c4ccccc4)nc(-n4c5ccccc5c5c4ccc4c6ccccc6n(-c6cc7c8ccccc8n(-c8ccccc8)c7cc6-c6ccccc6)c45)n3)c2)cc1 ; c1ccc(-c2nc(-c3ccccc3-c3ccccc3)nc(-n3c4ccccc4c4c3ccc3c5ccccc5n(-c5cc(-c6ccccc6)c6c(c5)c5ccccc5n6-c5ccccc5)c34)n2)cc1 ; c1ccc(-c2nc(-c3ccccc3-c3ccccc3)nc(-n3c4ccccc4c4c3ccc3c5ccccc5n(-c5ccc6c(c5-c5ccccc5)c5ccccc5n6-c5ccccc5)c34)n2)cc1]

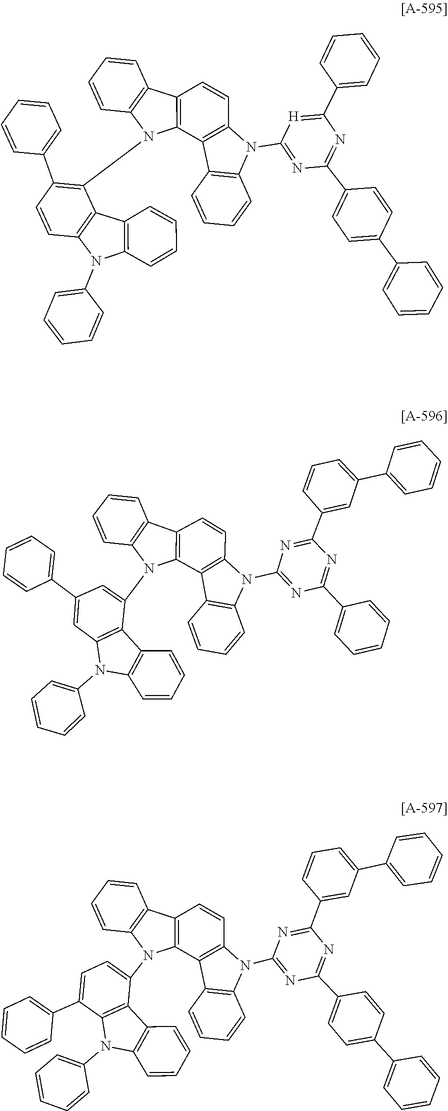

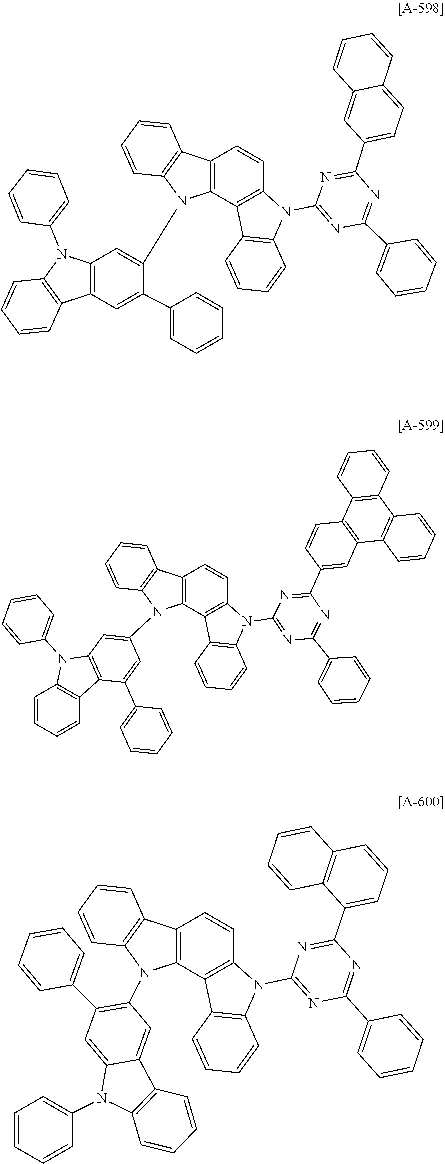 [SMILES: c1ccc(-c2nc(-c3ccc4ccccc4c3)nc(-n3c4ccccc4c4c3ccc3c5ccccc5n(-c5cc6c(cc5-c5ccccc5)c5ccccc5n6-c5ccccc5)c34)n2)cc1 ; c1ccc(-c2nc(-c3ccc4c5ccccc5c5ccccc5c4c3)nc(-n3c4ccccc4c4c3ccc3c5ccccc5n(-c5cc(-c6ccccc6)c6c7ccccc7n(-c7ccccc7)c6c5)c34)n2)cc1 ; c1ccc(-c2nc(-c3cccc4ccccc34)nc(-n3c4ccccc4c4c3ccc3c5ccccc5n(-c5cc6c7ccccc7n(-c7ccccc7)c6cc5-c5ccccc5)c34)n2)cc1]

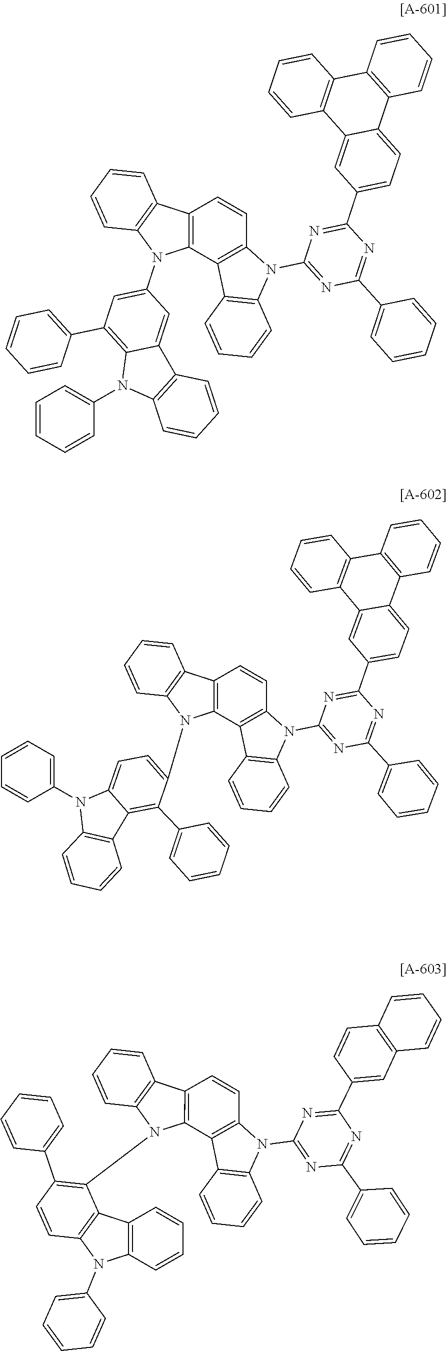 [SMILES: c1ccc(-c2nc(-c3ccc4c5ccccc5c5ccccc5c4c3)nc(-n3c4ccccc4c4c3ccc3c5ccccc5n(-c5cc(-c6ccccc6)c6c(c5)c5ccccc5n6-c5ccccc5)c34)n2)cc1 ; c1ccc(-c2nc(-c3ccc4c5ccccc5c5ccccc5c4c3)nc(-n3c4ccccc4c4c3ccc3c5ccccc5n(-c5ccc6c(c5-c5ccccc5)c5ccccc5n6-c5ccccc5)c34)n2)cc1 ; c1ccc(-c2nc(-c3ccc4ccccc4c3)nc(-n3c4ccccc4c4c3ccc3c5ccccc5n(-c5c(-c6ccccc6)ccc6c5c5ccccc5n6-c5ccccc5)c34)n2)cc1]

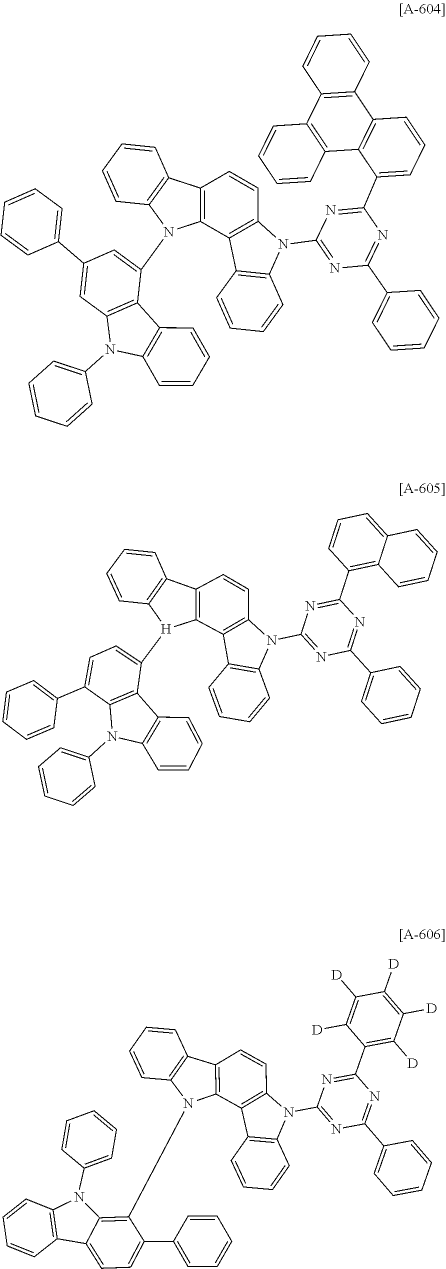

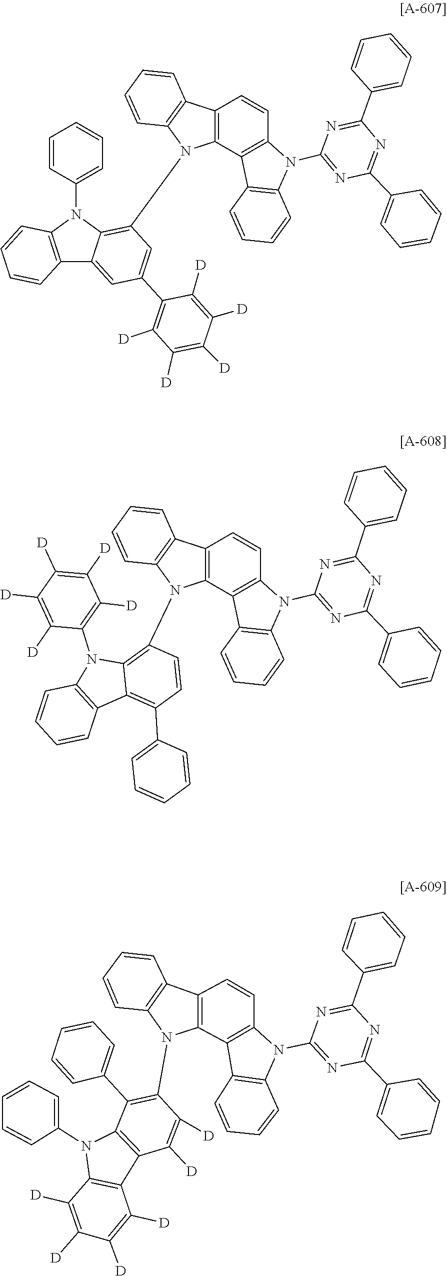 [SMILES: [2H]c1c([2H])c([2H])c(-c2cc(-n3c4ccccc4c4ccc5c(c6ccccc6n5-c5nc(-c6ccccc6)nc(-c6ccccc6)n5)c43)c3c(c2)c2ccccc2n3-c2ccccc2)c([2H])c1[2H] ; [2H]c1c([2H])c([2H])c(-n2c3ccccc3c3c(-c4ccccc4)ccc(-n4c5ccccc5c5ccc6c(c7ccccc7n6-c6nc(-c7ccccc7)nc(-c7ccccc7)n6)c54)c32)c([2H])c1[2H] ; [2H]c1c([2H])c([2H])c2c(c1[2H])c1c([2H])c([2H])c(-n3c4ccccc4c4ccc5c(c6ccccc6n5-c5nc(-c6ccccc6)nc(-c6ccccc6)n5)c43)c(-c3ccccc3)c1n2-c1ccccc1]

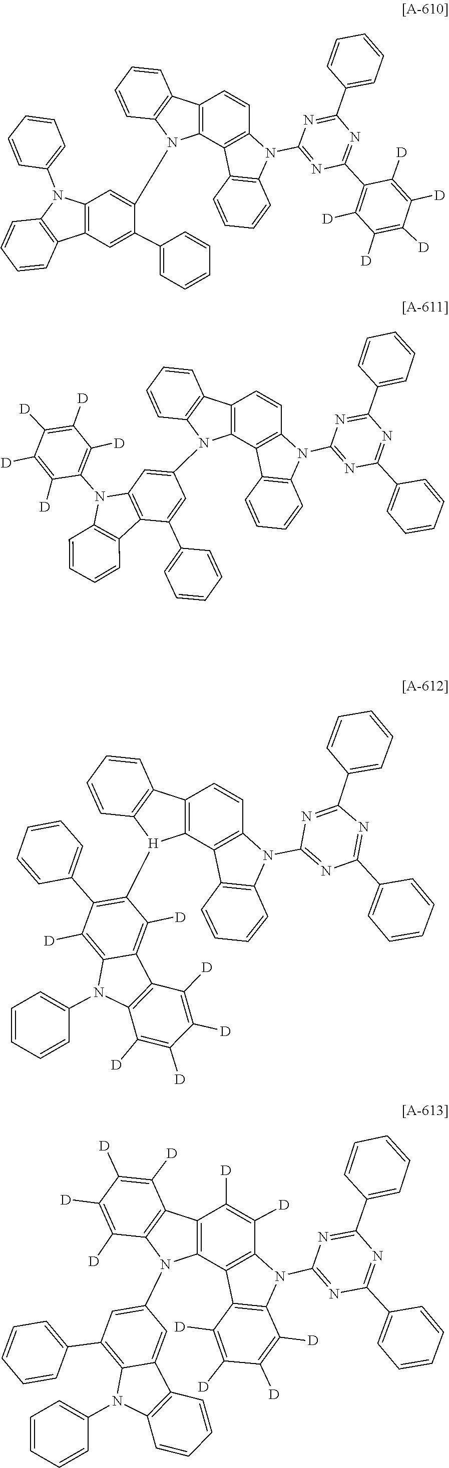

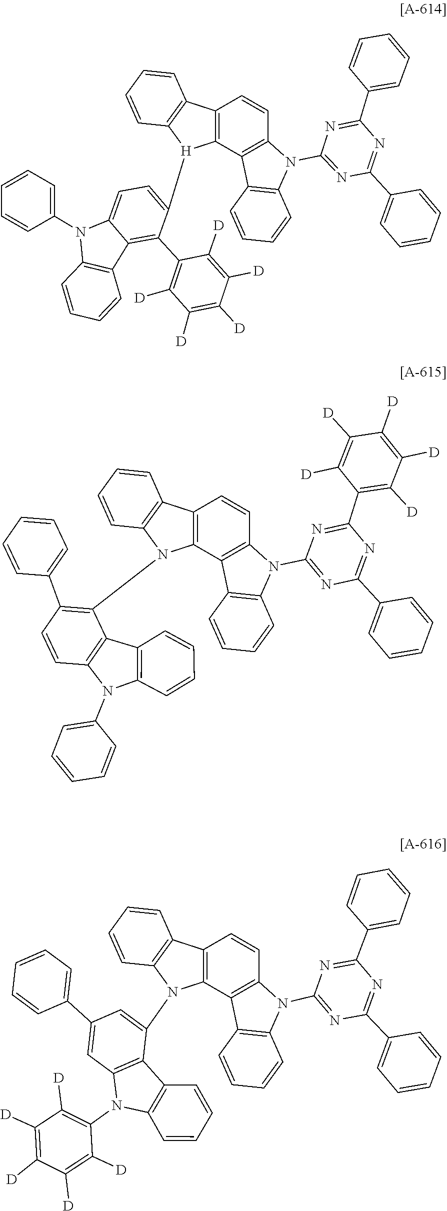

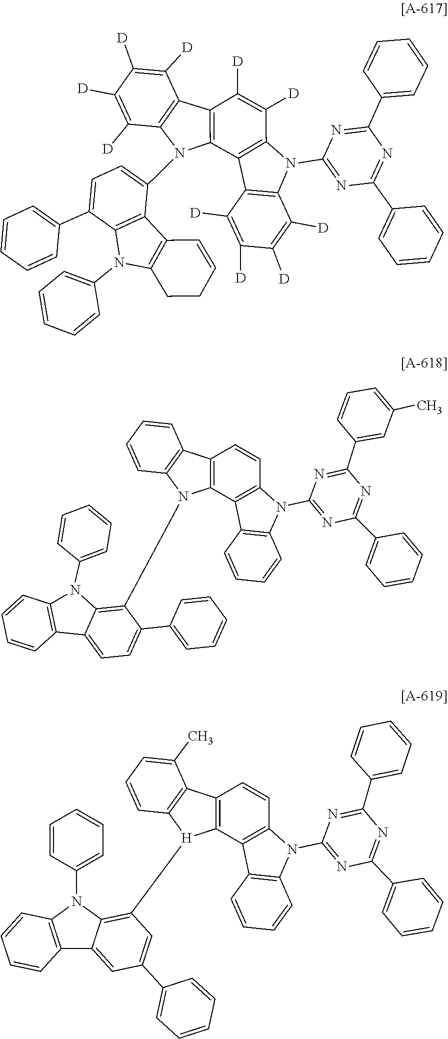

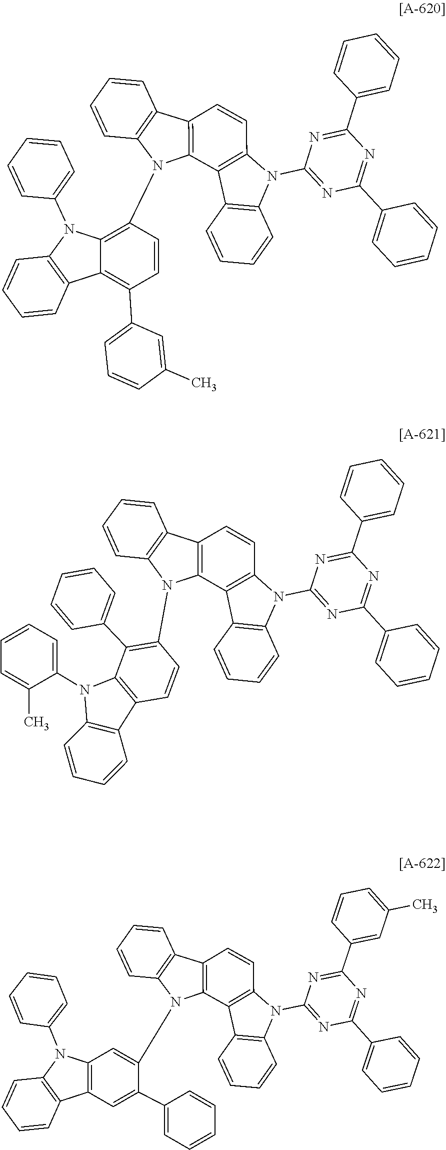 [SMILES: Cc1cccc(-c2ccc(-n3c4ccccc4c4ccc5c(c6ccccc6n5-c5nc(-c6ccccc6)nc(-c6ccccc6)n5)c43)c3c2c2ccccc2n3-c2ccccc2)c1 ; Cc1ccccc1-n1c2ccccc2c2ccc(-n3c4ccccc4c4ccc5c(c6ccccc6n5-c5nc(-c6ccccc6)nc(-c6ccccc6)n5)c43)c(-c3ccccc3)c21 ; Cc1cccc(-c2nc(-c3ccccc3)nc(-n3c4ccccc4c4c3ccc3c5ccccc5n(-c5cc6c(cc5-c5ccccc5)c5ccccc5n6-c5ccccc5)c34)n2)c1]

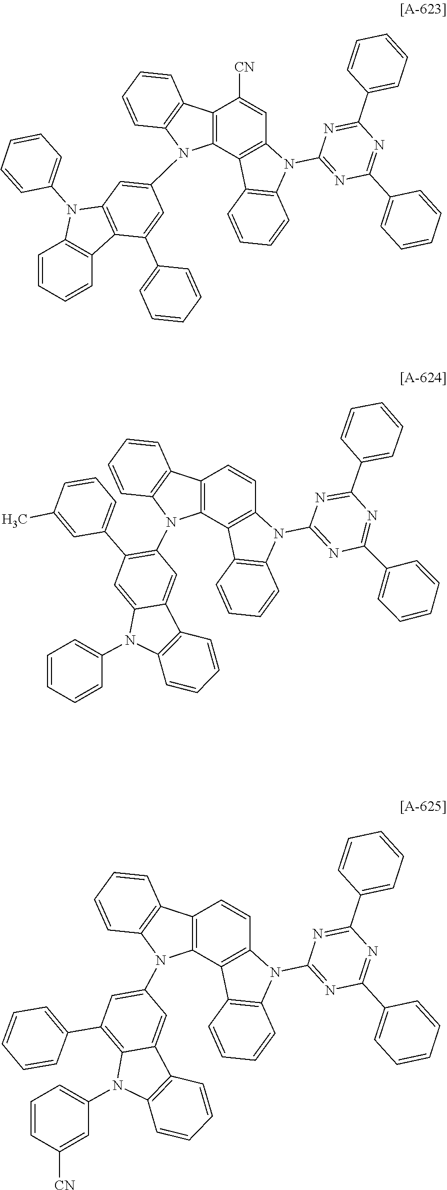 [SMILES: N#Cc1cc2c(c3ccccc3n2-c2nc(-c3ccccc3)nc(-c3ccccc3)n2)c2c1c1ccccc1n2-c1cc(-c2ccccc2)c2c3ccccc3n(-c3ccccc3)c2c1 ; Cc1cccc(-c2cc3c(cc2-n2c4ccccc4c4ccc5c(c6ccccc6n5-c5nc(-c6ccccc6)nc(-c6ccccc6)n5)c42)c2ccccc2n3-c2ccccc2)c1 ; N#Cc1cccc(-n2c3ccccc3c3cc(-n4c5ccccc5c5ccc6c(c7ccccc7n6-c6nc(-c7ccccc7)nc(-c7ccccc7)n6)c54)cc(-c4ccccc4)c32)c1]

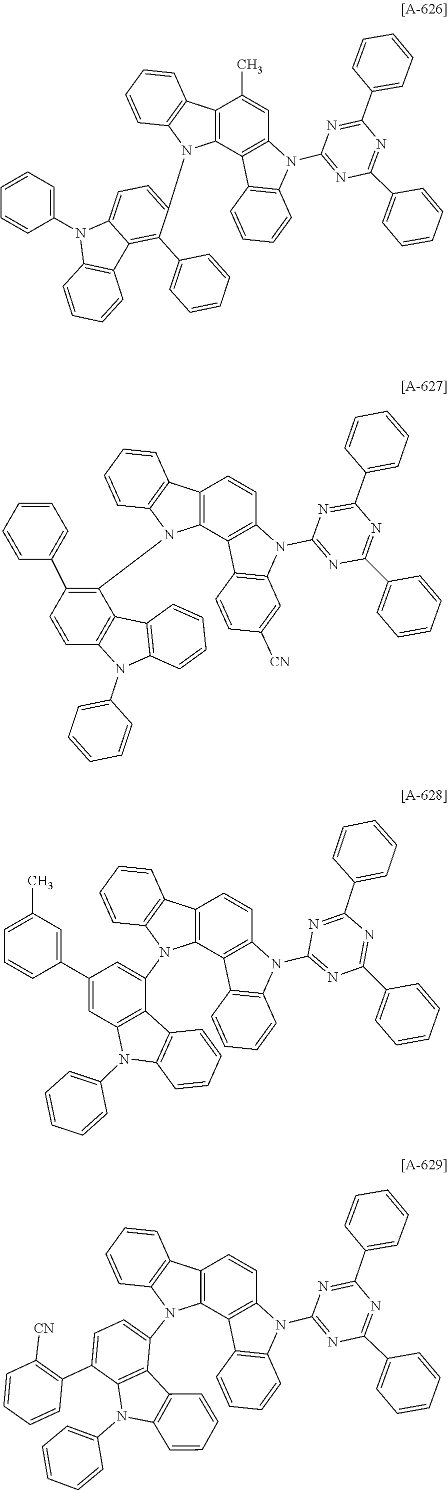 [SMILES: Cc1cc2c(c3ccccc3n2-c2nc(-c3ccccc3)nc(-c3ccccc3)n2)c2c1c1ccccc1n2-c1ccc2c(c1-c1ccccc1)c1ccccc1n2-c1ccccc1 ; N#Cc1ccc2c3c(ccc4c5ccccc5n(-c5c(-c6ccccc6)ccc6c5c5ccccc5n6-c5ccccc5)c43)n(-c3nc(-c4ccccc4)nc(-c4ccccc4)n3)c2c1 ; Cc1cccc(-c2cc(-n3c4ccccc4c4ccc5c(c6ccccc6n5-c5nc(-c6ccccc6)nc(-c6ccccc6)n5)c43)c3c4ccccc4n(-c4ccccc4)c3c2)c1 ; N#Cc1ccccc1-c1ccc(-n2c3ccccc3c3ccc4c(c5ccccc5n4-c4nc(-c5ccccc5)nc(-c5ccccc5)n4)c32)c2c3ccccc3n(-c3ccccc3)c12]

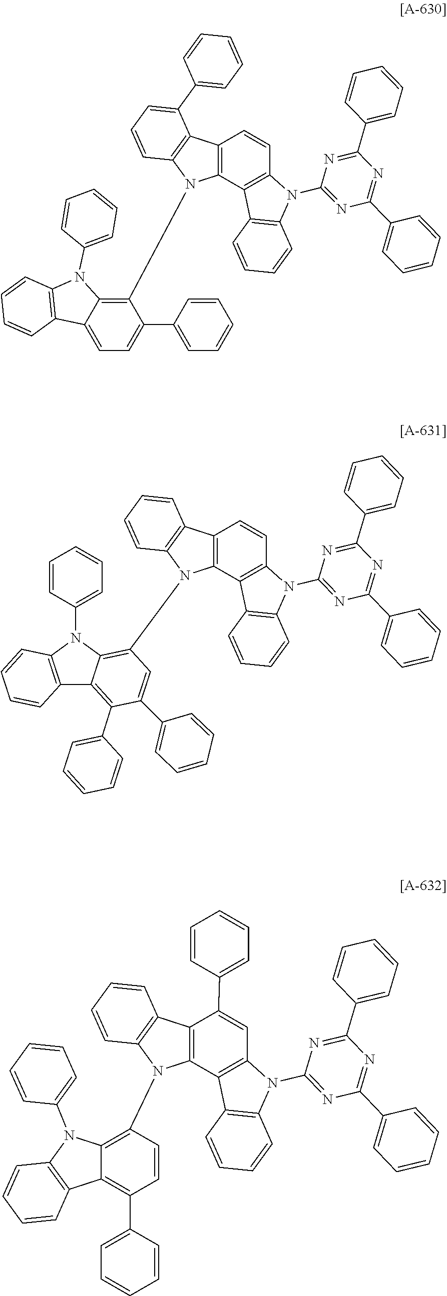 [SMILES: c1ccc(-c2nc(-c3ccccc3)nc(-n3c4ccccc4c4c3ccc3c5c(-c6ccccc6)cccc5n(-c5c(-c6ccccc6)ccc6c7ccccc7n(-c7ccccc7)c56)c34)n2)cc1 ; c1ccc(-c2nc(-c3ccccc3)nc(-n3c4ccccc4c4c3ccc3c5ccccc5n(-c5cc(-c6ccccc6)c(-c6ccccc6)c6c7ccccc7n(-c7ccccc7)c56)c34)n2)cc1 ; c1ccc(-c2nc(-c3ccccc3)nc(-n3c4ccccc4c4c3cc(-c3ccccc3)c3c5ccccc5n(-c5ccc(-c6ccccc6)c6c7ccccc7n(-c7ccccc7)c56)c34)n2)cc1]

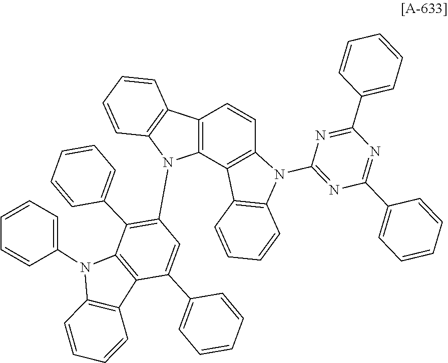 [SMILES: c1ccc(-c2nc(-c3ccccc3)nc(-n3c4ccccc4c4c3ccc3c5ccccc5n(-c5cc(-c6ccccc6)c6c7ccccc7n(-c7ccccc7)c6c5-c5ccccc5)c34)n2)cc1]

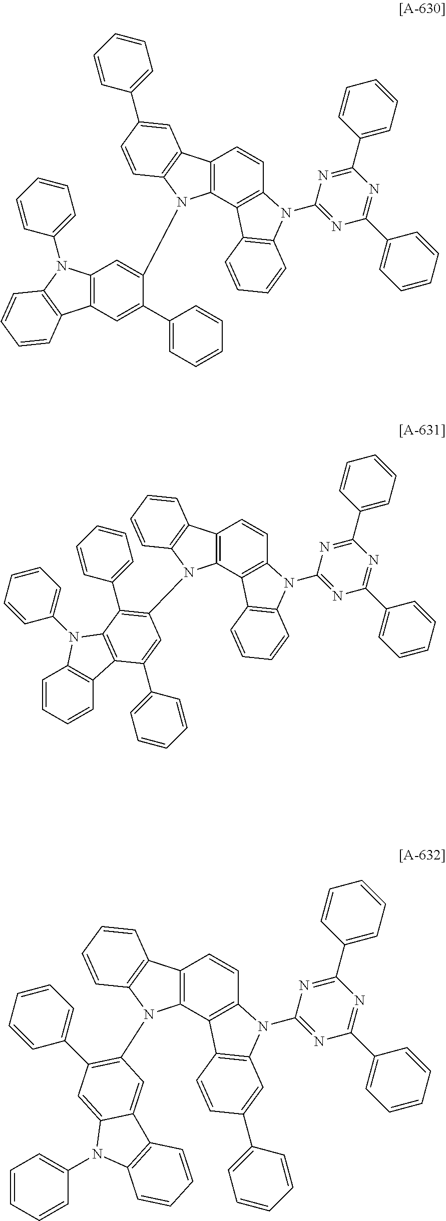 [SMILES: c1ccc(-c2ccc3c(c2)c2ccc4c(c5ccccc5n4-c4nc(-c5ccccc5)nc(-c5ccccc5)n4)c2n3-c2cc3c(cc2-c2ccccc2)c2ccccc2n3-c2ccccc2)cc1 ; c1ccc(-c2nc(-c3ccccc3)nc(-n3c4ccccc4c4c3ccc3c5ccccc5n(-c5cc(-c6ccccc6)c6c7ccccc7n(-c7ccccc7)c6c5-c5ccccc5)c34)n2)cc1 ; c1ccc(-c2ccc3c4c(ccc5c6ccccc6n(-c6cc7c8ccccc8n(-c8ccccc8)c7cc6-c6ccccc6)c54)n(-c4nc(-c5ccccc5)nc(-c5ccccc5)n4)c3c2)cc1]

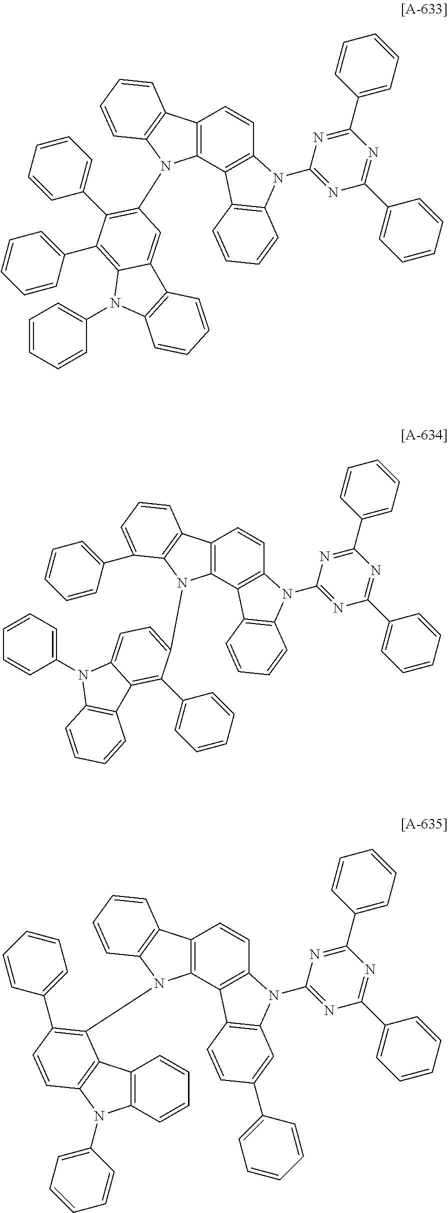 [SMILES: c1ccc(-c2nc(-c3ccccc3)nc(-n3c4ccccc4c4c3ccc3c5ccccc5n(-c5cc6c7ccccc7n(-c7ccccc7)c6c(-c6ccccc6)c5-c5ccccc5)c34)n2)cc1 ; c1ccc(-c2nc(-c3ccccc3)nc(-n3c4ccccc4c4c3ccc3c5cccc(-c6ccccc6)c5n(-c5ccc6c(c5-c5ccccc5)c5ccccc5n6-c5ccccc5)c34)n2)cc1 ; c1ccc(-c2ccc3c4c(ccc5c6ccccc6n(-c6c(-c7ccccc7)ccc7c6c6ccccc6n7-c6ccccc6)c54)n(-c4nc(-c5ccccc5)nc(-c5ccccc5)n4)c3c2)cc1]

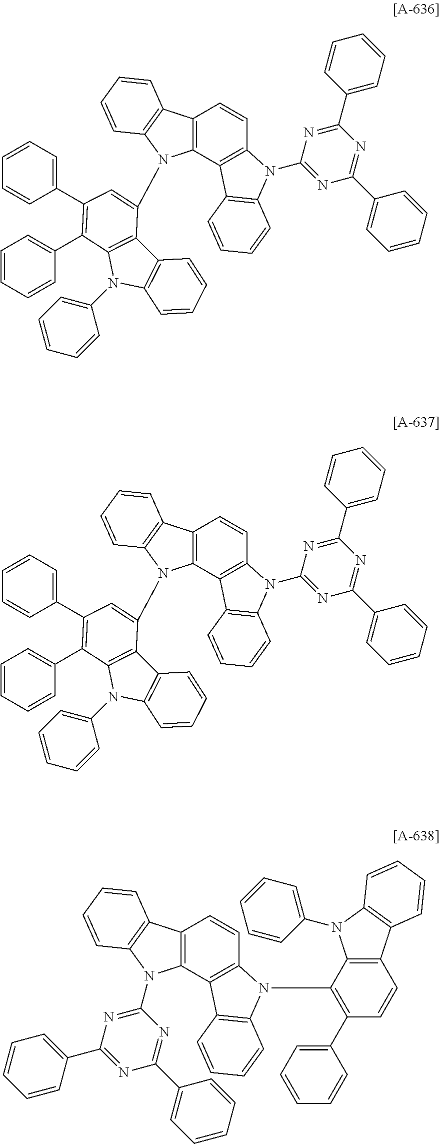 [SMILES: c1ccc(-c2nc(-c3ccccc3)nc(-n3c4ccccc4c4c3ccc3c5ccccc5n(-c5cc(-c6ccccc6)c(-c6ccccc6)c6c5c5ccccc5n6-c5ccccc5)c34)n2)cc1 ; c1ccc(-c2nc(-c3ccccc3)nc(-n3c4ccccc4c4c3ccc3c5ccccc5n(-c5cc(-c6ccccc6)c(-c6ccccc6)c6c5c5ccccc5n6-c5ccccc5)c34)n2)cc1 ; c1ccc(-c2nc(-c3ccccc3)nc(-n3c4ccccc4c4ccc5c(c6ccccc6n5-c5c(-c6ccccc6)ccc6c7ccccc7n(-c7ccccc7)c56)c43)n2)cc1]

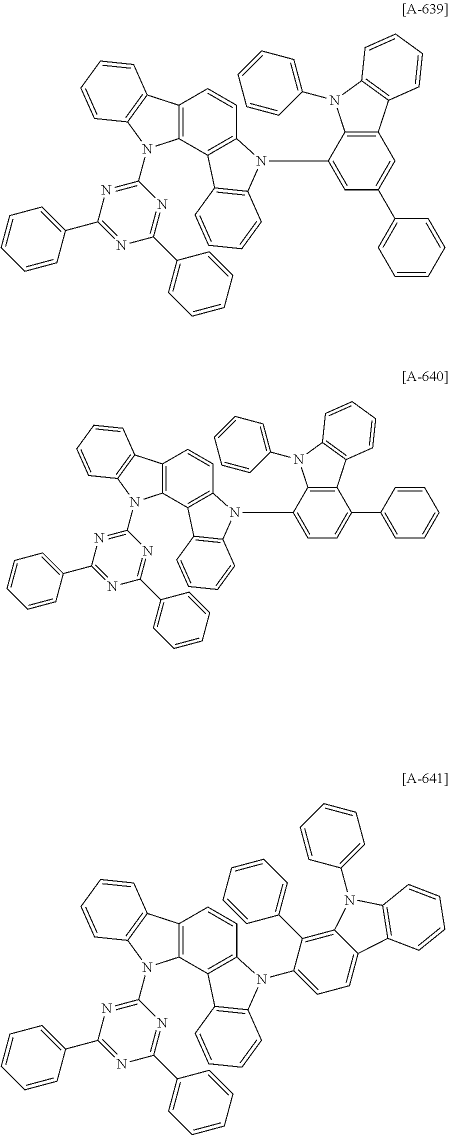 [SMILES: c1ccc(-c2cc(-n3c4ccccc4c4c3ccc3c5ccccc5n(-c5nc(-c6ccccc6)nc(-c6ccccc6)n5)c34)c3c(c2)c2ccccc2n3-c2ccccc2)cc1 ; c1ccc(-c2nc(-c3ccccc3)nc(-n3c4ccccc4c4ccc5c(c6ccccc6n5-c5ccc(-c6ccccc6)c6c7ccccc7n(-c7ccccc7)c56)c43)n2)cc1 ; c1ccc(-c2nc(-c3ccccc3)nc(-n3c4ccccc4c4ccc5c(c6ccccc6n5-c5ccc6c7ccccc7n(-c7ccccc7)c6c5-c5ccccc5)c43)n2)cc1]

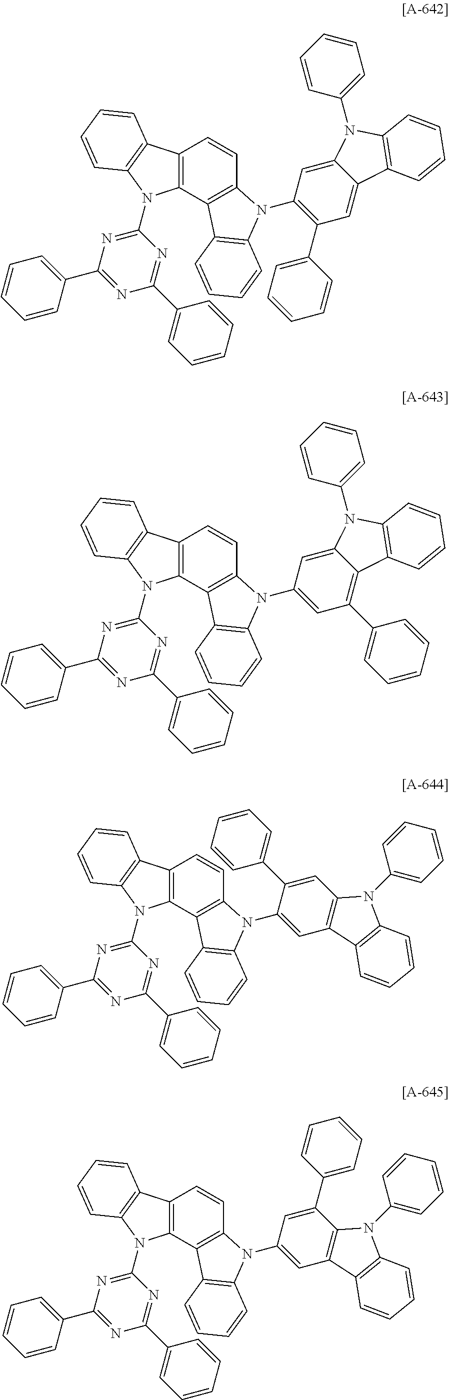 [SMILES: c1ccc(-c2nc(-c3ccccc3)nc(-n3c4ccccc4c4ccc5c(c6ccccc6n5-c5cc6c(cc5-c5ccccc5)c5ccccc5n6-c5ccccc5)c43)n2)cc1 ; c1ccc(-c2nc(-c3ccccc3)nc(-n3c4ccccc4c4ccc5c(c6ccccc6n5-c5cc(-c6ccccc6)c6c7ccccc7n(-c7ccccc7)c6c5)c43)n2)cc1 ; c1ccc(-c2nc(-c3ccccc3)nc(-n3c4ccccc4c4ccc5c(c6ccccc6n5-c5cc6c7ccccc7n(-c7ccccc7)c6cc5-c5ccccc5)c43)n2)cc1 ; c1ccc(-c2nc(-c3ccccc3)nc(-n3c4ccccc4c4ccc5c(c6ccccc6n5-c5cc(-c6ccccc6)c6c(c5)c5ccccc5n6-c5ccccc5)c43)n2)cc1]

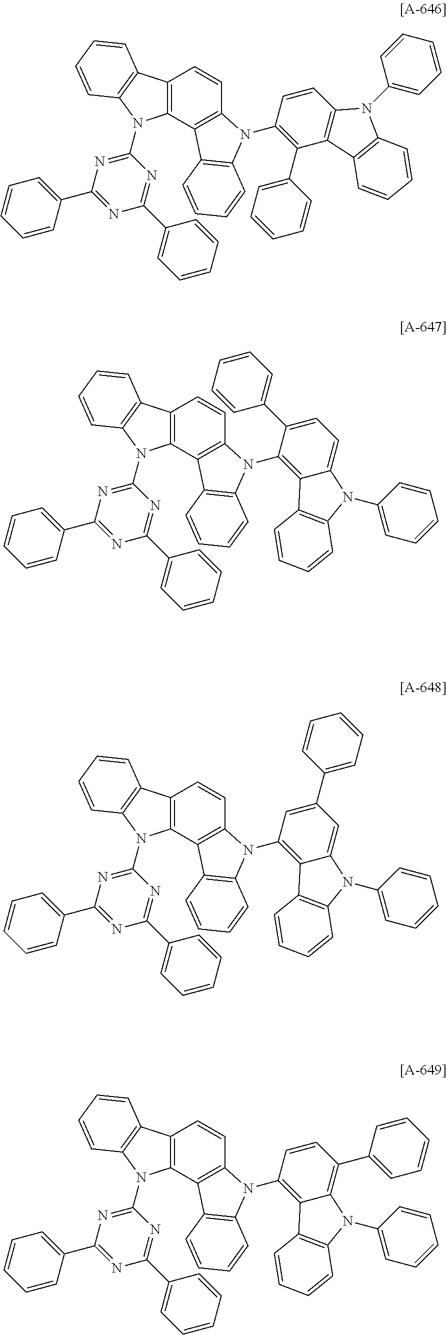 [SMILES: c1ccc(-c2nc(-c3ccccc3)nc(-n3c4ccccc4c4ccc5c(c6ccccc6n5-c5ccc6c(c5-c5ccccc5)c5ccccc5n6-c5ccccc5)c43)n2)cc1 ; c1ccc(-c2nc(-c3ccccc3)nc(-n3c4ccccc4c4ccc5c(c6ccccc6n5-c5c(-c6ccccc6)ccc6c5c5ccccc5n6-c5ccccc5)c43)n2)cc1 ; c1ccc(-c2cc(-n3c4ccccc4c4c3ccc3c5ccccc5n(-c5nc(-c6ccccc6)nc(-c6ccccc6)n5)c34)c3c4ccccc4n(-c4ccccc4)c3c2)cc1 ; c1ccc(-c2nc(-c3ccccc3)nc(-n3c4ccccc4c4ccc5c(c6ccccc6n5-c5ccc(-c6ccccc6)c6c5c5ccccc5n6-c5ccccc5)c43)n2)cc1]

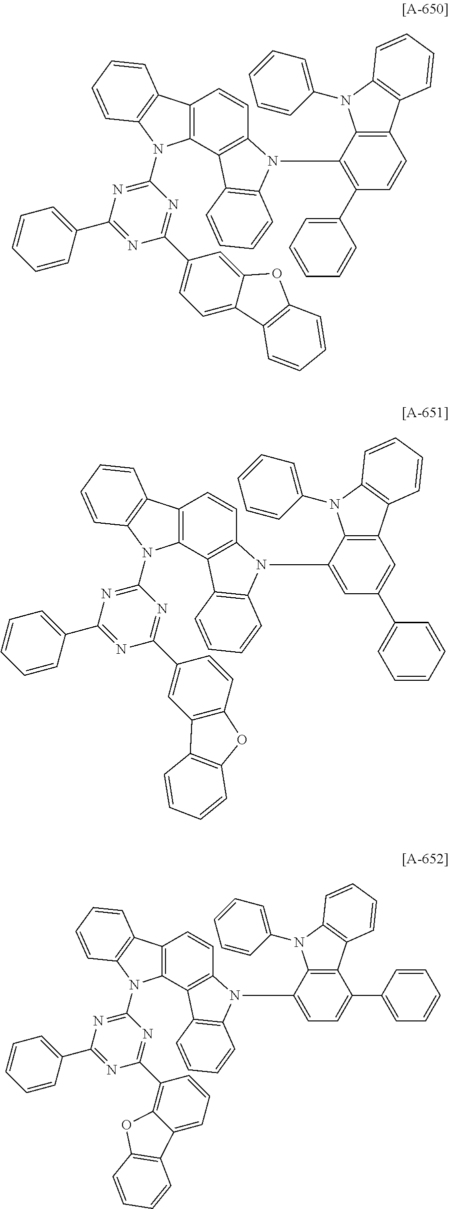 [SMILES: c1ccc(-c2nc(-c3ccc4c(c3)oc3ccccc34)nc(-n3c4ccccc4c4ccc5c(c6ccccc6n5-c5c(-c6ccccc6)ccc6c7ccccc7n(-c7ccccc7)c56)c43)n2)cc1 ; c1ccc(-c2cc(-n3c4ccccc4c4c3ccc3c5ccccc5n(-c5nc(-c6ccccc6)nc(-c6ccc7oc8ccccc8c7c6)n5)c34)c3c(c2)c2ccccc2n3-c2ccccc2)cc1 ; c1ccc(-c2nc(-c3cccc4c3oc3ccccc34)nc(-n3c4ccccc4c4ccc5c(c6ccccc6n5-c5ccc(-c6ccccc6)c6c7ccccc7n(-c7ccccc7)c56)c43)n2)cc1]

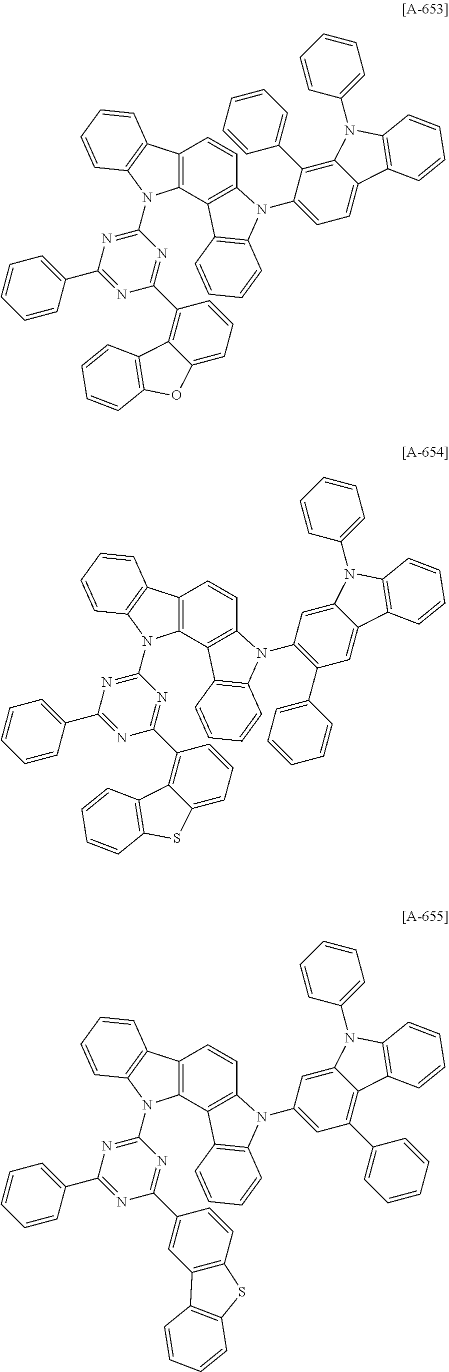 [SMILES: c1ccc(-c2nc(-c3cccc4oc5ccccc5c34)nc(-n3c4ccccc4c4ccc5c(c6ccccc6n5-c5ccc6c7ccccc7n(-c7ccccc7)c6c5-c5ccccc5)c43)n2)cc1 ; c1ccc(-c2nc(-c3cccc4sc5ccccc5c34)nc(-n3c4ccccc4c4ccc5c(c6ccccc6n5-c5cc6c(cc5-c5ccccc5)c5ccccc5n6-c5ccccc5)c43)n2)cc1 ; c1ccc(-c2nc(-c3ccc4sc5ccccc5c4c3)nc(-n3c4ccccc4c4ccc5c(c6ccccc6n5-c5cc(-c6ccccc6)c6c7ccccc7n(-c7ccccc7)c6c5)c43)n2)cc1]

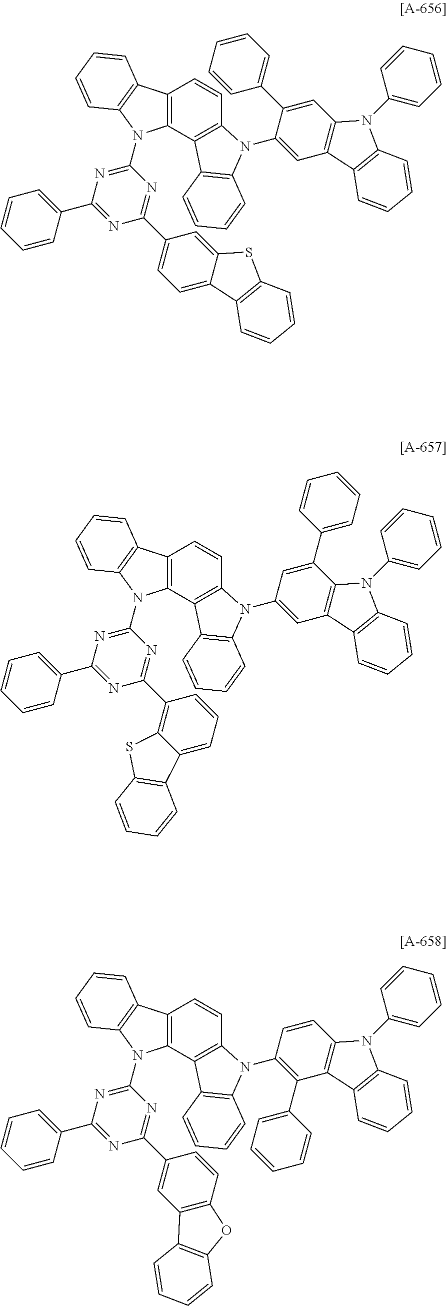 [SMILES: c1ccc(-c2nc(-c3ccc4c(c3)sc3ccccc34)nc(-n3c4ccccc4c4ccc5c(c6ccccc6n5-c5cc6c7ccccc7n(-c7ccccc7)c6cc5-c5ccccc5)c43)n2)cc1 ; c1ccc(-c2nc(-c3cccc4c3sc3ccccc34)nc(-n3c4ccccc4c4ccc5c(c6ccccc6n5-c5cc(-c6ccccc6)c6c(c5)c5ccccc5n6-c5ccccc5)c43)n2)cc1 ; c1ccc(-c2nc(-c3ccc4oc5ccccc5c4c3)nc(-n3c4ccccc4c4ccc5c(c6ccccc6n5-c5ccc6c(c5-c5ccccc5)c5ccccc5n6-c5ccccc5)c43)n2)cc1]

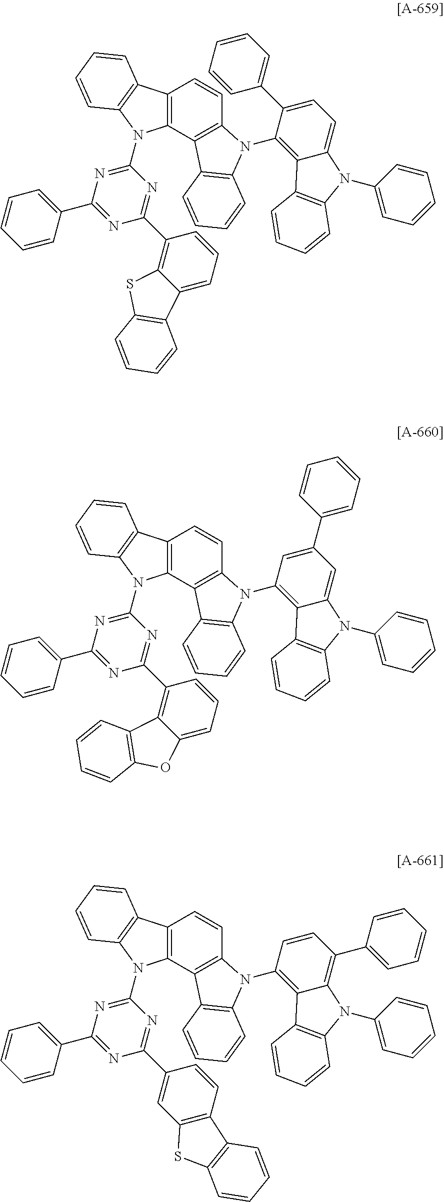 [SMILES: c1ccc(-c2nc(-c3cccc4c3sc3ccccc34)nc(-n3c4ccccc4c4ccc5c(c6ccccc6n5-c5c(-c6ccccc6)ccc6c5c5ccccc5n6-c5ccccc5)c43)n2)cc1 ; c1ccc(-c2cc(-n3c4ccccc4c4c3ccc3c5ccccc5n(-c5nc(-c6ccccc6)nc(-c6cccc7oc8ccccc8c67)n5)c34)c3c4ccccc4n(-c4ccccc4)c3c2)cc1 ; c1ccc(-c2nc(-c3ccc4c(c3)sc3ccccc34)nc(-n3c4ccccc4c4ccc5c(c6ccccc6n5-c5ccc(-c6ccccc6)c6c5c5ccccc5n6-c5ccccc5)c43)n2)cc1]

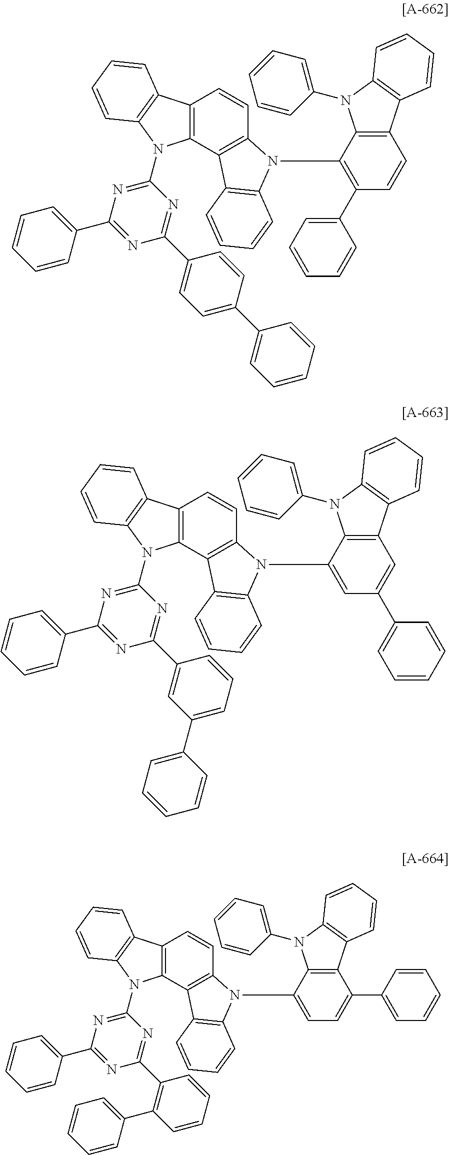 [SMILES: c1ccc(-c2ccc(-c3nc(-c4ccccc4)nc(-n4c5ccccc5c5ccc6c(c7ccccc7n6-c6c(-c7ccccc7)ccc7c8ccccc8n(-c8ccccc8)c67)c54)n3)cc2)cc1 ; c1ccc(-c2cccc(-c3nc(-c4ccccc4)nc(-n4c5ccccc5c5ccc6c(c7ccccc7n6-c6cc(-c7ccccc7)cc7c8ccccc8n(-c8ccccc8)c67)c54)n3)c2)cc1 ; c1ccc(-c2nc(-c3ccccc3-c3ccccc3)nc(-n3c4ccccc4c4ccc5c(c6ccccc6n5-c5ccc(-c6ccccc6)c6c7ccccc7n(-c7ccccc7)c56)c43)n2)cc1]

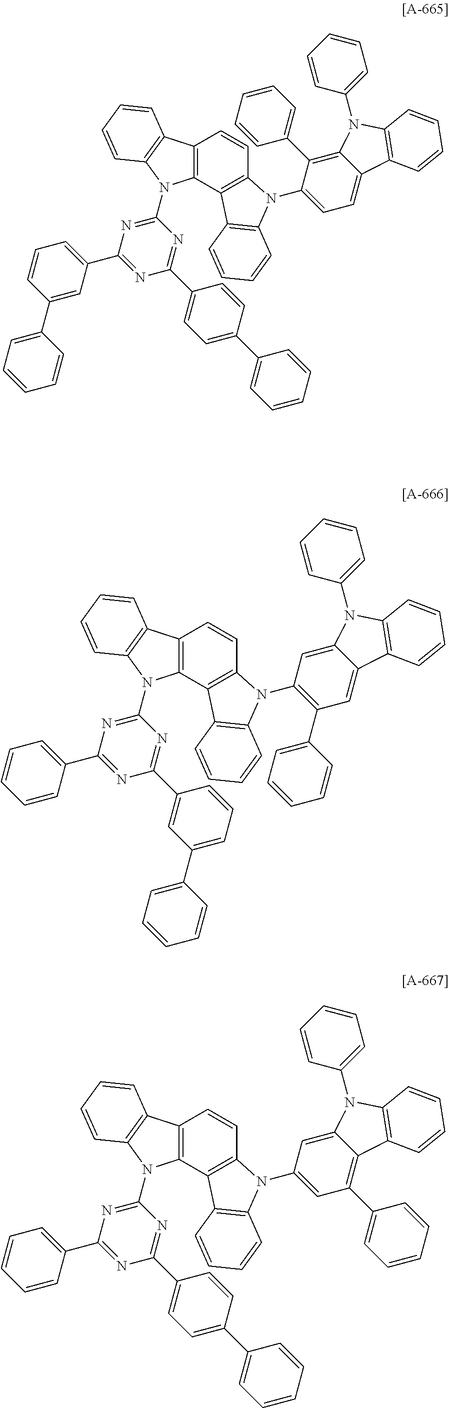 [SMILES: c1ccc(-c2ccc(-c3nc(-c4cccc(-c5ccccc5)c4)nc(-n4c5ccccc5c5ccc6c(c7ccccc7n6-c6ccc7c8ccccc8n(-c8ccccc8)c7c6-c6ccccc6)c54)n3)cc2)cc1 ; c1ccc(-c2cccc(-c3nc(-c4ccccc4)nc(-n4c5ccccc5c5ccc6c(c7ccccc7n6-c6cc7c(cc6-c6ccccc6)c6ccccc6n7-c6ccccc6)c54)n3)c2)cc1 ; c1ccc(-c2ccc(-c3nc(-c4ccccc4)nc(-n4c5ccccc5c5ccc6c(c7ccccc7n6-c6cc(-c7ccccc7)c7c8ccccc8n(-c8ccccc8)c7c6)c54)n3)cc2)cc1]

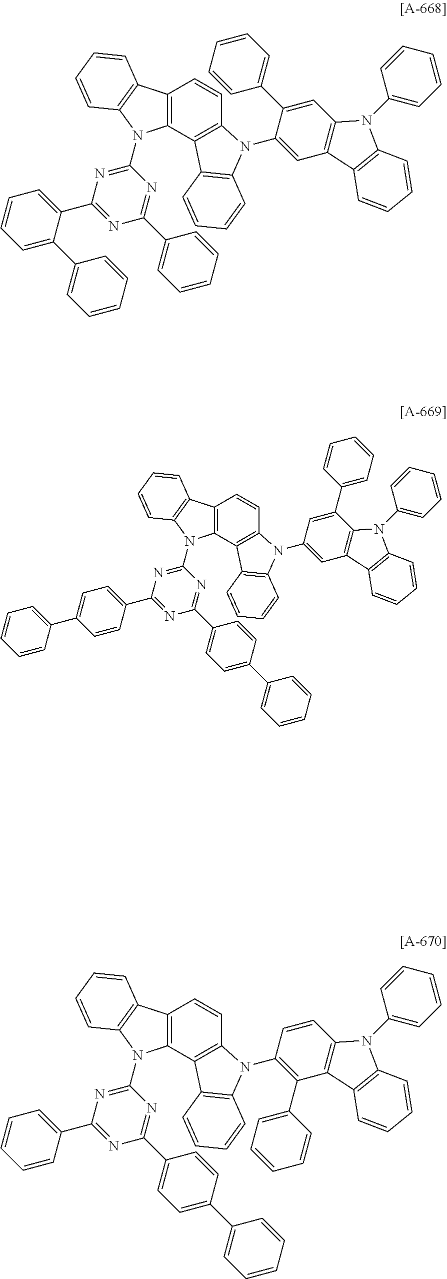 [SMILES: c1ccc(-c2nc(-c3ccccc3-c3ccccc3)nc(-n3c4ccccc4c4ccc5c(c6ccccc6n5-c5cc6c7ccccc7n(-c7ccccc7)c6cc5-c5ccccc5)c43)n2)cc1 ; c1ccc(-c2ccc(-c3nc(-c4ccc(-c5ccccc5)cc4)nc(-n4c5ccccc5c5ccc6c(c7ccccc7n6-c6cc(-c7ccccc7)c7c(c6)c6ccccc6n7-c6ccccc6)c54)n3)cc2)cc1 ; c1ccc(-c2ccc(-c3nc(-c4ccccc4)nc(-n4c5ccccc5c5ccc6c(c7ccccc7n6-c6ccc7c(c6-c6ccccc6)c6ccccc6n7-c6ccccc6)c54)n3)cc2)cc1]

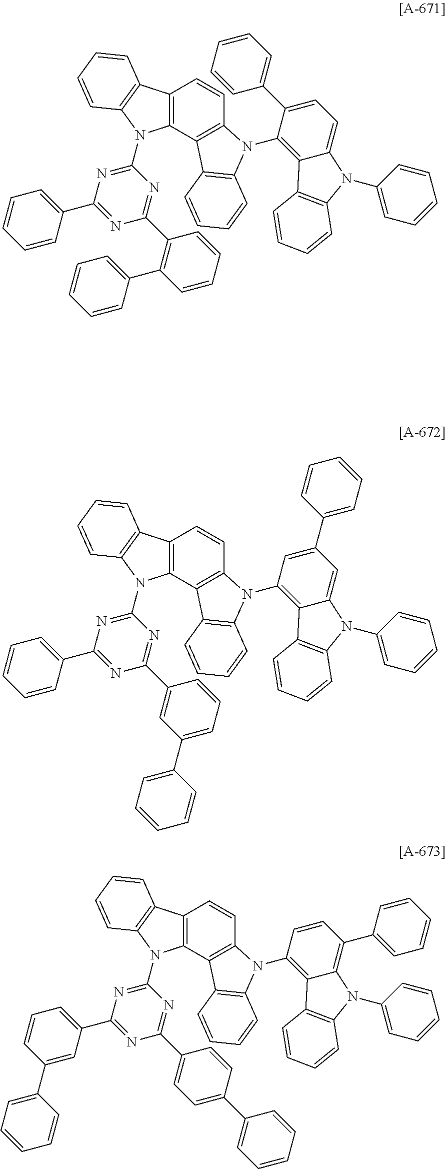 [SMILES: c1ccc(-c2nc(-c3ccccc3-c3ccccc3)nc(-n3c4ccccc4c4ccc5c(c6ccccc6n5-c5c(-c6ccccc6)ccc6c5c5ccccc5n6-c5ccccc5)c43)n2)cc1 ; c1ccc(-c2cccc(-c3nc(-c4ccccc4)nc(-n4c5ccccc5c5ccc6c(c7ccccc7n6-c6cc(-c7ccccc7)cc7c6c6ccccc6n7-c6ccccc6)c54)n3)c2)cc1 ; c1ccc(-c2ccc(-c3nc(-c4cccc(-c5ccccc5)c4)nc(-n4c5ccccc5c5ccc6c(c7ccccc7n6-c6ccc(-c7ccccc7)c7c6c6ccccc6n7-c6ccccc6)c54)n3)cc2)cc1]

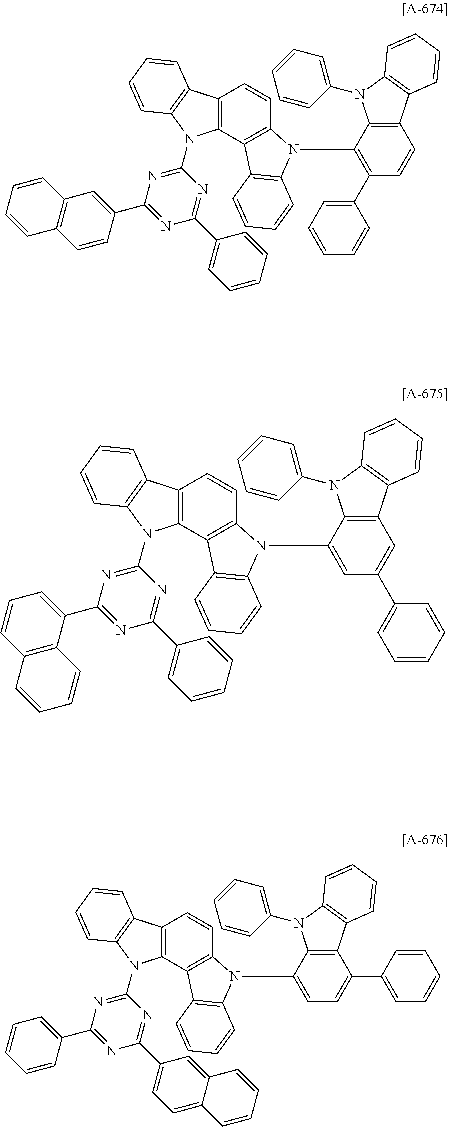 [SMILES: c1ccc(-c2nc(-c3ccc4ccccc4c3)nc(-n3c4ccccc4c4ccc5c(c6ccccc6n5-c5c(-c6ccccc6)ccc6c7ccccc7n(-c7ccccc7)c56)c43)n2)cc1 ; c1ccc(-c2cc(-n3c4ccccc4c4c3ccc3c5ccccc5n(-c5nc(-c6ccccc6)nc(-c6cccc7ccccc67)n5)c34)c3c(c2)c2ccccc2n3-c2ccccc2)cc1 ; c1ccc(-c2nc(-c3ccc4ccccc4c3)nc(-n3c4ccccc4c4ccc5c(c6ccccc6n5-c5ccc(-c6ccccc6)c6c7ccccc7n(-c7ccccc7)c56)c43)n2)cc1]

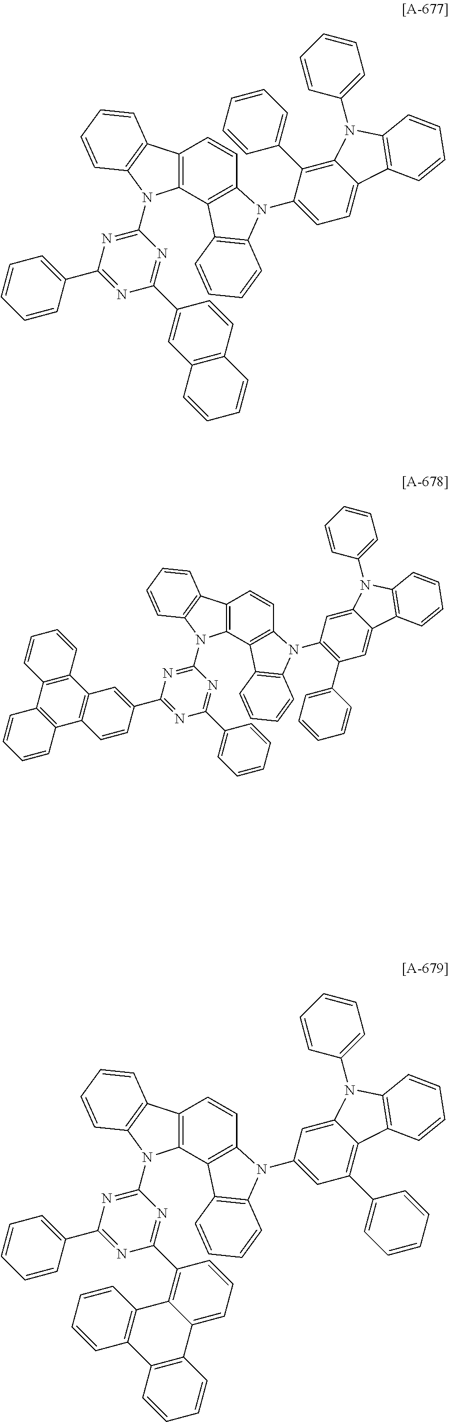 [SMILES: c1ccc(-c2nc(-c3ccc4ccccc4c3)nc(-n3c4ccccc4c4ccc5c(c6ccccc6n5-c5ccc6c7ccccc7n(-c7ccccc7)c6c5-c5ccccc5)c43)n2)cc1 ; c1ccc(-c2nc(-c3ccc4c5ccccc5c5ccccc5c4c3)nc(-n3c4ccccc4c4ccc5c(c6ccccc6n5-c5cc6c(cc5-c5ccccc5)c5ccccc5n6-c5ccccc5)c43)n2)cc1 ; c1ccc(-c2nc(-c3cccc4c5ccccc5c5ccccc5c34)nc(-n3c4ccccc4c4ccc5c(c6ccccc6n5-c5cc(-c6ccccc6)c6c7ccccc7n(-c7ccccc7)c6c5)c43)n2)cc1]

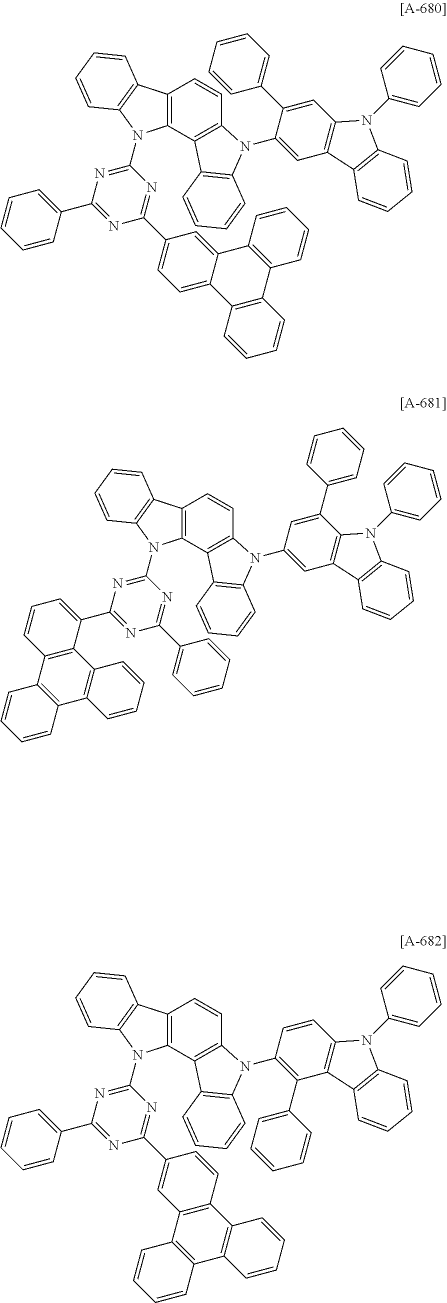 [SMILES: c1ccc(-c2nc(-c3ccc4c5ccccc5c5ccccc5c4c3)nc(-n3c4ccccc4c4ccc5c(c6ccccc6n5-c5cc6c7ccccc7n(-c7ccccc7)c6cc5-c5ccccc5)c43)n2)cc1 ; c1ccc(-c2nc(-c3cccc4c5ccccc5c5ccccc5c34)nc(-n3c4ccccc4c4ccc5c(c6ccccc6n5-c5cc(-c6ccccc6)c6c(c5)c5ccccc5n6-c5ccccc5)c43)n2)cc1 ; c1ccc(-c2nc(-c3ccc4c5ccccc5c5ccccc5c4c3)nc(-n3c4ccccc4c4ccc5c(c6ccccc6n5-c5ccc6c(c5-c5ccccc5)c5ccccc5n6-c5ccccc5)c43)n2)cc1]

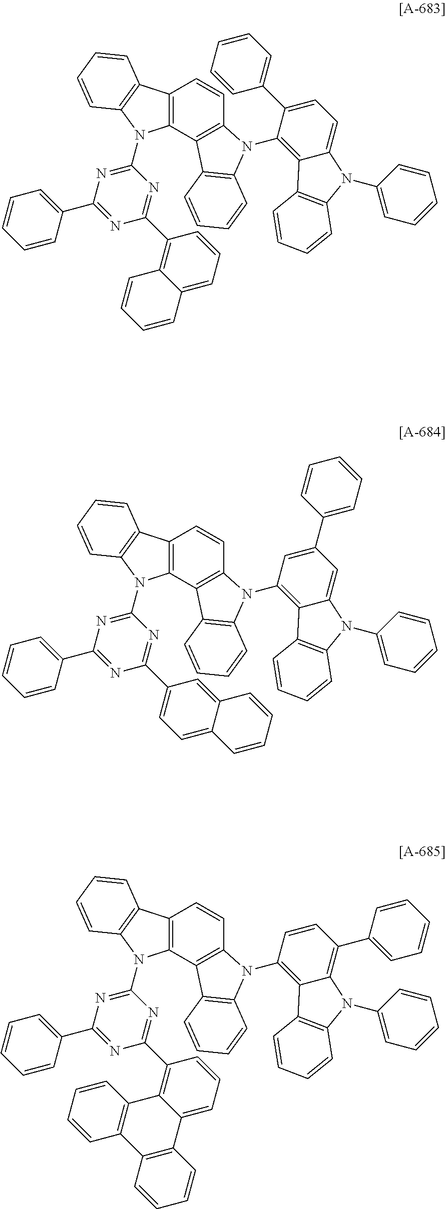 [SMILES: c1ccc(-c2nc(-c3cccc4ccccc34)nc(-n3c4ccccc4c4ccc5c(c6ccccc6n5-c5c(-c6ccccc6)ccc6c5c5ccccc5n6-c5ccccc5)c43)n2)cc1 ; c1ccc(-c2cc(-n3c4ccccc4c4c3ccc3c5ccccc5n(-c5nc(-c6ccccc6)nc(-c6ccc7ccccc7c6)n5)c34)c3c4ccccc4n(-c4ccccc4)c3c2)cc1 ; c1ccc(-c2nc(-c3cccc4c5ccccc5c5ccccc5c34)nc(-n3c4ccccc4c4ccc5c(c6ccccc6n5-c5ccc(-c6ccccc6)c6c5c5ccccc5n6-c5ccccc5)c43)n2)cc1]

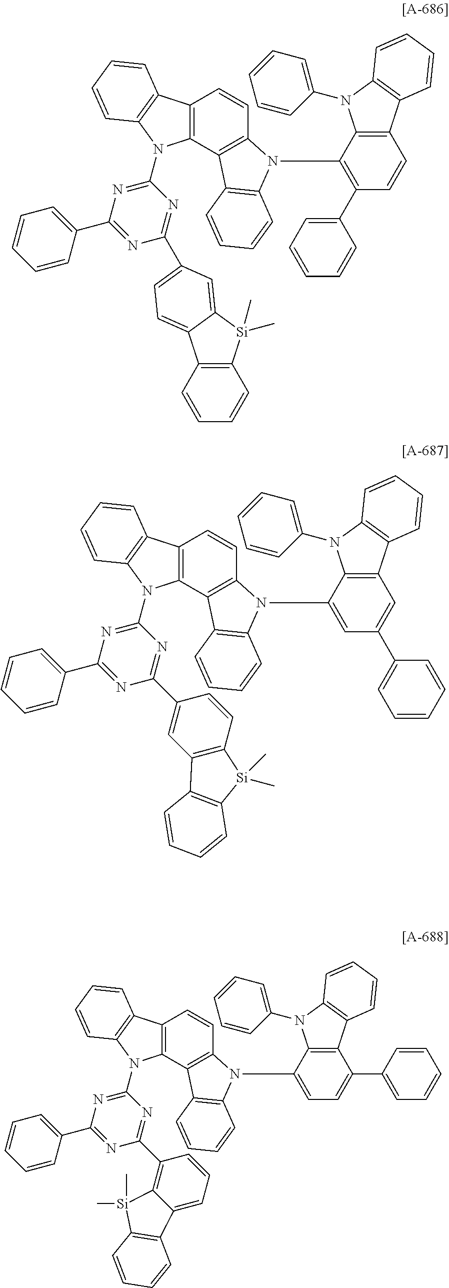 [SMILES: C[Si]1(C)c2ccccc2-c2ccc(-c3nc(-c4ccccc4)nc(-n4c5ccccc5c5ccc6c(c7ccccc7n6-c6c(-c7ccccc7)ccc7c8ccccc8n(-c8ccccc8)c67)c54)n3)cc21 ; C[Si]1(C)c2ccccc2-c2cc(-c3nc(-c4ccccc4)nc(-n4c5ccccc5c5ccc6c(c7ccccc7n6-c6cc(-c7ccccc7)cc7c8ccccc8n(-c8ccccc8)c67)c54)n3)ccc21 ; C[Si]1(C)c2ccccc2-c2cccc(-c3nc(-c4ccccc4)nc(-n4c5ccccc5c5ccc6c(c7ccccc7n6-c6ccc(-c7ccccc7)c7c8ccccc8n(-c8ccccc8)c67)c54)n3)c21]

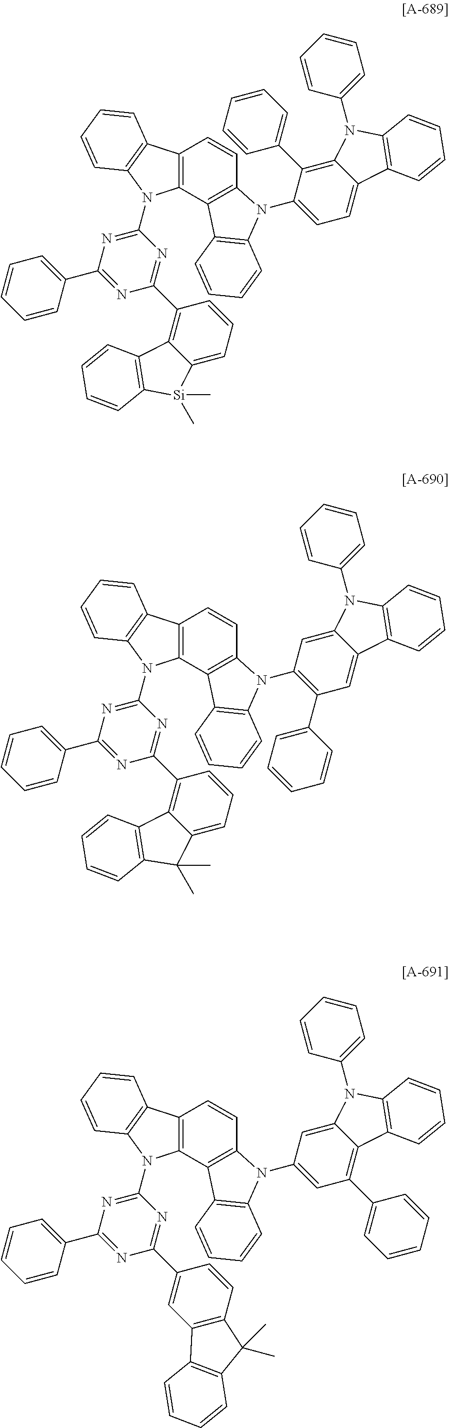 [SMILES: C[Si]1(C)c2ccccc2-c2c(-c3nc(-c4ccccc4)nc(-n4c5ccccc5c5ccc6c(c7ccccc7n6-c6ccc7c8ccccc8n(-c8ccccc8)c7c6-c6ccccc6)c54)n3)cccc21 ; CC1(C)c2ccccc2-c2c(-c3nc(-c4ccccc4)nc(-n4c5ccccc5c5ccc6c(c7ccccc7n6-c6cc7c(cc6-c6ccccc6)c6ccccc6n7-c6ccccc6)c54)n3)cccc21 ; CC1(C)c2ccccc2-c2cc(-c3nc(-c4ccccc4)nc(-n4c5ccccc5c5ccc6c(c7ccccc7n6-c6cc(-c7ccccc7)c7c8ccccc8n(-c8ccccc8)c7c6)c54)n3)ccc21]

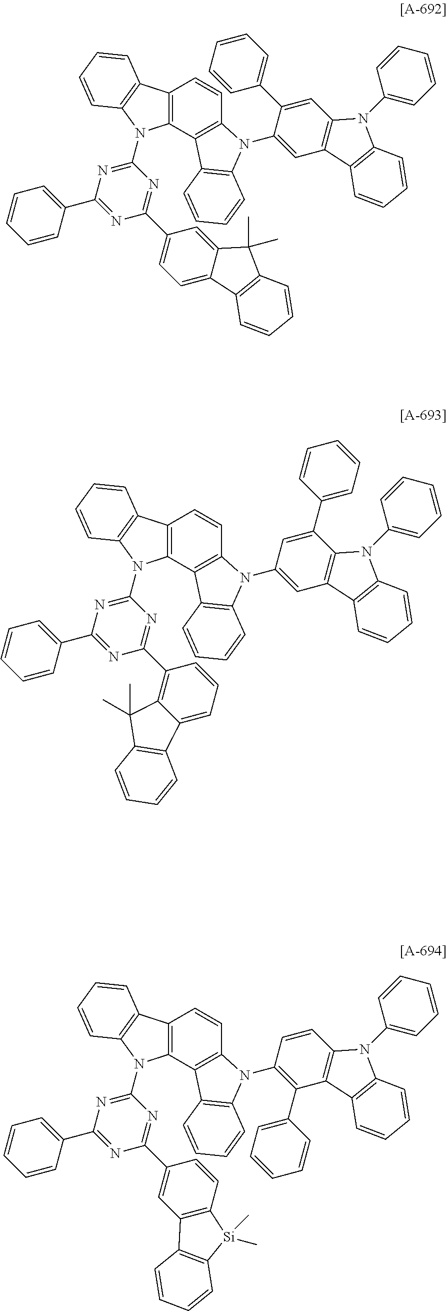 [SMILES: CC1(C)c2ccccc2-c2ccc(-c3nc(-c4ccccc4)nc(-n4c5ccccc5c5ccc6c(c7ccccc7n6-c6cc7c8ccccc8n(-c8ccccc8)c7cc6-c6ccccc6)c54)n3)cc21 ; CC1(C)c2ccccc2-c2cccc(-c3nc(-c4ccccc4)nc(-n4c5ccccc5c5ccc6c(c7ccccc7n6-c6cc(-c7ccccc7)c7c(c6)c6ccccc6n7-c6ccccc6)c54)n3)c21 ; C[Si]1(C)c2ccccc2-c2cc(-c3nc(-c4ccccc4)nc(-n4c5ccccc5c5ccc6c(c7ccccc7n6-c6ccc7c(c6-c6ccccc6)c6ccccc6n7-c6ccccc6)c54)n3)ccc21]

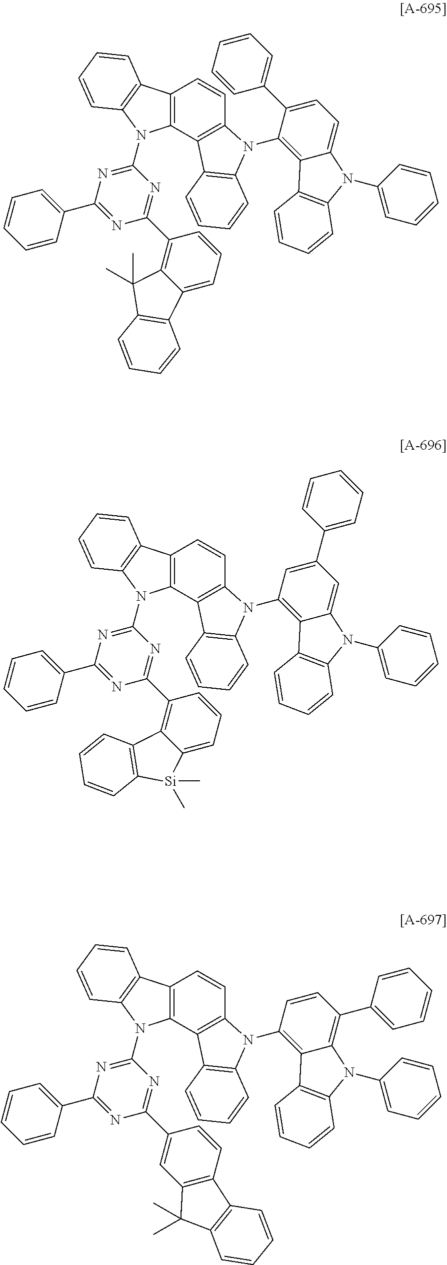 [SMILES: CC1(C)c2ccccc2-c2cccc(-c3nc(-c4ccccc4)nc(-n4c5ccccc5c5ccc6c(c7ccccc7n6-c6c(-c7ccccc7)ccc7c6c6ccccc6n7-c6ccccc6)c54)n3)c21 ; C[Si]1(C)c2ccccc2-c2c(-c3nc(-c4ccccc4)nc(-n4c5ccccc5c5ccc6c(c7ccccc7n6-c6cc(-c7ccccc7)cc7c6c6ccccc6n7-c6ccccc6)c54)n3)cccc21 ; CC1(C)c2ccccc2-c2ccc(-c3nc(-c4ccccc4)nc(-n4c5ccccc5c5ccc6c(c7ccccc7n6-c6ccc(-c7ccccc7)c7c6c6ccccc6n7-c6ccccc6)c54)n3)cc21]

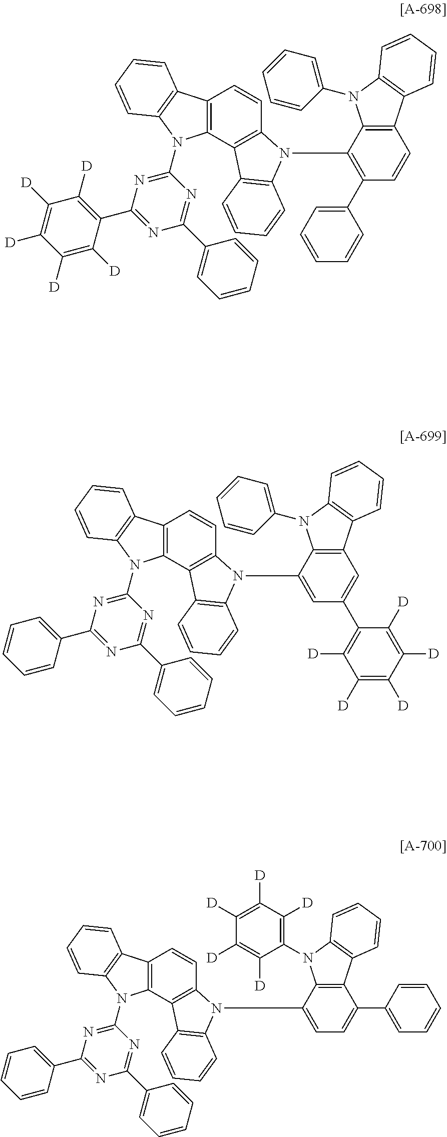 [SMILES: [2H]c1c([2H])c([2H])c(-c2nc(-c3ccccc3)nc(-n3c4ccccc4c4ccc5c(c6ccccc6n5-c5c(-c6ccccc6)ccc6c7ccccc7n(-c7ccccc7)c56)c43)n2)c([2H])c1[2H] ; [2H]c1c([2H])c([2H])c(-c2cc(-n3c4ccccc4c4c3ccc3c5ccccc5n(-c5nc(-c6ccccc6)nc(-c6ccccc6)n5)c34)c3c(c2)c2ccccc2n3-c2ccccc2)c([2H])c1[2H] ; [2H]c1c([2H])c([2H])c(-n2c3ccccc3c3c(-c4ccccc4)ccc(-n4c5ccccc5c5c4ccc4c6ccccc6n(-c6nc(-c7ccccc7)nc(-c7ccccc7)n6)c45)c32)c([2H])c1[2H]]

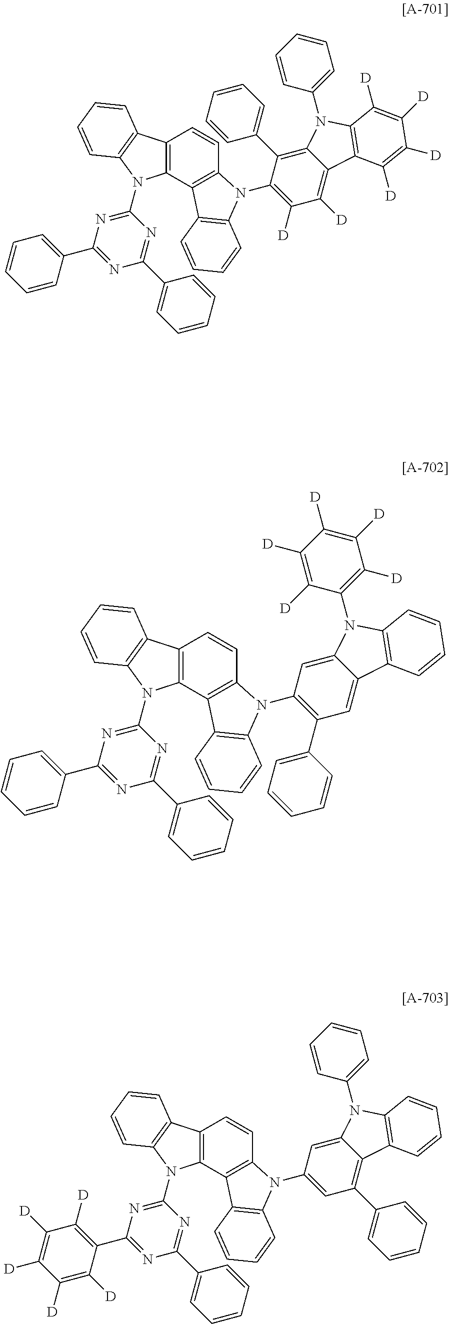 [SMILES: [2H]c1c([2H])c([2H])c2c(c1[2H])c1c([2H])c([2H])c(-n3c4ccccc4c4c3ccc3c5ccccc5n(-c5nc(-c6ccccc6)nc(-c6ccccc6)n5)c34)c(-c3ccccc3)c1n2-c1ccccc1 ; [2H]c1c([2H])c([2H])c(-n2c3ccccc3c3cc(-c4ccccc4)c(-n4c5ccccc5c5c4ccc4c6ccccc6n(-c6nc(-c7ccccc7)nc(-c7ccccc7)n6)c45)cc32)c([2H])c1[2H] ; [2H]c1c([2H])c([2H])c(-c2nc(-c3ccccc3)nc(-n3c4ccccc4c4ccc5c(c6ccccc6n5-c5cc(-c6ccccc6)c6c7ccccc7n(-c7ccccc7)c6c5)c43)n2)c([2H])c1[2H]]

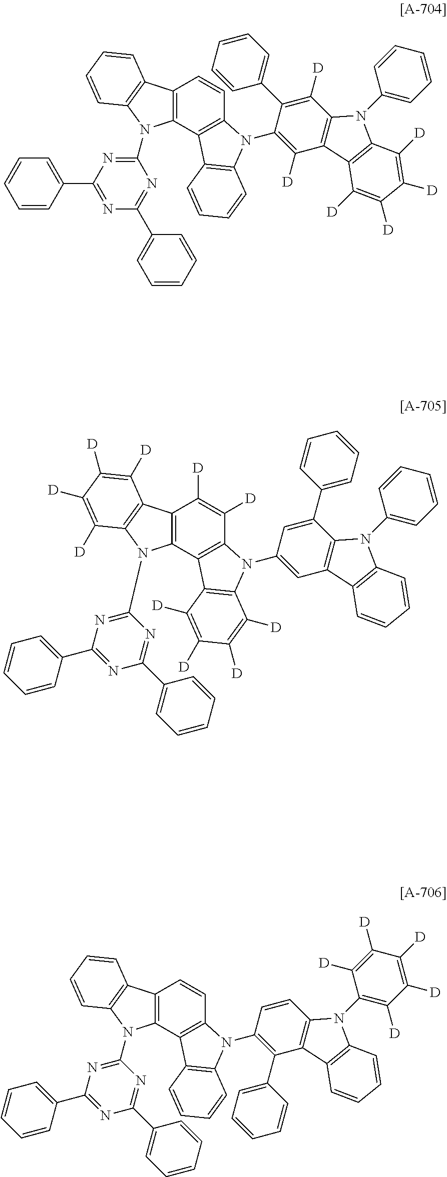 [SMILES: [2H]c1c([2H])c([2H])c2c(c1[2H])c1c([2H])c(-n3c4ccccc4c4c3ccc3c5ccccc5n(-c5nc(-c6ccccc6)nc(-c6ccccc6)n5)c34)c(-c3ccccc3)c([2H])c1n2-c1ccccc1 ; [2H]c1c([2H])c([2H])c2c(c1[2H])c1c(c([2H])c([2H])c3c4c([2H])c([2H])c([2H])c([2H])c4n(-c4nc(-c5ccccc5)nc(-c5ccccc5)n4)c31)n2-c1cc(-c2ccccc2)c2c(c1)c1ccccc1n2-c1ccccc1 ; [2H]c1c([2H])c([2H])c(-n2c3ccccc3c3c(-c4ccccc4)c(-n4c5ccccc5c5c4ccc4c6ccccc6n(-c6nc(-c7ccccc7)nc(-c7ccccc7)n6)c45)ccc32)c([2H])c1[2H]]

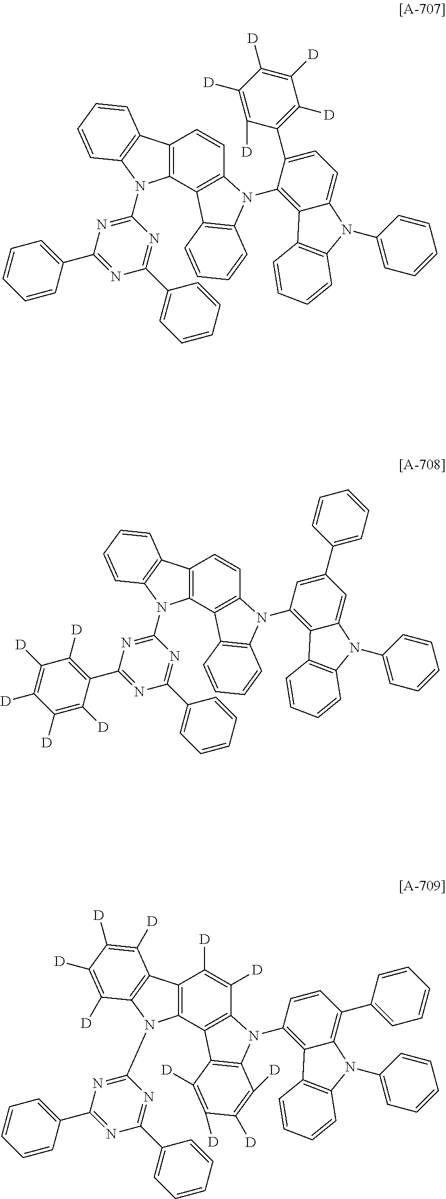 [SMILES: [2H]c1c([2H])c([2H])c(-c2ccc3c(c2-n2c4ccccc4c4c2ccc2c5ccccc5n(-c5nc(-c6ccccc6)nc(-c6ccccc6)n5)c24)c2ccccc2n3-c2ccccc2)c([2H])c1[2H] ; [2H]c1c([2H])c([2H])c(-c2nc(-c3ccccc3)nc(-n3c4ccccc4c4ccc5c(c6ccccc6n5-c5cc(-c6ccccc6)cc6c5c5ccccc5n6-c5ccccc5)c43)n2)c([2H])c1[2H] ; [2H]c1c([2H])c([2H])c2c(c1[2H])c1c(c([2H])c([2H])c3c4c([2H])c([2H])c([2H])c([2H])c4n(-c4nc(-c5ccccc5)nc(-c5ccccc5)n4)c31)n2-c1ccc(-c2ccccc2)c2c1c1ccccc1n2-c1ccccc1]

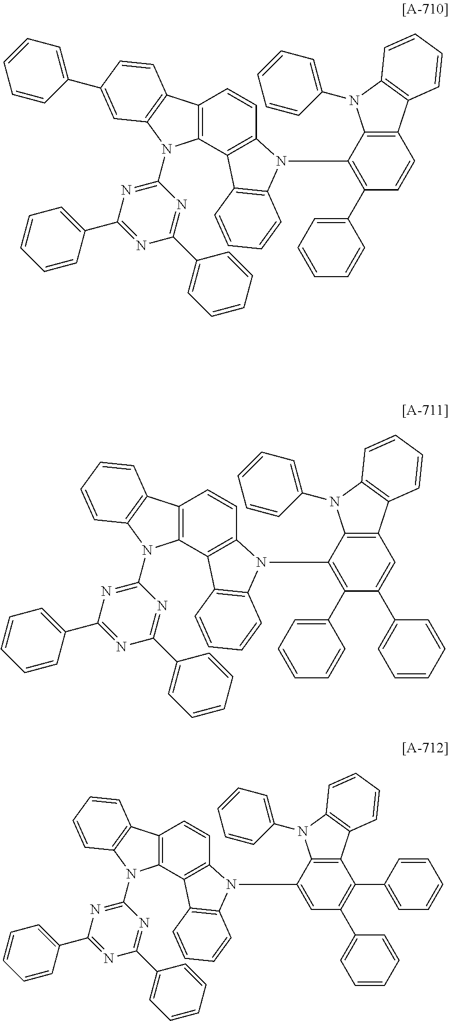 [SMILES: c1ccc(-c2ccc3c4ccc5c(c6ccccc6n5-c5c(-c6ccccc6)ccc6c7ccccc7n(-c7ccccc7)c56)c4n(-c4nc(-c5ccccc5)nc(-c5ccccc5)n4)c3c2)cc1 ; c1ccc(-c2nc(-c3ccccc3)nc(-n3c4ccccc4c4ccc5c(c6ccccc6n5-c5c(-c6ccccc6)c(-c6ccccc6)cc6c7ccccc7n(-c7ccccc7)c56)c43)n2)cc1 ; c1ccc(-c2nc(-c3ccccc3)nc(-n3c4ccccc4c4ccc5c(c6ccccc6n5-c5cc(-c6ccccc6)c(-c6ccccc6)c6c7ccccc7n(-c7ccccc7)c56)c43)n2)cc1]

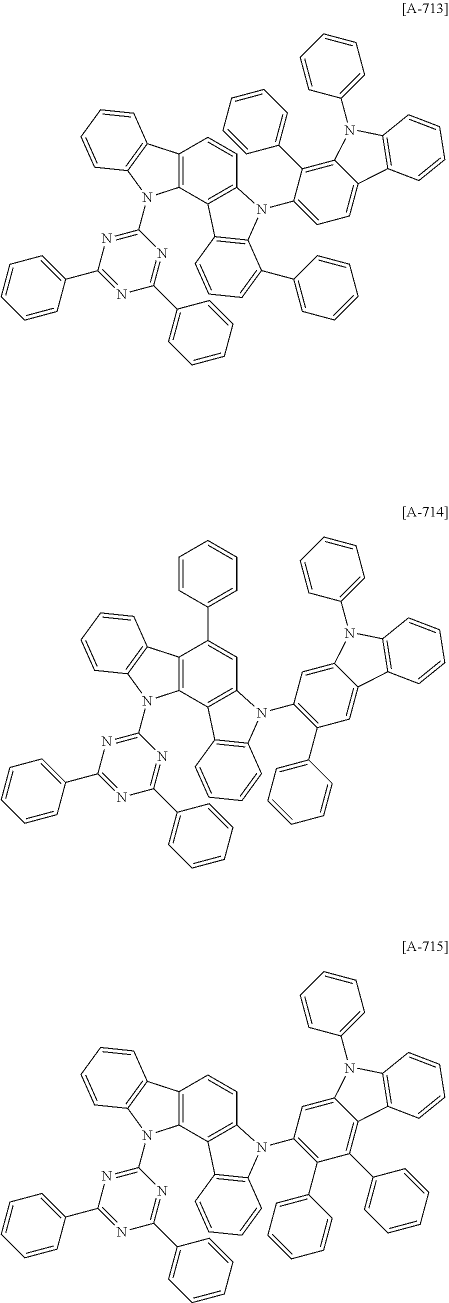 [SMILES: c1ccc(-c2nc(-c3ccccc3)nc(-n3c4ccccc4c4ccc5c(c6cccc(-c7ccccc7)c6n5-c5ccc6c7ccccc7n(-c7ccccc7)c6c5-c5ccccc5)c43)n2)cc1 ; c1ccc(-c2nc(-c3ccccc3)nc(-n3c4ccccc4c4c(-c5ccccc5)cc5c(c6ccccc6n5-c5cc6c(cc5-c5ccccc5)c5ccccc5n6-c5ccccc5)c43)n2)cc1 ; c1ccc(-c2nc(-c3ccccc3)nc(-n3c4ccccc4c4ccc5c(c6ccccc6n5-c5cc6c(c(-c7ccccc7)c5-c5ccccc5)c5ccccc5n6-c5ccccc5)c43)n2)cc1]

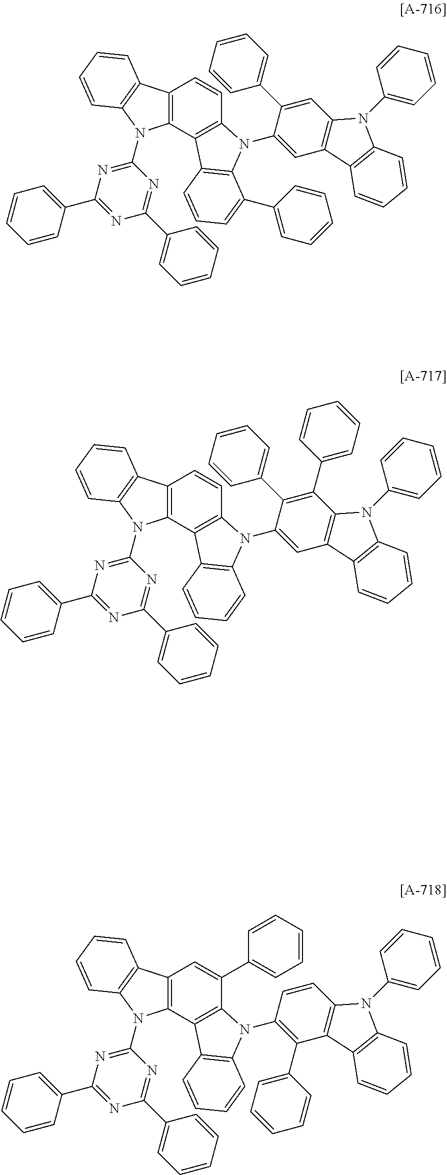 [SMILES: c1ccc(-c2nc(-c3ccccc3)nc(-n3c4ccccc4c4ccc5c(c6cccc(-c7ccccc7)c6n5-c5cc6c7ccccc7n(-c7ccccc7)c6cc5-c5ccccc5)c43)n2)cc1 ; c1ccc(-c2nc(-c3ccccc3)nc(-n3c4ccccc4c4ccc5c(c6ccccc6n5-c5cc6c7ccccc7n(-c7ccccc7)c6c(-c6ccccc6)c5-c5ccccc5)c43)n2)cc1 ; c1ccc(-c2nc(-c3ccccc3)nc(-n3c4ccccc4c4cc(-c5ccccc5)c5c(c6ccccc6n5-c5ccc6c(c5-c5ccccc5)c5ccccc5n6-c5ccccc5)c43)n2)cc1]

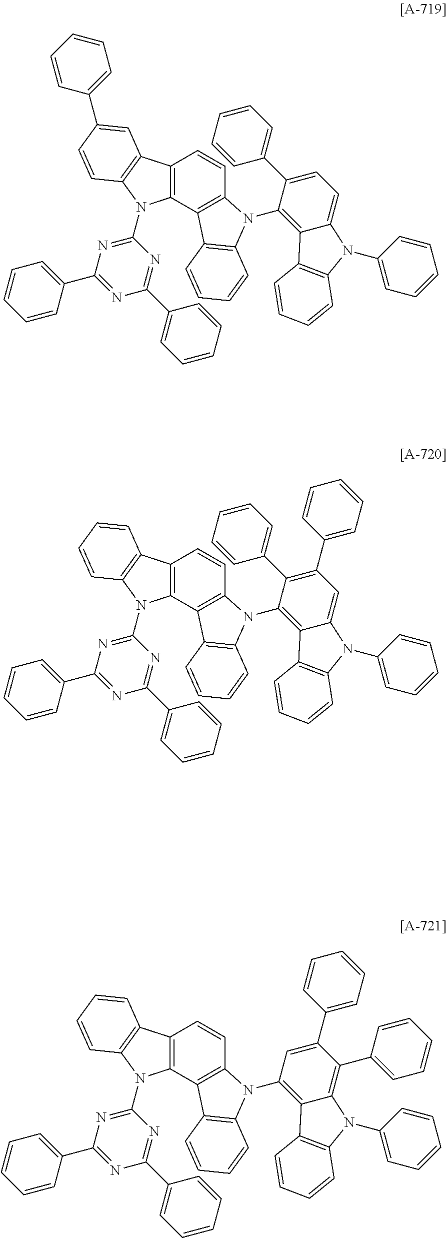 [SMILES: c1ccc(-c2ccc3c(c2)c2ccc4c(c5ccccc5n4-c4c(-c5ccccc5)ccc5c4c4ccccc4n5-c4ccccc4)c2n3-c2nc(-c3ccccc3)nc(-c3ccccc3)n2)cc1 ; c1ccc(-c2nc(-c3ccccc3)nc(-n3c4ccccc4c4ccc5c(c6ccccc6n5-c5c(-c6ccccc6)c(-c6ccccc6)cc6c5c5ccccc5n6-c5ccccc5)c43)n2)cc1 ; c1ccc(-c2nc(-c3ccccc3)nc(-n3c4ccccc4c4ccc5c(c6ccccc6n5-c5cc(-c6ccccc6)c(-c6ccccc6)c6c5c5ccccc5n6-c5ccccc5)c43)n2)cc1]

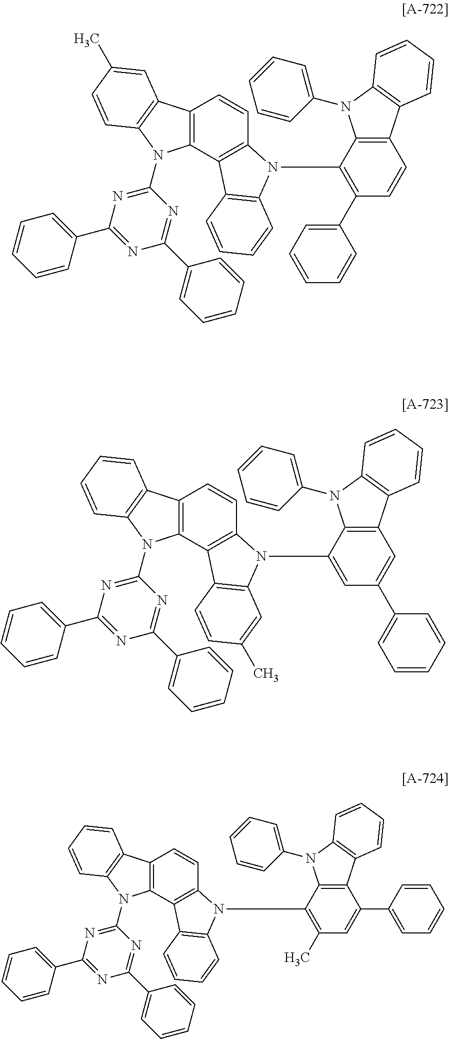 [SMILES: Cc1ccc2c(c1)c1ccc3c(c4ccccc4n3-c3c(-c4ccccc4)ccc4c5ccccc5n(-c5ccccc5)c34)c1n2-c1nc(-c2ccccc2)nc(-c2ccccc2)n1 ; Cc1ccc2c3c(ccc4c5ccccc5n(-c5nc(-c6ccccc6)nc(-c6ccccc6)n5)c43)n(-c3cc(-c4ccccc4)cc4c5ccccc5n(-c5ccccc5)c34)c2c1 ; Cc1cc(-c2ccccc2)c2c3ccccc3n(-c3ccccc3)c2c1-n1c2ccccc2c2c1ccc1c3ccccc3n(-c3nc(-c4ccccc4)nc(-c4ccccc4)n3)c12]

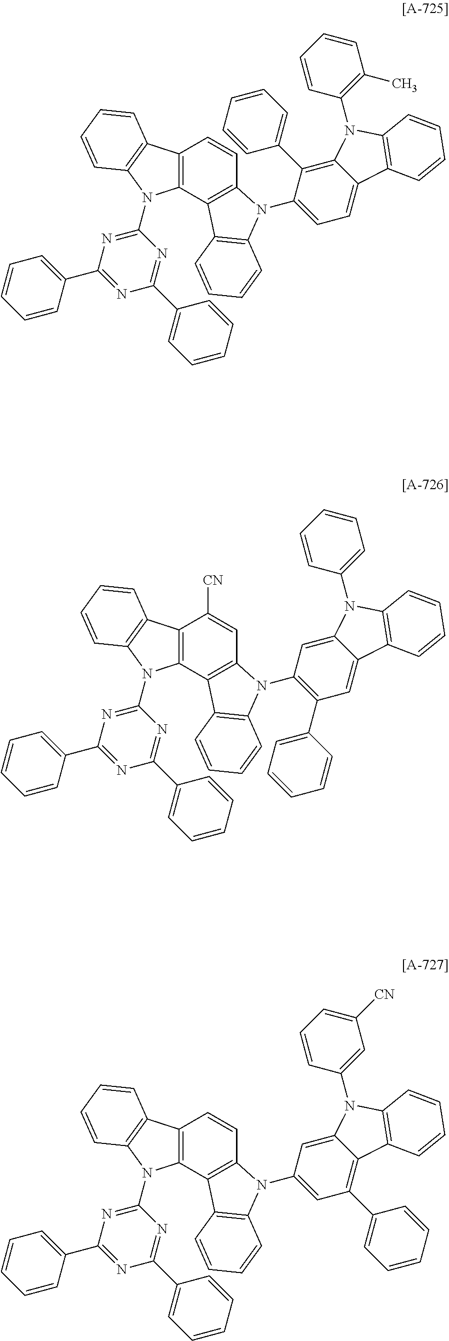 [SMILES: Cc1ccccc1-n1c2ccccc2c2ccc(-n3c4ccccc4c4c3ccc3c5ccccc5n(-c5nc(-c6ccccc6)nc(-c6ccccc6)n5)c34)c(-c3ccccc3)c21 ; N#Cc1cc2c(c3ccccc3n2-c2cc3c(cc2-c2ccccc2)c2ccccc2n3-c2ccccc2)c2c1c1ccccc1n2-c1nc(-c2ccccc2)nc(-c2ccccc2)n1 ; N#Cc1cccc(-n2c3ccccc3c3c(-c4ccccc4)cc(-n4c5ccccc5c5c4ccc4c6ccccc6n(-c6nc(-c7ccccc7)nc(-c7ccccc7)n6)c45)cc32)c1]

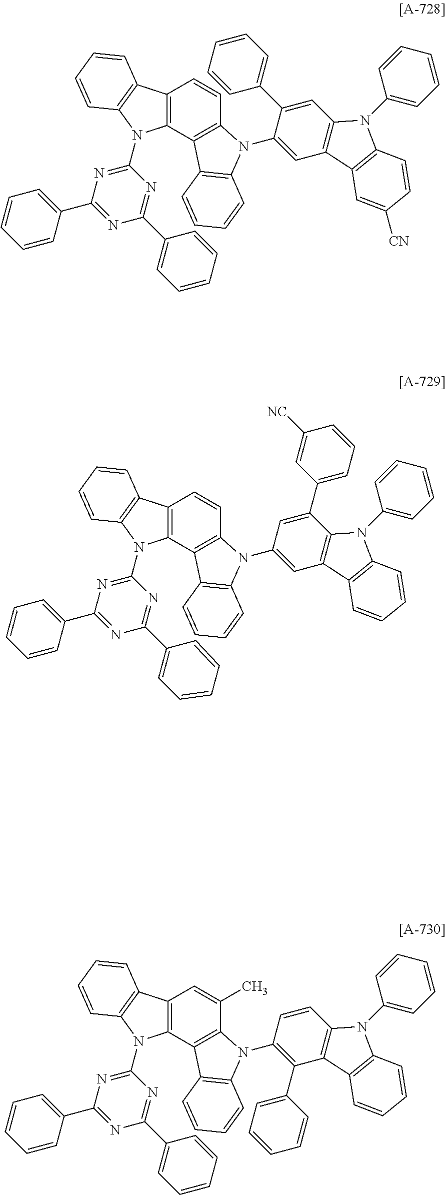 [SMILES: N#Cc1ccc2c(c1)c1cc(-n3c4ccccc4c4c3ccc3c5ccccc5n(-c5nc(-c6ccccc6)nc(-c6ccccc6)n5)c34)c(-c3ccccc3)cc1n2-c1ccccc1 ; N#Cc1cccc(-c2cc(-n3c4ccccc4c4c3ccc3c5ccccc5n(-c5nc(-c6ccccc6)nc(-c6ccccc6)n5)c34)cc3c4ccccc4n(-c4ccccc4)c23)c1 ; Cc1cc2c3ccccc3n(-c3nc(-c4ccccc4)nc(-c4ccccc4)n3)c2c2c3ccccc3n(-c3ccc4c(c3-c3ccccc3)c3ccccc3n4-c3ccccc3)c12]

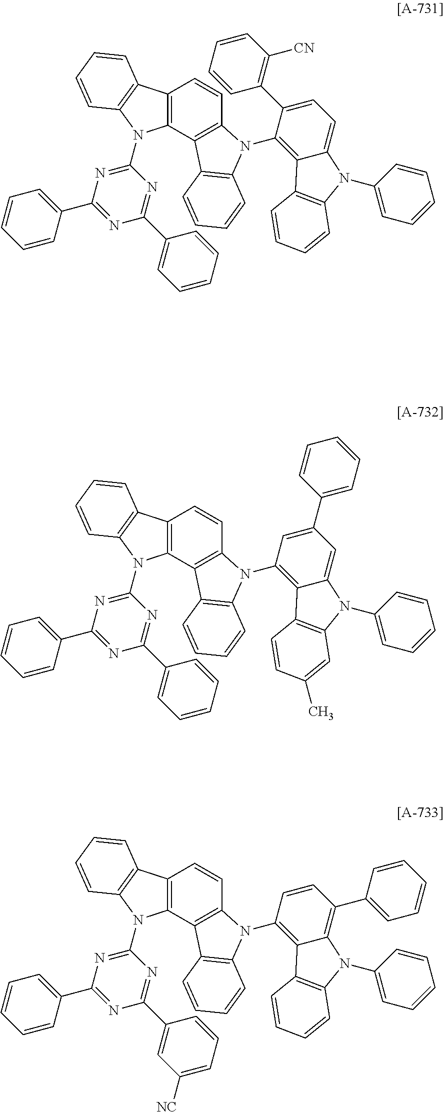 [SMILES: N#Cc1ccccc1-c1ccc2c(c1-n1c3ccccc3c3c1ccc1c4ccccc4n(-c4nc(-c5ccccc5)nc(-c5ccccc5)n4)c13)c1ccccc1n2-c1ccccc1 ; Cc1ccc2c3c(-n4c5ccccc5c5c4ccc4c6ccccc6n(-c6nc(-c7ccccc7)nc(-c7ccccc7)n6)c45)cc(-c4ccccc4)cc3n(-c3ccccc3)c2c1 ; N#Cc1cccc(-c2nc(-c3ccccc3)nc(-n3c4ccccc4c4ccc5c(c6ccccc6n5-c5ccc(-c6ccccc6)c6c5c5ccccc5n6-c5ccccc5)c43)n2)c1]

A composition for an organic optoelectronic device according to another embodiment includes a first compound, and a second compound, wherein the first compound is the aforementioned compound for an organic optoelectronic device, and the second compound is a compound for an organic optoelectronic device represented by Chemical Formula 3; or a compound for an organic optoelectronic device represented by a combination of Chemical Formula 4 and Chemical Formula 5.

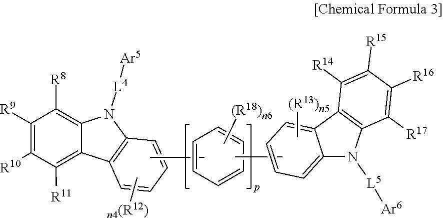

In Chemical Formula 3,
Ar⁵ and Ar⁶ are each independently a substituted or unsubstituted C6 to C20 aryl group or a substituted or unsubstituted C2 to C30 heterocyclic group,
L⁴ and L⁵ are each independently a single bond or a substituted or unsubstituted C6 to C20 arylene group,
R⁸ to R¹⁸ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
n4 and n5 are each independently one of integers of 1 to 3,
n6 is one of integers of 1 to 4, and
p is one of integers of 0 to 2;

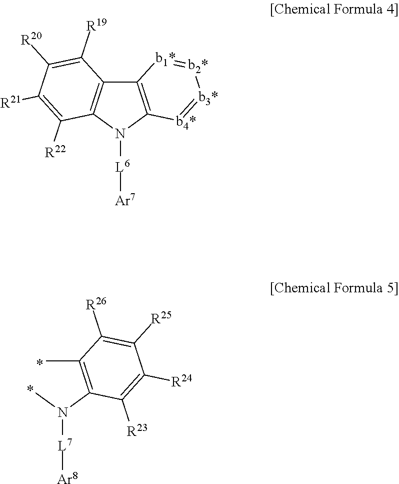

wherein, in Chemical Formulas 4 and 5,
Ar⁷ and Ar⁸ are each independently a substituted or unsubstituted C6 to C20 aryl group or a substituted or unsubstituted C2 to C30 heterocyclic group,
b₁* to b₄* in Chemical Formula 4 are each independently a linking carbon (C) or C-L^{b}-R^{b},
among b₁* to b₄* in Chemical Formula 4, the two adjacent ones are linked to * in Chemical Formula 5,
L^{b}, L⁶, and L⁷ are each independently a single bond or a substituted or unsubstituted C6 to C20 arylene group, and
R^{b} and R¹⁹ to R²⁶ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

The second compound may be used in the light emitting layer together with the first compound to improve luminous efficiency and life-span characteristics by increasing charge mobility and stability.

For example, in Chemical Formula 3, Ar⁵ and Ar⁶ may each independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group,
in Chemical Formula 3, L⁴ and L⁵ may each independently be a single bond, a substituted or unsubstituted phenylene group, or a substituted or unsubstituted biphenylene group,
in Chemical Formula 3, R⁸ to R¹⁸ may each independently be hydrogen, deuterium, or a substituted or unsubstituted C6 to C12 aryl group, and
p may be 0 or 1.

As an example, “substituted” in Chemical Formula 3 refers to replacement of at least one hydrogen by deuterium, a C1 to C4 alkyl group, a C6 to C18 aryl group, or a C2 to C30 heteroaryl group.

In a specific embodiment of the present invention, Chemical Formula 3 may be represented by one of Chemical Formula 3-1 to Chemical Formula 3-15.

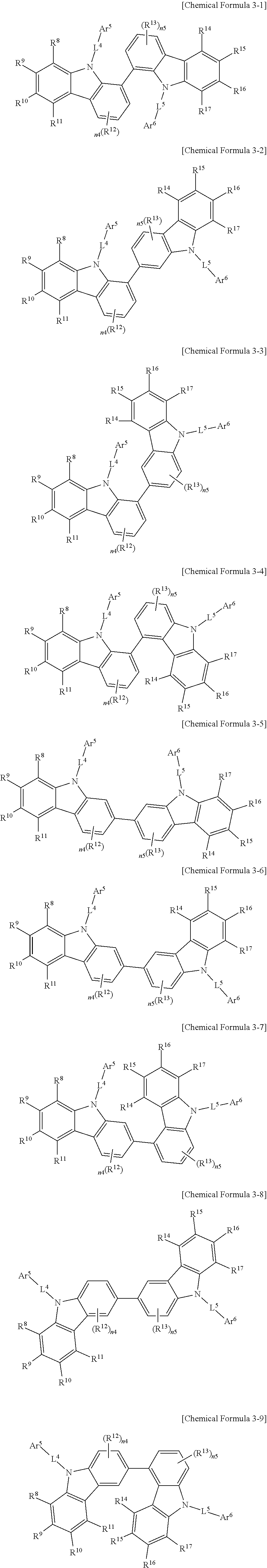

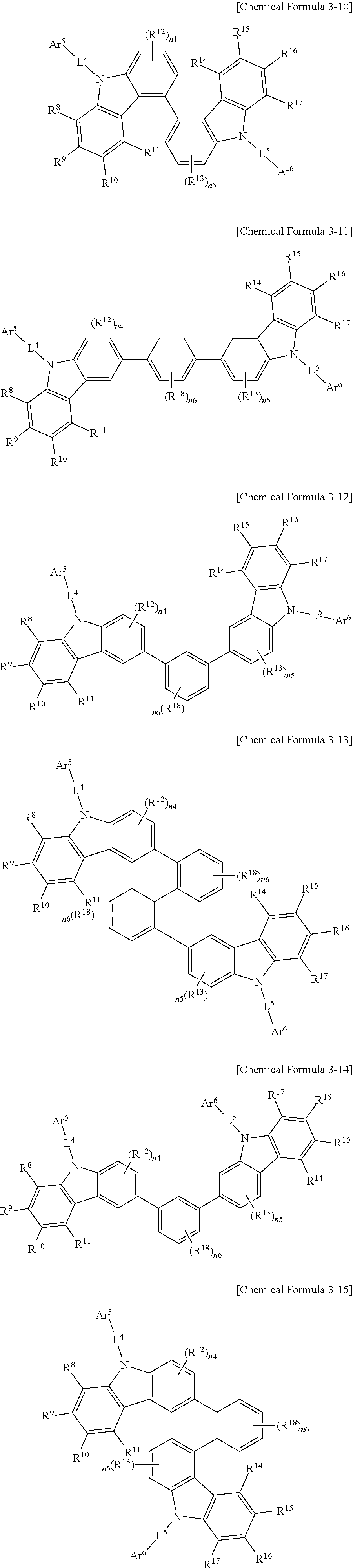

In Chemical Formula 3-1 to Chemical Formula 3-15, n4 to n6 are the same as described above, R⁸ to R¹⁸ may each independently be hydrogen, deuterium, or a substituted or unsubstituted C6 to C12 aryl group, and *-L⁴-Ar⁵ and *-L⁵-Ar⁶ may each independently be one of substituents listed in Group II.

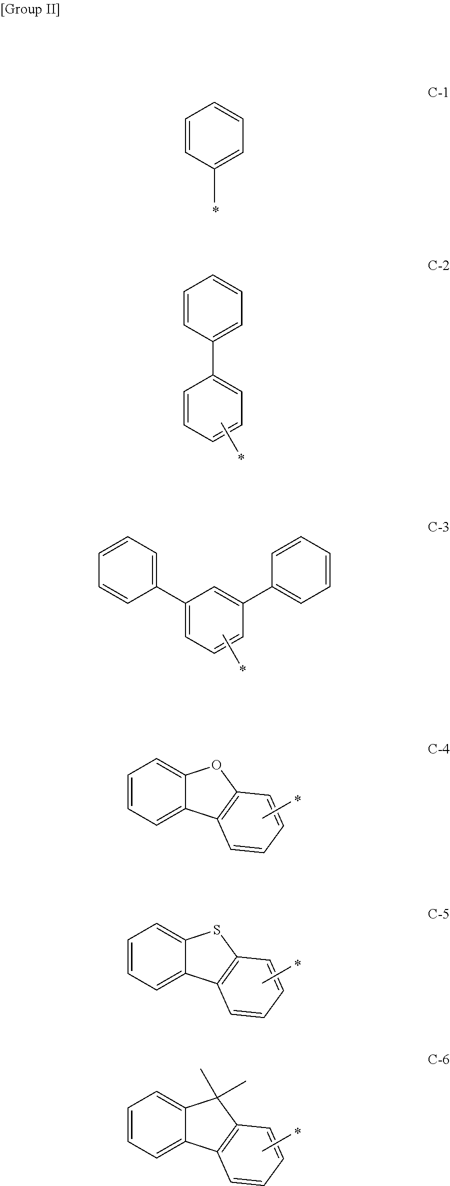

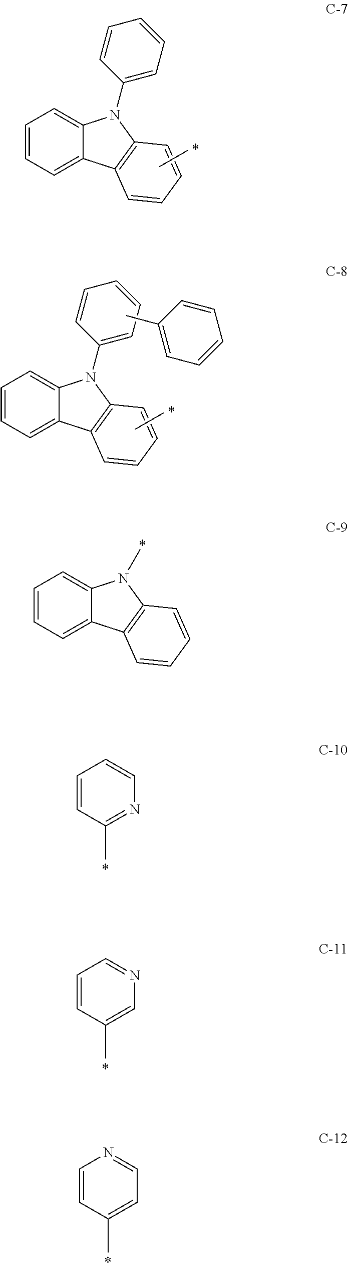

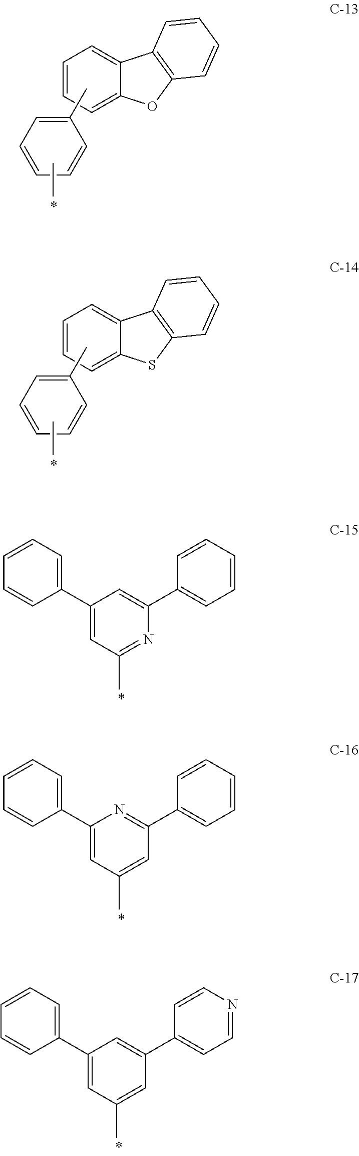

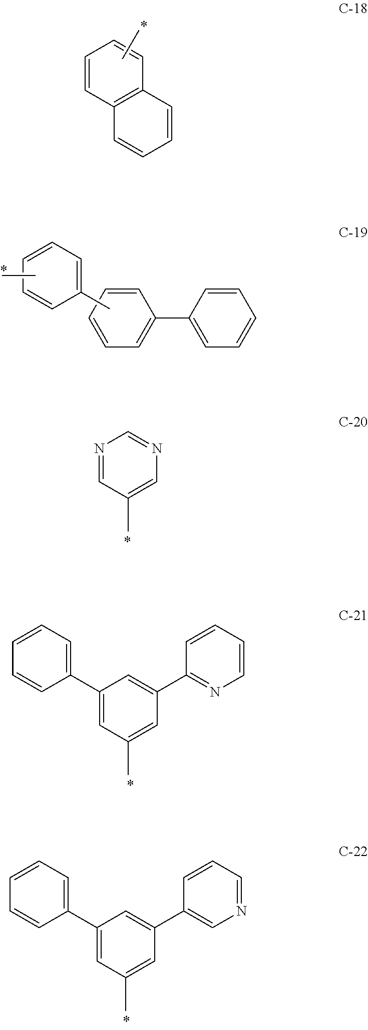

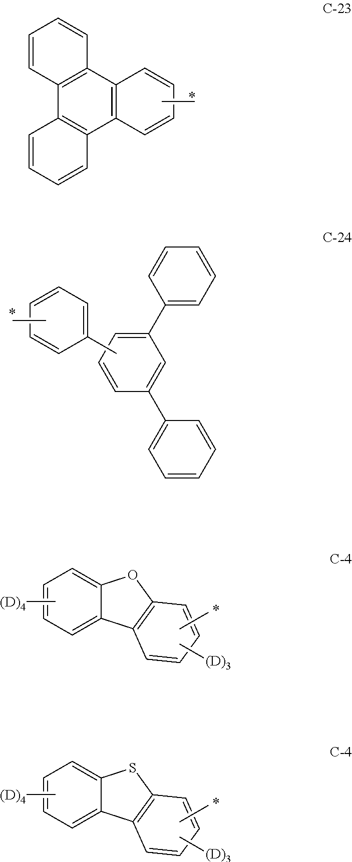

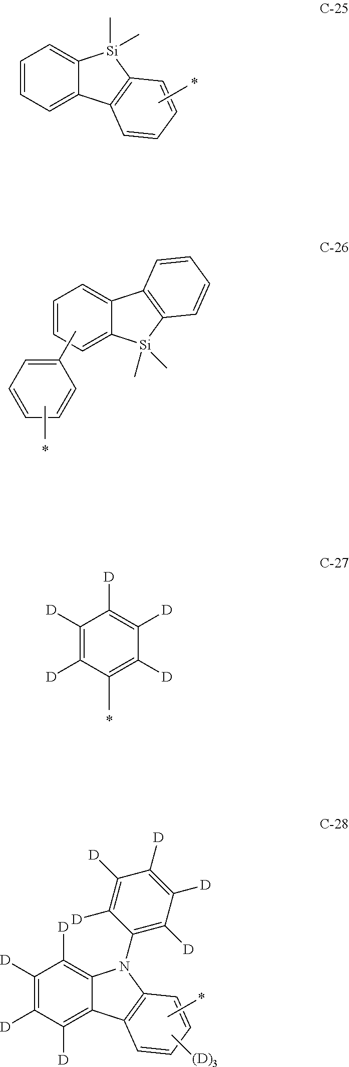

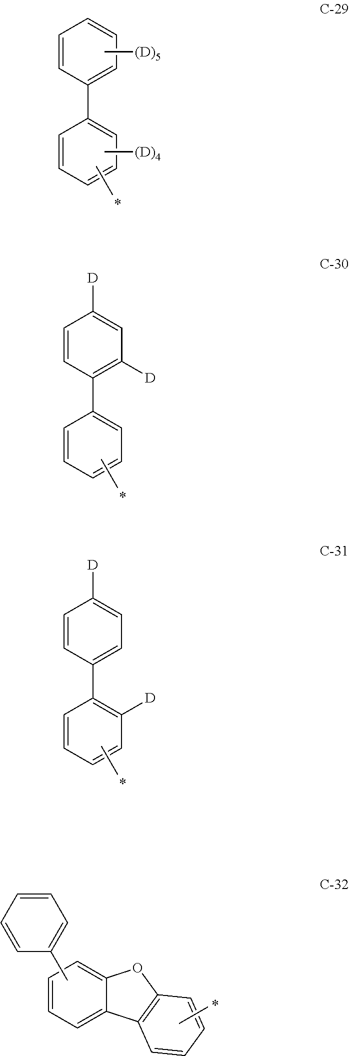

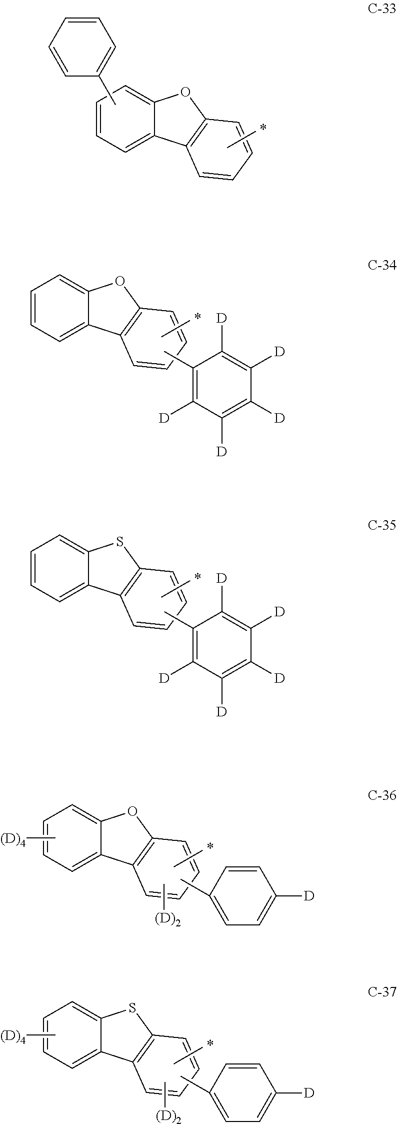

In Group II, * is a linking point.

In an embodiment, Chemical Formula 3 may be represented by Chemical Formula 3-8.

In addition, *-L⁴-Ar⁵ and *-L⁵-Ar⁶ of Chemical Formula 3-8 may each independently be selected from Group II, for example C-1, C-2, C-3, C-4, C-7, C-8, and C-9.

As an example, the second compound represented by the combination of Chemical Formula 4 and Chemical Formula 5 may be represented by any one of Chemical Formula Chemical Formula 4A, Chemical Formula 4B, Chemical Formula 4C, Chemical Formula 4D, and Chemical Formula 4E.

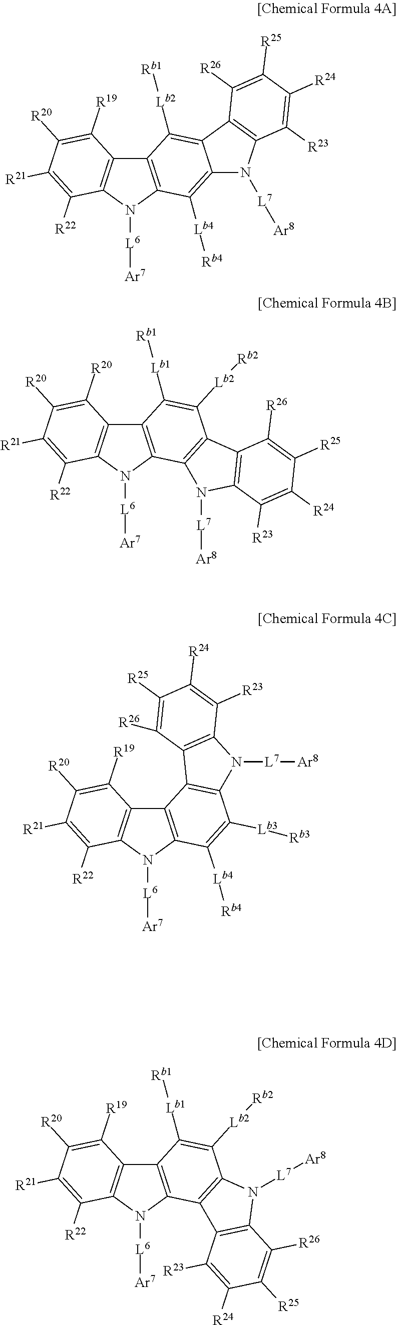

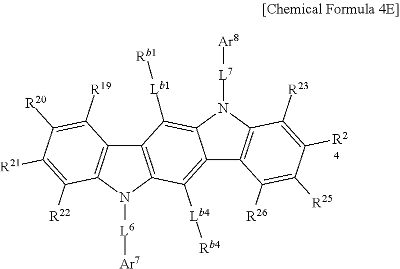

In Chemical Formula 4A to Chemical Formula 4E, Ar⁷, Ar⁸, L⁶, L⁷, and R¹⁹ to R²⁶ are the same as described above,
the definitions of L^{b1} to L^{b4} are the same as the definitions of L⁶ and L⁷, and
R^{b1} to R^{b4} are the same as the definitions of R¹⁹ to R²⁶.

For example, in Chemical Formula 4 and 5, Ar⁷ and Ar⁸ may each independently be a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group, and
R^{b1} to R^{b4} and R¹⁹ to R²⁶ may each independently be hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In a specific embodiment of the present invention, Ar⁷ and Ar⁸ in Chemical Formulas 4 and 5 may each be independently selected from the substituents listed in Group II.

In an embodiment, R^{b1} to R^{b4} and R¹⁹ to R²⁶ may each independently be hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

For example, R^{b1} to R^{b4} and R¹⁹ to R²⁶ may each independently be hydrogen, deuterium, a cyano group, or a substituted or unsubstituted phenyl group, and
in a specific embodiment, R^{b1} to R^{b4}, and R¹⁹ to R²⁶ may each independently be hydrogen or a phenyl group.

In a specific embodiment of the present invention, the second compound may be represented by Chemical Formula 3-8, and in Chemical Formula 3-8, n4 and n5 may be the same as described above, Ar⁵ and Ar⁶ may each independently be a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group, L⁴ and L⁵ may each independently be a single bond, or a substituted or unsubstituted C6 to C20 arylene group, and R⁸ to R¹⁷ may each independently be hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In another specific embodiment of the present invention, the second compound may be represented by Chemical Formula 4C, wherein in Chemical Formula 4C, L^{b3} and L^{b4} may be a single bond, L⁶ and L⁷ may each independently be a single bond or a substituted or unsubstituted C6 to C12 arylene group, R¹⁹ to R²⁶, R^{b3}, and R^{b4} may each be hydrogen or a phenyl group, and Ar⁷ and Ar⁸ may each independently be a substituted or unsubstituted phenyl group, a substituted or unsubstituted biphenyl group, or a substituted or unsubstituted terphenyl group.

For example, the second compound may be one selected from the compounds listed in Group 2, but is not limited thereto.

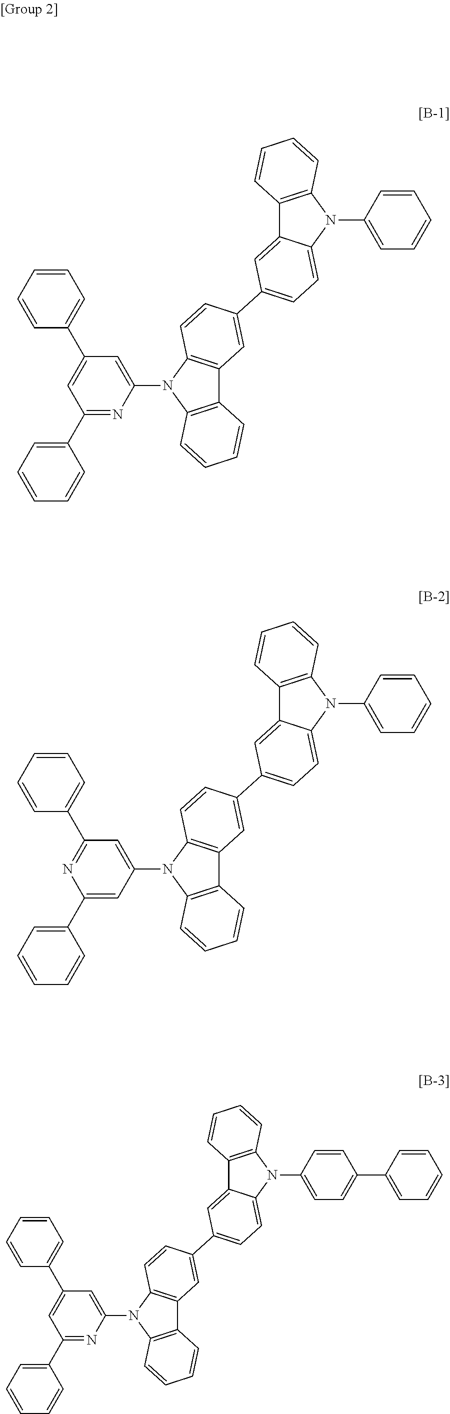 [SMILES: c1ccc(-c2cc(-c3ccccc3)nc(-n3c4ccccc4c4cc(-c5ccc6c(c5)c5ccccc5n6-c5ccccc5)ccc43)c2)cc1 ; c1ccc(-c2cc(-n3c4ccccc4c4cc(-c5ccc6c(c5)c5ccccc5n6-c5ccccc5)ccc43)cc(-c3ccccc3)n2)cc1 ; c1ccc(-c2ccc(-n3c4ccccc4c4cc(-c5ccc6c(c5)c5ccccc5n6-c5cc(-c6ccccc6)cc(-c6ccccc6)n5)ccc43)cc2)cc1]

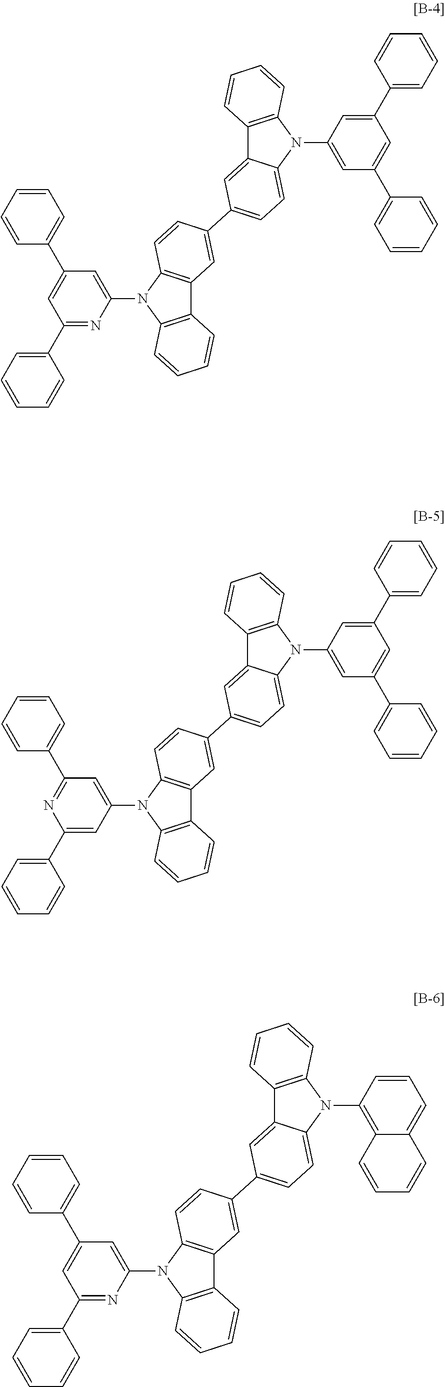 [SMILES: c1ccc(-c2cc(-c3ccccc3)cc(-n3c4ccccc4c4cc(-c5ccc6c(c5)c5ccccc5n6-c5cc(-c6ccccc6)cc(-c6ccccc6)n5)ccc43)c2)cc1 ; c1ccc(-c2cc(-c3ccccc3)cc(-n3c4ccccc4c4cc(-c5ccc6c(c5)c5ccccc5n6-c5cc(-c6ccccc6)nc(-c6ccccc6)c5)ccc43)c2)cc1 ; c1ccc(-c2cc(-c3ccccc3)nc(-n3c4ccccc4c4cc(-c5ccc6c(c5)c5ccccc5n6-c5cccc6ccccc56)ccc43)c2)cc1]

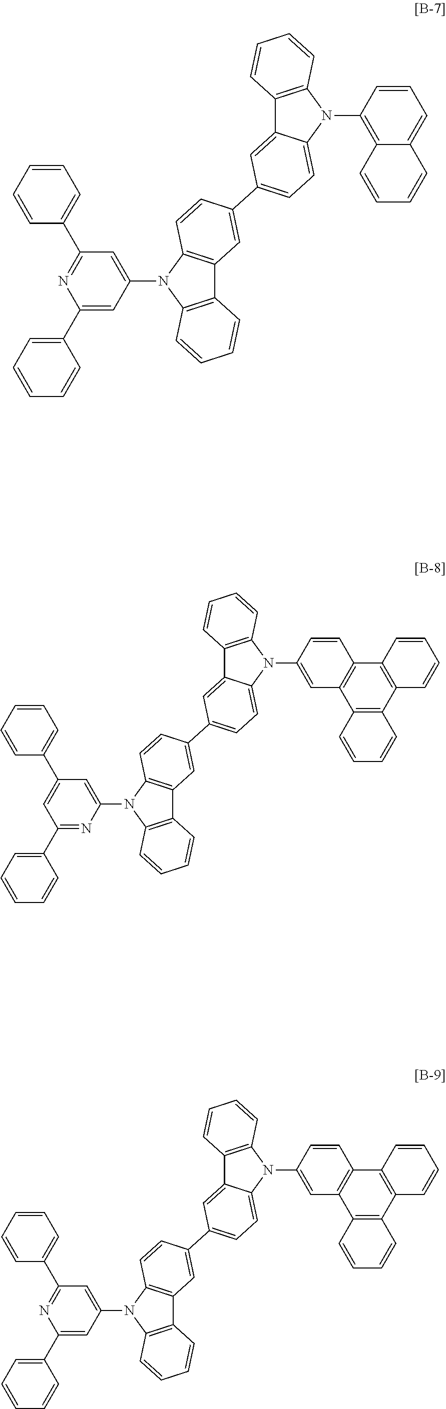 [SMILES: c1ccc(-c2cc(-n3c4ccccc4c4cc(-c5ccc6c(c5)c5ccccc5n6-c5cccc6ccccc56)ccc43)cc(-c3ccccc3)n2)cc1 ; c1ccc(-c2cc(-c3ccccc3)nc(-n3c4ccccc4c4cc(-c5ccc6c(c5)c5ccccc5n6-c5ccc6c7ccccc7c7ccccc7c6c5)ccc43)c2)cc1 ; c1ccc(-c2cc(-n3c4ccccc4c4cc(-c5ccc6c(c5)c5ccccc5n6-c5ccc6c7ccccc7c7ccccc7c6c5)ccc43)cc(-c3ccccc3)n2)cc1]

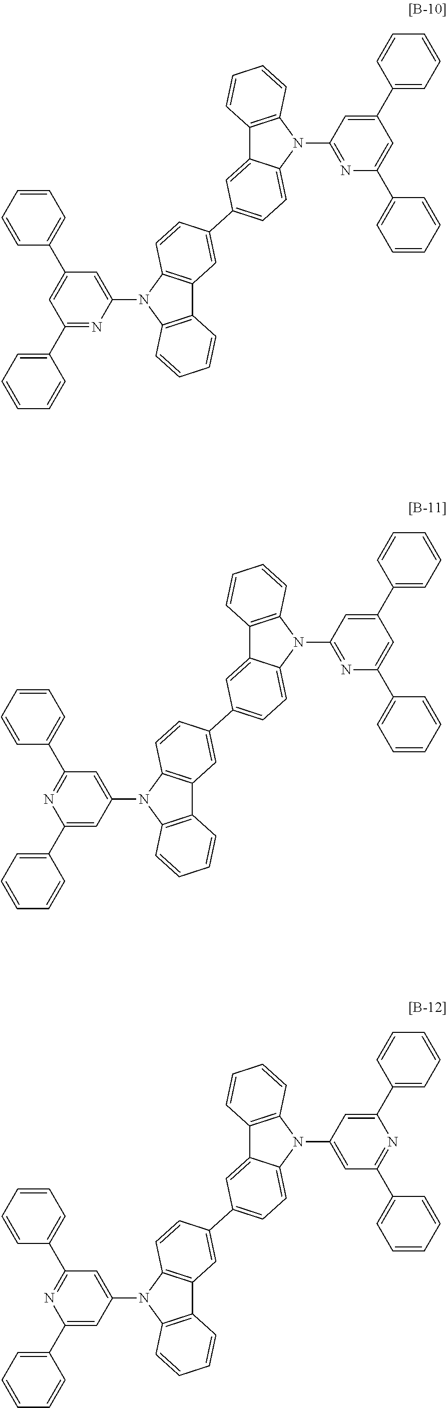 [SMILES: c1ccc(-c2cc(-c3ccccc3)nc(-n3c4ccccc4c4cc(-c5ccc6c(c5)c5ccccc5n6-c5cc(-c6ccccc6)cc(-c6ccccc6)n5)ccc43)c2)cc1 ; c1ccc(-c2cc(-c3ccccc3)nc(-n3c4ccccc4c4cc(-c5ccc6c(c5)c5ccccc5n6-c5cc(-c6ccccc6)nc(-c6ccccc6)c5)ccc43)c2)cc1 ; c1ccc(-c2cc(-n3c4ccccc4c4cc(-c5ccc6c(c5)c5ccccc5n6-c5cc(-c6ccccc6)nc(-c6ccccc6)c5)ccc43)cc(-c3ccccc3)n2)cc1]

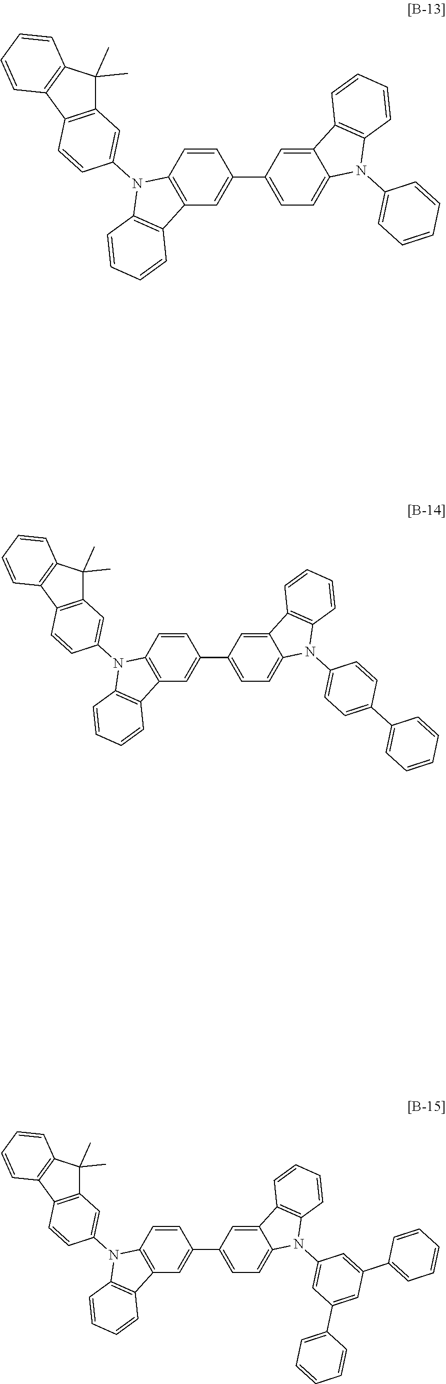 [SMILES: CC1(C)c2ccccc2-c2ccc(-n3c4ccccc4c4cc(-c5ccc6c(c5)c5ccccc5n6-c5ccccc5)ccc43)cc21 ; CC1(C)c2ccccc2-c2ccc(-n3c4ccccc4c4cc(-c5ccc6c(c5)c5ccccc5n6-c5ccc(-c6ccccc6)cc5)ccc43)cc21 ; CC1(C)c2ccccc2-c2ccc(-n3c4ccccc4c4cc(-c5ccc6c(c5)c5ccccc5n6-c5cc(-c6ccccc6)cc(-c6ccccc6)c5)ccc43)cc21]

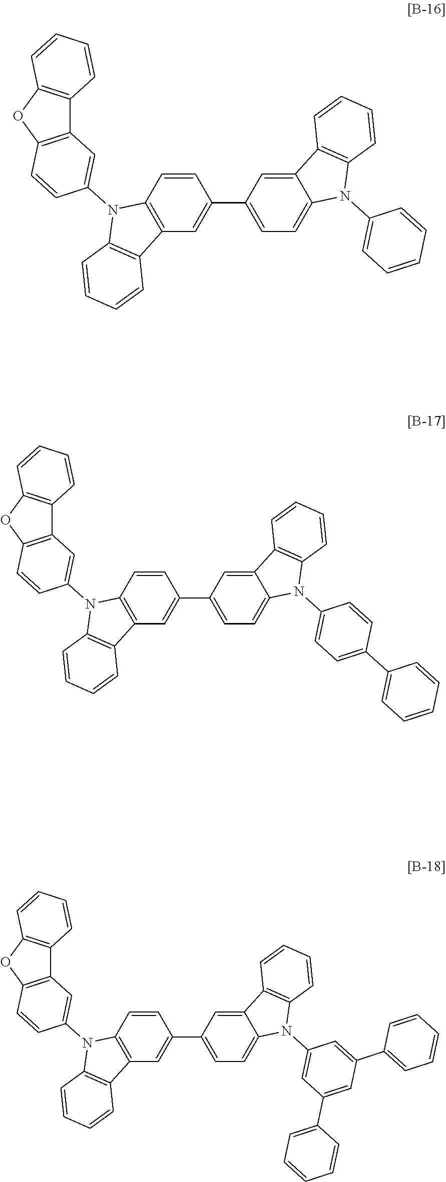 [SMILES: c1ccc(-n2c3ccccc3c3cc(-c4ccc5c(c4)c4ccccc4n5-c4ccc5oc6ccccc6c5c4)ccc32)cc1 ; c1ccc(-c2ccc(-n3c4ccccc4c4cc(-c5ccc6c(c5)c5ccccc5n6-c5ccc6oc7ccccc7c6c5)ccc43)cc2)cc1 ; c1ccc(-c2cc(-c3ccccc3)cc(-n3c4ccccc4c4cc(-c5ccc6c(c5)c5ccccc5n6-c5ccc6oc7ccccc7c6c5)ccc43)c2)cc1]

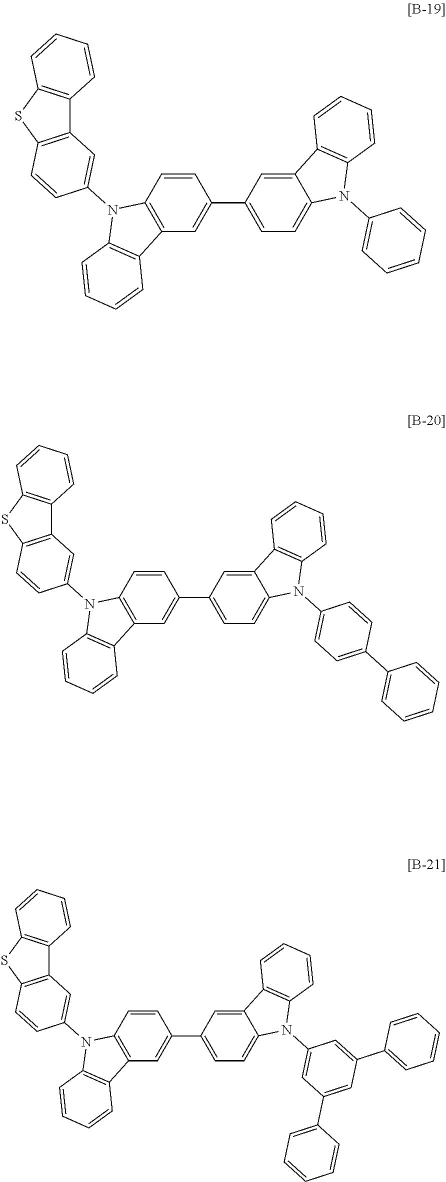 [SMILES: c1ccc(-n2c3ccccc3c3cc(-c4ccc5c(c4)c4ccccc4n5-c4ccc5sc6ccccc6c5c4)ccc32)cc1 ; c1ccc(-c2ccc(-n3c4ccccc4c4cc(-c5ccc6c(c5)c5ccccc5n6-c5ccc6sc7ccccc7c6c5)ccc43)cc2)cc1 ; c1ccc(-c2cc(-c3ccccc3)cc(-n3c4ccccc4c4cc(-c5ccc6c(c5)c5ccccc5n6-c5ccc6sc7ccccc7c6c5)ccc43)c2)cc1]

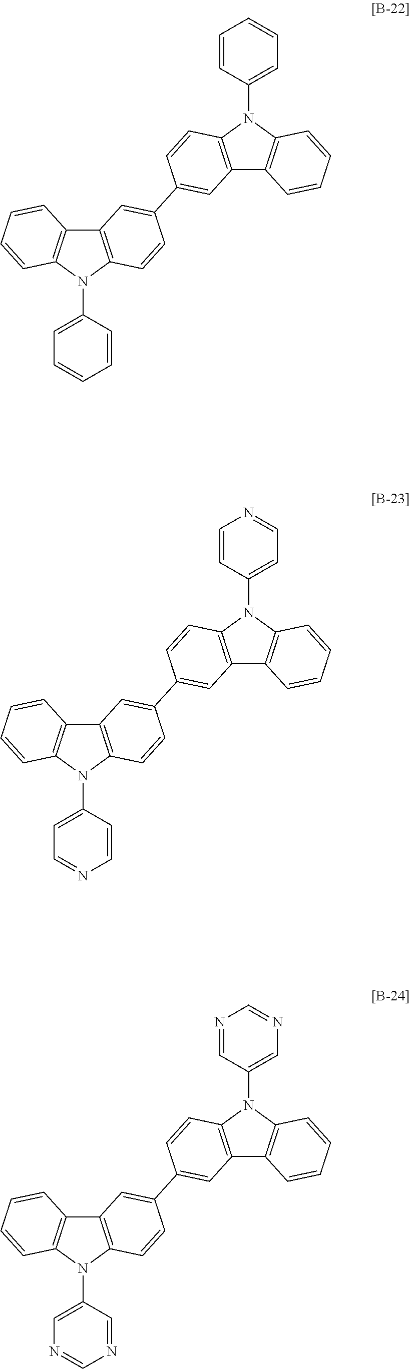 [SMILES: c1ccc(-n2c3ccccc3c3cc(-c4ccc5c(c4)c4ccccc4n5-c4ccccc4)ccc32)cc1 ; c1ccc2c(c1)c1cc(-c3ccc4c(c3)c3ccccc3n4-c3ccncc3)ccc1n2-c1ccncc1 ; c1ccc2c(c1)c1cc(-c3ccc4c(c3)c3ccccc3n4-c3cncnc3)ccc1n2-c1cncnc1]

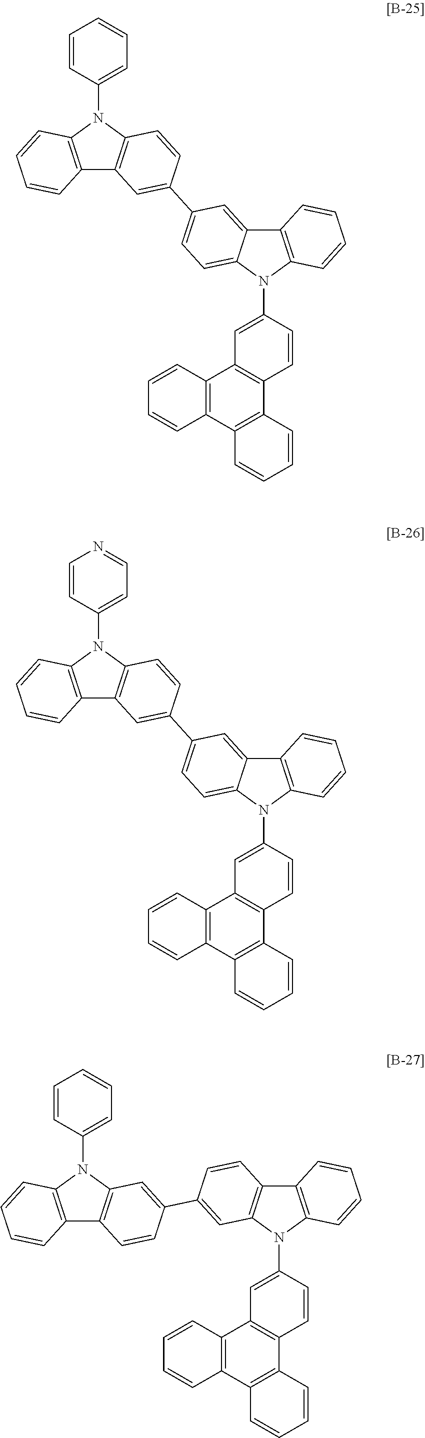 [SMILES: c1ccc(-n2c3ccccc3c3cc(-c4ccc5c(c4)c4ccccc4n5-c4ccc5c6ccccc6c6ccccc6c5c4)ccc32)cc1 ; c1ccc2c(c1)c1ccccc1c1cc(-n3c4ccccc4c4cc(-c5ccc6c(c5)c5ccccc5n6-c5ccncc5)ccc43)ccc21 ; c1ccc(-n2c3ccccc3c3ccc(-c4ccc5c6ccccc6n(-c6ccc7c8ccccc8c8ccccc8c7c6)c5c4)cc32)cc1]

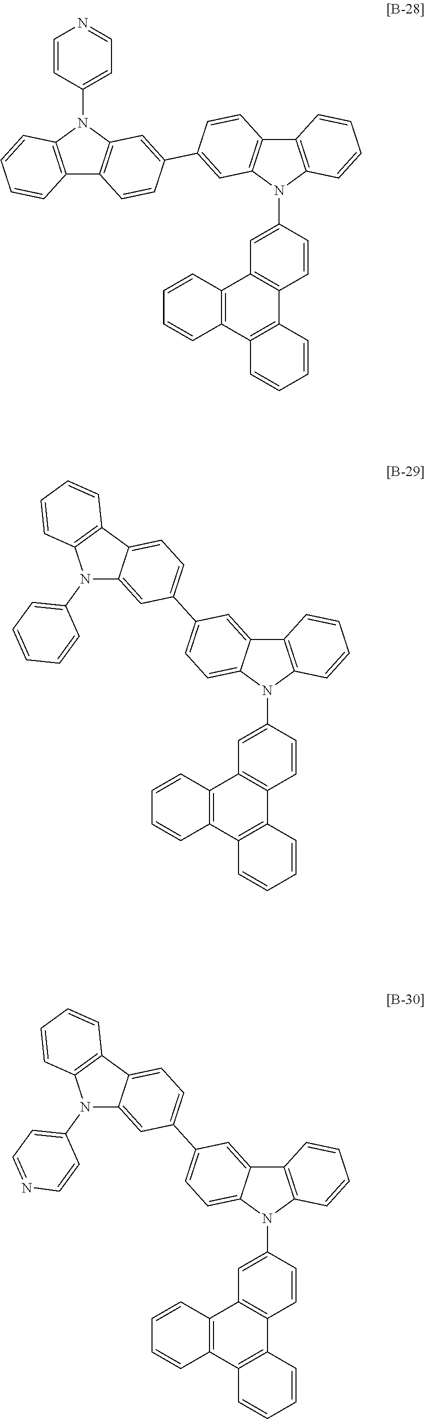 [SMILES: c1ccc2c(c1)c1ccccc1c1cc(-n3c4ccccc4c4ccc(-c5ccc6c7ccccc7n(-c7ccncc7)c6c5)cc43)ccc21 ; c1ccc(-n2c3ccccc3c3ccc(-c4ccc5c(c4)c4ccccc4n5-c4ccc5c6ccccc6c6ccccc6c5c4)cc32)cc1 ; c1ccc2c(c1)c1ccccc1c1cc(-n3c4ccccc4c4cc(-c5ccc6c7ccccc7n(-c7ccncc7)c6c5)ccc43)ccc21]

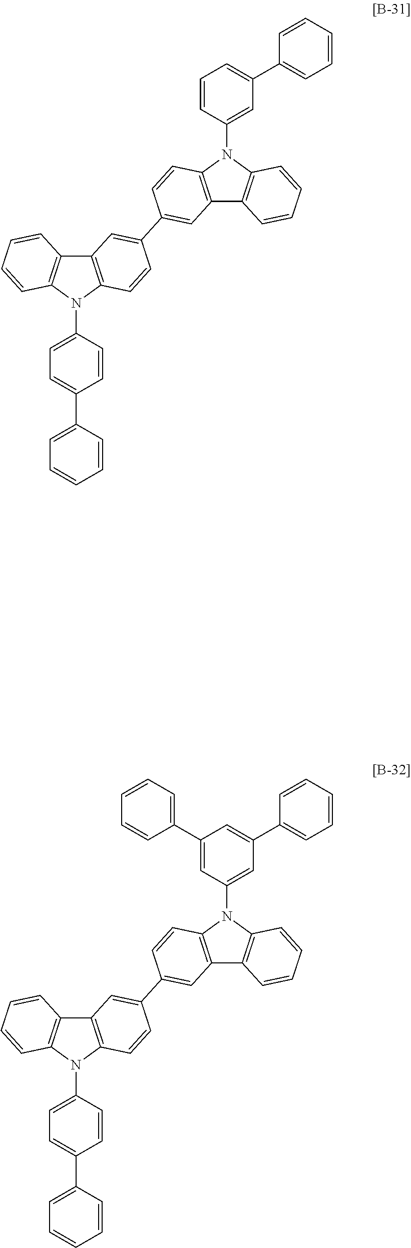 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1]

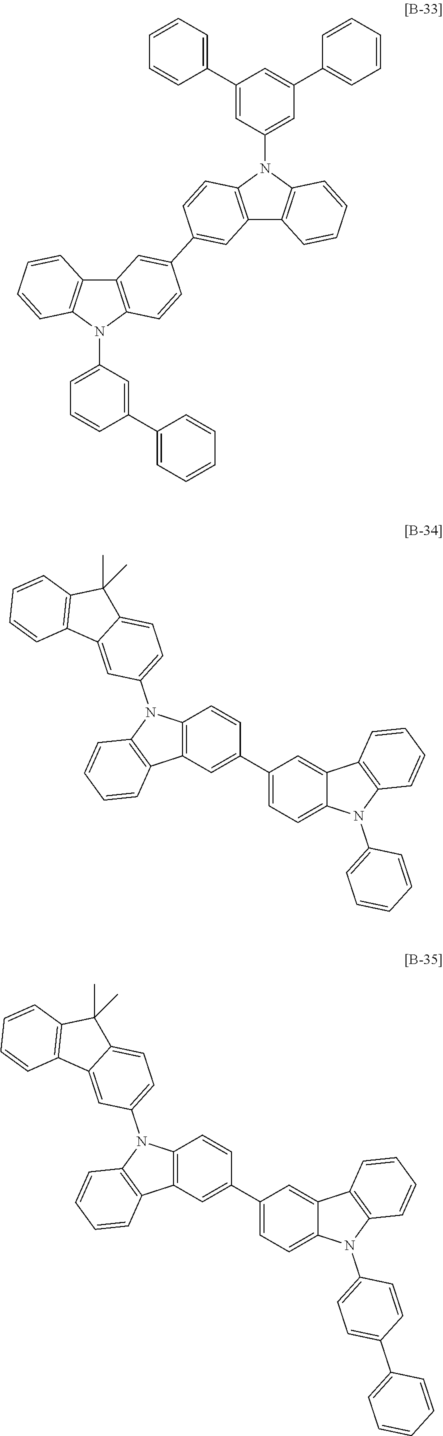 [SMILES: c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; CC1(C)c2ccccc2-c2cc(-n3c4ccccc4c4cc(-c5ccc6c(c5)c5ccccc5n6-c5ccccc5)ccc43)ccc21 ; CC1(C)c2ccccc2-c2cc(-n3c4ccccc4c4cc(-c5ccc6c(c5)c5ccccc5n6-c5ccc(-c6ccccc6)cc5)ccc43)ccc21]

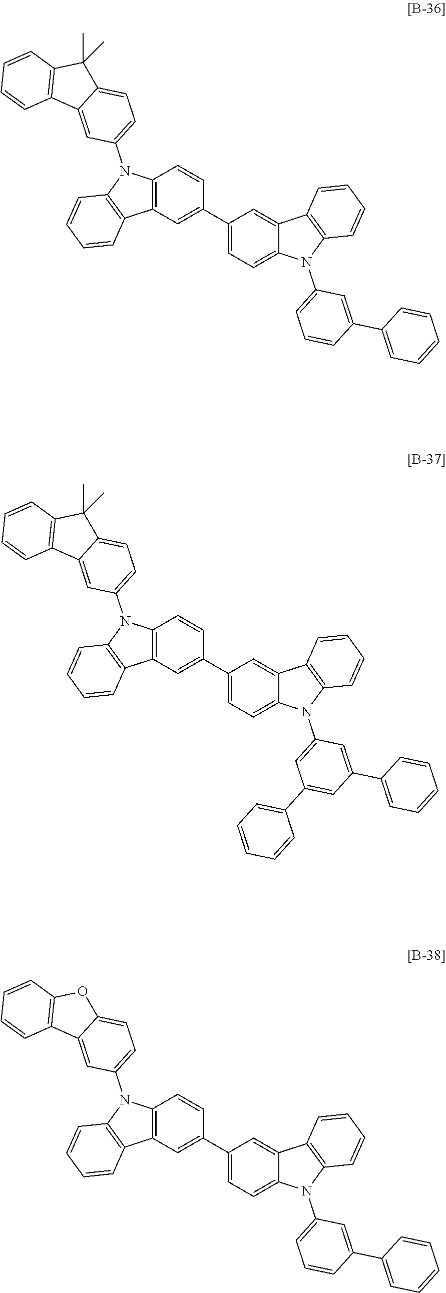 [SMILES: CC1(C)c2ccccc2-c2cc(-n3c4ccccc4c4cc(-c5ccc6c(c5)c5ccccc5n6-c5cccc(-c6ccccc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6ccccc6)c5)ccc43)ccc21 ; c1ccc(-c2cccc(-n3c4ccccc4c4cc(-c5ccc6c(c5)c5ccccc5n6-c5ccc6oc7ccccc7c6c5)ccc43)c2)cc1]

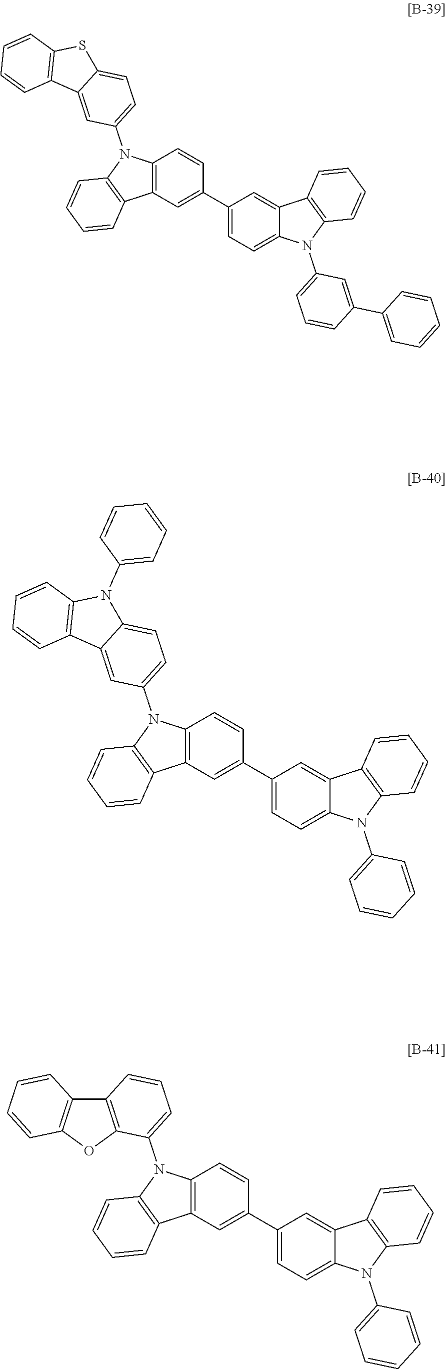 [SMILES: c1ccc(-c2cccc(-n3c4ccccc4c4cc(-c5ccc6c(c5)c5ccccc5n6-c5ccc6sc7ccccc7c6c5)ccc43)c2)cc1 ; c1ccc(-n2c3ccccc3c3cc(-c4ccc5c(c4)c4ccccc4n5-c4ccc5c(c4)c4ccccc4n5-c4ccccc4)ccc32)cc1 ; c1ccc(-n2c3ccccc3c3cc(-c4ccc5c(c4)c4ccccc4n5-c4cccc5c4oc4ccccc45)ccc32)cc1]

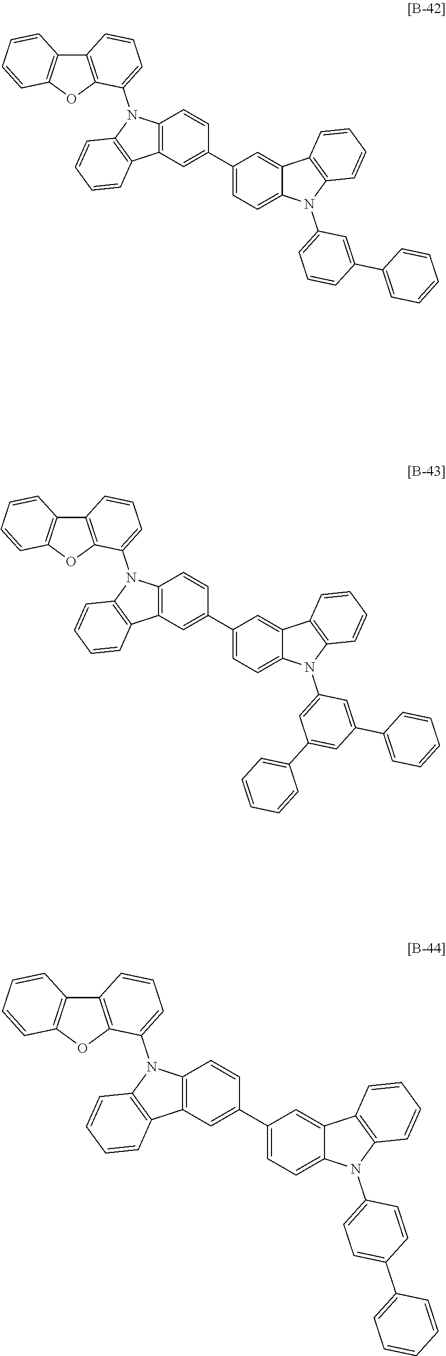 [SMILES: c1ccc(-c2cccc(-n3c4ccccc4c4cc(-c5ccc6c(c5)c5ccccc5n6-c5cccc6c5oc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5oc5ccccc56)ccc43)c2)cc1 ; c1ccc(-c2ccc(-n3c4ccccc4c4cc(-c5ccc6c(c5)c5ccccc5n6-c5cccc6c5oc5ccccc56)ccc43)cc2)cc1]

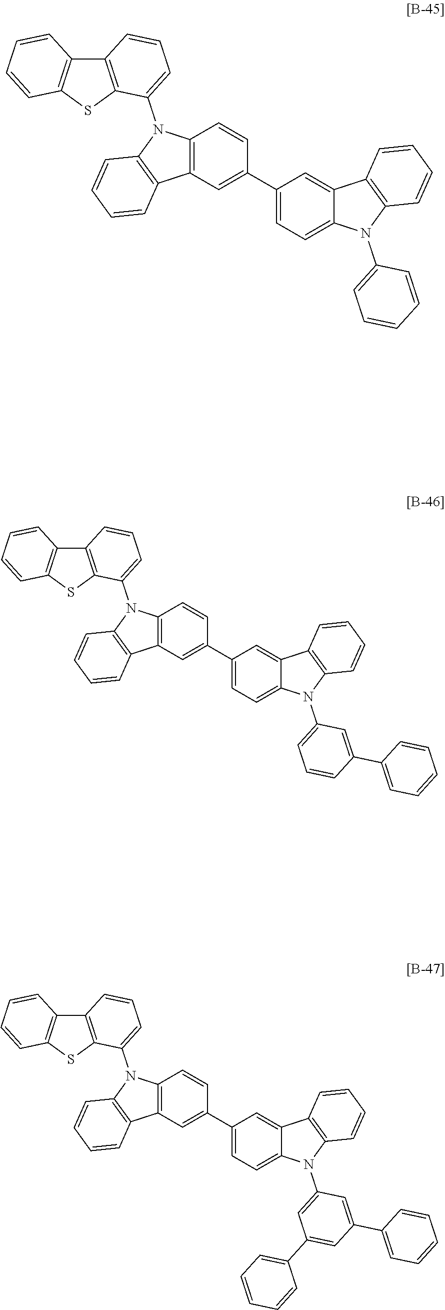 [SMILES: c1ccc(-n2c3ccccc3c3cc(-c4ccc5c(c4)c4ccccc4n5-c4cccc5c4sc4ccccc45)ccc32)cc1 ; c1ccc(-c2cccc(-n3c4ccccc4c4cc(-c5ccc6c(c5)c5ccccc5n6-c5cccc6c5sc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5sc5ccccc56)ccc43)c2)cc1]

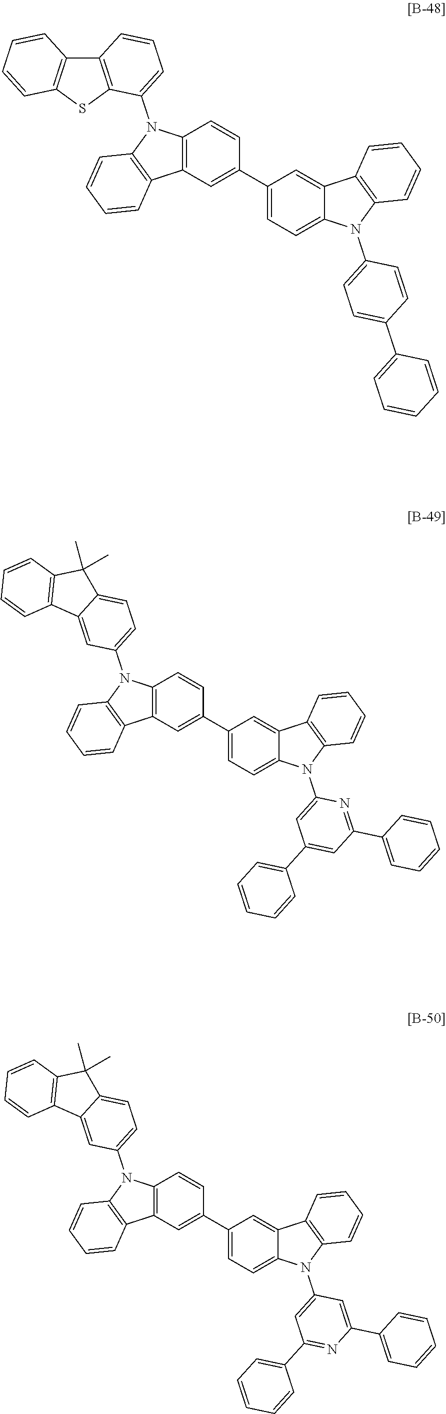 [SMILES: c1ccc(-c2ccc(-n3c4ccccc4c4cc(-c5ccc6c(c5)c5ccccc5n6-c5cccc6c5sc5ccccc56)ccc43)cc2)cc1 ; CC1(C)c2ccccc2-c2cc(-n3c4ccccc4c4cc(-c5ccc6c(c5)c5ccccc5n6-c5cc(-c6ccccc6)cc(-c6ccccc6)n5)ccc43)ccc21 ; CC1(C)c2ccccc2-c2cc(-n3c4ccccc4c4cc(-c5ccc6c(c5)c5ccccc5n6-c5cc(-c6ccccc6)nc(-c6ccccc6)c5)ccc43)ccc21]

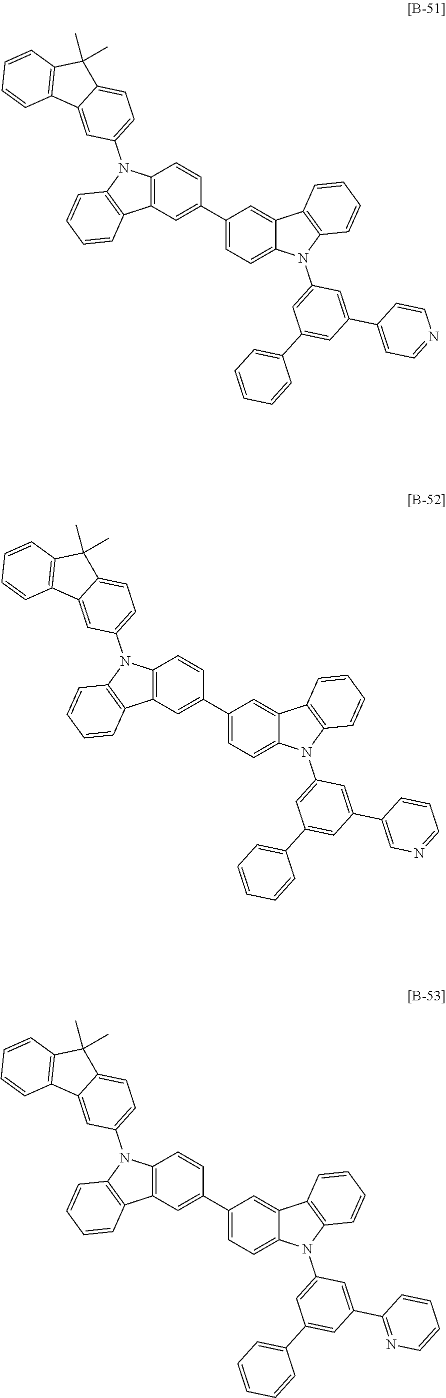 [SMILES: CC1(C)c2ccccc2-c2cc(-n3c4ccccc4c4cc(-c5ccc6c(c5)c5ccccc5n6-c5cc(-c6ccccc6)cc(-c6ccncc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6cccnc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6ccccn6)c5)ccc43)ccc21]

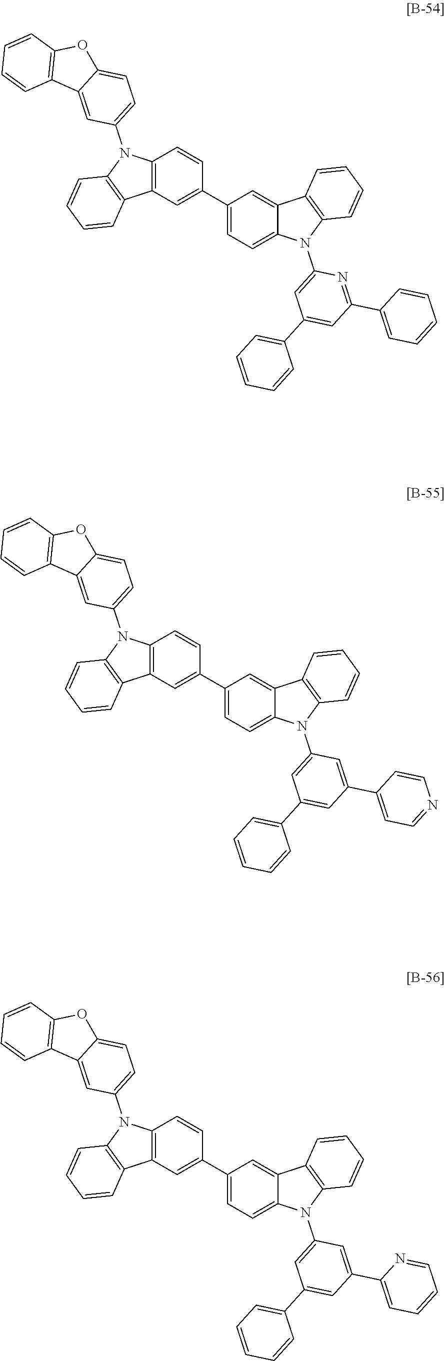 [SMILES: c1ccc(-c2cc(-c3ccccc3)nc(-n3c4ccccc4c4cc(-c5ccc6c(c5)c5ccccc5n6-c5ccc6oc7ccccc7c6c5)ccc43)c2)cc1 ; c1ccc(-c2cc(-c3ccncc3)cc(-n3c4ccccc4c4cc(-c5ccc6c(c5)c5ccccc5n6-c5ccc6oc7ccccc7c6c5)ccc43)c2)cc1 ; c1ccc(-c2cc(-c3ccccn3)cc(-n3c4ccccc4c4cc(-c5ccc6c(c5)c5ccccc5n6-c5ccc6oc7ccccc7c6c5)ccc43)c2)cc1]

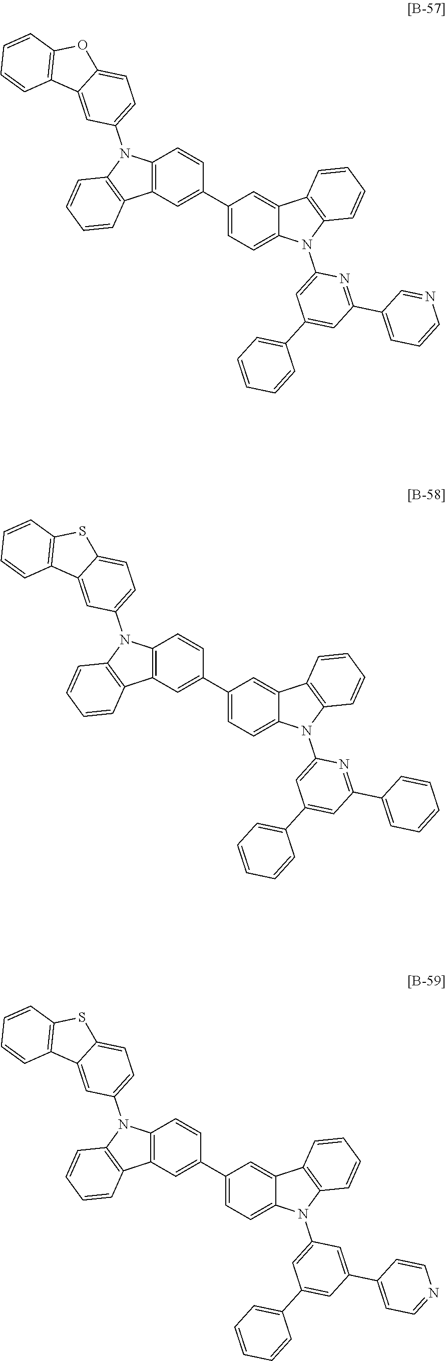 [SMILES: c1ccc(-c2cc(-c3cccnc3)nc(-n3c4ccccc4c4cc(-c5ccc6c(c5)c5ccccc5n6-c5ccc6oc7ccccc7c6c5)ccc43)c2)cc1 ; c1ccc(-c2cc(-c3ccccc3)nc(-n3c4ccccc4c4cc(-c5ccc6c(c5)c5ccccc5n6-c5ccc6sc7ccccc7c6c5)ccc43)c2)cc1 ; c1ccc(-c2cc(-c3ccncc3)cc(-n3c4ccccc4c4cc(-c5ccc6c(c5)c5ccccc5n6-c5ccc6sc7ccccc7c6c5)ccc43)c2)cc1]

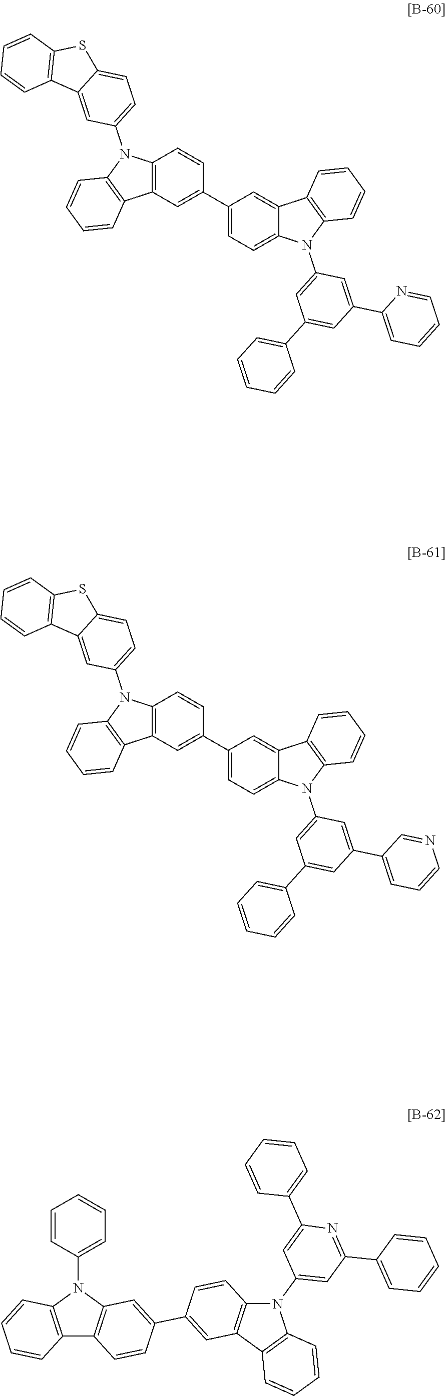 [SMILES: c1ccc(-c2cc(-c3ccccn3)cc(-n3c4ccccc4c4cc(-c5ccc6c(c5)c5ccccc5n6-c5ccc6sc7ccccc7c6c5)ccc43)c2)cc1 ; c1ccc(-c2cc(-c3cccnc3)cc(-n3c4ccccc4c4cc(-c5ccc6c(c5)c5ccccc5n6-c5ccc6sc7ccccc7c6c5)ccc43)c2)cc1 ; c1ccc(-c2cc(-n3c4ccccc4c4cc(-c5ccc6c7ccccc7n(-c7ccccc7)c6c5)ccc43)cc(-c3ccccc3)n2)cc1]

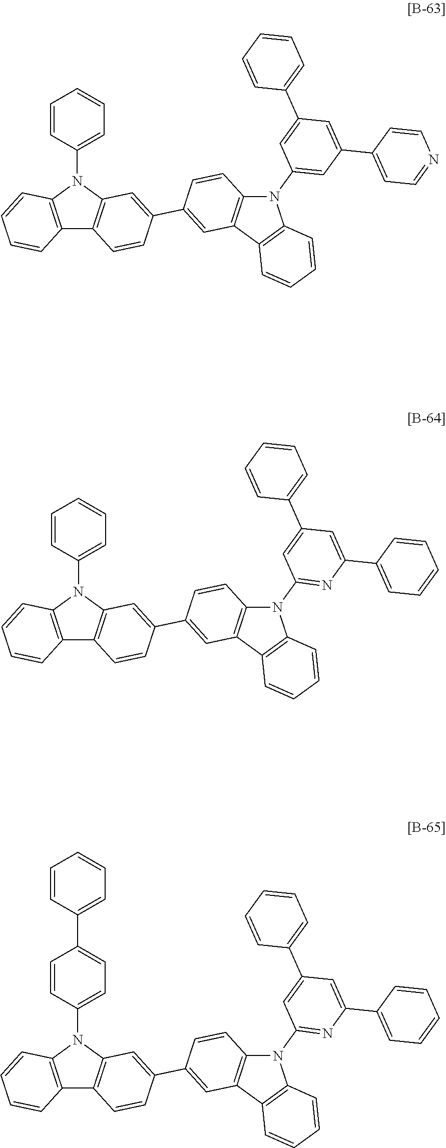 [SMILES: c1ccc(-c2cc(-c3ccncc3)cc(-n3c4ccccc4c4cc(-c5ccc6c7ccccc7n(-c7ccccc7)c6c5)ccc43)c2)cc1 ; c1ccc(-c2cc(-c3ccccc3)nc(-n3c4ccccc4c4cc(-c5ccc6c7ccccc7n(-c7ccccc7)c6c5)ccc43)c2)cc1 ; c1ccc(-c2ccc(-n3c4ccccc4c4ccc(-c5ccc6c(c5)c5ccccc5n6-c5cc(-c6ccccc6)cc(-c6ccccc6)n5)cc43)cc2)cc1]

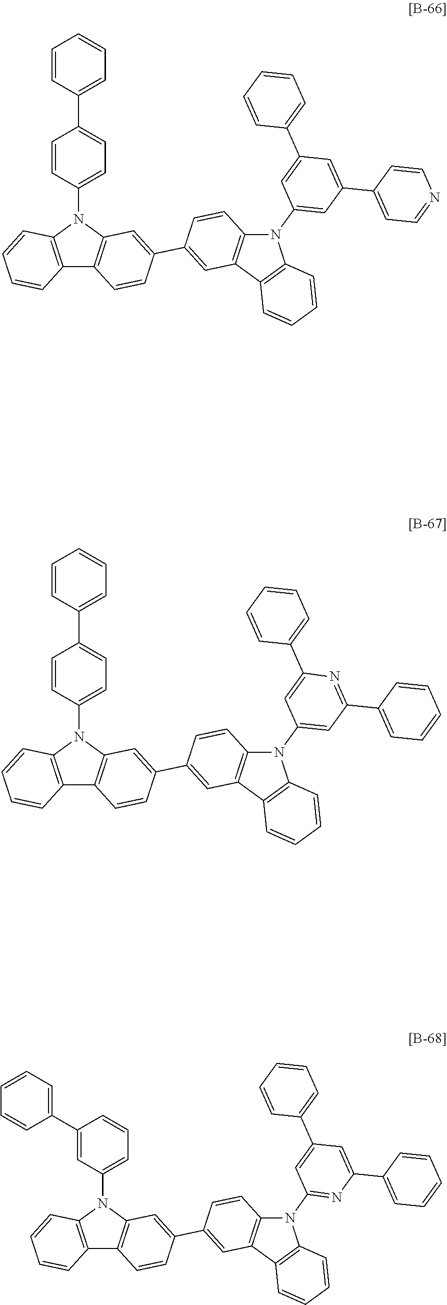 [SMILES: c1ccc(-c2ccc(-n3c4ccccc4c4ccc(-c5ccc6c(c5)c5ccccc5n6-c5cc(-c6ccccc6)cc(-c6ccncc6)c5)cc43)cc2)cc1 ; c1ccc(-c2ccc(-n3c4ccccc4c4ccc(-c5ccc6c(c5)c5ccccc5n6-c5cc(-c6ccccc6)nc(-c6ccccc6)c5)cc43)cc2)cc1 ; c1ccc(-c2cccc(-n3c4ccccc4c4ccc(-c5ccc6c(c5)c5ccccc5n6-c5cc(-c6ccccc6)cc(-c6ccccc6)n5)cc43)c2)cc1]

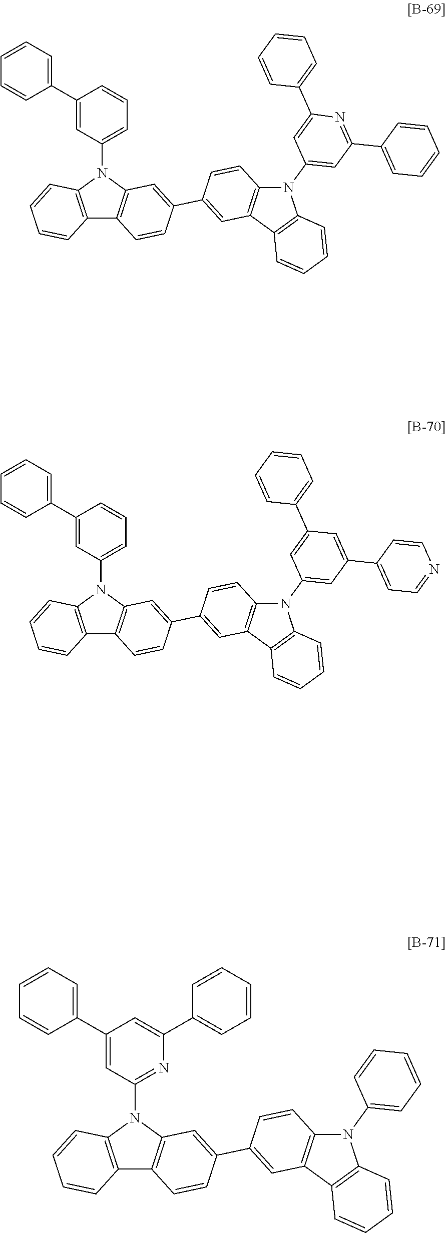 [SMILES: c1ccc(-c2cccc(-n3c4ccccc4c4ccc(-c5ccc6c(c5)c5ccccc5n6-c5cc(-c6ccccc6)nc(-c6ccccc6)c5)cc43)c2)cc1 ; c1ccc(-c2cccc(-n3c4ccccc4c4ccc(-c5ccc6c(c5)c5ccccc5n6-c5cc(-c6ccccc6)cc(-c6ccncc6)c5)cc43)c2)cc1 ; c1ccc(-c2cc(-c3ccccc3)nc(-n3c4ccccc4c4ccc(-c5ccc6c(c5)c5ccccc5n6-c5ccccc5)cc43)c2)cc1]

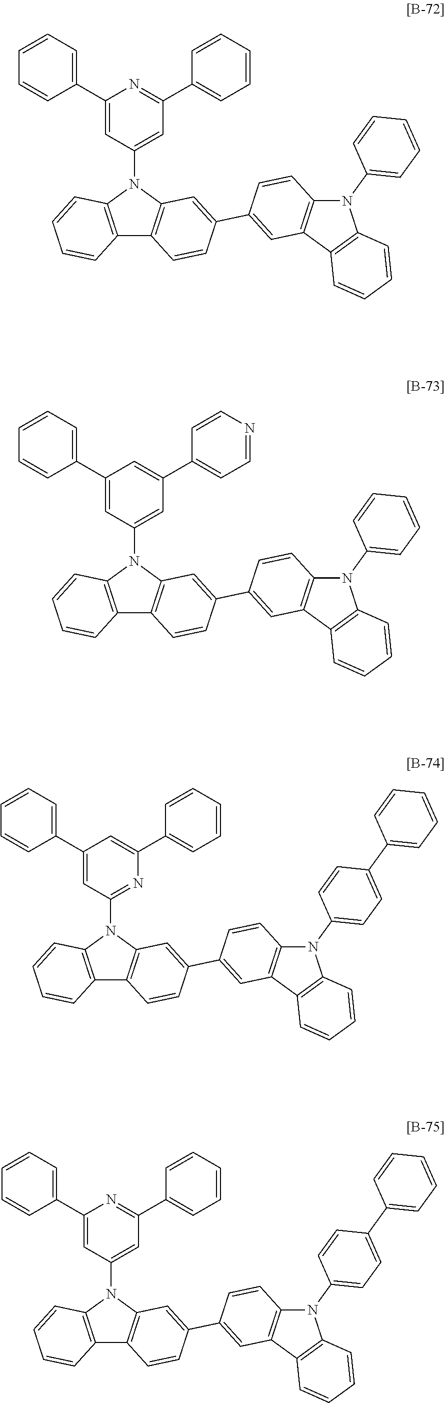 [SMILES: c1ccc(-c2cc(-n3c4ccccc4c4ccc(-c5ccc6c(c5)c5ccccc5n6-c5ccccc5)cc43)cc(-c3ccccc3)n2)cc1 ; c1ccc(-c2cc(-c3ccncc3)cc(-n3c4ccccc4c4ccc(-c5ccc6c(c5)c5ccccc5n6-c5ccccc5)cc43)c2)cc1 ; c1ccc(-c2ccc(-n3c4ccccc4c4cc(-c5ccc6c7ccccc7n(-c7cc(-c8ccccc8)cc(-c8ccccc8)n7)c6c5)ccc43)cc2)cc1 ; c1ccc(-c2ccc(-n3c4ccccc4c4cc(-c5ccc6c7ccccc7n(-c7cc(-c8ccccc8)nc(-c8ccccc8)c7)c6c5)ccc43)cc2)cc1]

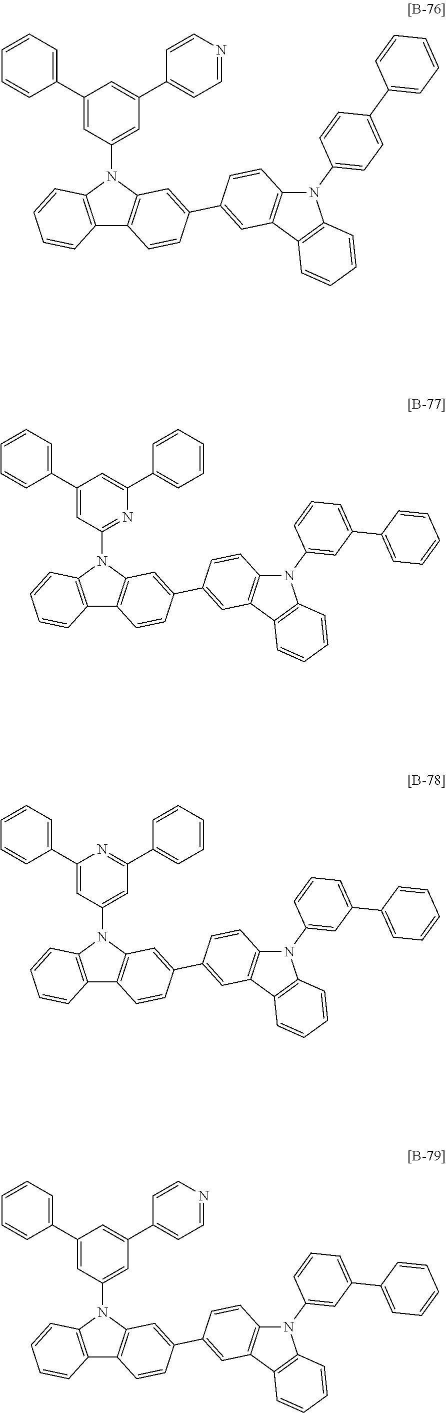 [SMILES: c1ccc(-c2ccc(-n3c4ccccc4c4cc(-c5ccc6c7ccccc7n(-c7cc(-c8ccccc8)cc(-c8ccncc8)c7)c6c5)ccc43)cc2)cc1 ; c1ccc(-c2cccc(-n3c4ccccc4c4cc(-c5ccc6c7ccccc7n(-c7cc(-c8ccccc8)cc(-c8ccccc8)n7)c6c5)ccc43)c2)cc1 ; c1ccc(-c2cccc(-n3c4ccccc4c4cc(-c5ccc6c7ccccc7n(-c7cc(-c8ccccc8)nc(-c8ccccc8)c7)c6c5)ccc43)c2)cc1 ; c1ccc(-c2cccc(-n3c4ccccc4c4cc(-c5ccc6c7ccccc7n(-c7cc(-c8ccccc8)cc(-c8ccncc8)c7)c6c5)ccc43)c2)cc1]

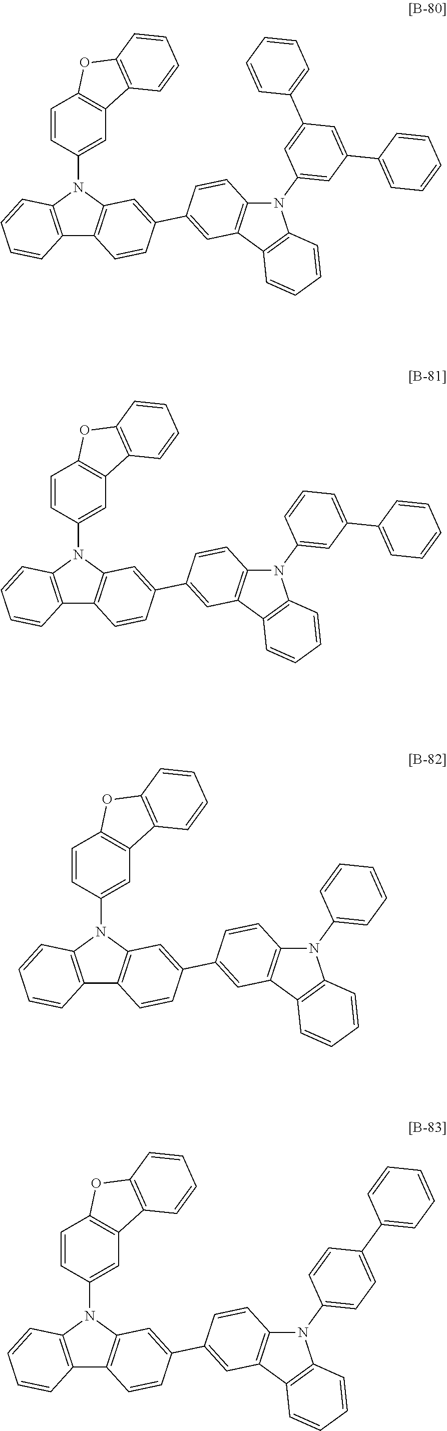 [SMILES: c1ccc(-c2cc(-c3ccccc3)cc(-n3c4ccccc4c4cc(-c5ccc6c7ccccc7n(-c7ccc8oc9ccccc9c8c7)c6c5)ccc43)c2)cc1 ; c1ccc(-c2cccc(-n3c4ccccc4c4cc(-c5ccc6c7ccccc7n(-c7ccc8oc9ccccc9c8c7)c6c5)ccc43)c2)cc1 ; c1ccc(-n2c3ccccc3c3cc(-c4ccc5c6ccccc6n(-c6ccc7oc8ccccc8c7c6)c5c4)ccc32)cc1 ; c1ccc(-c2ccc(-n3c4ccccc4c4cc(-c5ccc6c7ccccc7n(-c7ccc8oc9ccccc9c8c7)c6c5)ccc43)cc2)cc1]

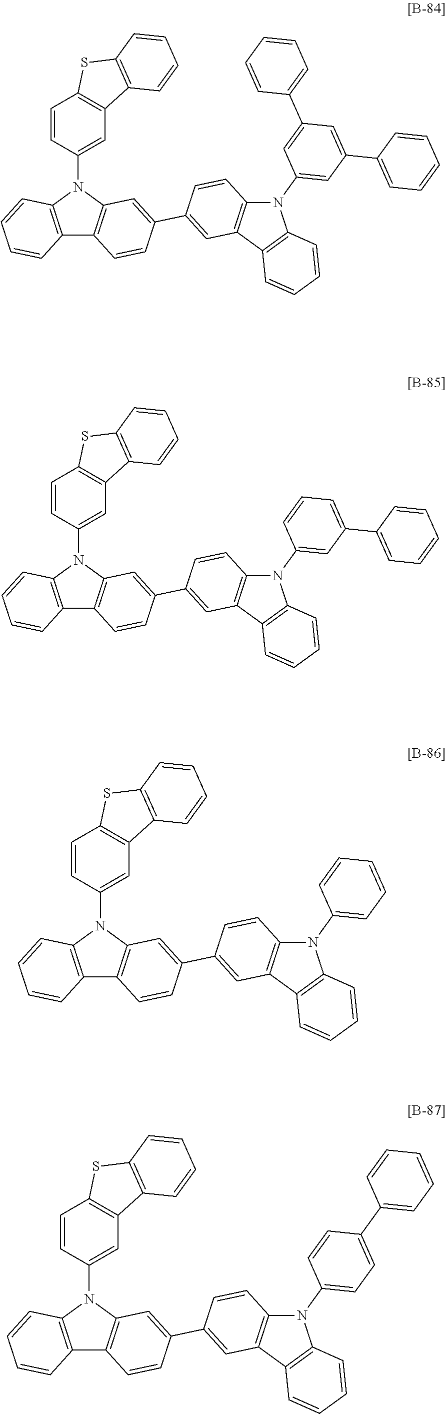 [SMILES: c1ccc(-c2cc(-c3ccccc3)cc(-n3c4ccccc4c4cc(-c5ccc6c7ccccc7n(-c7ccc8sc9ccccc9c8c7)c6c5)ccc43)c2)cc1 ; c1ccc(-c2cccc(-n3c4ccccc4c4cc(-c5ccc6c7ccccc7n(-c7ccc8sc9ccccc9c8c7)c6c5)ccc43)c2)cc1 ; c1ccc(-n2c3ccccc3c3cc(-c4ccc5c6ccccc6n(-c6ccc7sc8ccccc8c7c6)c5c4)ccc32)cc1 ; c1ccc(-c2ccc(-n3c4ccccc4c4cc(-c5ccc6c7ccccc7n(-c7ccc8sc9ccccc9c8c7)c6c5)ccc43)cc2)cc1]

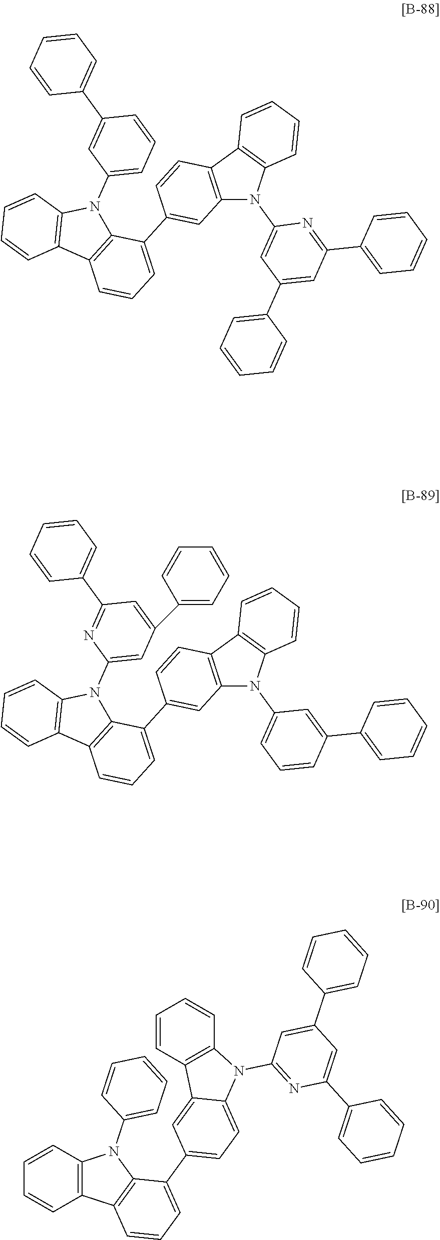 [SMILES: c1ccc(-c2cccc(-n3c4ccccc4c4cccc(-c5ccc6c7ccccc7n(-c7cc(-c8ccccc8)cc(-c8ccccc8)n7)c6c5)c43)c2)cc1 ; c1ccc(-c2cccc(-n3c4ccccc4c4ccc(-c5cccc6c7ccccc7n(-c7cc(-c8ccccc8)cc(-c8ccccc8)n7)c56)cc43)c2)cc1 ; c1ccc(-c2cc(-c3ccccc3)nc(-n3c4ccccc4c4cc(-c5cccc6c7ccccc7n(-c7ccccc7)c56)ccc43)c2)cc1]

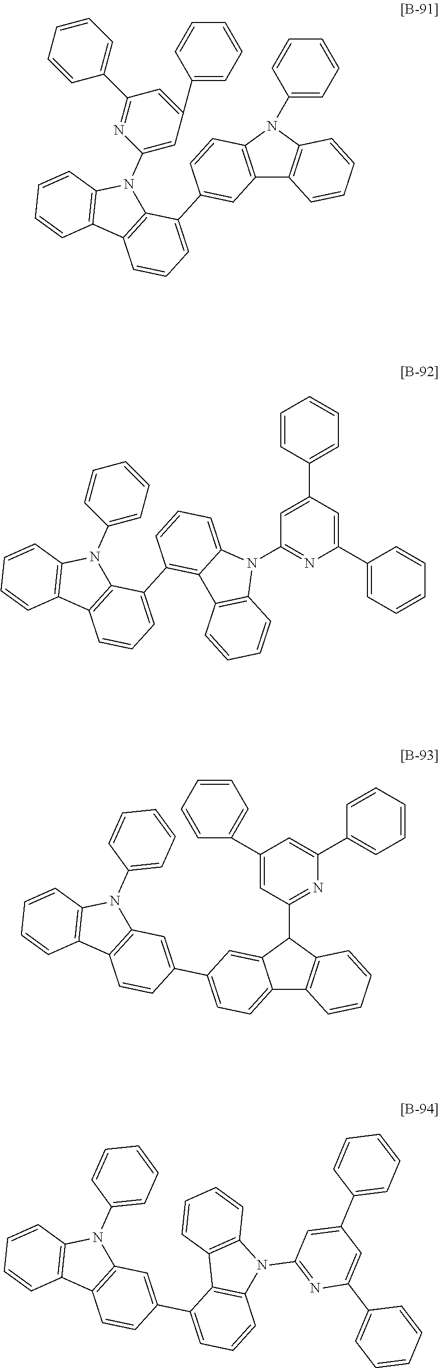 [SMILES: c1ccc(-c2cc(-c3ccccc3)nc(-n3c4ccccc4c4cccc(-c5ccc6c(c5)c5ccccc5n6-c5ccccc5)c43)c2)cc1 ; c1ccc(-c2cc(-c3ccccc3)nc(-n3c4ccccc4c4c(-c5cccc6c7ccccc7n(-c7ccccc7)c56)cccc43)c2)cc1 ; c1ccc(-c2cc(-c3ccccc3)nc(C3c4ccccc4-c4ccc(-c5ccc6c7ccccc7n(-c7ccccc7)c6c5)cc43)c2)cc1 ; c1ccc(-c2cc(-c3ccccc3)nc(-n3c4ccccc4c4c(-c5ccc6c7ccccc7n(-c7ccccc7)c6c5)cccc43)c2)cc1]

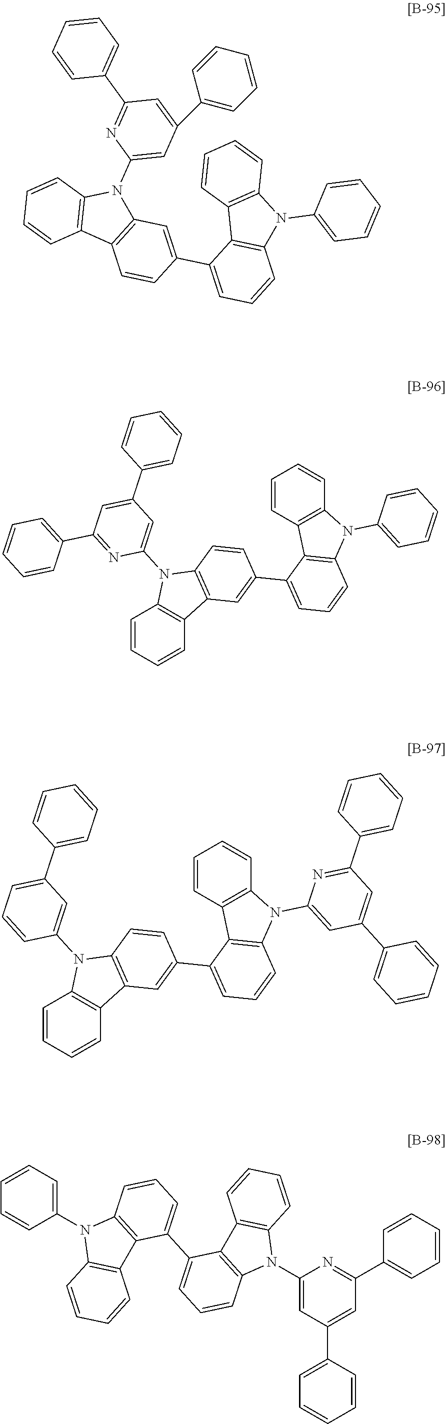 [SMILES: c1ccc(-c2cc(-c3ccccc3)nc(-n3c4ccccc4c4ccc(-c5cccc6c5c5ccccc5n6-c5ccccc5)cc43)c2)cc1 ; c1ccc(-c2cc(-c3ccccc3)nc(-n3c4ccccc4c4cc(-c5cccc6c5c5ccccc5n6-c5ccccc5)ccc43)c2)cc1 ; c1ccc(-c2cccc(-n3c4ccccc4c4cc(-c5cccc6c5c5ccccc5n6-c5cc(-c6ccccc6)cc(-c6ccccc6)n5)ccc43)c2)cc1 ; c1ccc(-c2cc(-c3ccccc3)nc(-n3c4ccccc4c4c(-c5cccc6c5c5ccccc5n6-c5ccccc5)cccc43)c2)cc1]

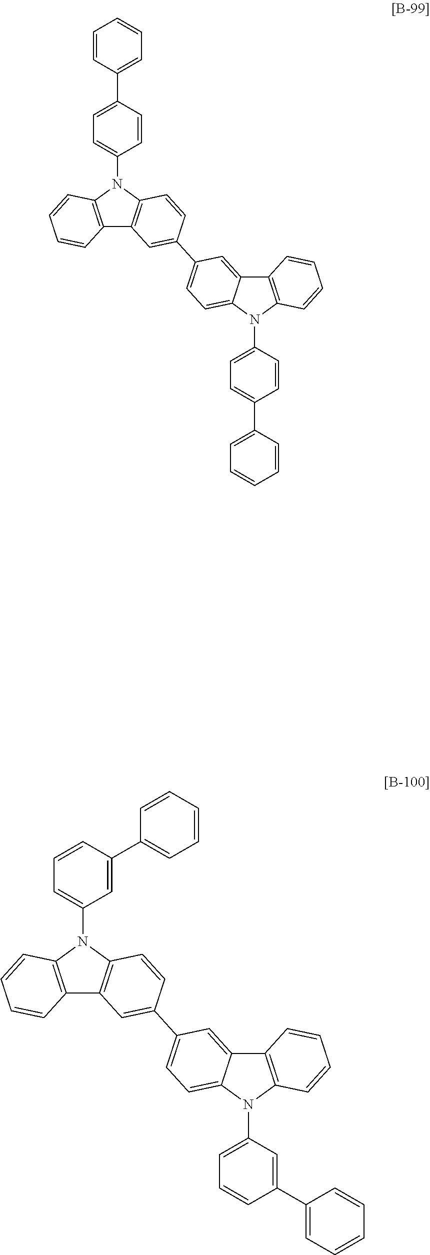 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

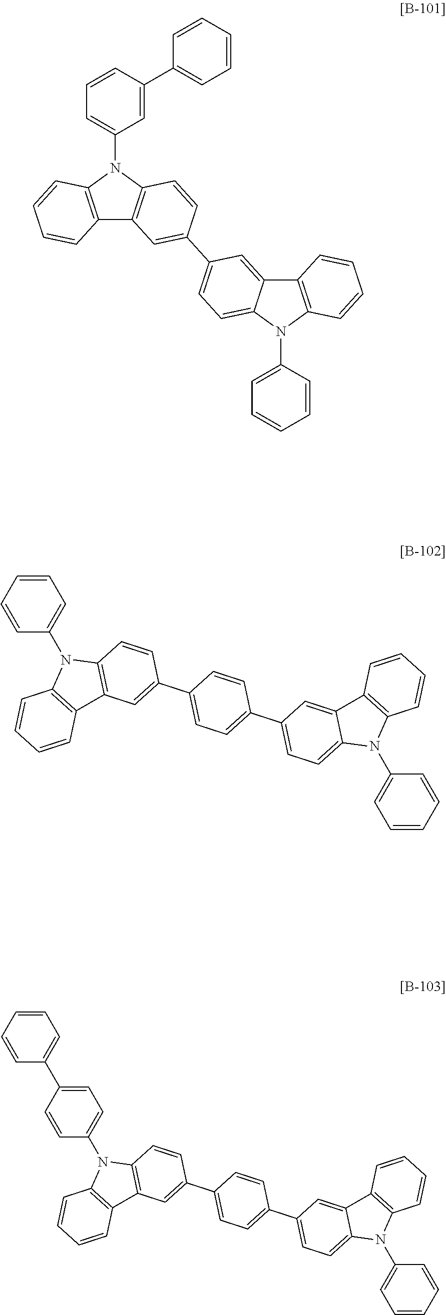 [SMILES: c1ccc(-c2cccc(-n3c4ccccc4c4cc(-c5ccc6c(c5)c5ccccc5n6-c5ccccc5)ccc43)c2)cc1 ; c1ccc(-n2c3ccccc3c3cc(-c4ccc(-c5ccc6c(c5)c5ccccc5n6-c5ccccc5)cc4)ccc32)cc1 ; c1ccc(-c2ccc(-n3c4ccccc4c4cc(-c5ccc(-c6ccc7c(c6)c6ccccc6n7-c6ccccc6)cc5)ccc43)cc2)cc1]

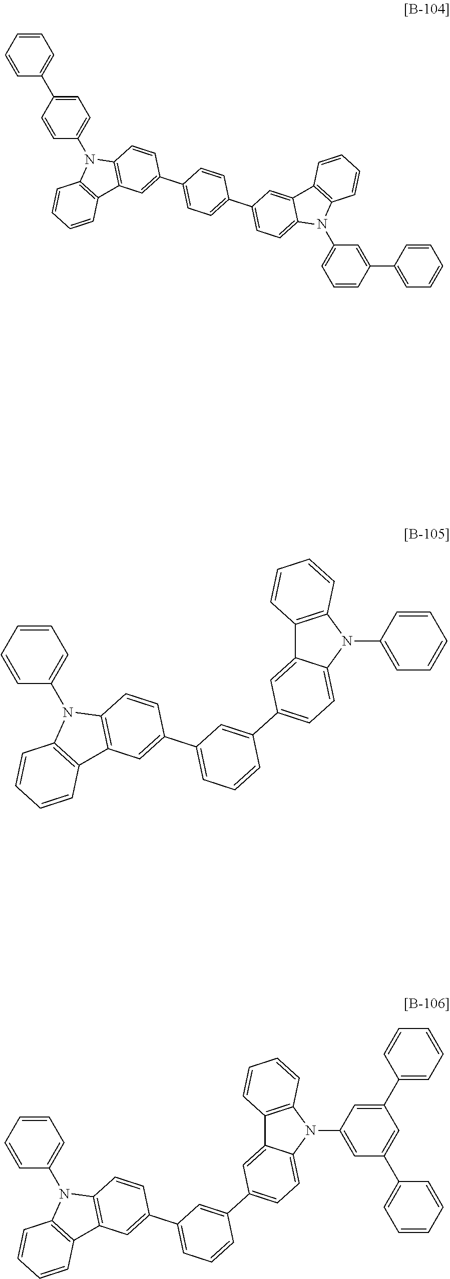 [SMILES: c1ccc(-c2ccc(-n3c4ccccc4c4cc(-c5ccc(-c6ccc7c(c6)c6ccccc6n7-c6cccc(-c7ccccc7)c6)cc5)ccc43)cc2)cc1 ; c1ccc(-n2c3ccccc3c3cc(-c4cccc(-c5ccc6c(c5)c5ccccc5n6-c5ccccc5)c4)ccc32)cc1 ; c1ccc(-c2cc(-c3ccccc3)cc(-n3c4ccccc4c4cc(-c5cccc(-c6ccc7c(c6)c6ccccc6n7-c6ccccc6)c5)ccc43)c2)cc1]

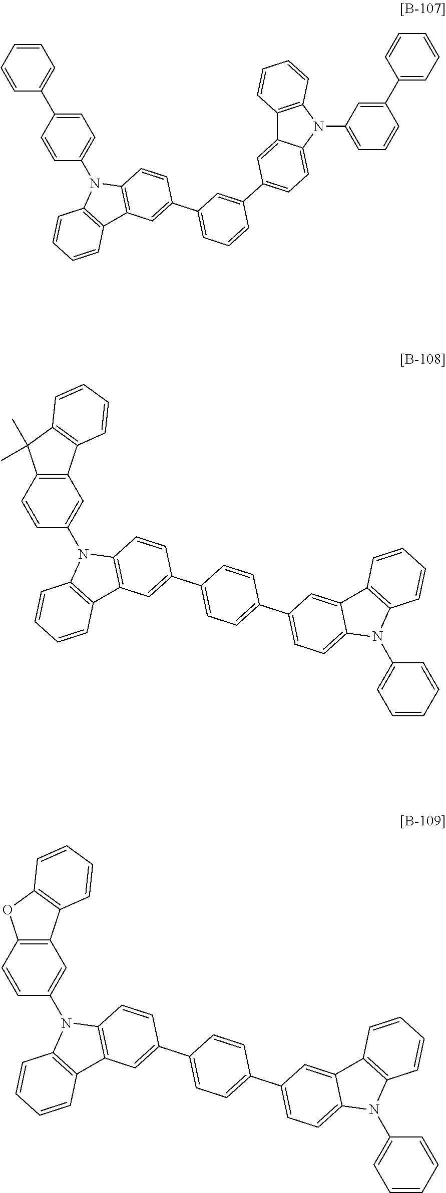 [SMILES: c1ccc(-c2ccc(-n3c4ccccc4c4cc(-c5cccc(-c6ccc7c(c6)c6ccccc6n7-c6cccc(-c7ccccc7)c6)c5)ccc43)cc2)cc1 ; CC1(C)c2ccccc2-c2cc(-n3c4ccccc4c4cc(-c5ccc(-c6ccc7c(c6)c6ccccc6n7-c6ccccc6)cc5)ccc43)ccc21 ; c1ccc(-n2c3ccccc3c3cc(-c4ccc(-c5ccc6c(c5)c5ccccc5n6-c5ccc6oc7ccccc7c6c5)cc4)ccc32)cc1]

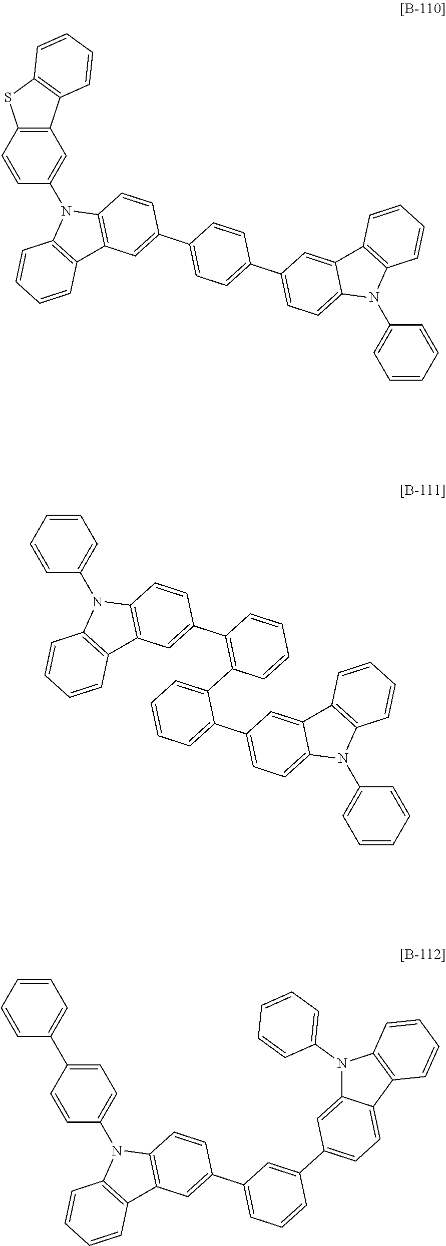 [SMILES: c1ccc(-n2c3ccccc3c3cc(-c4ccc(-c5ccc6c(c5)c5ccccc5n6-c5ccc6sc7ccccc7c6c5)cc4)ccc32)cc1 ; c1ccc(-n2c3ccccc3c3cc(-c4ccccc4-c4ccccc4-c4ccc5c(c4)c4ccccc4n5-c4ccccc4)ccc32)cc1 ; c1ccc(-c2ccc(-n3c4ccccc4c4cc(-c5cccc(-c6ccc7c8ccccc8n(-c8ccccc8)c7c6)c5)ccc43)cc2)cc1]

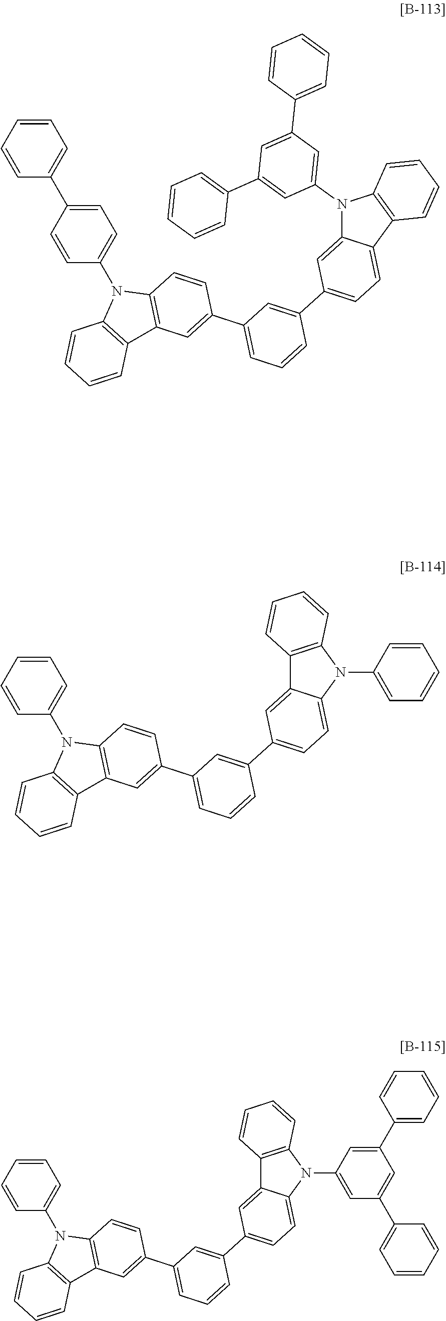 [SMILES: c1ccc(-c2ccc(-n3c4ccccc4c4cc(-c5cccc(-c6ccc7c8ccccc8n(-c8cc(-c9ccccc9)cc(-c9ccccc9)c8)c7c6)c5)ccc43)cc2)cc1 ; c1ccc(-n2c3ccccc3c3cc(-c4cccc(-c5ccc6c(c5)c5ccccc5n6-c5ccccc5)c4)ccc32)cc1 ; c1ccc(-c2cc(-c3ccccc3)cc(-n3c4ccccc4c4cc(-c5cccc(-c6ccc7c(c6)c6ccccc6n7-c6ccccc6)c5)ccc43)c2)cc1]

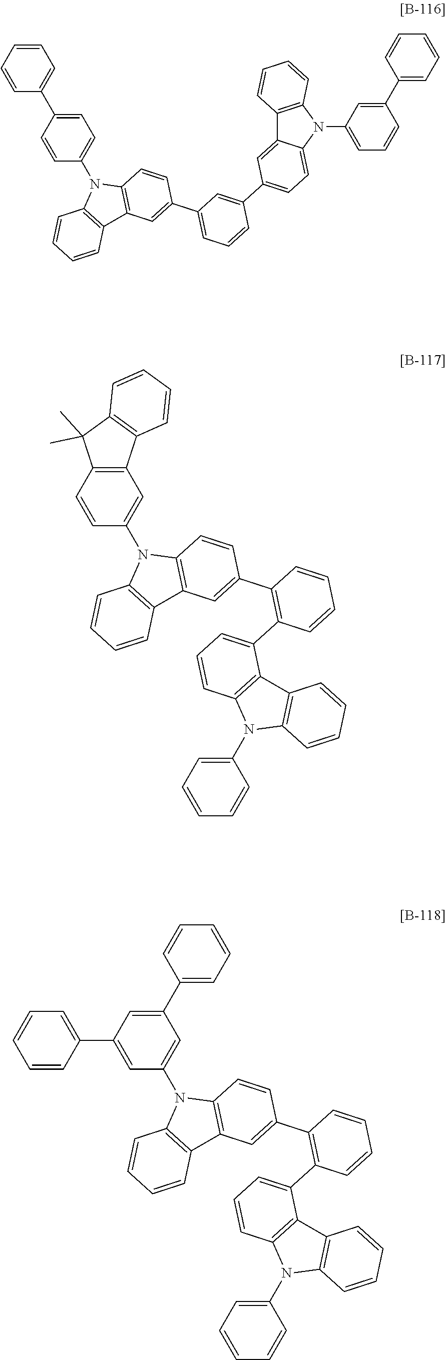 [SMILES: c1ccc(-c2ccc(-n3c4ccccc4c4cc(-c5cccc(-c6ccc7c(c6)c6ccccc6n7-c6cccc(-c7ccccc7)c6)c5)ccc43)cc2)cc1 ; CC1(C)c2ccccc2-c2cc(-n3c4ccccc4c4cc(-c5ccccc5-c5cccc6c5c5ccccc5n6-c5ccccc5)ccc43)ccc21 ; c1ccc(-c2cc(-c3ccccc3)cc(-n3c4ccccc4c4cc(-c5ccccc5-c5cccc6c5c5ccccc5n6-c5ccccc5)ccc43)c2)cc1]

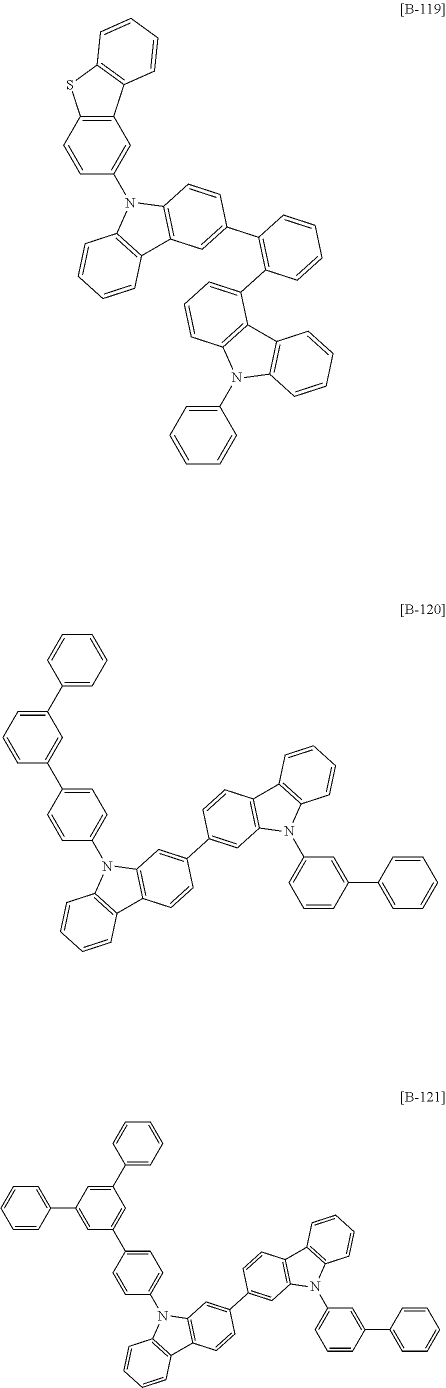 [SMILES: c1ccc(-n2c3ccccc3c3c(-c4ccccc4-c4ccc5c(c4)c4ccccc4n5-c4ccc5sc6ccccc6c5c4)cccc32)cc1 ; c1ccc(-c2cccc(-c3ccc(-n4c5ccccc5c5ccc(-c6ccc7c8ccccc8n(-c8cccc(-c9ccccc9)c8)c7c6)cc54)cc3)c2)cc1 ; c1ccc(-c2cc(-c3ccccc3)cc(-c3ccc(-n4c5ccccc5c5ccc(-c6ccc7c8ccccc8n(-c8cccc(-c9ccccc9)c8)c7c6)cc54)cc3)c2)cc1]

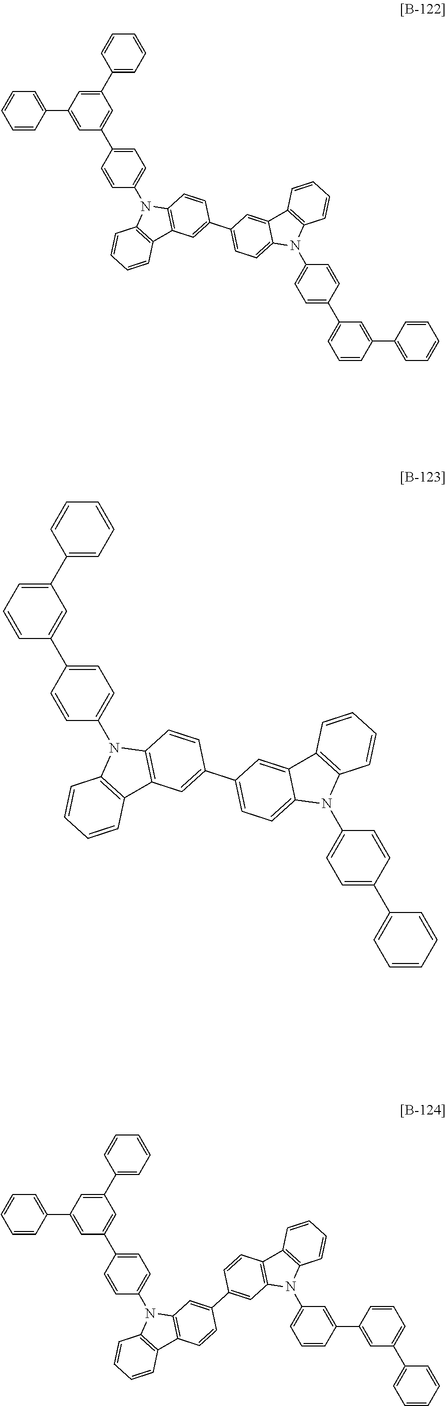 [SMILES: c1ccc(-c2cccc(-c3ccc(-n4c5ccccc5c5cc(-c6ccc7c(c6)c6ccccc6n7-c6ccc(-c7cc(-c8ccccc8)cc(-c8ccccc8)c7)cc6)ccc54)cc3)c2)cc1 ; c1ccc(-c2ccc(-n3c4ccccc4c4cc(-c5ccc6c(c5)c5ccccc5n6-c5ccc(-c6cccc(-c7ccccc7)c6)cc5)ccc43)cc2)cc1 ; c1ccc(-c2cccc(-c3cccc(-n4c5ccccc5c5ccc(-c6ccc7c8ccccc8n(-c8ccc(-c9cc(-c%10ccccc%10)cc(-c%10ccccc%10)c9)cc8)c7c6)cc54)c3)c2)cc1]

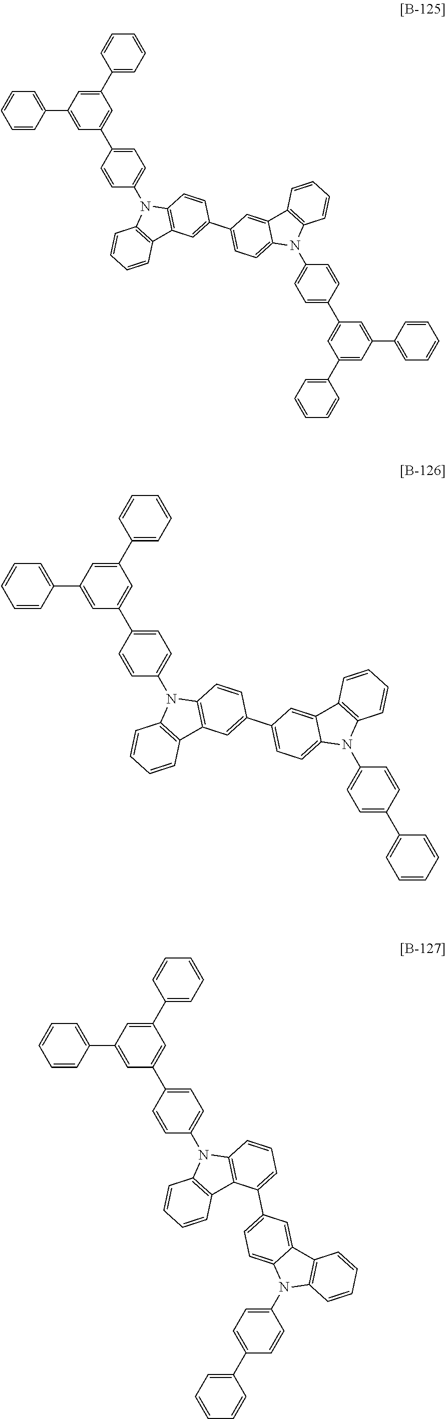 [SMILES: c1ccc(-c2cc(-c3ccccc3)cc(-c3ccc(-n4c5ccccc5c5cc(-c6ccc7c(c6)c6ccccc6n7-c6ccc(-c7cc(-c8ccccc8)cc(-c8ccccc8)c7)cc6)ccc54)cc3)c2)cc1 ; c1ccc(-c2ccc(-n3c4ccccc4c4cc(-c5ccc6c(c5)c5ccccc5n6-c5ccc(-c6cc(-c7ccccc7)cc(-c7ccccc7)c6)cc5)ccc43)cc2)cc1 ; c1ccc(-c2ccc(-n3c4ccccc4c4cc(-c5cccc6c5c5ccccc5n6-c5ccc(-c6cc(-c7ccccc7)cc(-c7ccccc7)c6)cc5)ccc43)cc2)cc1]

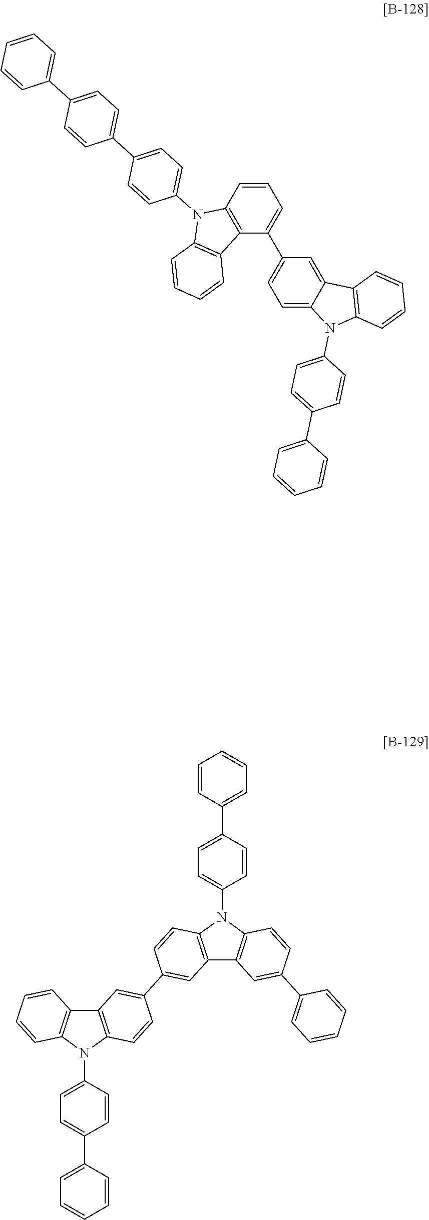 [SMILES: c1ccc(-c2ccc(-c3ccc(-n4c5ccccc5c5c(-c6ccc7c(c6)c6ccccc6n7-c6ccc(-c7ccccc7)cc6)cccc54)cc3)cc2)cc1 ; c1ccc(-c2ccc(-n3c4ccccc4c4cc(-c5ccc6c(c5)c5cc(-c7ccccc7)ccc5n6-c5ccc(-c6ccccc6)cc5)ccc43)cc2)cc1]

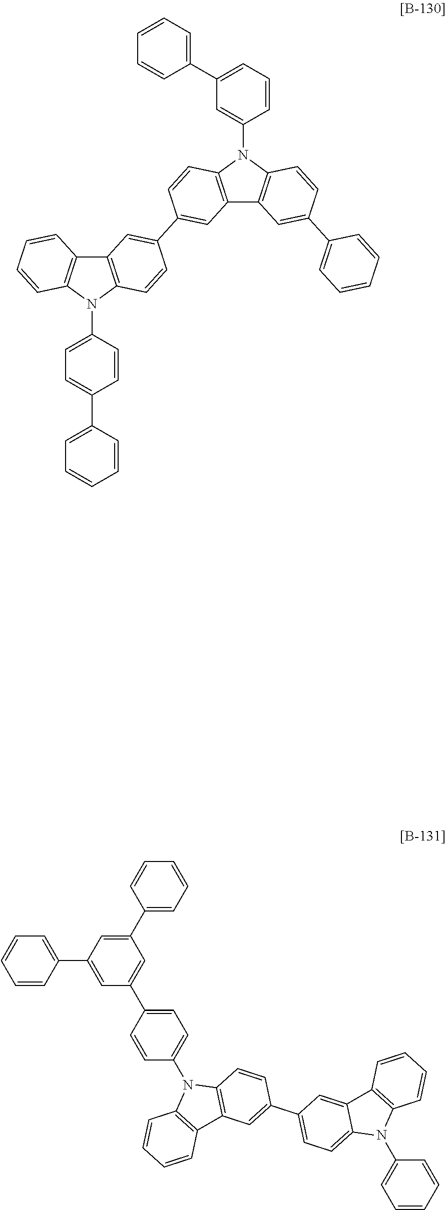 [SMILES: c1ccc(-c2ccc(-n3c4ccccc4c4cc(-c5ccc6c(c5)c5cc(-c7ccccc7)ccc5n6-c5cccc(-c6ccccc6)c5)ccc43)cc2)cc1 ; c1ccc(-c2cc(-c3ccccc3)cc(-c3ccc(-n4c5ccccc5c5cc(-c6ccc7c(c6)c6ccccc6n7-c6ccccc6)ccc54)cc3)c2)cc1]

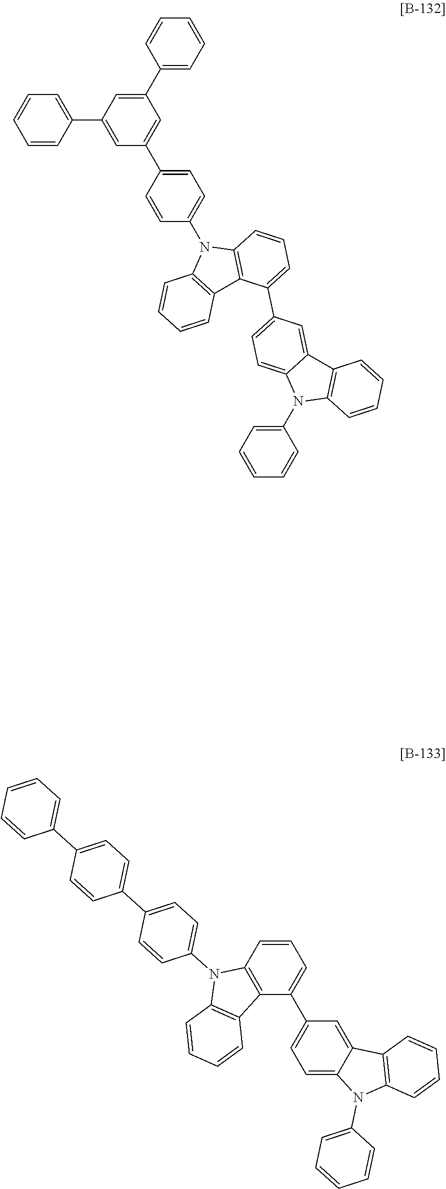 [SMILES: c1ccc(-c2cc(-c3ccccc3)cc(-c3ccc(-n4c5ccccc5c5c(-c6ccc7c(c6)c6ccccc6n7-c6ccccc6)cccc54)cc3)c2)cc1 ; c1ccc(-c2ccc(-c3ccc(-n4c5ccccc5c5c(-c6ccc7c(c6)c6ccccc6n7-c6ccccc6)cccc54)cc3)cc2)cc1]

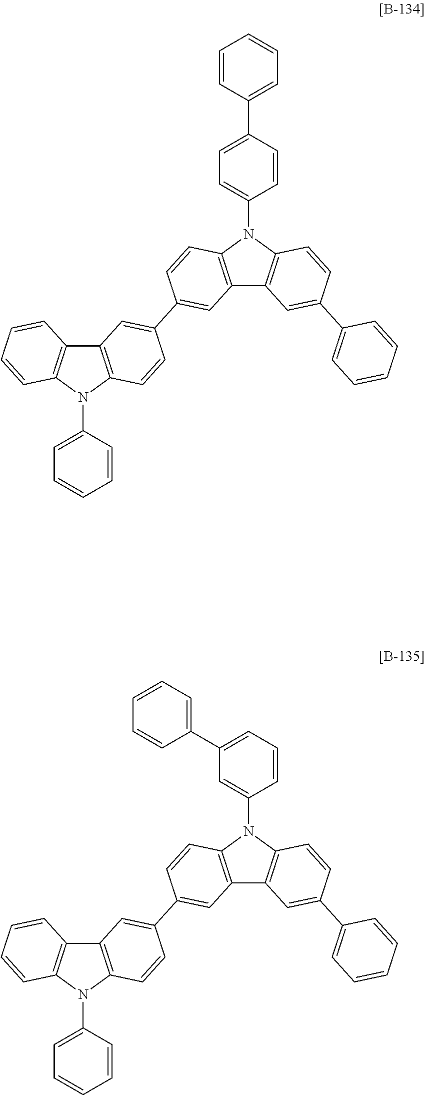 [SMILES: c1ccc(-c2ccc(-n3c4ccc(-c5ccccc5)cc4c4cc(-c5ccc6c(c5)c5ccccc5n6-c5ccccc5)ccc43)cc2)cc1 ; c1ccc(-c2cccc(-n3c4ccc(-c5ccccc5)cc4c4cc(-c5ccc6c(c5)c5ccccc5n6-c5ccccc5)ccc43)c2)cc1]

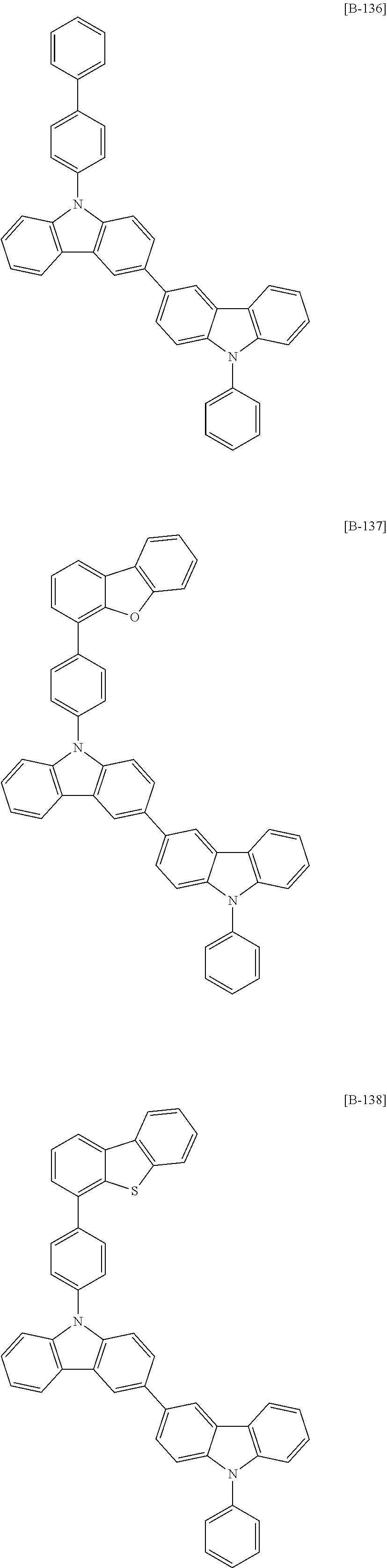 [SMILES: c1ccc(-c2ccc(-n3c4ccccc4c4cc(-c5ccc6c(c5)c5ccccc5n6-c5ccccc5)ccc43)cc2)cc1 ; c1ccc(-n2c3ccccc3c3cc(-c4ccc5c(c4)c4ccccc4n5-c4ccc(-c5cccc6c5oc5ccccc56)cc4)ccc32)cc1 ; c1ccc(-n2c3ccccc3c3cc(-c4ccc5c(c4)c4ccccc4n5-c4ccc(-c5cccc6c5sc5ccccc56)cc4)ccc32)cc1]

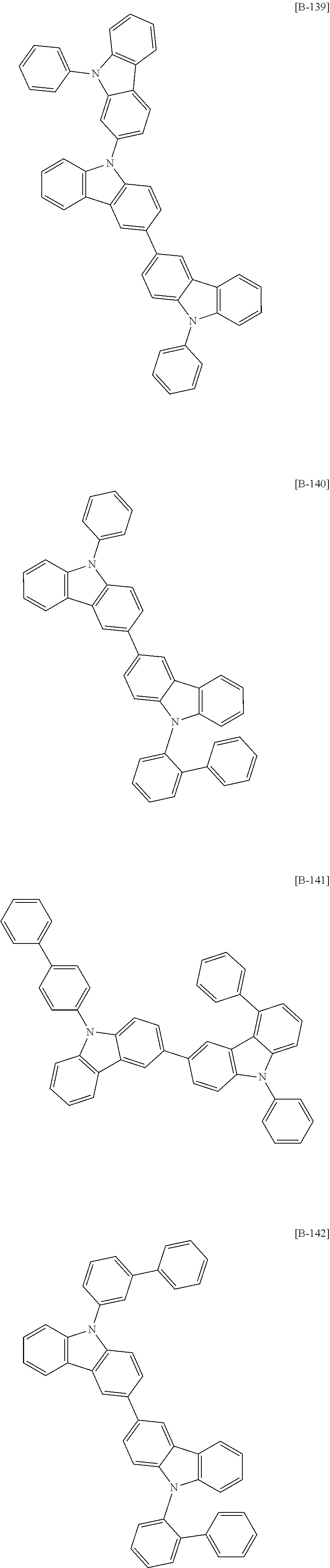 [SMILES: c1ccc(-n2c3ccccc3c3cc(-c4ccc5c(c4)c4ccccc4n5-c4ccc5c6ccccc6n(-c6ccccc6)c5c4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5c(-c7ccccc7)cccc5n6-c5ccccc5)ccc43)cc2)cc1 ; c1ccc(-c2cccc(-n3c4ccccc4c4cc(-c5ccc6c(c5)c5ccccc5n6-c5ccccc5-c5ccccc5)ccc43)c2)cc1]

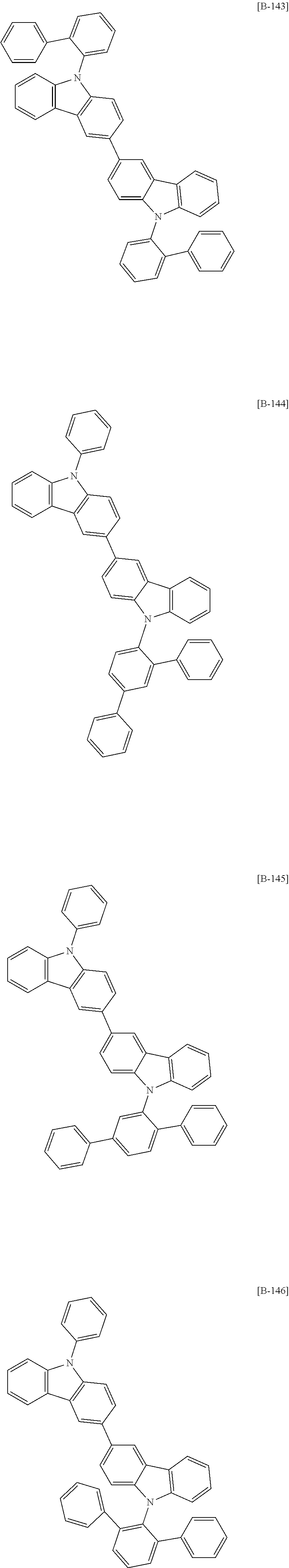 [SMILES: c1ccc(-c2ccccc2-n2c3ccccc3c3cc(-c4ccc5c(c4)c4ccccc4n5-c4ccccc4-c4ccccc4)ccc32)cc1 ; c1ccc(-c2ccc(-n3c4ccccc4c4cc(-c5ccc6c(c5)c5ccccc5n6-c5ccccc5)ccc43)c(-c3ccccc3)c2)cc1 ; c1ccc(-c2ccc(-c3ccccc3)c(-n3c4ccccc4c4cc(-c5ccc6c(c5)c5ccccc5n6-c5ccccc5)ccc43)c2)cc1 ; c1ccc(-c2cccc(-c3ccccc3)c2-n2c3ccccc3c3cc(-c4ccc5c(c4)c4ccccc4n5-c4ccccc4)ccc32)cc1]

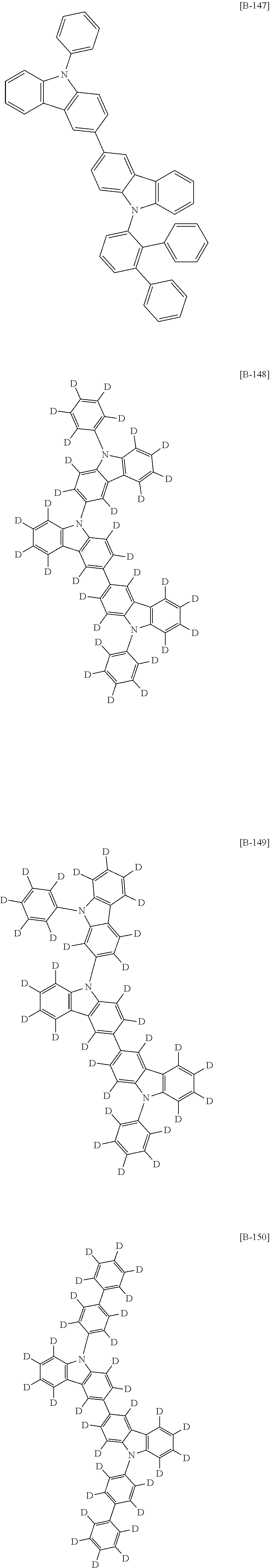

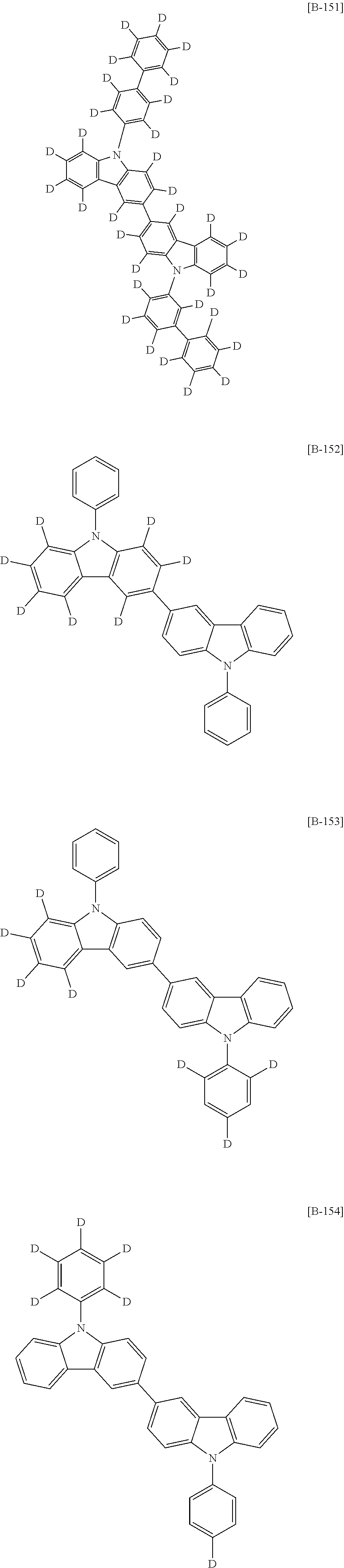

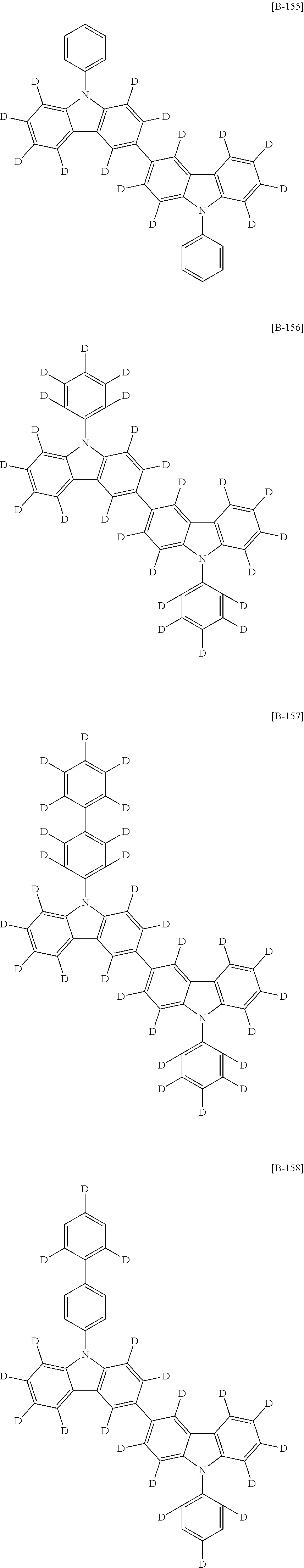

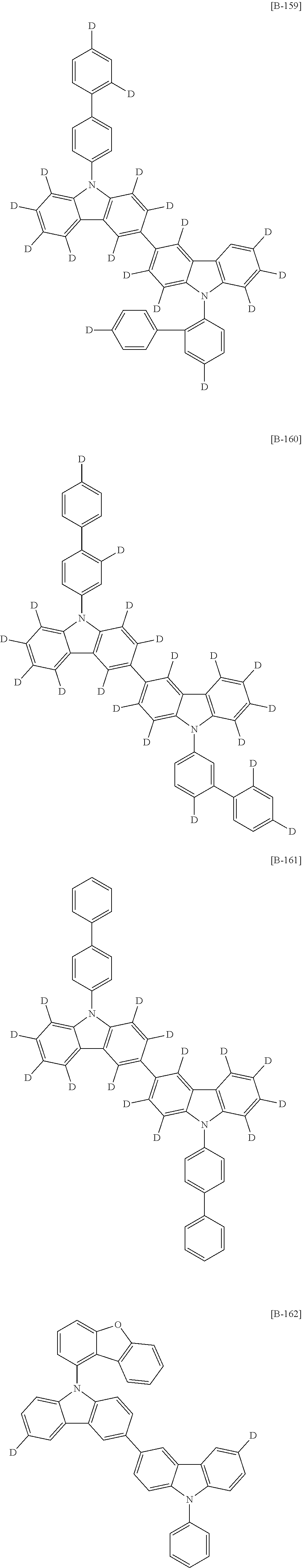

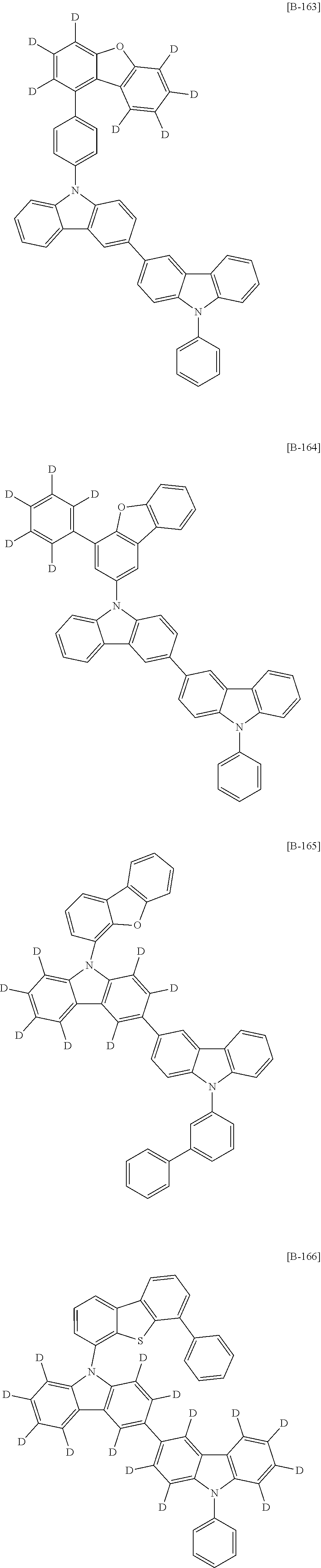

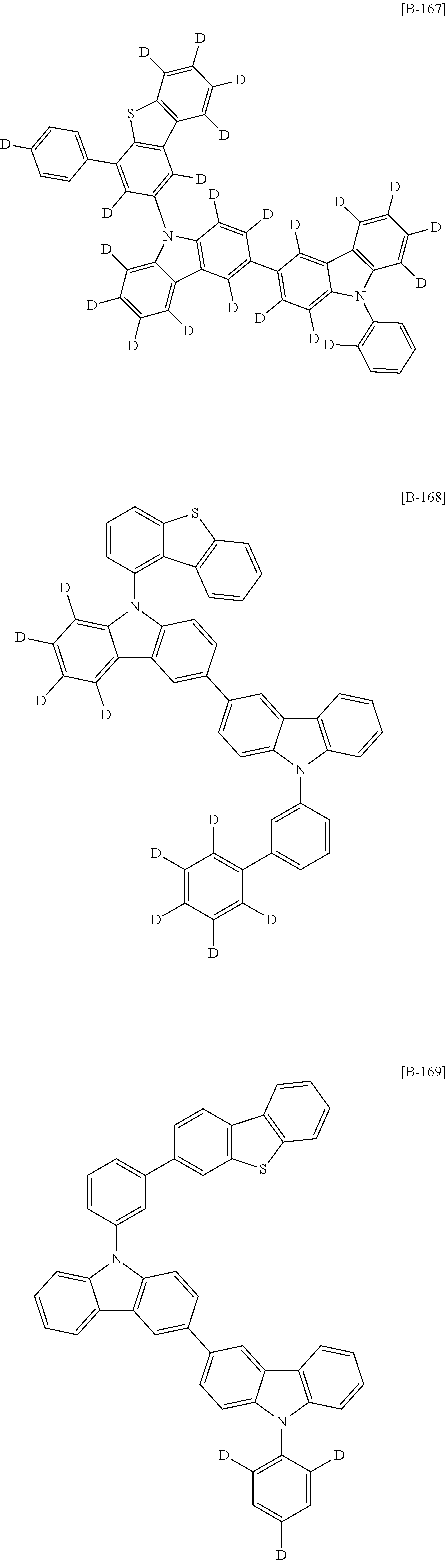 [SMILES: [2H]c1ccc(-c2c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccccc5[2H])c([2H])c([2H])c43)c([2H])c3c2sc2c([2H])c([2H])c([2H])c([2H])c23)cc1 ; [2H]c1c([2H])c([2H])c(-c2cccc(-n3c4ccccc4c4cc(-c5ccc6c(c5)c5c([2H])c([2H])c([2H])c([2H])c5n6-c5cccc6sc7ccccc7c56)ccc43)c2)c([2H])c1[2H] ; [2H]c1cc([2H])c(-n2c3ccccc3c3cc(-c4ccc5c(c4)c4ccccc4n5-c4cccc(-c5ccc6c(c5)sc5ccccc56)c4)ccc32)c([2H])c1]

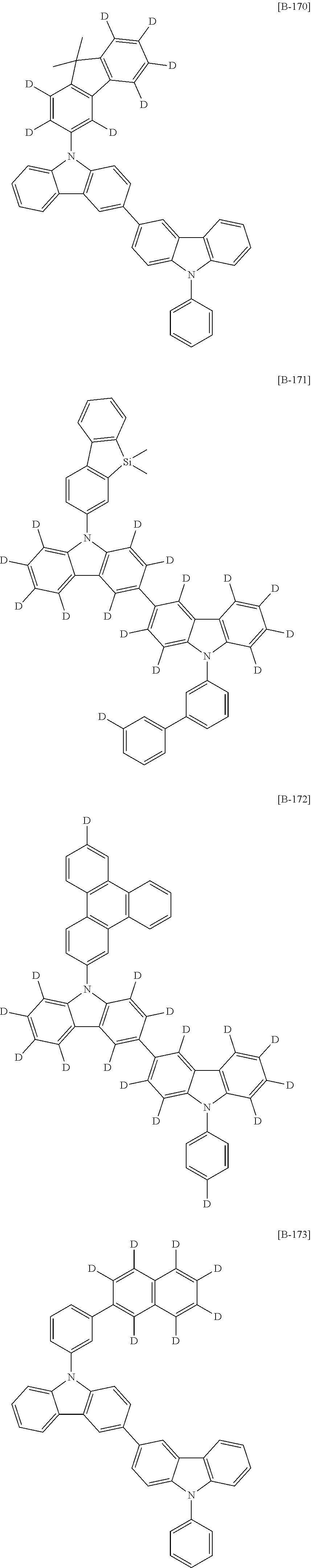

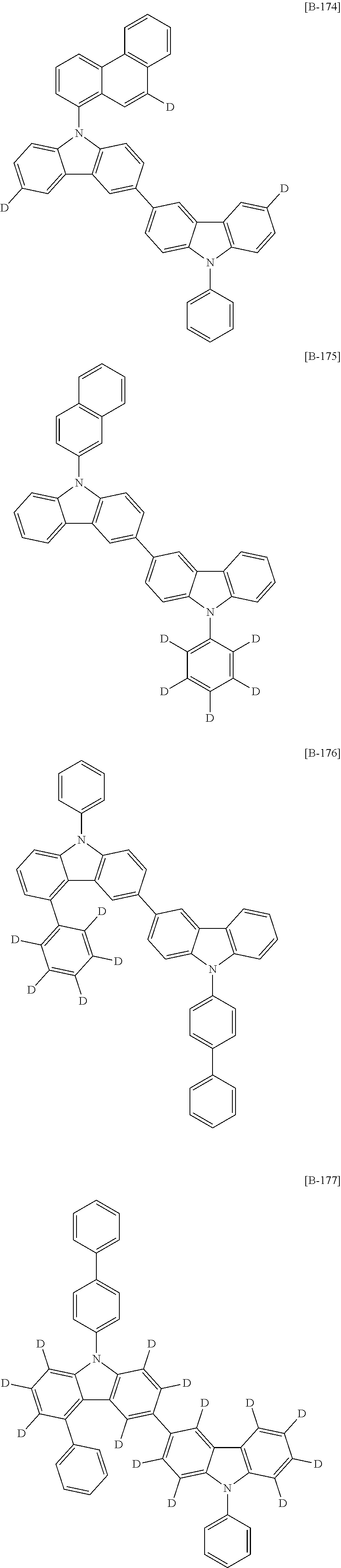 [SMILES: [2H]c1ccc2c(c1)c1cc(-c3ccc4c(c3)c3cc([2H])ccc3n4-c3cccc4c3cc([2H])c3ccccc34)ccc1n2-c1ccccc1 ; [2H]c1c([2H])c([2H])c(-n2c3ccccc3c3cc(-c4ccc5c(c4)c4ccccc4n5-c4ccc5ccccc5c4)ccc32)c([2H])c1[2H] ; [2H]c1c([2H])c([2H])c(-c2cccc3c2c2cc(-c4ccc5c(c4)c4ccccc4n5-c4ccc(-c5ccccc5)cc4)ccc2n3-c2ccccc2)c([2H])c1[2H] ; [2H]c1c([2H])c([2H])c2c(c1[2H])c1c([2H])c(-c3c([2H])c([2H])c4c(c3[2H])c3c(-c5ccccc5)c([2H])c([2H])c([2H])c3n4-c3ccc(-c4ccccc4)cc3)c([2H])c([2H])c1n2-c1ccccc1]

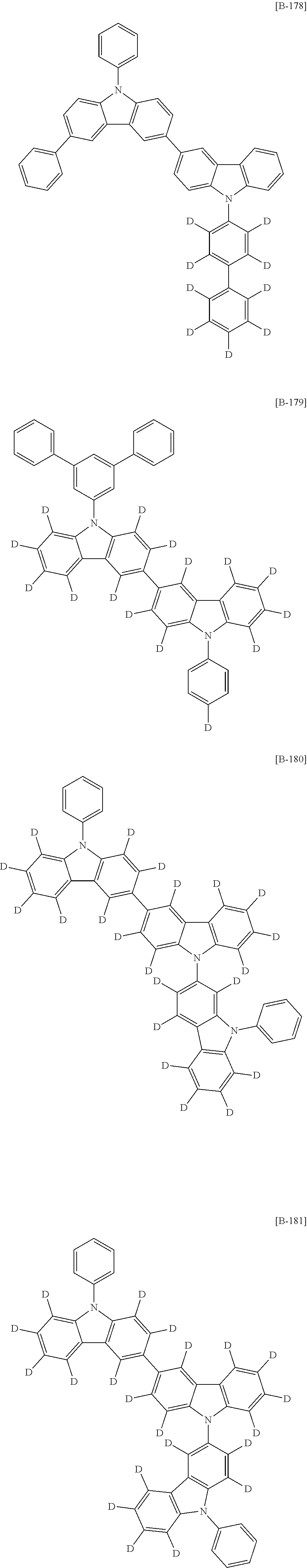

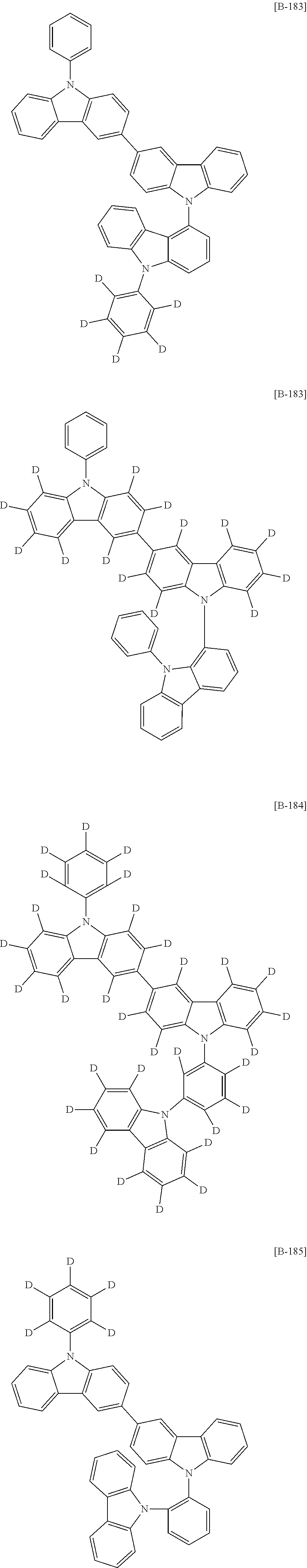

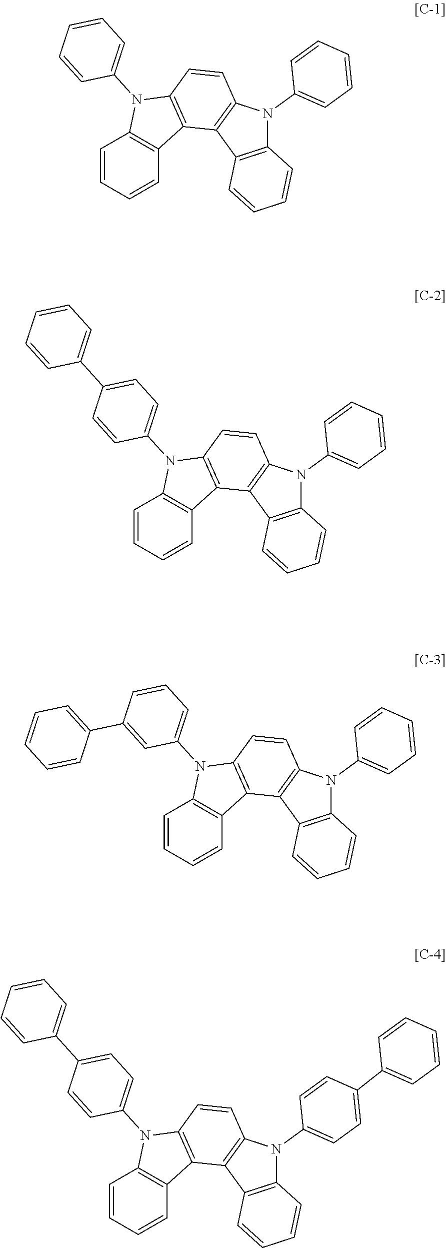 [SMILES: c1ccc(-n2c3ccccc3c3c4c5ccccc5n(-c5ccccc5)c4ccc32)cc1 ; c1ccc(-c2ccc(-n3c4ccccc4c4c5c6ccccc6n(-c6ccccc6)c5ccc43)cc2)cc1 ; c1ccc(-c2cccc(-n3c4ccccc4c4c5c6ccccc6n(-c6ccccc6)c5ccc43)c2)cc1 ; c1ccc(-c2ccc(-n3c4ccccc4c4c5c6ccccc6n(-c6ccc(-c7ccccc7)cc6)c5ccc43)cc2)cc1]

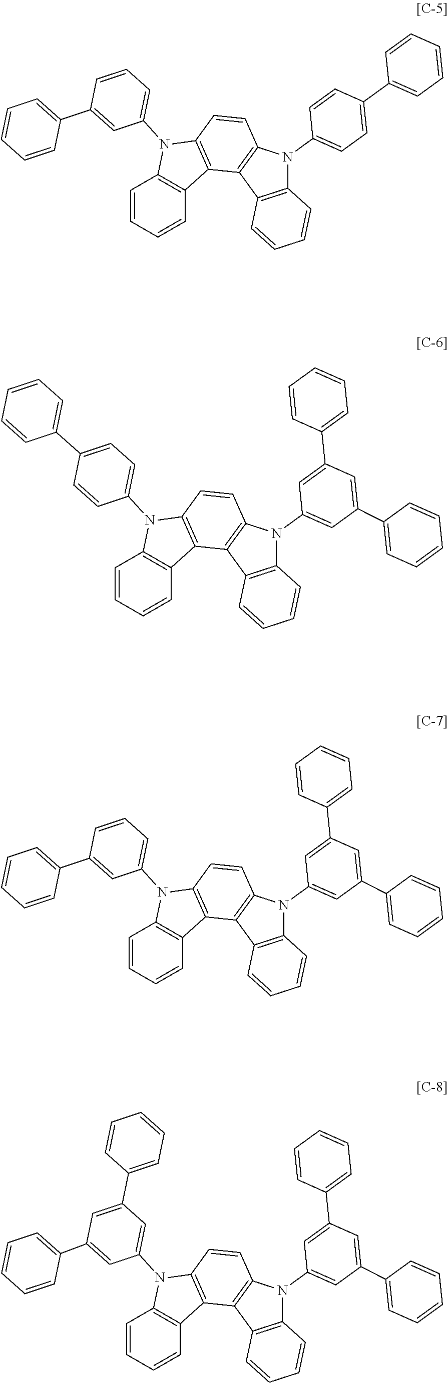 [SMILES: c1ccc(-c2ccc(-n3c4ccccc4c4c5c6ccccc6n(-c6cccc(-c7ccccc7)c6)c5ccc43)cc2)cc1 ; c1ccc(-c2ccc(-n3c4ccccc4c4c5c6ccccc6n(-c6cc(-c7ccccc7)cc(-c7ccccc7)c6)c5ccc43)cc2)cc1 ; c1ccc(-c2cccc(-n3c4ccccc4c4c5c6ccccc6n(-c6cc(-c7ccccc7)cc(-c7ccccc7)c6)c5ccc43)c2)cc1 ; c1ccc(-c2cc(-c3ccccc3)cc(-n3c4ccccc4c4c5c6ccccc6n(-c6cc(-c7ccccc7)cc(-c7ccccc7)c6)c5ccc43)c2)cc1]

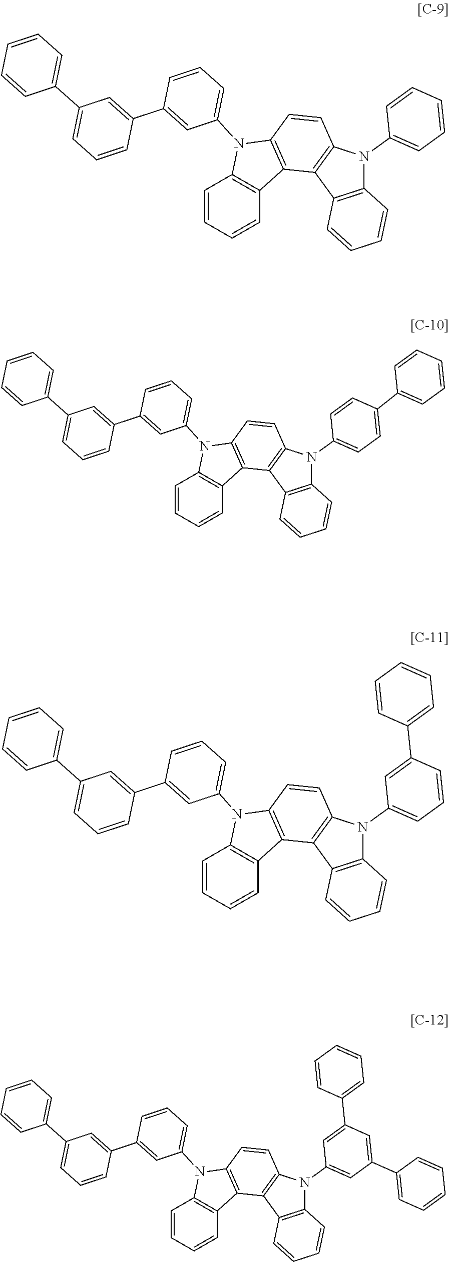 [SMILES: c1ccc(-c2cccc(-c3cccc(-n4c5ccccc5c5c6c7ccccc7n(-c7ccccc7)c6ccc54)c3)c2)cc1 ; c1ccc(-c2ccc(-n3c4ccccc4c4c5c6ccccc6n(-c6cccc(-c7cccc(-c8ccccc8)c7)c6)c5ccc43)cc2)cc1 ; c1ccc(-c2cccc(-c3cccc(-n4c5ccccc5c5c6c7ccccc7n(-c7cccc(-c8ccccc8)c7)c6ccc54)c3)c2)cc1 ; c1ccc(-c2cccc(-c3cccc(-n4c5ccccc5c5c6c7ccccc7n(-c7cc(-c8ccccc8)cc(-c8ccccc8)c7)c6ccc54)c3)c2)cc1]

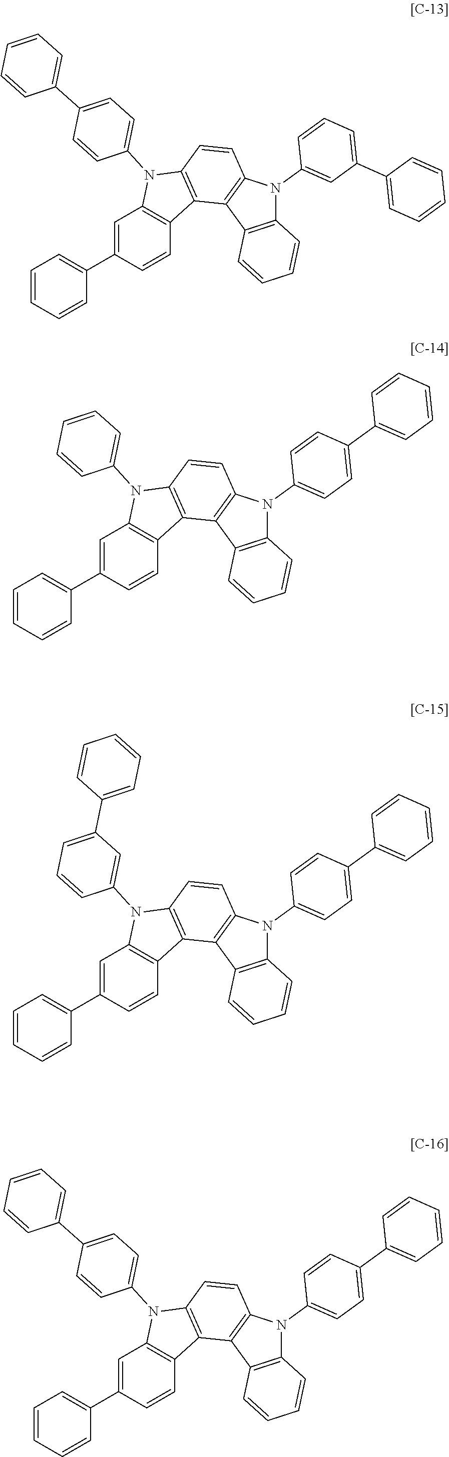 [SMILES: c1ccc(-c2ccc(-n3c4cc(-c5ccccc5)ccc4c4c5c6ccccc6n(-c6cccc(-c7ccccc7)c6)c5ccc43)cc2)cc1 ; c1ccc(-c2ccc(-n3c4ccccc4c4c5c6ccc(-c7ccccc7)cc6n(-c6ccccc6)c5ccc43)cc2)cc1 ; c1ccc(-c2ccc(-n3c4ccccc4c4c5c6ccc(-c7ccccc7)cc6n(-c6cccc(-c7ccccc7)c6)c5ccc43)cc2)cc1 ; c1ccc(-c2ccc(-n3c4ccccc4c4c5c6ccc(-c7ccccc7)cc6n(-c6ccc(-c7ccccc7)cc6)c5ccc43)cc2)cc1]

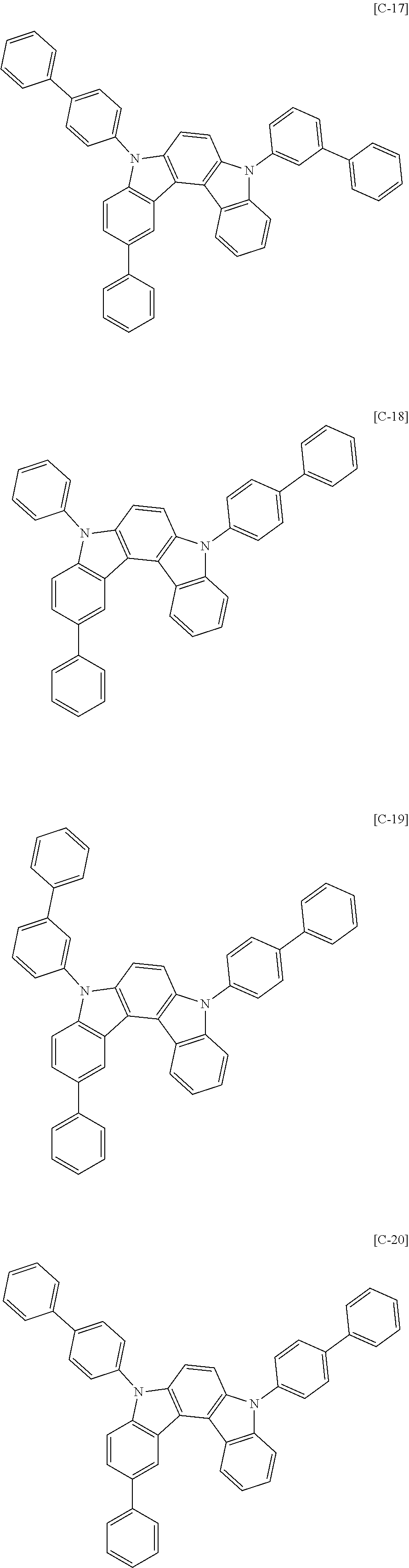 [SMILES: c1ccc(-c2ccc(-n3c4ccc(-c5ccccc5)cc4c4c5c6ccccc6n(-c6cccc(-c7ccccc7)c6)c5ccc43)cc2)cc1 ; c1ccc(-c2ccc(-n3c4ccccc4c4c5c6cc(-c7ccccc7)ccc6n(-c6ccccc6)c5ccc43)cc2)cc1 ; c1ccc(-c2ccc(-n3c4ccccc4c4c5c6cc(-c7ccccc7)ccc6n(-c6cccc(-c7ccccc7)c6)c5ccc43)cc2)cc1 ; c1ccc(-c2ccc(-n3c4ccccc4c4c5c6cc(-c7ccccc7)ccc6n(-c6ccc(-c7ccccc7)cc6)c5ccc43)cc2)cc1]

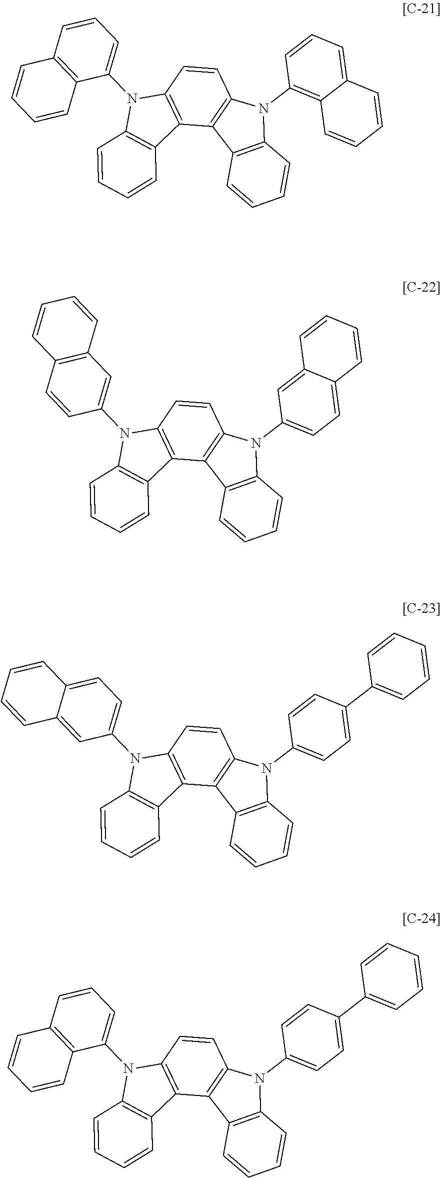 [SMILES: c1ccc2c(-n3c4ccccc4c4c5c6ccccc6n(-c6cccc7ccccc67)c5ccc43)cccc2c1 ; c1ccc2cc(-n3c4ccccc4c4c5c6ccccc6n(-c6ccc7ccccc7c6)c5ccc43)ccc2c1 ; c1ccc(-c2ccc(-n3c4ccccc4c4c5c6ccccc6n(-c6ccc7ccccc7c6)c5ccc43)cc2)cc1 ; c1ccc(-c2ccc(-n3c4ccccc4c4c5c6ccccc6n(-c6cccc7ccccc67)c5ccc43)cc2)cc1]

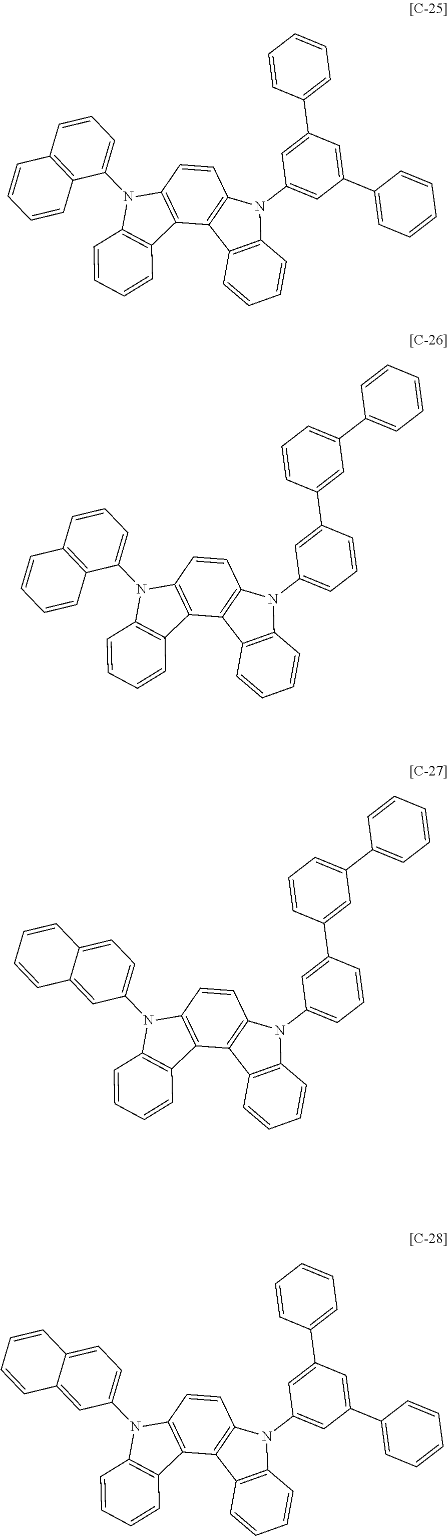 [SMILES: c1ccc(-c2cc(-c3ccccc3)cc(-n3c4ccccc4c4c5c6ccccc6n(-c6cccc7ccccc67)c5ccc43)c2)cc1 ; c1ccc(-c2cccc(-c3cccc(-n4c5ccccc5c5c6c7ccccc7n(-c7cccc8ccccc78)c6ccc54)c3)c2)cc1 ; c1ccc(-c2cccc(-c3cccc(-n4c5ccccc5c5c6c7ccccc7n(-c7ccc8ccccc8c7)c6ccc54)c3)c2)cc1 ; c1ccc(-c2cc(-c3ccccc3)cc(-n3c4ccccc4c4c5c6ccccc6n(-c6ccc7ccccc7c6)c5ccc43)c2)cc1]

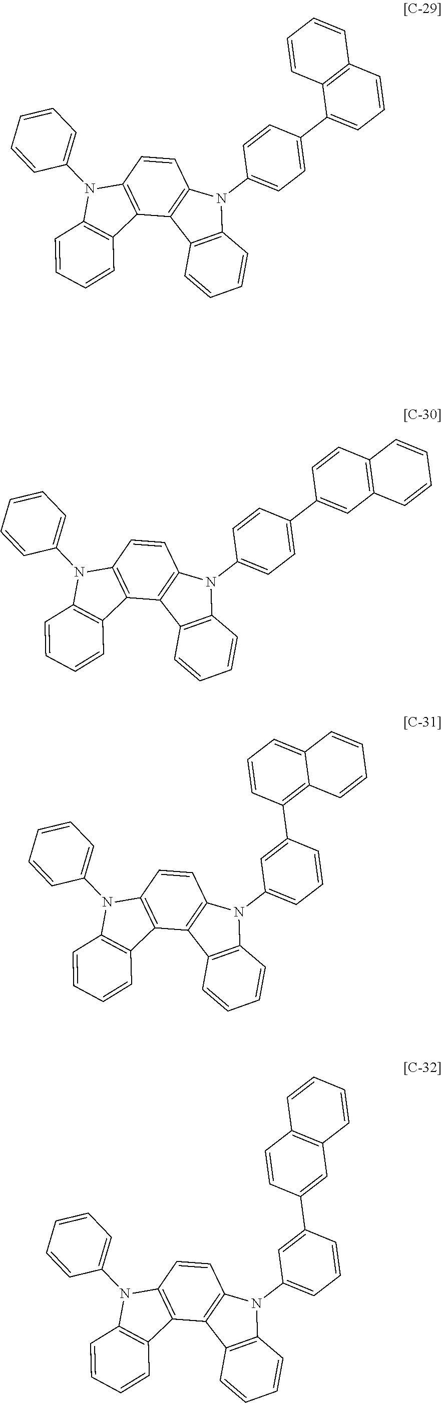 [SMILES: c1ccc(-n2c3ccccc3c3c4c5ccccc5n(-c5ccc(-c6cccc7ccccc67)cc5)c4ccc32)cc1 ; c1ccc(-n2c3ccccc3c3c4c5ccccc5n(-c5ccc(-c6ccc7ccccc7c6)cc5)c4ccc32)cc1 ; c1ccc(-n2c3ccccc3c3c4c5ccccc5n(-c5cccc(-c6cccc7ccccc67)c5)c4ccc32)cc1 ; c1ccc(-n2c3ccccc3c3c4c5ccccc5n(-c5cccc(-c6ccc7ccccc7c6)c5)c4ccc32)cc1]

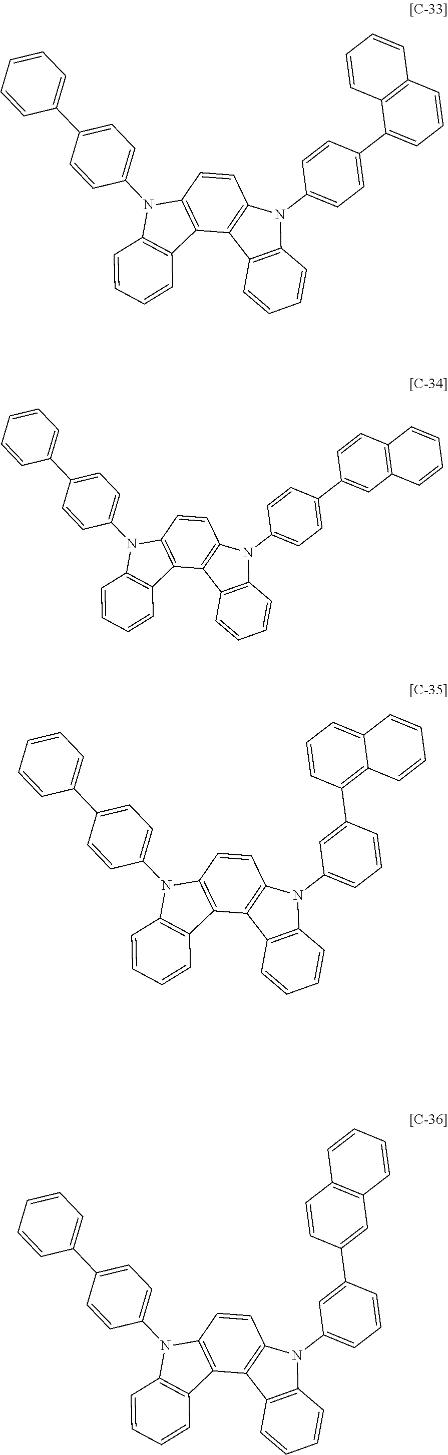 [SMILES: c1ccc(-c2ccc(-n3c4ccccc4c4c5c6ccccc6n(-c6ccc(-c7cccc8ccccc78)cc6)c5ccc43)cc2)cc1 ; c1ccc(-c2ccc(-n3c4ccccc4c4c5c6ccccc6n(-c6ccc(-c7ccc8ccccc8c7)cc6)c5ccc43)cc2)cc1 ; c1ccc(-c2ccc(-n3c4ccccc4c4c5c6ccccc6n(-c6cccc(-c7cccc8ccccc78)c6)c5ccc43)cc2)cc1 ; c1ccc(-c2ccc(-n3c4ccccc4c4c5c6ccccc6n(-c6cccc(-c7ccc8ccccc8c7)c6)c5ccc43)cc2)cc1]

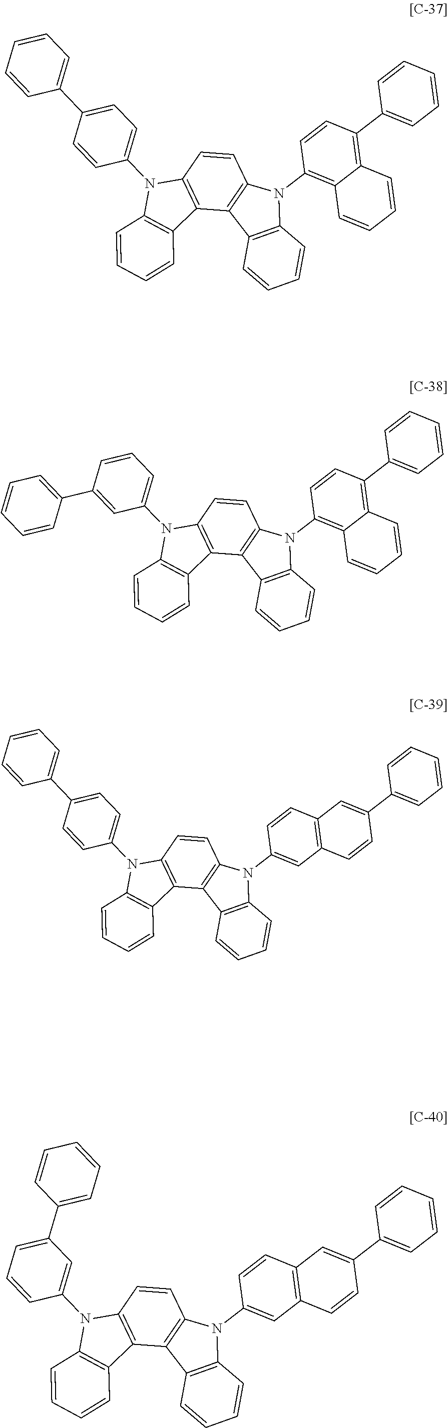 [SMILES: c1ccc(-c2ccc(-n3c4ccccc4c4c5c6ccccc6n(-c6ccc(-c7ccccc7)c7ccccc67)c5ccc43)cc2)cc1 ; c1ccc(-c2cccc(-n3c4ccccc4c4c5c6ccccc6n(-c6ccc(-c7ccccc7)c7ccccc67)c5ccc43)c2)cc1 ; c1ccc(-c2ccc(-n3c4ccccc4c4c5c6ccccc6n(-c6ccc7cc(-c8ccccc8)ccc7c6)c5ccc43)cc2)cc1 ; c1ccc(-c2cccc(-n3c4ccccc4c4c5c6ccccc6n(-c6ccc7cc(-c8ccccc8)ccc7c6)c5ccc43)c2)cc1]

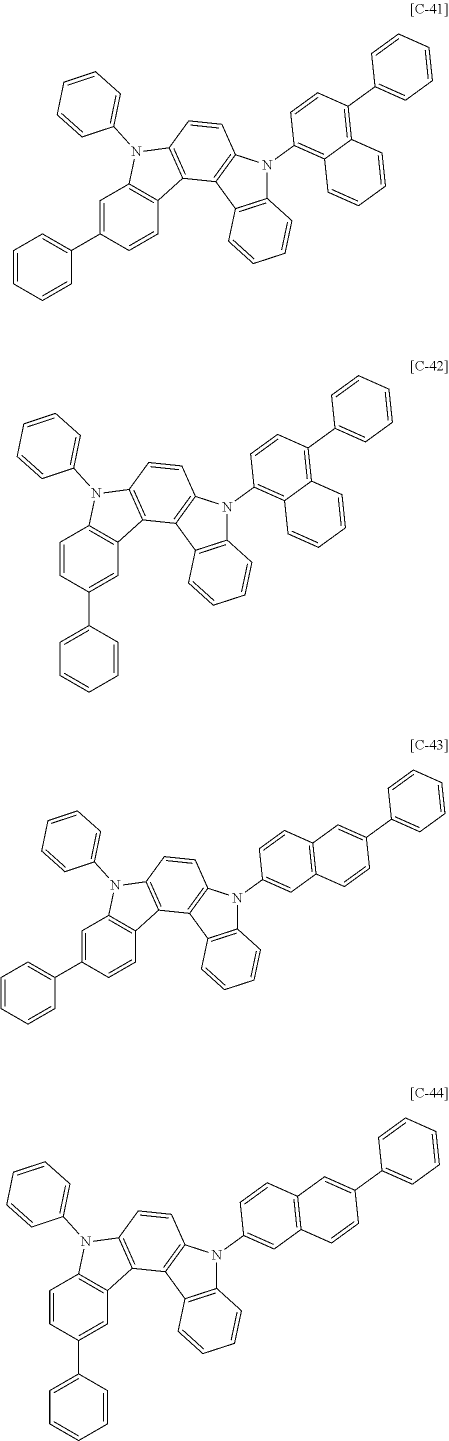 [SMILES: c1ccc(-c2ccc3c4c5c6ccccc6n(-c6ccc(-c7ccccc7)c7ccccc67)c5ccc4n(-c4ccccc4)c3c2)cc1 ; c1ccc(-c2ccc3c(c2)c2c4c5ccccc5n(-c5ccc(-c6ccccc6)c6ccccc56)c4ccc2n3-c2ccccc2)cc1 ; c1ccc(-c2ccc3cc(-n4c5ccccc5c5c6c7ccc(-c8ccccc8)cc7n(-c7ccccc7)c6ccc54)ccc3c2)cc1 ; c1ccc(-c2ccc3cc(-n4c5ccccc5c5c6c7cc(-c8ccccc8)ccc7n(-c7ccccc7)c6ccc54)ccc3c2)cc1]

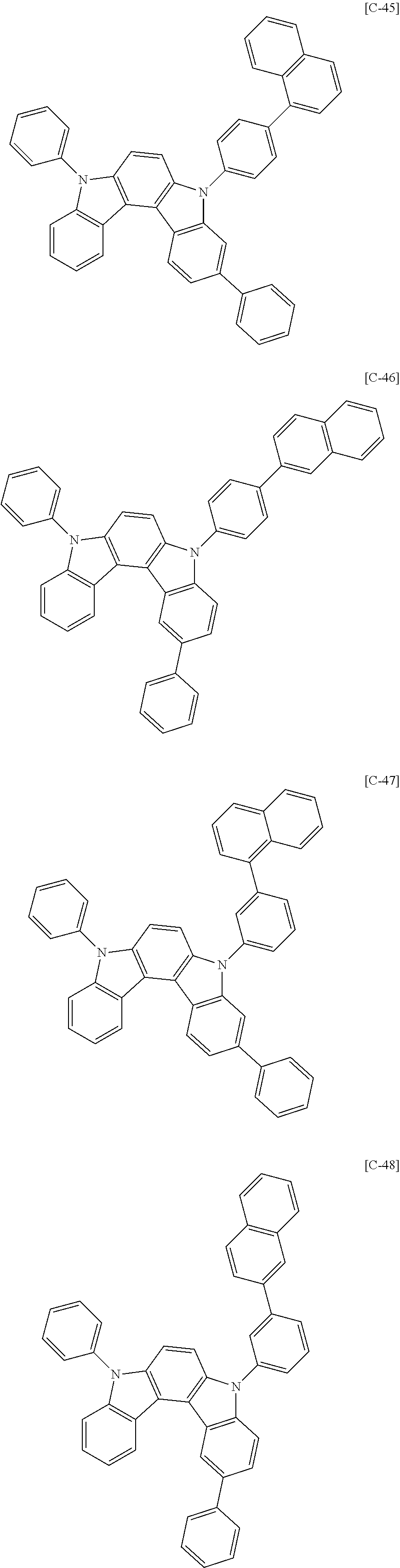 [SMILES: c1ccc(-c2ccc3c4c5c6ccccc6n(-c6ccccc6)c5ccc4n(-c4ccc(-c5cccc6ccccc56)cc4)c3c2)cc1 ; c1ccc(-c2ccc3c(c2)c2c4c5ccccc5n(-c5ccccc5)c4ccc2n3-c2ccc(-c3ccc4ccccc4c3)cc2)cc1 ; c1ccc(-c2ccc3c4c5c6ccccc6n(-c6ccccc6)c5ccc4n(-c4cccc(-c5cccc6ccccc56)c4)c3c2)cc1 ; c1ccc(-c2ccc3c(c2)c2c4c5ccccc5n(-c5ccccc5)c4ccc2n3-c2cccc(-c3ccc4ccccc4c3)c2)cc1]

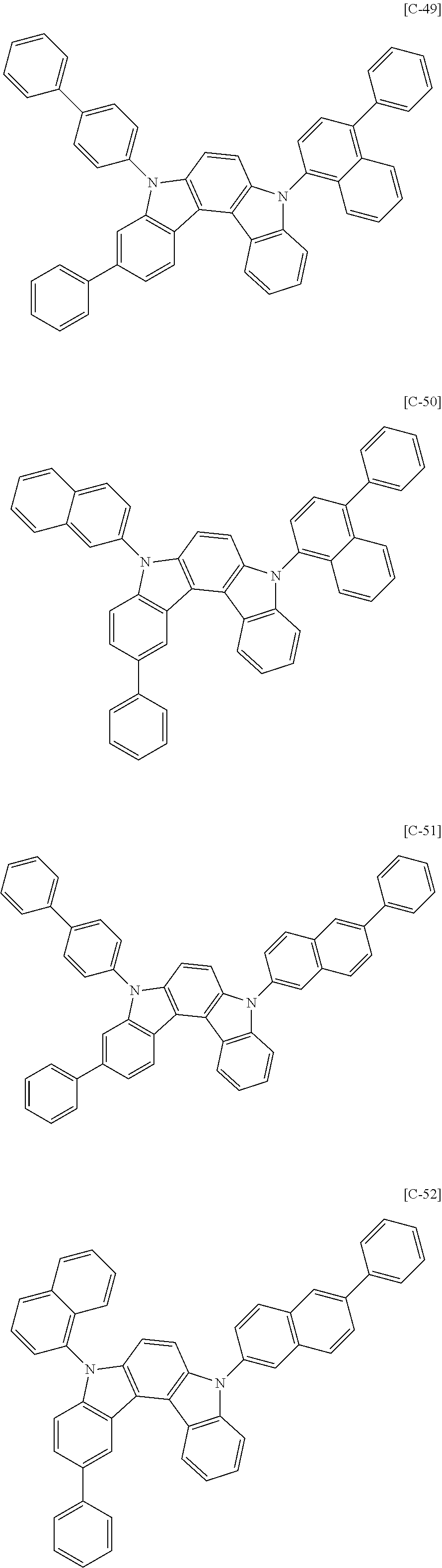 [SMILES: c1ccc(-c2ccc(-n3c4cc(-c5ccccc5)ccc4c4c5c6ccccc6n(-c6ccc(-c7ccccc7)c7ccccc67)c5ccc43)cc2)cc1 ; c1ccc(-c2ccc3c(c2)c2c4c5ccccc5n(-c5ccc(-c6ccccc6)c6ccccc56)c4ccc2n3-c2ccc3ccccc3c2)cc1 ; c1ccc(-c2ccc(-n3c4cc(-c5ccccc5)ccc4c4c5c6ccccc6n(-c6ccc7cc(-c8ccccc8)ccc7c6)c5ccc43)cc2)cc1 ; c1ccc(-c2ccc3cc(-n4c5ccccc5c5c6c7cc(-c8ccccc8)ccc7n(-c7cccc8ccccc78)c6ccc54)ccc3c2)cc1]

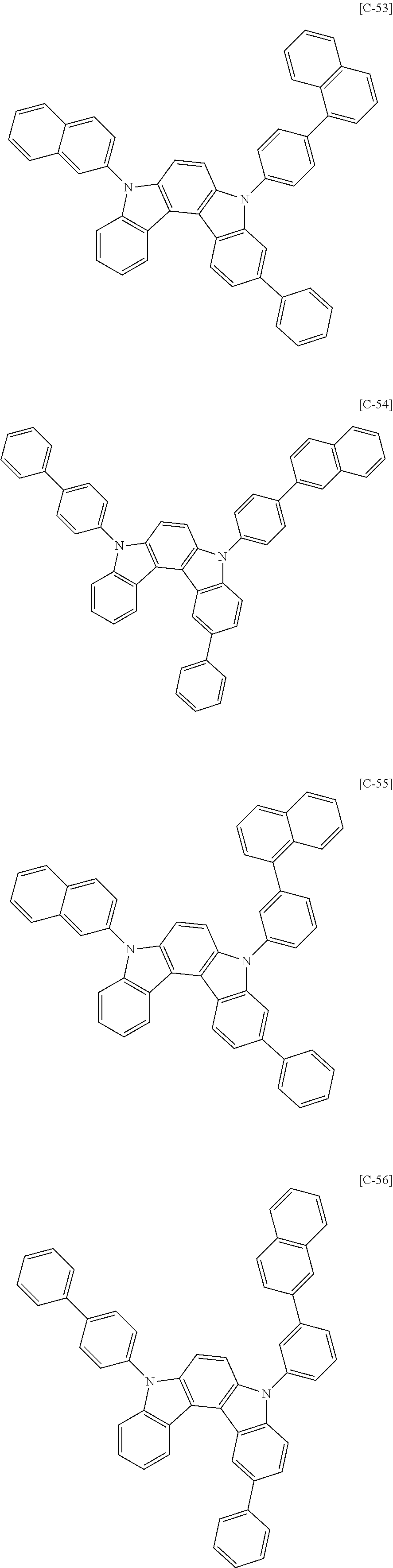 [SMILES: c1ccc(-c2ccc3c4c5c6ccccc6n(-c6ccc7ccccc7c6)c5ccc4n(-c4ccc(-c5cccc6ccccc56)cc4)c3c2)cc1 ; c1ccc(-c2ccc(-n3c4ccccc4c4c5c6cc(-c7ccccc7)ccc6n(-c6ccc(-c7ccc8ccccc8c7)cc6)c5ccc43)cc2)cc1 ; c1ccc(-c2ccc3c4c5c6ccccc6n(-c6ccc7ccccc7c6)c5ccc4n(-c4cccc(-c5cccc6ccccc56)c4)c3c2)cc1 ; c1ccc(-c2ccc(-n3c4ccccc4c4c5c6cc(-c7ccccc7)ccc6n(-c6cccc(-c7ccc8ccccc8c7)c6)c5ccc43)cc2)cc1]

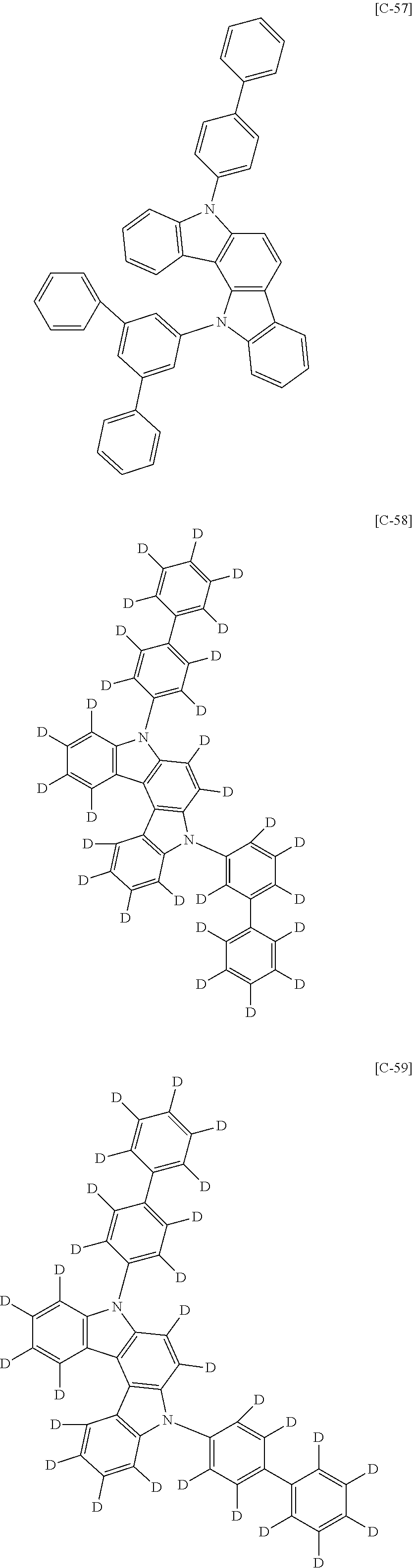

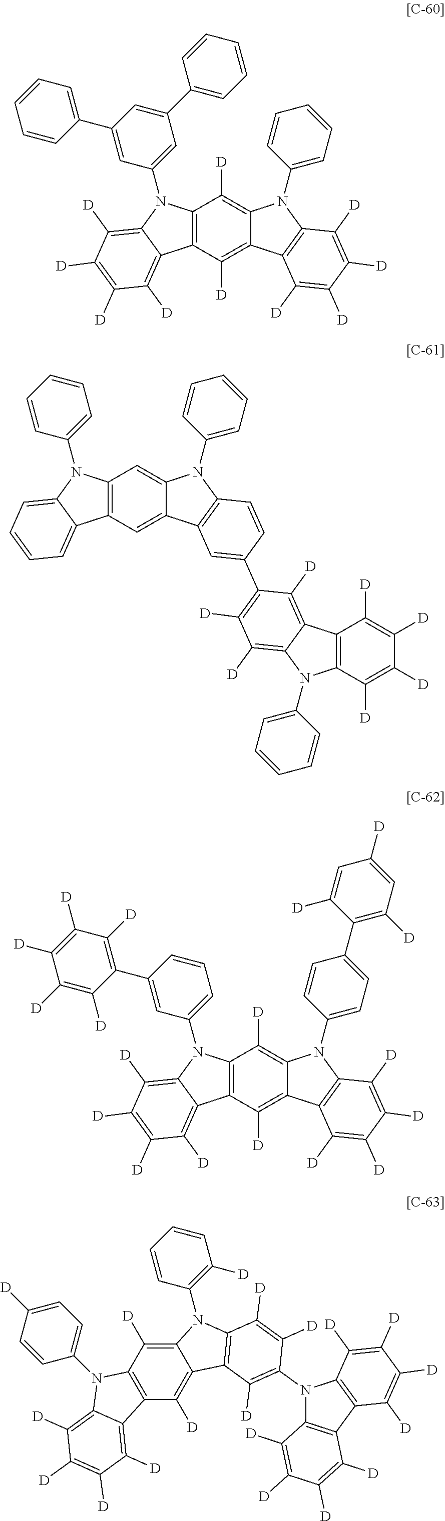

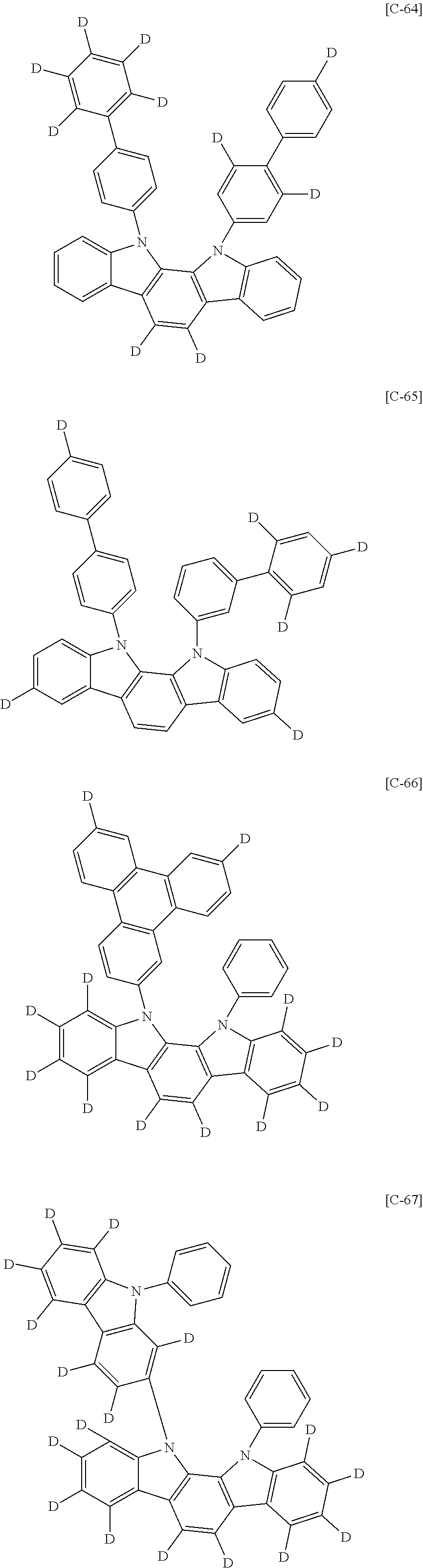

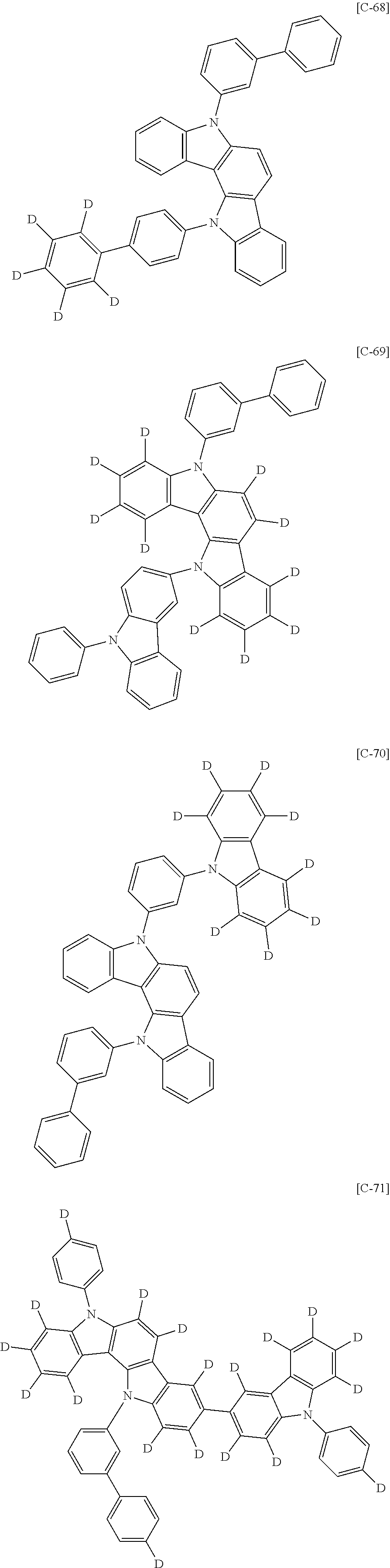

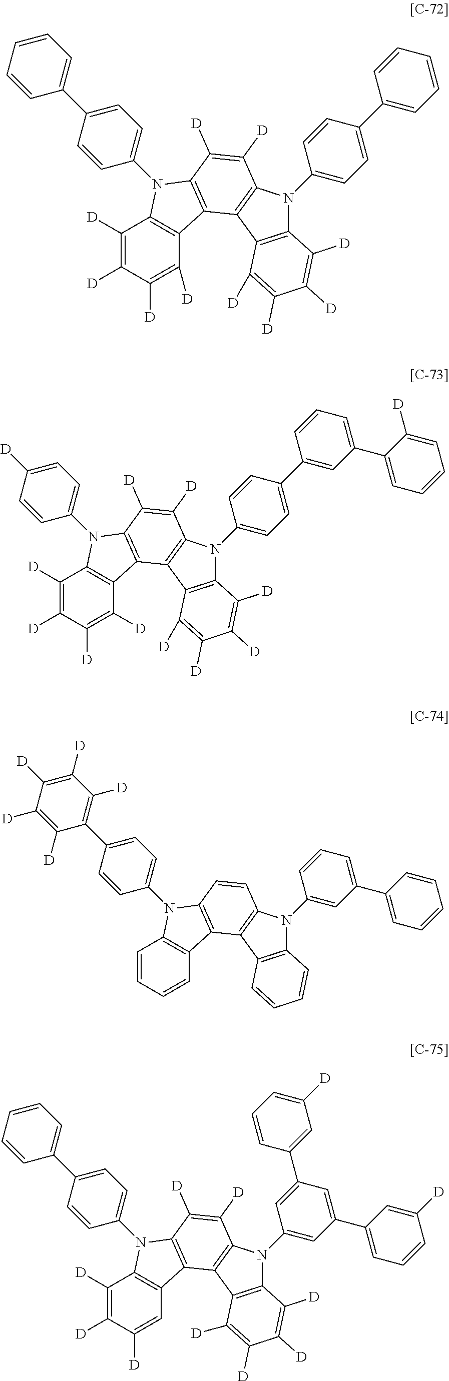

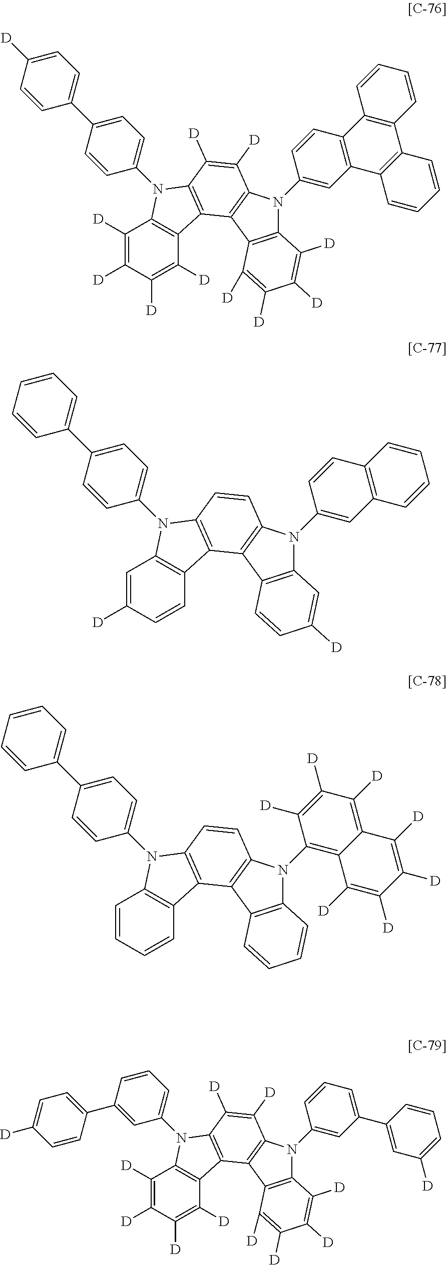 [SMILES: [2H]c1ccc(-c2ccc(-n3c4c([2H])c([2H])c([2H])c([2H])c4c4c5c6c([2H])c([2H])c([2H])c([2H])c6n(-c6ccc7c8ccccc8c8ccccc8c7c6)c5c([2H])c([2H])c43)cc2)cc1 ; [2H]c1ccc2c3c4c5ccc([2H])cc5n(-c5ccc6ccccc6c5)c4ccc3n(-c3ccc(-c4ccccc4)cc3)c2c1 ; [2H]c1c([2H])c([2H])c2c(-n3c4ccccc4c4c5c6ccccc6n(-c6ccc(-c7ccccc7)cc6)c5ccc43)c([2H])c([2H])c([2H])c2c1[2H] ; [2H]c1ccc(-c2cccc(-n3c4c([2H])c([2H])c([2H])c([2H])c4c4c5c6c([2H])c([2H])c([2H])c([2H])c6n(-c6cccc(-c7cccc([2H])c7)c6)c5c([2H])c([2H])c43)c2)cc1]

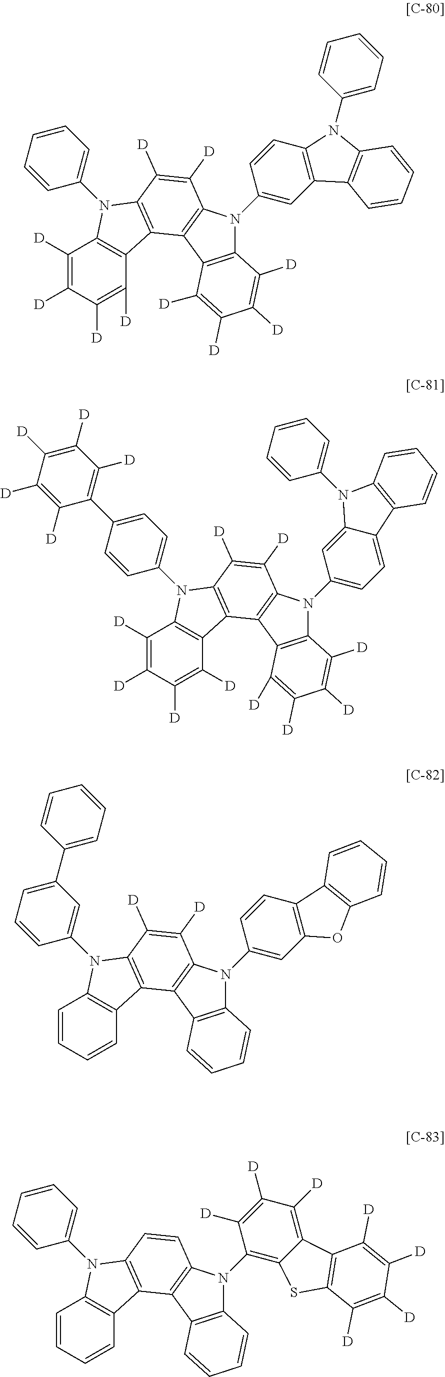 [SMILES: [2H]c1c([2H])c([2H])c2c(c1[2H])c1c3c4c([2H])c([2H])c([2H])c([2H])c4n(-c4ccc5c(c4)c4ccccc4n5-c4ccccc4)c3c([2H])c([2H])c1n2-c1ccccc1 ; [2H]c1c([2H])c([2H])c(-c2ccc(-n3c4c([2H])c([2H])c([2H])c([2H])c4c4c5c6c([2H])c([2H])c([2H])c([2H])c6n(-c6ccc7c8ccccc8n(-c8ccccc8)c7c6)c5c([2H])c([2H])c43)cc2)c([2H])c1[2H] ; [2H]c1c([2H])c2c(c3ccccc3n2-c2ccc3c(c2)oc2ccccc23)c2c3ccccc3n(-c3cccc(-c4ccccc4)c3)c12 ; [2H]c1c([2H])c([2H])c2c(sc3c(-n4c5ccccc5c5c6c7ccccc7n(-c7ccccc7)c6ccc54)c([2H])c([2H])c([2H])c32)c1[2H]]

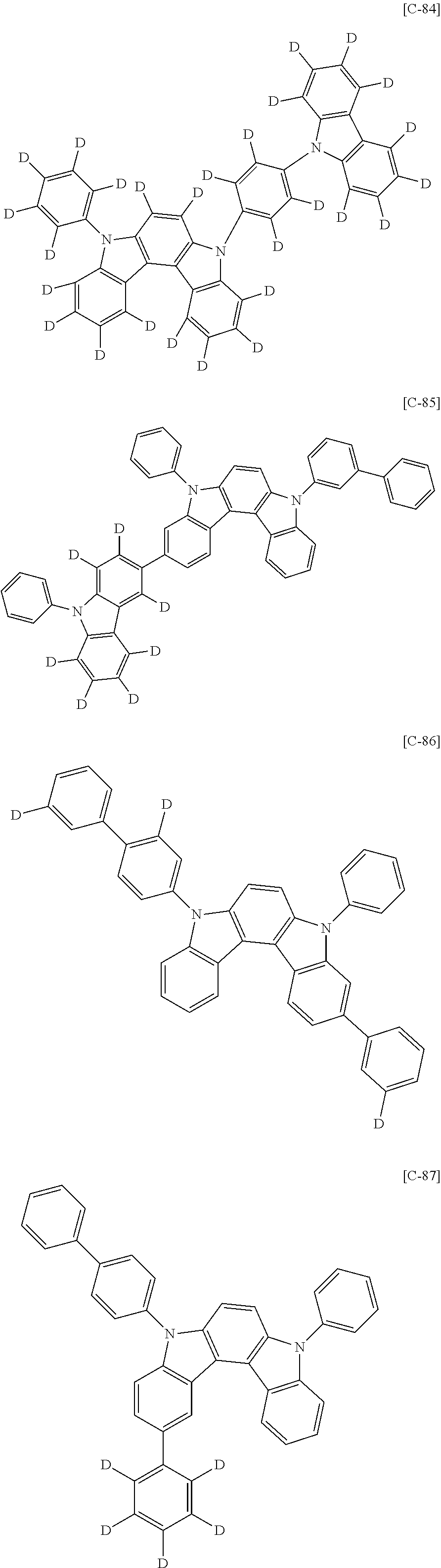

The first compound and the second compound may be included in a weight ratio of, for example, 1:99 to 99:1. By being included in the above range, efficiency and life-span can be improved by implementing bipolar characteristics by adjusting the appropriate weight ratio using the electron transport capability of the first compound and the hole transport capability of the 10 second compound. Within the above range, they may be included in a weight ratio of, for example, about 10:90 to 90:10, about 20:80 to 80:20, for example about 20:80 to about 70:30, about 20:80 to about 60:40, and about 20:80 to about 50:50. As a specific example, they may be included in a weight ratio of 20:80, 30:70, or 40:60.

In addition to the aforementioned first compound and second compound described above, one or more compounds may be further included.

The aforementioned compound for an organic optoelectronic device or a composition for an organic optoelectronic device may further includes a dopant.

The dopant may be, for example, a phosphorescent dopant, such as a red, green, or blue phosphorescent dopant, and may be, for example, a red or green phosphorescent dopant.

The dopant is a material mixed with the compound for an organic optoelectronic device in a small amount to cause light emission and may be generally a material such as a metal complex that emits light by multiple excitation into a triplet or more. The dopant may be, for example an inorganic, organic, or organic-inorganic compound, and one or more types thereof may be used.

Examples of the dopant may be a phosphorescent dopant and examples of the phosphorescent dopant may be an organic metal compound including Ir, Pt, Os, Ti, Zr, Hf, Eu, Tb, Tm, Fe, Co, Ni, Ru, Rh, Pd, or a combination thereof. The phosphorescent dopant may be, for example a compound represented by Chemical Formula Z, but is not limited thereto.

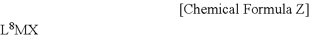

In Chemical Formula Z, M is a metal, and L⁸ and X are the same or different, and are a ligand to form a complex compound with M.

The M may be for example Ir, Pt, Os, Ti, Zr, Hf, Eu, Tb, Tm, Fe, Co, Ni, Ru, Rh, Pd, or a combination thereof, and L⁸ and X may be, for example a bidentate ligand.

Examples of the ligands represented by L⁸ and X may be selected from the Chemical Formulas listed in Group A, but are not limited thereto.

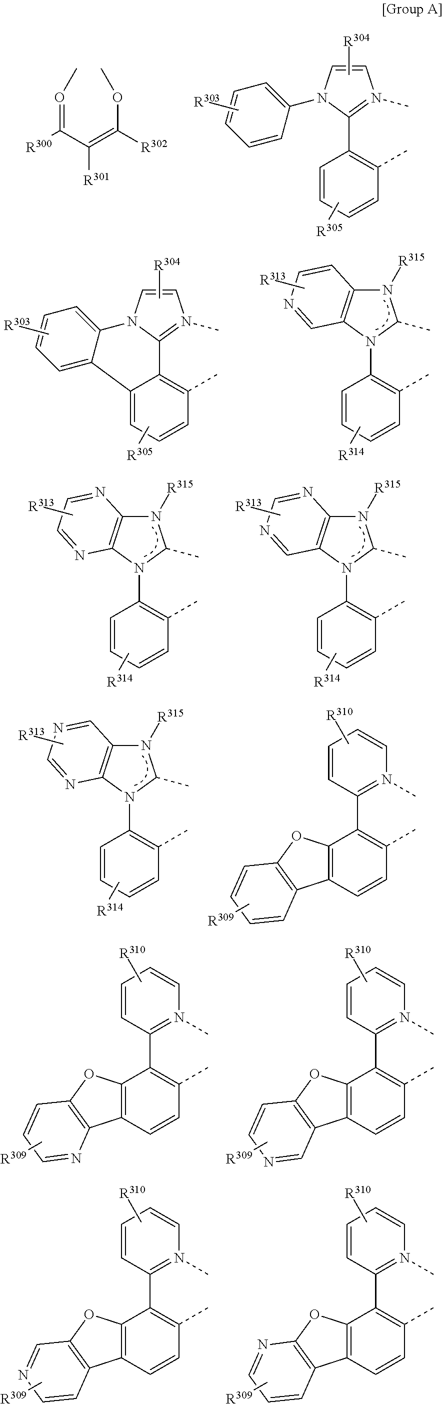

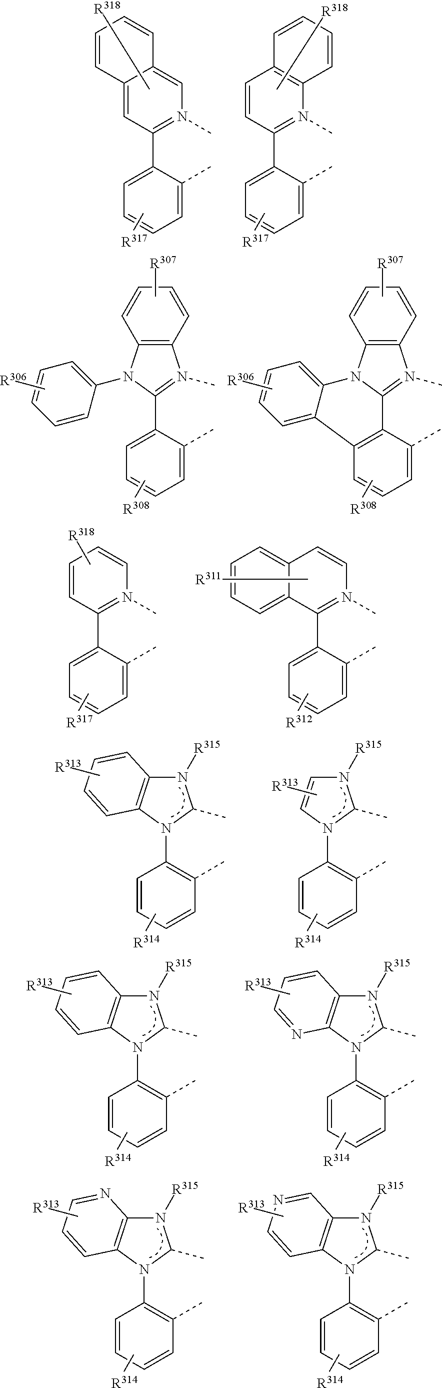

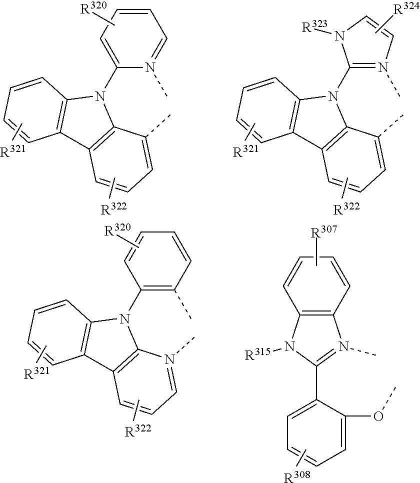

In Group A,
R³⁰⁰ to R³⁰² are each independently hydrogen, deuterium a C1 to C30 alkyl group that is substituted or unsubstituted with a halogen, a C6 to C30 aryl group that is substituted or unsubstituted with a C1 to C30 alkyl, or a halogen, and
R³⁰³ to R³²⁴ are each independently hydrogen, deuterium, a halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SF₅, a trialkylsilyl group having a substituted or unsubstituted C1 to C30 alkyl group, a dialkylarylsilyl group having a substituted or unsubstituted C1 to C30 alkyl group and C6 to C30 aryl group, or a triarylsilyl group having a substituted or unsubstituted C6 to C30 aryl group.

For example, a dopant represented by Chemical Formula V may be included.

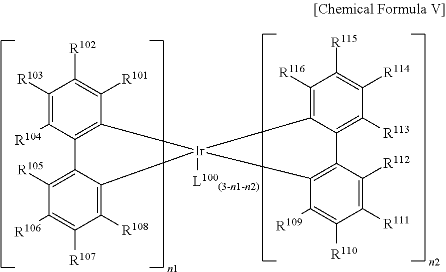

In Chemical Formula V,
R¹⁰¹ to R¹¹⁶ are each independently hydrogen, deuterium, a substituted or unsubstituted C1 to C10 alkyl group, a substituted or unsubstituted C6 to C20 aryl group, or —SiR¹³²R¹³³R¹³⁴,
R¹³² to R¹³⁴ are each independently a C1 to C6 alkyl group,
at least one of R¹⁰¹ to R¹¹⁶ is a functional group represented by Chemical Formula IV-1,
L¹⁰⁰ is a bidentate ligand of a monovalent anion, and is a ligand that coordinates to iridium through a lone pair of carbons or heteroatoms, and
n1 and n2 are each independently any one of integers of 0 to 3, and n1+n2 is any one of integers of 1 to 3,

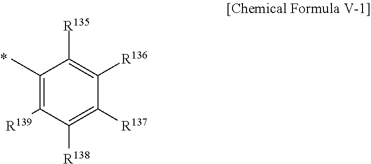

wherein, in Chemical Formula V-1,
R¹³⁵ to R¹³⁹ are each independently hydrogen, deuterium, a substituted or unsubstituted C1 to C10 alkyl group, a substituted or unsubstituted C6 to C20 aryl group, or —SiR¹³²R¹³³R¹³⁴, and
* means a portion linked to a carbon atom.

For example, the dopant represented by Chemical Formula Z-1 may be included.

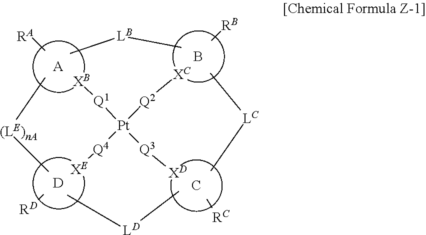

In Chemical Formula Z-1, rings A, B, C, and D are each independently a 5-membered or 6-membered carbocyclic or heterocyclic ring;
R^{A}, R^{B}, R^{C}, and R^{D} are each independently mono-, di-, tri-, or tetra-substitution, or unsubstitution;
L^{B}, L^{C}, and L^{D} are each independently a direct bond, BR, NR, PR, O, S, Se, C═O, S═O, SO₂, CRR′, SiRR′, GeRR′, or a combination thereof,
when nA is 1, L^{E} may be a direct bond, BR, NR, PR, O, S, Se, C═O, S═O, SO₂, CRR′, SiRR′, GeRR′, or a combination thereof, and when nA is 0, L^{E} does not exist;
R^{A}, R^{B}, R^{C}, R^{D}, R, and R′ are each independently hydrogen, deuterium, a halogen, alkyl group, a cycloalkyl group, a heteroalkyl group, arylalkyl group, an alkoxy group, aryloxy group, an amino group, a silyl group, an alkenyl group, a cycloalkenyl group, a heteroalkenyl group, an alkynyl group, aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, or a combination thereof, any adjacent R^{A}, R^{B}, R^{C}, R^{D}, R, and R′ are optionally linked to each other to provide a ring; X^{B}, X^{C}, X^{D}, and X^{E} are each independently selected from carbon and nitrogen; and Q¹, Q², Q³, and Q⁴ each represent oxygen or a direct bond.

The dopant according to an embodiment may be a platinum complex, and may be represented by Chemical Formula VI.

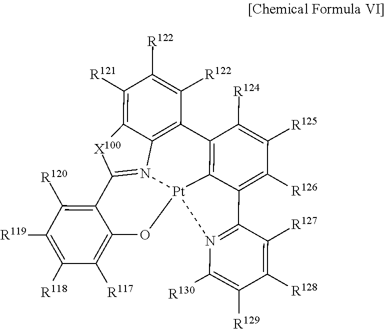

In Chemical Formula VI,
X¹⁰⁰ is selected from O, S, and NR¹³¹,
R¹¹⁷ to R¹³¹ are each independently hydrogen, deuterium, a substituted or unsubstituted C1 to C10 alkyl group, a substituted or unsubstituted C6 to C20 aryl group, or —SiR²¹³³R¹³⁴,
R¹³² to R¹³⁴ are each independently a C1 to C6 alkyl group, and
at least one of R¹¹⁷ to R¹³¹ may be —SiR¹³²R¹³³R¹³⁴ or a tert-butyl group.

Hereinafter, an organic optoelectronic device including the aforementioned compound for an organic optoelectronic device or composition for an organic optoelectronic device will be described.

The organic optoelectronic device may be a suitable device to convert electrical energy into photoenergy and vice versa, e.g., an organic photoelectric device, an organic light emitting diode, an organic solar cell, or an organic photoconductor drum.

Herein, an organic light emitting diode as one example of an organic optoelectronic device is described referring to drawings.

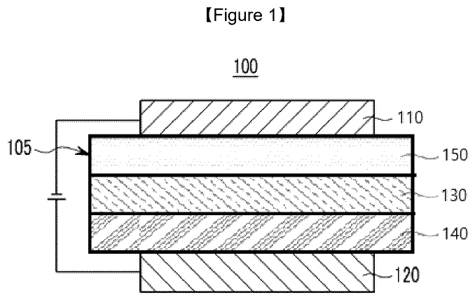

FIG. 1 is a cross-sectional view showing an organic light emitting diode according to an embodiment.

Referring to FIG. 1, an organic light emitting diode 100 according to an embodiment includes an anode 120 and a cathode 110 facing each other and an organic layer 105 disposed between the anode 120 and cathode 110.

The anode 120 may be made of a conductor having a large work function to help hole injection, and may be for example a metal, a metal oxide and/or a conductive polymer. The anode 120 may be, for example a metal such as nickel, platinum, vanadium, chromium, copper, zinc, gold, and the like or an alloy thereof, a metal oxide such as zinc oxide, indium oxide, indium tin oxide (ITO), indium zinc oxide (IZO), and the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, and polyaniline, but is not limited thereto.

The cathode 110 may be made of a conductor having a small work function to help electron injection, and may be for example a metal, a metal oxide, and/or a conductive polymer. The cathode 110 may be for example a metal such as magnesium, calcium, sodium, potassium, titanium, indium, yttrium, lithium, gadolinium, aluminum silver, tin, lead, cesium, barium, and the like, or an alloy thereof; a multi-layer structure material such as LiF/Al, LiO₂/Al, LiF/Ca, and BaF₂/Ca, but is not limited thereto.

The organic layer 105 may include the aforementioned compound for an organic optoelectronic device or composition for an organic optoelectronic device.

The organic layer 105 may include a light emitting layer 130 and the light emitting layer 130 may include the aforementioned compound for an organic optoelectronic device or composition for an organic optoelectronic device.

The composition for an organic optoelectronic device further including a dopant may be, for example, a red or green light emitting composition.

The light emitting layer 130 may include, for example, the aforementioned compound for organic optoelectronic devices or composition for organic optoelectronic devices, as a phosphorescent host.

The organic layer may further include a charge transport region in addition to the light emitting layer.

The charge transport region may be, for example, the hole transport region 140.

The hole transport region 140 may further increase hole injection and/or hole mobility between the anode 120 and the light emitting layer 130 and block electrons.

Specifically, the hole transport region 140 may include a hole transport layer between the anode 120 and the light emitting layer 130, and a hole transport auxiliary layer between the light emitting layer 130 and the hole transport layer, and at least one of the compounds of Group B may be included in at least one of the hole transport layer and the hole transport auxiliary layer.

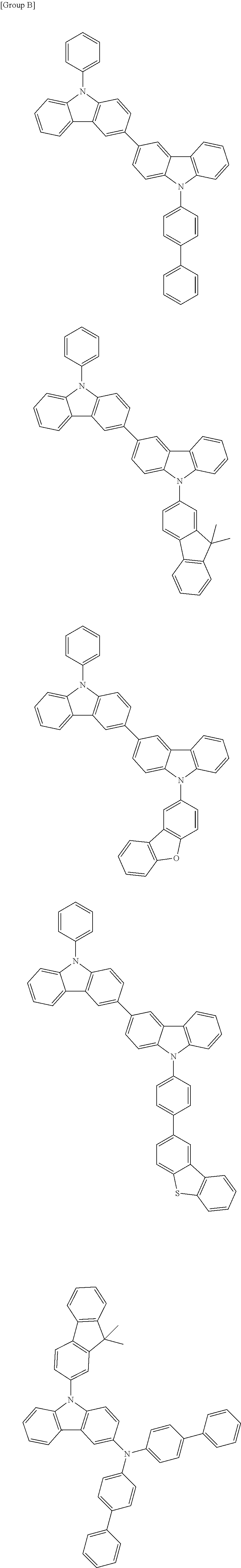 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1 ; c1ccc(-n2c3ccccc3c3cc(-c4ccc5c(c4)c4ccccc4n5-c4ccc(-c5ccc6sc7ccccc7c6c5)cc4)ccc32)cc1 ; CC1(C)c2ccccc2-c2ccc(-n3c4ccccc4c4cc(N(c5ccc(-c6ccccc6)cc5)c5ccc(-c6ccccc6)cc5)ccc43)cc21]

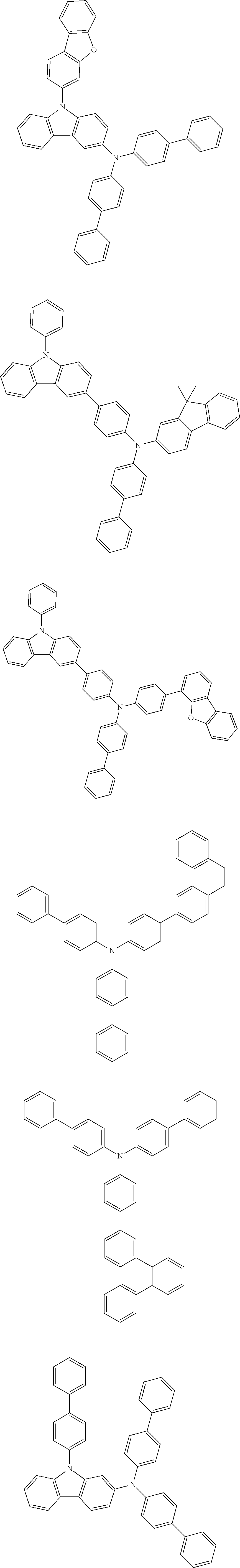

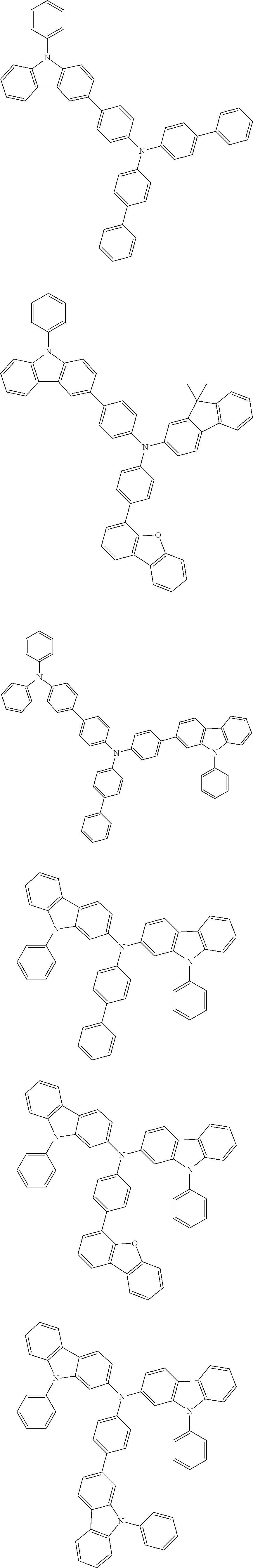

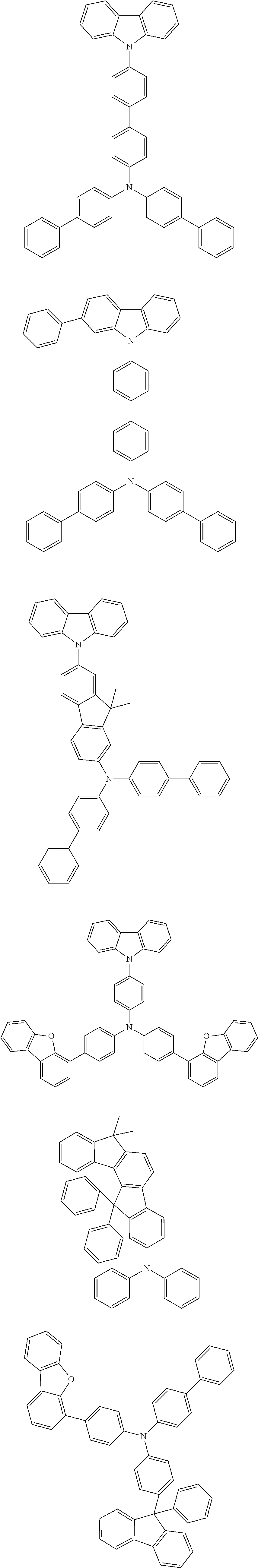

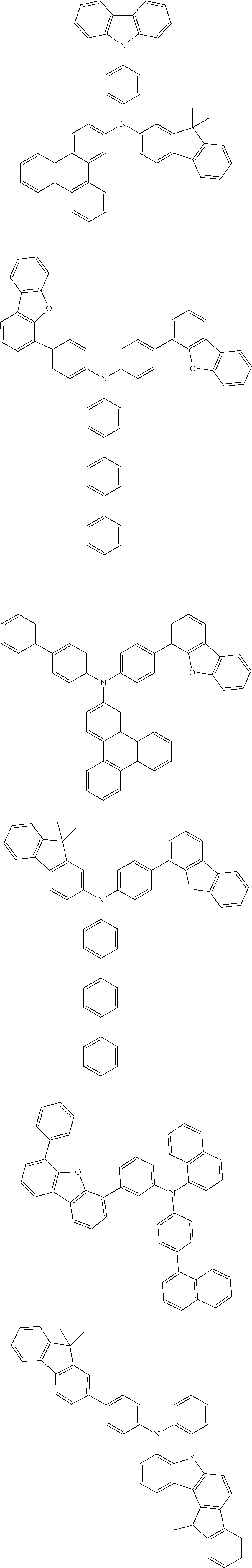

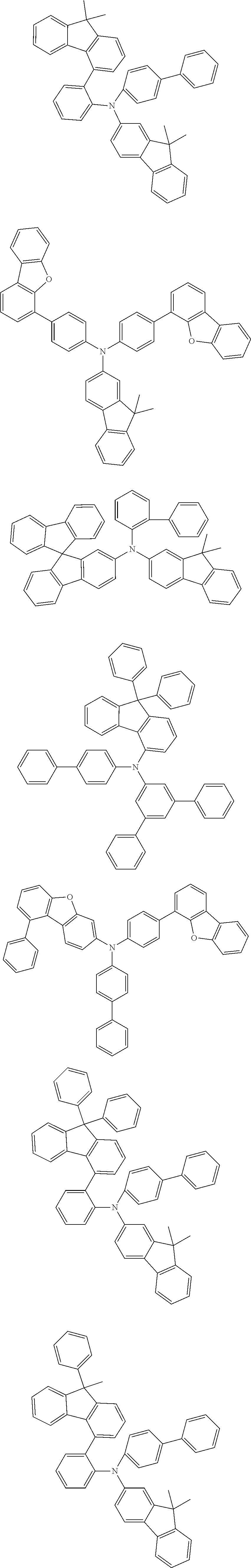

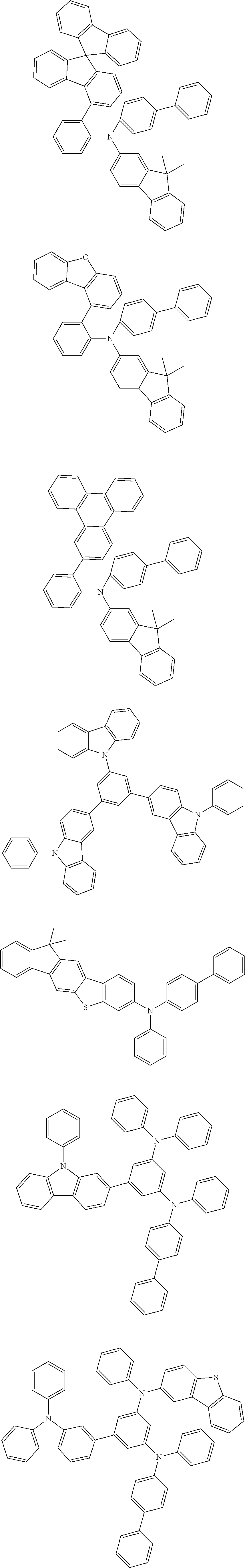

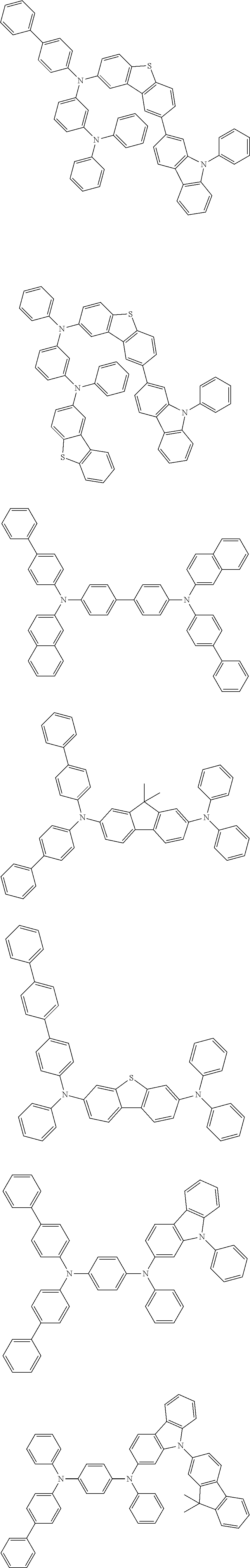

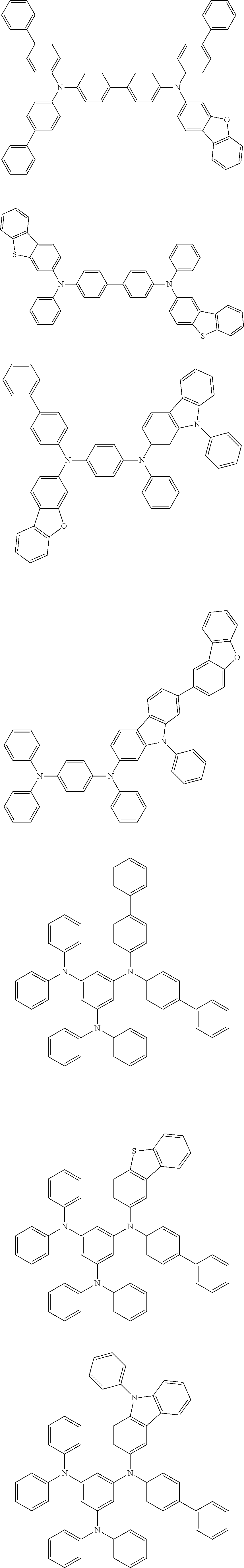

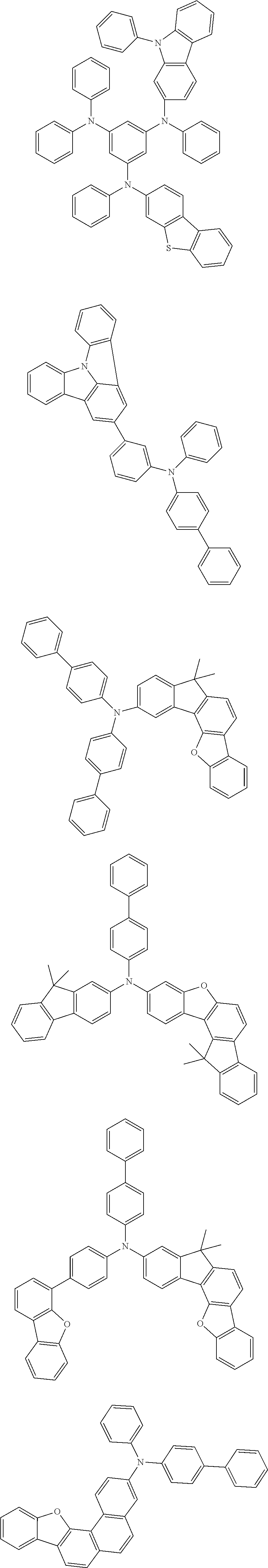

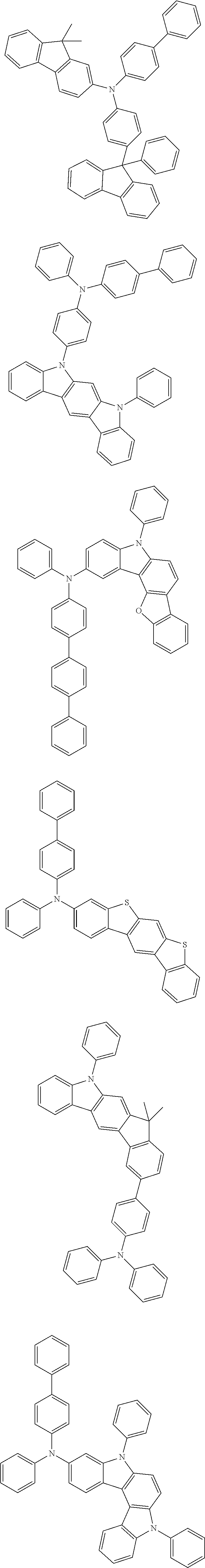 [SMILES: CC1(C)c2ccccc2-c2ccc(N(c3ccc(-c4ccccc4)cc3)c3ccc(C4(c5ccccc5)c5ccccc5-c5ccccc54)cc3)cc21 ; c1ccc(-c2ccc(N(c3ccccc3)c3ccc(-n4c5ccccc5c5cc6c7ccccc7n(-c7ccccc7)c6cc54)cc3)cc2)cc1 ; c1ccc(-c2ccc(-c3ccc(N(c4ccccc4)c4ccc5c(c4)c4c6oc7ccccc7c6ccc4n5-c4ccccc4)cc3)cc2)cc1 ; c1ccc(-c2ccc(N(c3ccccc3)c3ccc4c(c3)sc3cc5sc6ccccc6c5cc34)cc2)cc1 ; CC1(C)c2ccc(-c3ccc(N(c4ccccc4)c4ccccc4)cc3)cc2-c2cc3c4ccccc4n(-c4ccccc4)c3cc21 ; c1ccc(-c2ccc(N(c3ccccc3)c3ccc4c5c6c7ccccc7n(-c7ccccc7)c6ccc5n(-c5ccccc5)c4c3)cc2)cc1]

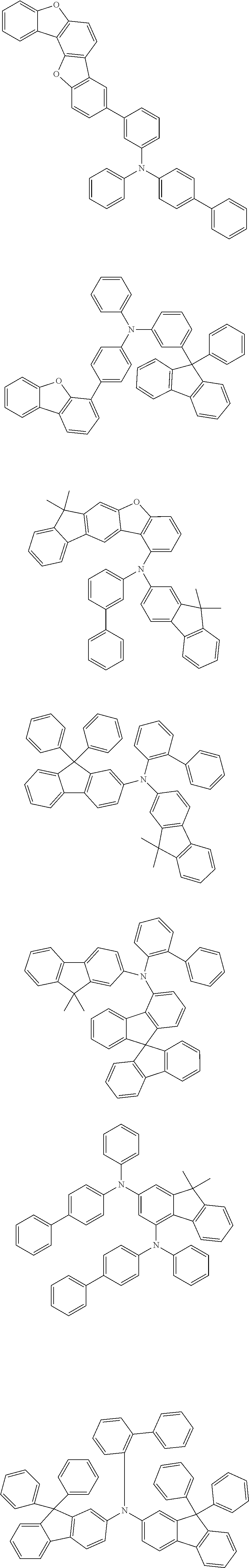

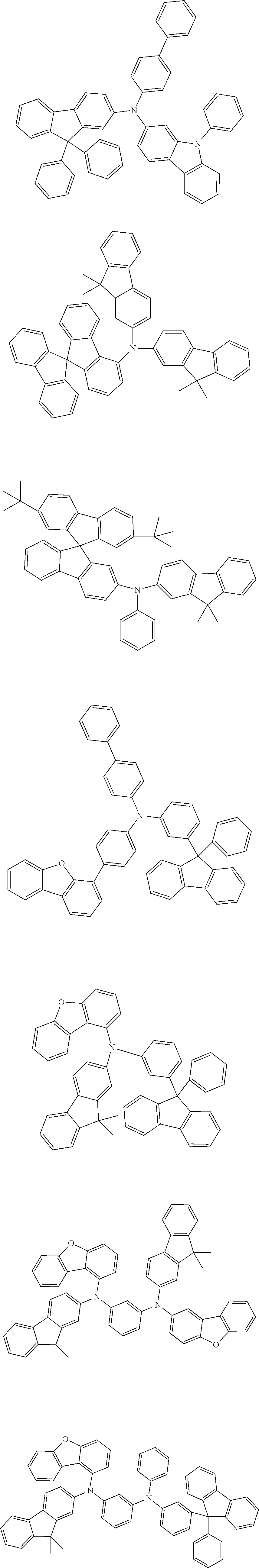

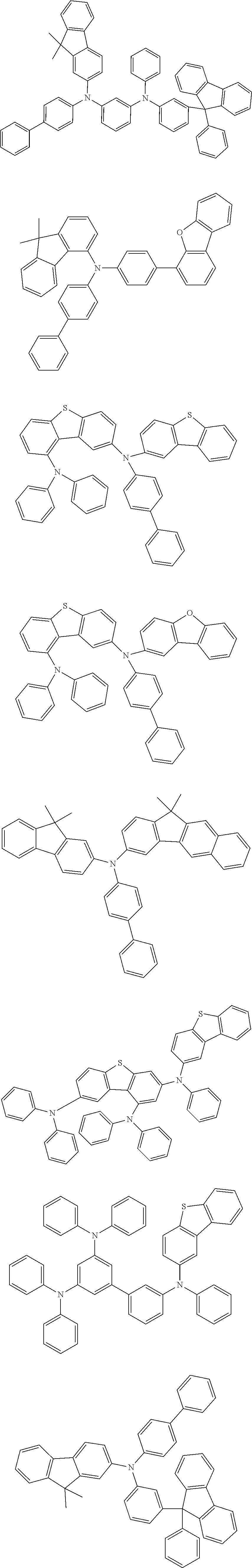

In the hole transport region, in addition to the compounds described above, known compounds disclosed in U.S. Pat. No. 5,061,569A, JP1993-009471A, WO1995-009147A1, JP1995-126615A, JP1998-095973A, etc. and compounds having a similar structure may also be used.

Also, the charge transport region may be, for example, the electron transport region 150.

The electron transport region 150 may further increase electron injection and/or electron mobility and block holes between the cathode 110 and the light emitting layer 130.

Specifically, the electron transport region 150 may include an electron transport layer between the cathode 110 and the light emitting layer 130, and an electron transport auxiliary layer between the light emitting layer 130 and the electron transport layer, and at least one of the compounds of Group C may be included in at least one of the electron transport layer and the electron transport auxiliary layer.

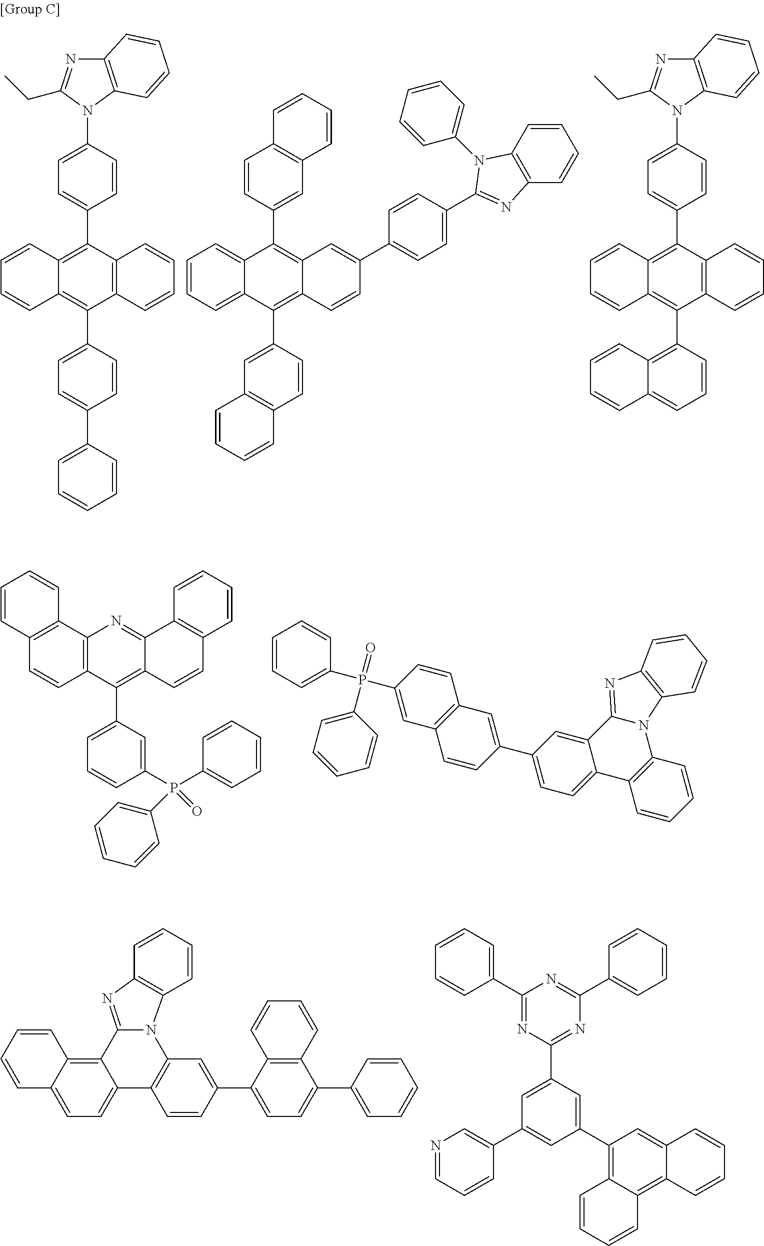

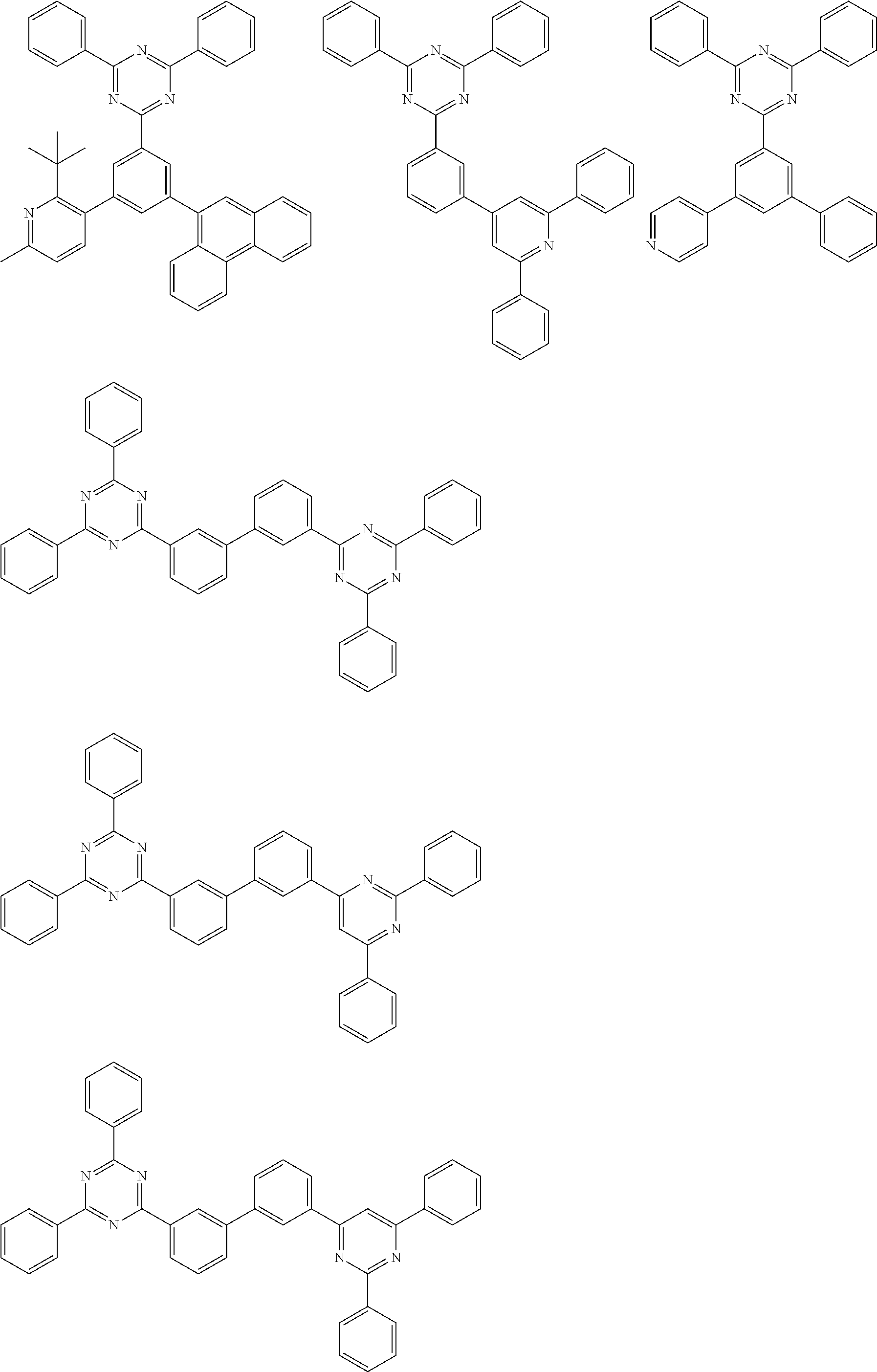

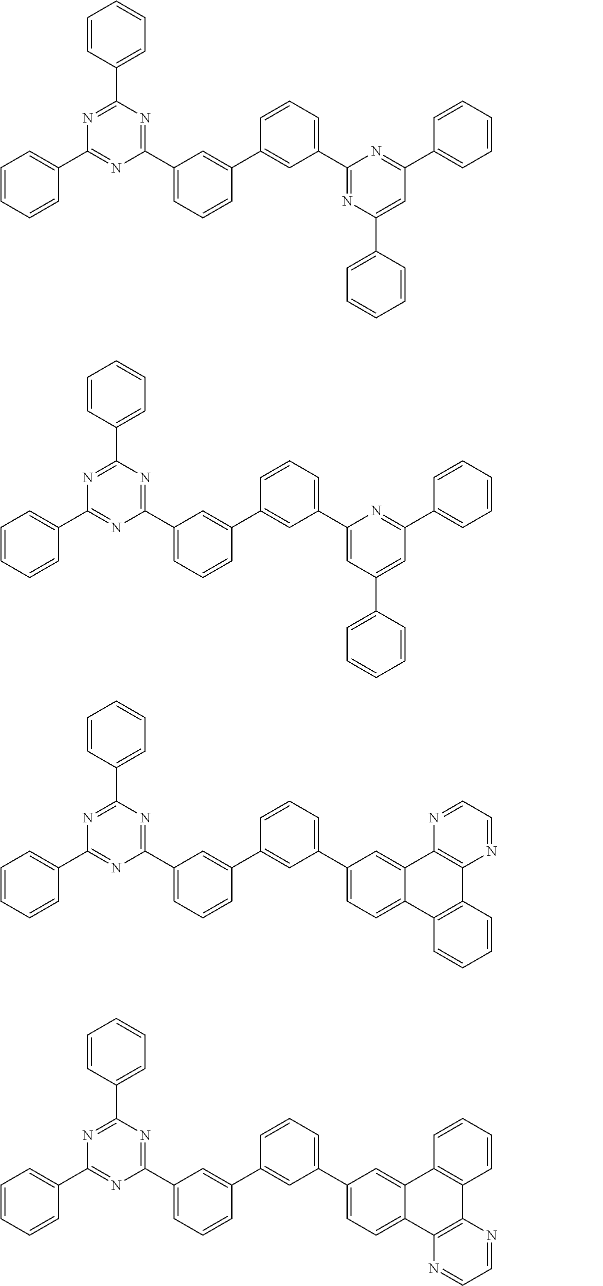 [SMILES: c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1]

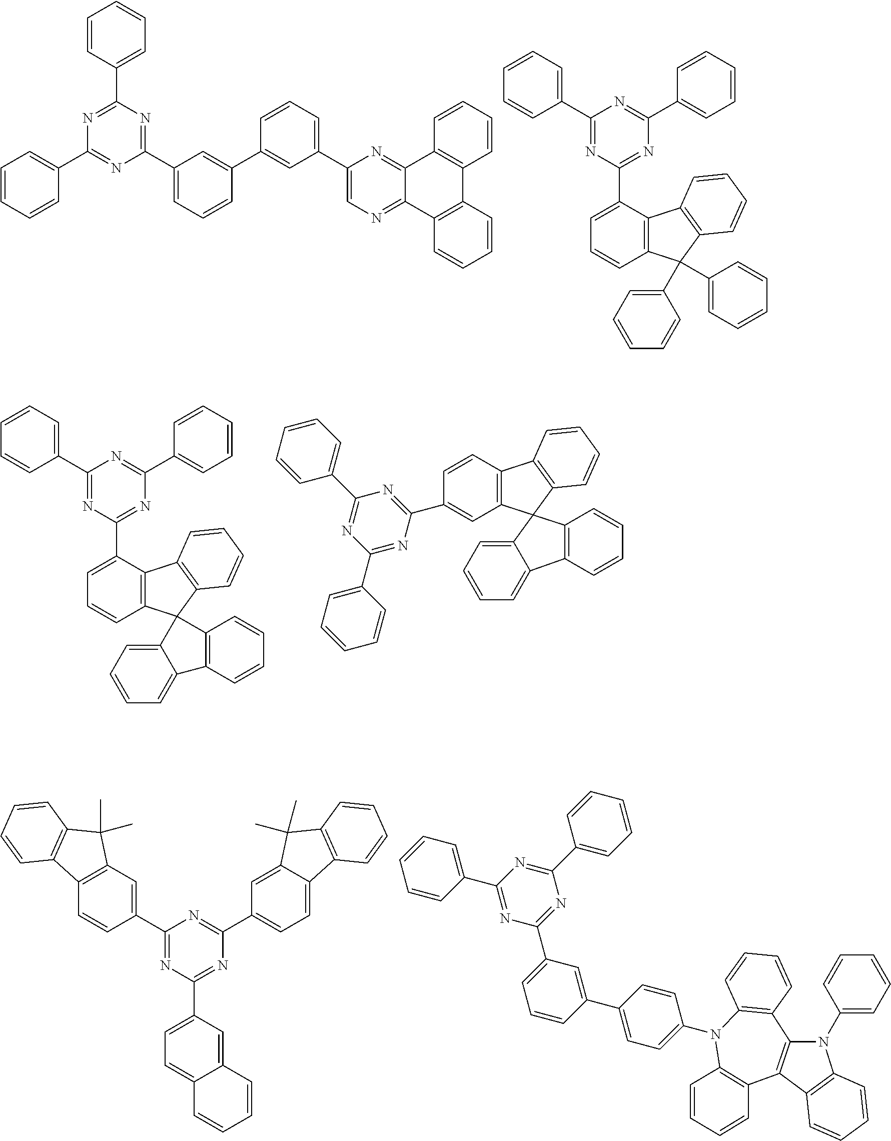

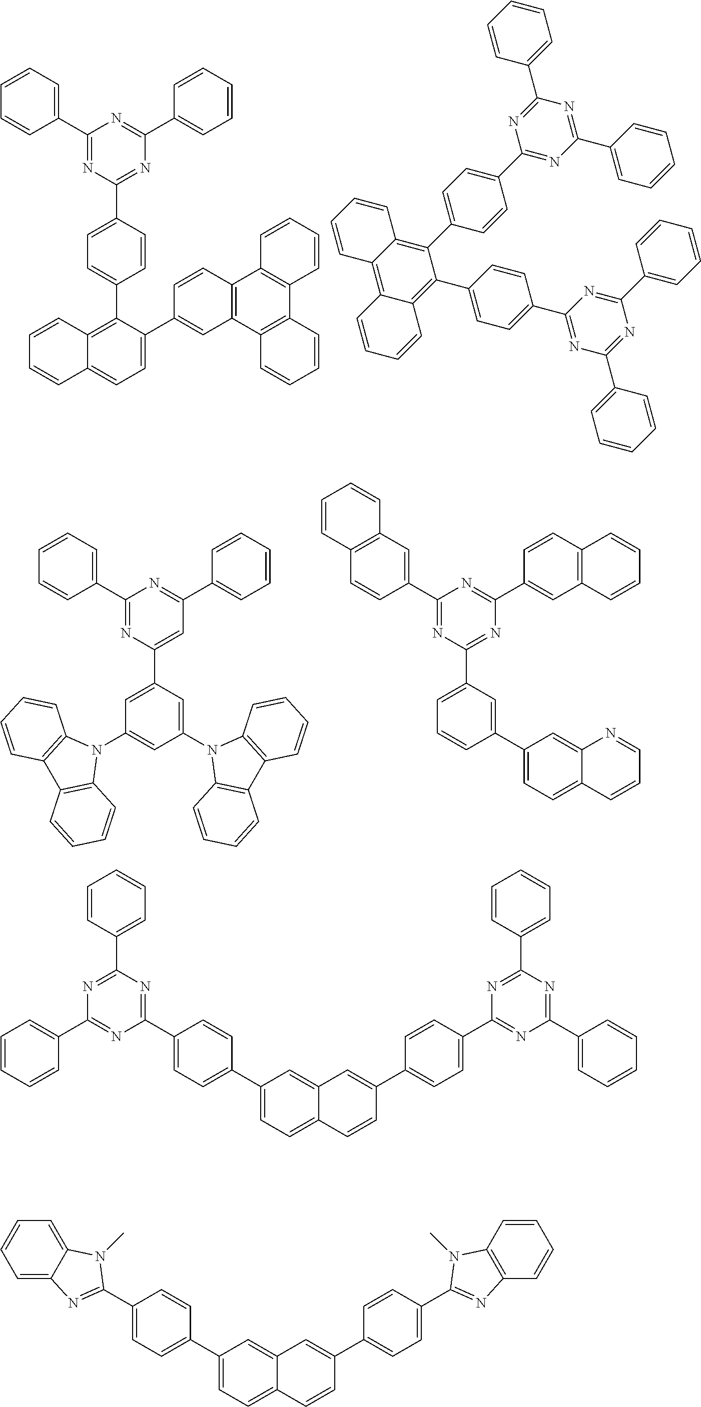

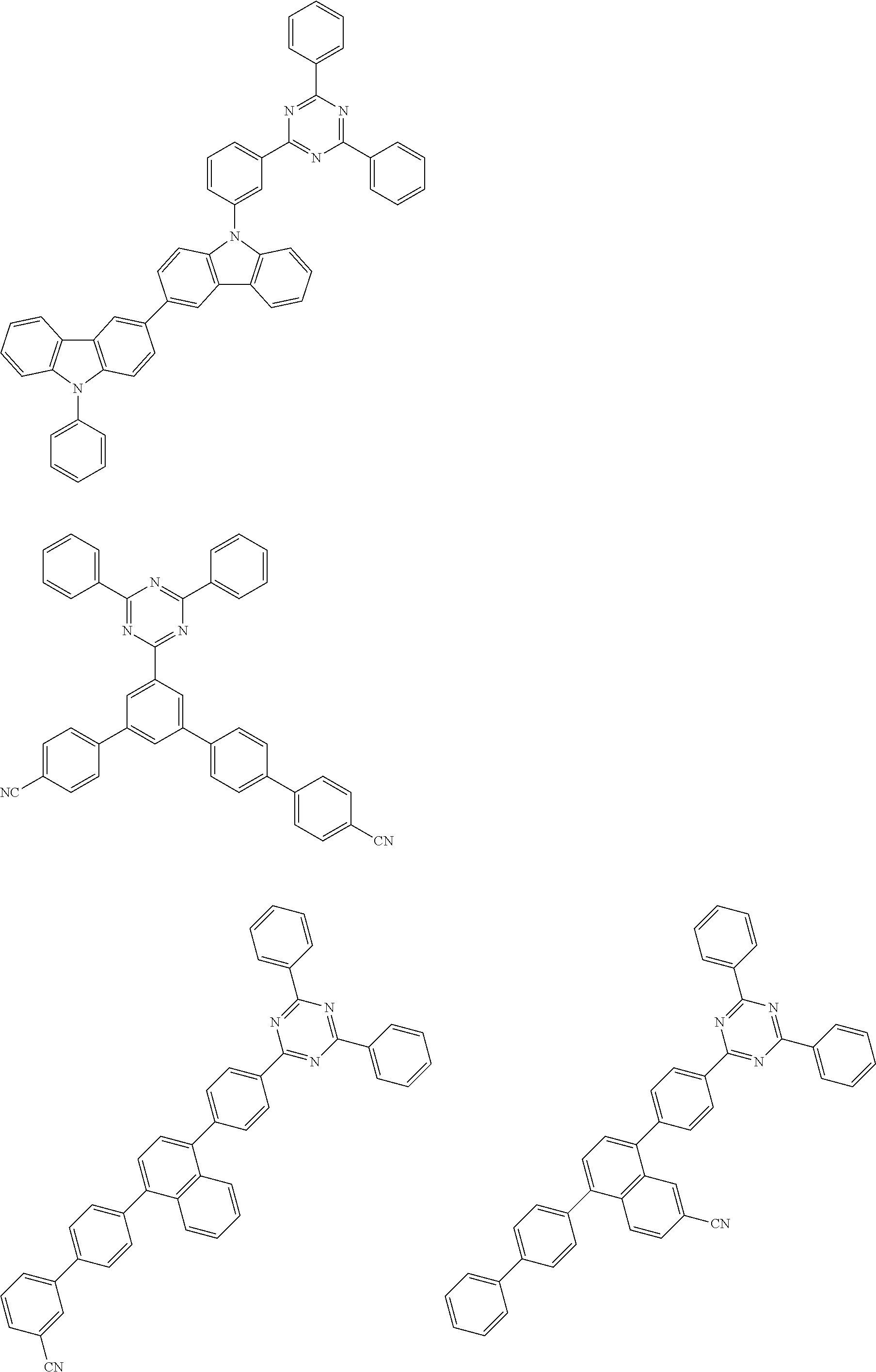 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; N#Cc1ccc(-c2ccc(-c3cc(-c4ccc(C#N)cc4)cc(-c4nc(-c5ccccc5)nc(-c5ccccc5)n4)c3)cc2)cc1 ; N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1]

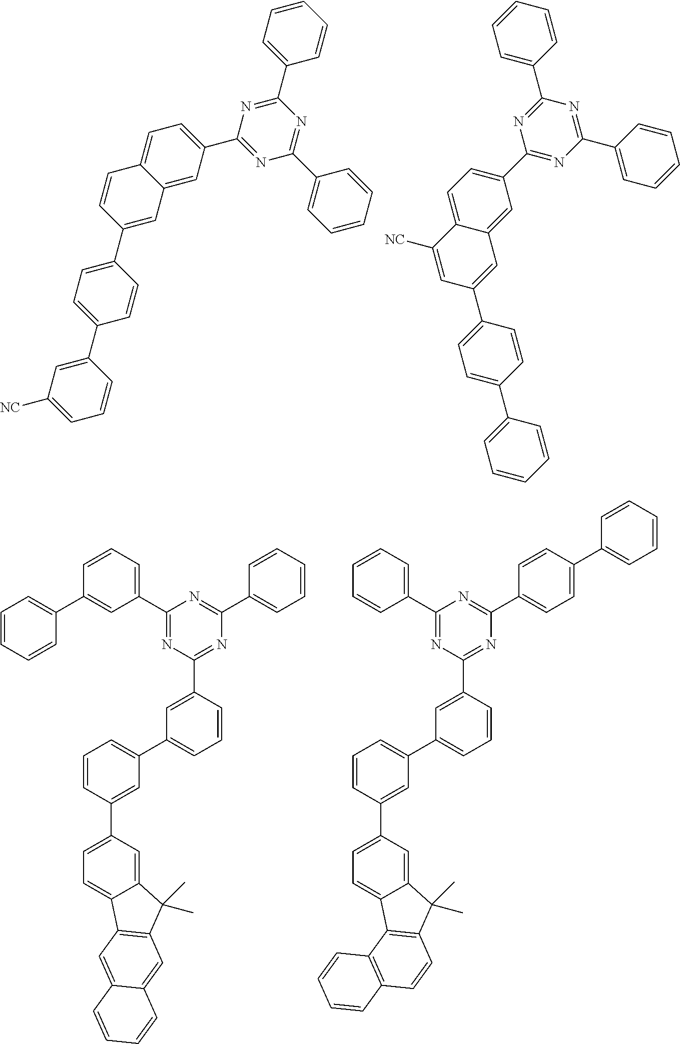

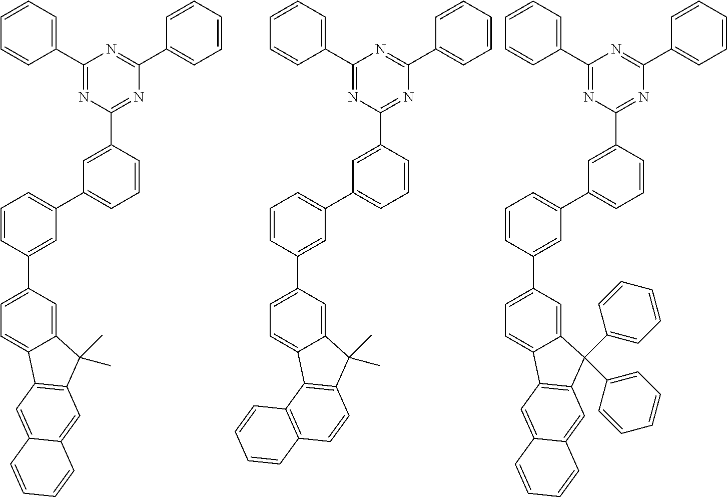

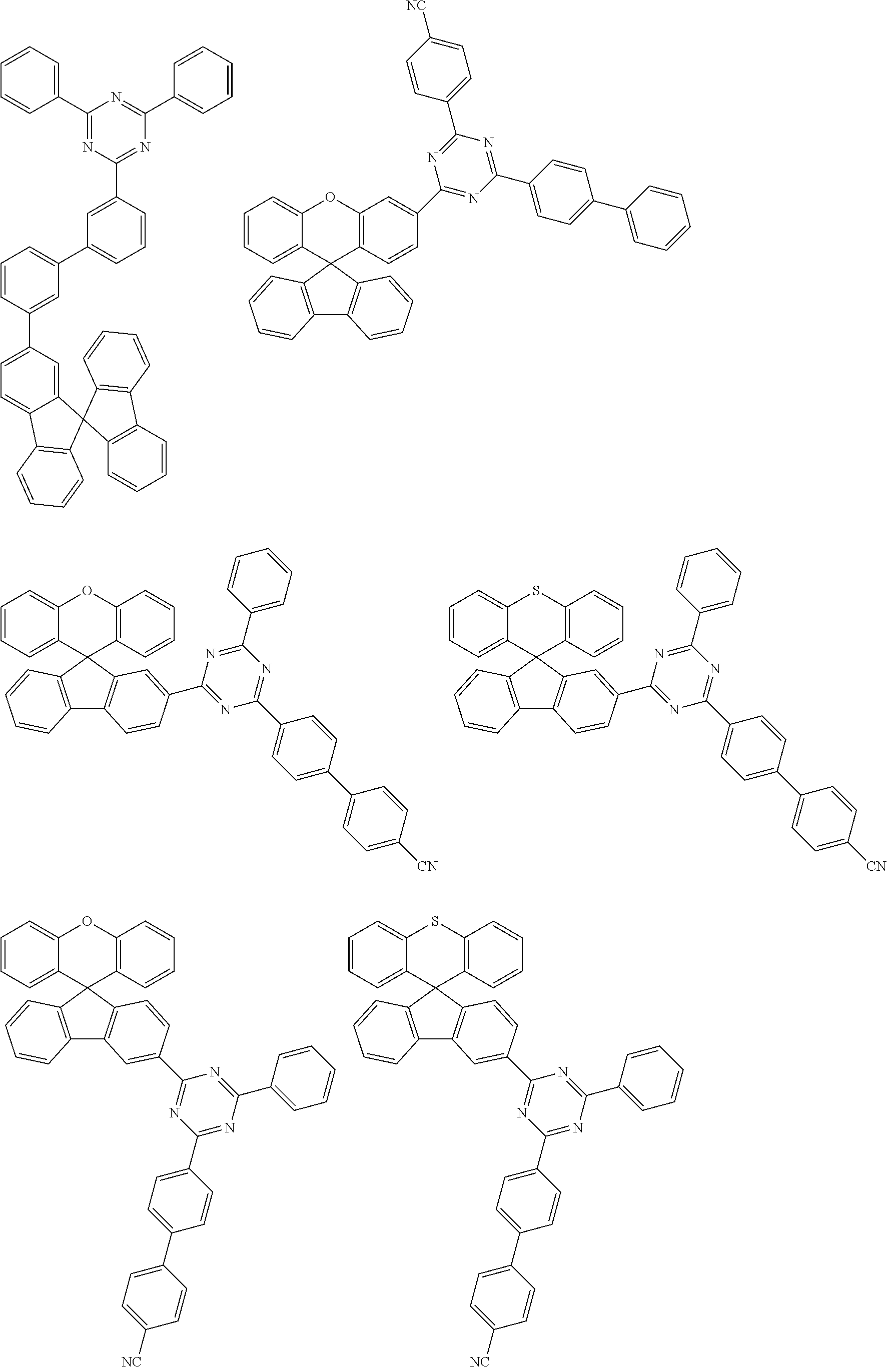

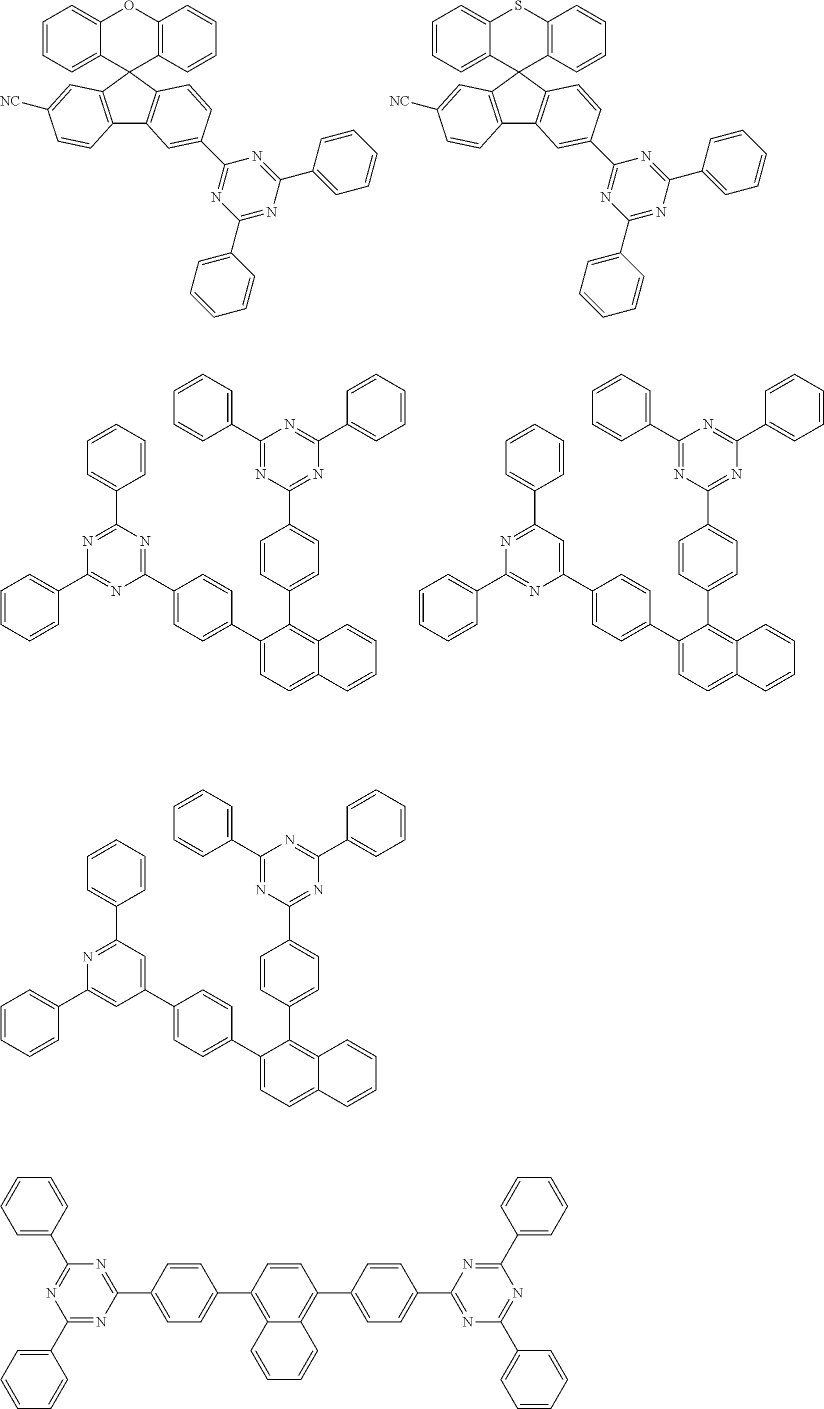

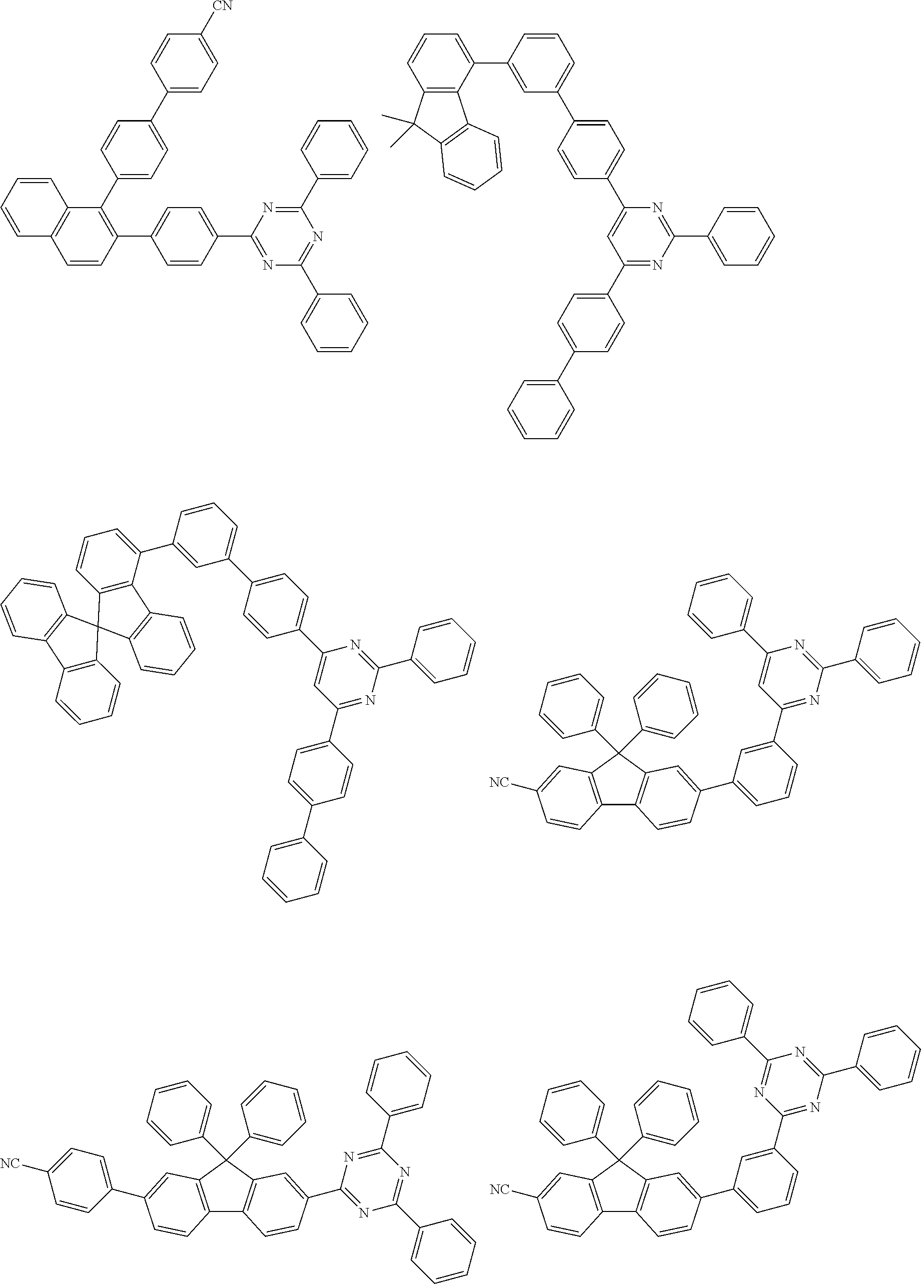

An embodiment may be an organic light emitting diode including the light emitting layer as the organic layer.

Another embodiment may be an organic light emitting diode including a light emitting layer and a hole transport region as the organic layer.

Another embodiment may be an organic light emitting diode including a light emitting layer and an electron transport region as the organic layer.

An organic light emitting diode according to an embodiment of the present invention may include a hole transport region 140 and an electron transport region 150 in addition to the light emitting layer 130 as the organic layer 105, as shown in FIG. 1.

On the other hand, an organic light emitting diode may further include an electron injection layer (not shown), a hole injection layer (not shown), etc. in addition to the light emitting layer as the organic layer.

The organic light emitting diodes 100 may be manufactured by forming an anode or a cathode on a substrate, and then forming an organic layer by a dry film method such as vacuum deposition, sputtering, plasma plating and ion plating, and forming a cathode or an anode thereon.

The organic light emitting diode may be applied to an organic light emitting display device.

### DETAILED DESCRIPTION OF THE EMBODIMENTS

Hereinafter, the embodiments are illustrated in more detail with reference to examples. However, these examples are exemplary, and the present scope is not limited thereto.

Hereinafter, starting materials and reactants used in Examples and Synthesis Examples were purchased from Sigma-Aldrich Co. Ltd., TCI Inc., Tokyo chemical industry, or P&H tech as far as there in no particular comment or were synthesized by known methods.

### (Preparation of Compound for Organic Optoelectronic Device)

The compounds presented as a more specific example of the compound of the present invention were synthesized through the following steps.

### Synthesis Example 1: Synthesis of Intermediate I-1

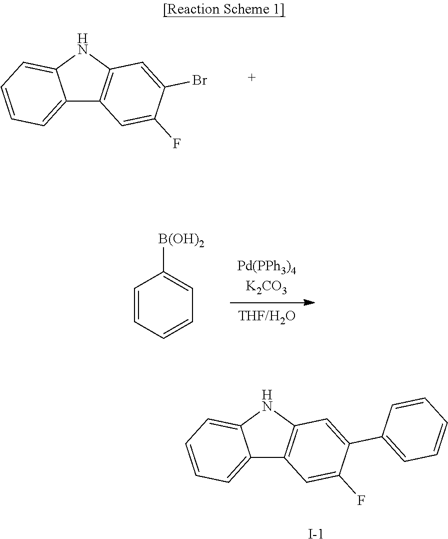 [SMILES: Fc1cc2c(cc1Br)[nH]c1ccccc12 ; OB(O)c1ccccc1 ; Fc1cc2c(cc1-c1ccccc1)[nH]c1ccccc12 ; [I-]]

In a nitrogen environment, 2-bromo-3-fluoro-9H-carbazole (100 g, 378 mmol), phenylboronic acid (55.4 g, 454 mmol) were added to a reactor, 600 ml of tetrahydrofuran was added thereto to dissolve them, and 300 ml of an aqueous solution in which K₂CO₃ (130 g, 946 mmol) was dissolved was added thereto and then, stirred. Subsequently, Pd(PPh₃)₄ (13 g, 11.4 mmol) was added thereto and then, heated 80° C. for 12 hours and stirred under reflux. When a reaction was completed, after adding water to the reaction solution, an extract was obtained therefrom by using dichloromethane, treated with magnesium sulfate anhydrous to remove moisture therefrom, filtered, and concentrated under a reduced pressure. The obtained residue was separated and purified through flash column chromatography to obtain Intermediate I-1 (79 g, 80%).

HRMS (70 eV, EI+): m/z calcd for C24H16N2: 261.0954, found: 261.

Elemental Analysis: C, 83%; H, 5%

### Synthesis Example 2: Synthesis of Intermediate I-2

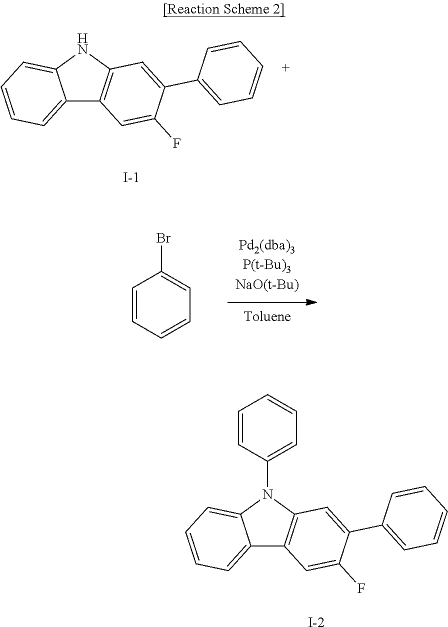

In a nitrogen environment, Intermediate I-1 (79 g, 303 mmol) and bromobenzene (57 g, 363 mmol) were added and dissolved in 1000 ml of toluene, and Pd₂(dba)₃ (13.8 g, 15.1 mmol), P(t-Bu)₃ (36.8 ml, 75.6 mmol), and sodium tert-butoxide (34.8 g, 362 mmol) were added thereto and then, heated for 12 hours and stirred under reflux. When a reaction was completed, water was added to the reaction solution to extract an organic layer, and the organic layer was treated with magnesium sulfate anhydrous to remove moisture, filtered, and concentrated under a reduced pressure. The obtained residue was separated and purified through flash column chromatography to obtain Intermediate I-2 (80 g, 78%).

HRMS (70 eV, EI+): m/z calcd for C24H16N2: 337.1267, found: 337.

Elemental Analysis: C, 85%; H, 5%

### Synthesis Example 3: Synthesis of Intermediate I-3

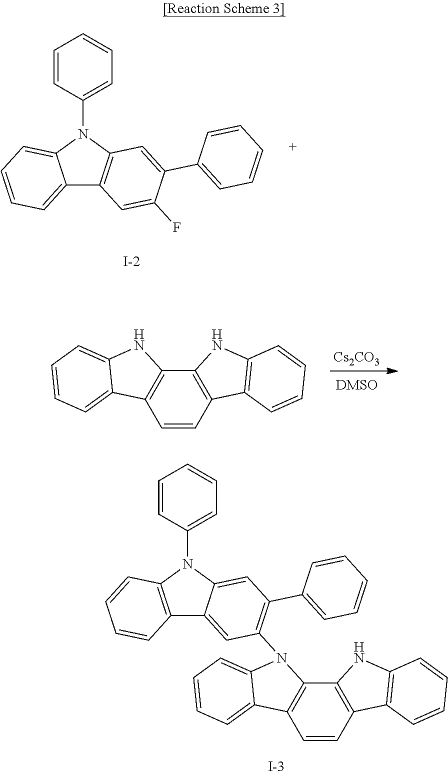

In a nitrogen environment, Intermediate I-2 (80 g, 237 mmol) and 11,12-dihydroindolo[2,3-a]carbazole (73 g, 284 mmol) were added and then, dissolved in 500 ml of dimethyl sulfoxide, and cesium carbonate (193 g, 592 mmol) was added thereto and then, heated for 12 hours and stirred under reflux. When a reaction was completed, water was added to the reaction solution to produce a solid, and the solid was separated by filtering under a reduced pressure and recrystallized by dissolving in toluene to obtain Intermediate I-3 (10 g, 7%).

HRMS (70 eV, EI+): m/z calcd for C24H16N2: 573.2205, found: 573.

Elemental Analysis: C, 88%; H, 5%

### Synthesis Example 4: Synthesis of Intermediate I-4

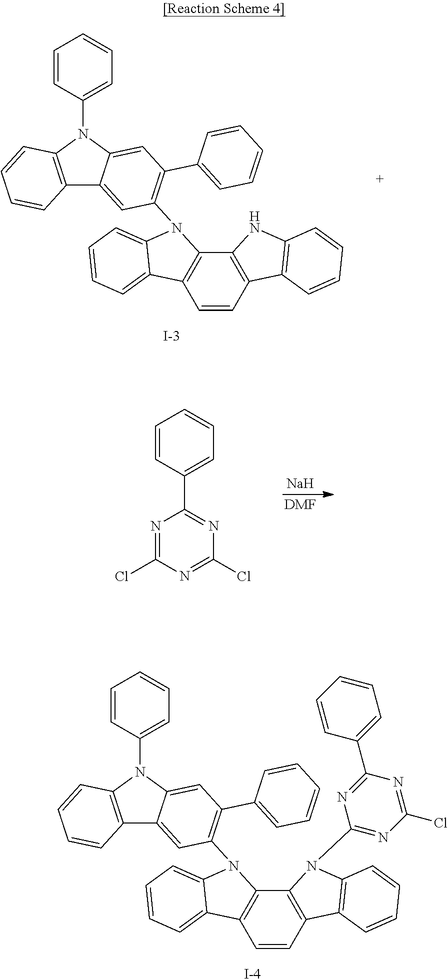

In a nitrogen environment, Intermediate I-3 (10 g, 17.4 mmol) was dissolved in DMF, and sodium hydride (0.63 g, 26.15 mmol) was added thereto and then, stirred for 30 minutes. Then, 2,4-dichloro-6-phenyl-1,3,5-triazine (7.9 g, 34.8 mmol) was added thereto and then, stirred at room temperature for 12 hours. When a reaction was completed, water was added to the reaction to produce a solid, and the solid was separated by filtering under a reduced pressure and recrystallized through dissolution in toluene to obtain Intermediate I-4 (9 g, 68%).

HRMS (70 eV, EI+): m/z calcd for C24H16N2: 762.2299, found: 762.

Elemental Analysis: C, 80%; H, 4%

### Synthesis Example 5: Synthesis of Compound A-2

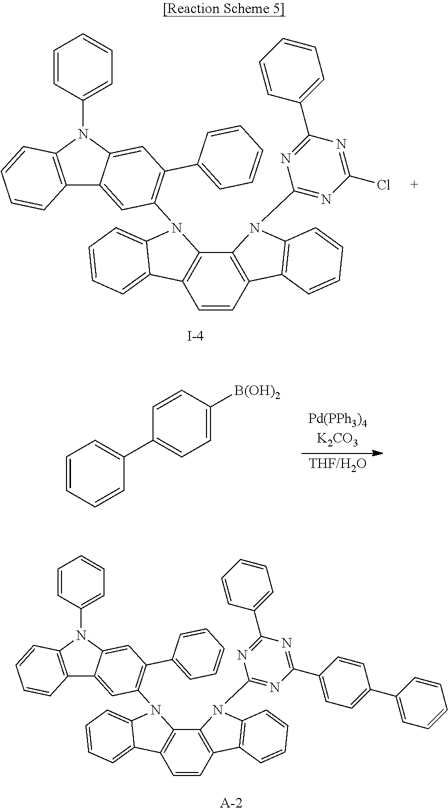

Compound A-2 (7 g, 67%) was obtained in the same manner as in Synthesis Example 1 except that Intermediate I-4 (9 g, 11.8 mmol) and [1,1′-biphenyl]-4-ylboronic acid (3.5 g, 17.7 mmol) were used.

HRMS (70 eV, EI+): m/z calcd for C27H18FN3: 880.3314, found: 880.

Elemental Analysis: C, 86%; H, 5%

### Synthesis Example 6: Synthesis of Compound A-73

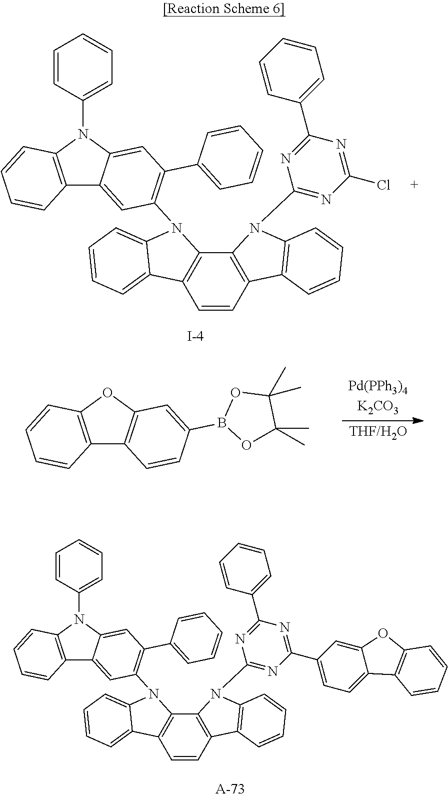

Compound A-73 (7.5 g, 71%) was obtained in the same manner as in Synthesis Example 1 except that Intermediate I-4 (9 g, 11.8 mmol) and 2-(dibenzo[b,d]furan-3-yl)-4,4,5,5-tetramethyl-1,3,2-dioxaborolane (5.2 g, 17.7 mmol) were used.

HRMS (70 eV, EI+): m/z calcd for C27H18FN3: 894.3107, found: 894.

Elemental Analysis: C, 85%; H, 4%

### Synthesis Example 7: Synthesis of Intermediate I-5

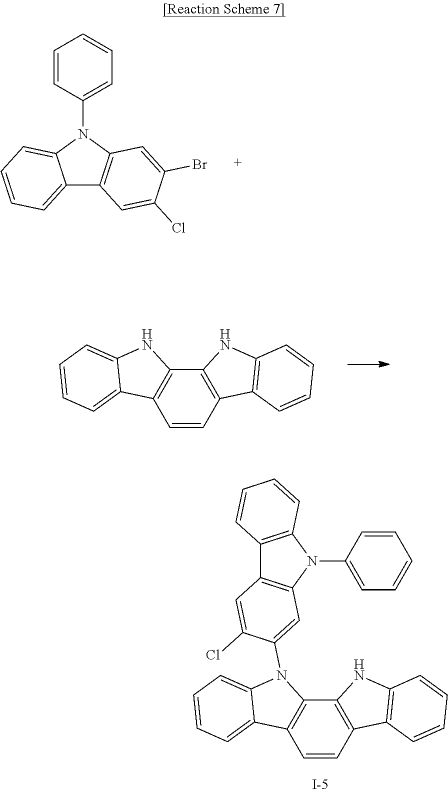

Intermediate I-5 (80 g, 54%) was obtained in the same manner as in Synthesis Example 2 except that 2-bromo-3-chloro-9-phenyl-9H-carbazole (100 g, 280 mmol) and 11,12-dihydroindolo[2,3-a]carbazole (108 g, 420 mmol) were used.

HRMS (70 eV, EI+): m/z calcd for C36H22ClN3: 531.1502, found: 531.

Elemental Analysis: C, 81%; H, 4%

### Synthesis Example 8: Synthesis of Intermediate I-6

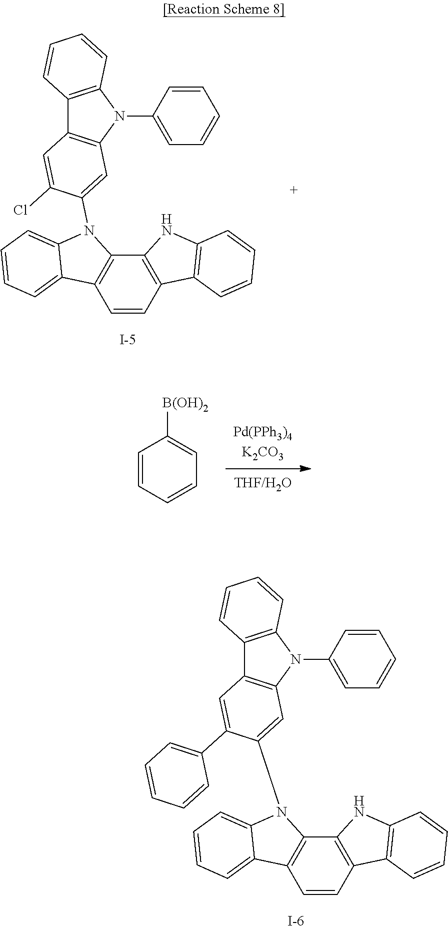

Intermediate I-6 (60 g, 72%) was obtained in the same manner as in Synthesis Example 1 except that Intermediate I-5 (80 g, 150 mmol) and phenylboronic acid (27.5 g, 225 mmol) were used.

HRMS (70 eV, EI+): m/z calcd for C42H27N3: 573.2205, found: 573.

Elemental Analysis: C, 88%; H, 5%

### Synthesis Example 9: Synthesis of Intermediate I-7

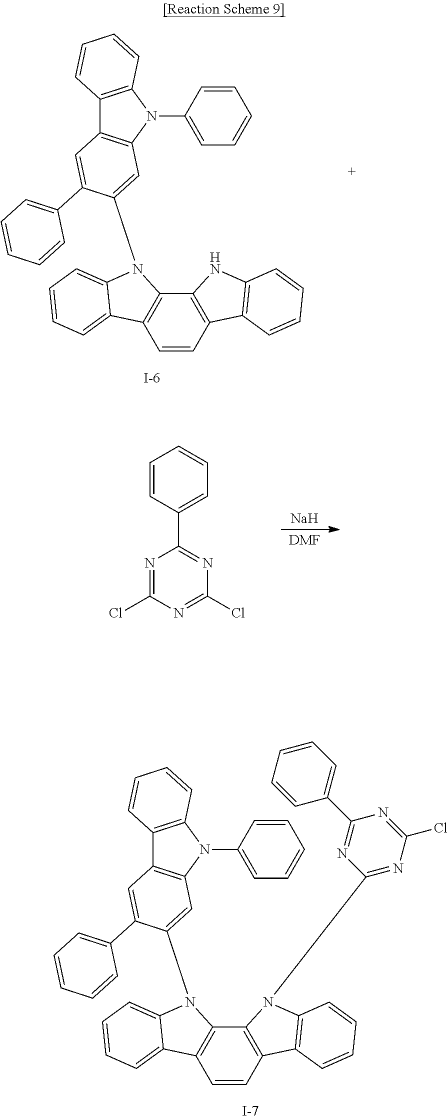

Intermediate I-7 (50 g, 63%) was obtained in the same manner as in Synthesis Example 4 except that Intermediate I-6 (60 g, 104 mmol) and 2,4-dichloro-6-phenyl-1,3,5-triazine (28.4 g, 125 mmol) were used.

HRMS (70 eV, EI+): m/z calcd for C51H31ClN6: 762.2299, found: 762.

Elemental Analysis: C, 80%; H, 4%

### Synthesis Example 10: Synthesis of Compound A-22

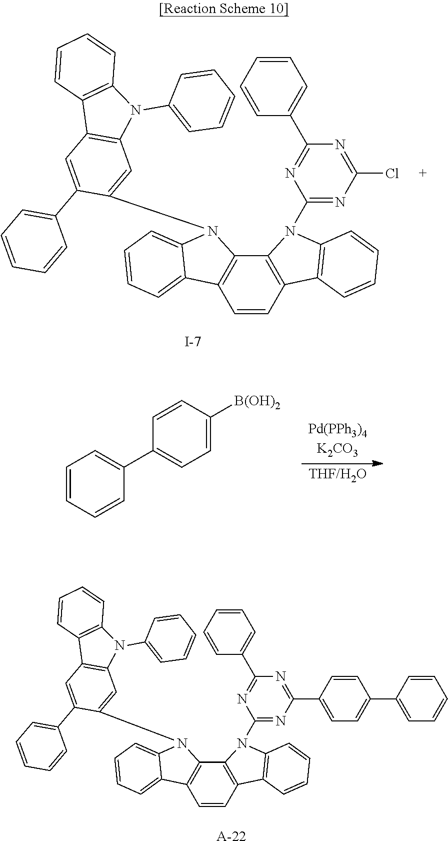

Compound A-22 (20 g, 35%) was obtained in the same manner as in Synthesis Example 1 except that Intermediate I-7 (50 g, 65.5 mmol) and [1,1′-biphenyl]-4-ylboronic acid (15.6 g, 78.6 mmol) were used.

HRMS (70 eV, EI+): m/z calcd for C63H40N6: 880.3314, found: 880.

Elemental Analysis: C, 86%; H, 5%

### Synthesis Example 11: Synthesis of Compound B-136

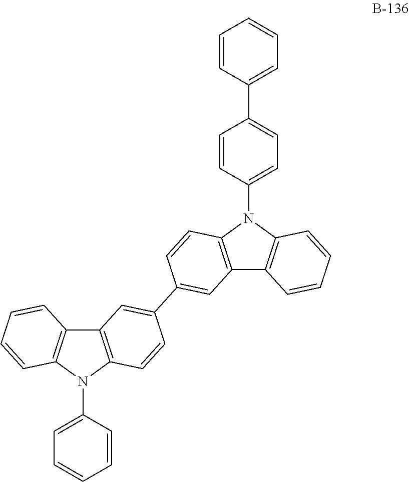 [SMILES: c1ccc(-c2ccc(-n3c4ccccc4c4cc(-c5ccc6c(c5)c5ccccc5n6-c5ccccc5)ccc43)cc2)cc1]

Compound B-136 was synthesized by referring to the synthesis method known in the U.S. Ser. No. 10/476,008 B2 registered patent.

HRMS (70 eV, EI+): m/z calcd for C42H28N2: 560.2252, found: 560.

Elemental Analysis: C, 90%; H, 5%

### Synthesis Example 12: Synthesis of Compound B-31

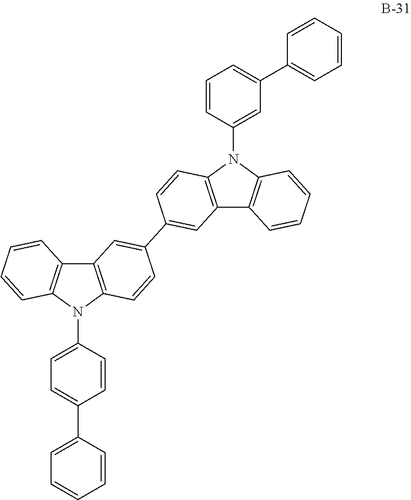 [SMILES: c1ccc(-c2ccc(-n3c4ccccc4c4cc(-c5ccc6c(c5)c5ccccc5n6-c5cccc(-c6ccccc6)c5)ccc43)cc2)cc1]

Compound B-31 was synthesized by referring to the synthesis method of patent EP2947071.

HRMS (70 eV, EI+): m/z calcd for C48H32N2: 636.2565, found: 636.

Elemental Analysis: C, 91%; H, 5%

### Synthesis Example 13: Synthesis of Compound B-99

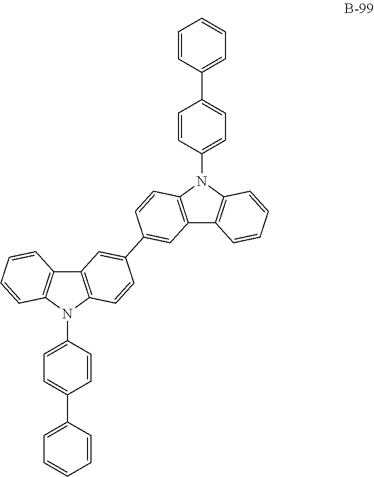 [SMILES: c1ccc(-c2ccc(-n3c4ccccc4c4cc(-c5ccc6c(c5)c5ccccc5n6-c5ccc(-c6ccccc6)cc5)ccc43)cc2)cc1]

Compound B-99 was synthesized by referring to the synthesis method of patent KR10-2019-0000597.

HRMS (70 eV, EI+): m/z calcd for C48H32N2: 636.2565, found: 636.

Elemental Analysis: C, 91%; H, 5%

### Synthesis Example 14: Synthesis of Compound C-4

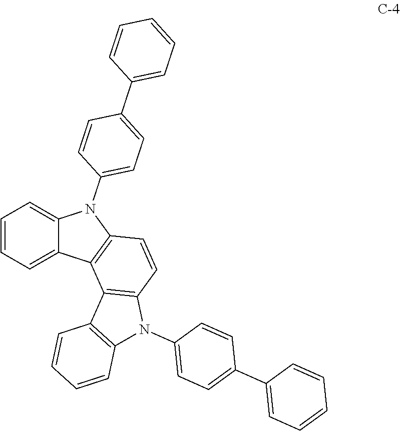 [SMILES: c1ccc(-c2ccc(-n3c4ccccc4c4c5c6ccccc6n(-c6ccc(-c7ccccc7)cc6)c5ccc43)cc2)cc1]

Compound C-4 was synthesized by referring to the synthesis method of patent KR2031300.

HRMS (70 eV, EI+): m/z calcd for C42H28N2: 560.2252, found: 560.

Elemental Analysis: C, 90%; H, 5%

### Synthesis Example 15: Synthesis of Compound C-57

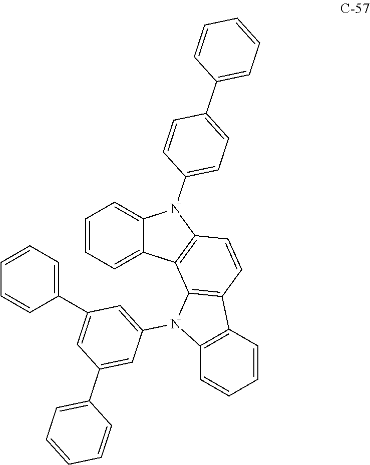 [SMILES: c1ccc(-c2ccc(-n3c4ccccc4c4c3ccc3c5ccccc5n(-c5cc(-c6ccccc6)cc(-c6ccccc6)c5)c34)cc2)cc1]

Compound C-57 was synthesized by referring to the synthesis method of patent WO2018-095391.

HRMS (70 eV, EI+): m/z calcd for C48H32N2: 636.2565, found: 636.

Elemental Analysis: C, 91%; H, 5%

### Synthesis Example 16: Synthesis of Compound R-1

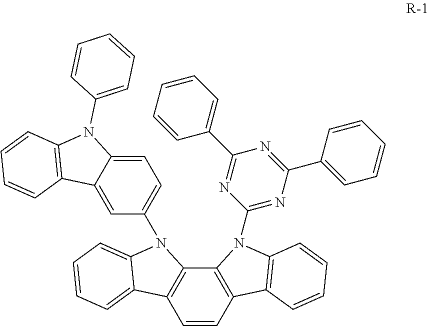 [SMILES: c1ccc(-c2nc(-c3ccccc3)nc(-n3c4ccccc4c4ccc5c6ccccc6n(-c6ccc7c(c6)c6ccccc6n7-c6ccccc6)c5c43)n2)cc1]

Compound R-1 was synthesized by referring to the synthesis method known in the KR 10-2014-0113672 A registered patent.

HRMS (70 eV, EI+): m/z calcd for C51H32N6: 728.2688, found: 728.

Elemental Analysis: C, 84%; H, 4%

### Synthesis Example 17: Synthesis of Compound R-2

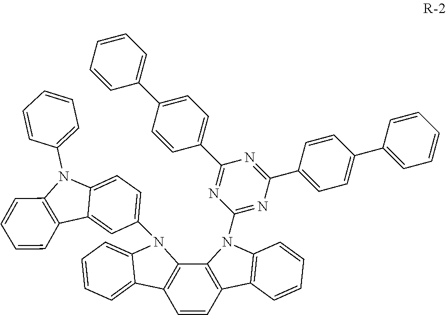 [SMILES: c1ccc(-c2ccc(-c3nc(-c4ccc(-c5ccccc5)cc4)nc(-n4c5ccccc5c5ccc6c7ccccc7n(-c7ccc8c(c7)c7ccccc7n8-c7ccccc7)c6c54)n3)cc2)cc1]

Compound R-2 was synthesized by referring to the synthesis method known in the KR 10-2014-0113672 A registered patent.

HRMS (70 eV, EI+): m/z calcd for C63H40N6: 880.3314, found: 880.

Elemental Analysis: C, 86%; H, 5%

### Manufacture of Organic Light Emitting Diode (Green)

### Example 1

A glass substrate coated with a thin film of ITO (indium tin oxide) was ultrasonically washed with distilled water. After washing with the distilled water, the glass substrate was washed with a solvent such as isopropyl alcohol, acetone, methanol, and the like ultrasonically and dried and then, moved to a plasma cleaner, cleaned by using oxygen plasma for 10 minutes, and moved to a vacuum depositor. This prepared ITO transparent electrode was used as an anode, Compound A doped with 3% NDP-9 (Novaled GmbH) was vacuum-deposited on the ITO substrate to form a 100 Å-thick hole injection layer, and Compound A is deposited on the hole injection layer to a thickness of 1350 Å to form a hole transport layer. On the hole transport layer, Compound B was deposited to a thickness of 350 Å to form a hole transport auxiliary layer, and on the hole transport auxiliary layer, Compound A-2 synthesized in Synthesis Example 5 was used as a host, and PhGD was doped at 7 wt % as a dopant to form a 400 Å-thick light emitting layer by vacuum deposition, and in the following examples and comparative examples, ratios are described separately. Subsequently, Compound C was deposited on the light emitting layer to a thickness of 50 Å to form an electron transport auxiliary layer, and Compound D and Liq were simultaneously vacuum-deposited at a weight ratio of 1:1 to form an electron transport layer with a thickness of 300 Å. An organic light emitting diode was manufactured by sequentially vacuum depositing 15 Å of Liq and 1200 Å of Al on the electron transport layer to form a cathode.

TO/Compound A (3% NDP-9 doping, 100 Å)/Compound A (1350 Å)/Compound B (350 Å)/EML [Compound A-2: PhGD (7 wt %)](400 Å)/Compound C (50 Å)/Compound D: LiQ (300 Å)/LiQ (15 Å)/Al (1200 Å).
Compound A: N-(biphenyl-4-yl)-9,9-dimethyl-N-(4-(9-phenyl-9H-carbazol-3-yl)phenyl)-9H-fluoren-2-amine
Compound B: N-[4-(4-Dibenzofuranyl)phenyl]-N-[4-(9-phenyl-9H-fluoren-9-yl)phenyl][1,1′-biphenyl]-4-amine
Compound C: 2,4-Diphenyl-6-(4′,5′,6′-triphenyl[1,1′:2′,1″:3″,1′″:3′″,1″″-quinquephenyl]-3″″-yl)-1,3,5-triazine
Compound D: 2-(1,1′-Biphenyl-4-yl)-4-(9,9-diphenylfluoren-4-yl)-6-phenyl-1,3,5-triazine

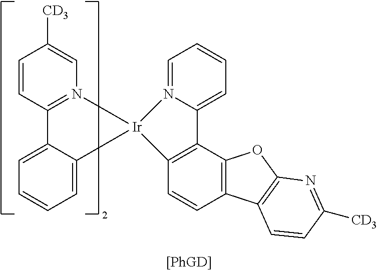

### Examples 2 to 3 and Comparative Examples 1 to 2

Each organic light emitting diode was manufactured in the same manner as in Example 1, except that the composition was changed to those shown in Table 1.

### Example 4

A glass substrate coated with a thin film of ITO (indium tin oxide) was ultrasonically washed with distilled water. After washing with the distilled water, the glass substrate was washed with a solvent such as isopropyl alcohol, acetone, methanol, and the like ultrasonically and dried and then, moved to a plasma cleaner, cleaned by using oxygen plasma for 10 minutes, and moved to a vacuum depositor. This prepared ITO transparent electrode was used as an anode, Compound A doped with 3% NDP-9 (Novaled GmbH) was vacuum-deposited on the ITO substrate to form a 100 Å-thick hole injection layer, and Compound A is deposited on the hole injection layer to a thickness of 1350 Å to form a hole transport layer. Compound E was deposited on the hole transport layer to a thickness of 350 Å to form a hole transport auxiliary layer. On the hole transport auxiliary layer, Compound A-2 synthesized in Synthesis Example 5 and Compound B-136 synthesized in Synthesis Example 11 were used as hosts, and PhGD was doped at 10 wt % as a dopant to form a 400 Å-thick light emitting layer by vacuum deposition. Herein, Compound A-1 and Compound B-136 were used at a weight ratio of 3:7. Subsequently, Compound F was deposited on the emitting layer to a thickness of 50 Å to form an electron transport auxiliary layer, and Compound G and Liq were simultaneously vacuum-deposited at a weight ratio of 1:1 to form a 300 Å-thick electron transport layer with a thickness of 300 Å. An organic light emitting diode was manufactured by sequentially vacuum depositing 15 Å of LiQ and 1200 Å of Al on the electron transport layer to form a cathode.

ITO/Compound A (3% NDP-9 doping, 100 Å)/Compound A (1350 Å)/Compound E (350 Å)/EML [Compound A-2: Compound B-136:PhGD=27:63:10 wt %)](400 Å)/Compound F (50 Å)/Compound G:LiQ (300 Å)/LiQ (15 Å)/Al (1200 Å).
Compound E: N,N-bis(9,9-dimethyl-9H-fluoren-4-yl)-9,9-spirobi(fluorene)-2-amine
Compound F: 2-[3′-(9,9-Dimethyl-9H-fluoren-2-yl)[1,1′-biphenyl]-3-yl]-4,6-diphenyl-1,3,5-triazine
Compound G: 2-[4-[4-(4′-Cyano-1,1′-biphenyl-4-yl)-1-naphthyl]phenyl]-4,6-diphenyl-1,3,5-triazine

### Examples 5 to 10 and Comparative Examples 3 to 4

Each organic light emitting diode was manufactured in the same manner as in Example 4, except that the composition was changed to those shown in Table 2.

### Example 11

An organic light emitting diode was manufactured in the same manner as in Example 5, except that Compound A-22 and Compound B-136 were used in a weight ratio of 4:6.

### Example 12

An organic light emitting diode was manufactured in the same manner as in Example 5, except that Compound A-22 and Compound B-136 were used in a weight ratio of 2:8.

### Evaluation

The driving voltage, luminous efficiency, and life-span characteristics of the organic light emitting diodes according to Examples 1 to 12 and Comparative Examples 1 to 4 were evaluated.

The specific measurement method is as follows, and the results are as shown in Tables 1 and 2.

### (1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

### (2) Measurement of Luminance Change Depending on Voltage Change

Luminance was measured by using a luminance meter (Minolta Cs-1000A), while the voltage of the organic light emitting diodes was increased from 0 V to 10 V.

### (3) Measurement of Luminous Efficiency

Current efficiency (cd/A) at the same current density (10 mA/cm²) were calculated by using the luminance and current density from (1) and (2) above and voltage.

The relative values of luminous efficiency based on Comparative Example 1 are shown in Table 1.

The relative values of luminous efficiency based on Comparative Example 3 are shown in Table 2.

### (4) Measurement of Life-Span

The results were obtained by maintaining the luminance (cd/m²) at 24000 cd/m² and measuring the time for the current efficiency (cd/A) to decrease to 97%.

The relative values of life-span based on Comparative Example 1 are shown in Table 1.

The relative values of life-span based on Comparative Example 3 are shown in Table 2.

### (5) Measurement of Driving Voltage

The results were obtained by measuring the driving voltage of each device at 15 mA/cm² using a current-voltage meter (Keithley 2400).

The relative values of the driving voltage based on Comparative Example 1 are shown in Table 1.

The relative values of the driving voltage based on Comparative Example 3 are shown in Table 2.

TABLE 1

| | | Driving | Color | Efficiency | Life-span |
|---|---|---|---|---|---|
| No. | Compound | voltage (%) | (EL color) | (%) | (%) |
| Example 1 | A-2 | 97% | Green | 109% | 114% |
| Example 2 | A-22 | 95% | Green | 115% | 142% |
| Example 3 | A-73 | 96% | Green | 118% | 128% |
| Comparative Example 1 | R-1 | 100% | Green | 100% | 100% |
| Comparative Example 2 | R-2 | 98% | Green | 103% | 85% |

TABLE 2

| | | Driving | | | |
|---|---|---|---|---|---|
| | | voltage | Color | Efficiency | Life-span |
| No. | Compound | (%) | (EL color) | (%) | (%) |
| Example 4 | A-2/B-136 | 98% | Green | 108% | 120% |
| Example 5 | A-22/B-136 | 95% | Green | 112% | 130% |
| Example 6 | A-73/B-136 | 96% | Green | 104% | 117% |
| Example 7 | A-22/B-31 | 99% | Green | 110% | 119% |
| Example 8 | A-22/B-99 | 98% | Green | 106% | 122% |
| Example 9 | A-22/C-4 | 96% | Green | 110% | 113% |
| Example 10 | A-22/C-57 | 97% | Green | 109% | 117% |
| Example 11 | A-22/B-136 | 94% | Green | 109% | 107% |
| Example 12 | A-22/B-136 | 99% | Green | 112% | 126% |
| Comparative | R-1/B-136 | 100% | Green | 100% | 100% |
| Example 3 | | | | | |
| Comparative | R-2/B-136 | 100% | Green | 102% | 111% |
| Example 4 | | | | | |

Referring to Table 1, the organic light emitting diodes manufactured by respectively applying the compounds according to the examples, compared with the organic light emitting diodes manufactured by respectively applying the compounds according to the comparative examples, exhibited significantly improved driving voltage, efficiency, and life-span characteristics. In particular, referring to Table 2, the organic light emitting diodes manufactured by applying compositions respectively including the compounds according to the examples also exhibited improved efficiency and life-span characteristics.

While this invention has been described in connection with what is presently considered to be practical example embodiments, it is to be understood that the invention is not limited to the disclosed embodiments, but, on the contrary, is intended to cover various modifications and equivalent arrangements included within the spirit and scope of the appended claims.

1. A compound for an organic optoelectronic device, the compound being represented by a combination of Chemical Formula 1 and Chemical Formula 2: 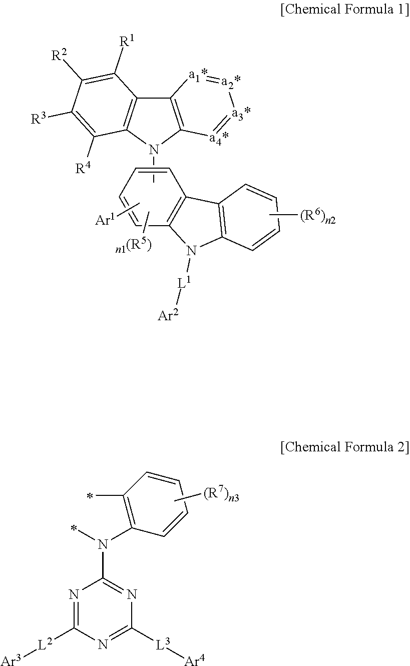
in Chemical Formulas 1 and 2,
Ar¹ is a substituted or unsubstituted C6 to C30 aryl group, a substituted or unsubstituted dibenzofuranyl group, a substituted or unsubstituted dibenzothiophenyl group, or a substituted or unsubstituted carbazolyl group,
Ar² to Ar⁴ are each independently a substituted or unsubstituted C6 to C20 aryl group or a substituted or unsubstituted C2 to C30 heterocyclic group,
two adjacent ones of a1* to a4* of Chemical Formula 1 are linking carbons linked at * of Chemical Formula 2,
the remaining two of a1* to a4* of Chemical Formula 1, not linked at * Chemical Formula 2 are each independently C-L^{a}-R^{a},
L^{a}, and L¹ to L³ are each independently a single bond or a substituted or unsubstituted C6 to C20 arylene group,
R^{a} and R¹ to R⁷ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
n1 is an integer of 1 or 2, and
n2 and n3 are each independently an integer of 1 to 4.

2. The compound for an organic optoelectronic device as claimed in claim 1, wherein:
Chemical Formula 1 is represented by one of Chemical Formula 1-1 to Chemical Formula 1-4: 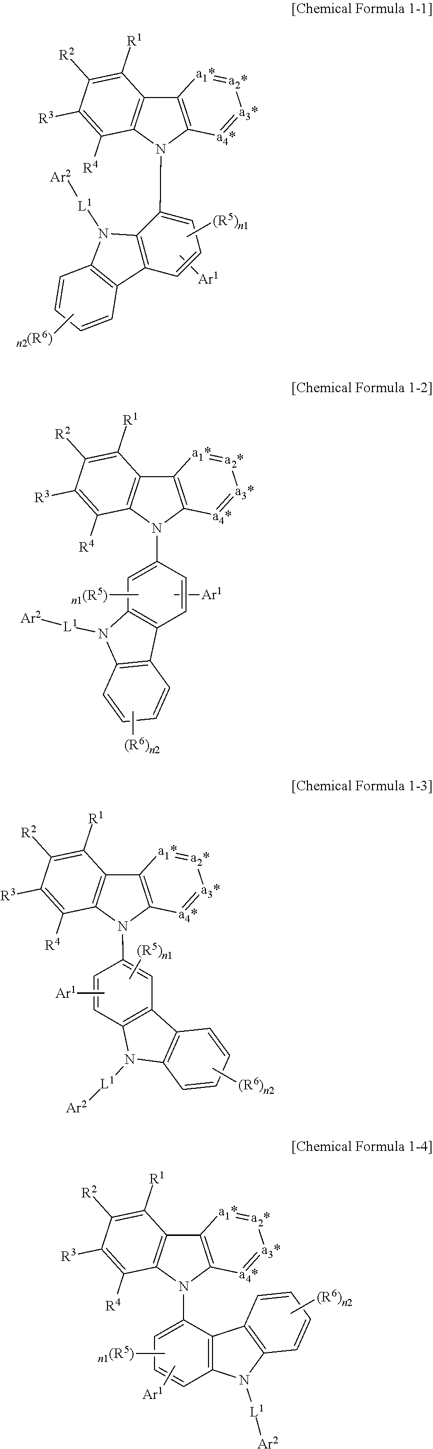
in Chemical Formula 1-1 to Chemical Formula 1-4, Ar¹, Ar², R¹ to R⁶, L¹, n1 and n2, and a1* to a4* are defined the same as those of Chemical Formulas 1 and 2.

3. The compound for an organic optoelectronic device as claimed in claim 1, wherein:
the compound is represented by one of Chemical Formula 1A to Chemical Formula 1F: 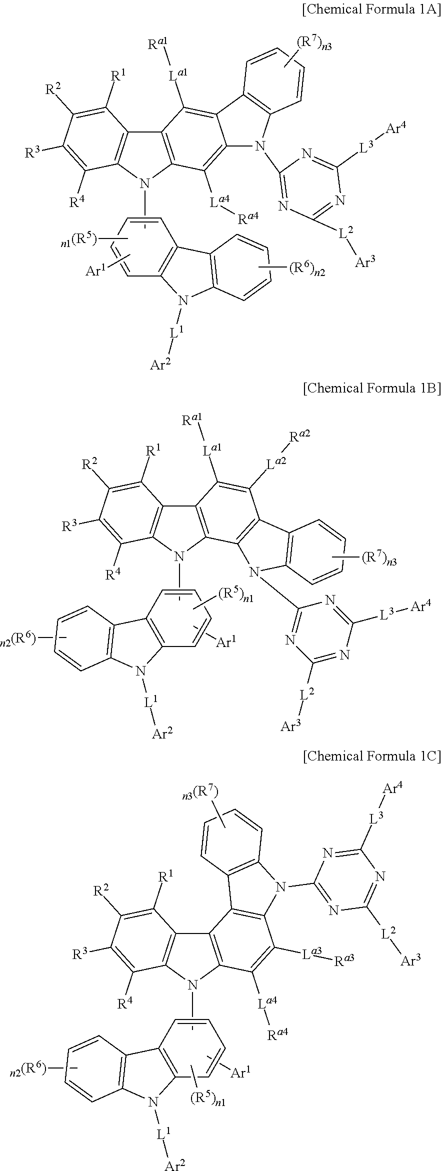 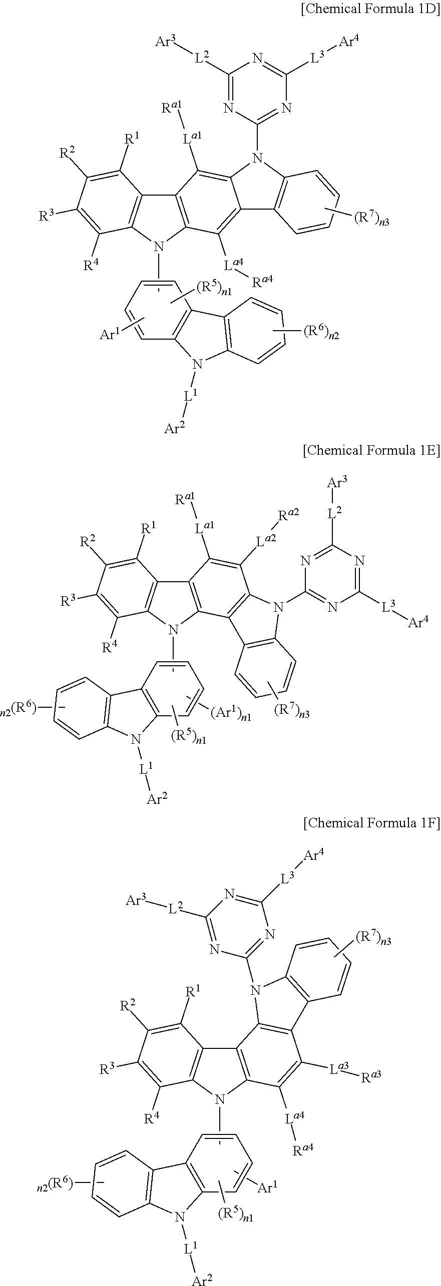
in Chemical Formula 1A to Chemical Formula 1F,
Ar¹ to Ar⁴, R¹ to R⁷, L¹ to L³, and n1 to n3 are defined the same as those of Chemical Formulas 1 and 2,
L^{a1} to L^{a4} are each independently defined the same as L^{a} of Chemical Formulas 1 and 2, and
R^{a1} to R^{a4} are each independently defined the same as R^{a} of Chemical Formulas 1 and 2.

4. The compound for an organic optoelectronic device as claimed in claim 3, wherein:
the compound is represented by one of Chemical Formula 1-2B, Chemical Formula 1-3B, Chemical Formula 1-2E, and Chemical Formula 1-3E: 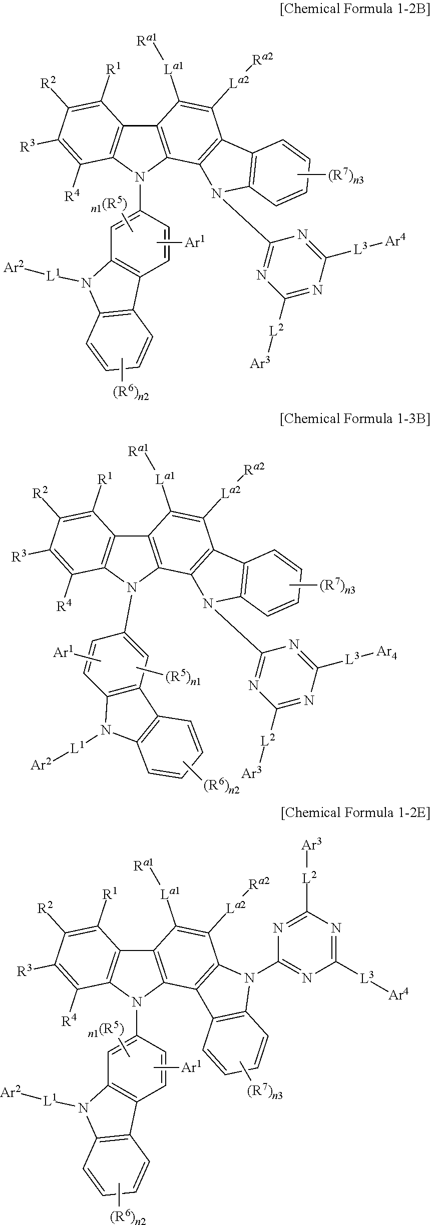 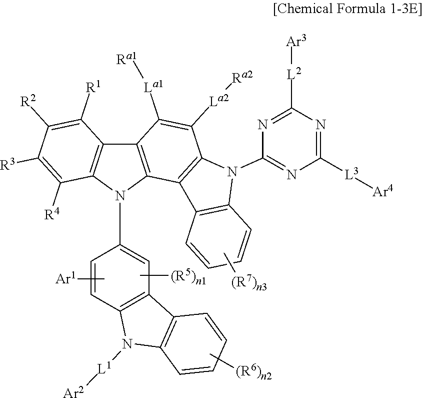
in Chemical Formula 1-2B, Chemical Formula 1-3B, Chemical Formula 1-2E, and Chemical Formula 1-3E, Ar¹ to Ar³, R¹ to R⁷, R^{a1}, R², L¹ to L³, L^{a1}, L^{a2}, and n1 to n3 are defined the same as those of Chemical Formula 1A to Chemical Formula 1F.

5. The compound for an organic optoelectronic device as claimed in claim 1, wherein Ar¹ is a substituted or unsubstituted C6 to C20 aryl group, a substituted or unsubstituted dibenzofuranyl group, a substituted or unsubstituted dibenzothiophenyl group, or a substituted or unsubstituted carbazolyl group.

6. The compound for an organic optoelectronic device as claimed in claim 1, wherein Ar³ and Ar⁴ are each independently a substituted or unsubstituted phenyl group, a substituted or unsubstituted biphenyl group, a substituted or unsubstituted naphthyl group, a substituted or unsubstituted triphenylene group, a substituted or unsubstituted fluorenyl group, a substituted or unsubstituted dibenzofuranyl group, a substituted or unsubstituted dibenzothiophenyl group, or a substituted or unsubstituted dibenzosilolyl group.

7. The compound for an organic optoelectronic device as claimed in claim 1, wherein:
moieties *-L²-Ar³ and L³-Ar⁴ are each independently a moiety of Group I: 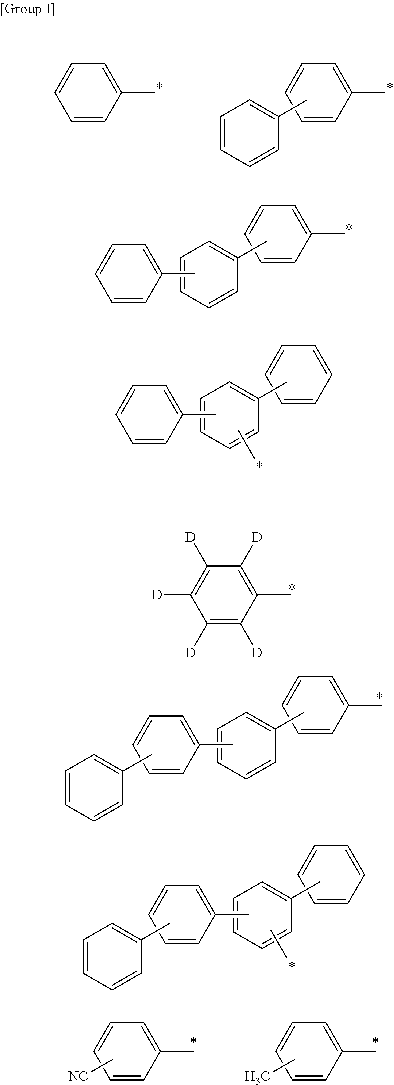 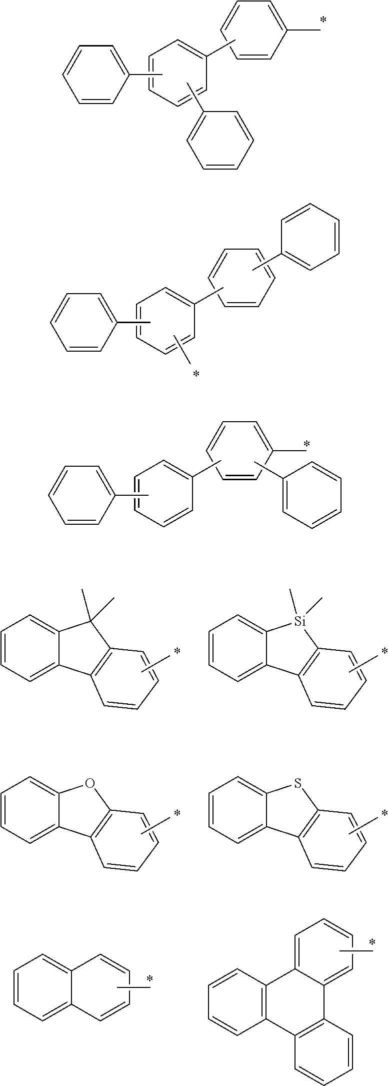
in Group I, * is a linking point.

8. The compound for an organic optoelectronic device as claimed in claim 1, wherein the compound is a compound of Group 1:

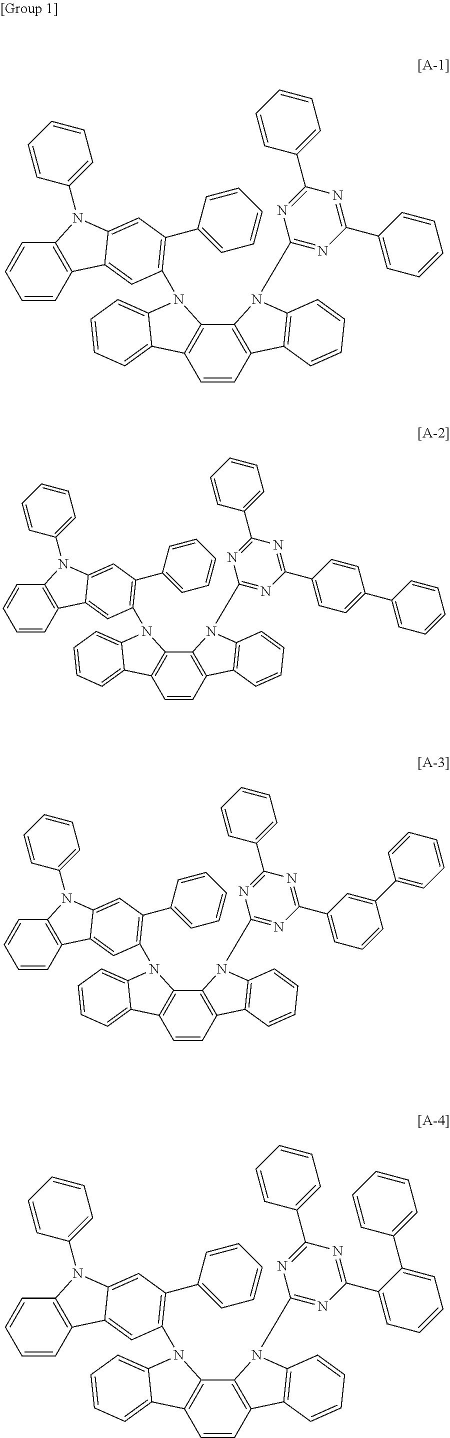 [SMILES: c1ccc(-c2nc(-c3ccccc3)nc(-n3c4ccccc4c4ccc5c6ccccc6n(-c6cc7c8ccccc8n(-c8ccccc8)c7cc6-c6ccccc6)c5c43)n2)cc1 ; c1ccc(-c2ccc(-c3nc(-c4ccccc4)nc(-n4c5ccccc5c5ccc6c7ccccc7n(-c7cc8c9ccccc9n(-c9ccccc9)c8cc7-c7ccccc7)c6c54)n3)cc2)cc1 ; c1ccc(-c2cccc(-c3nc(-c4ccccc4)nc(-n4c5ccccc5c5ccc6c7ccccc7n(-c7cc8c9ccccc9n(-c9ccccc9)c8cc7-c7ccccc7)c6c54)n3)c2)cc1 ; c1ccc(-c2nc(-c3ccccc3-c3ccccc3)nc(-n3c4ccccc4c4ccc5c6ccccc6n(-c6cc7c8ccccc8n(-c8ccccc8)c7cc6-c6ccccc6)c5c43)n2)cc1]

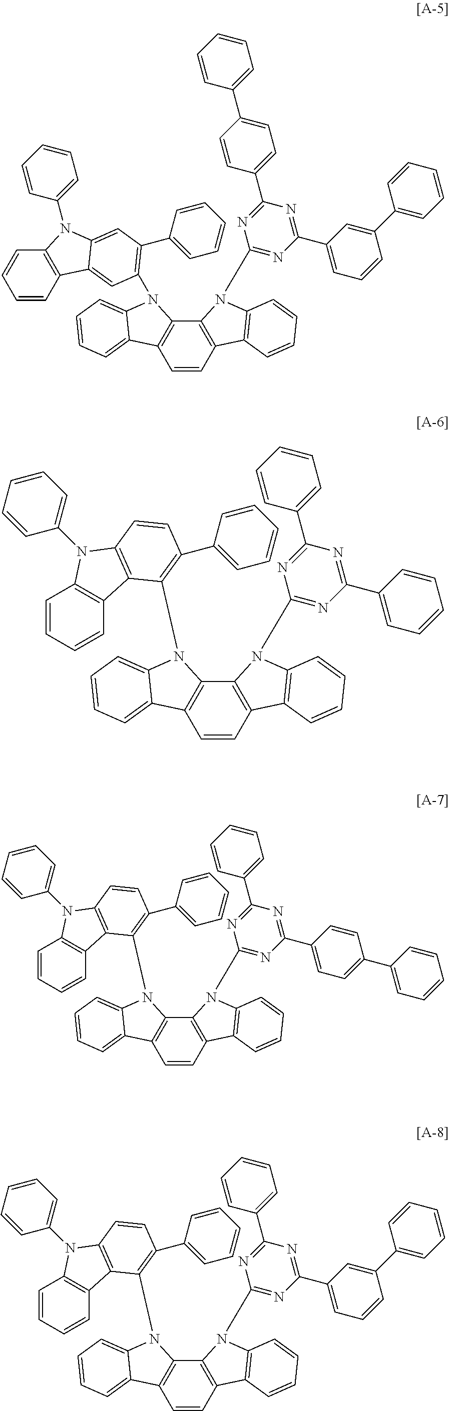 [SMILES: c1ccc(-c2ccc(-c3nc(-c4cccc(-c5ccccc5)c4)nc(-n4c5ccccc5c5ccc6c7ccccc7n(-c7cc8c9ccccc9n(-c9ccccc9)c8cc7-c7ccccc7)c6c54)n3)cc2)cc1 ; c1ccc(-c2nc(-c3ccccc3)nc(-n3c4ccccc4c4ccc5c6ccccc6n(-c6c(-c7ccccc7)ccc7c6c6ccccc6n7-c6ccccc6)c5c43)n2)cc1 ; c1ccc(-c2ccc(-c3nc(-c4ccccc4)nc(-n4c5ccccc5c5ccc6c7ccccc7n(-c7c(-c8ccccc8)ccc8c7c7ccccc7n8-c7ccccc7)c6c54)n3)cc2)cc1 ; c1ccc(-c2cccc(-c3nc(-c4ccccc4)nc(-n4c5ccccc5c5ccc6c7ccccc7n(-c7c(-c8ccccc8)ccc8c7c7ccccc7n8-c7ccccc7)c6c54)n3)c2)cc1]

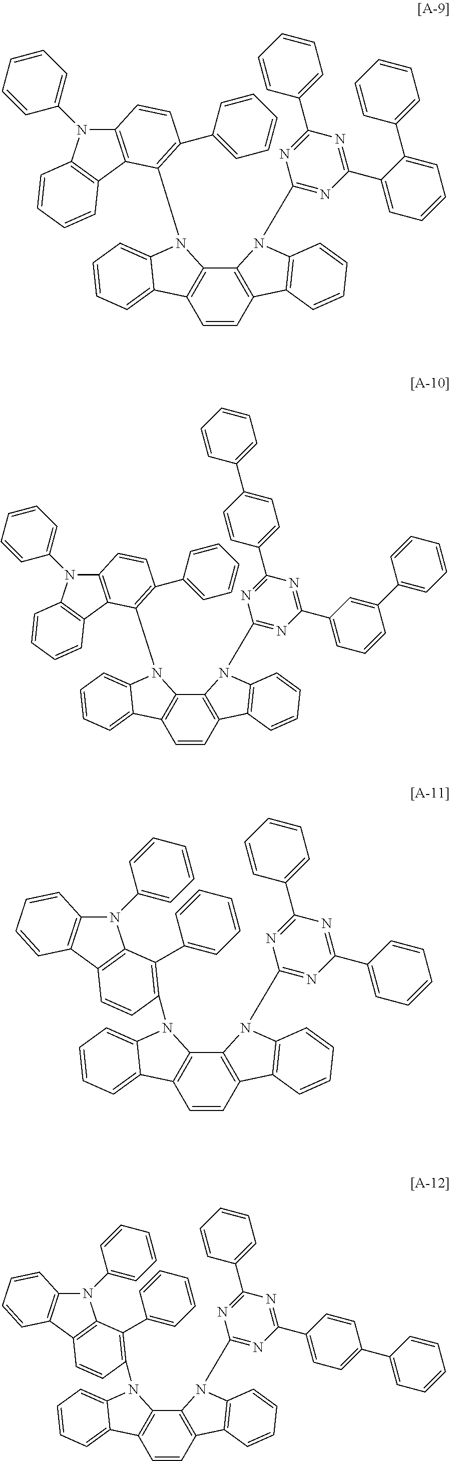 [SMILES: c1ccc(-c2nc(-c3ccccc3-c3ccccc3)nc(-n3c4ccccc4c4ccc5c6ccccc6n(-c6c(-c7ccccc7)ccc7c6c6ccccc6n7-c6ccccc6)c5c43)n2)cc1 ; c1ccc(-c2ccc(-c3nc(-c4cccc(-c5ccccc5)c4)nc(-n4c5ccccc5c5ccc6c7ccccc7n(-c7c(-c8ccccc8)ccc8c7c7ccccc7n8-c7ccccc7)c6c54)n3)cc2)cc1 ; c1ccc(-c2nc(-c3ccccc3)nc(-n3c4ccccc4c4ccc5c6ccccc6n(-c6ccc7c8ccccc8n(-c8ccccc8)c7c6-c6ccccc6)c5c43)n2)cc1 ; c1ccc(-c2ccc(-c3nc(-c4ccccc4)nc(-n4c5ccccc5c5ccc6c7ccccc7n(-c7ccc8c9ccccc9n(-c9ccccc9)c8c7-c7ccccc7)c6c54)n3)cc2)cc1]

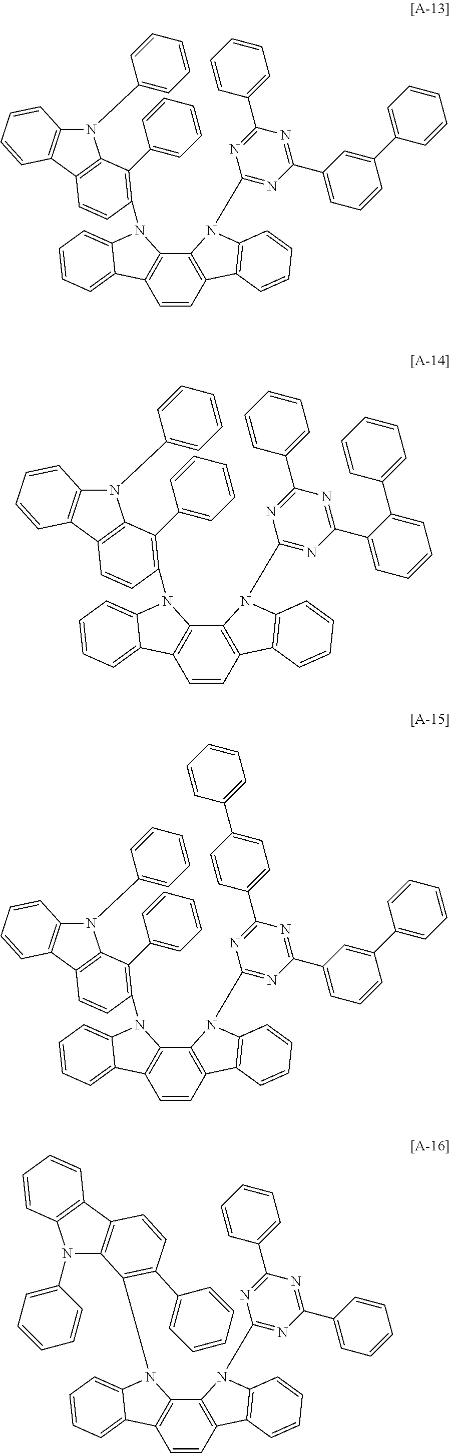 [SMILES: c1ccc(-c2cccc(-c3nc(-c4ccccc4)nc(-n4c5ccccc5c5ccc6c7ccccc7n(-c7ccc8c9ccccc9n(-c9ccccc9)c8c7-c7ccccc7)c6c54)n3)c2)cc1 ; c1ccc(-c2nc(-c3ccccc3-c3ccccc3)nc(-n3c4ccccc4c4ccc5c6ccccc6n(-c6ccc7c8ccccc8n(-c8ccccc8)c7c6-c6ccccc6)c5c43)n2)cc1 ; c1ccc(-c2ccc(-c3nc(-c4cccc(-c5ccccc5)c4)nc(-n4c5ccccc5c5ccc6c7ccccc7n(-c7ccc8c9ccccc9n(-c9ccccc9)c8c7-c7ccccc7)c6c54)n3)cc2)cc1 ; c1ccc(-c2nc(-c3ccccc3)nc(-n3c4ccccc4c4ccc5c6ccccc6n(-c6c(-c7ccccc7)ccc7c8ccccc8n(-c8ccccc8)c67)c5c43)n2)cc1]

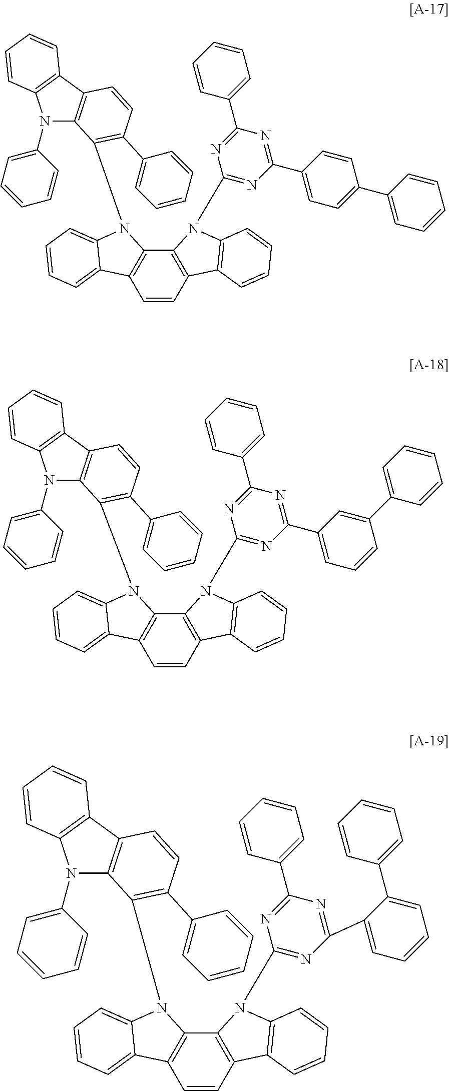 [SMILES: c1ccc(-c2ccc(-c3nc(-c4ccccc4)nc(-n4c5ccccc5c5ccc6c7ccccc7n(-c7c(-c8ccccc8)ccc8c9ccccc9n(-c9ccccc9)c78)c6c54)n3)cc2)cc1 ; c1ccc(-c2cccc(-c3nc(-c4ccccc4)nc(-n4c5ccccc5c5ccc6c7ccccc7n(-c7c(-c8ccccc8)ccc8c9ccccc9n(-c9ccccc9)c78)c6c54)n3)c2)cc1 ; c1ccc(-c2nc(-c3ccccc3-c3ccccc3)nc(-n3c4ccccc4c4ccc5c6ccccc6n(-c6c(-c7ccccc7)ccc7c8ccccc8n(-c8ccccc8)c67)c5c43)n2)cc1]

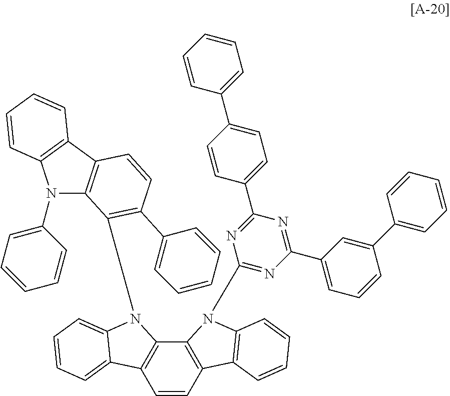 [SMILES: c1ccc(-c2ccc(-c3nc(-c4cccc(-c5ccccc5)c4)nc(-n4c5ccccc5c5ccc6c7ccccc7n(-c7c(-c8ccccc8)ccc8c9ccccc9n(-c9ccccc9)c78)c6c54)n3)cc2)cc1]

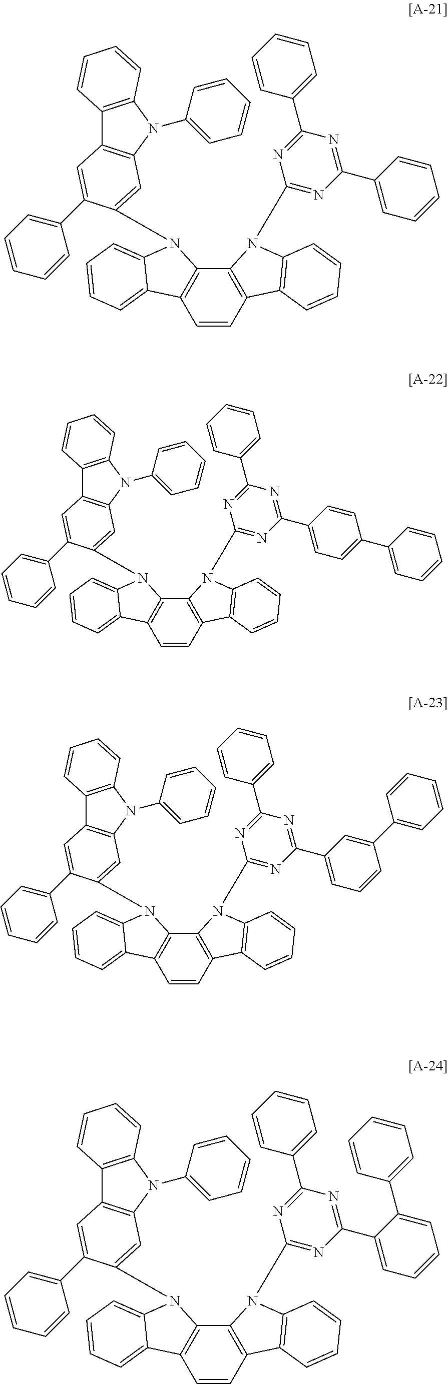

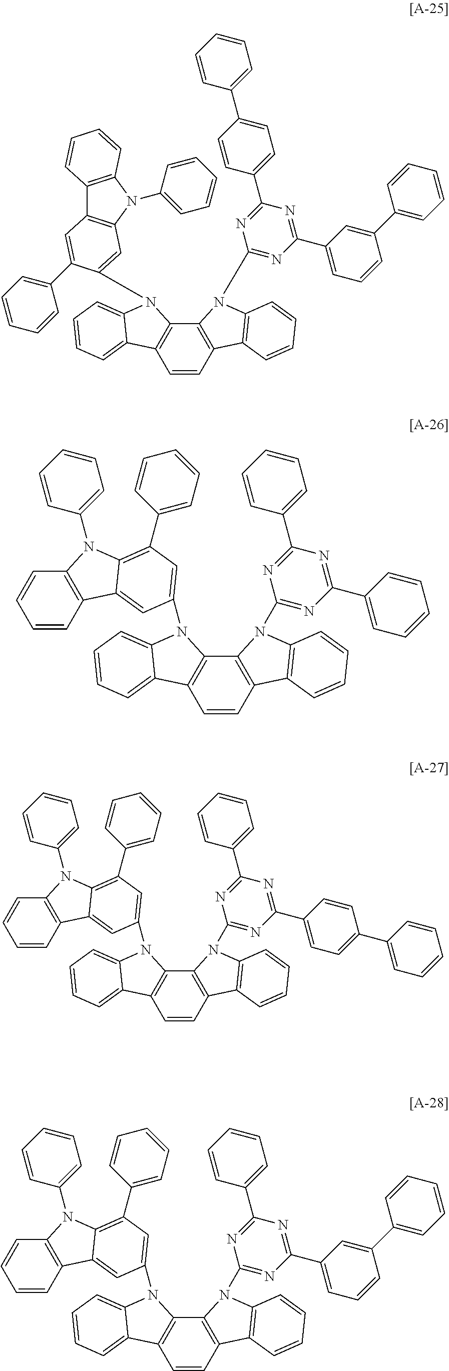 [SMILES: c1ccc(-c2ccc(-c3nc(-c4cccc(-c5ccccc5)c4)nc(-n4c5ccccc5c5ccc6c7ccccc7n(-c7cc8c(cc7-c7ccccc7)c7ccccc7n8-c7ccccc7)c6c54)n3)cc2)cc1 ; c1ccc(-c2nc(-c3ccccc3)nc(-n3c4ccccc4c4ccc5c6ccccc6n(-c6cc(-c7ccccc7)c7c(c6)c6ccccc6n7-c6ccccc6)c5c43)n2)cc1 ; c1ccc(-c2ccc(-c3nc(-c4ccccc4)nc(-n4c5ccccc5c5ccc6c7ccccc7n(-c7cc(-c8ccccc8)c8c(c7)c7ccccc7n8-c7ccccc7)c6c54)n3)cc2)cc1 ; c1ccc(-c2cccc(-c3nc(-c4ccccc4)nc(-n4c5ccccc5c5ccc6c7ccccc7n(-c7cc(-c8ccccc8)c8c(c7)c7ccccc7n8-c7ccccc7)c6c54)n3)c2)cc1]

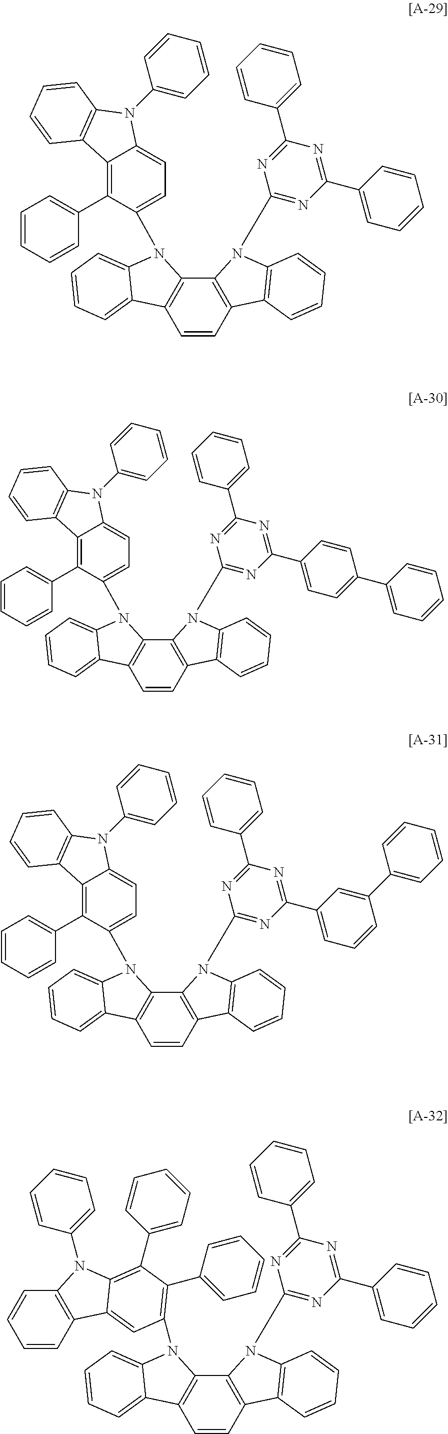 [SMILES: c1ccc(-c2nc(-c3ccccc3)nc(-n3c4ccccc4c4ccc5c6ccccc6n(-c6ccc7c(c6-c6ccccc6)c6ccccc6n7-c6ccccc6)c5c43)n2)cc1 ; c1ccc(-c2ccc(-c3nc(-c4ccccc4)nc(-n4c5ccccc5c5ccc6c7ccccc7n(-c7ccc8c(c7-c7ccccc7)c7ccccc7n8-c7ccccc7)c6c54)n3)cc2)cc1 ; c1ccc(-c2cccc(-c3nc(-c4ccccc4)nc(-n4c5ccccc5c5ccc6c7ccccc7n(-c7ccc8c(c7-c7ccccc7)c7ccccc7n8-c7ccccc7)c6c54)n3)c2)cc1 ; c1ccc(-c2nc(-c3ccccc3)nc(-n3c4ccccc4c4ccc5c6ccccc6n(-c6cc7c8ccccc8n(-c8ccccc8)c7c(-c7ccccc7)c6-c6ccccc6)c5c43)n2)cc1]

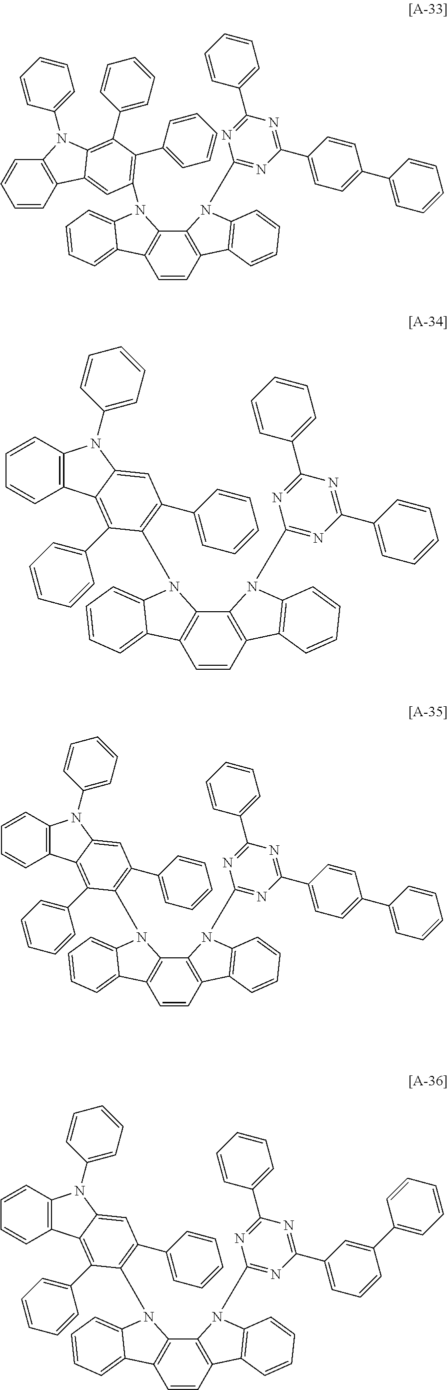

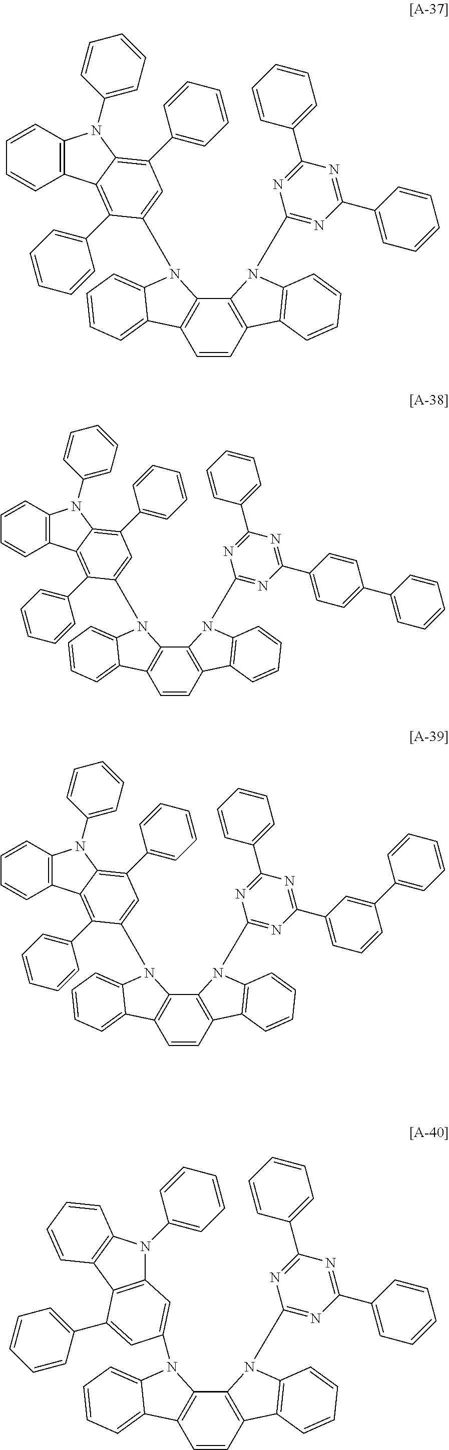 [SMILES: c1ccc(-c2nc(-c3ccccc3)nc(-n3c4ccccc4c4ccc5c6ccccc6n(-c6cc(-c7ccccc7)c7c(c6-c6ccccc6)c6ccccc6n7-c6ccccc6)c5c43)n2)cc1 ; c1ccc(-c2ccc(-c3nc(-c4ccccc4)nc(-n4c5ccccc5c5ccc6c7ccccc7n(-c7cc(-c8ccccc8)c8c(c7-c7ccccc7)c7ccccc7n8-c7ccccc7)c6c54)n3)cc2)cc1 ; c1ccc(-c2cccc(-c3nc(-c4ccccc4)nc(-n4c5ccccc5c5ccc6c7ccccc7n(-c7cc(-c8ccccc8)c8c(c7-c7ccccc7)c7ccccc7n8-c7ccccc7)c6c54)n3)c2)cc1 ; c1ccc(-c2nc(-c3ccccc3)nc(-n3c4ccccc4c4ccc5c6ccccc6n(-c6cc(-c7ccccc7)c7c8ccccc8n(-c8ccccc8)c7c6)c5c43)n2)cc1]

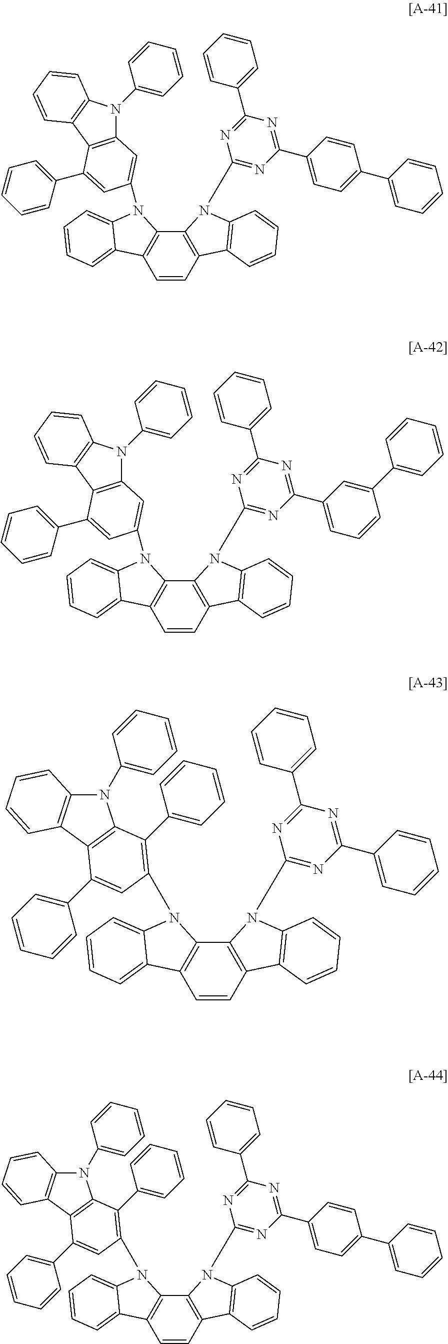 [SMILES: c1ccc(-c2ccc(-c3nc(-c4ccccc4)nc(-n4c5ccccc5c5ccc6c7ccccc7n(-c7cc(-c8ccccc8)c8c9ccccc9n(-c9ccccc9)c8c7)c6c54)n3)cc2)cc1 ; c1ccc(-c2cccc(-c3nc(-c4ccccc4)nc(-n4c5ccccc5c5ccc6c7ccccc7n(-c7cc(-c8ccccc8)c8c9ccccc9n(-c9ccccc9)c8c7)c6c54)n3)c2)cc1 ; c1ccc(-c2nc(-c3ccccc3)nc(-n3c4ccccc4c4ccc5c6ccccc6n(-c6cc(-c7ccccc7)c7c8ccccc8n(-c8ccccc8)c7c6-c6ccccc6)c5c43)n2)cc1 ; c1ccc(-c2ccc(-c3nc(-c4ccccc4)nc(-n4c5ccccc5c5ccc6c7ccccc7n(-c7cc(-c8ccccc8)c8c9ccccc9n(-c9ccccc9)c8c7-c7ccccc7)c6c54)n3)cc2)cc1]

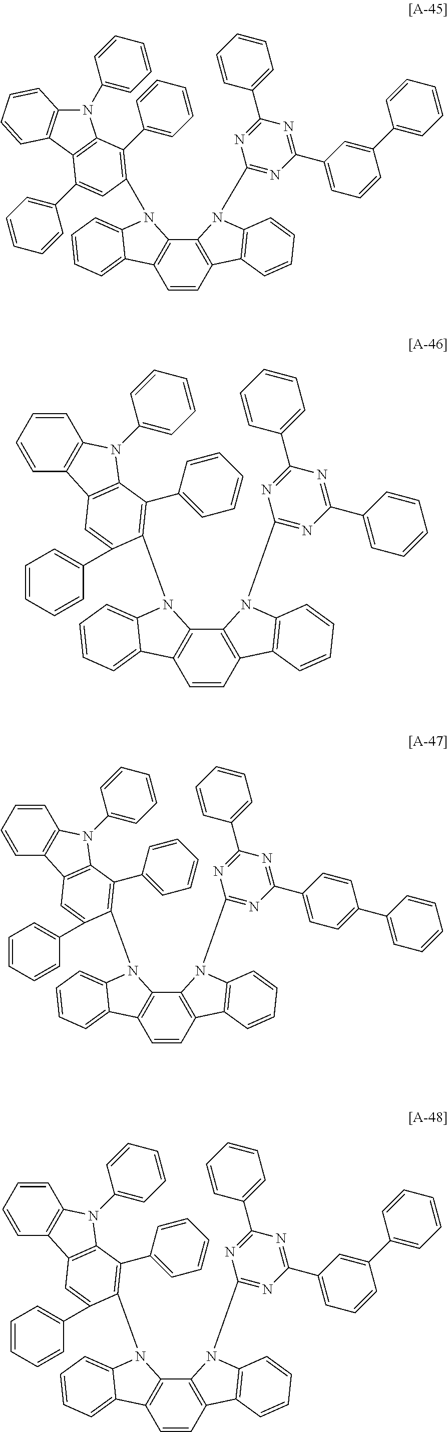

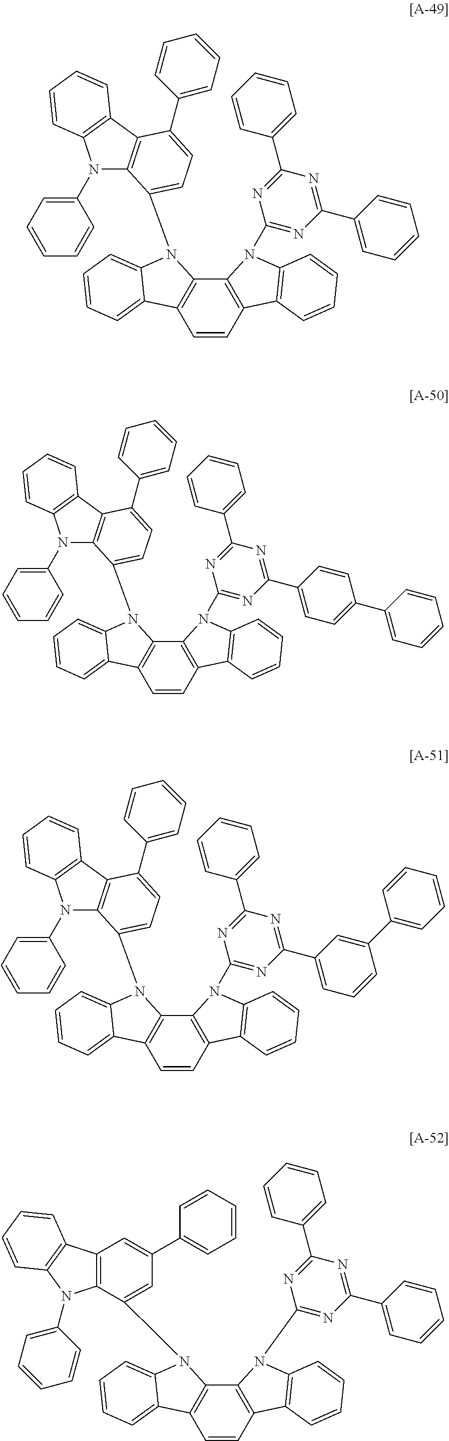 [SMILES: c1ccc(-c2nc(-c3ccccc3)nc(-n3c4ccccc4c4ccc5c6ccccc6n(-c6ccc(-c7ccccc7)c7c8ccccc8n(-c8ccccc8)c67)c5c43)n2)cc1 ; c1ccc(-c2ccc(-c3nc(-c4ccccc4)nc(-n4c5ccccc5c5ccc6c7ccccc7n(-c7ccc(-c8ccccc8)c8c9ccccc9n(-c9ccccc9)c78)c6c54)n3)cc2)cc1 ; c1ccc(-c2cccc(-c3nc(-c4ccccc4)nc(-n4c5ccccc5c5ccc6c7ccccc7n(-c7ccc(-c8ccccc8)c8c9ccccc9n(-c9ccccc9)c78)c6c54)n3)c2)cc1 ; c1ccc(-c2cc(-n3c4ccccc4c4ccc5c6ccccc6n(-c6nc(-c7ccccc7)nc(-c7ccccc7)n6)c5c43)c3c(c2)c2ccccc2n3-c2ccccc2)cc1]

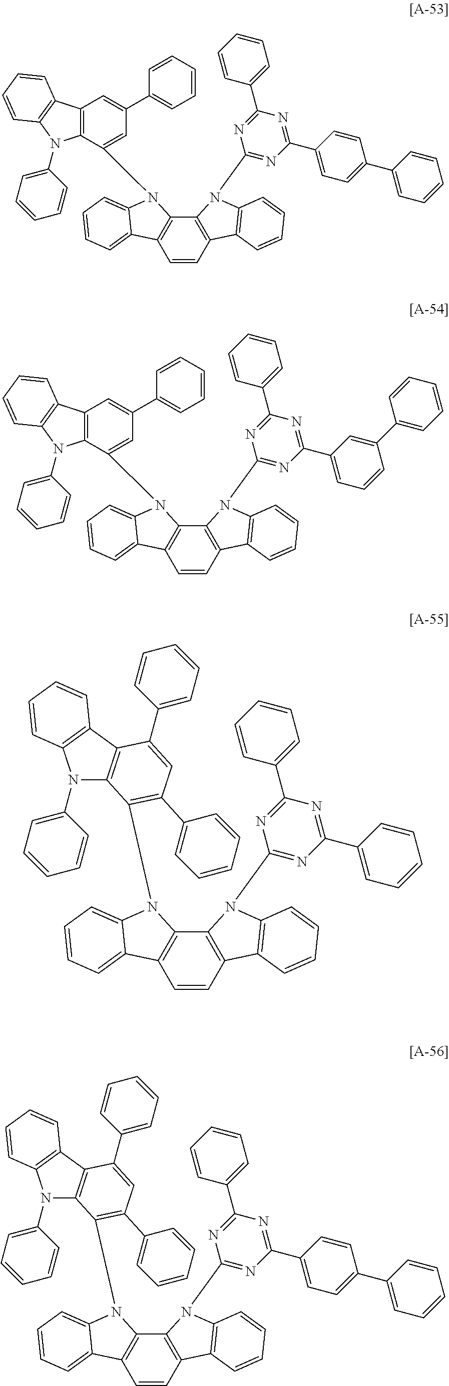 [SMILES: c1ccc(-c2ccc(-c3nc(-c4ccccc4)nc(-n4c5ccccc5c5ccc6c7ccccc7n(-c7cc(-c8ccccc8)cc8c9ccccc9n(-c9ccccc9)c78)c6c54)n3)cc2)cc1 ; c1ccc(-c2cccc(-c3nc(-c4ccccc4)nc(-n4c5ccccc5c5ccc6c7ccccc7n(-c7cc(-c8ccccc8)cc8c9ccccc9n(-c9ccccc9)c78)c6c54)n3)c2)cc1 ; c1ccc(-c2nc(-c3ccccc3)nc(-n3c4ccccc4c4ccc5c6ccccc6n(-c6c(-c7ccccc7)cc(-c7ccccc7)c7c8ccccc8n(-c8ccccc8)c67)c5c43)n2)cc1 ; c1ccc(-c2ccc(-c3nc(-c4ccccc4)nc(-n4c5ccccc5c5ccc6c7ccccc7n(-c7c(-c8ccccc8)cc(-c8ccccc8)c8c9ccccc9n(-c9ccccc9)c78)c6c54)n3)cc2)cc1]

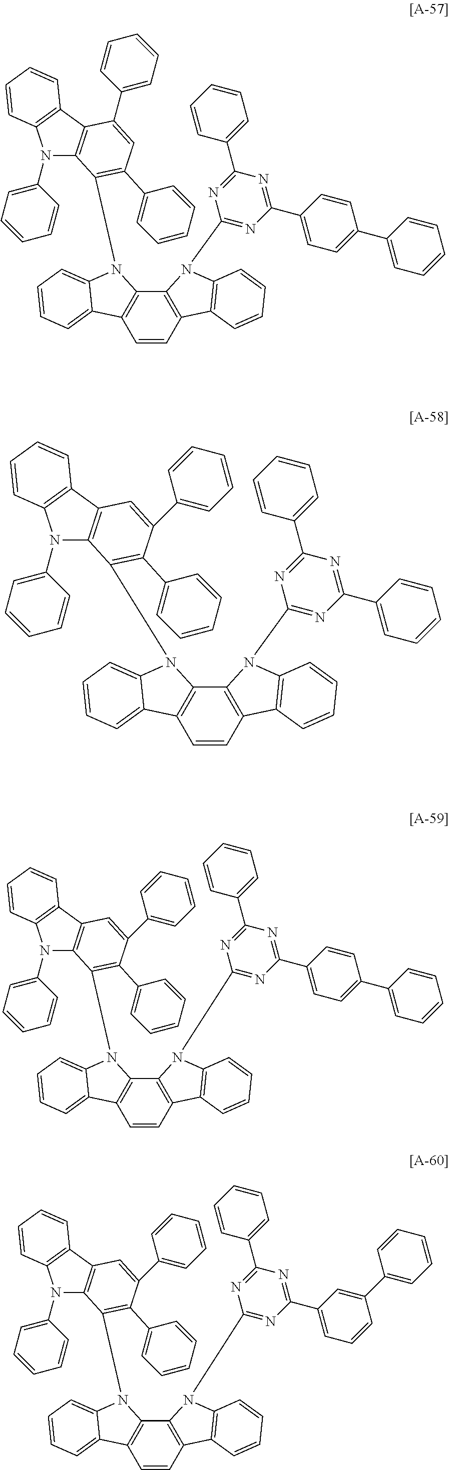

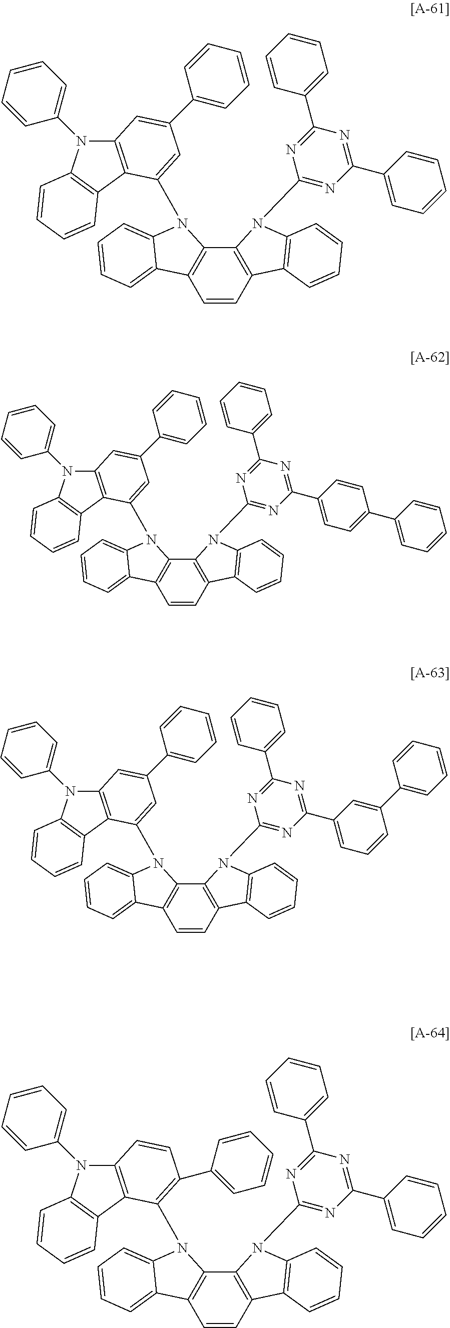 [SMILES: c1ccc(-c2cc(-n3c4ccccc4c4ccc5c6ccccc6n(-c6nc(-c7ccccc7)nc(-c7ccccc7)n6)c5c43)c3c4ccccc4n(-c4ccccc4)c3c2)cc1 ; c1ccc(-c2ccc(-c3nc(-c4ccccc4)nc(-n4c5ccccc5c5ccc6c7ccccc7n(-c7cc(-c8ccccc8)cc8c7c7ccccc7n8-c7ccccc7)c6c54)n3)cc2)cc1 ; c1ccc(-c2cccc(-c3nc(-c4ccccc4)nc(-n4c5ccccc5c5ccc6c7ccccc7n(-c7cc(-c8ccccc8)cc8c7c7ccccc7n8-c7ccccc7)c6c54)n3)c2)cc1 ; c1ccc(-c2nc(-c3ccccc3)nc(-n3c4ccccc4c4ccc5c6ccccc6n(-c6c(-c7ccccc7)ccc7c6c6ccccc6n7-c6ccccc6)c5c43)n2)cc1]

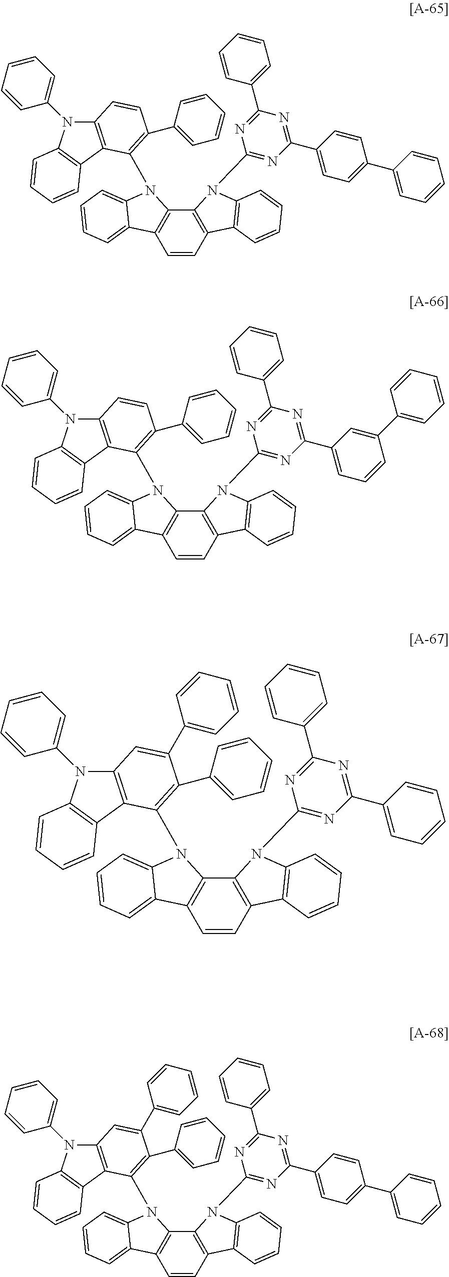 [SMILES: c1ccc(-c2ccc(-c3nc(-c4ccccc4)nc(-n4c5ccccc5c5ccc6c7ccccc7n(-c7c(-c8ccccc8)ccc8c7c7ccccc7n8-c7ccccc7)c6c54)n3)cc2)cc1 ; c1ccc(-c2cccc(-c3nc(-c4ccccc4)nc(-n4c5ccccc5c5ccc6c7ccccc7n(-c7c(-c8ccccc8)ccc8c7c7ccccc7n8-c7ccccc7)c6c54)n3)c2)cc1 ; c1ccc(-c2nc(-c3ccccc3)nc(-n3c4ccccc4c4ccc5c6ccccc6n(-c6c(-c7ccccc7)c(-c7ccccc7)cc7c6c6ccccc6n7-c6ccccc6)c5c43)n2)cc1 ; c1ccc(-c2ccc(-c3nc(-c4ccccc4)nc(-n4c5ccccc5c5ccc6c7ccccc7n(-c7c(-c8ccccc8)c(-c8ccccc8)cc8c7c7ccccc7n8-c7ccccc7)c6c54)n3)cc2)cc1]

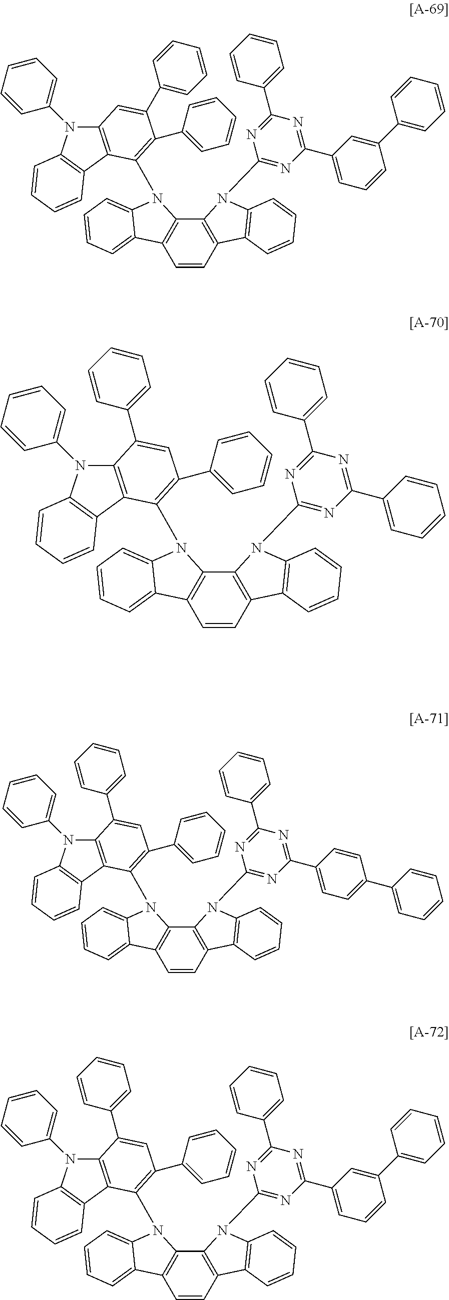

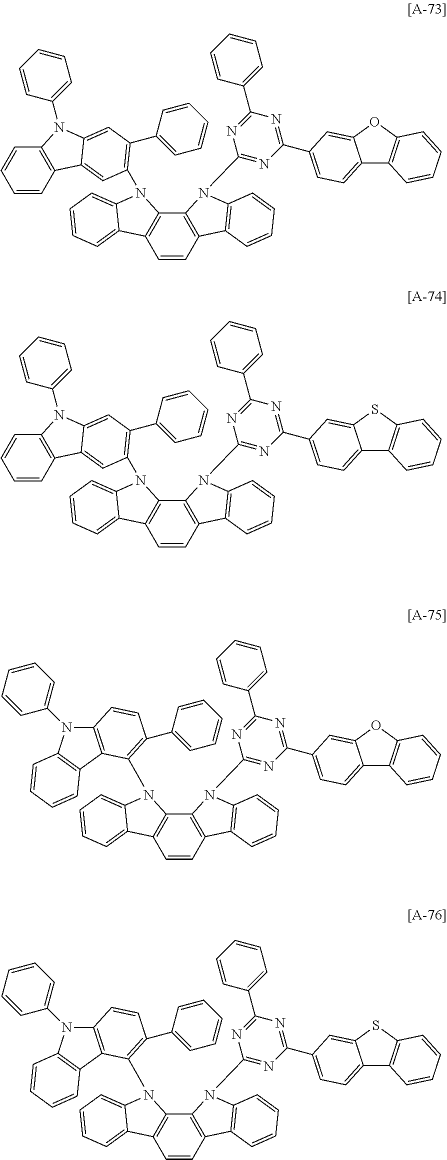 [SMILES: c1ccc(-c2nc(-c3ccc4c(c3)oc3ccccc34)nc(-n3c4ccccc4c4ccc5c6ccccc6n(-c6cc7c8ccccc8n(-c8ccccc8)c7cc6-c6ccccc6)c5c43)n2)cc1 ; c1ccc(-c2nc(-c3ccc4c(c3)sc3ccccc34)nc(-n3c4ccccc4c4ccc5c6ccccc6n(-c6cc7c8ccccc8n(-c8ccccc8)c7cc6-c6ccccc6)c5c43)n2)cc1 ; c1ccc(-c2nc(-c3ccc4c(c3)oc3ccccc34)nc(-n3c4ccccc4c4ccc5c6ccccc6n(-c6c(-c7ccccc7)ccc7c6c6ccccc6n7-c6ccccc6)c5c43)n2)cc1 ; c1ccc(-c2nc(-c3ccc4c(c3)sc3ccccc34)nc(-n3c4ccccc4c4ccc5c6ccccc6n(-c6c(-c7ccccc7)ccc7c6c6ccccc6n7-c6ccccc6)c5c43)n2)cc1]

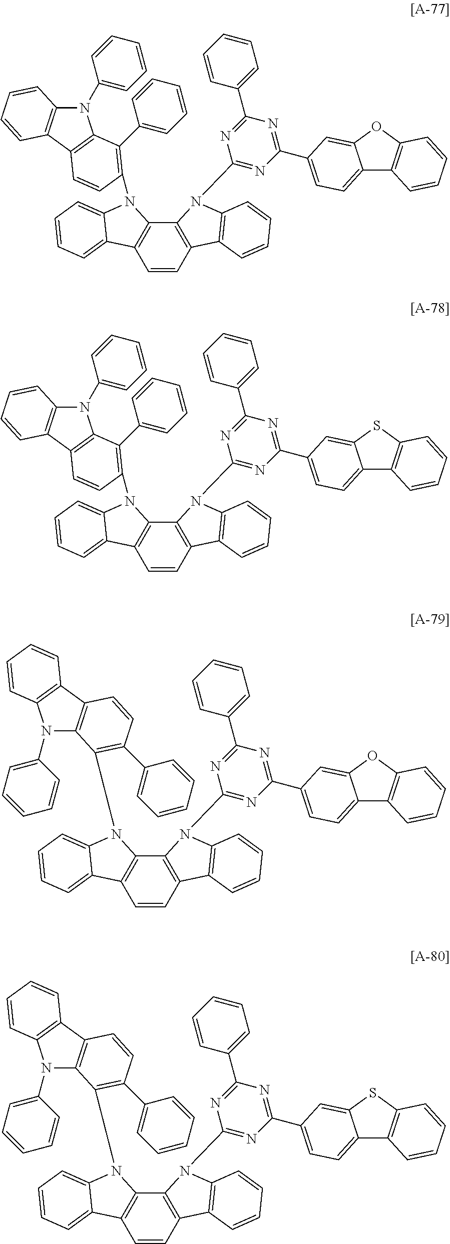 [SMILES: c1ccc(-c2nc(-c3ccc4c(c3)oc3ccccc34)nc(-n3c4ccccc4c4ccc5c6ccccc6n(-c6ccc7c8ccccc8n(-c8ccccc8)c7c6-c6ccccc6)c5c43)n2)cc1 ; c1ccc(-c2nc(-c3ccc4c(c3)sc3ccccc34)nc(-n3c4ccccc4c4ccc5c6ccccc6n(-c6ccc7c8ccccc8n(-c8ccccc8)c7c6-c6ccccc6)c5c43)n2)cc1 ; c1ccc(-c2nc(-c3ccc4c(c3)oc3ccccc34)nc(-n3c4ccccc4c4ccc5c6ccccc6n(-c6c(-c7ccccc7)ccc7c8ccccc8n(-c8ccccc8)c67)c5c43)n2)cc1 ; c1ccc(-c2nc(-c3ccc4c(c3)sc3ccccc34)nc(-n3c4ccccc4c4ccc5c6ccccc6n(-c6c(-c7ccccc7)ccc7c8ccccc8n(-c8ccccc8)c67)c5c43)n2)cc1]

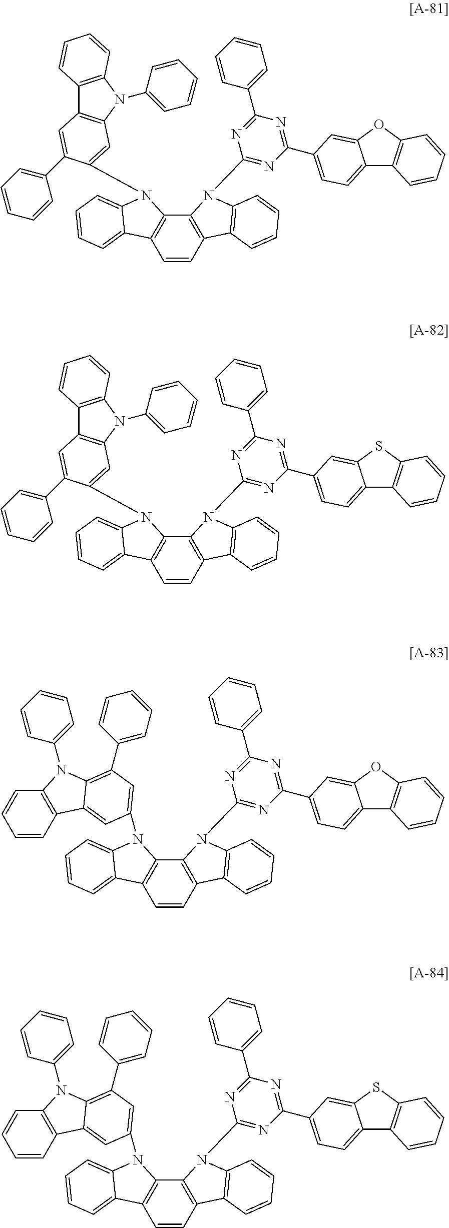 [SMILES: c1ccc(-c2nc(-c3ccc4c(c3)oc3ccccc34)nc(-n3c4ccccc4c4ccc5c6ccccc6n(-c6cc7c(cc6-c6ccccc6)c6ccccc6n7-c6ccccc6)c5c43)n2)cc1 ; c1ccc(-c2nc(-c3ccc4c(c3)sc3ccccc34)nc(-n3c4ccccc4c4ccc5c6ccccc6n(-c6cc7c(cc6-c6ccccc6)c6ccccc6n7-c6ccccc6)c5c43)n2)cc1 ; c1ccc(-c2nc(-c3ccc4c(c3)oc3ccccc34)nc(-n3c4ccccc4c4ccc5c6ccccc6n(-c6cc(-c7ccccc7)c7c(c6)c6ccccc6n7-c6ccccc6)c5c43)n2)cc1 ; c1ccc(-c2nc(-c3ccc4c(c3)sc3ccccc34)nc(-n3c4ccccc4c4ccc5c6ccccc6n(-c6cc(-c7ccccc7)c7c(c6)c6ccccc6n7-c6ccccc6)c5c43)n2)cc1]

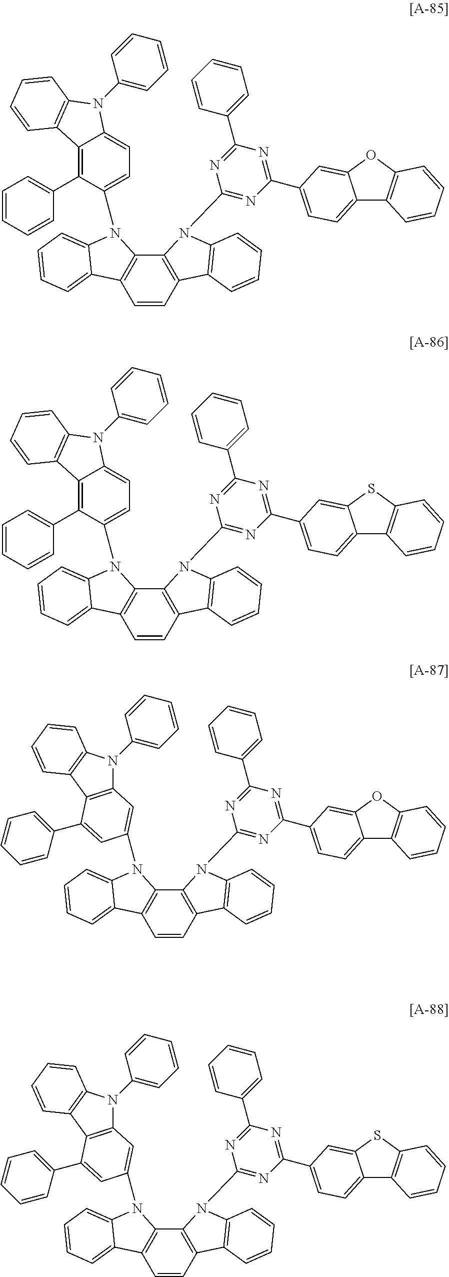 [SMILES: c1ccc(-c2nc(-c3ccc4c(c3)oc3ccccc34)nc(-n3c4ccccc4c4ccc5c6ccccc6n(-c6ccc7c(c6-c6ccccc6)c6ccccc6n7-c6ccccc6)c5c43)n2)cc1 ; c1ccc(-c2nc(-c3ccc4c(c3)sc3ccccc34)nc(-n3c4ccccc4c4ccc5c6ccccc6n(-c6ccc7c(c6-c6ccccc6)c6ccccc6n7-c6ccccc6)c5c43)n2)cc1 ; c1ccc(-c2nc(-c3ccc4c(c3)oc3ccccc34)nc(-n3c4ccccc4c4ccc5c6ccccc6n(-c6cc(-c7ccccc7)c7c8ccccc8n(-c8ccccc8)c7c6)c5c43)n2)cc1 ; c1ccc(-c2nc(-c3ccc4c(c3)sc3ccccc34)nc(-n3c4ccccc4c4ccc5c6ccccc6n(-c6cc(-c7ccccc7)c7c8ccccc8n(-c8ccccc8)c7c6)c5c43)n2)cc1]

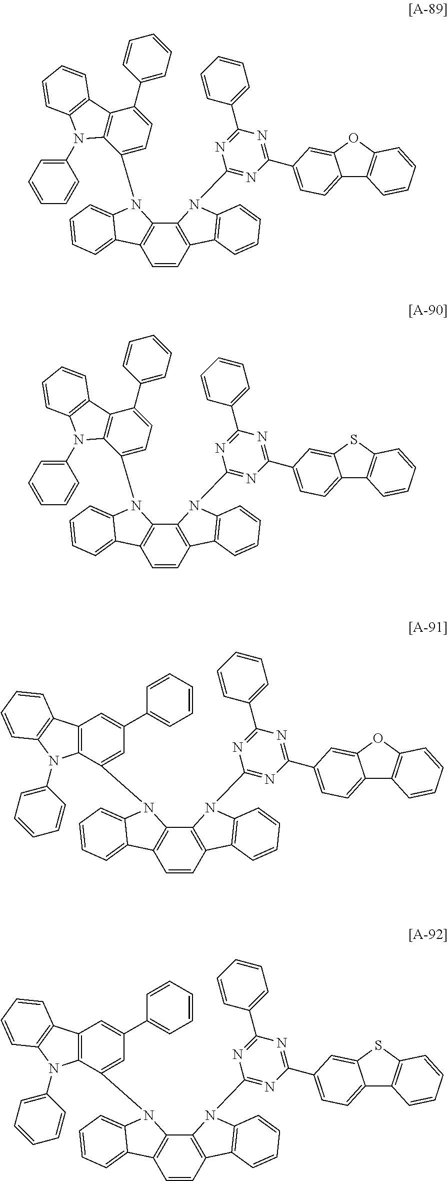 [SMILES: c1ccc(-c2nc(-c3ccc4c(c3)oc3ccccc34)nc(-n3c4ccccc4c4ccc5c6ccccc6n(-c6ccc(-c7ccccc7)c7c8ccccc8n(-c8ccccc8)c67)c5c43)n2)cc1 ; c1ccc(-c2nc(-c3ccc4c(c3)sc3ccccc34)nc(-n3c4ccccc4c4ccc5c6ccccc6n(-c6ccc(-c7ccccc7)c7c8ccccc8n(-c8ccccc8)c67)c5c43)n2)cc1 ; c1ccc(-c2cc(-n3c4ccccc4c4ccc5c6ccccc6n(-c6nc(-c7ccccc7)nc(-c7ccc8c(c7)oc7ccccc78)n6)c5c43)c3c(c2)c2ccccc2n3-c2ccccc2)cc1 ; c1ccc(-c2cc(-n3c4ccccc4c4ccc5c6ccccc6n(-c6nc(-c7ccccc7)nc(-c7ccc8c(c7)sc7ccccc78)n6)c5c43)c3c(c2)c2ccccc2n3-c2ccccc2)cc1]

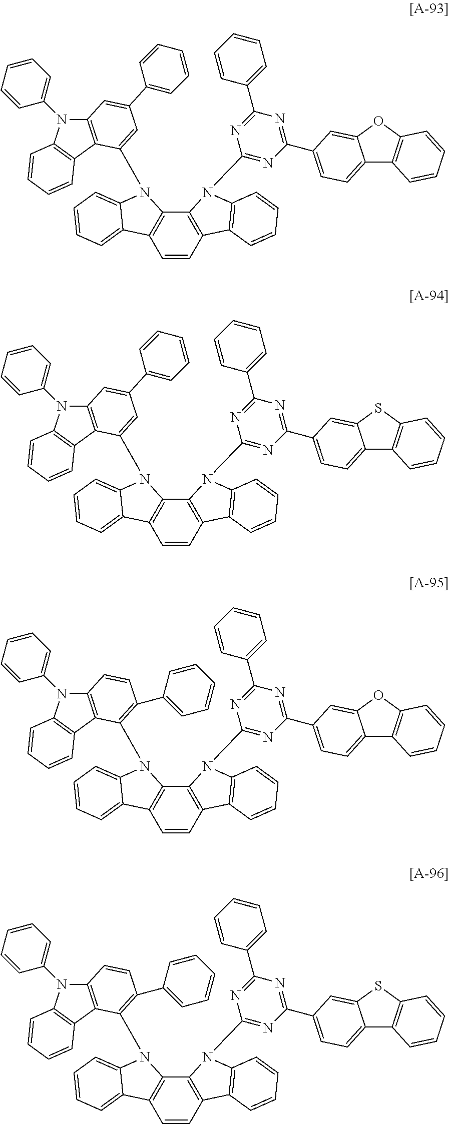 [SMILES: c1ccc(-c2cc(-n3c4ccccc4c4ccc5c6ccccc6n(-c6nc(-c7ccccc7)nc(-c7ccc8c(c7)oc7ccccc78)n6)c5c43)c3c4ccccc4n(-c4ccccc4)c3c2)cc1 ; c1ccc(-c2cc(-n3c4ccccc4c4ccc5c6ccccc6n(-c6nc(-c7ccccc7)nc(-c7ccc8c(c7)sc7ccccc78)n6)c5c43)c3c4ccccc4n(-c4ccccc4)c3c2)cc1 ; c1ccc(-c2nc(-c3ccc4c(c3)oc3ccccc34)nc(-n3c4ccccc4c4ccc5c6ccccc6n(-c6c(-c7ccccc7)ccc7c6c6ccccc6n7-c6ccccc6)c5c43)n2)cc1 ; c1ccc(-c2nc(-c3ccc4c(c3)sc3ccccc34)nc(-n3c4ccccc4c4ccc5c6ccccc6n(-c6c(-c7ccccc7)ccc7c6c6ccccc6n7-c6ccccc6)c5c43)n2)cc1]

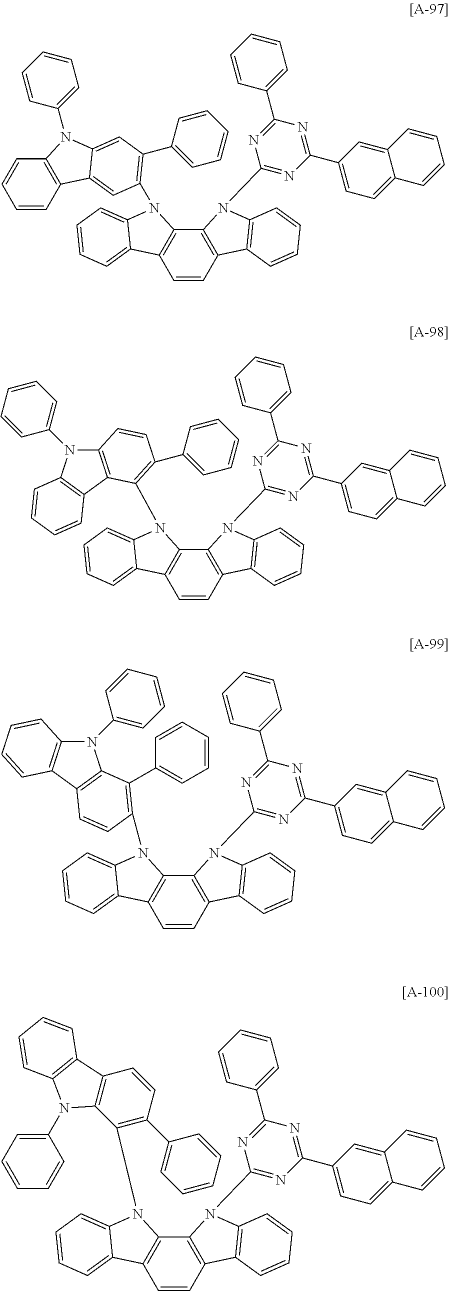 [SMILES: c1ccc(-c2nc(-c3ccc4ccccc4c3)nc(-n3c4ccccc4c4ccc5c6ccccc6n(-c6cc7c8ccccc8n(-c8ccccc8)c7cc6-c6ccccc6)c5c43)n2)cc1 ; c1ccc(-c2nc(-c3ccc4ccccc4c3)nc(-n3c4ccccc4c4ccc5c6ccccc6n(-c6c(-c7ccccc7)ccc7c6c6ccccc6n7-c6ccccc6)c5c43)n2)cc1 ; c1ccc(-c2nc(-c3ccc4ccccc4c3)nc(-n3c4ccccc4c4ccc5c6ccccc6n(-c6ccc7c8ccccc8n(-c8ccccc8)c7c6-c6ccccc6)c5c43)n2)cc1 ; c1ccc(-c2nc(-c3ccc4ccccc4c3)nc(-n3c4ccccc4c4ccc5c6ccccc6n(-c6c(-c7ccccc7)ccc7c8ccccc8n(-c8ccccc8)c67)c5c43)n2)cc1]

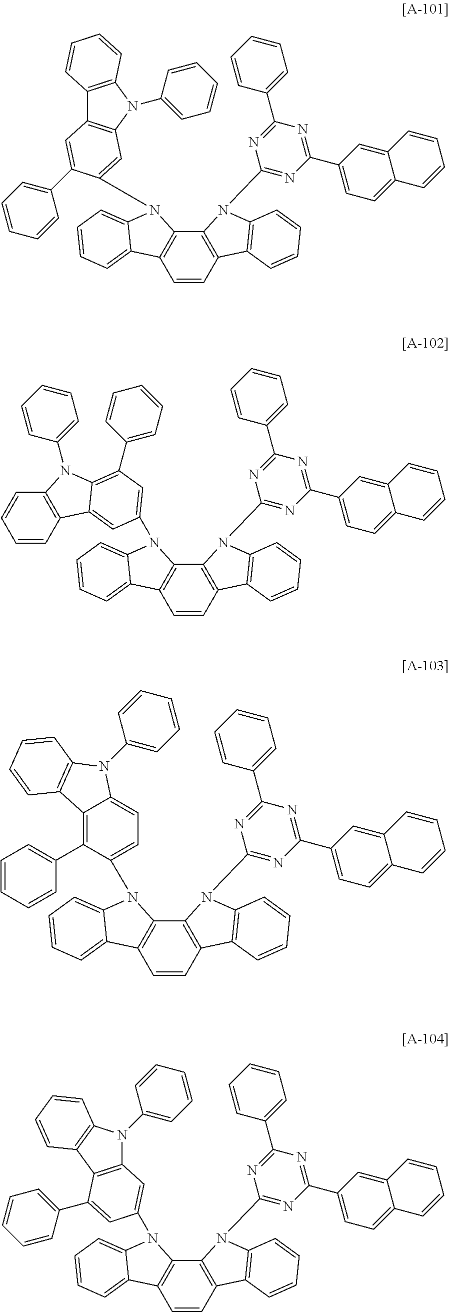 [SMILES: c1ccc(-c2nc(-c3ccc4ccccc4c3)nc(-n3c4ccccc4c4ccc5c6ccccc6n(-c6cc7c(cc6-c6ccccc6)c6ccccc6n7-c6ccccc6)c5c43)n2)cc1 ; c1ccc(-c2nc(-c3ccc4ccccc4c3)nc(-n3c4ccccc4c4ccc5c6ccccc6n(-c6cc(-c7ccccc7)c7c(c6)c6ccccc6n7-c6ccccc6)c5c43)n2)cc1 ; c1ccc(-c2nc(-c3ccc4ccccc4c3)nc(-n3c4ccccc4c4ccc5c6ccccc6n(-c6ccc7c(c6-c6ccccc6)c6ccccc6n7-c6ccccc6)c5c43)n2)cc1 ; c1ccc(-c2nc(-c3ccc4ccccc4c3)nc(-n3c4ccccc4c4ccc5c6ccccc6n(-c6cc(-c7ccccc7)c7c8ccccc8n(-c8ccccc8)c7c6)c5c43)n2)cc1]

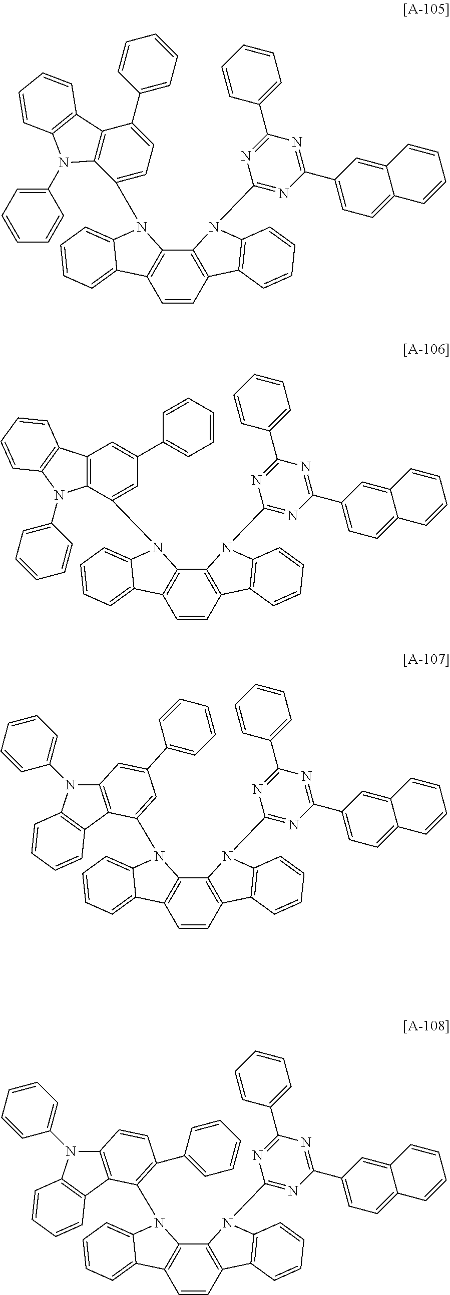 [SMILES: c1ccc(-c2nc(-c3ccc4ccccc4c3)nc(-n3c4ccccc4c4ccc5c6ccccc6n(-c6ccc(-c7ccccc7)c7c8ccccc8n(-c8ccccc8)c67)c5c43)n2)cc1 ; c1ccc(-c2cc(-n3c4ccccc4c4ccc5c6ccccc6n(-c6nc(-c7ccccc7)nc(-c7ccc8ccccc8c7)n6)c5c43)c3c(c2)c2ccccc2n3-c2ccccc2)cc1 ; c1ccc(-c2cc(-n3c4ccccc4c4ccc5c6ccccc6n(-c6nc(-c7ccccc7)nc(-c7ccc8ccccc8c7)n6)c5c43)c3c4ccccc4n(-c4ccccc4)c3c2)cc1 ; c1ccc(-c2nc(-c3ccc4ccccc4c3)nc(-n3c4ccccc4c4ccc5c6ccccc6n(-c6c(-c7ccccc7)ccc7c6c6ccccc6n7-c6ccccc6)c5c43)n2)cc1]

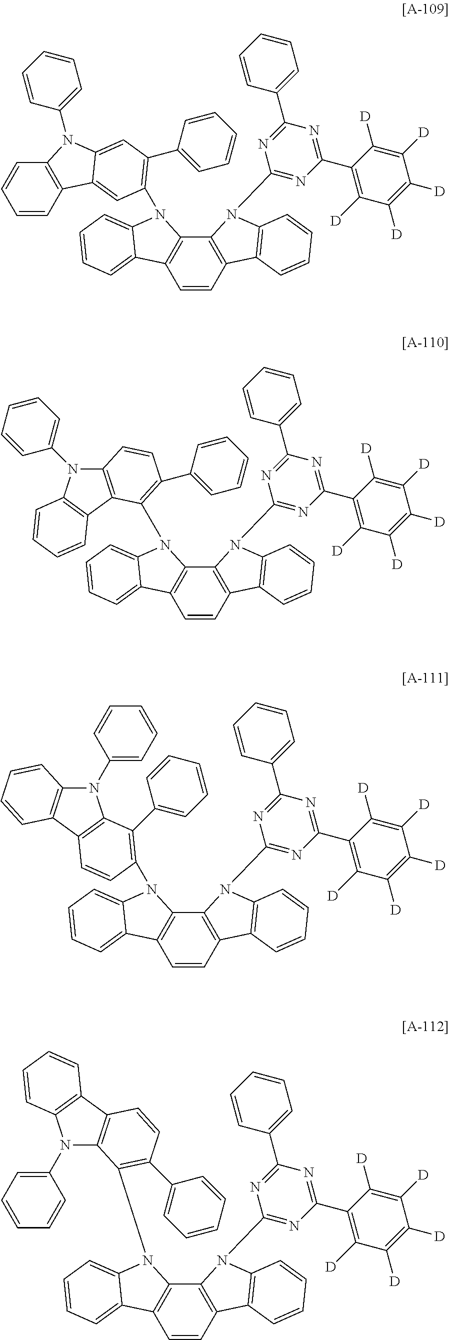

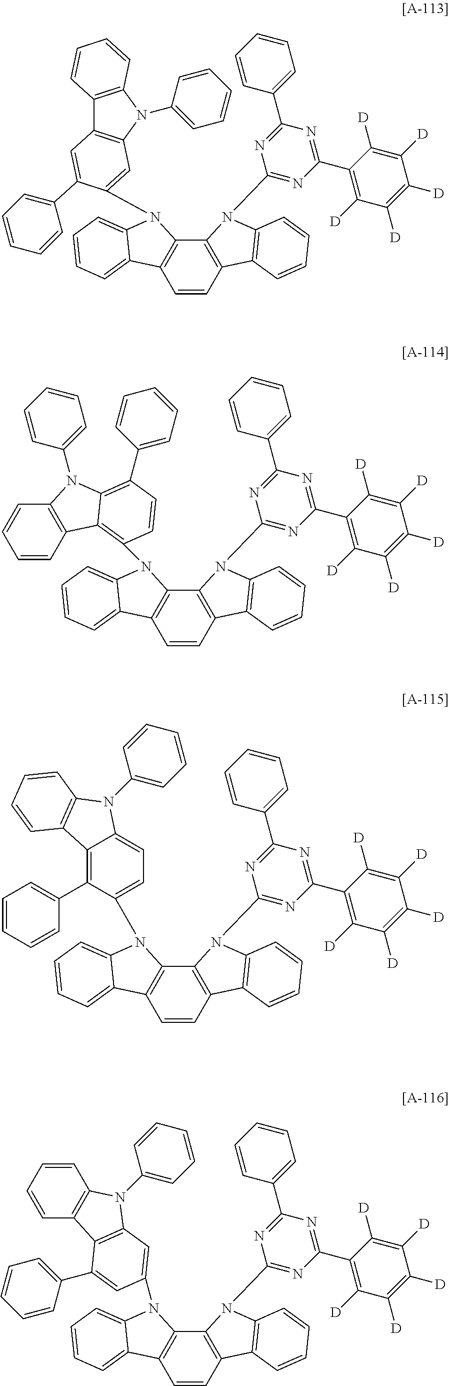

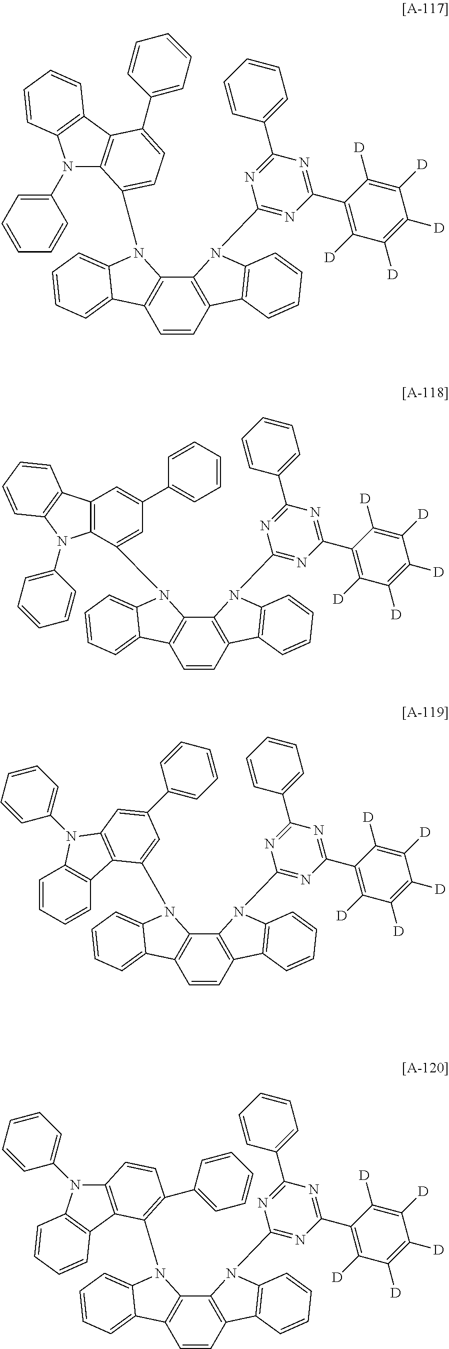

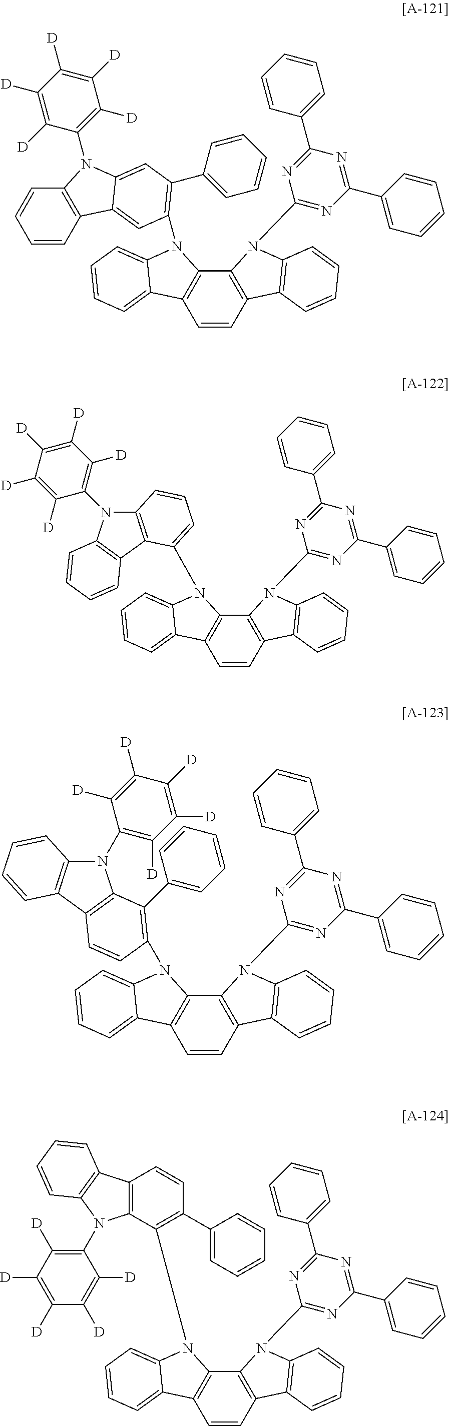

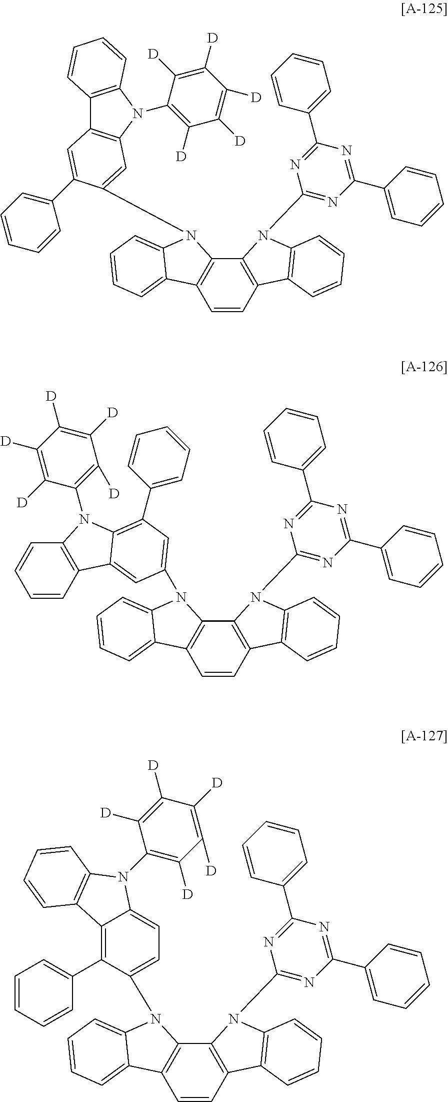 [SMILES: [2H]c1c([2H])c([2H])c(-n2c3ccccc3c3cc(-c4ccccc4)c(-n4c5ccccc5c5ccc6c7ccccc7n(-c7nc(-c8ccccc8)nc(-c8ccccc8)n7)c6c54)cc32)c([2H])c1[2H] ; [2H]c1c([2H])c([2H])c(-n2c3ccccc3c3cc(-n4c5ccccc5c5ccc6c7ccccc7n(-c7nc(-c8ccccc8)nc(-c8ccccc8)n7)c6c54)cc(-c4ccccc4)c32)c([2H])c1[2H] ; [2H]c1c([2H])c([2H])c(-n2c3ccccc3c3c(-c4ccccc4)c(-n4c5ccccc5c5ccc6c7ccccc7n(-c7nc(-c8ccccc8)nc(-c8ccccc8)n7)c6c54)ccc32)c([2H])c1[2H]]

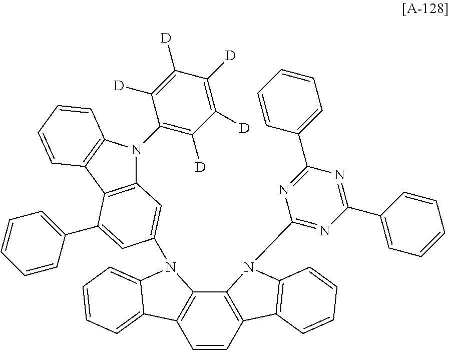 [SMILES: [2H]c1c([2H])c([2H])c(-n2c3ccccc3c3c(-c4ccccc4)cc(-n4c5ccccc5c5ccc6c7ccccc7n(-c7nc(-c8ccccc8)nc(-c8ccccc8)n7)c6c54)cc32)c([2H])c1[2H]]

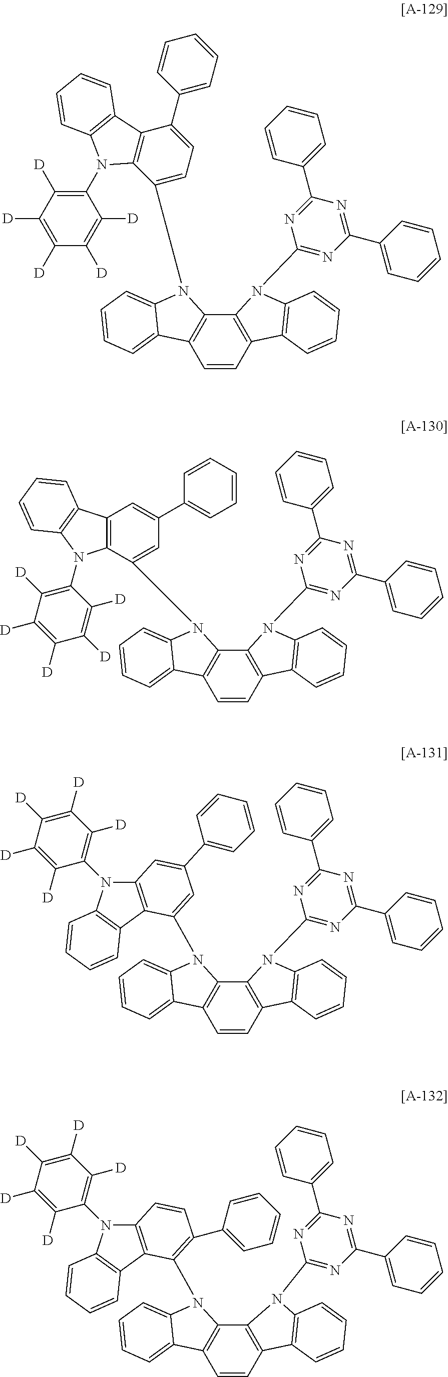

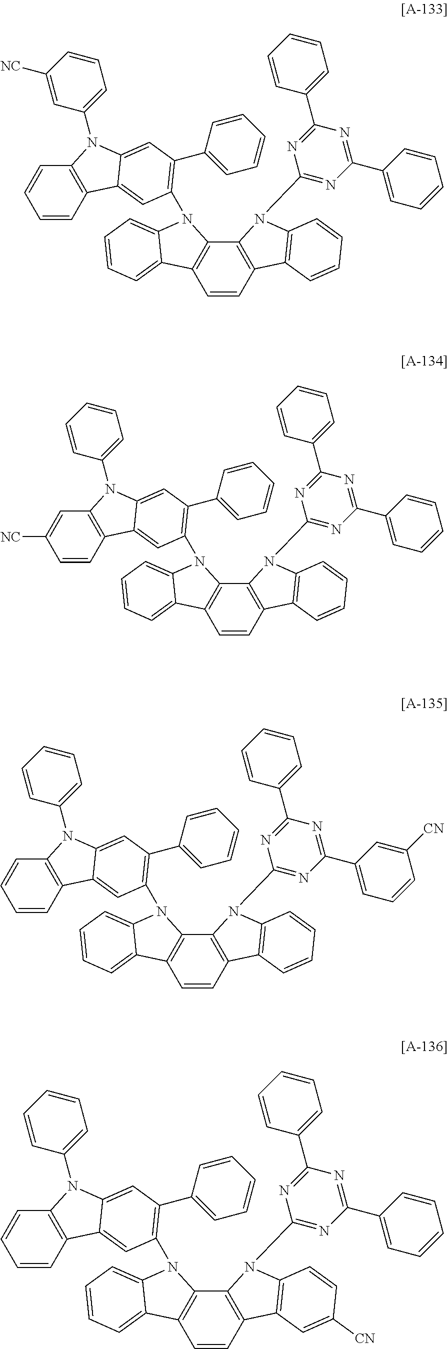 [SMILES: N#Cc1cccc(-n2c3ccccc3c3cc(-n4c5ccccc5c5ccc6c7ccccc7n(-c7nc(-c8ccccc8)nc(-c8ccccc8)n7)c6c54)c(-c4ccccc4)cc32)c1 ; N#Cc1ccc2c3cc(-n4c5ccccc5c5ccc6c7ccccc7n(-c7nc(-c8ccccc8)nc(-c8ccccc8)n7)c6c54)c(-c4ccccc4)cc3n(-c3ccccc3)c2c1 ; N#Cc1cccc(-c2nc(-c3ccccc3)nc(-n3c4ccccc4c4ccc5c6ccccc6n(-c6cc7c8ccccc8n(-c8ccccc8)c7cc6-c6ccccc6)c5c43)n2)c1 ; N#Cc1ccc2c(c1)c1ccc3c4ccccc4n(-c4cc5c6ccccc6n(-c6ccccc6)c5cc4-c4ccccc4)c3c1n2-c1nc(-c2ccccc2)nc(-c2ccccc2)n1]

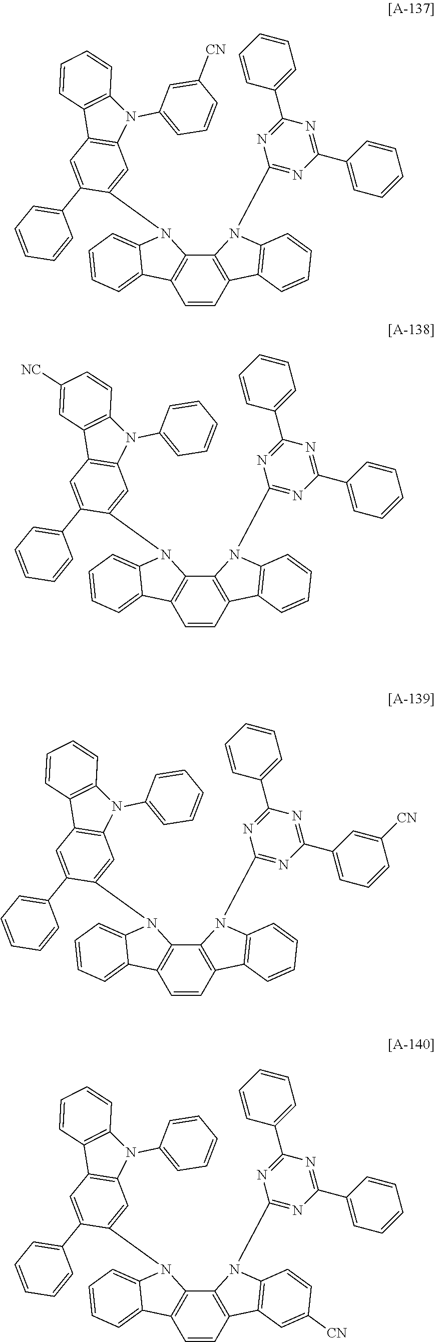 [SMILES: N#Cc1cccc(-n2c3ccccc3c3cc(-c4ccccc4)c(-n4c5ccccc5c5ccc6c7ccccc7n(-c7nc(-c8ccccc8)nc(-c8ccccc8)n7)c6c54)cc32)c1 ; N#Cc1ccc2c(c1)c1cc(-c3ccccc3)c(-n3c4ccccc4c4ccc5c6ccccc6n(-c6nc(-c7ccccc7)nc(-c7ccccc7)n6)c5c43)cc1n2-c1ccccc1 ; N#Cc1cccc(-c2nc(-c3ccccc3)nc(-n3c4ccccc4c4ccc5c6ccccc6n(-c6cc7c(cc6-c6ccccc6)c6ccccc6n7-c6ccccc6)c5c43)n2)c1 ; N#Cc1ccc2c(c1)c1ccc3c4ccccc4n(-c4cc5c(cc4-c4ccccc4)c4ccccc4n5-c4ccccc4)c3c1n2-c1nc(-c2ccccc2)nc(-c2ccccc2)n1]

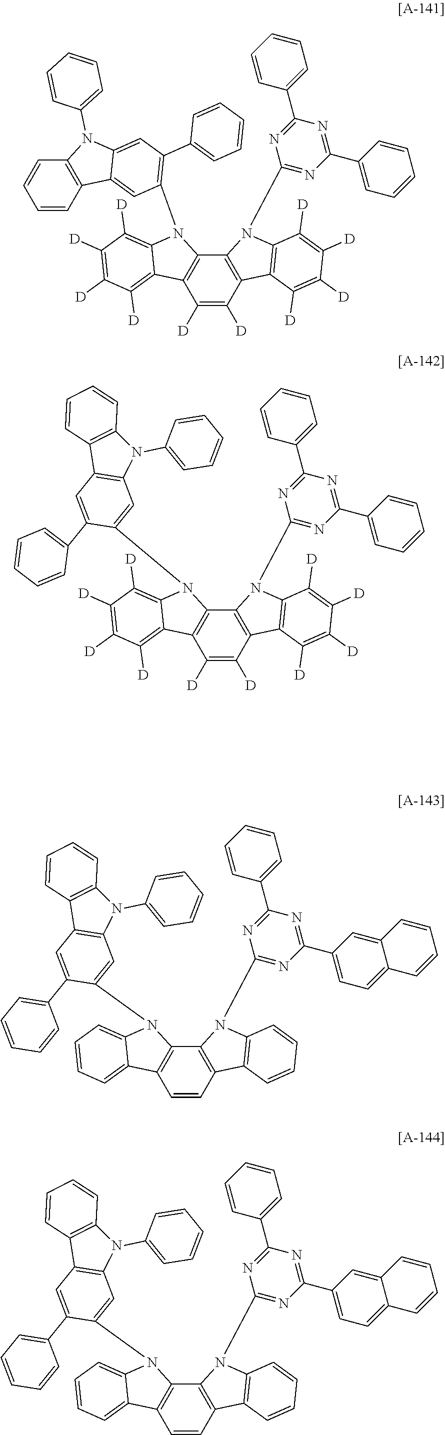

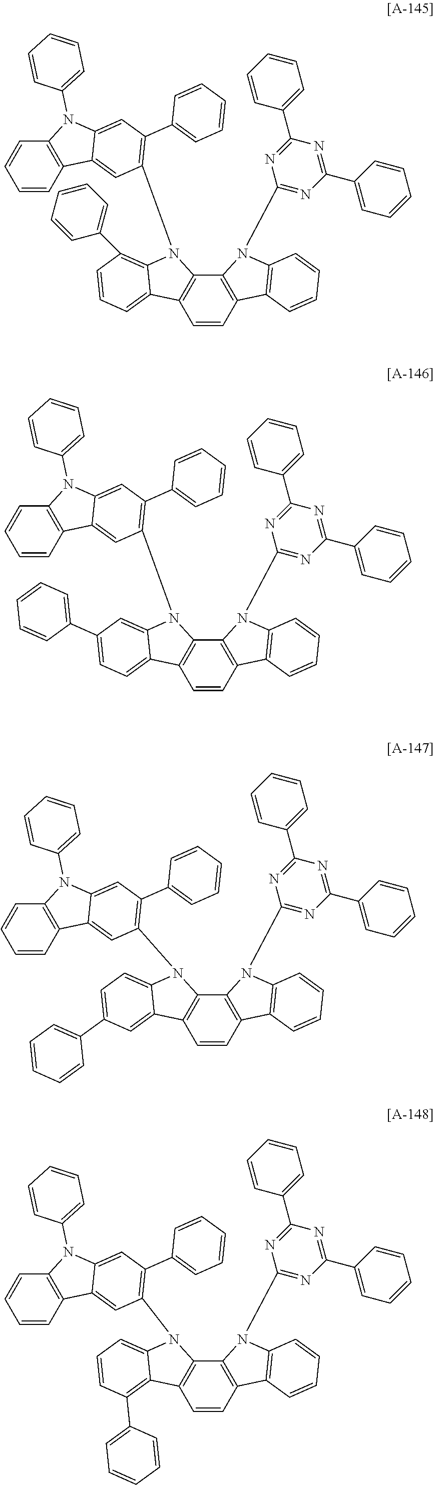 [SMILES: c1ccc(-c2nc(-c3ccccc3)nc(-n3c4ccccc4c4ccc5c6cccc(-c7ccccc7)c6n(-c6cc7c8ccccc8n(-c8ccccc8)c7cc6-c6ccccc6)c5c43)n2)cc1 ; c1ccc(-c2ccc3c4ccc5c6ccccc6n(-c6nc(-c7ccccc7)nc(-c7ccccc7)n6)c5c4n(-c4cc5c6ccccc6n(-c6ccccc6)c5cc4-c4ccccc4)c3c2)cc1 ; c1ccc(-c2ccc3c(c2)c2ccc4c5ccccc5n(-c5nc(-c6ccccc6)nc(-c6ccccc6)n5)c4c2n3-c2cc3c4ccccc4n(-c4ccccc4)c3cc2-c2ccccc2)cc1 ; c1ccc(-c2nc(-c3ccccc3)nc(-n3c4ccccc4c4ccc5c6c(-c7ccccc7)cccc6n(-c6cc7c8ccccc8n(-c8ccccc8)c7cc6-c6ccccc6)c5c43)n2)cc1]

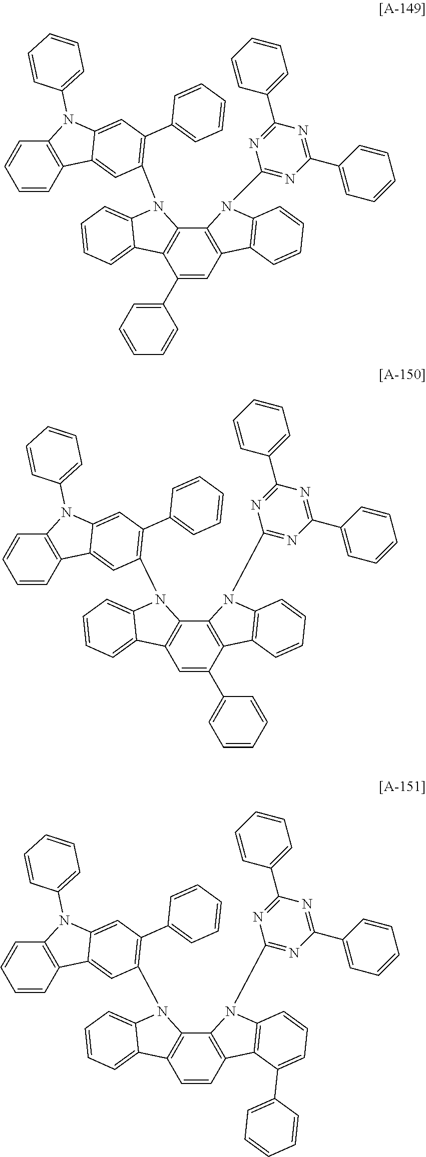 [SMILES: c1ccc(-c2nc(-c3ccccc3)nc(-n3c4ccccc4c4cc(-c5ccccc5)c5c6ccccc6n(-c6cc7c8ccccc8n(-c8ccccc8)c7cc6-c6ccccc6)c5c43)n2)cc1 ; c1ccc(-c2nc(-c3ccccc3)nc(-n3c4ccccc4c4c(-c5ccccc5)cc5c6ccccc6n(-c6cc7c8ccccc8n(-c8ccccc8)c7cc6-c6ccccc6)c5c43)n2)cc1 ; c1ccc(-c2nc(-c3ccccc3)nc(-n3c4cccc(-c5ccccc5)c4c4ccc5c6ccccc6n(-c6cc7c8ccccc8n(-c8ccccc8)c7cc6-c6ccccc6)c5c43)n2)cc1]

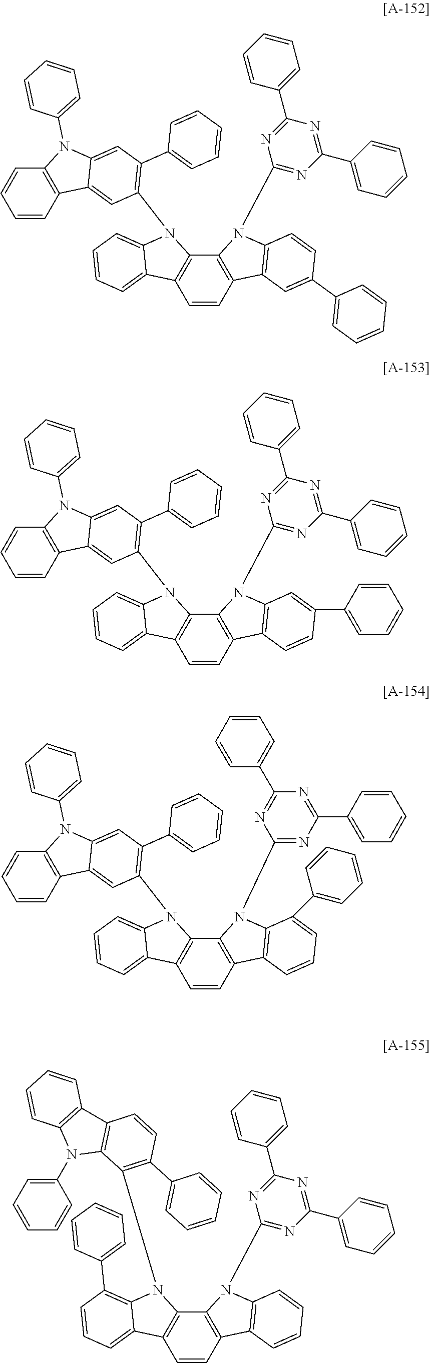 [SMILES: c1ccc(-c2ccc3c(c2)c2ccc4c5ccccc5n(-c5cc6c7ccccc7n(-c7ccccc7)c6cc5-c5ccccc5)c4c2n3-c2nc(-c3ccccc3)nc(-c3ccccc3)n2)cc1 ; c1ccc(-c2ccc3c4ccc5c6ccccc6n(-c6cc7c8ccccc8n(-c8ccccc8)c7cc6-c6ccccc6)c5c4n(-c4nc(-c5ccccc5)nc(-c5ccccc5)n4)c3c2)cc1 ; c1ccc(-c2nc(-c3ccccc3)nc(-n3c4c(-c5ccccc5)cccc4c4ccc5c6ccccc6n(-c6cc7c8ccccc8n(-c8ccccc8)c7cc6-c6ccccc6)c5c43)n2)cc1 ; c1ccc(-c2nc(-c3ccccc3)nc(-n3c4ccccc4c4ccc5c6cccc(-c7ccccc7)c6n(-c6c(-c7ccccc7)ccc7c8ccccc8n(-c8ccccc8)c67)c5c43)n2)cc1]

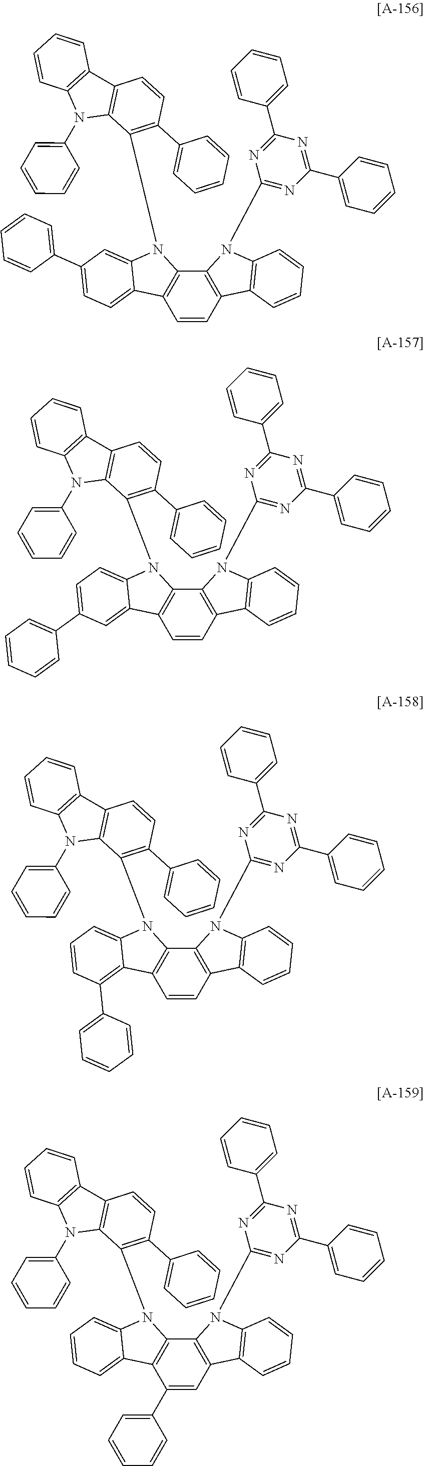 [SMILES: c1ccc(-c2ccc3c4ccc5c6ccccc6n(-c6nc(-c7ccccc7)nc(-c7ccccc7)n6)c5c4n(-c4c(-c5ccccc5)ccc5c6ccccc6n(-c6ccccc6)c45)c3c2)cc1 ; c1ccc(-c2ccc3c(c2)c2ccc4c5ccccc5n(-c5nc(-c6ccccc6)nc(-c6ccccc6)n5)c4c2n3-c2c(-c3ccccc3)ccc3c4ccccc4n(-c4ccccc4)c23)cc1 ; c1ccc(-c2nc(-c3ccccc3)nc(-n3c4ccccc4c4ccc5c6c(-c7ccccc7)cccc6n(-c6c(-c7ccccc7)ccc7c8ccccc8n(-c8ccccc8)c67)c5c43)n2)cc1 ; c1ccc(-c2nc(-c3ccccc3)nc(-n3c4ccccc4c4cc(-c5ccccc5)c5c6ccccc6n(-c6c(-c7ccccc7)ccc7c8ccccc8n(-c8ccccc8)c67)c5c43)n2)cc1]

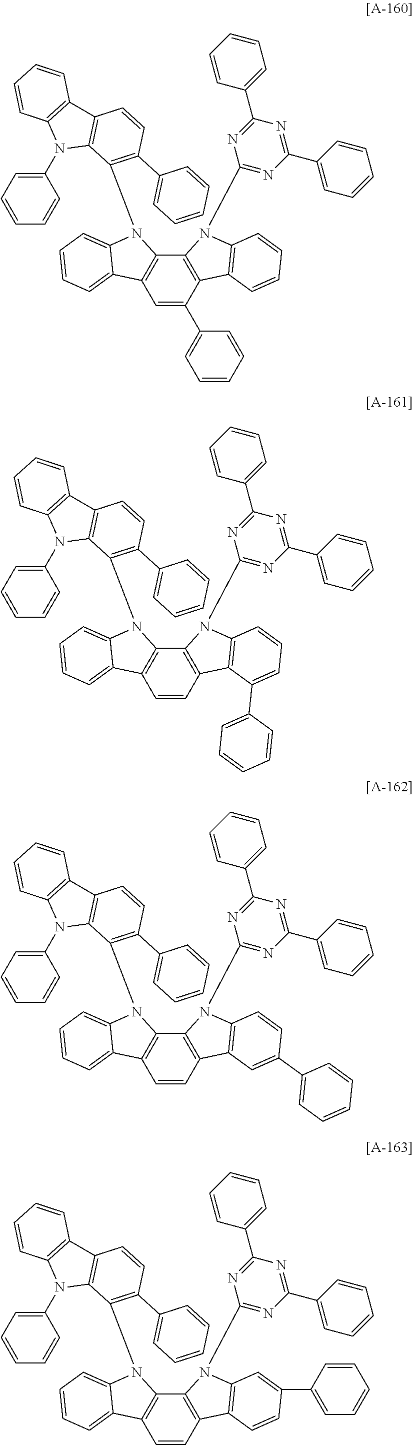 [SMILES: c1ccc(-c2nc(-c3ccccc3)nc(-n3c4ccccc4c4c(-c5ccccc5)cc5c6ccccc6n(-c6c(-c7ccccc7)ccc7c8ccccc8n(-c8ccccc8)c67)c5c43)n2)cc1 ; c1ccc(-c2nc(-c3ccccc3)nc(-n3c4cccc(-c5ccccc5)c4c4ccc5c6ccccc6n(-c6c(-c7ccccc7)ccc7c8ccccc8n(-c8ccccc8)c67)c5c43)n2)cc1 ; c1ccc(-c2ccc3c(c2)c2ccc4c5ccccc5n(-c5c(-c6ccccc6)ccc6c7ccccc7n(-c7ccccc7)c56)c4c2n3-c2nc(-c3ccccc3)nc(-c3ccccc3)n2)cc1 ; c1ccc(-c2ccc3c4ccc5c6ccccc6n(-c6c(-c7ccccc7)ccc7c8ccccc8n(-c8ccccc8)c67)c5c4n(-c4nc(-c5ccccc5)nc(-c5ccccc5)n4)c3c2)cc1]

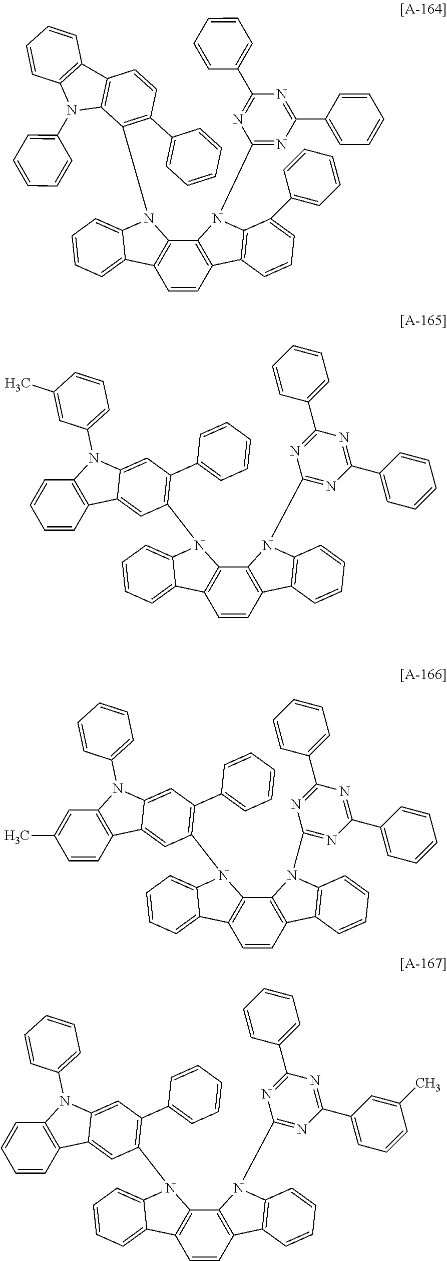 [SMILES: c1ccc(-c2nc(-c3ccccc3)nc(-n3c4c(-c5ccccc5)cccc4c4ccc5c6ccccc6n(-c6c(-c7ccccc7)ccc7c8ccccc8n(-c8ccccc8)c67)c5c43)n2)cc1 ; Cc1cccc(-n2c3ccccc3c3cc(-n4c5ccccc5c5ccc6c7ccccc7n(-c7nc(-c8ccccc8)nc(-c8ccccc8)n7)c6c54)c(-c4ccccc4)cc32)c1 ; Cc1ccc2c3cc(-n4c5ccccc5c5ccc6c7ccccc7n(-c7nc(-c8ccccc8)nc(-c8ccccc8)n7)c6c54)c(-c4ccccc4)cc3n(-c3ccccc3)c2c1 ; Cc1cccc(-c2nc(-c3ccccc3)nc(-n3c4ccccc4c4ccc5c6ccccc6n(-c6cc7c8ccccc8n(-c8ccccc8)c7cc6-c6ccccc6)c5c43)n2)c1]

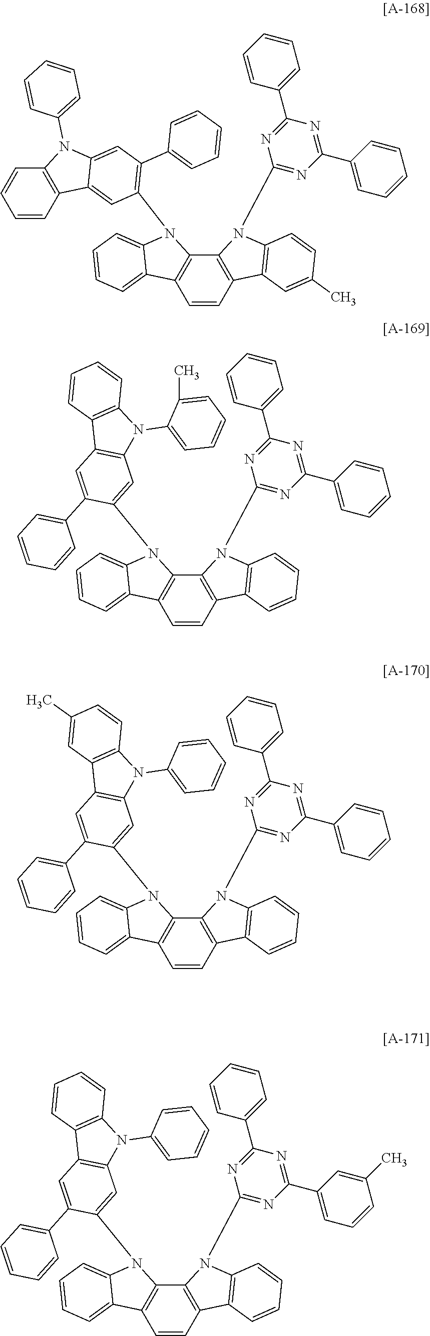 [SMILES: Cc1ccc2c(c1)c1ccc3c4ccccc4n(-c4cc5c6ccccc6n(-c6ccccc6)c5cc4-c4ccccc4)c3c1n2-c1nc(-c2ccccc2)nc(-c2ccccc2)n1 ; Cc1ccccc1-n1c2ccccc2c2cc(-c3ccccc3)c(-n3c4ccccc4c4ccc5c6ccccc6n(-c6nc(-c7ccccc7)nc(-c7ccccc7)n6)c5c43)cc21 ; Cc1ccc2c(c1)c1cc(-c3ccccc3)c(-n3c4ccccc4c4ccc5c6ccccc6n(-c6nc(-c7ccccc7)nc(-c7ccccc7)n6)c5c43)cc1n2-c1ccccc1 ; Cc1cccc(-c2nc(-c3ccccc3)nc(-n3c4ccccc4c4ccc5c6ccccc6n(-c6cc7c(cc6-c6ccccc6)c6ccccc6n7-c6ccccc6)c5c43)n2)c1]

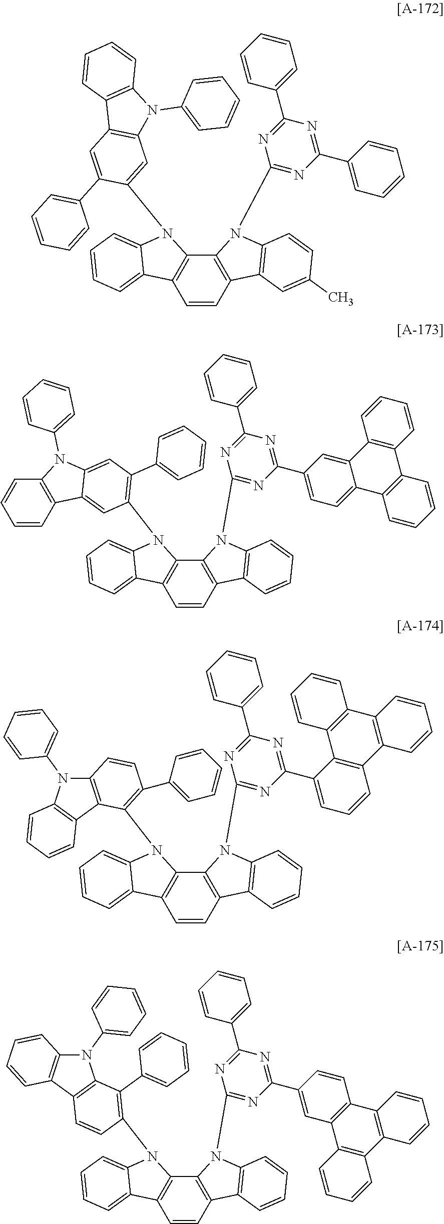 [SMILES: Cc1ccc2c(c1)c1ccc3c4ccccc4n(-c4cc5c(cc4-c4ccccc4)c4ccccc4n5-c4ccccc4)c3c1n2-c1nc(-c2ccccc2)nc(-c2ccccc2)n1 ; c1ccc(-c2nc(-c3ccc4c5ccccc5c5ccccc5c4c3)nc(-n3c4ccccc4c4ccc5c6ccccc6n(-c6cc7c8ccccc8n(-c8ccccc8)c7cc6-c6ccccc6)c5c43)n2)cc1 ; c1ccc(-c2nc(-c3cccc4c5ccccc5c5ccccc5c34)nc(-n3c4ccccc4c4ccc5c6ccccc6n(-c6c(-c7ccccc7)ccc7c6c6ccccc6n7-c6ccccc6)c5c43)n2)cc1 ; c1ccc(-c2nc(-c3ccc4c5ccccc5c5ccccc5c4c3)nc(-n3c4ccccc4c4ccc5c6ccccc6n(-c6ccc7c8ccccc8n(-c8ccccc8)c7c6-c6ccccc6)c5c43)n2)cc1]

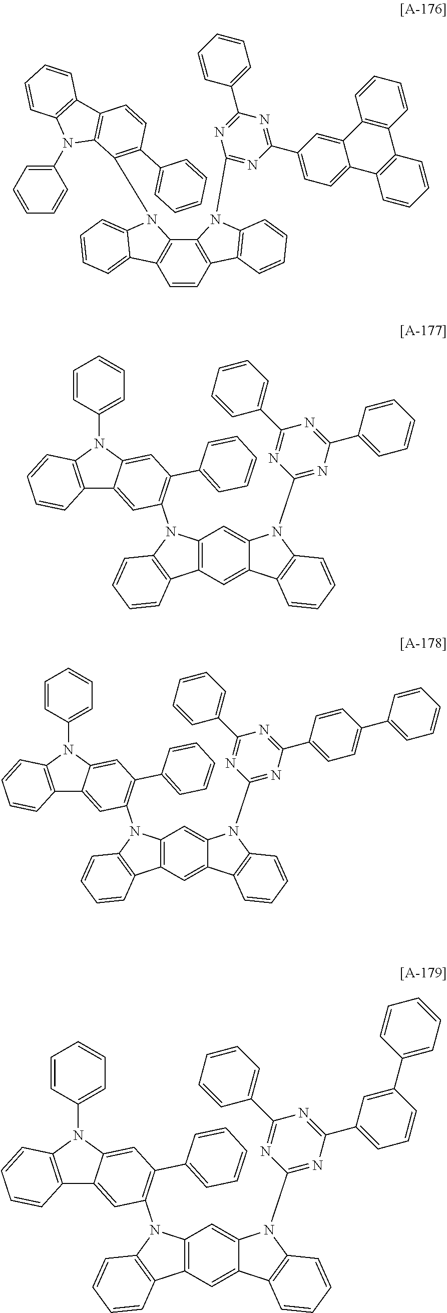 [SMILES: c1ccc(-c2nc(-c3ccc4c5ccccc5c5ccccc5c4c3)nc(-n3c4ccccc4c4ccc5c6ccccc6n(-c6c(-c7ccccc7)ccc7c8ccccc8n(-c8ccccc8)c67)c5c43)n2)cc1 ; c1ccc(-c2nc(-c3ccccc3)nc(-n3c4ccccc4c4cc5c6ccccc6n(-c6cc7c8ccccc8n(-c8ccccc8)c7cc6-c6ccccc6)c5cc43)n2)cc1 ; c1ccc(-c2ccc(-c3nc(-c4ccccc4)nc(-n4c5ccccc5c5cc6c7ccccc7n(-c7cc8c9ccccc9n(-c9ccccc9)c8cc7-c7ccccc7)c6cc54)n3)cc2)cc1 ; c1ccc(-c2cccc(-c3nc(-c4ccccc4)nc(-n4c5ccccc5c5cc6c7ccccc7n(-c7cc8c9ccccc9n(-c9ccccc9)c8cc7-c7ccccc7)c6cc54)n3)c2)cc1]

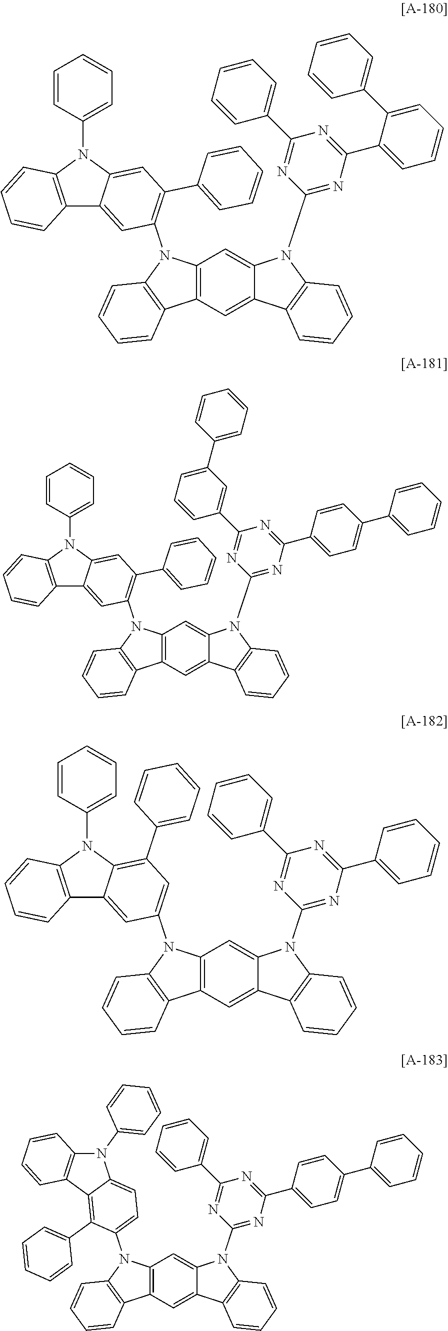 [SMILES: c1ccc(-c2nc(-c3ccccc3-c3ccccc3)nc(-n3c4ccccc4c4cc5c6ccccc6n(-c6cc7c8ccccc8n(-c8ccccc8)c7cc6-c6ccccc6)c5cc43)n2)cc1 ; c1ccc(-c2ccc(-c3nc(-c4cccc(-c5ccccc5)c4)nc(-n4c5ccccc5c5cc6c7ccccc7n(-c7cc8c9ccccc9n(-c9ccccc9)c8cc7-c7ccccc7)c6cc54)n3)cc2)cc1 ; c1ccc(-c2nc(-c3ccccc3)nc(-n3c4ccccc4c4cc5c6ccccc6n(-c6cc(-c7ccccc7)c7c(c6)c6ccccc6n7-c6ccccc6)c5cc43)n2)cc1 ; c1ccc(-c2ccc(-c3nc(-c4ccccc4)nc(-n4c5ccccc5c5cc6c7ccccc7n(-c7ccc8c(c7-c7ccccc7)c7ccccc7n8-c7ccccc7)c6cc54)n3)cc2)cc1]

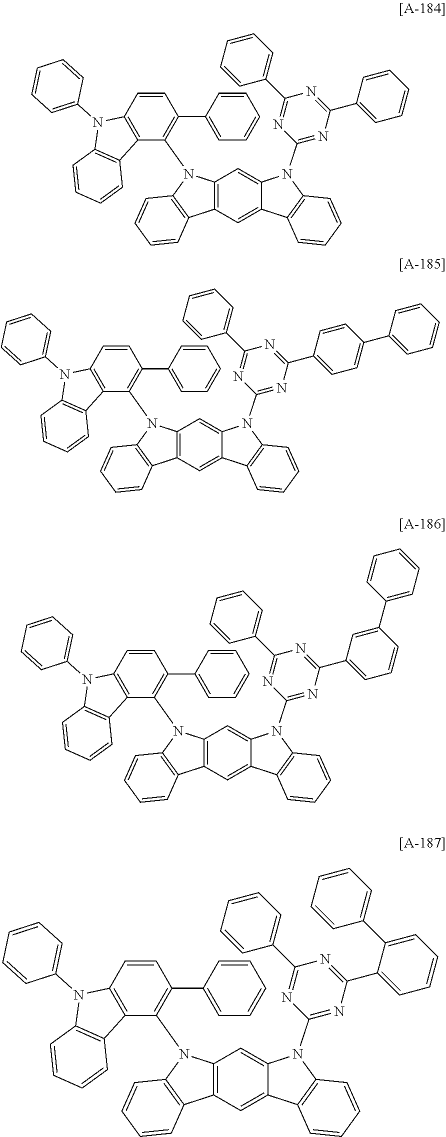 [SMILES: c1ccc(-c2nc(-c3ccccc3)nc(-n3c4ccccc4c4cc5c6ccccc6n(-c6c(-c7ccccc7)ccc7c6c6ccccc6n7-c6ccccc6)c5cc43)n2)cc1 ; c1ccc(-c2ccc(-c3nc(-c4ccccc4)nc(-n4c5ccccc5c5cc6c7ccccc7n(-c7c(-c8ccccc8)ccc8c7c7ccccc7n8-c7ccccc7)c6cc54)n3)cc2)cc1 ; c1ccc(-c2cccc(-c3nc(-c4ccccc4)nc(-n4c5ccccc5c5cc6c7ccccc7n(-c7c(-c8ccccc8)ccc8c7c7ccccc7n8-c7ccccc7)c6cc54)n3)c2)cc1 ; c1ccc(-c2nc(-c3ccccc3-c3ccccc3)nc(-n3c4ccccc4c4cc5c6ccccc6n(-c6c(-c7ccccc7)ccc7c6c6ccccc6n7-c6ccccc6)c5cc43)n2)cc1]

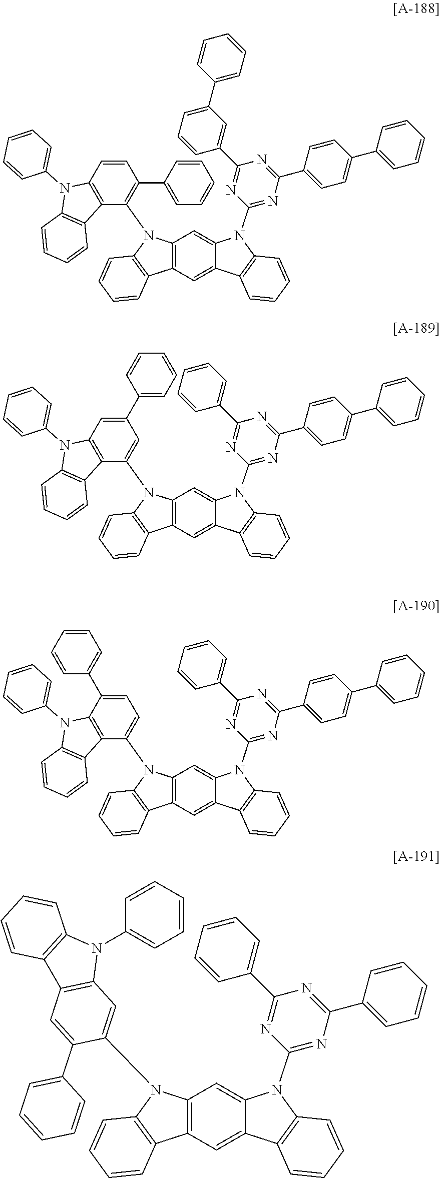 [SMILES: c1ccc(-c2ccc(-c3nc(-c4cccc(-c5ccccc5)c4)nc(-n4c5ccccc5c5cc6c7ccccc7n(-c7c(-c8ccccc8)ccc8c7c7ccccc7n8-c7ccccc7)c6cc54)n3)cc2)cc1 ; c1ccc(-c2ccc(-c3nc(-c4ccccc4)nc(-n4c5ccccc5c5cc6c7ccccc7n(-c7cc(-c8ccccc8)cc8c7c7ccccc7n8-c7ccccc7)c6cc54)n3)cc2)cc1 ; c1ccc(-c2ccc(-c3nc(-c4ccccc4)nc(-n4c5ccccc5c5cc6c7ccccc7n(-c7ccc(-c8ccccc8)c8c7c7ccccc7n8-c7ccccc7)c6cc54)n3)cc2)cc1 ; c1ccc(-c2nc(-c3ccccc3)nc(-n3c4ccccc4c4cc5c6ccccc6n(-c6cc7c(cc6-c6ccccc6)c6ccccc6n7-c6ccccc6)c5cc43)n2)cc1]

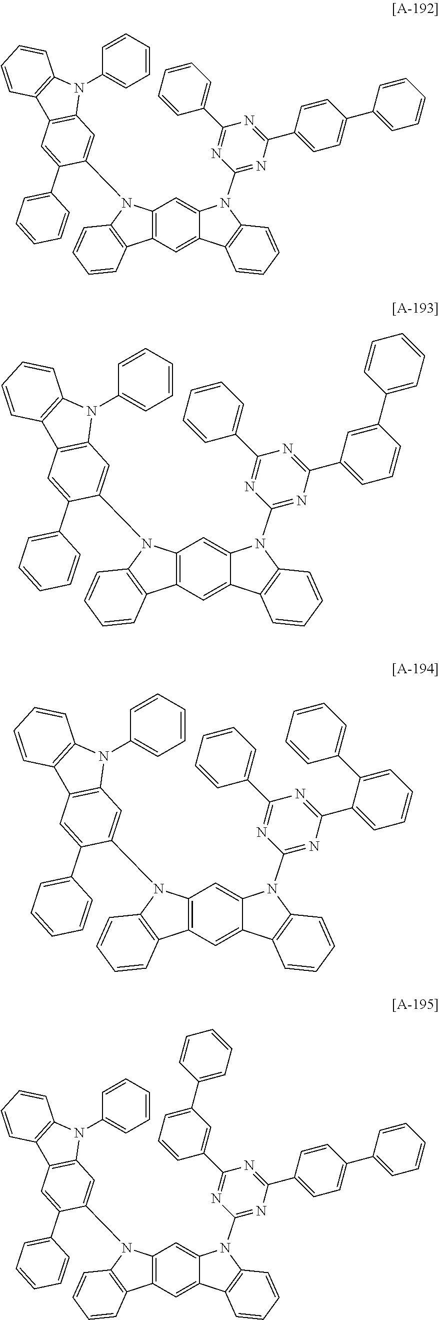 [SMILES: c1ccc(-c2ccc(-c3nc(-c4ccccc4)nc(-n4c5ccccc5c5cc6c7ccccc7n(-c7cc8c(cc7-c7ccccc7)c7ccccc7n8-c7ccccc7)c6cc54)n3)cc2)cc1 ; c1ccc(-c2cccc(-c3nc(-c4ccccc4)nc(-n4c5ccccc5c5cc6c7ccccc7n(-c7cc8c(cc7-c7ccccc7)c7ccccc7n8-c7ccccc7)c6cc54)n3)c2)cc1 ; c1ccc(-c2nc(-c3ccccc3-c3ccccc3)nc(-n3c4ccccc4c4cc5c6ccccc6n(-c6cc7c(cc6-c6ccccc6)c6ccccc6n7-c6ccccc6)c5cc43)n2)cc1 ; c1ccc(-c2ccc(-c3nc(-c4cccc(-c5ccccc5)c4)nc(-n4c5ccccc5c5cc6c7ccccc7n(-c7cc8c(cc7-c7ccccc7)c7ccccc7n8-c7ccccc7)c6cc54)n3)cc2)cc1]

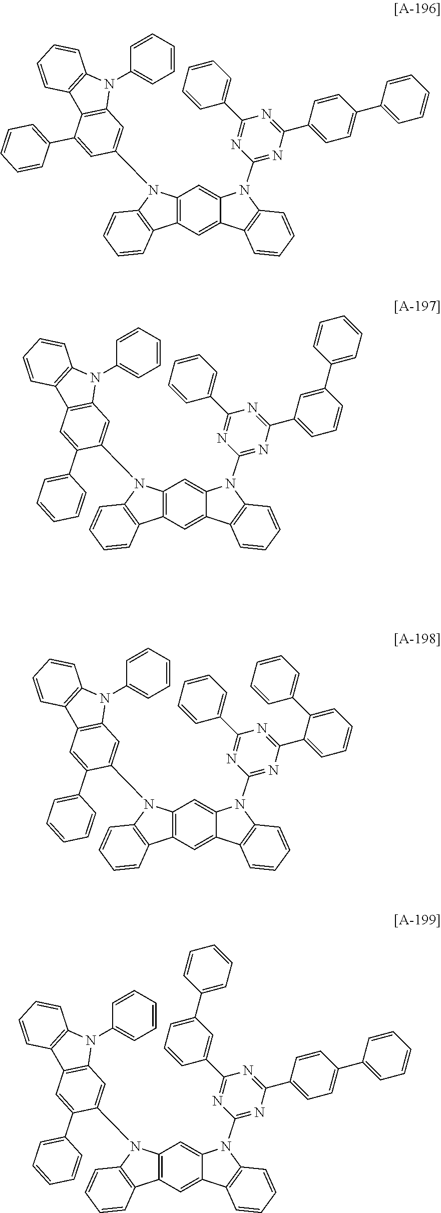 [SMILES: c1ccc(-c2ccc(-c3nc(-c4ccccc4)nc(-n4c5ccccc5c5cc6c7ccccc7n(-c7cc(-c8ccccc8)c8c9ccccc9n(-c9ccccc9)c8c7)c6cc54)n3)cc2)cc1 ; c1ccc(-c2cccc(-c3nc(-c4ccccc4)nc(-n4c5ccccc5c5cc6c7ccccc7n(-c7cc8c(cc7-c7ccccc7)c7ccccc7n8-c7ccccc7)c6cc54)n3)c2)cc1 ; c1ccc(-c2nc(-c3ccccc3-c3ccccc3)nc(-n3c4ccccc4c4cc5c6ccccc6n(-c6cc7c(cc6-c6ccccc6)c6ccccc6n7-c6ccccc6)c5cc43)n2)cc1 ; c1ccc(-c2ccc(-c3nc(-c4cccc(-c5ccccc5)c4)nc(-n4c5ccccc5c5cc6c7ccccc7n(-c7cc8c(cc7-c7ccccc7)c7ccccc7n8-c7ccccc7)c6cc54)n3)cc2)cc1]

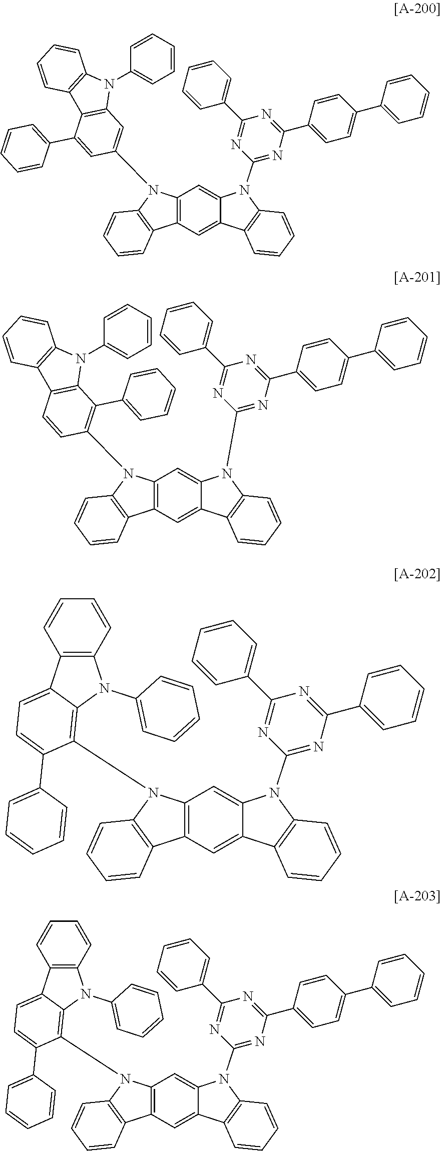 [SMILES: c1ccc(-c2ccc(-c3nc(-c4ccccc4)nc(-n4c5ccccc5c5cc6c7ccccc7n(-c7cc(-c8ccccc8)c8c9ccccc9n(-c9ccccc9)c8c7)c6cc54)n3)cc2)cc1 ; c1ccc(-c2ccc(-c3nc(-c4ccccc4)nc(-n4c5ccccc5c5cc6c7ccccc7n(-c7ccc8c9ccccc9n(-c9ccccc9)c8c7-c7ccccc7)c6cc54)n3)cc2)cc1 ; c1ccc(-c2nc(-c3ccccc3)nc(-n3c4ccccc4c4cc5c6ccccc6n(-c6c(-c7ccccc7)ccc7c8ccccc8n(-c8ccccc8)c67)c5cc43)n2)cc1 ; c1ccc(-c2ccc(-c3nc(-c4ccccc4)nc(-n4c5ccccc5c5cc6c7ccccc7n(-c7c(-c8ccccc8)ccc8c9ccccc9n(-c9ccccc9)c78)c6cc54)n3)cc2)cc1]

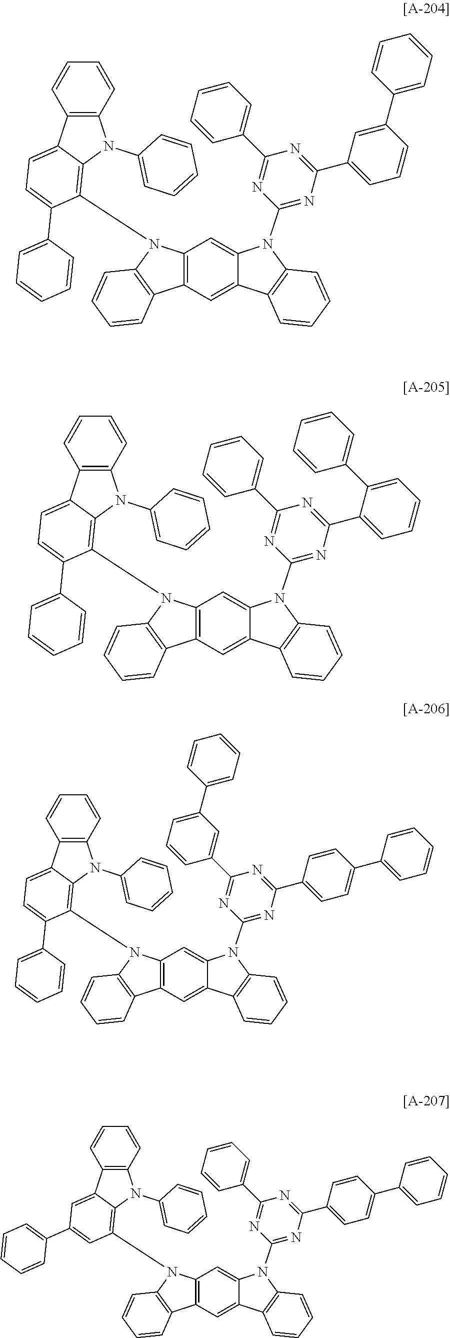 [SMILES: c1ccc(-c2cccc(-c3nc(-c4ccccc4)nc(-n4c5ccccc5c5cc6c7ccccc7n(-c7c(-c8ccccc8)ccc8c9ccccc9n(-c9ccccc9)c78)c6cc54)n3)c2)cc1 ; c1ccc(-c2nc(-c3ccccc3-c3ccccc3)nc(-n3c4ccccc4c4cc5c6ccccc6n(-c6c(-c7ccccc7)ccc7c8ccccc8n(-c8ccccc8)c67)c5cc43)n2)cc1 ; c1ccc(-c2ccc(-c3nc(-c4cccc(-c5ccccc5)c4)nc(-n4c5ccccc5c5cc6c7ccccc7n(-c7c(-c8ccccc8)ccc8c9ccccc9n(-c9ccccc9)c78)c6cc54)n3)cc2)cc1 ; c1ccc(-c2ccc(-c3nc(-c4ccccc4)nc(-n4c5ccccc5c5cc6c7ccccc7n(-c7cc(-c8ccccc8)cc8c9ccccc9n(-c9ccccc9)c78)c6cc54)n3)cc2)cc1]

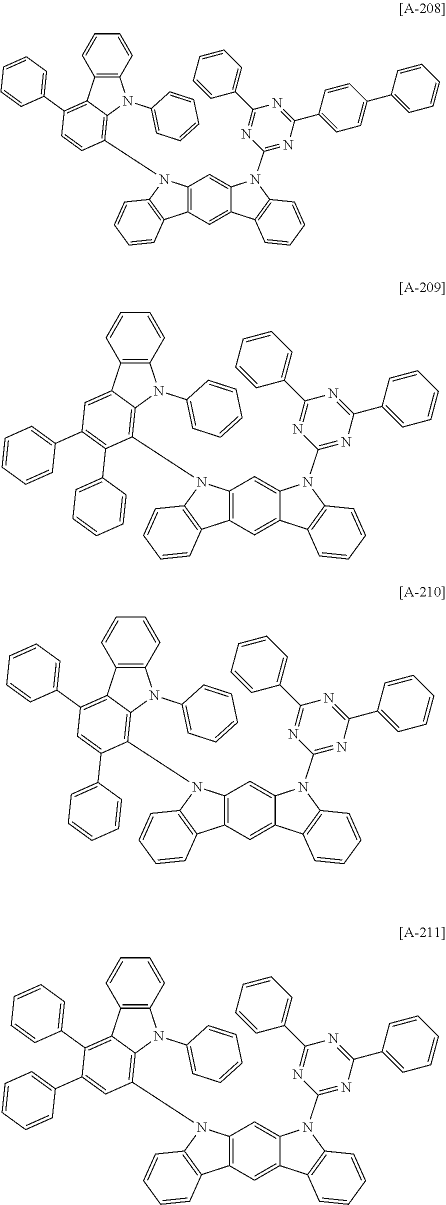 [SMILES: c1ccc(-c2ccc(-c3nc(-c4ccccc4)nc(-n4c5ccccc5c5cc6c7ccccc7n(-c7ccc(-c8ccccc8)c8c9ccccc9n(-c9ccccc9)c78)c6cc54)n3)cc2)cc1 ; c1ccc(-c2nc(-c3ccccc3)nc(-n3c4ccccc4c4cc5c6ccccc6n(-c6c(-c7ccccc7)c(-c7ccccc7)cc7c8ccccc8n(-c8ccccc8)c67)c5cc43)n2)cc1 ; c1ccc(-c2nc(-c3ccccc3)nc(-n3c4ccccc4c4cc5c6ccccc6n(-c6c(-c7ccccc7)cc(-c7ccccc7)c7c8ccccc8n(-c8ccccc8)c67)c5cc43)n2)cc1 ; c1ccc(-c2nc(-c3ccccc3)nc(-n3c4ccccc4c4cc5c6ccccc6n(-c6cc(-c7ccccc7)c(-c7ccccc7)c7c8ccccc8n(-c8ccccc8)c67)c5cc43)n2)cc1]

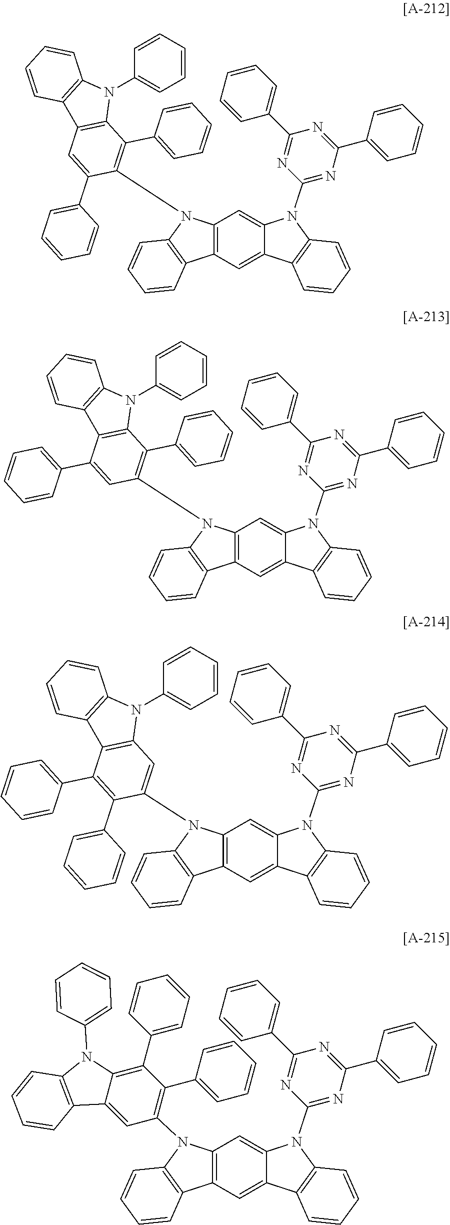 [SMILES: c1ccc(-c2nc(-c3ccccc3)nc(-n3c4ccccc4c4cc5c6ccccc6n(-c6c(-c7ccccc7)cc7c8ccccc8n(-c8ccccc8)c7c6-c6ccccc6)c5cc43)n2)cc1 ; c1ccc(-c2nc(-c3ccccc3)nc(-n3c4ccccc4c4cc5c6ccccc6n(-c6cc(-c7ccccc7)c7c8ccccc8n(-c8ccccc8)c7c6-c6ccccc6)c5cc43)n2)cc1 ; c1ccc(-c2nc(-c3ccccc3)nc(-n3c4ccccc4c4cc5c6ccccc6n(-c6cc7c(c(-c8ccccc8)c6-c6ccccc6)c6ccccc6n7-c6ccccc6)c5cc43)n2)cc1 ; c1ccc(-c2nc(-c3ccccc3)nc(-n3c4ccccc4c4cc5c6ccccc6n(-c6cc7c8ccccc8n(-c8ccccc8)c7c(-c7ccccc7)c6-c6ccccc6)c5cc43)n2)cc1]

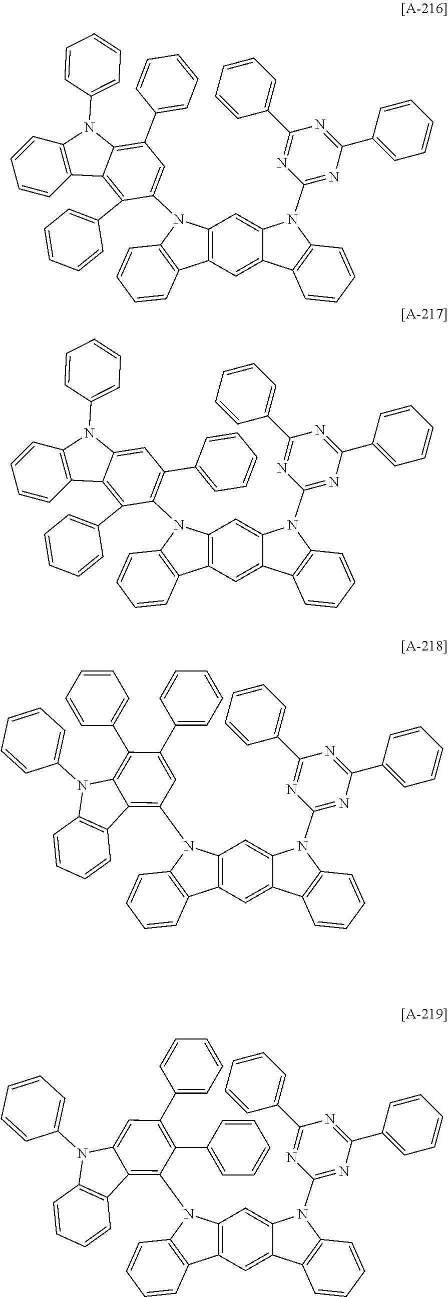 [SMILES: c1ccc(-c2nc(-c3ccccc3)nc(-n3c4ccccc4c4cc5c6ccccc6n(-c6cc(-c7ccccc7)c7c(c6-c6ccccc6)c6ccccc6n7-c6ccccc6)c5cc43)n2)cc1 ; c1ccc(-c2nc(-c3ccccc3)nc(-n3c4ccccc4c4cc5c6ccccc6n(-c6c(-c7ccccc7)cc7c(c6-c6ccccc6)c6ccccc6n7-c6ccccc6)c5cc43)n2)cc1 ; c1ccc(-c2nc(-c3ccccc3)nc(-n3c4ccccc4c4cc5c6ccccc6n(-c6cc(-c7ccccc7)c(-c7ccccc7)c7c6c6ccccc6n7-c6ccccc6)c5cc43)n2)cc1 ; c1ccc(-c2nc(-c3ccccc3)nc(-n3c4ccccc4c4cc5c6ccccc6n(-c6c(-c7ccccc7)c(-c7ccccc7)cc7c6c6ccccc6n7-c6ccccc6)c5cc43)n2)cc1]

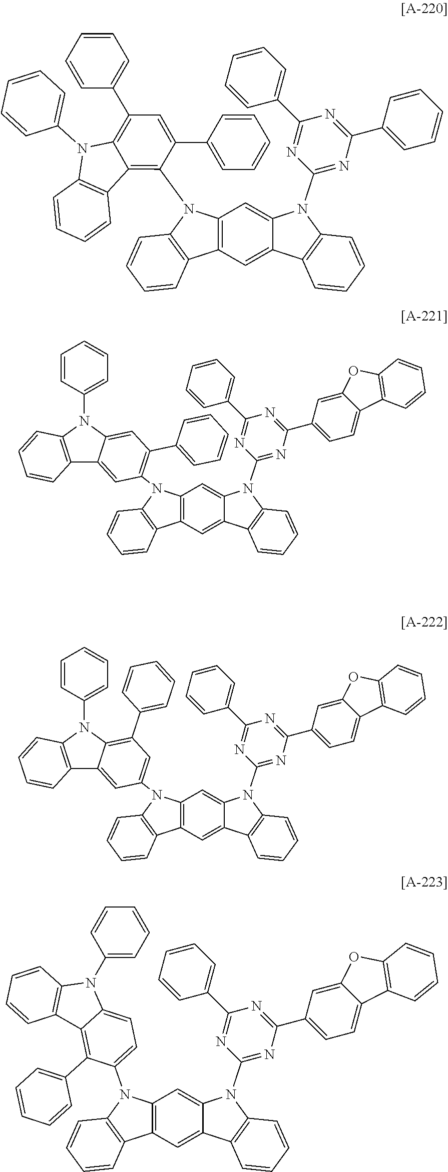 [SMILES: c1ccc(-c2nc(-c3ccccc3)nc(-n3c4ccccc4c4cc5c6ccccc6n(-c6c(-c7ccccc7)cc(-c7ccccc7)c7c6c6ccccc6n7-c6ccccc6)c5cc43)n2)cc1 ; c1ccc(-c2nc(-c3ccc4c(c3)oc3ccccc34)nc(-n3c4ccccc4c4cc5c6ccccc6n(-c6cc7c8ccccc8n(-c8ccccc8)c7cc6-c6ccccc6)c5cc43)n2)cc1 ; c1ccc(-c2nc(-c3ccc4c(c3)oc3ccccc34)nc(-n3c4ccccc4c4cc5c6ccccc6n(-c6cc(-c7ccccc7)c7c(c6)c6ccccc6n7-c6ccccc6)c5cc43)n2)cc1 ; c1ccc(-c2nc(-c3ccc4c(c3)oc3ccccc34)nc(-n3c4ccccc4c4cc5c6ccccc6n(-c6ccc7c(c6-c6ccccc6)c6ccccc6n7-c6ccccc6)c5cc43)n2)cc1]

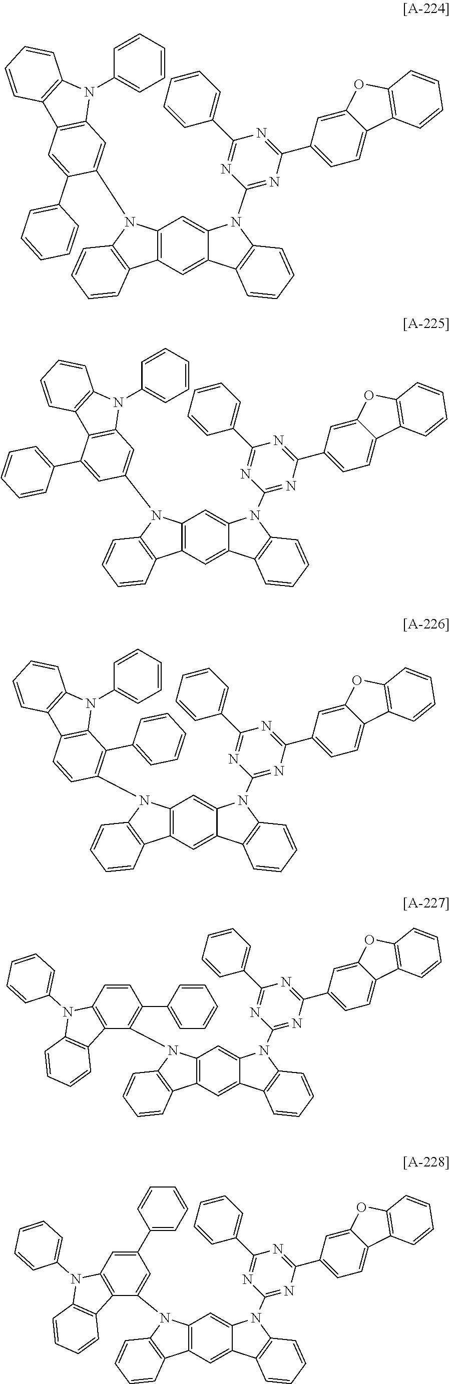

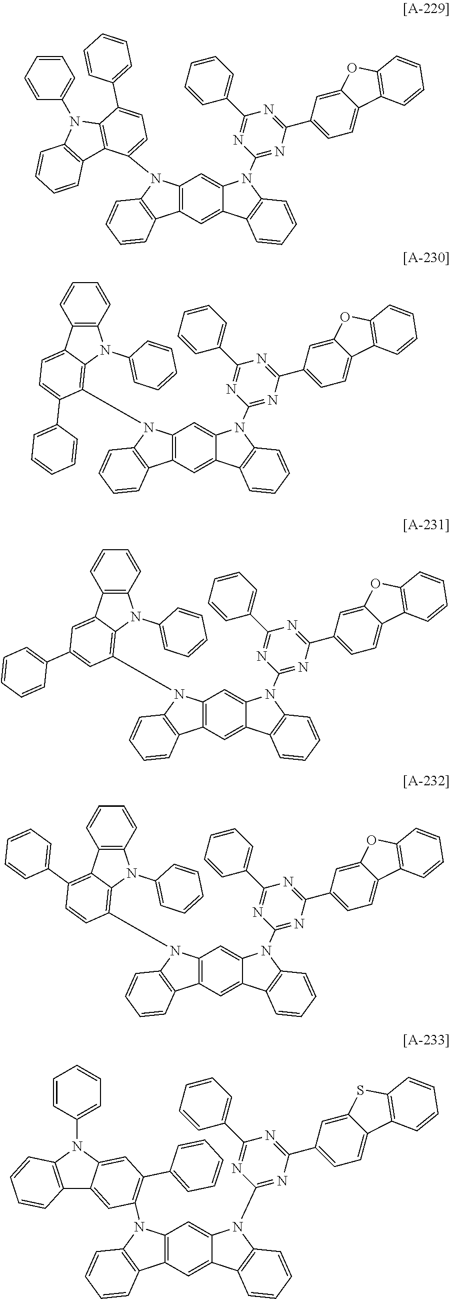

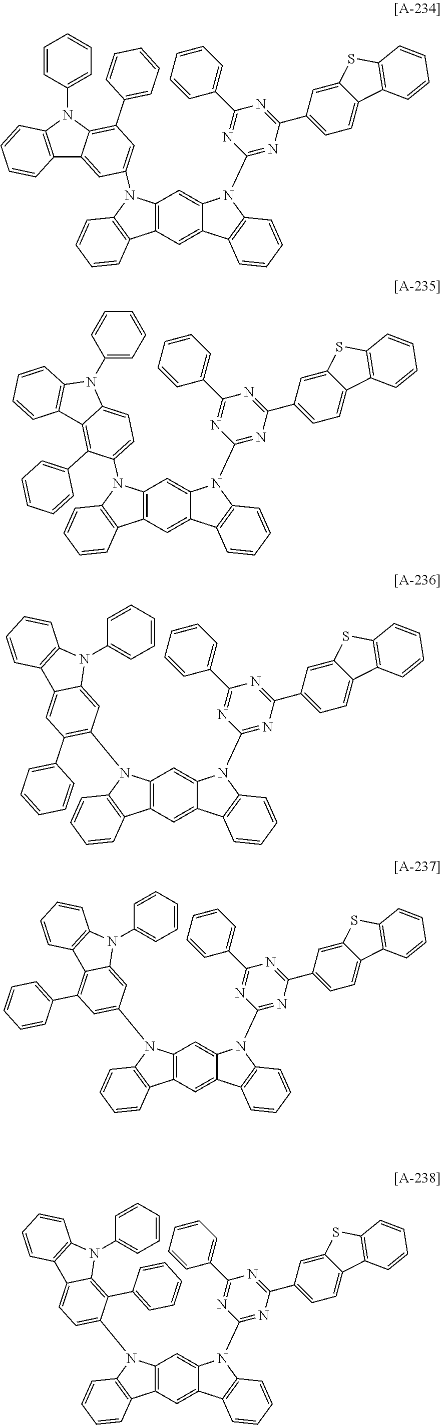

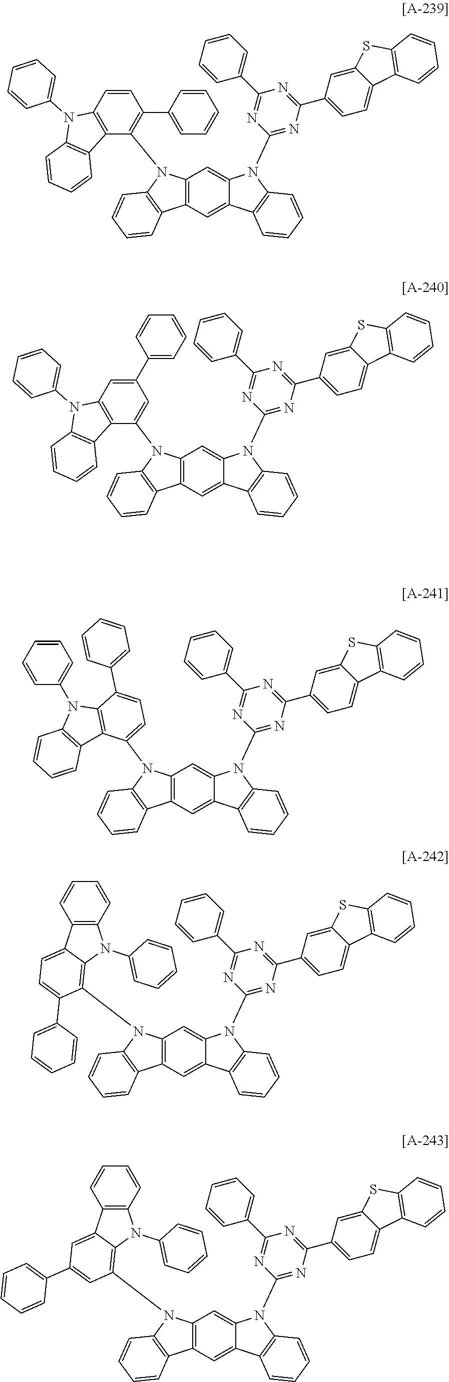

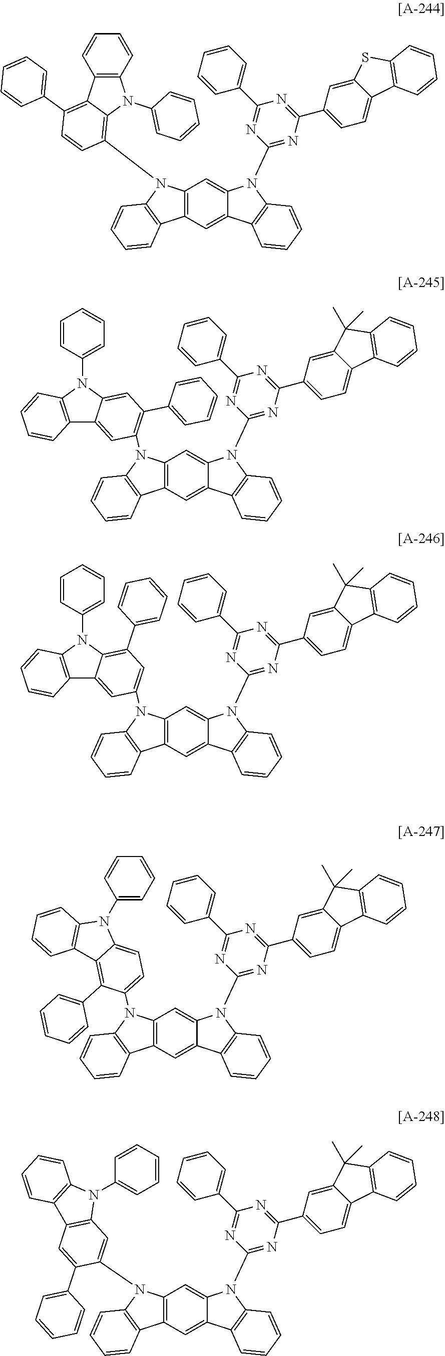

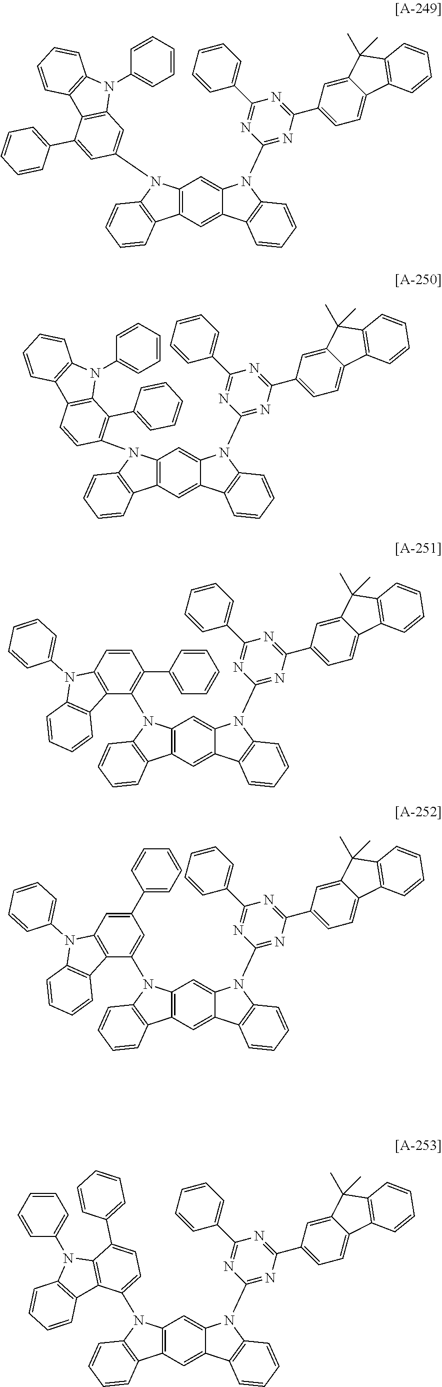

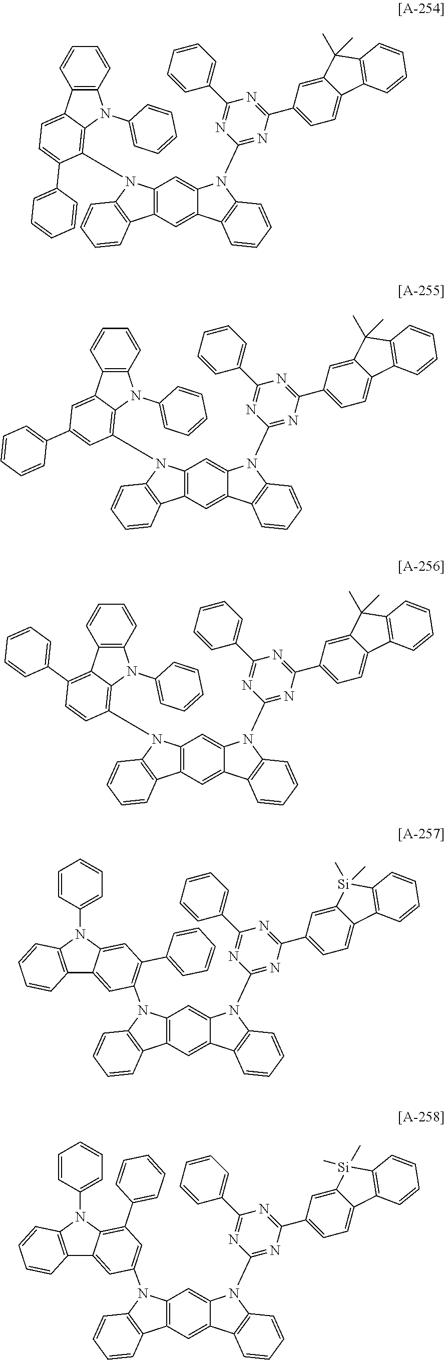

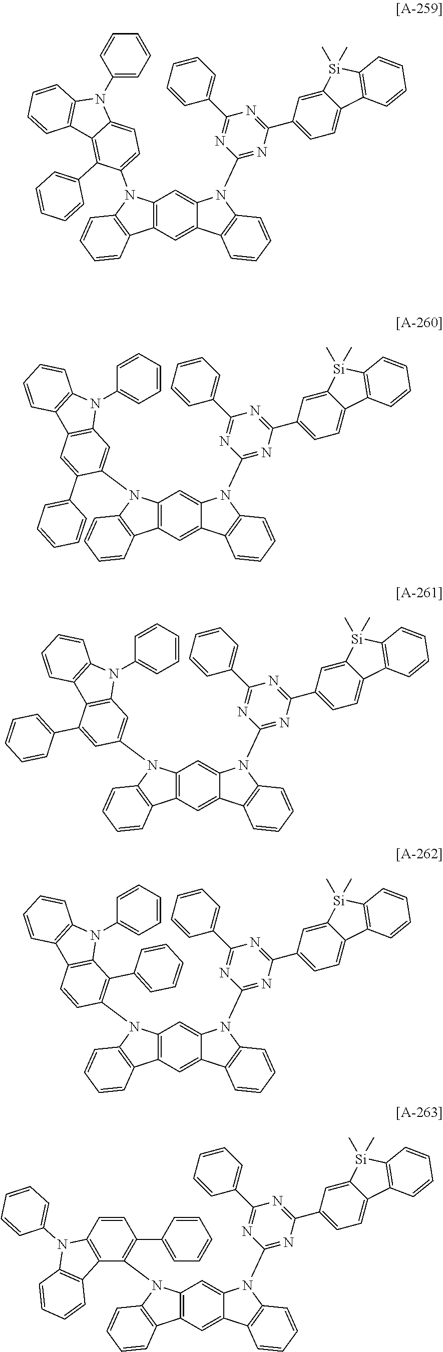

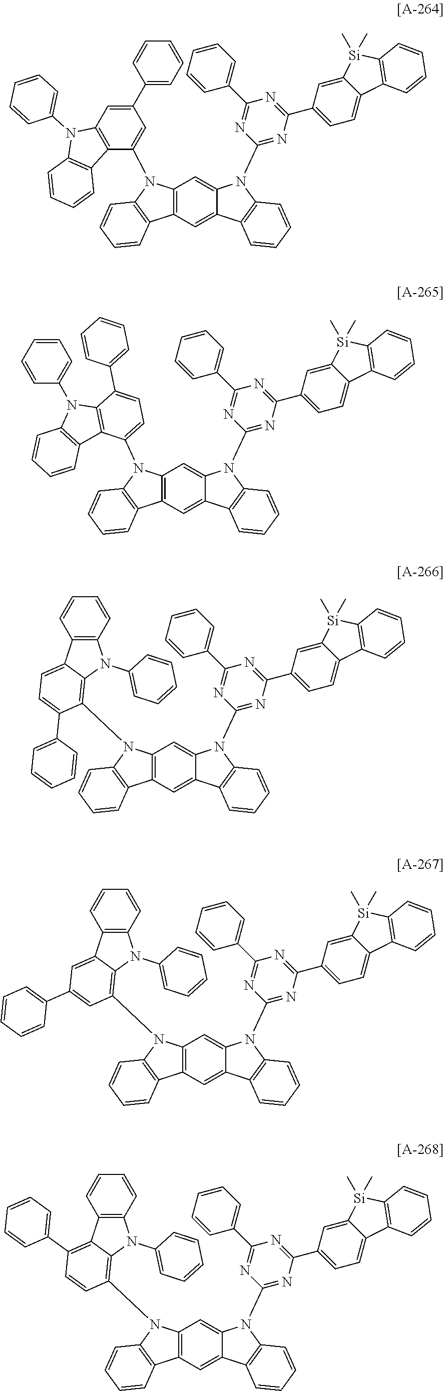

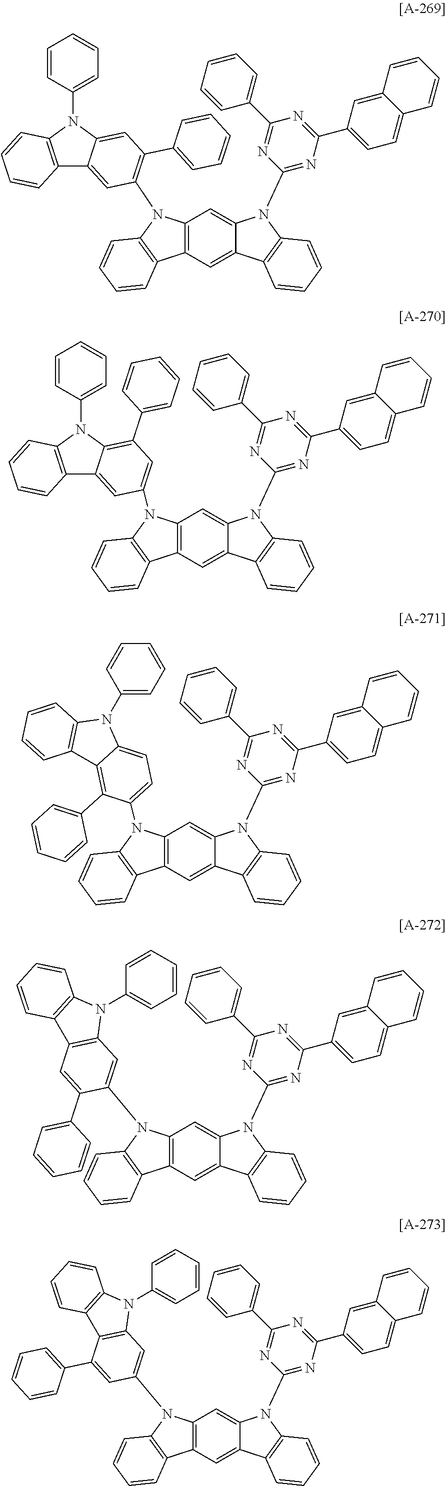

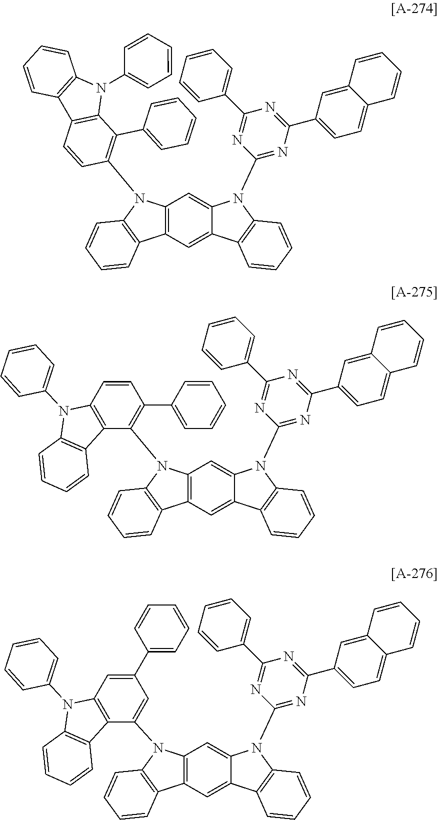 [SMILES: c1ccc(-c2nc(-c3ccc4ccccc4c3)nc(-n3c4ccccc4c4cc5c6ccccc6n(-c6ccc7c8ccccc8n(-c8ccccc8)c7c6-c6ccccc6)c5cc43)n2)cc1 ; c1ccc(-c2nc(-c3ccc4ccccc4c3)nc(-n3c4ccccc4c4cc5c6ccccc6n(-c6c(-c7ccccc7)ccc7c6c6ccccc6n7-c6ccccc6)c5cc43)n2)cc1 ; c1ccc(-c2cc(-n3c4ccccc4c4cc5c6ccccc6n(-c6nc(-c7ccccc7)nc(-c7ccc8ccccc8c7)n6)c5cc43)c3c4ccccc4n(-c4ccccc4)c3c2)cc1]

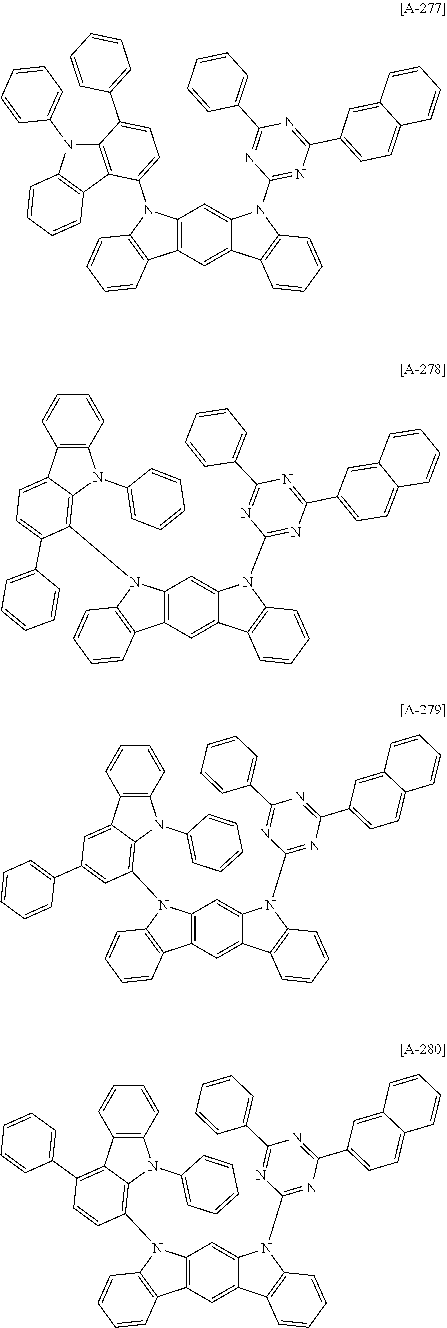 [SMILES: c1ccc(-c2nc(-c3ccc4ccccc4c3)nc(-n3c4ccccc4c4cc5c6ccccc6n(-c6ccc(-c7ccccc7)c7c6c6ccccc6n7-c6ccccc6)c5cc43)n2)cc1 ; c1ccc(-c2nc(-c3ccc4ccccc4c3)nc(-n3c4ccccc4c4cc5c6ccccc6n(-c6c(-c7ccccc7)ccc7c8ccccc8n(-c8ccccc8)c67)c5cc43)n2)cc1 ; c1ccc(-c2cc(-n3c4ccccc4c4cc5c6ccccc6n(-c6nc(-c7ccccc7)nc(-c7ccc8ccccc8c7)n6)c5cc43)c3c(c2)c2ccccc2n3-c2ccccc2)cc1 ; c1ccc(-c2nc(-c3ccc4ccccc4c3)nc(-n3c4ccccc4c4cc5c6ccccc6n(-c6ccc(-c7ccccc7)c7c8ccccc8n(-c8ccccc8)c67)c5cc43)n2)cc1]

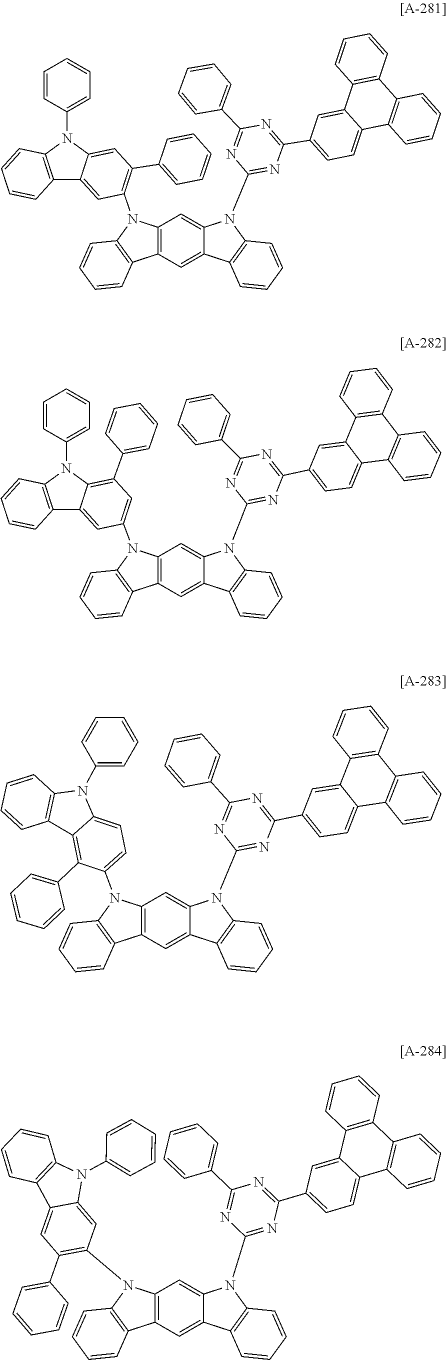

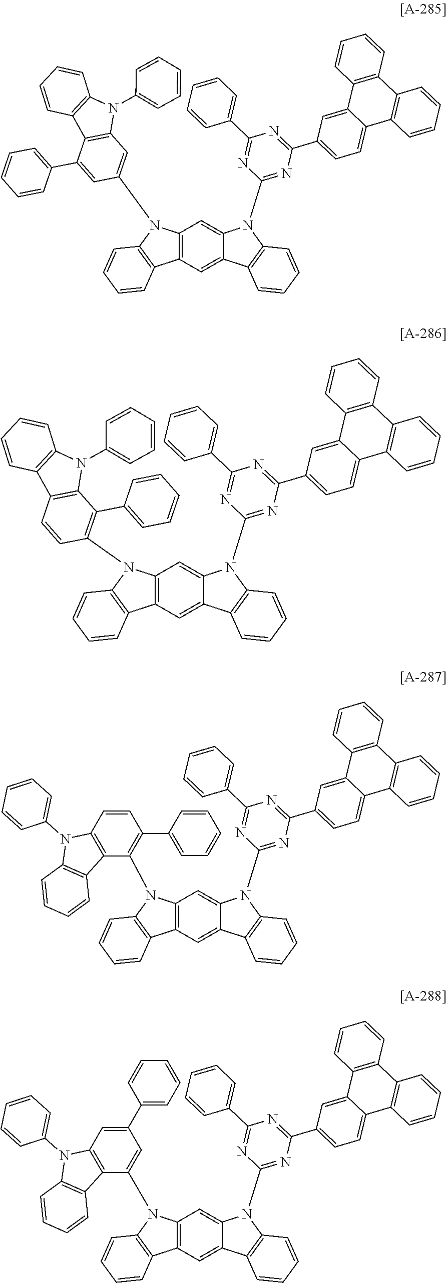

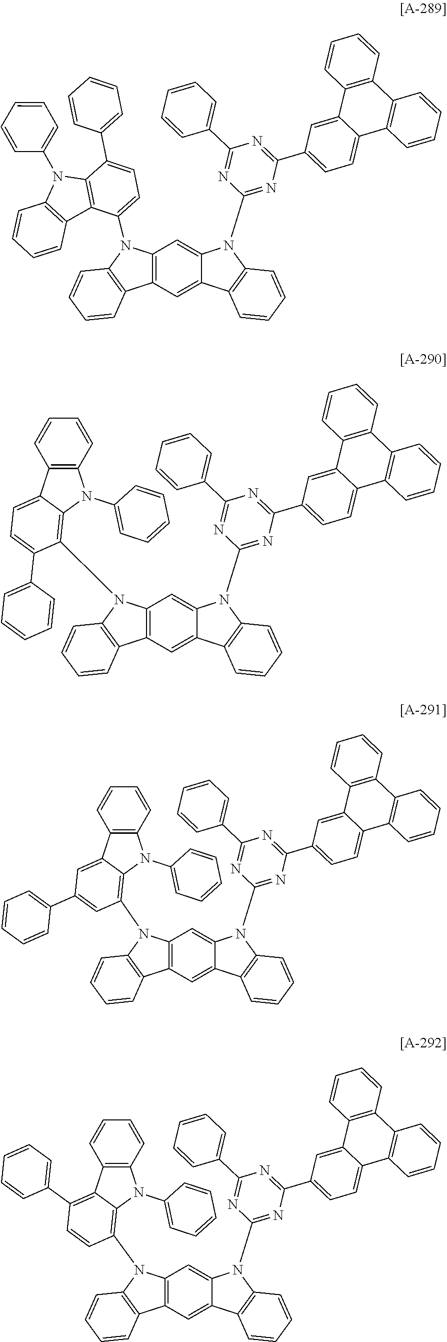

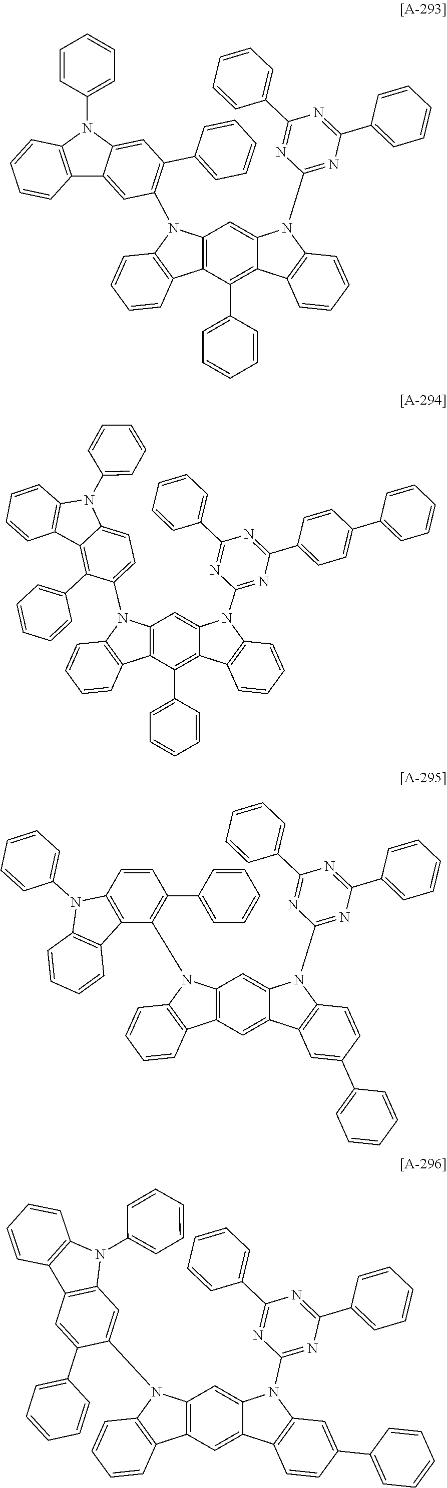 [SMILES: c1ccc(-c2nc(-c3ccccc3)nc(-n3c4ccccc4c4c(-c5ccccc5)c5c6ccccc6n(-c6cc7c8ccccc8n(-c8ccccc8)c7cc6-c6ccccc6)c5cc43)n2)cc1 ; c1ccc(-c2ccc(-c3nc(-c4ccccc4)nc(-n4c5ccccc5c5c(-c6ccccc6)c6c7ccccc7n(-c7ccc8c(c7-c7ccccc7)c7ccccc7n8-c7ccccc7)c6cc54)n3)cc2)cc1 ; c1ccc(-c2ccc3c(c2)c2cc4c5ccccc5n(-c5c(-c6ccccc6)ccc6c5c5ccccc5n6-c5ccccc5)c4cc2n3-c2nc(-c3ccccc3)nc(-c3ccccc3)n2)cc1 ; c1ccc(-c2ccc3c4cc5c6ccccc6n(-c6cc7c(cc6-c6ccccc6)c6ccccc6n7-c6ccccc6)c5cc4n(-c4nc(-c5ccccc5)nc(-c5ccccc5)n4)c3c2)cc1]

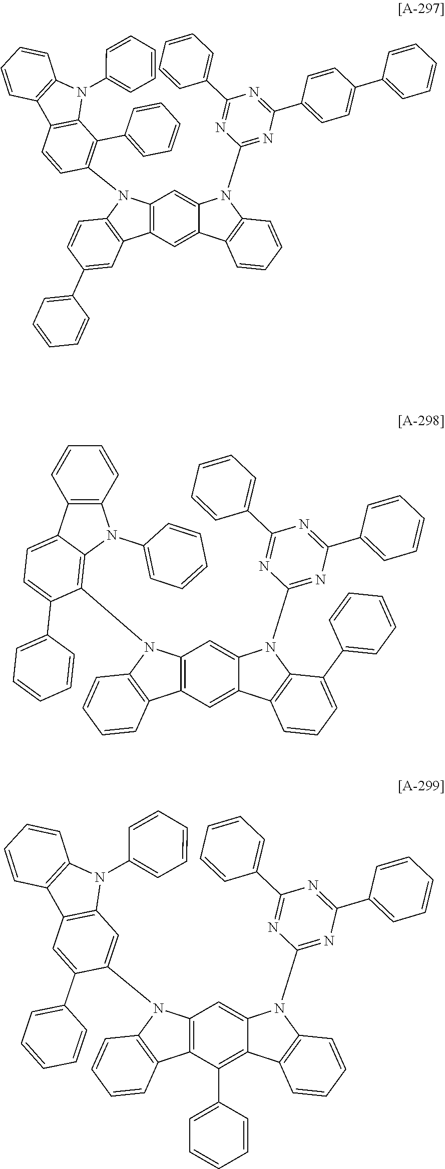 [SMILES: c1ccc(-c2ccc(-c3nc(-c4ccccc4)nc(-n4c5ccccc5c5cc6c7cc(-c8ccccc8)ccc7n(-c7ccc8c9ccccc9n(-c9ccccc9)c8c7-c7ccccc7)c6cc54)n3)cc2)cc1 ; c1ccc(-c2nc(-c3ccccc3)nc(-n3c4cc5c(cc4c4cccc(-c6ccccc6)c43)c3ccccc3n5-c3c(-c4ccccc4)ccc4c5ccccc5n(-c5ccccc5)c34)n2)cc1 ; c1ccc(-c2nc(-c3ccccc3)nc(-n3c4ccccc4c4c(-c5ccccc5)c5c6ccccc6n(-c6cc7c(cc6-c6ccccc6)c6ccccc6n7-c6ccccc6)c5cc43)n2)cc1]

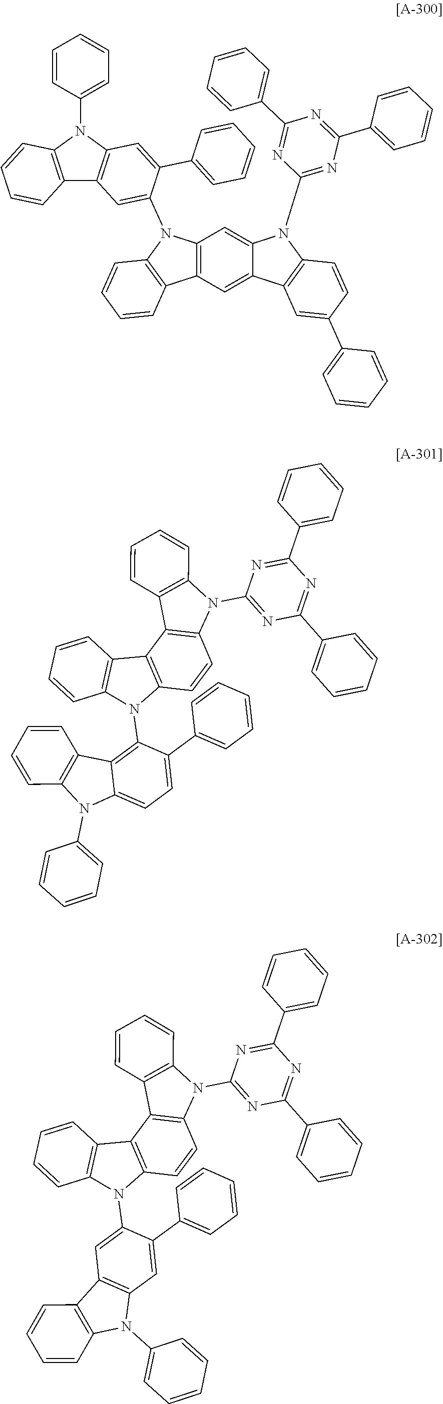 [SMILES: c1ccc(-c2ccc3c(c2)c2cc4c5ccccc5n(-c5cc6c7ccccc7n(-c7ccccc7)c6cc5-c5ccccc5)c4cc2n3-c2nc(-c3ccccc3)nc(-c3ccccc3)n2)cc1 ; c1ccc(-c2nc(-c3ccccc3)nc(-n3c4ccccc4c4c5c6ccccc6n(-c6c(-c7ccccc7)ccc7c6c6ccccc6n7-c6ccccc6)c5ccc43)n2)cc1 ; c1ccc(-c2nc(-c3ccccc3)nc(-n3c4ccccc4c4c5c6ccccc6n(-c6cc7c8ccccc8n(-c8ccccc8)c7cc6-c6ccccc6)c5ccc43)n2)cc1]

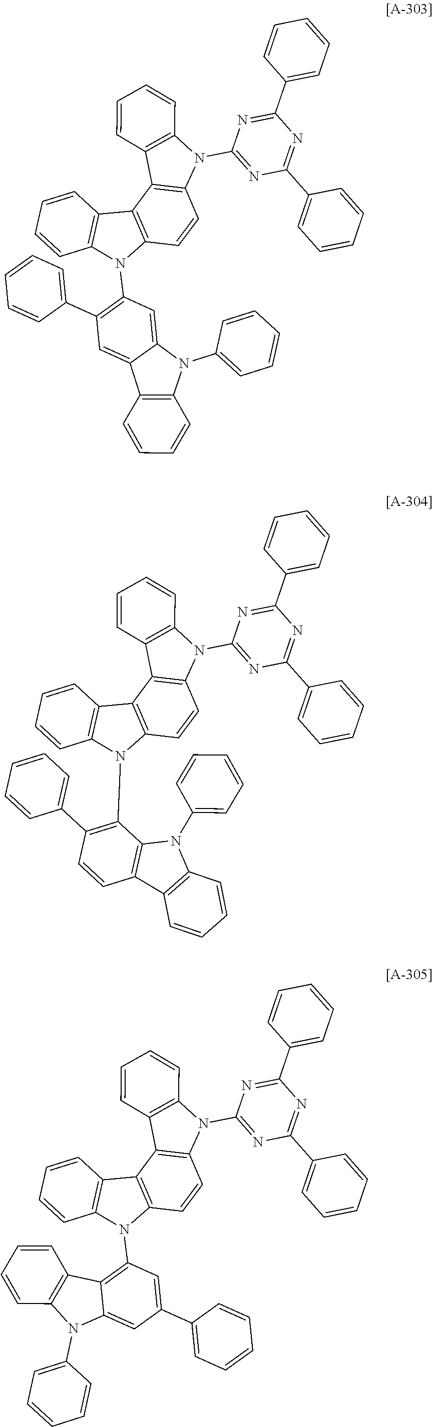 [SMILES: c1ccc(-c2nc(-c3ccccc3)nc(-n3c4ccccc4c4c5c6ccccc6n(-c6cc7c(cc6-c6ccccc6)c6ccccc6n7-c6ccccc6)c5ccc43)n2)cc1 ; c1ccc(-c2nc(-c3ccccc3)nc(-n3c4ccccc4c4c5c6ccccc6n(-c6c(-c7ccccc7)ccc7c8ccccc8n(-c8ccccc8)c67)c5ccc43)n2)cc1 ; c1ccc(-c2cc(-n3c4ccccc4c4c5c6ccccc6n(-c6nc(-c7ccccc7)nc(-c7ccccc7)n6)c5ccc43)c3c4ccccc4n(-c4ccccc4)c3c2)cc1]

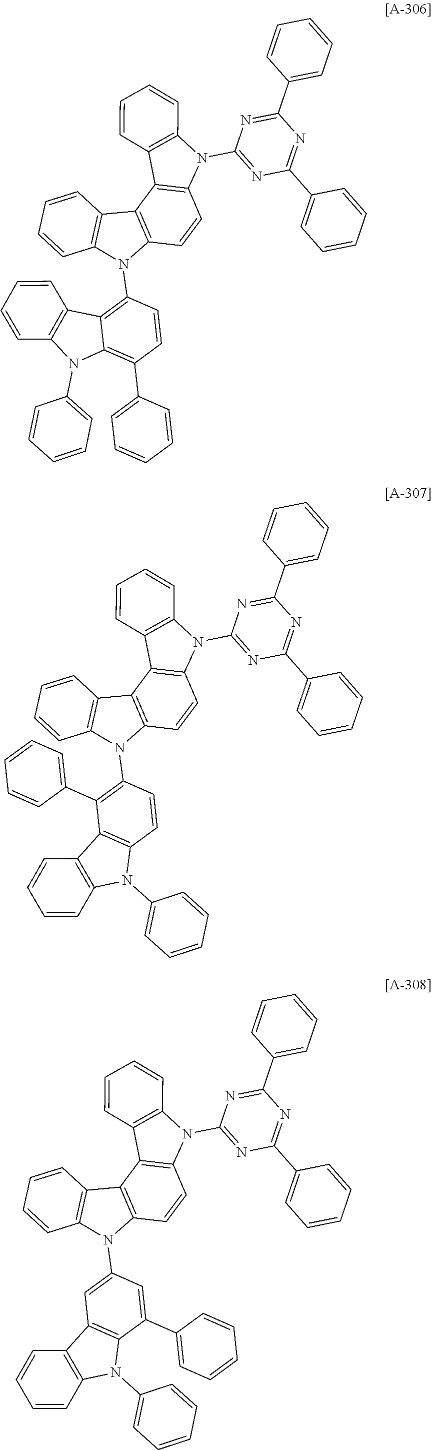 [SMILES: c1ccc(-c2nc(-c3ccccc3)nc(-n3c4ccccc4c4c5c6ccccc6n(-c6ccc(-c7ccccc7)c7c6c6ccccc6n7-c6ccccc6)c5ccc43)n2)cc1 ; c1ccc(-c2nc(-c3ccccc3)nc(-n3c4ccccc4c4c5c6ccccc6n(-c6ccc7c(c6-c6ccccc6)c6ccccc6n7-c6ccccc6)c5ccc43)n2)cc1 ; c1ccc(-c2nc(-c3ccccc3)nc(-n3c4ccccc4c4c5c6ccccc6n(-c6cc(-c7ccccc7)c7c(c6)c6ccccc6n7-c6ccccc6)c5ccc43)n2)cc1]

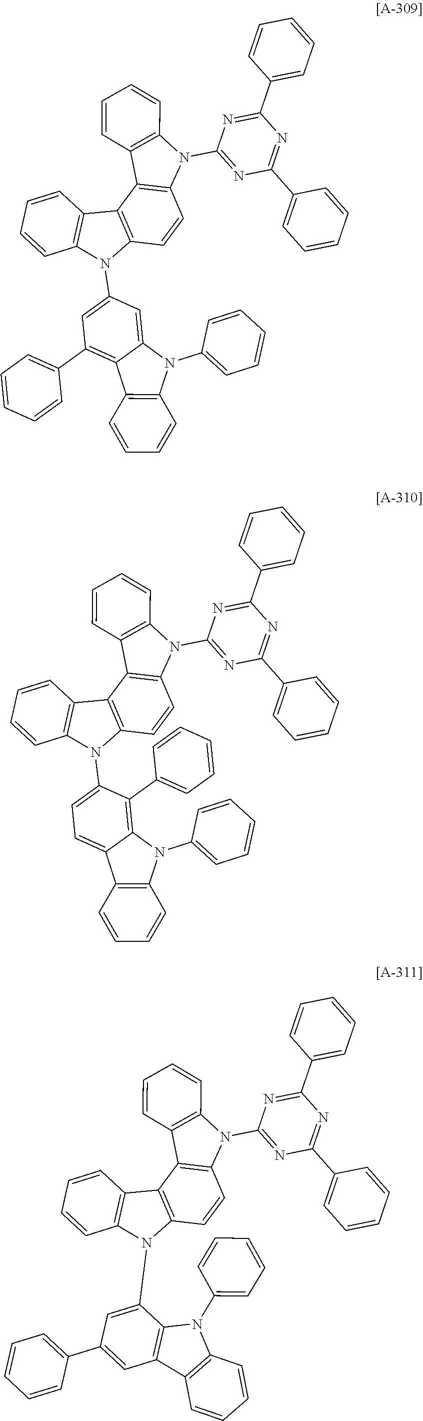 [SMILES: c1ccc(-c2nc(-c3ccccc3)nc(-n3c4ccccc4c4c5c6ccccc6n(-c6cc(-c7ccccc7)c7c8ccccc8n(-c8ccccc8)c7c6)c5ccc43)n2)cc1 ; c1ccc(-c2nc(-c3ccccc3)nc(-n3c4ccccc4c4c5c6ccccc6n(-c6ccc7c8ccccc8n(-c8ccccc8)c7c6-c6ccccc6)c5ccc43)n2)cc1 ; c1ccc(-c2cc(-n3c4ccccc4c4c5c6ccccc6n(-c6nc(-c7ccccc7)nc(-c7ccccc7)n6)c5ccc43)c3c(c2)c2ccccc2n3-c2ccccc2)cc1]

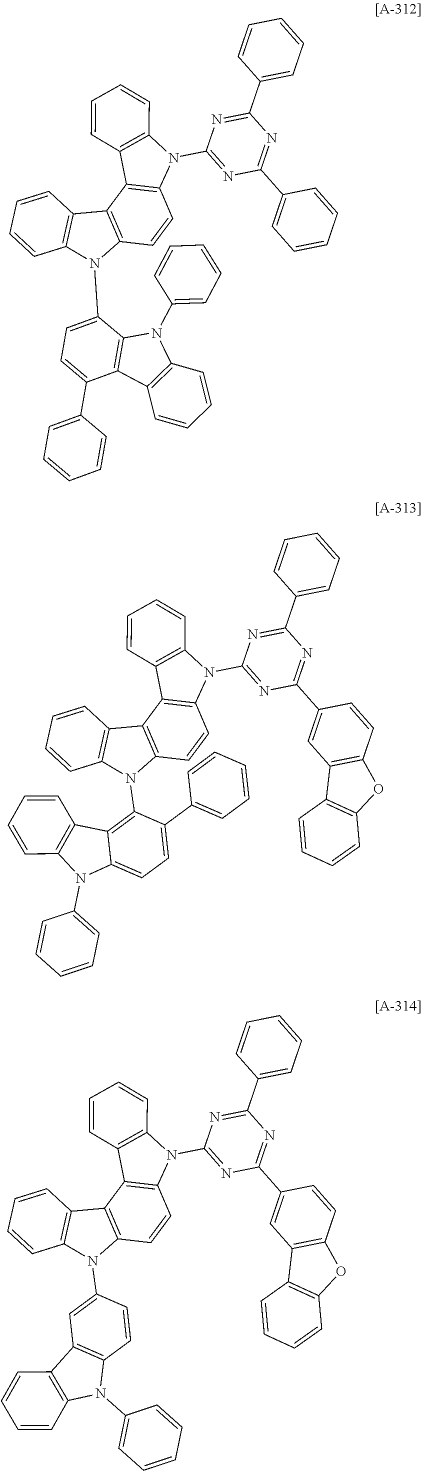 [SMILES: c1ccc(-c2nc(-c3ccccc3)nc(-n3c4ccccc4c4c5c6ccccc6n(-c6ccc(-c7ccccc7)c7c8ccccc8n(-c8ccccc8)c67)c5ccc43)n2)cc1 ; c1ccc(-c2nc(-c3ccc4oc5ccccc5c4c3)nc(-n3c4ccccc4c4c5c6ccccc6n(-c6c(-c7ccccc7)ccc7c6c6ccccc6n7-c6ccccc6)c5ccc43)n2)cc1 ; c1ccc(-c2nc(-c3ccc4oc5ccccc5c4c3)nc(-n3c4ccccc4c4c5c6ccccc6n(-c6ccc7c(c6)c6ccccc6n7-c6ccccc6)c5ccc43)n2)cc1]

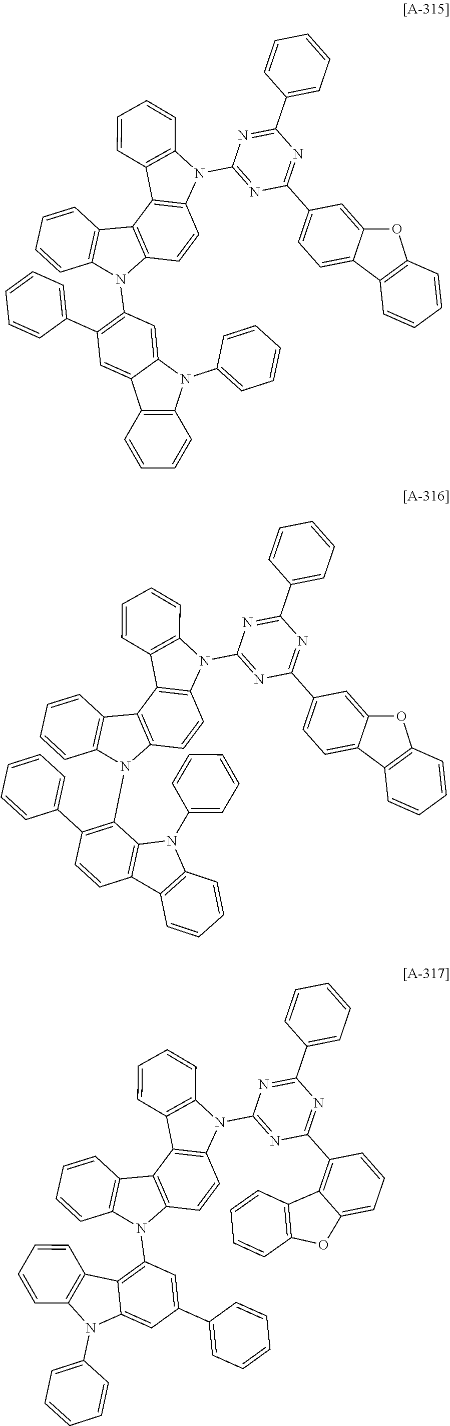 [SMILES: c1ccc(-c2nc(-c3ccc4c(c3)oc3ccccc34)nc(-n3c4ccccc4c4c5c6ccccc6n(-c6cc7c(cc6-c6ccccc6)c6ccccc6n7-c6ccccc6)c5ccc43)n2)cc1 ; c1ccc(-c2nc(-c3ccc4c(c3)oc3ccccc34)nc(-n3c4ccccc4c4c5c6ccccc6n(-c6c(-c7ccccc7)ccc7c8ccccc8n(-c8ccccc8)c67)c5ccc43)n2)cc1 ; c1ccc(-c2cc(-n3c4ccccc4c4c5c6ccccc6n(-c6nc(-c7ccccc7)nc(-c7cccc8oc9ccccc9c78)n6)c5ccc43)c3c4ccccc4n(-c4ccccc4)c3c2)cc1]

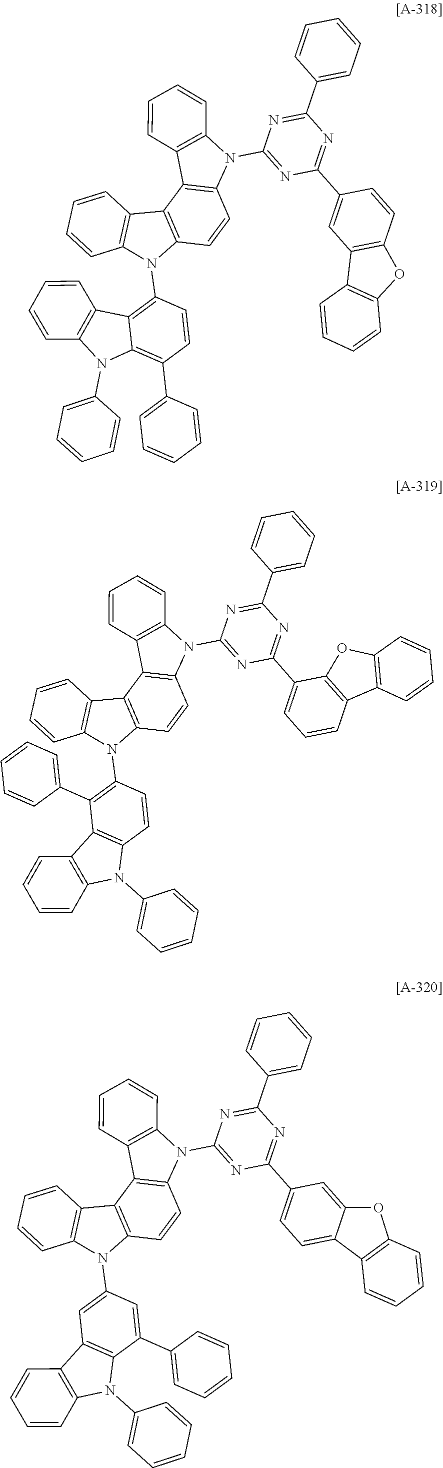 [SMILES: c1ccc(-c2nc(-c3ccc4oc5ccccc5c4c3)nc(-n3c4ccccc4c4c5c6ccccc6n(-c6ccc(-c7ccccc7)c7c6c6ccccc6n7-c6ccccc6)c5ccc43)n2)cc1 ; c1ccc(-c2nc(-c3cccc4c3oc3ccccc34)nc(-n3c4ccccc4c4c5c6ccccc6n(-c6ccc7c(c6-c6ccccc6)c6ccccc6n7-c6ccccc6)c5ccc43)n2)cc1 ; c1ccc(-c2nc(-c3ccc4c(c3)oc3ccccc34)nc(-n3c4ccccc4c4c5c6ccccc6n(-c6cc(-c7ccccc7)c7c(c6)c6ccccc6n7-c6ccccc6)c5ccc43)n2)cc1]

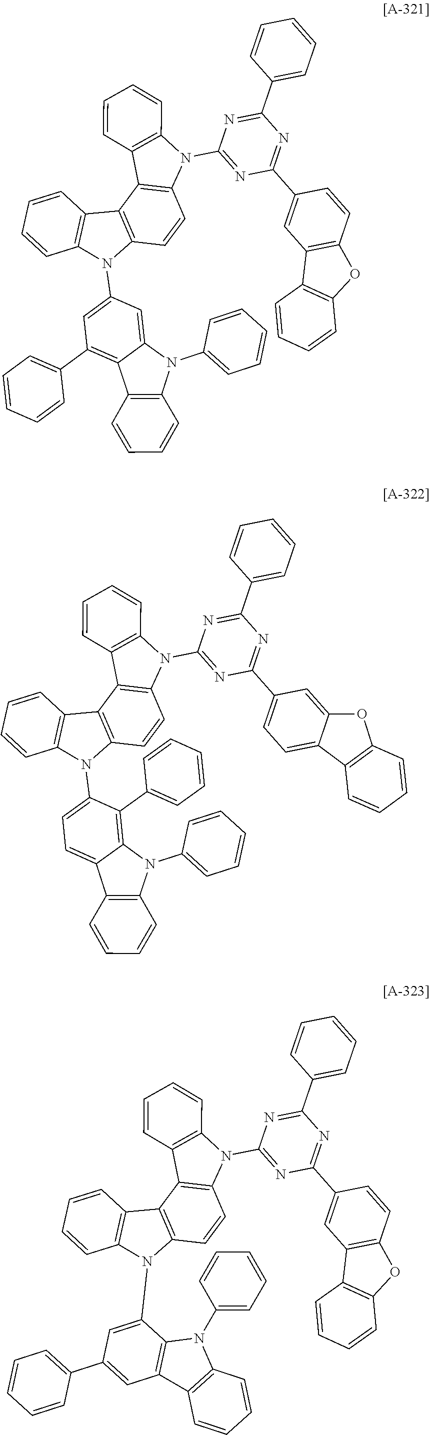 [SMILES: c1ccc(-c2nc(-c3ccc4oc5ccccc5c4c3)nc(-n3c4ccccc4c4c5c6ccccc6n(-c6cc(-c7ccccc7)c7c8ccccc8n(-c8ccccc8)c7c6)c5ccc43)n2)cc1 ; c1ccc(-c2nc(-c3ccc4c(c3)oc3ccccc34)nc(-n3c4ccccc4c4c5c6ccccc6n(-c6ccc7c8ccccc8n(-c8ccccc8)c7c6-c6ccccc6)c5ccc43)n2)cc1 ; c1ccc(-c2cc(-n3c4ccccc4c4c5c6ccccc6n(-c6nc(-c7ccccc7)nc(-c7ccc8oc9ccccc9c8c7)n6)c5ccc43)c3c(c2)c2ccccc2n3-c2ccccc2)cc1]

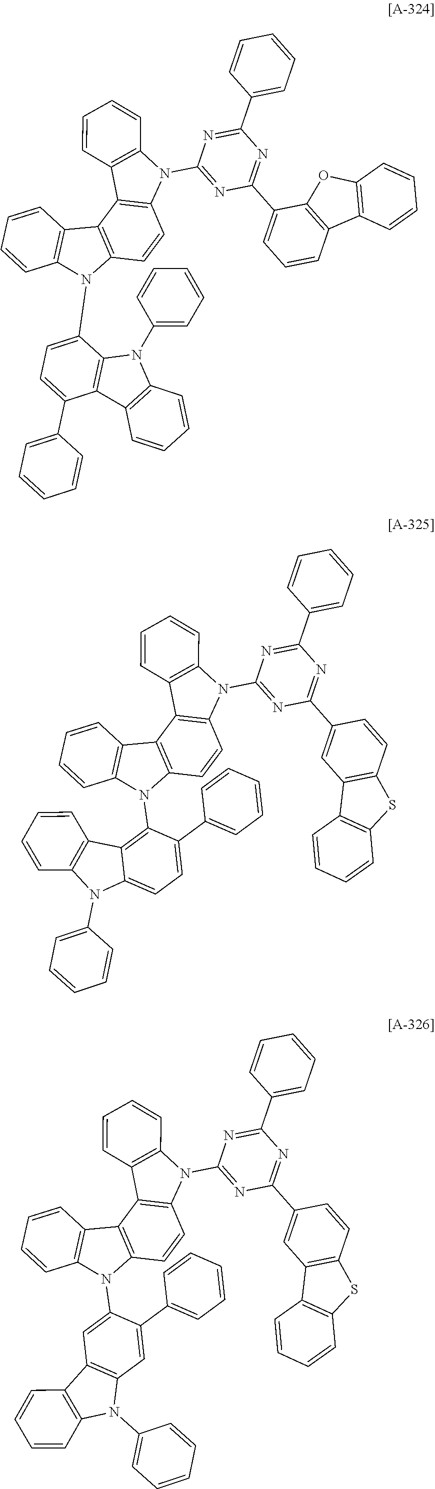 [SMILES: c1ccc(-c2nc(-c3cccc4c3oc3ccccc34)nc(-n3c4ccccc4c4c5c6ccccc6n(-c6ccc(-c7ccccc7)c7c8ccccc8n(-c8ccccc8)c67)c5ccc43)n2)cc1 ; c1ccc(-c2nc(-c3ccc4sc5ccccc5c4c3)nc(-n3c4ccccc4c4c5c6ccccc6n(-c6c(-c7ccccc7)ccc7c6c6ccccc6n7-c6ccccc6)c5ccc43)n2)cc1 ; c1ccc(-c2nc(-c3ccc4sc5ccccc5c4c3)nc(-n3c4ccccc4c4c5c6ccccc6n(-c6cc7c8ccccc8n(-c8ccccc8)c7cc6-c6ccccc6)c5ccc43)n2)cc1]

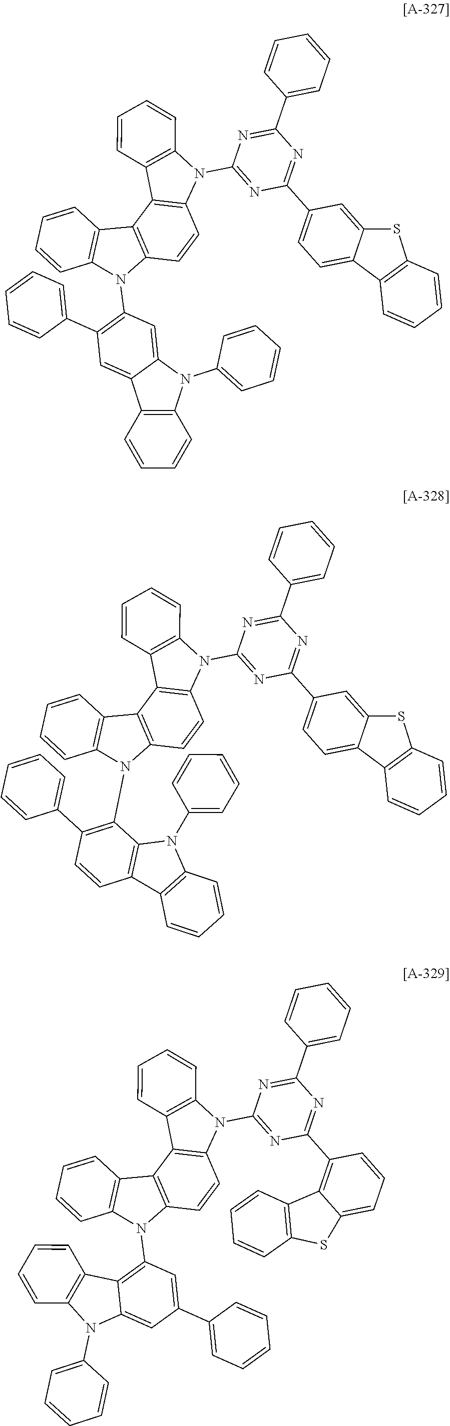 [SMILES: c1ccc(-c2nc(-c3ccc4c(c3)sc3ccccc34)nc(-n3c4ccccc4c4c5c6ccccc6n(-c6cc7c(cc6-c6ccccc6)c6ccccc6n7-c6ccccc6)c5ccc43)n2)cc1 ; c1ccc(-c2nc(-c3ccc4c(c3)sc3ccccc34)nc(-n3c4ccccc4c4c5c6ccccc6n(-c6c(-c7ccccc7)ccc7c8ccccc8n(-c8ccccc8)c67)c5ccc43)n2)cc1 ; c1ccc(-c2cc(-n3c4ccccc4c4c5c6ccccc6n(-c6nc(-c7ccccc7)nc(-c7cccc8sc9ccccc9c78)n6)c5ccc43)c3c4ccccc4n(-c4ccccc4)c3c2)cc1]

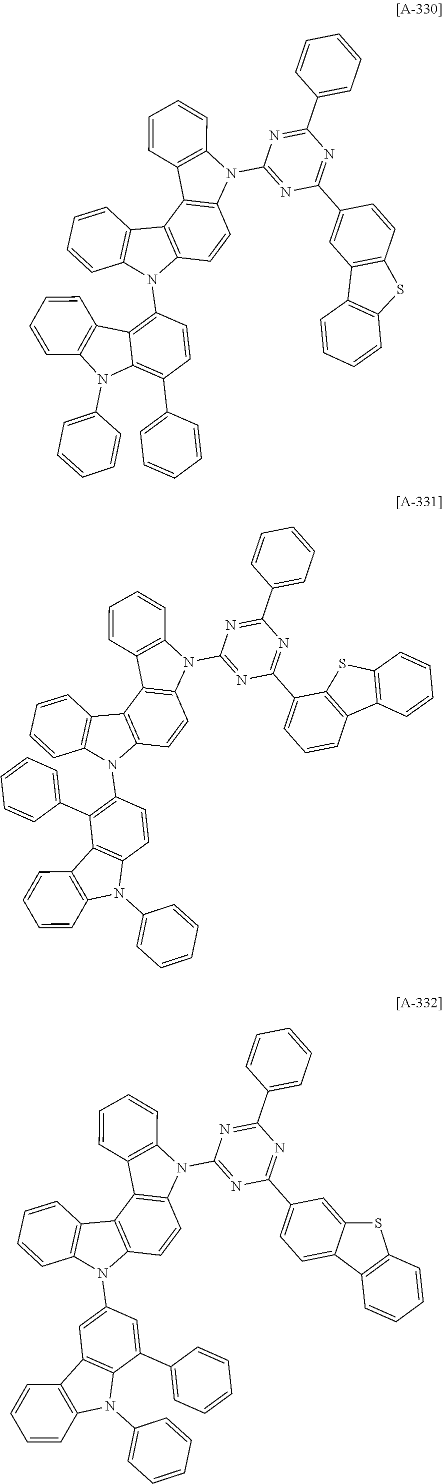 [SMILES: c1ccc(-c2nc(-c3ccc4sc5ccccc5c4c3)nc(-n3c4ccccc4c4c5c6ccccc6n(-c6ccc(-c7ccccc7)c7c6c6ccccc6n7-c6ccccc6)c5ccc43)n2)cc1 ; c1ccc(-c2nc(-c3cccc4c3sc3ccccc34)nc(-n3c4ccccc4c4c5c6ccccc6n(-c6ccc7c(c6-c6ccccc6)c6ccccc6n7-c6ccccc6)c5ccc43)n2)cc1 ; c1ccc(-c2nc(-c3ccc4c(c3)sc3ccccc34)nc(-n3c4ccccc4c4c5c6ccccc6n(-c6cc(-c7ccccc7)c7c(c6)c6ccccc6n7-c6ccccc6)c5ccc43)n2)cc1]

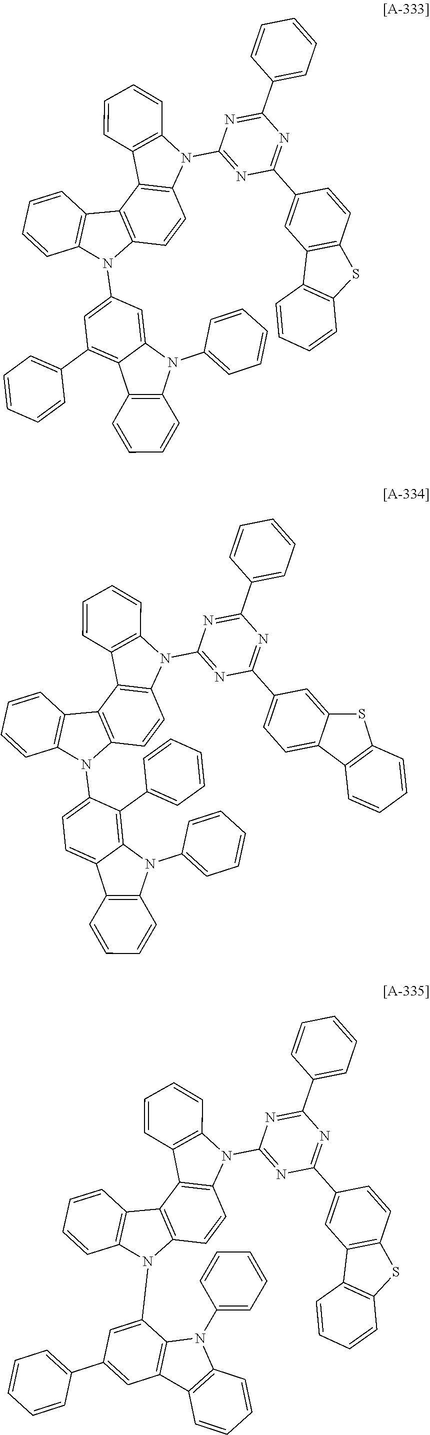 [SMILES: c1ccc(-c2nc(-c3ccc4sc5ccccc5c4c3)nc(-n3c4ccccc4c4c5c6ccccc6n(-c6cc(-c7ccccc7)c7c8ccccc8n(-c8ccccc8)c7c6)c5ccc43)n2)cc1 ; c1ccc(-c2nc(-c3ccc4c(c3)sc3ccccc34)nc(-n3c4ccccc4c4c5c6ccccc6n(-c6ccc7c8ccccc8n(-c8ccccc8)c7c6-c6ccccc6)c5ccc43)n2)cc1 ; c1ccc(-c2cc(-n3c4ccccc4c4c5c6ccccc6n(-c6nc(-c7ccccc7)nc(-c7ccc8sc9ccccc9c8c7)n6)c5ccc43)c3c(c2)c2ccccc2n3-c2ccccc2)cc1]

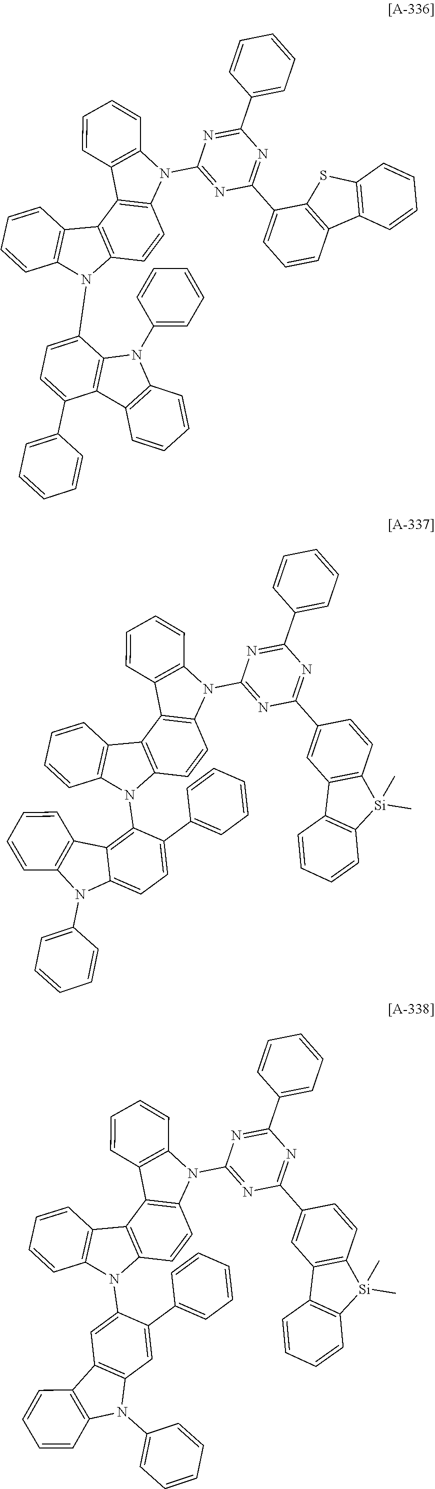 [SMILES: c1ccc(-c2nc(-c3cccc4c3sc3ccccc34)nc(-n3c4ccccc4c4c5c6ccccc6n(-c6ccc(-c7ccccc7)c7c8ccccc8n(-c8ccccc8)c67)c5ccc43)n2)cc1 ; C[Si]1(C)c2ccccc2-c2cc(-c3nc(-c4ccccc4)nc(-n4c5ccccc5c5c6c7ccccc7n(-c7c(-c8ccccc8)ccc8c7c7ccccc7n8-c7ccccc7)c6ccc54)n3)ccc21 ; C[Si]1(C)c2ccccc2-c2cc(-c3nc(-c4ccccc4)nc(-n4c5ccccc5c5c6c7ccccc7n(-c7cc8c9ccccc9n(-c9ccccc9)c8cc7-c7ccccc7)c6ccc54)n3)ccc21]

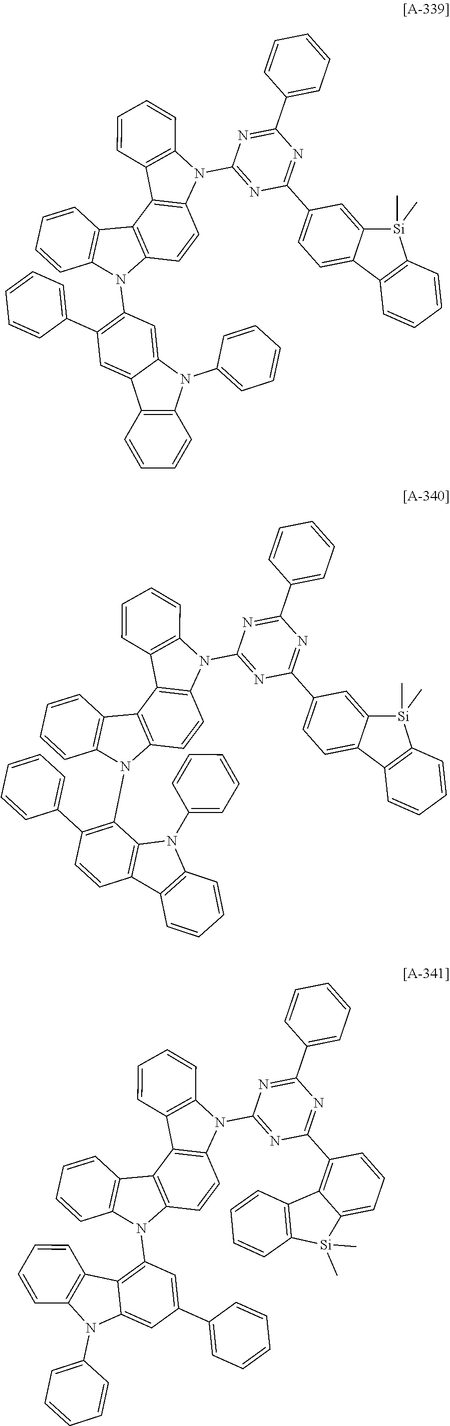 [SMILES: C[Si]1(C)c2ccccc2-c2ccc(-c3nc(-c4ccccc4)nc(-n4c5ccccc5c5c6c7ccccc7n(-c7cc8c(cc7-c7ccccc7)c7ccccc7n8-c7ccccc7)c6ccc54)n3)cc21 ; C[Si]1(C)c2ccccc2-c2ccc(-c3nc(-c4ccccc4)nc(-n4c5ccccc5c5c6c7ccccc7n(-c7c(-c8ccccc8)ccc8c9ccccc9n(-c9ccccc9)c78)c6ccc54)n3)cc21 ; C[Si]1(C)c2ccccc2-c2c(-c3nc(-c4ccccc4)nc(-n4c5ccccc5c5c6c7ccccc7n(-c7cc(-c8ccccc8)cc8c7c7ccccc7n8-c7ccccc7)c6ccc54)n3)cccc21]

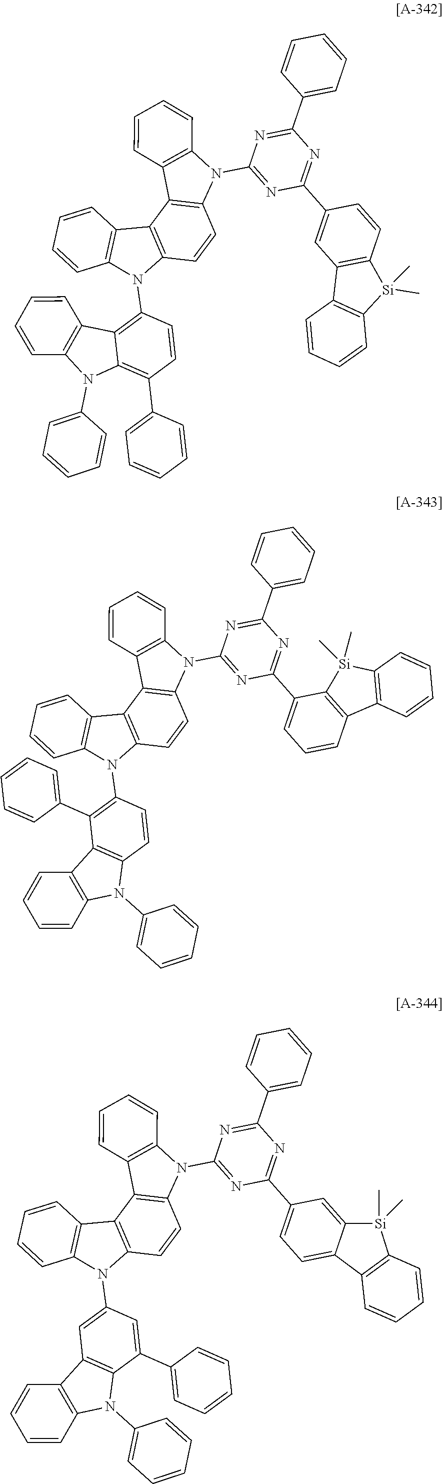 [SMILES: C[Si]1(C)c2ccccc2-c2cc(-c3nc(-c4ccccc4)nc(-n4c5ccccc5c5c6c7ccccc7n(-c7ccc(-c8ccccc8)c8c7c7ccccc7n8-c7ccccc7)c6ccc54)n3)ccc21 ; C[Si]1(C)c2ccccc2-c2cccc(-c3nc(-c4ccccc4)nc(-n4c5ccccc5c5c6c7ccccc7n(-c7ccc8c(c7-c7ccccc7)c7ccccc7n8-c7ccccc7)c6ccc54)n3)c21 ; C[Si]1(C)c2ccccc2-c2ccc(-c3nc(-c4ccccc4)nc(-n4c5ccccc5c5c6c7ccccc7n(-c7cc(-c8ccccc8)c8c(c7)c7ccccc7n8-c7ccccc7)c6ccc54)n3)cc21]

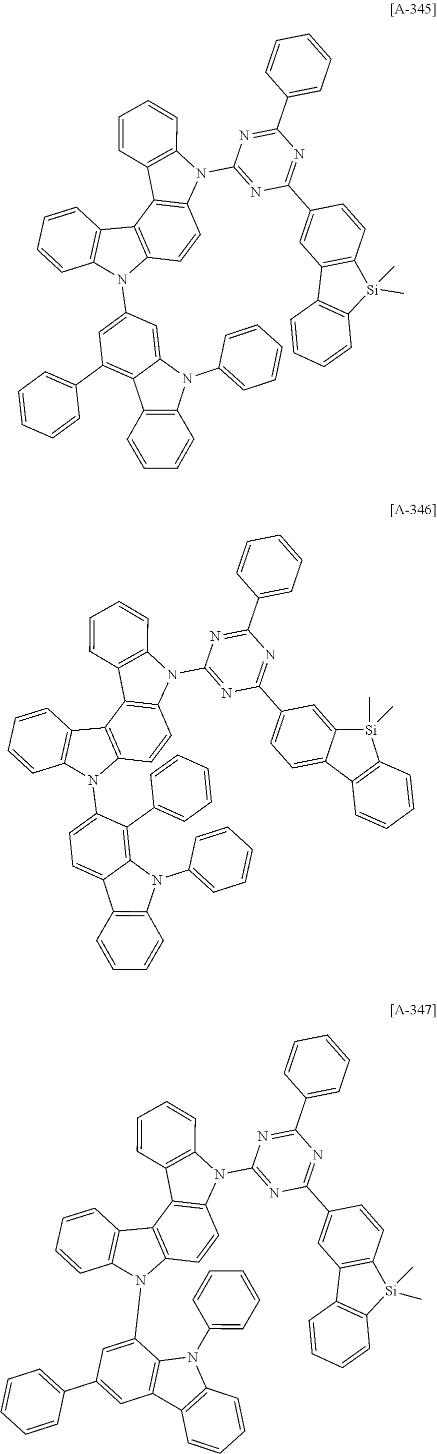 [SMILES: C[Si]1(C)c2ccccc2-c2cc(-c3nc(-c4ccccc4)nc(-n4c5ccccc5c5c6c7ccccc7n(-c7cc(-c8ccccc8)c8c9ccccc9n(-c9ccccc9)c8c7)c6ccc54)n3)ccc21 ; C[Si]1(C)c2ccccc2-c2ccc(-c3nc(-c4ccccc4)nc(-n4c5ccccc5c5c6c7ccccc7n(-c7ccc8c9ccccc9n(-c9ccccc9)c8c7-c7ccccc7)c6ccc54)n3)cc21 ; C[Si]1(C)c2ccccc2-c2cc(-c3nc(-c4ccccc4)nc(-n4c5ccccc5c5c6c7ccccc7n(-c7cc(-c8ccccc8)cc8c9ccccc9n(-c9ccccc9)c78)c6ccc54)n3)ccc21]

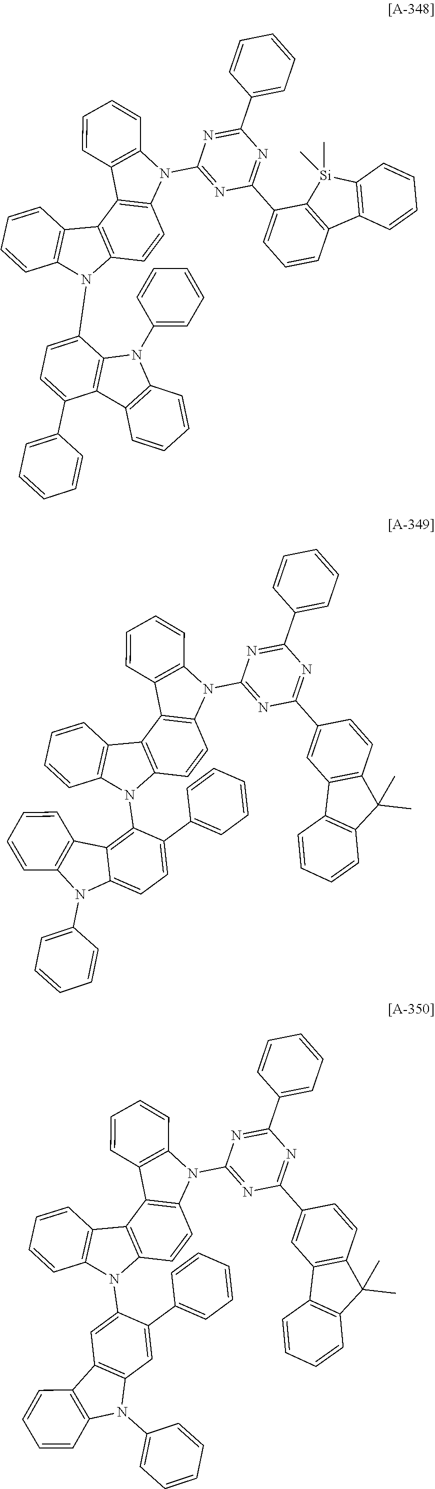 [SMILES: C[Si]1(C)c2ccccc2-c2cccc(-c3nc(-c4ccccc4)nc(-n4c5ccccc5c5c6c7ccccc7n(-c7ccc(-c8ccccc8)c8c9ccccc9n(-c9ccccc9)c78)c6ccc54)n3)c21 ; CC1(C)c2ccccc2-c2cc(-c3nc(-c4ccccc4)nc(-n4c5ccccc5c5c6c7ccccc7n(-c7c(-c8ccccc8)ccc8c7c7ccccc7n8-c7ccccc7)c6ccc54)n3)ccc21 ; CC1(C)c2ccccc2-c2cc(-c3nc(-c4ccccc4)nc(-n4c5ccccc5c5c6c7ccccc7n(-c7cc8c9ccccc9n(-c9ccccc9)c8cc7-c7ccccc7)c6ccc54)n3)ccc21]

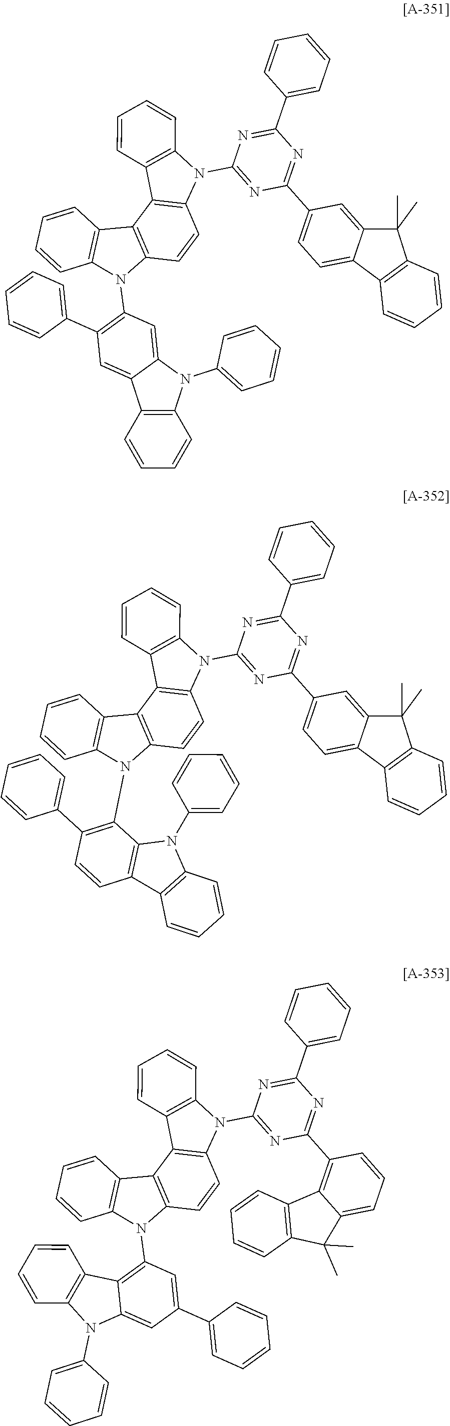 [SMILES: CC1(C)c2ccccc2-c2ccc(-c3nc(-c4ccccc4)nc(-n4c5ccccc5c5c6c7ccccc7n(-c7cc8c(cc7-c7ccccc7)c7ccccc7n8-c7ccccc7)c6ccc54)n3)cc21 ; CC1(C)c2ccccc2-c2ccc(-c3nc(-c4ccccc4)nc(-n4c5ccccc5c5c6c7ccccc7n(-c7c(-c8ccccc8)ccc8c9ccccc9n(-c9ccccc9)c78)c6ccc54)n3)cc21 ; CC1(C)c2ccccc2-c2c(-c3nc(-c4ccccc4)nc(-n4c5ccccc5c5c6c7ccccc7n(-c7cc(-c8ccccc8)cc8c7c7ccccc7n8-c7ccccc7)c6ccc54)n3)cccc21]

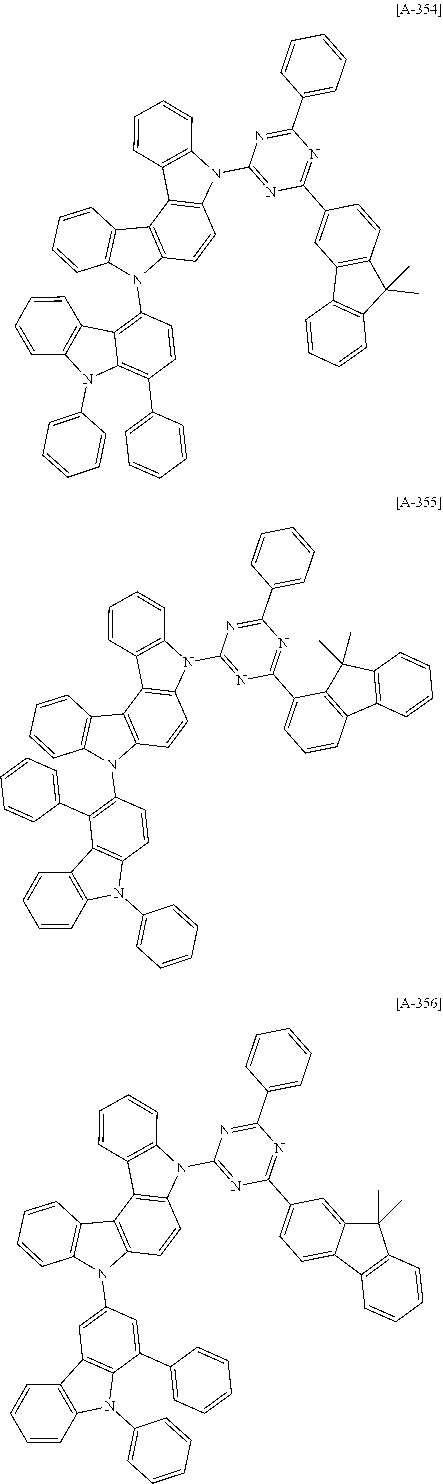 [SMILES: CC1(C)c2ccccc2-c2cc(-c3nc(-c4ccccc4)nc(-n4c5ccccc5c5c6c7ccccc7n(-c7ccc(-c8ccccc8)c8c7c7ccccc7n8-c7ccccc7)c6ccc54)n3)ccc21 ; CC1(C)c2ccccc2-c2cccc(-c3nc(-c4ccccc4)nc(-n4c5ccccc5c5c6c7ccccc7n(-c7ccc8c(c7-c7ccccc7)c7ccccc7n8-c7ccccc7)c6ccc54)n3)c21 ; CC1(C)c2ccccc2-c2ccc(-c3nc(-c4ccccc4)nc(-n4c5ccccc5c5c6c7ccccc7n(-c7cc(-c8ccccc8)c8c(c7)c7ccccc7n8-c7ccccc7)c6ccc54)n3)cc21]

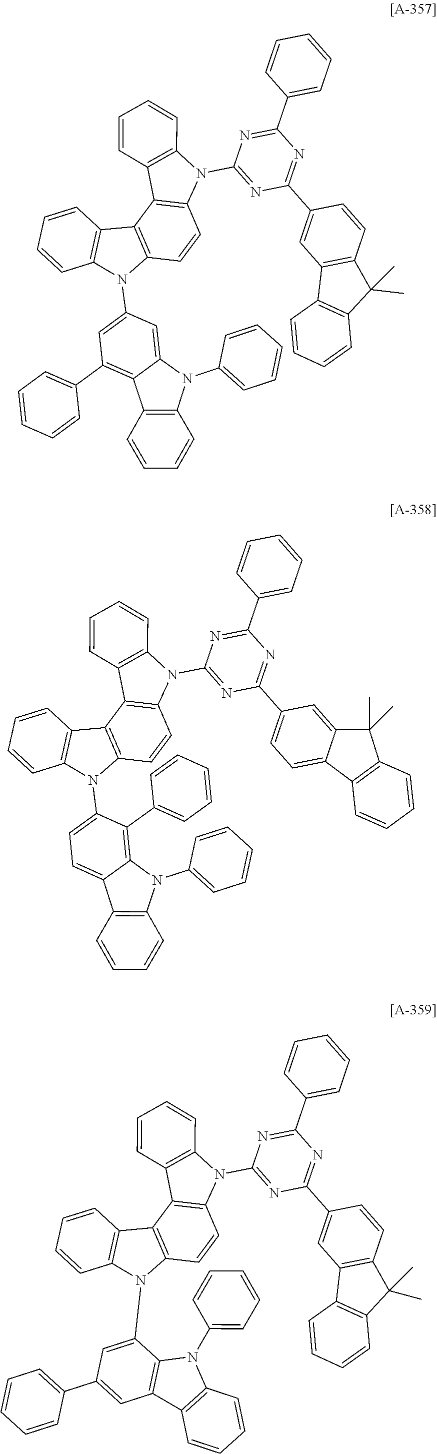 [SMILES: CC1(C)c2ccccc2-c2cc(-c3nc(-c4ccccc4)nc(-n4c5ccccc5c5c6c7ccccc7n(-c7cc(-c8ccccc8)c8c9ccccc9n(-c9ccccc9)c8c7)c6ccc54)n3)ccc21 ; CC1(C)c2ccccc2-c2ccc(-c3nc(-c4ccccc4)nc(-n4c5ccccc5c5c6c7ccccc7n(-c7ccc8c9ccccc9n(-c9ccccc9)c8c7-c7ccccc7)c6ccc54)n3)cc21 ; CC1(C)c2ccccc2-c2cc(-c3nc(-c4ccccc4)nc(-n4c5ccccc5c5c6c7ccccc7n(-c7cc(-c8ccccc8)cc8c9ccccc9n(-c9ccccc9)c78)c6ccc54)n3)ccc21]

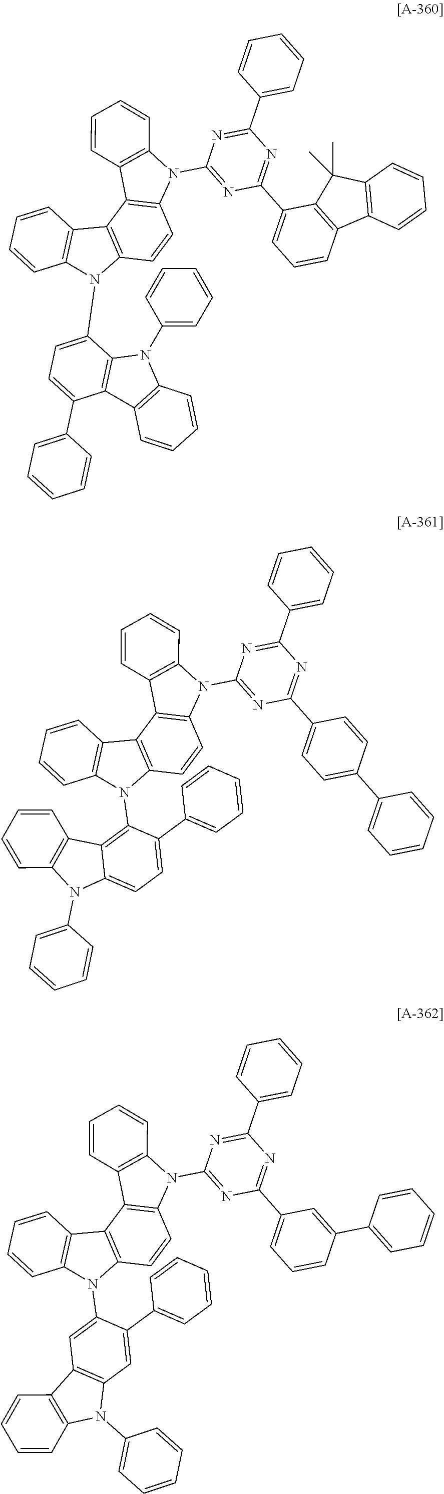 [SMILES: CC1(C)c2ccccc2-c2cccc(-c3nc(-c4ccccc4)nc(-n4c5ccccc5c5c6c7ccccc7n(-c7ccc(-c8ccccc8)c8c9ccccc9n(-c9ccccc9)c78)c6ccc54)n3)c21 ; c1ccc(-c2ccc(-c3nc(-c4ccccc4)nc(-n4c5ccccc5c5c6c7ccccc7n(-c7c(-c8ccccc8)ccc8c7c7ccccc7n8-c7ccccc7)c6ccc54)n3)cc2)cc1 ; c1ccc(-c2cccc(-c3nc(-c4ccccc4)nc(-n4c5ccccc5c5c6c7ccccc7n(-c7cc8c9ccccc9n(-c9ccccc9)c8cc7-c7ccccc7)c6ccc54)n3)c2)cc1]

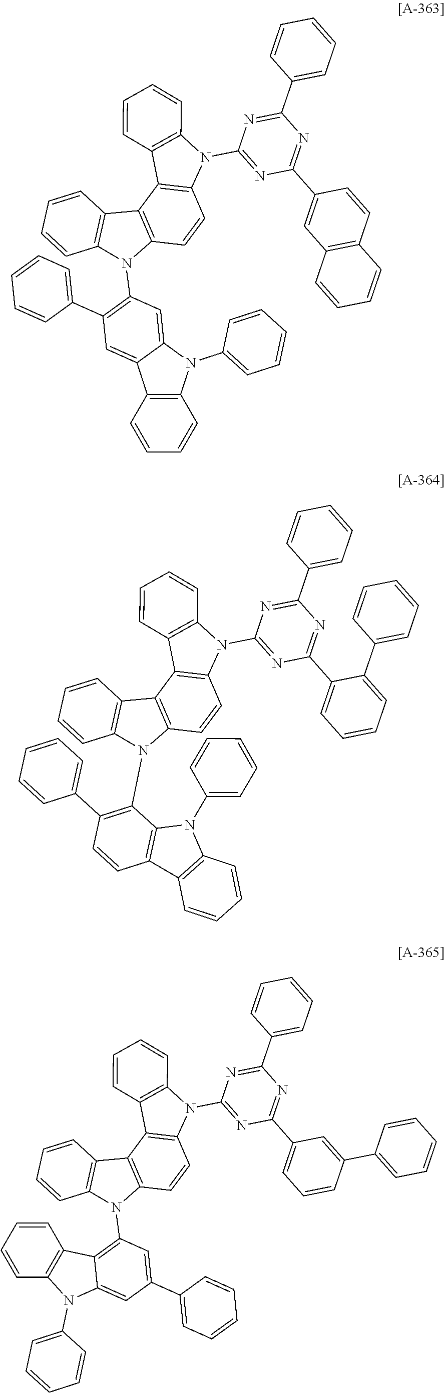 [SMILES: c1ccc(-c2nc(-c3ccc4ccccc4c3)nc(-n3c4ccccc4c4c5c6ccccc6n(-c6cc7c(cc6-c6ccccc6)c6ccccc6n7-c6ccccc6)c5ccc43)n2)cc1 ; c1ccc(-c2nc(-c3ccccc3-c3ccccc3)nc(-n3c4ccccc4c4c5c6ccccc6n(-c6c(-c7ccccc7)ccc7c8ccccc8n(-c8ccccc8)c67)c5ccc43)n2)cc1 ; c1ccc(-c2cccc(-c3nc(-c4ccccc4)nc(-n4c5ccccc5c5c6c7ccccc7n(-c7cc(-c8ccccc8)cc8c7c7ccccc7n8-c7ccccc7)c6ccc54)n3)c2)cc1]

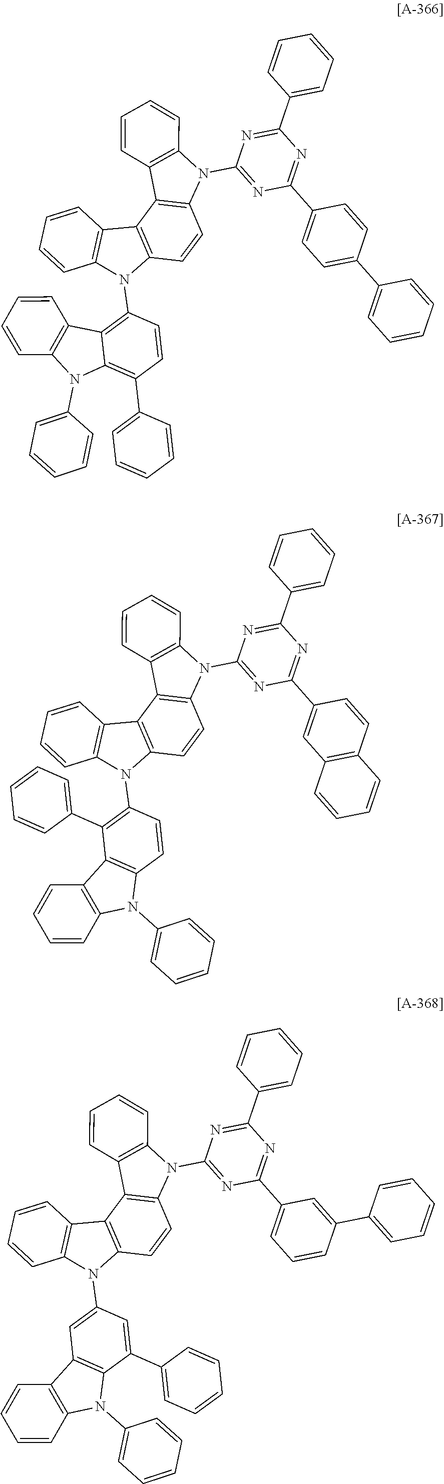 [SMILES: c1ccc(-c2ccc(-c3nc(-c4ccccc4)nc(-n4c5ccccc5c5c6c7ccccc7n(-c7ccc(-c8ccccc8)c8c7c7ccccc7n8-c7ccccc7)c6ccc54)n3)cc2)cc1 ; c1ccc(-c2nc(-c3ccc4ccccc4c3)nc(-n3c4ccccc4c4c5c6ccccc6n(-c6ccc7c(c6-c6ccccc6)c6ccccc6n7-c6ccccc6)c5ccc43)n2)cc1 ; c1ccc(-c2cccc(-c3nc(-c4ccccc4)nc(-n4c5ccccc5c5c6c7ccccc7n(-c7cc(-c8ccccc8)c8c(c7)c7ccccc7n8-c7ccccc7)c6ccc54)n3)c2)cc1]

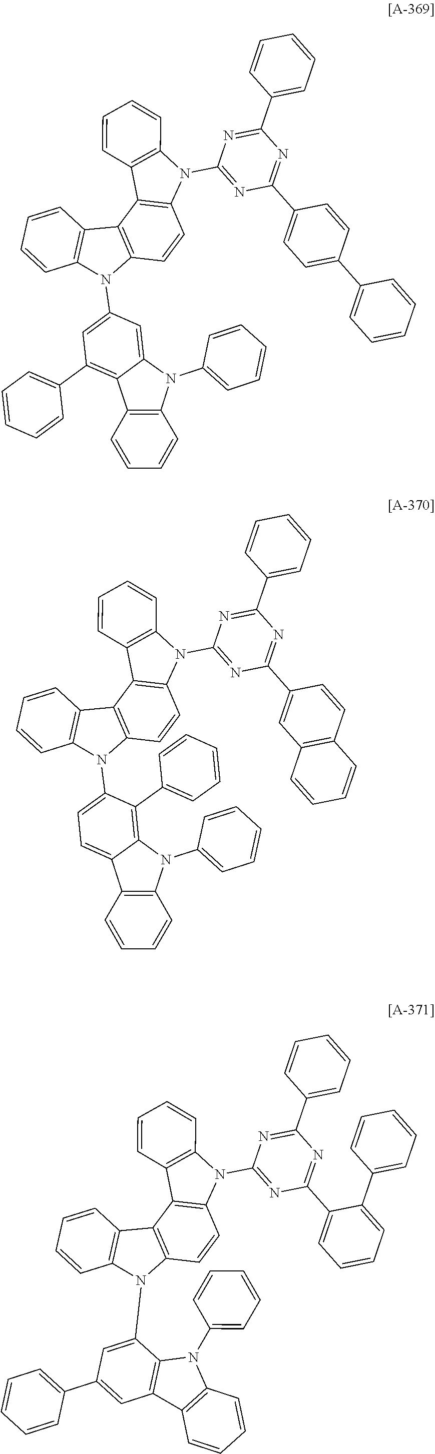 [SMILES: c1ccc(-c2ccc(-c3nc(-c4ccccc4)nc(-n4c5ccccc5c5c6c7ccccc7n(-c7cc(-c8ccccc8)c8c9ccccc9n(-c9ccccc9)c8c7)c6ccc54)n3)cc2)cc1 ; c1ccc(-c2nc(-c3ccc4ccccc4c3)nc(-n3c4ccccc4c4c5c6ccccc6n(-c6ccc7c8ccccc8n(-c8ccccc8)c7c6-c6ccccc6)c5ccc43)n2)cc1 ; c1ccc(-c2cc(-n3c4ccccc4c4c5c6ccccc6n(-c6nc(-c7ccccc7)nc(-c7ccccc7-c7ccccc7)n6)c5ccc43)c3c(c2)c2ccccc2n3-c2ccccc2)cc1]

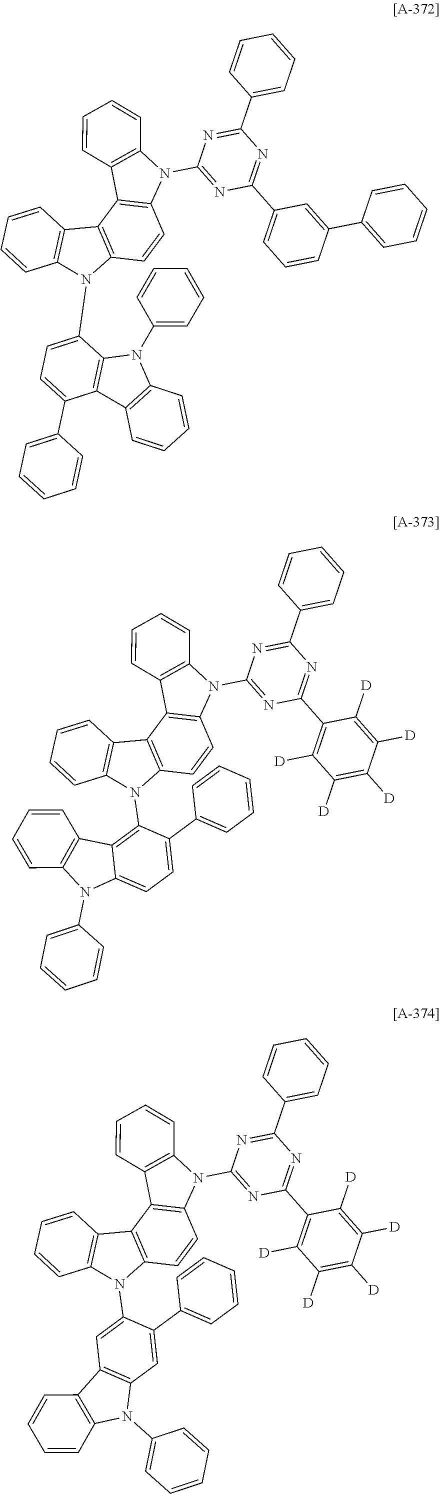 [SMILES: c1ccc(-c2cccc(-c3nc(-c4ccccc4)nc(-n4c5ccccc5c5c6c7ccccc7n(-c7ccc(-c8ccccc8)c8c9ccccc9n(-c9ccccc9)c78)c6ccc54)n3)c2)cc1 ; [2H]c1c([2H])c([2H])c(-c2nc(-c3ccccc3)nc(-n3c4ccccc4c4c5c6ccccc6n(-c6c(-c7ccccc7)ccc7c6c6ccccc6n7-c6ccccc6)c5ccc43)n2)c([2H])c1[2H] ; [2H]c1c([2H])c([2H])c(-c2nc(-c3ccccc3)nc(-n3c4ccccc4c4c5c6ccccc6n(-c6cc7c8ccccc8n(-c8ccccc8)c7cc6-c6ccccc6)c5ccc43)n2)c([2H])c1[2H]]

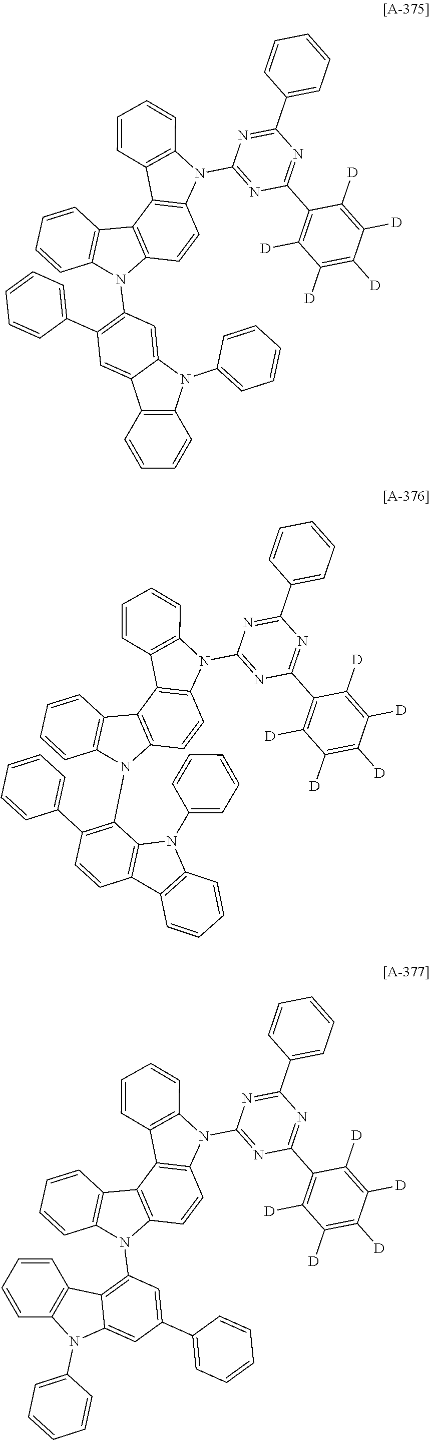 [SMILES: [2H]c1c([2H])c([2H])c(-c2nc(-c3ccccc3)nc(-n3c4ccccc4c4c5c6ccccc6n(-c6cc7c(cc6-c6ccccc6)c6ccccc6n7-c6ccccc6)c5ccc43)n2)c([2H])c1[2H] ; [2H]c1c([2H])c([2H])c(-c2nc(-c3ccccc3)nc(-n3c4ccccc4c4c5c6ccccc6n(-c6c(-c7ccccc7)ccc7c8ccccc8n(-c8ccccc8)c67)c5ccc43)n2)c([2H])c1[2H] ; [2H]c1c([2H])c([2H])c(-c2nc(-c3ccccc3)nc(-n3c4ccccc4c4c5c6ccccc6n(-c6cc(-c7ccccc7)cc7c6c6ccccc6n7-c6ccccc6)c5ccc43)n2)c([2H])c1[2H]]

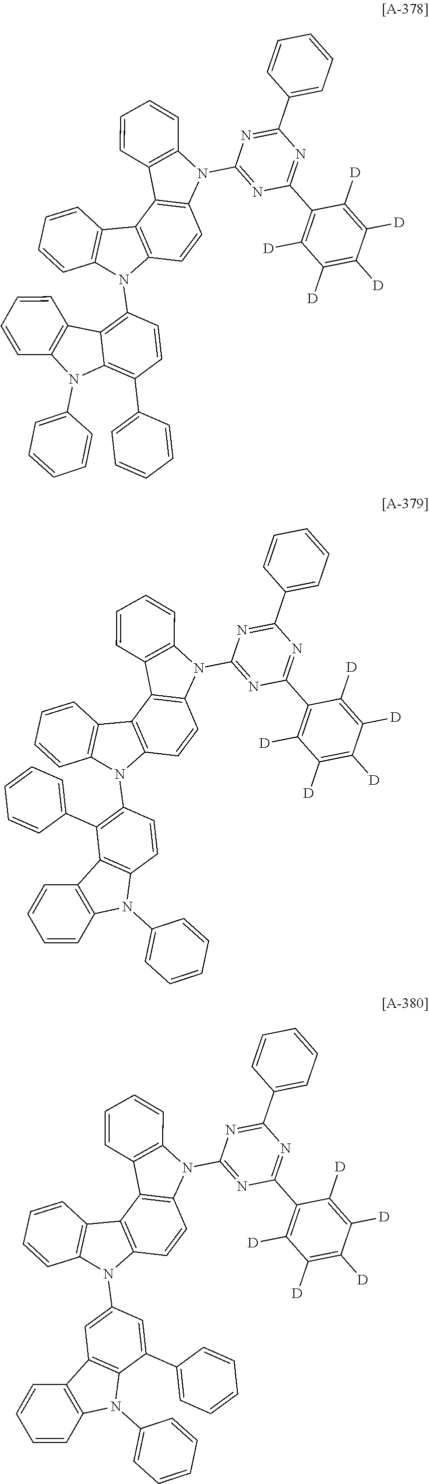 [SMILES: [2H]c1c([2H])c([2H])c(-c2nc(-c3ccccc3)nc(-n3c4ccccc4c4c5c6ccccc6n(-c6ccc(-c7ccccc7)c7c6c6ccccc6n7-c6ccccc6)c5ccc43)n2)c([2H])c1[2H] ; [2H]c1c([2H])c([2H])c(-c2nc(-c3ccccc3)nc(-n3c4ccccc4c4c5c6ccccc6n(-c6ccc7c(c6-c6ccccc6)c6ccccc6n7-c6ccccc6)c5ccc43)n2)c([2H])c1[2H] ; [2H]c1c([2H])c([2H])c(-c2nc(-c3ccccc3)nc(-n3c4ccccc4c4c5c6ccccc6n(-c6cc(-c7ccccc7)c7c(c6)c6ccccc6n7-c6ccccc6)c5ccc43)n2)c([2H])c1[2H]]

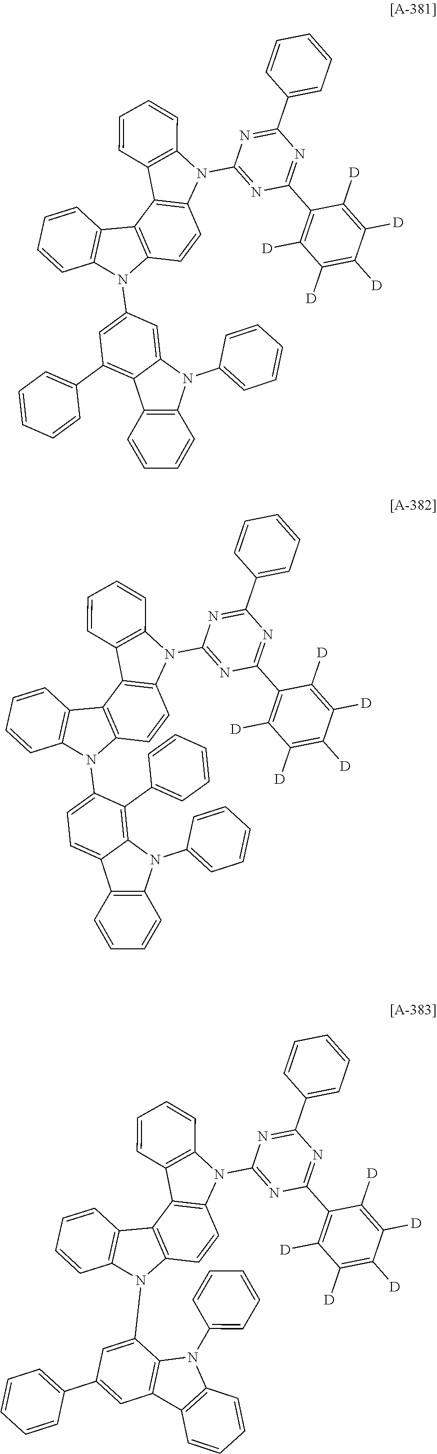 [SMILES: [2H]c1c([2H])c([2H])c(-c2nc(-c3ccccc3)nc(-n3c4ccccc4c4c5c6ccccc6n(-c6cc(-c7ccccc7)c7c8ccccc8n(-c8ccccc8)c7c6)c5ccc43)n2)c([2H])c1[2H] ; [2H]c1c([2H])c([2H])c(-c2nc(-c3ccccc3)nc(-n3c4ccccc4c4c5c6ccccc6n(-c6ccc7c8ccccc8n(-c8ccccc8)c7c6-c6ccccc6)c5ccc43)n2)c([2H])c1[2H] ; [2H]c1c([2H])c([2H])c(-c2nc(-c3ccccc3)nc(-n3c4ccccc4c4c5c6ccccc6n(-c6cc(-c7ccccc7)cc7c8ccccc8n(-c8ccccc8)c67)c5ccc43)n2)c([2H])c1[2H]]

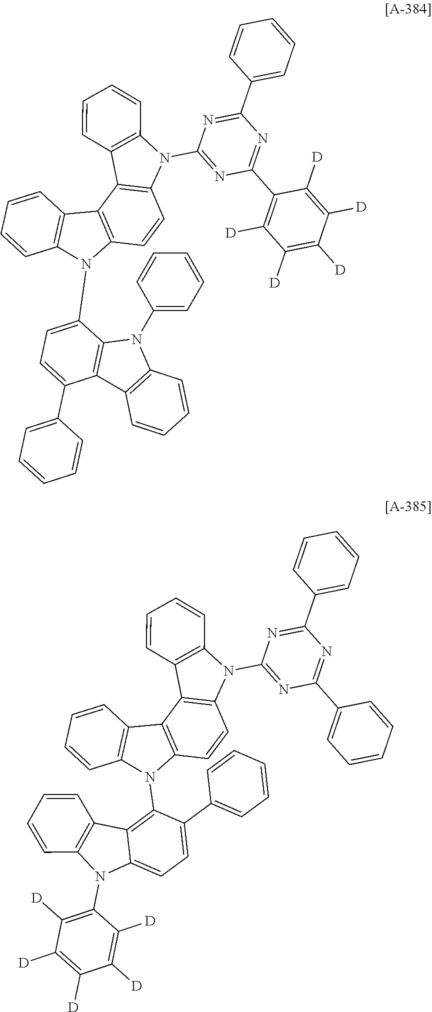 [SMILES: [2H]c1c([2H])c([2H])c(-c2nc(-c3ccccc3)nc(-n3c4ccccc4c4c5c6ccccc6n(-c6ccc(-c7ccccc7)c7c8ccccc8n(-c8ccccc8)c67)c5ccc43)n2)c([2H])c1[2H] ; [2H]c1c([2H])c([2H])c(-n2c3ccccc3c3c(-n4c5ccccc5c5c6c7ccccc7n(-c7nc(-c8ccccc8)nc(-c8ccccc8)n7)c6ccc54)c(-c4ccccc4)ccc32)c([2H])c1[2H]]

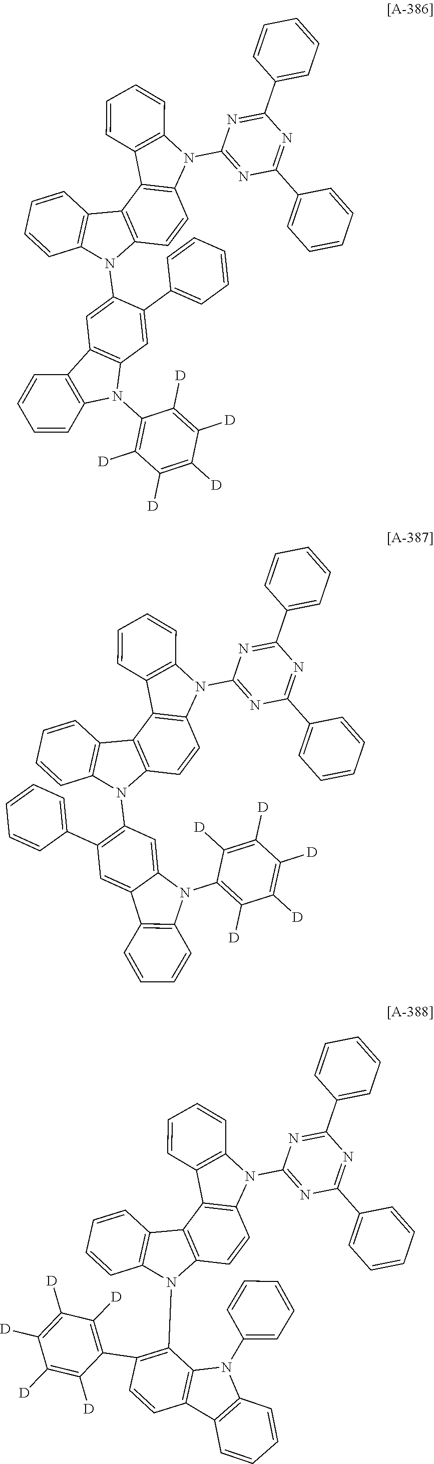 [SMILES: [2H]c1c([2H])c([2H])c(-n2c3ccccc3c3cc(-n4c5ccccc5c5c6c7ccccc7n(-c7nc(-c8ccccc8)nc(-c8ccccc8)n7)c6ccc54)c(-c4ccccc4)cc32)c([2H])c1[2H] ; [2H]c1c([2H])c([2H])c(-n2c3ccccc3c3cc(-c4ccccc4)c(-n4c5ccccc5c5c6c7ccccc7n(-c7nc(-c8ccccc8)nc(-c8ccccc8)n7)c6ccc54)cc32)c([2H])c1[2H] ; [2H]c1c([2H])c([2H])c(-c2ccc3c4ccccc4n(-c4ccccc4)c3c2-n2c3ccccc3c3c4c5ccccc5n(-c5nc(-c6ccccc6)nc(-c6ccccc6)n5)c4ccc32)c([2H])c1[2H]]

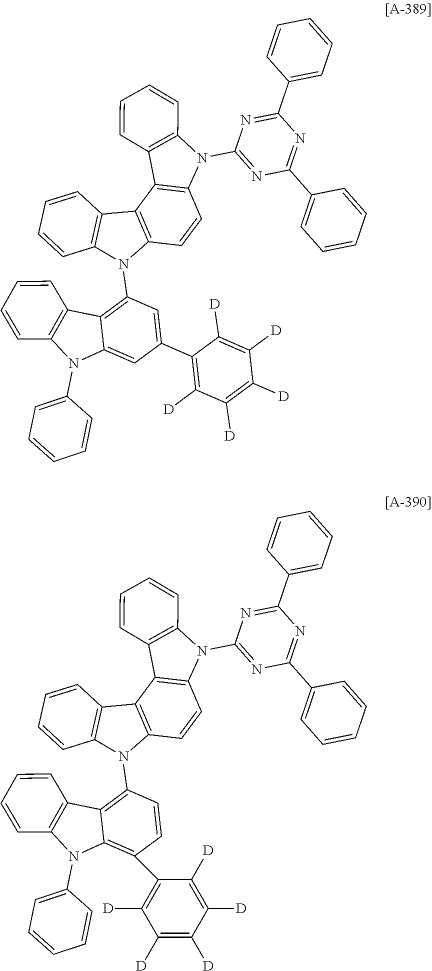 [SMILES: [2H]c1c([2H])c([2H])c(-c2cc(-n3c4ccccc4c4c5c6ccccc6n(-c6nc(-c7ccccc7)nc(-c7ccccc7)n6)c5ccc43)c3c4ccccc4n(-c4ccccc4)c3c2)c([2H])c1[2H] ; [2H]c1c([2H])c([2H])c(-c2ccc(-n3c4ccccc4c4c5c6ccccc6n(-c6nc(-c7ccccc7)nc(-c7ccccc7)n6)c5ccc43)c3c4ccccc4n(-c4ccccc4)c23)c([2H])c1[2H]]

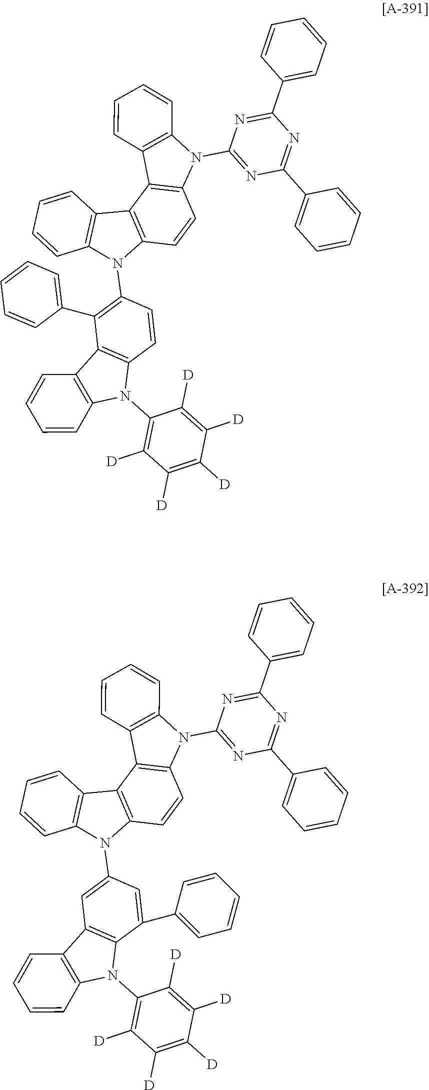 [SMILES: [2H]c1c([2H])c([2H])c(-n2c3ccccc3c3c(-c4ccccc4)c(-n4c5ccccc5c5c6c7ccccc7n(-c7nc(-c8ccccc8)nc(-c8ccccc8)n7)c6ccc54)ccc32)c([2H])c1[2H] ; [2H]c1c([2H])c([2H])c(-n2c3ccccc3c3cc(-n4c5ccccc5c5c6c7ccccc7n(-c7nc(-c8ccccc8)nc(-c8ccccc8)n7)c6ccc54)cc(-c4ccccc4)c32)c([2H])c1[2H]]

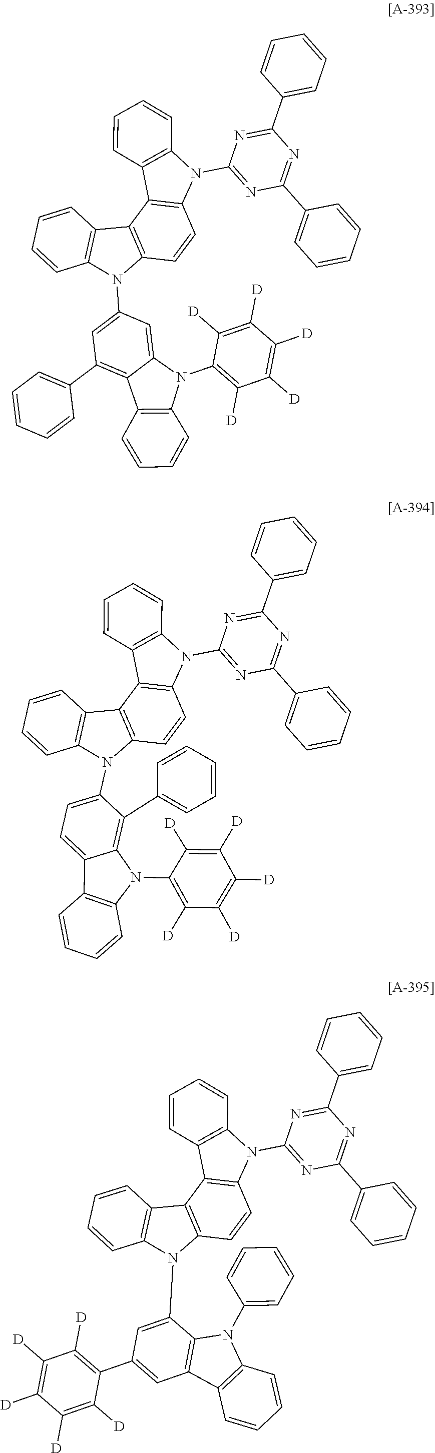 [SMILES: [2H]c1c([2H])c([2H])c(-n2c3ccccc3c3c(-c4ccccc4)cc(-n4c5ccccc5c5c6c7ccccc7n(-c7nc(-c8ccccc8)nc(-c8ccccc8)n7)c6ccc54)cc32)c([2H])c1[2H] ; [2H]c1c([2H])c([2H])c(-n2c3ccccc3c3ccc(-n4c5ccccc5c5c6c7ccccc7n(-c7nc(-c8ccccc8)nc(-c8ccccc8)n7)c6ccc54)c(-c4ccccc4)c32)c([2H])c1[2H] ; [2H]c1c([2H])c([2H])c(-c2cc(-n3c4ccccc4c4c5c6ccccc6n(-c6nc(-c7ccccc7)nc(-c7ccccc7)n6)c5ccc43)c3c(c2)c2ccccc2n3-c2ccccc2)c([2H])c1[2H]]

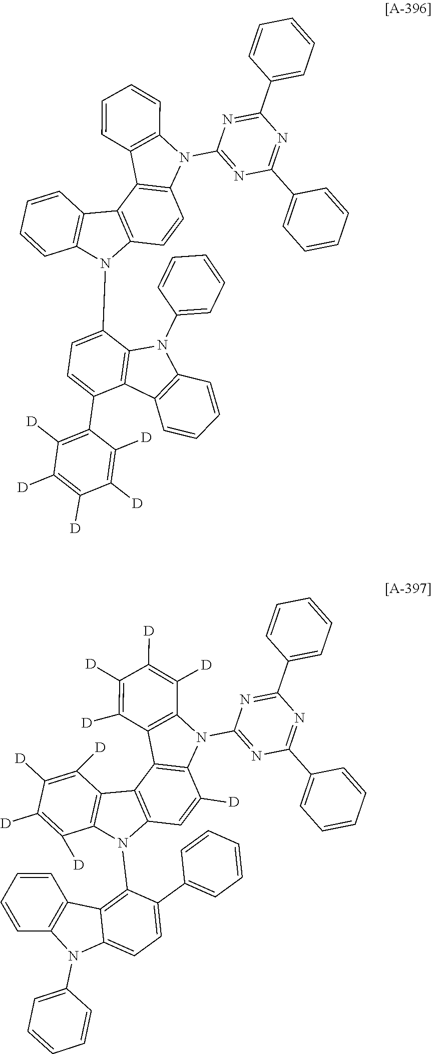 [SMILES: [2H]c1c([2H])c([2H])c(-c2ccc(-n3c4ccccc4c4c5c6ccccc6n(-c6nc(-c7ccccc7)nc(-c7ccccc7)n6)c5ccc43)c3c2c2ccccc2n3-c2ccccc2)c([2H])c1[2H] ; [2H]c1c([2H])c([2H])c2c(c1[2H])c1c3c4c([2H])c([2H])c([2H])c([2H])c4n(-c4nc(-c5ccccc5)nc(-c5ccccc5)n4)c3c([2H])cc1n2-c1c(-c2ccccc2)ccc2c1c1ccccc1n2-c1ccccc1]

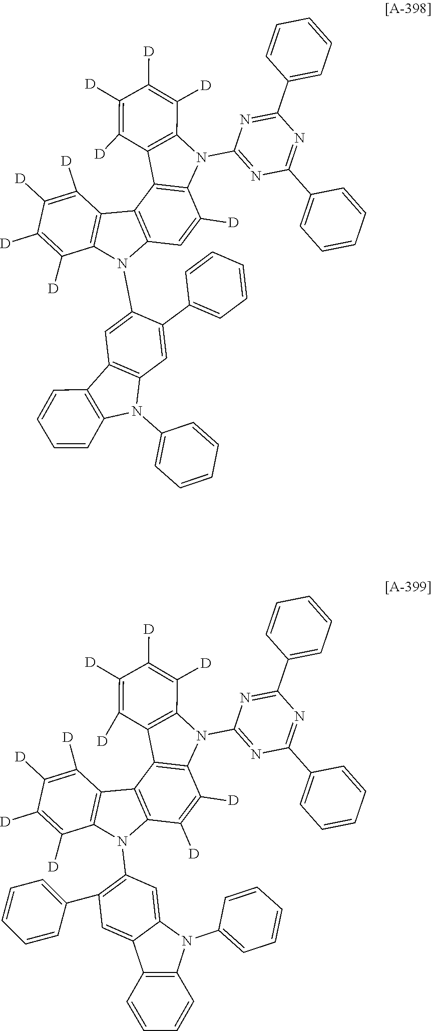 [SMILES: [2H]c1c([2H])c([2H])c2c(c1[2H])c1c3c4c([2H])c([2H])c([2H])c([2H])c4n(-c4nc(-c5ccccc5)nc(-c5ccccc5)n4)c3c([2H])cc1n2-c1cc2c3ccccc3n(-c3ccccc3)c2cc1-c1ccccc1 ; [2H]c1c([2H])c([2H])c2c(c1[2H])c1c3c4c([2H])c([2H])c([2H])c([2H])c4n(-c4cc5c(cc4-c4ccccc4)c4ccccc4n5-c4ccccc4)c3c([2H])c([2H])c1n2-c1nc(-c2ccccc2)nc(-c2ccccc2)n1]

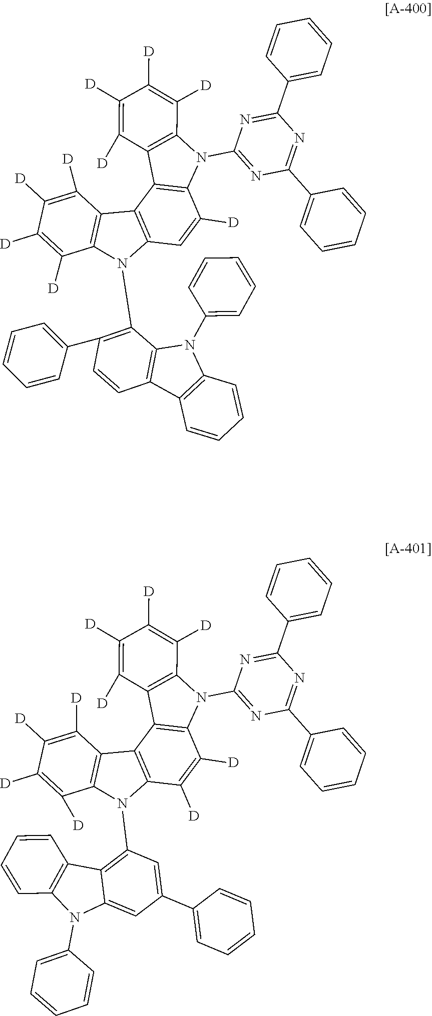 [SMILES: [2H]c1c([2H])c([2H])c2c(c1[2H])c1c3c4c([2H])c([2H])c([2H])c([2H])c4n(-c4nc(-c5ccccc5)nc(-c5ccccc5)n4)c3c([2H])cc1n2-c1c(-c2ccccc2)ccc2c3ccccc3n(-c3ccccc3)c12 ; [2H]c1c([2H])c([2H])c2c(c1[2H])c1c3c4c([2H])c([2H])c([2H])c([2H])c4n(-c4cc(-c5ccccc5)cc5c4c4ccccc4n5-c4ccccc4)c3c([2H])c([2H])c1n2-c1nc(-c2ccccc2)nc(-c2ccccc2)n1]

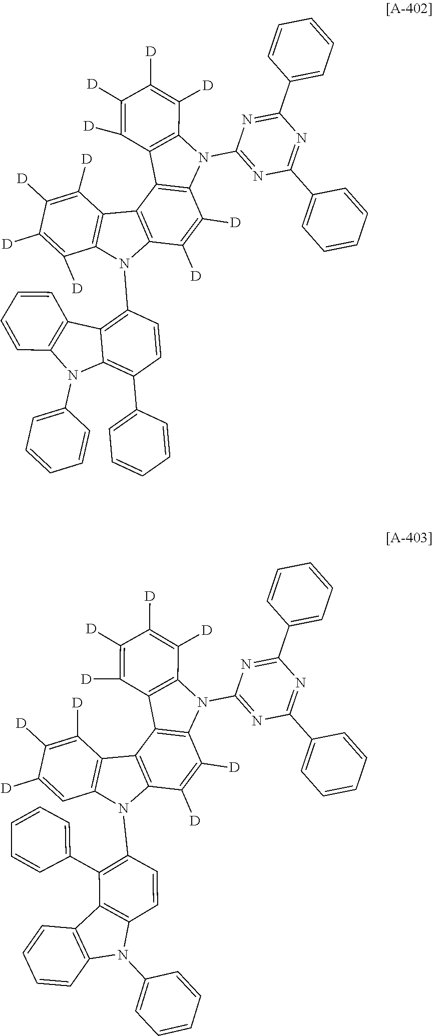 [SMILES: [2H]c1c([2H])c([2H])c2c(c1[2H])c1c3c4c([2H])c([2H])c([2H])c([2H])c4n(-c4ccc(-c5ccccc5)c5c4c4ccccc4n5-c4ccccc4)c3c([2H])c([2H])c1n2-c1nc(-c2ccccc2)nc(-c2ccccc2)n1 ; [2H]c1cc2c(c([2H])c1[2H])c1c3c4c([2H])c([2H])c([2H])c([2H])c4n(-c4nc(-c5ccccc5)nc(-c5ccccc5)n4)c3c([2H])c([2H])c1n2-c1ccc2c(c1-c1ccccc1)c1ccccc1n2-c1ccccc1]

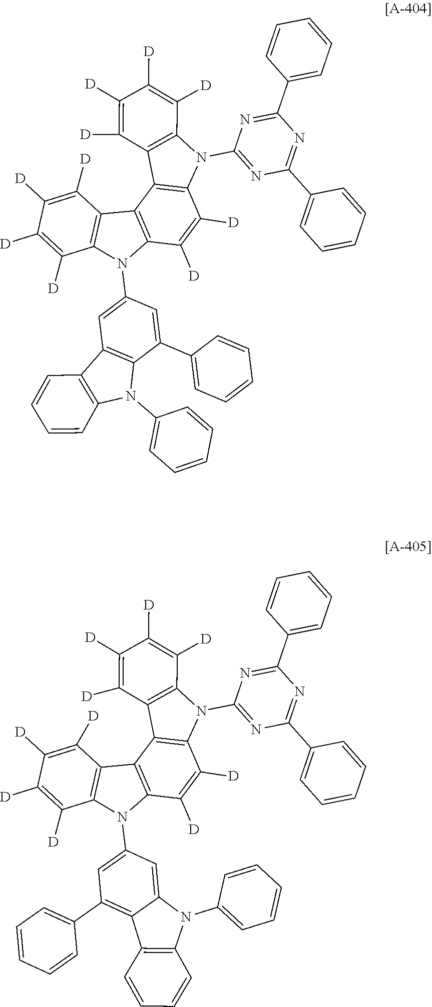 [SMILES: [2H]c1c([2H])c([2H])c2c(c1[2H])c1c3c4c([2H])c([2H])c([2H])c([2H])c4n(-c4nc(-c5ccccc5)nc(-c5ccccc5)n4)c3c([2H])c([2H])c1n2-c1cc(-c2ccccc2)c2c(c1)c1ccccc1n2-c1ccccc1 ; [2H]c1c([2H])c([2H])c2c(c1[2H])c1c3c4c([2H])c([2H])c([2H])c([2H])c4n(-c4nc(-c5ccccc5)nc(-c5ccccc5)n4)c3c([2H])c([2H])c1n2-c1cc(-c2ccccc2)c2c3ccccc3n(-c3ccccc3)c2c1]

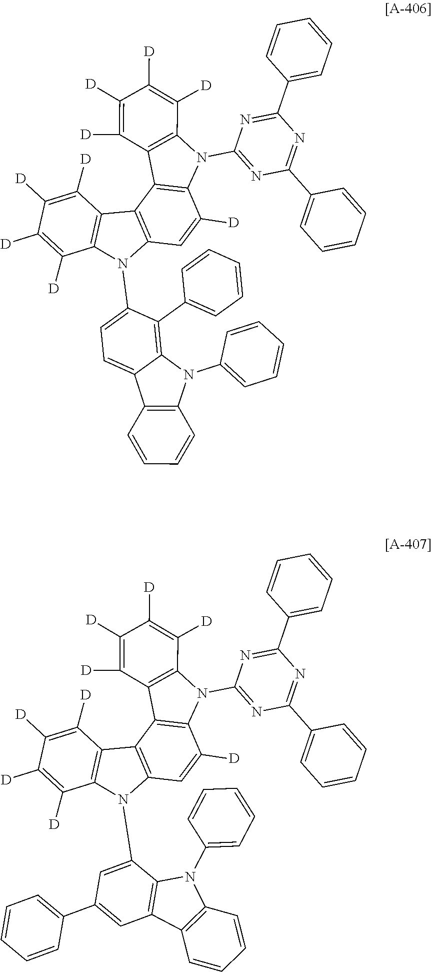 [SMILES: [2H]c1c([2H])c([2H])c2c(c1[2H])c1c3c4c([2H])c([2H])c([2H])c([2H])c4n(-c4nc(-c5ccccc5)nc(-c5ccccc5)n4)c3c([2H])cc1n2-c1ccc2c3ccccc3n(-c3ccccc3)c2c1-c1ccccc1 ; [2H]c1c([2H])c([2H])c2c(c1[2H])c1c3c4c([2H])c([2H])c([2H])c([2H])c4n(-c4nc(-c5ccccc5)nc(-c5ccccc5)n4)c3c([2H])cc1n2-c1cc(-c2ccccc2)cc2c3ccccc3n(-c3ccccc3)c12]

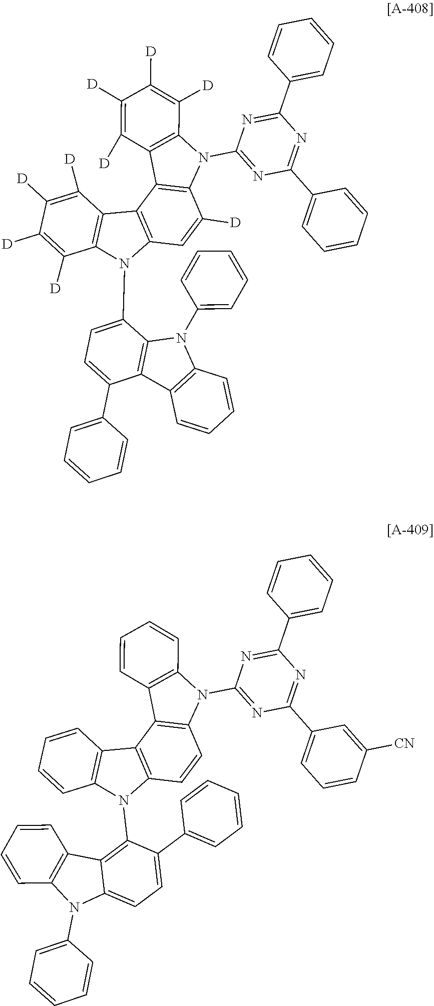 [SMILES: [2H]c1c([2H])c([2H])c2c(c1[2H])c1c3c4c([2H])c([2H])c([2H])c([2H])c4n(-c4nc(-c5ccccc5)nc(-c5ccccc5)n4)c3c([2H])cc1n2-c1ccc(-c2ccccc2)c2c3ccccc3n(-c3ccccc3)c12 ; N#Cc1cccc(-c2nc(-c3ccccc3)nc(-n3c4ccccc4c4c5c6ccccc6n(-c6c(-c7ccccc7)ccc7c6c6ccccc6n7-c6ccccc6)c5ccc43)n2)c1]

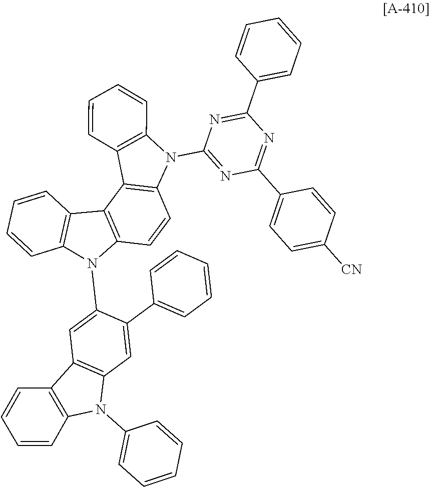 [SMILES: N#Cc1ccc(-c2nc(-c3ccccc3)nc(-n3c4ccccc4c4c5c6ccccc6n(-c6cc7c8ccccc8n(-c8ccccc8)c7cc6-c6ccccc6)c5ccc43)n2)cc1]

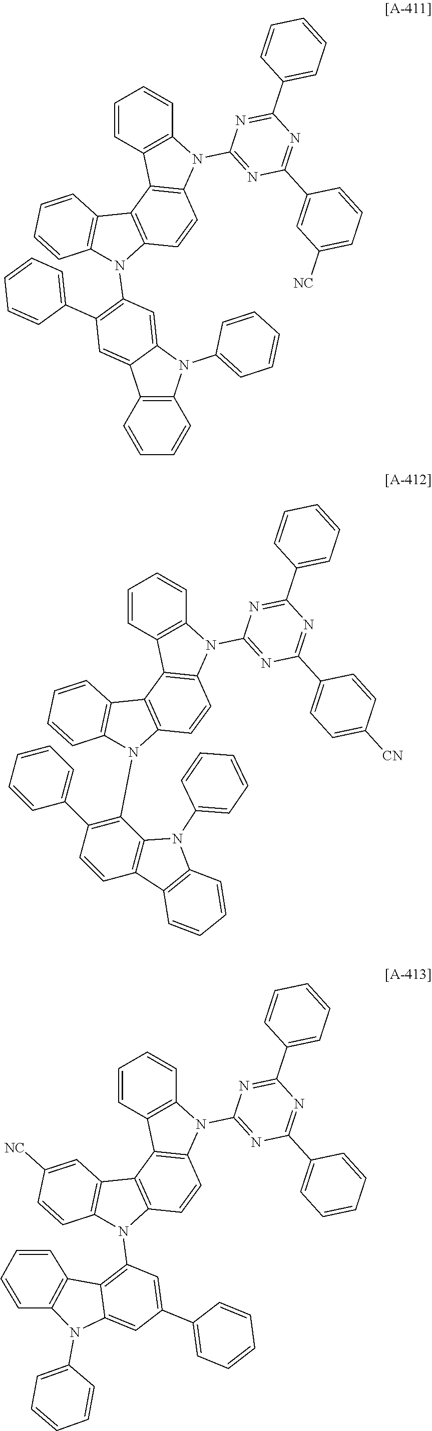 [SMILES: N#Cc1cccc(-c2nc(-c3ccccc3)nc(-n3c4ccccc4c4c5c6ccccc6n(-c6cc7c(cc6-c6ccccc6)c6ccccc6n7-c6ccccc6)c5ccc43)n2)c1 ; N#Cc1ccc(-c2nc(-c3ccccc3)nc(-n3c4ccccc4c4c5c6ccccc6n(-c6c(-c7ccccc7)ccc7c8ccccc8n(-c8ccccc8)c67)c5ccc43)n2)cc1 ; N#Cc1ccc2c(c1)c1c3c4ccccc4n(-c4nc(-c5ccccc5)nc(-c5ccccc5)n4)c3ccc1n2-c1cc(-c2ccccc2)cc2c1c1ccccc1n2-c1ccccc1]

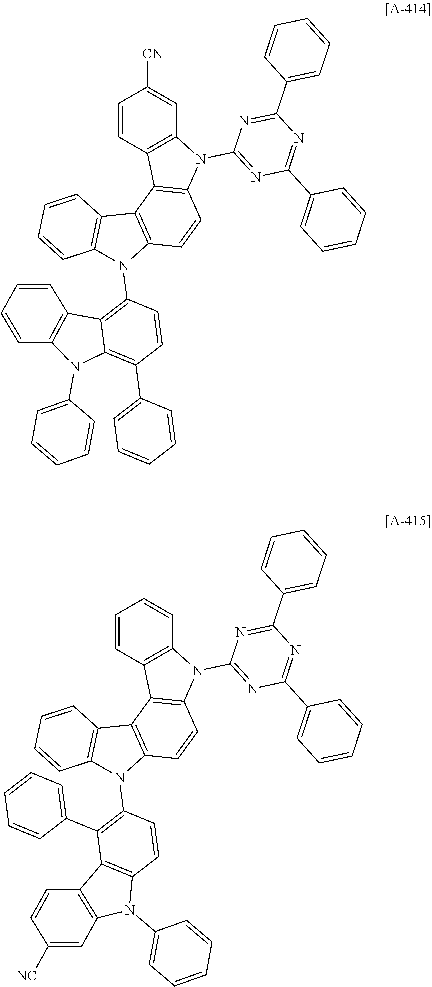 [SMILES: N#Cc1ccc2c3c4c5ccccc5n(-c5ccc(-c6ccccc6)c6c5c5ccccc5n6-c5ccccc5)c4ccc3n(-c3nc(-c4ccccc4)nc(-c4ccccc4)n3)c2c1 ; N#Cc1ccc2c3c(-c4ccccc4)c(-n4c5ccccc5c5c6c7ccccc7n(-c7nc(-c8ccccc8)nc(-c8ccccc8)n7)c6ccc54)ccc3n(-c3ccccc3)c2c1]

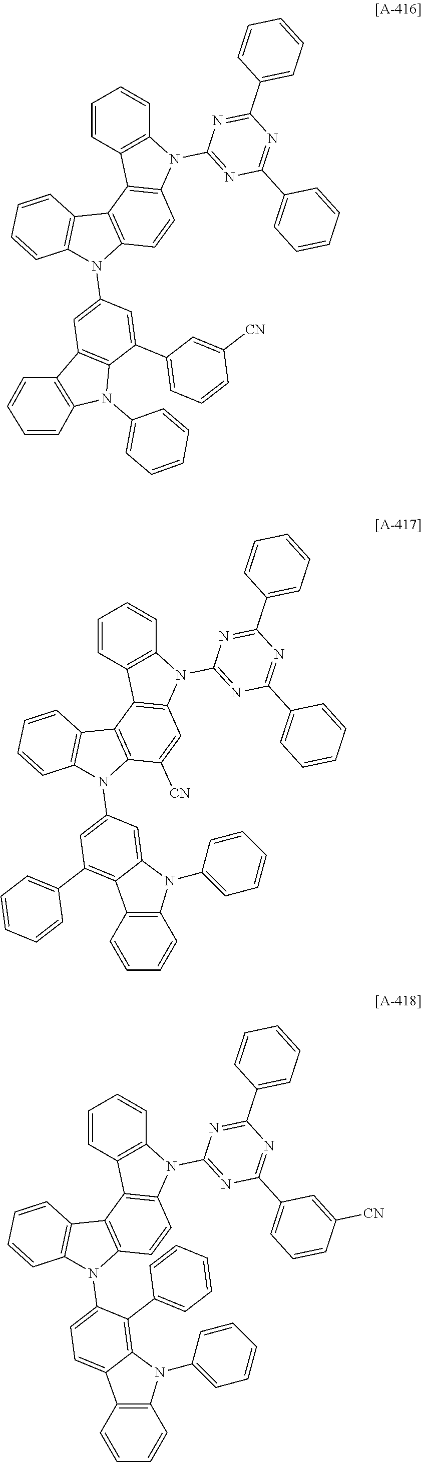 [SMILES: N#Cc1cccc(-c2cc(-n3c4ccccc4c4c5c6ccccc6n(-c6nc(-c7ccccc7)nc(-c7ccccc7)n6)c5ccc43)cc3c4ccccc4n(-c4ccccc4)c23)c1 ; N#Cc1cc2c(c3ccccc3n2-c2nc(-c3ccccc3)nc(-c3ccccc3)n2)c2c3ccccc3n(-c3cc(-c4ccccc4)c4c5ccccc5n(-c5ccccc5)c4c3)c12 ; N#Cc1cccc(-c2nc(-c3ccccc3)nc(-n3c4ccccc4c4c5c6ccccc6n(-c6ccc7c8ccccc8n(-c8ccccc8)c7c6-c6ccccc6)c5ccc43)n2)c1]

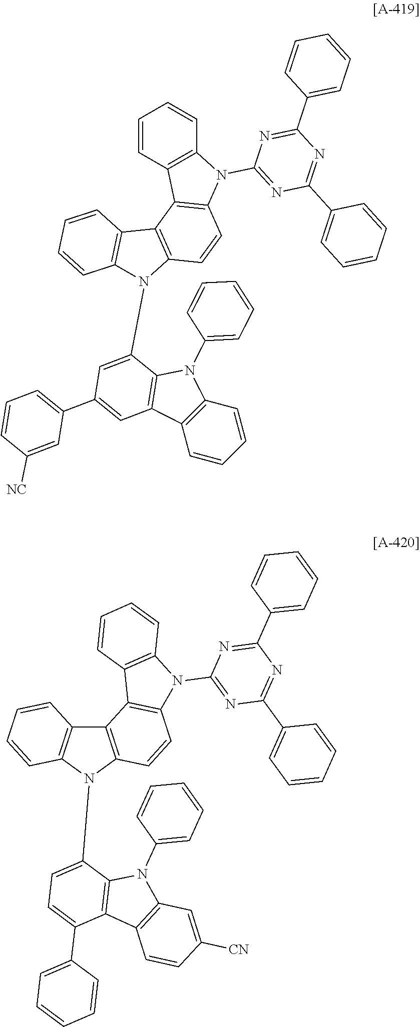 [SMILES: N#Cc1cccc(-c2cc(-n3c4ccccc4c4c5c6ccccc6n(-c6nc(-c7ccccc7)nc(-c7ccccc7)n6)c5ccc43)c3c(c2)c2ccccc2n3-c2ccccc2)c1 ; N#Cc1ccc2c3c(-c4ccccc4)ccc(-n4c5ccccc5c5c6c7ccccc7n(-c7nc(-c8ccccc8)nc(-c8ccccc8)n7)c6ccc54)c3n(-c3ccccc3)c2c1]

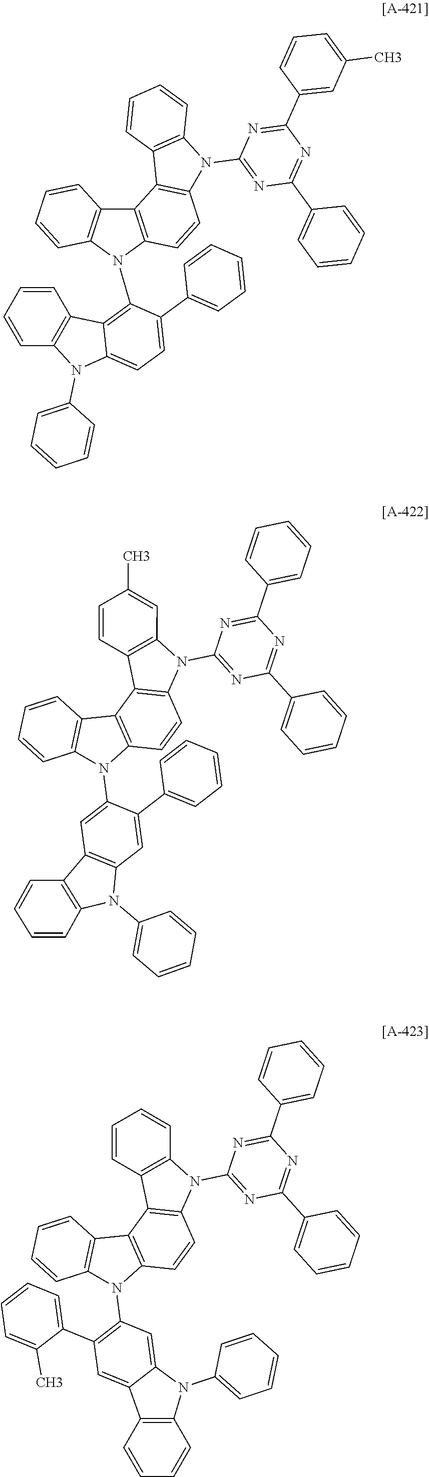 [SMILES: Cc1cccc(-c2nc(-c3ccccc3)nc(-n3c4ccccc4c4c5c6ccccc6n(-c6c(-c7ccccc7)ccc7c6c6ccccc6n7-c6ccccc6)c5ccc43)n2)c1 ; Cc1ccc2c3c4c5ccccc5n(-c5cc6c7ccccc7n(-c7ccccc7)c6cc5-c5ccccc5)c4ccc3n(-c3nc(-c4ccccc4)nc(-c4ccccc4)n3)c2c1 ; Cc1ccccc1-c1cc2c3ccccc3n(-c3ccccc3)c2cc1-n1c2ccccc2c2c3c4ccccc4n(-c4nc(-c5ccccc5)nc(-c5ccccc5)n4)c3ccc21]

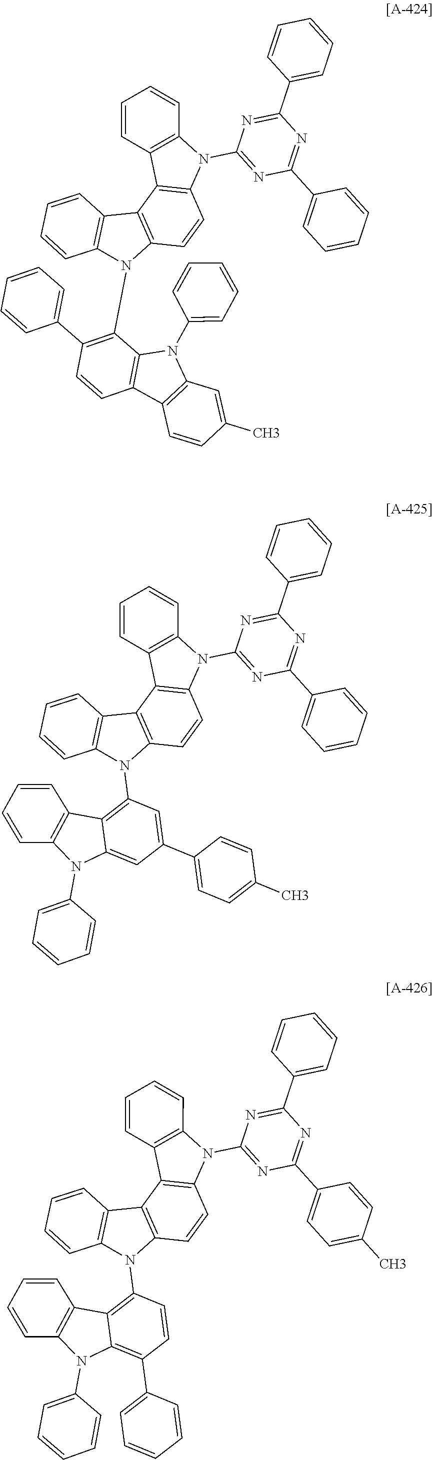 [SMILES: Cc1ccc2c3ccc(-c4ccccc4)c(-n4c5ccccc5c5c6c7ccccc7n(-c7nc(-c8ccccc8)nc(-c8ccccc8)n7)c6ccc54)c3n(-c3ccccc3)c2c1 ; Cc1ccc(-c2cc(-n3c4ccccc4c4c5c6ccccc6n(-c6nc(-c7ccccc7)nc(-c7ccccc7)n6)c5ccc43)c3c4ccccc4n(-c4ccccc4)c3c2)cc1 ; Cc1ccc(-c2nc(-c3ccccc3)nc(-n3c4ccccc4c4c5c6ccccc6n(-c6ccc(-c7ccccc7)c7c6c6ccccc6n7-c6ccccc6)c5ccc43)n2)cc1]

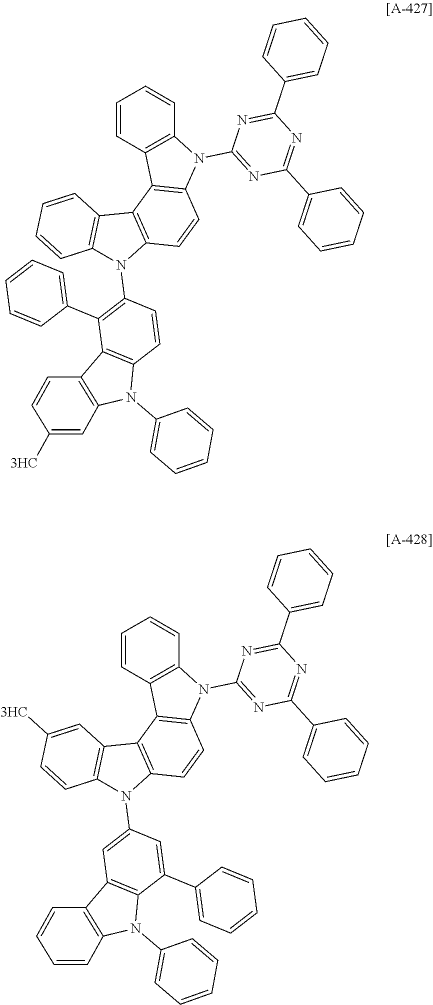

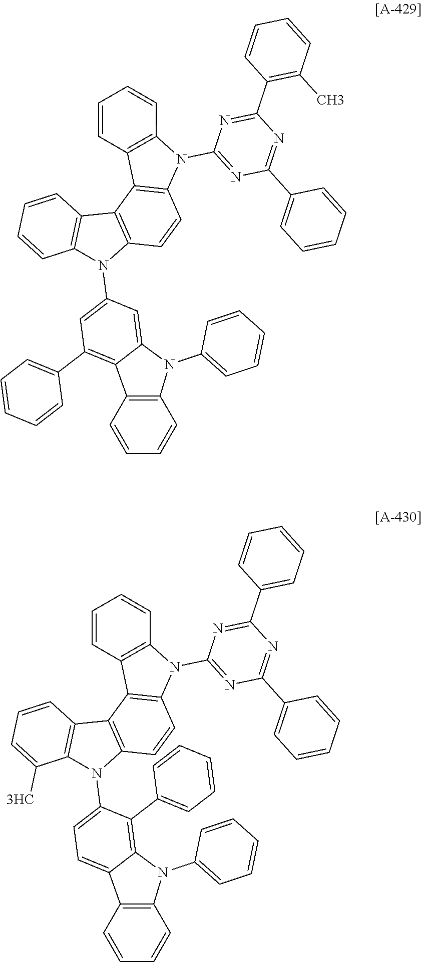

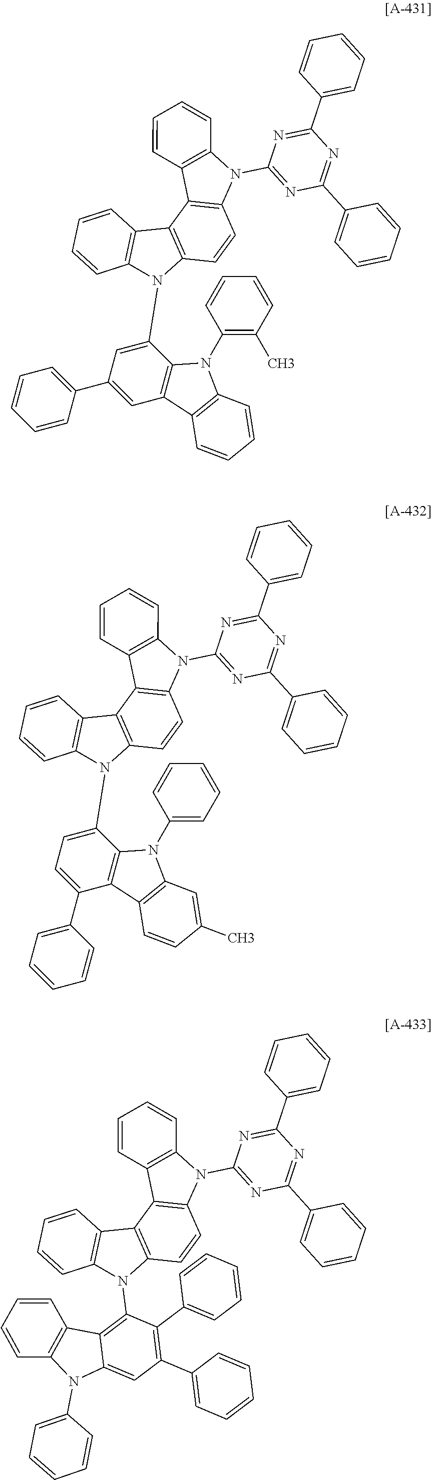 [SMILES: Cc1ccccc1-n1c2ccccc2c2cc(-c3ccccc3)cc(-n3c4ccccc4c4c5c6ccccc6n(-c6nc(-c7ccccc7)nc(-c7ccccc7)n6)c5ccc43)c21 ; Cc1ccc2c3c(-c4ccccc4)ccc(-n4c5ccccc5c5c6c7ccccc7n(-c7nc(-c8ccccc8)nc(-c8ccccc8)n7)c6ccc54)c3n(-c3ccccc3)c2c1 ; c1ccc(-c2nc(-c3ccccc3)nc(-n3c4ccccc4c4c5c6ccccc6n(-c6c(-c7ccccc7)c(-c7ccccc7)cc7c6c6ccccc6n7-c6ccccc6)c5ccc43)n2)cc1]

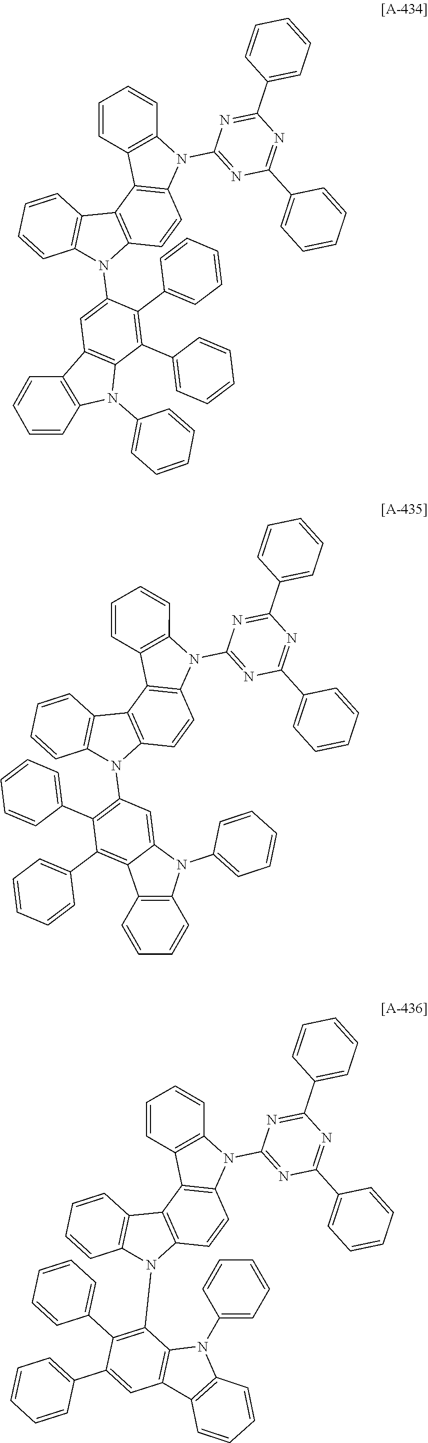 [SMILES: c1ccc(-c2nc(-c3ccccc3)nc(-n3c4ccccc4c4c5c6ccccc6n(-c6cc7c8ccccc8n(-c8ccccc8)c7c(-c7ccccc7)c6-c6ccccc6)c5ccc43)n2)cc1 ; c1ccc(-c2nc(-c3ccccc3)nc(-n3c4ccccc4c4c5c6ccccc6n(-c6cc7c(c(-c8ccccc8)c6-c6ccccc6)c6ccccc6n7-c6ccccc6)c5ccc43)n2)cc1 ; c1ccc(-c2nc(-c3ccccc3)nc(-n3c4ccccc4c4c5c6ccccc6n(-c6c(-c7ccccc7)c(-c7ccccc7)cc7c8ccccc8n(-c8ccccc8)c67)c5ccc43)n2)cc1]

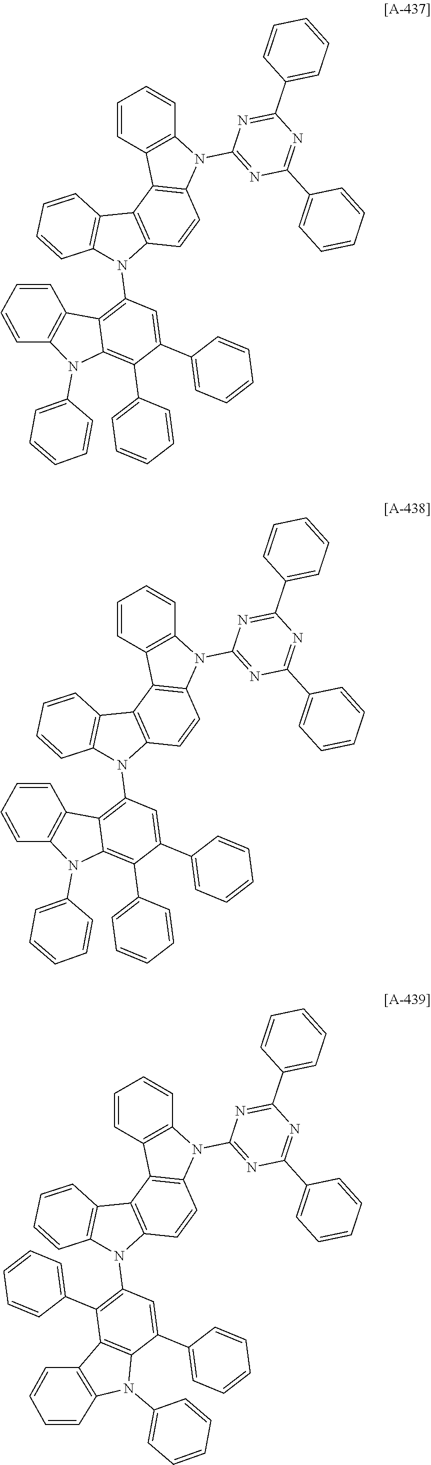 [SMILES: c1ccc(-c2nc(-c3ccccc3)nc(-n3c4ccccc4c4c5c6ccccc6n(-c6cc(-c7ccccc7)c(-c7ccccc7)c7c6c6ccccc6n7-c6ccccc6)c5ccc43)n2)cc1 ; c1ccc(-c2nc(-c3ccccc3)nc(-n3c4ccccc4c4c5c6ccccc6n(-c6cc(-c7ccccc7)c(-c7ccccc7)c7c6c6ccccc6n7-c6ccccc6)c5ccc43)n2)cc1 ; c1ccc(-c2nc(-c3ccccc3)nc(-n3c4ccccc4c4c5c6ccccc6n(-c6cc(-c7ccccc7)c7c(c6-c6ccccc6)c6ccccc6n7-c6ccccc6)c5ccc43)n2)cc1]

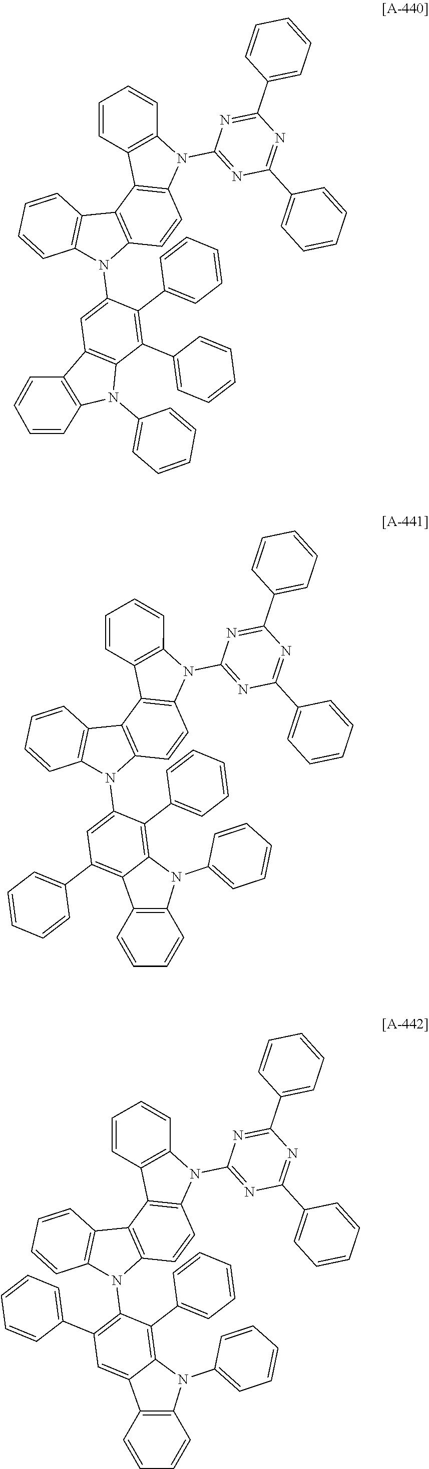 [SMILES: c1ccc(-c2nc(-c3ccccc3)nc(-n3c4ccccc4c4c5c6ccccc6n(-c6cc7c8ccccc8n(-c8ccccc8)c7c(-c7ccccc7)c6-c6ccccc6)c5ccc43)n2)cc1 ; c1ccc(-c2nc(-c3ccccc3)nc(-n3c4ccccc4c4c5c6ccccc6n(-c6cc(-c7ccccc7)c7c8ccccc8n(-c8ccccc8)c7c6-c6ccccc6)c5ccc43)n2)cc1 ; c1ccc(-c2nc(-c3ccccc3)nc(-n3c4ccccc4c4c5c6ccccc6n(-c6c(-c7ccccc7)cc7c8ccccc8n(-c8ccccc8)c7c6-c6ccccc6)c5ccc43)n2)cc1]

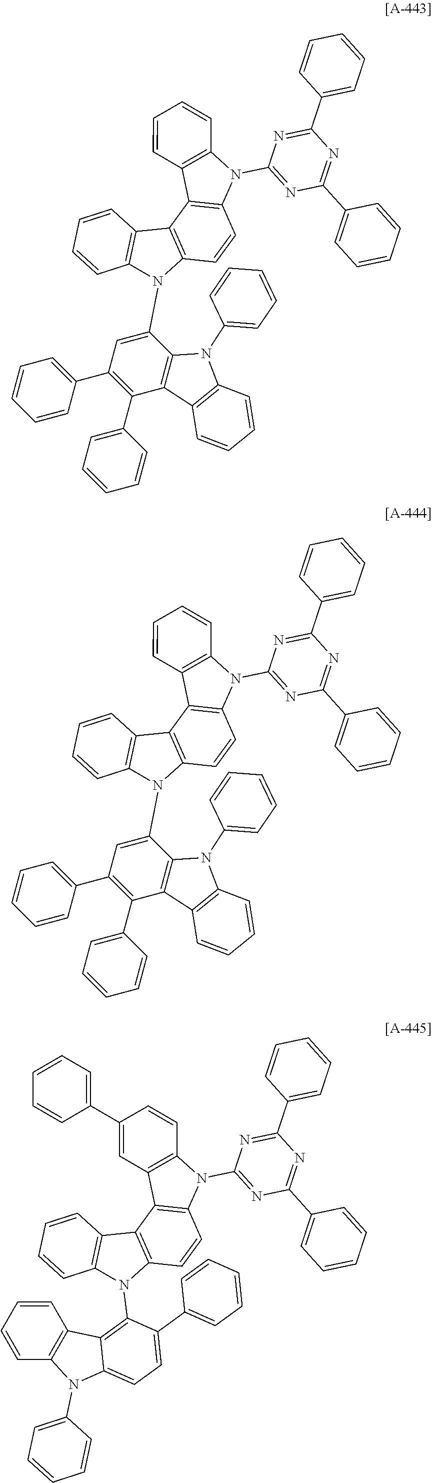 [SMILES: c1ccc(-c2nc(-c3ccccc3)nc(-n3c4ccccc4c4c5c6ccccc6n(-c6cc(-c7ccccc7)c(-c7ccccc7)c7c8ccccc8n(-c8ccccc8)c67)c5ccc43)n2)cc1 ; c1ccc(-c2nc(-c3ccccc3)nc(-n3c4ccccc4c4c5c6ccccc6n(-c6cc(-c7ccccc7)c(-c7ccccc7)c7c8ccccc8n(-c8ccccc8)c67)c5ccc43)n2)cc1 ; c1ccc(-c2ccc3c(c2)c2c4c5ccccc5n(-c5c(-c6ccccc6)ccc6c5c5ccccc5n6-c5ccccc5)c4ccc2n3-c2nc(-c3ccccc3)nc(-c3ccccc3)n2)cc1]

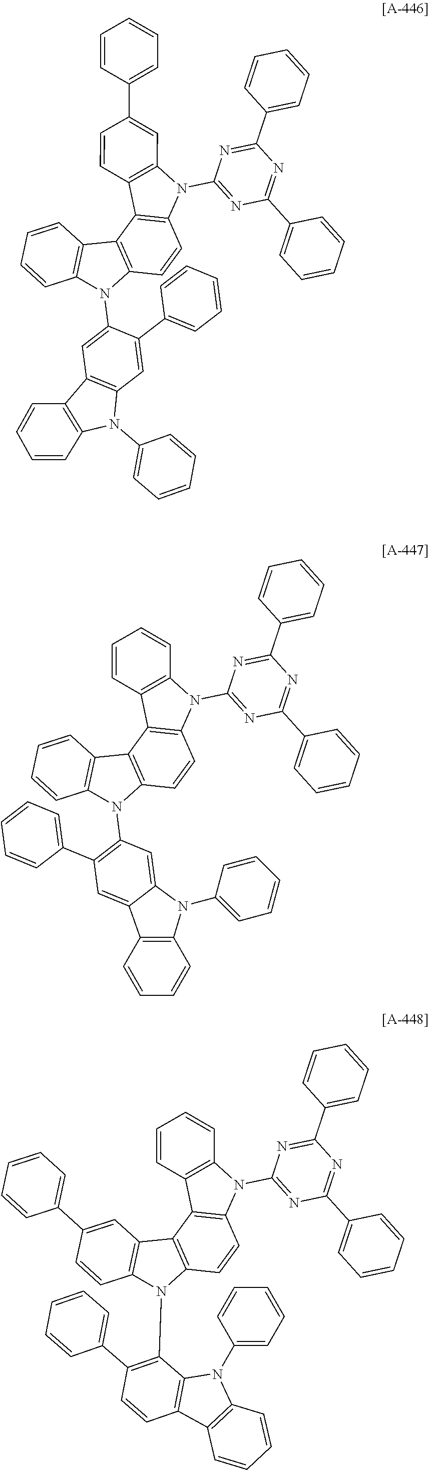 [SMILES: c1ccc(-c2ccc3c4c5c6ccccc6n(-c6cc7c8ccccc8n(-c8ccccc8)c7cc6-c6ccccc6)c5ccc4n(-c4nc(-c5ccccc5)nc(-c5ccccc5)n4)c3c2)cc1 ; c1ccc(-c2nc(-c3ccccc3)nc(-n3c4ccccc4c4c5c6ccccc6n(-c6cc7c(cc6-c6ccccc6)c6ccccc6n7-c6ccccc6)c5ccc43)n2)cc1 ; c1ccc(-c2ccc3c(c2)c2c4c5ccccc5n(-c5nc(-c6ccccc6)nc(-c6ccccc6)n5)c4ccc2n3-c2c(-c3ccccc3)ccc3c4ccccc4n(-c4ccccc4)c23)cc1]

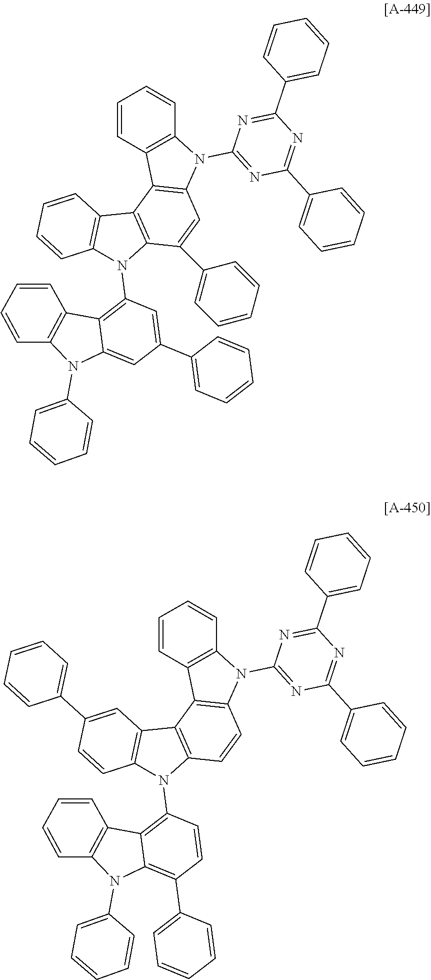 [SMILES: c1ccc(-c2cc(-n3c4ccccc4c4c5c6ccccc6n(-c6nc(-c7ccccc7)nc(-c7ccccc7)n6)c5cc(-c5ccccc5)c43)c3c4ccccc4n(-c4ccccc4)c3c2)cc1 ; c1ccc(-c2ccc3c(c2)c2c4c5ccccc5n(-c5nc(-c6ccccc6)nc(-c6ccccc6)n5)c4ccc2n3-c2ccc(-c3ccccc3)c3c2c2ccccc2n3-c2ccccc2)cc1]

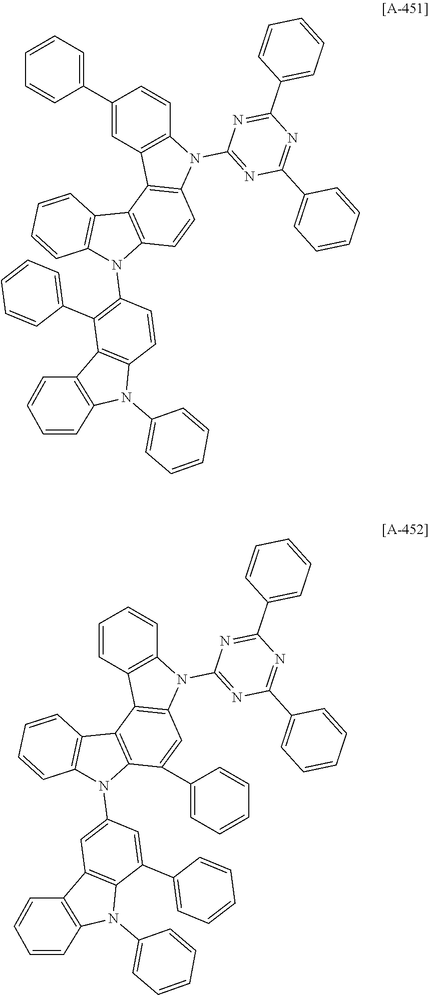 [SMILES: c1ccc(-c2ccc3c(c2)c2c4c5ccccc5n(-c5ccc6c(c5-c5ccccc5)c5ccccc5n6-c5ccccc5)c4ccc2n3-c2nc(-c3ccccc3)nc(-c3ccccc3)n2)cc1 ; c1ccc(-c2nc(-c3ccccc3)nc(-n3c4ccccc4c4c5c6ccccc6n(-c6cc(-c7ccccc7)c7c(c6)c6ccccc6n7-c6ccccc6)c5c(-c5ccccc5)cc43)n2)cc1]

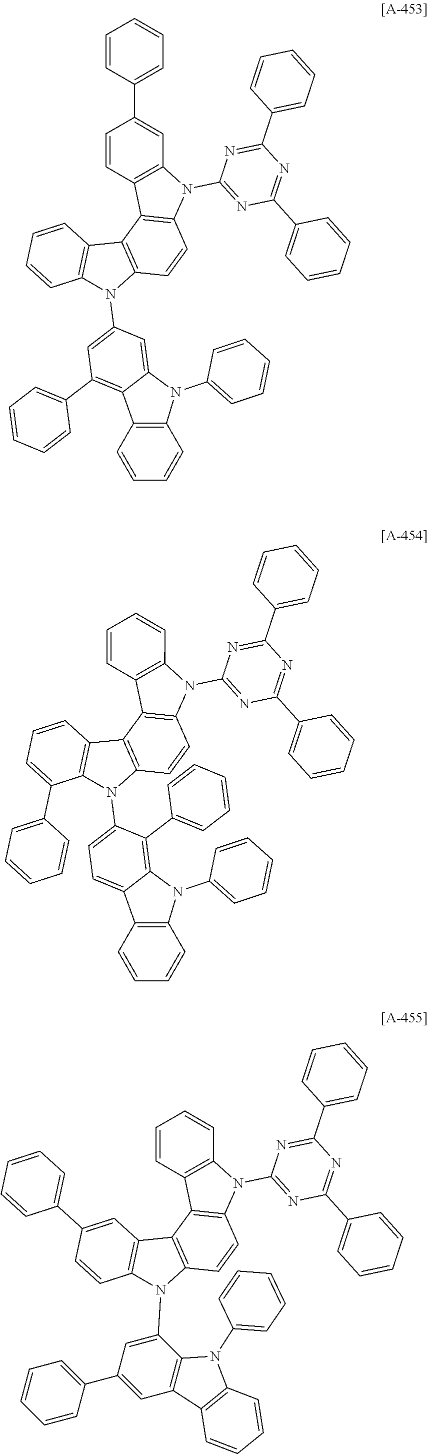 [SMILES: c1ccc(-c2ccc3c4c5c6ccccc6n(-c6cc(-c7ccccc7)c7c8ccccc8n(-c8ccccc8)c7c6)c5ccc4n(-c4nc(-c5ccccc5)nc(-c5ccccc5)n4)c3c2)cc1 ; c1ccc(-c2nc(-c3ccccc3)nc(-n3c4ccccc4c4c5c6cccc(-c7ccccc7)c6n(-c6ccc7c8ccccc8n(-c8ccccc8)c7c6-c6ccccc6)c5ccc43)n2)cc1 ; c1ccc(-c2ccc3c(c2)c2c4c5ccccc5n(-c5nc(-c6ccccc6)nc(-c6ccccc6)n5)c4ccc2n3-c2cc(-c3ccccc3)cc3c4ccccc4n(-c4ccccc4)c23)cc1]

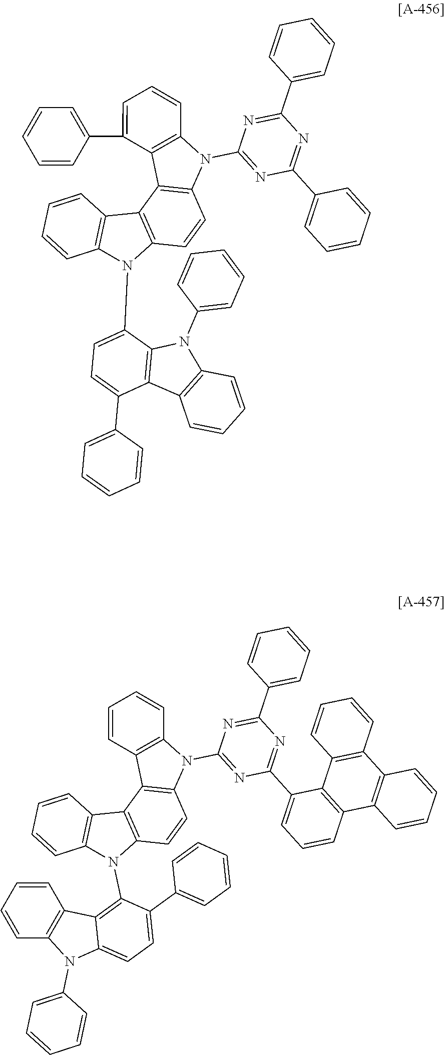 [SMILES: c1ccc(-c2nc(-c3ccccc3)nc(-n3c4cccc(-c5ccccc5)c4c4c5c6ccccc6n(-c6ccc(-c7ccccc7)c7c8ccccc8n(-c8ccccc8)c67)c5ccc43)n2)cc1 ; c1ccc(-c2nc(-c3cccc4c5ccccc5c5ccccc5c34)nc(-n3c4ccccc4c4c5c6ccccc6n(-c6c(-c7ccccc7)ccc7c6c6ccccc6n7-c6ccccc6)c5ccc43)n2)cc1]

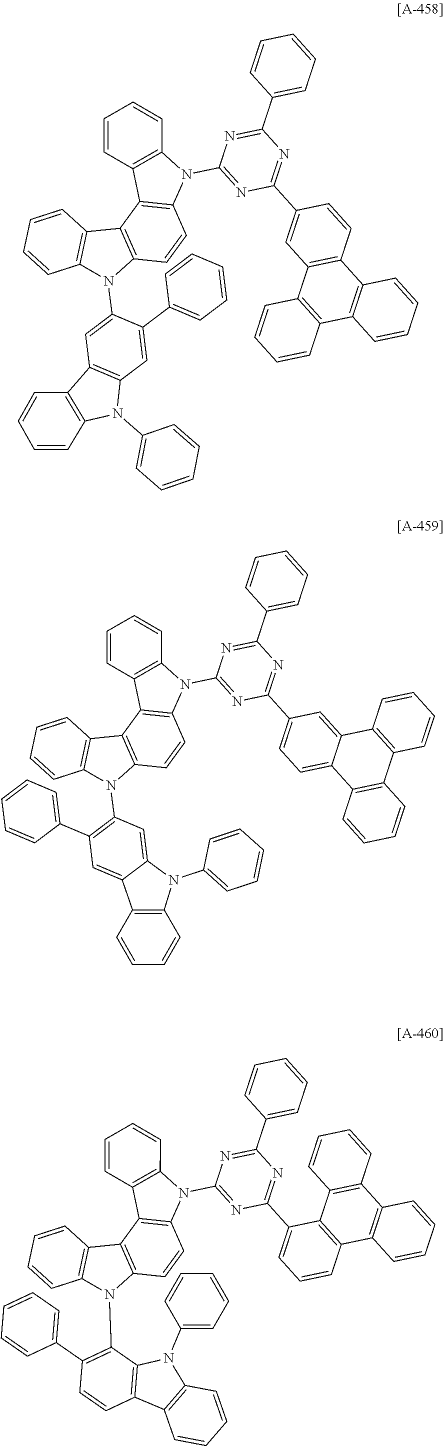 [SMILES: c1ccc(-c2nc(-c3ccc4c5ccccc5c5ccccc5c4c3)nc(-n3c4ccccc4c4c5c6ccccc6n(-c6cc7c8ccccc8n(-c8ccccc8)c7cc6-c6ccccc6)c5ccc43)n2)cc1 ; c1ccc(-c2nc(-c3ccc4c5ccccc5c5ccccc5c4c3)nc(-n3c4ccccc4c4c5c6ccccc6n(-c6cc7c(cc6-c6ccccc6)c6ccccc6n7-c6ccccc6)c5ccc43)n2)cc1 ; c1ccc(-c2nc(-c3cccc4c5ccccc5c5ccccc5c34)nc(-n3c4ccccc4c4c5c6ccccc6n(-c6c(-c7ccccc7)ccc7c8ccccc8n(-c8ccccc8)c67)c5ccc43)n2)cc1]

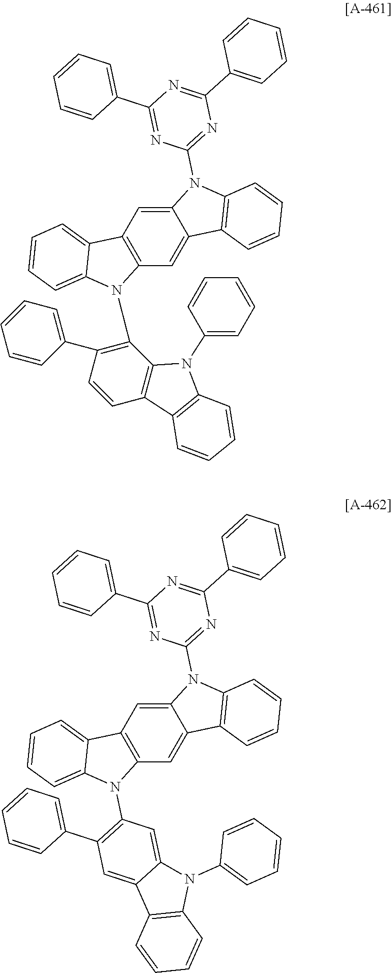 [SMILES: c1ccc(-c2nc(-c3ccccc3)nc(-n3c4ccccc4c4cc5c(cc43)c3ccccc3n5-c3c(-c4ccccc4)ccc4c5ccccc5n(-c5ccccc5)c34)n2)cc1 ; c1ccc(-c2nc(-c3ccccc3)nc(-n3c4ccccc4c4cc5c(cc43)c3ccccc3n5-c3cc4c(cc3-c3ccccc3)c3ccccc3n4-c3ccccc3)n2)cc1]

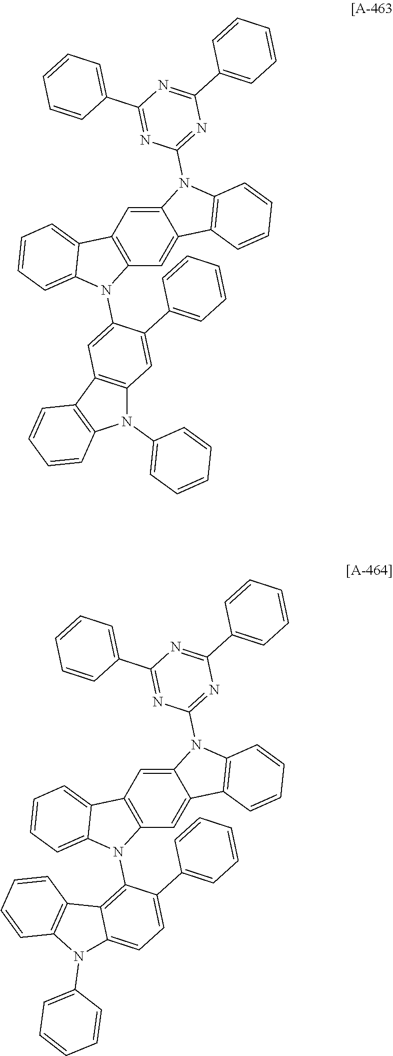 [SMILES: c1ccc(-c2nc(-c3ccccc3)nc(-n3c4ccccc4c4cc5c(cc43)c3ccccc3n5-c3cc4c5ccccc5n(-c5ccccc5)c4cc3-c3ccccc3)n2)cc1 ; c1ccc(-c2nc(-c3ccccc3)nc(-n3c4ccccc4c4cc5c(cc43)c3ccccc3n5-c3c(-c4ccccc4)ccc4c3c3ccccc3n4-c3ccccc3)n2)cc1]

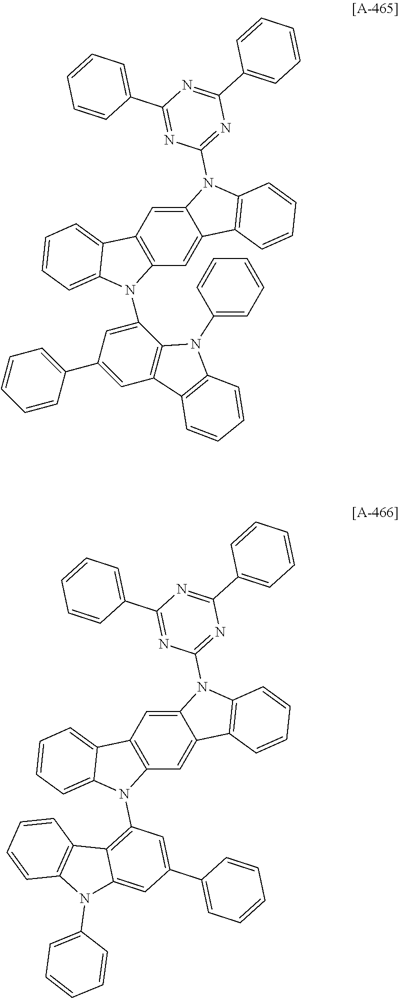 [SMILES: c1ccc(-c2cc(-n3c4ccccc4c4cc5c(cc43)c3ccccc3n5-c3nc(-c4ccccc4)nc(-c4ccccc4)n3)c3c(c2)c2ccccc2n3-c2ccccc2)cc1 ; c1ccc(-c2cc(-n3c4ccccc4c4cc5c(cc43)c3ccccc3n5-c3nc(-c4ccccc4)nc(-c4ccccc4)n3)c3c4ccccc4n(-c4ccccc4)c3c2)cc1]

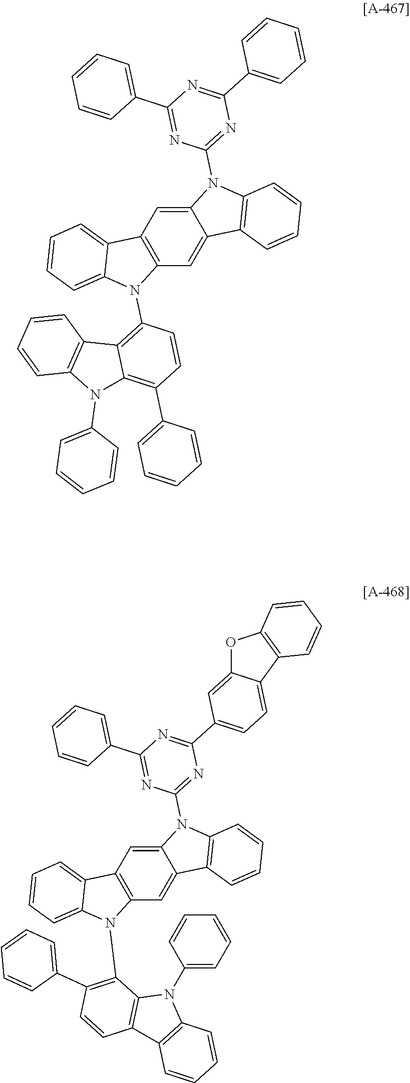 [SMILES: c1ccc(-c2nc(-c3ccccc3)nc(-n3c4ccccc4c4cc5c(cc43)c3ccccc3n5-c3ccc(-c4ccccc4)c4c3c3ccccc3n4-c3ccccc3)n2)cc1 ; c1ccc(-c2nc(-c3ccc4c(c3)oc3ccccc34)nc(-n3c4ccccc4c4cc5c(cc43)c3ccccc3n5-c3c(-c4ccccc4)ccc4c5ccccc5n(-c5ccccc5)c34)n2)cc1]

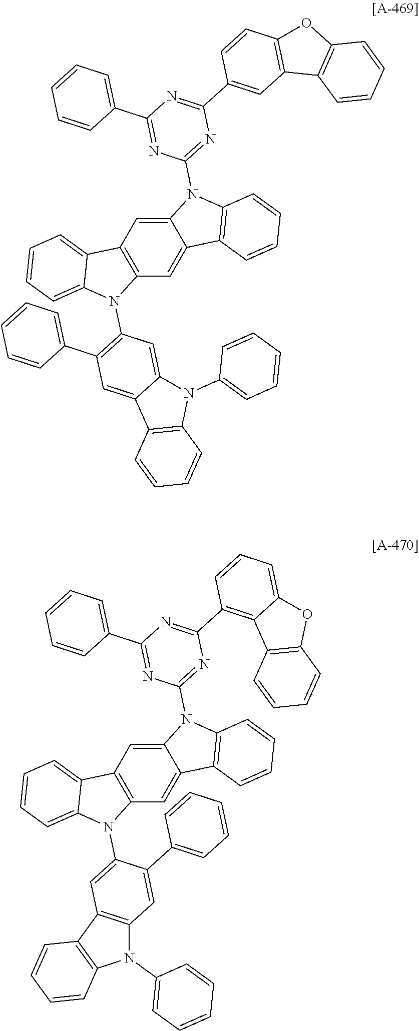 [SMILES: c1ccc(-c2nc(-c3ccc4oc5ccccc5c4c3)nc(-n3c4ccccc4c4cc5c(cc43)c3ccccc3n5-c3cc4c(cc3-c3ccccc3)c3ccccc3n4-c3ccccc3)n2)cc1 ; c1ccc(-c2nc(-c3cccc4oc5ccccc5c34)nc(-n3c4ccccc4c4cc5c(cc43)c3ccccc3n5-c3cc4c5ccccc5n(-c5ccccc5)c4cc3-c3ccccc3)n2)cc1]

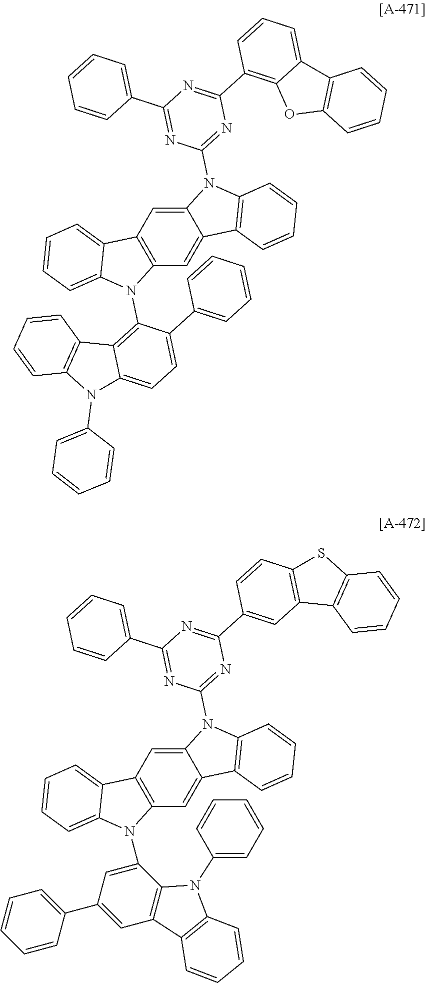 [SMILES: c1ccc(-c2nc(-c3cccc4c3oc3ccccc34)nc(-n3c4ccccc4c4cc5c(cc43)c3ccccc3n5-c3c(-c4ccccc4)ccc4c3c3ccccc3n4-c3ccccc3)n2)cc1 ; c1ccc(-c2cc(-n3c4ccccc4c4cc5c(cc43)c3ccccc3n5-c3nc(-c4ccccc4)nc(-c4ccc5sc6ccccc6c5c4)n3)c3c(c2)c2ccccc2n3-c2ccccc2)cc1]

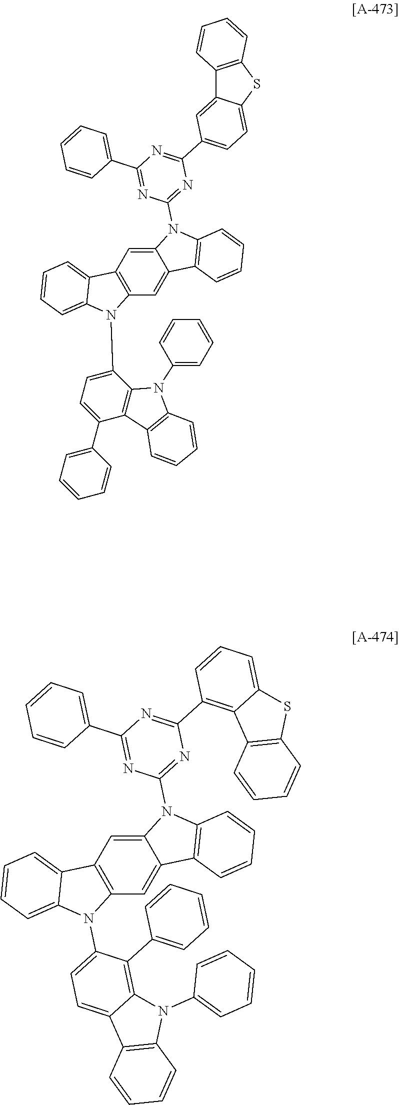 [SMILES: c1ccc(-c2nc(-c3ccc4sc5ccccc5c4c3)nc(-n3c4ccccc4c4cc5c(cc43)c3ccccc3n5-c3ccc(-c4ccccc4)c4c5ccccc5n(-c5ccccc5)c34)n2)cc1 ; c1ccc(-c2nc(-c3cccc4sc5ccccc5c34)nc(-n3c4ccccc4c4cc5c(cc43)c3ccccc3n5-c3ccc4c5ccccc5n(-c5ccccc5)c4c3-c3ccccc3)n2)cc1]

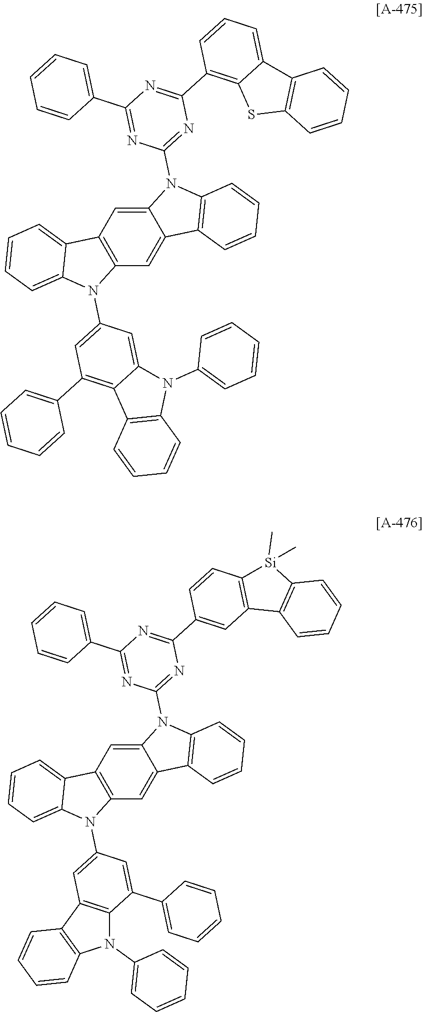 [SMILES: c1ccc(-c2nc(-c3cccc4c3sc3ccccc34)nc(-n3c4ccccc4c4cc5c(cc43)c3ccccc3n5-c3cc(-c4ccccc4)c4c5ccccc5n(-c5ccccc5)c4c3)n2)cc1 ; C[Si]1(C)c2ccccc2-c2cc(-c3nc(-c4ccccc4)nc(-n4c5ccccc5c5cc6c(cc54)c4ccccc4n6-c4cc(-c5ccccc5)c5c(c4)c4ccccc4n5-c4ccccc4)n3)ccc21]

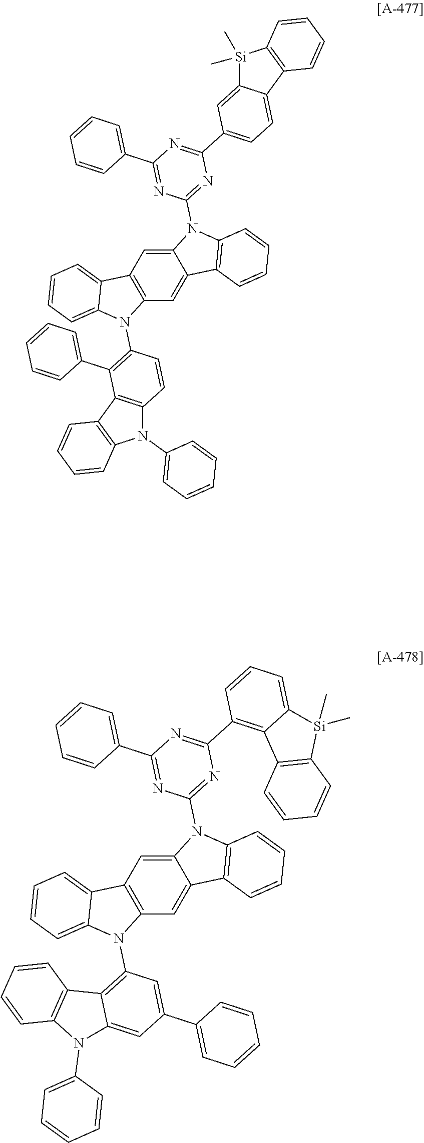 [SMILES: C[Si]1(C)c2ccccc2-c2ccc(-c3nc(-c4ccccc4)nc(-n4c5ccccc5c5cc6c(cc54)c4ccccc4n6-c4ccc5c(c4-c4ccccc4)c4ccccc4n5-c4ccccc4)n3)cc21 ; C[Si]1(C)c2ccccc2-c2c(-c3nc(-c4ccccc4)nc(-n4c5ccccc5c5cc6c(cc54)c4ccccc4n6-c4cc(-c5ccccc5)cc5c4c4ccccc4n5-c4ccccc4)n3)cccc21]

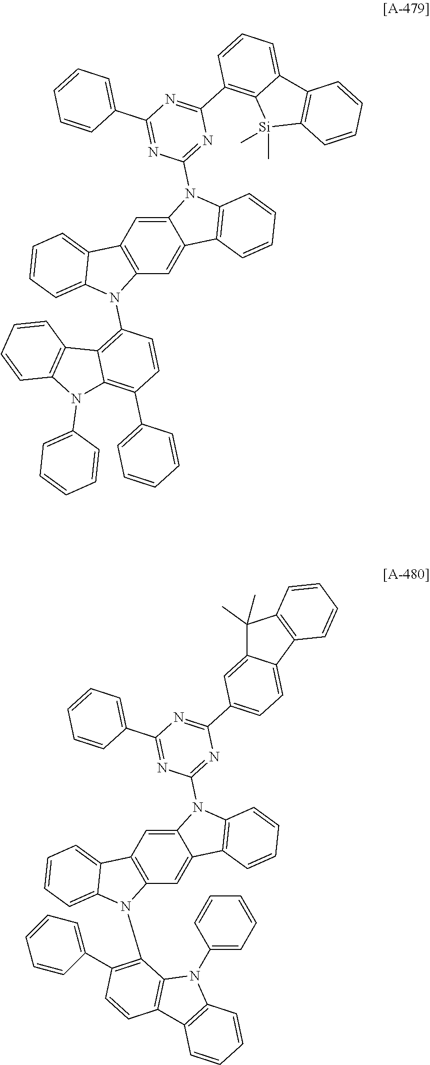 [SMILES: C[Si]1(C)c2ccccc2-c2cccc(-c3nc(-c4ccccc4)nc(-n4c5ccccc5c5cc6c(cc54)c4ccccc4n6-c4ccc(-c5ccccc5)c5c4c4ccccc4n5-c4ccccc4)n3)c21 ; CC1(C)c2ccccc2-c2ccc(-c3nc(-c4ccccc4)nc(-n4c5ccccc5c5cc6c(cc54)c4ccccc4n6-c4c(-c5ccccc5)ccc5c6ccccc6n(-c6ccccc6)c45)n3)cc21]

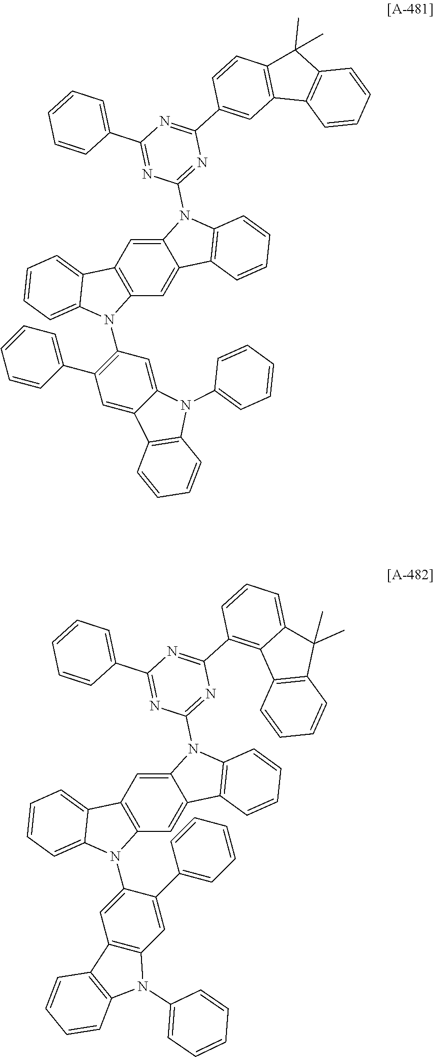 [SMILES: CC1(C)c2ccccc2-c2cc(-c3nc(-c4ccccc4)nc(-n4c5ccccc5c5cc6c(cc54)c4ccccc4n6-c4cc5c(cc4-c4ccccc4)c4ccccc4n5-c4ccccc4)n3)ccc21 ; CC1(C)c2ccccc2-c2c(-c3nc(-c4ccccc4)nc(-n4c5ccccc5c5cc6c(cc54)c4ccccc4n6-c4cc5c6ccccc6n(-c6ccccc6)c5cc4-c4ccccc4)n3)cccc21]

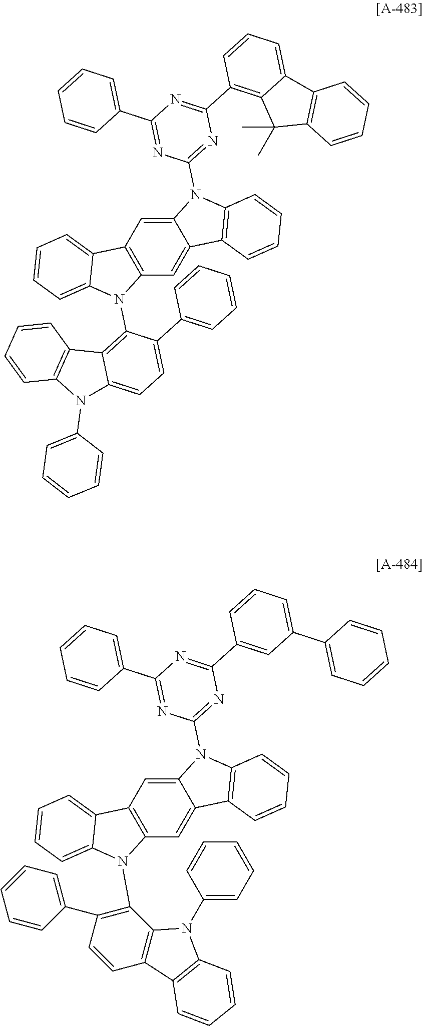 [SMILES: CC1(C)c2ccccc2-c2cccc(-c3nc(-c4ccccc4)nc(-n4c5ccccc5c5cc6c(cc54)c4ccccc4n6-c4c(-c5ccccc5)ccc5c4c4ccccc4n5-c4ccccc4)n3)c21 ; c1ccc(-c2cccc(-c3nc(-c4ccccc4)nc(-n4c5ccccc5c5cc6c(cc54)c4ccccc4n6-c4c(-c5ccccc5)ccc5c6ccccc6n(-c6ccccc6)c45)n3)c2)cc1]

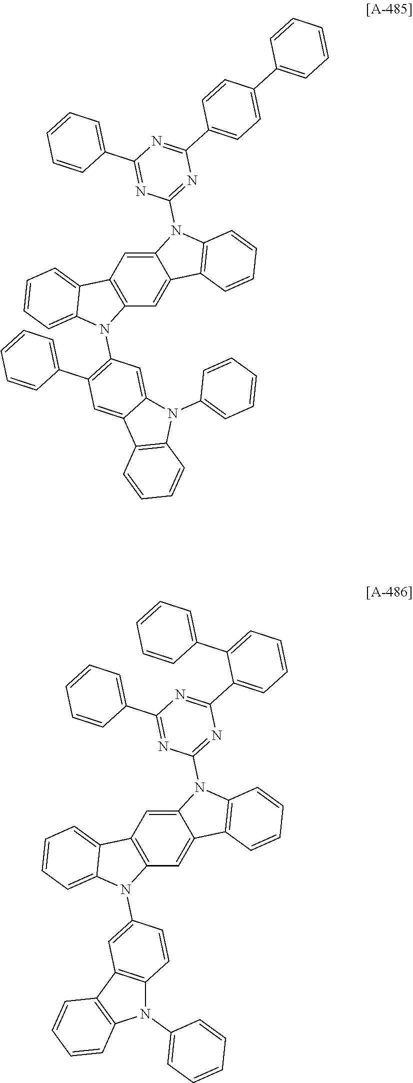 [SMILES: c1ccc(-c2ccc(-c3nc(-c4ccccc4)nc(-n4c5ccccc5c5cc6c(cc54)c4ccccc4n6-c4cc5c(cc4-c4ccccc4)c4ccccc4n5-c4ccccc4)n3)cc2)cc1 ; c1ccc(-c2nc(-c3ccccc3-c3ccccc3)nc(-n3c4ccccc4c4cc5c(cc43)c3ccccc3n5-c3ccc4c(c3)c3ccccc3n4-c3ccccc3)n2)cc1]

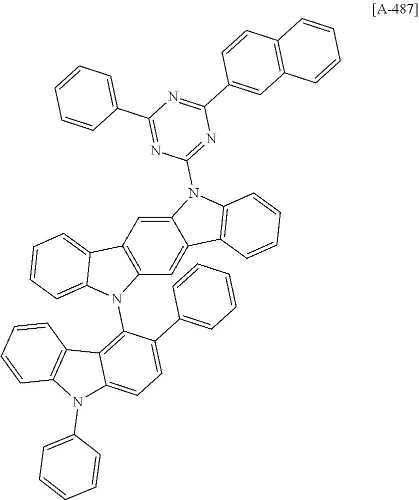 [SMILES: c1ccc(-c2nc(-c3ccc4ccccc4c3)nc(-n3c4ccccc4c4cc5c(cc43)c3ccccc3n5-c3c(-c4ccccc4)ccc4c3c3ccccc3n4-c3ccccc3)n2)cc1]

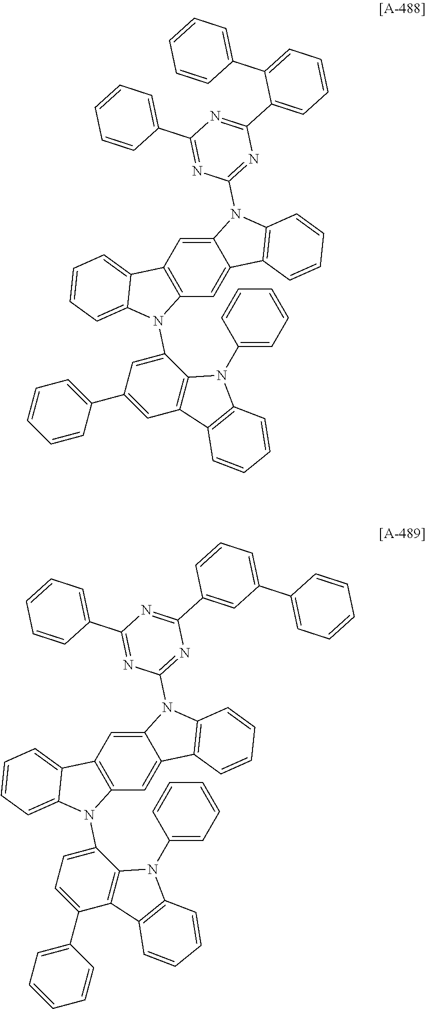 [SMILES: c1ccc(-c2cc(-n3c4ccccc4c4cc5c(cc43)c3ccccc3n5-c3nc(-c4ccccc4)nc(-c4ccccc4-c4ccccc4)n3)c3c(c2)c2ccccc2n3-c2ccccc2)cc1 ; c1ccc(-c2cccc(-c3nc(-c4ccccc4)nc(-n4c5ccccc5c5cc6c(cc54)c4ccccc4n6-c4ccc(-c5ccccc5)c5c6ccccc6n(-c6ccccc6)c45)n3)c2)cc1]

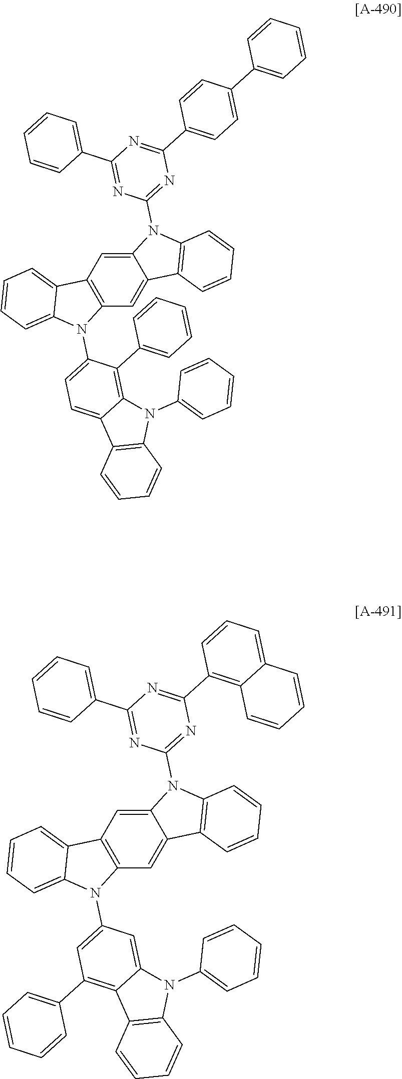 [SMILES: c1ccc(-c2ccc(-c3nc(-c4ccccc4)nc(-n4c5ccccc5c5cc6c(cc54)c4ccccc4n6-c4ccc5c6ccccc6n(-c6ccccc6)c5c4-c4ccccc4)n3)cc2)cc1 ; c1ccc(-c2nc(-c3cccc4ccccc34)nc(-n3c4ccccc4c4cc5c(cc43)c3ccccc3n5-c3cc(-c4ccccc4)c4c5ccccc5n(-c5ccccc5)c4c3)n2)cc1]

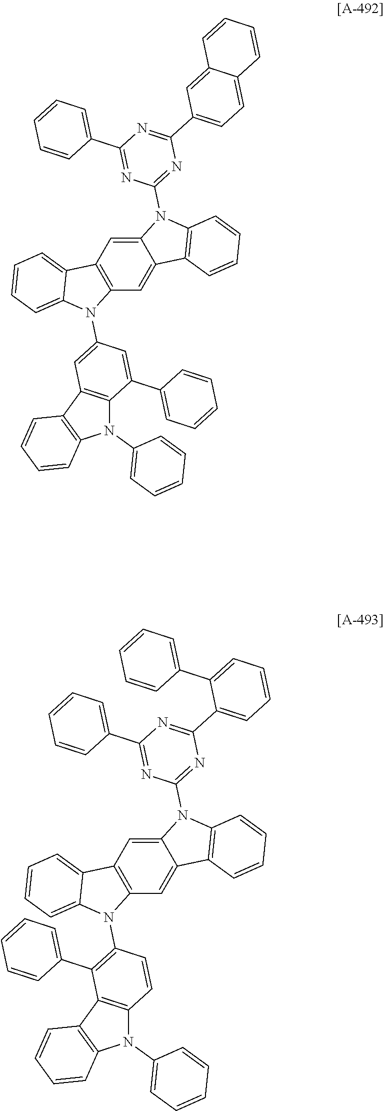 [SMILES: c1ccc(-c2nc(-c3ccc4ccccc4c3)nc(-n3c4ccccc4c4cc5c(cc43)c3ccccc3n5-c3cc(-c4ccccc4)c4c(c3)c3ccccc3n4-c3ccccc3)n2)cc1 ; c1ccc(-c2nc(-c3ccccc3-c3ccccc3)nc(-n3c4ccccc4c4cc5c(cc43)c3ccccc3n5-c3ccc4c(c3-c3ccccc3)c3ccccc3n4-c3ccccc3)n2)cc1]

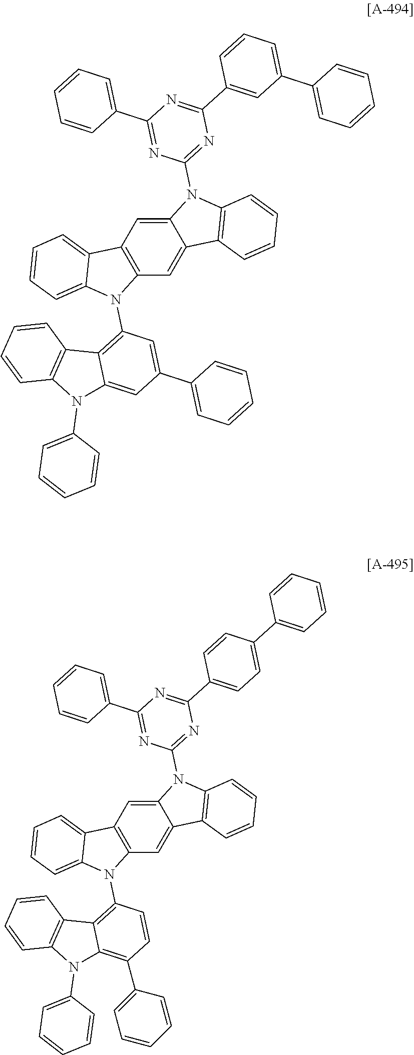 [SMILES: c1ccc(-c2cccc(-c3nc(-c4ccccc4)nc(-n4c5ccccc5c5cc6c(cc54)c4ccccc4n6-c4cc(-c5ccccc5)cc5c4c4ccccc4n5-c4ccccc4)n3)c2)cc1 ; c1ccc(-c2ccc(-c3nc(-c4ccccc4)nc(-n4c5ccccc5c5cc6c(cc54)c4ccccc4n6-c4ccc(-c5ccccc5)c5c4c4ccccc4n5-c4ccccc4)n3)cc2)cc1]

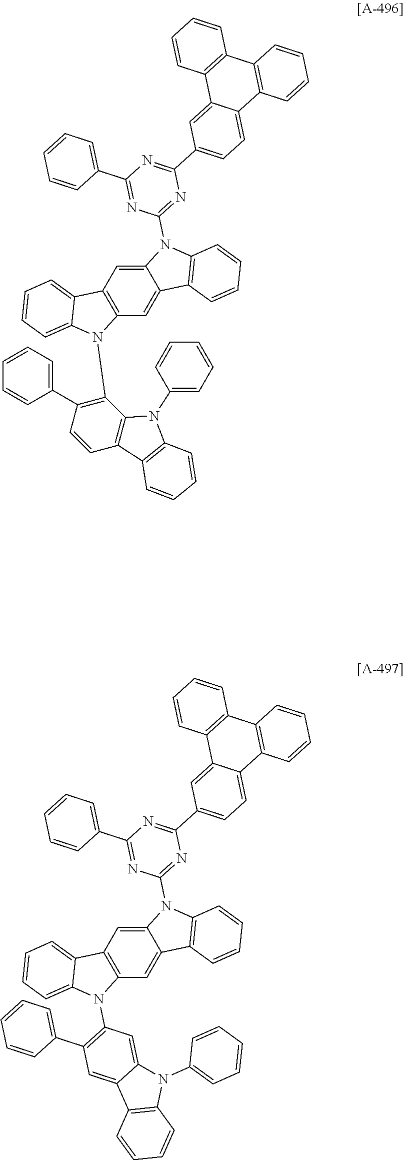 [SMILES: c1ccc(-c2nc(-c3ccc4c5ccccc5c5ccccc5c4c3)nc(-n3c4ccccc4c4cc5c(cc43)c3ccccc3n5-c3c(-c4ccccc4)ccc4c5ccccc5n(-c5ccccc5)c34)n2)cc1 ; c1ccc(-c2nc(-c3ccc4c5ccccc5c5ccccc5c4c3)nc(-n3c4ccccc4c4cc5c(cc43)c3ccccc3n5-c3cc4c(cc3-c3ccccc3)c3ccccc3n4-c3ccccc3)n2)cc1]

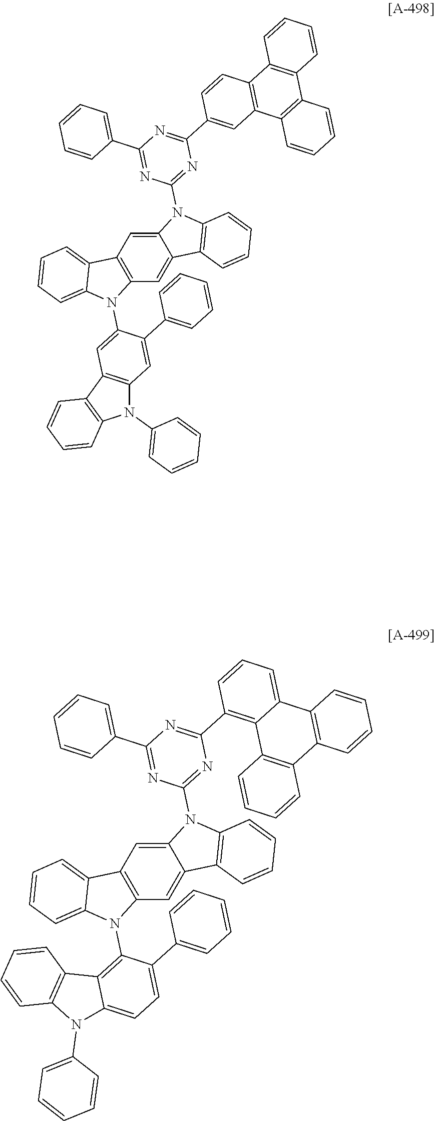 [SMILES: c1ccc(-c2nc(-c3ccc4c5ccccc5c5ccccc5c4c3)nc(-n3c4ccccc4c4cc5c(cc43)c3ccccc3n5-c3cc4c5ccccc5n(-c5ccccc5)c4cc3-c3ccccc3)n2)cc1 ; c1ccc(-c2nc(-c3cccc4c5ccccc5c5ccccc5c34)nc(-n3c4ccccc4c4cc5c(cc43)c3ccccc3n5-c3c(-c4ccccc4)ccc4c3c3ccccc3n4-c3ccccc3)n2)cc1]

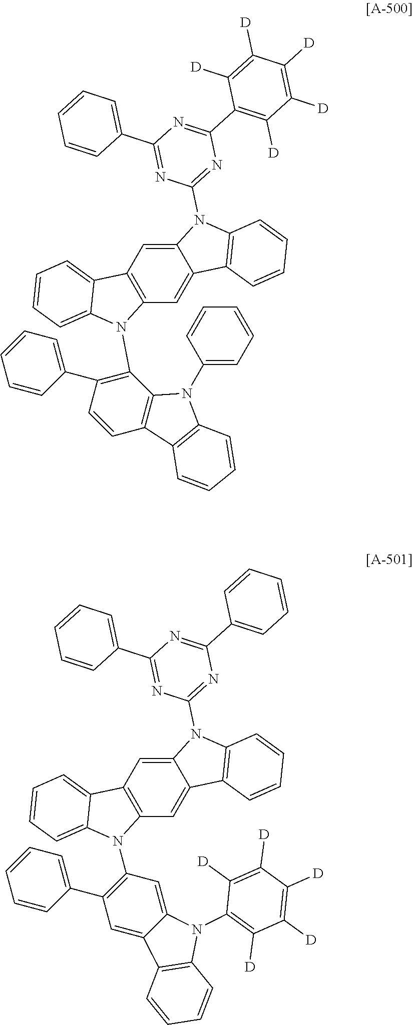 [SMILES: [2H]c1c([2H])c([2H])c(-c2nc(-c3ccccc3)nc(-n3c4ccccc4c4cc5c(cc43)c3ccccc3n5-c3c(-c4ccccc4)ccc4c5ccccc5n(-c5ccccc5)c34)n2)c([2H])c1[2H] ; [2H]c1c([2H])c([2H])c(-n2c3ccccc3c3cc(-c4ccccc4)c(-n4c5ccccc5c5cc6c(cc54)c4ccccc4n6-c4nc(-c5ccccc5)nc(-c5ccccc5)n4)cc32)c([2H])c1[2H]]

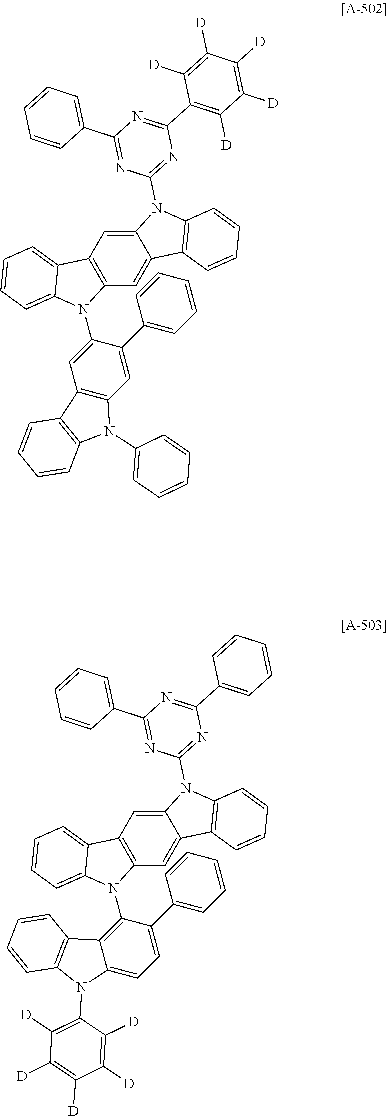 [SMILES: [2H]c1c([2H])c([2H])c(-c2nc(-c3ccccc3)nc(-n3c4ccccc4c4cc5c(cc43)c3ccccc3n5-c3cc4c5ccccc5n(-c5ccccc5)c4cc3-c3ccccc3)n2)c([2H])c1[2H] ; [2H]c1c([2H])c([2H])c(-n2c3ccccc3c3c(-n4c5ccccc5c5cc6c(cc54)c4ccccc4n6-c4nc(-c5ccccc5)nc(-c5ccccc5)n4)c(-c4ccccc4)ccc32)c([2H])c1[2H]]

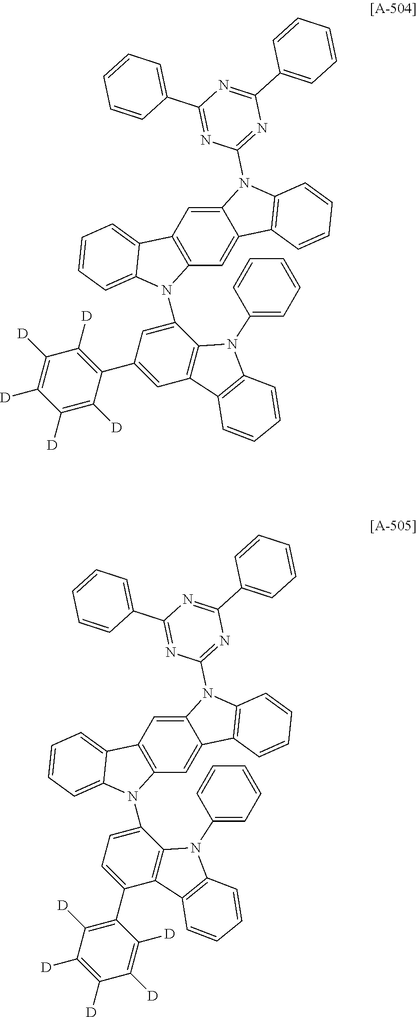 [SMILES: [2H]c1c([2H])c([2H])c(-c2cc(-n3c4ccccc4c4cc5c(cc43)c3ccccc3n5-c3nc(-c4ccccc4)nc(-c4ccccc4)n3)c3c(c2)c2ccccc2n3-c2ccccc2)c([2H])c1[2H] ; [2H]c1c([2H])c([2H])c(-c2ccc(-n3c4ccccc4c4cc5c(cc43)c3ccccc3n5-c3nc(-c4ccccc4)nc(-c4ccccc4)n3)c3c2c2ccccc2n3-c2ccccc2)c([2H])c1[2H]]

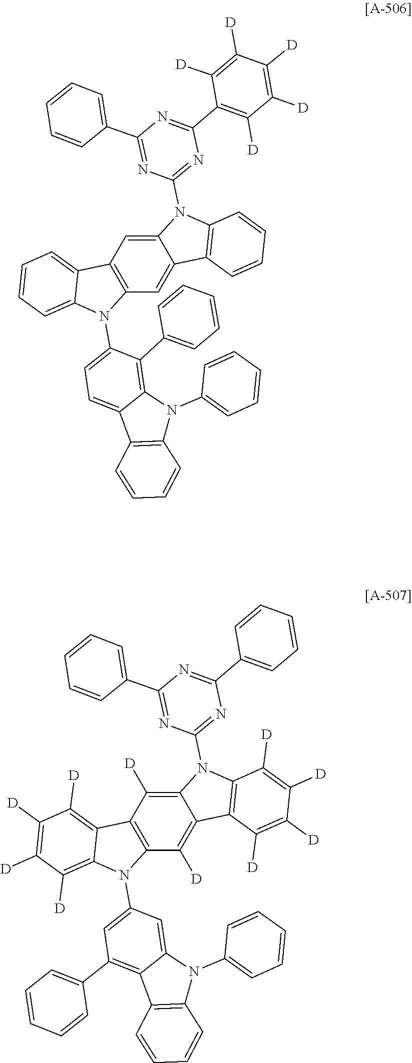 [SMILES: [2H]c1c([2H])c([2H])c(-c2nc(-c3ccccc3)nc(-n3c4ccccc4c4cc5c(cc43)c3ccccc3n5-c3ccc4c5ccccc5n(-c5ccccc5)c4c3-c3ccccc3)n2)c([2H])c1[2H] ; [2H]c1c([2H])c([2H])c2c(c1[2H])c1c([2H])c3c(c([2H])c1n2-c1cc(-c2ccccc2)c2c4ccccc4n(-c4ccccc4)c2c1)c1c([2H])c([2H])c([2H])c([2H])c1n3-c1nc(-c2ccccc2)nc(-c2ccccc2)n1]

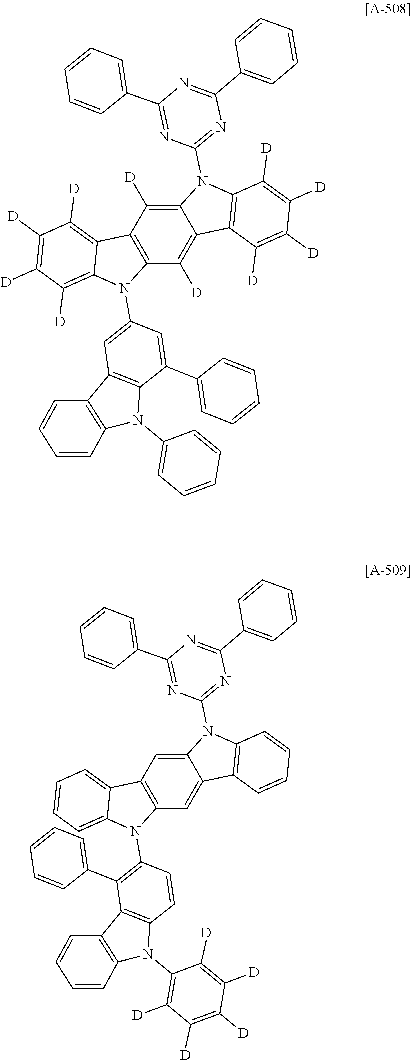 [SMILES: [2H]c1c([2H])c([2H])c2c(c1[2H])c1c([2H])c3c(c([2H])c1n2-c1cc(-c2ccccc2)c2c(c1)c1ccccc1n2-c1ccccc1)c1c([2H])c([2H])c([2H])c([2H])c1n3-c1nc(-c2ccccc2)nc(-c2ccccc2)n1 ; [2H]c1c([2H])c([2H])c(-n2c3ccccc3c3c(-c4ccccc4)c(-n4c5ccccc5c5cc6c(cc54)c4ccccc4n6-c4nc(-c5ccccc5)nc(-c5ccccc5)n4)ccc32)c([2H])c1[2H]]

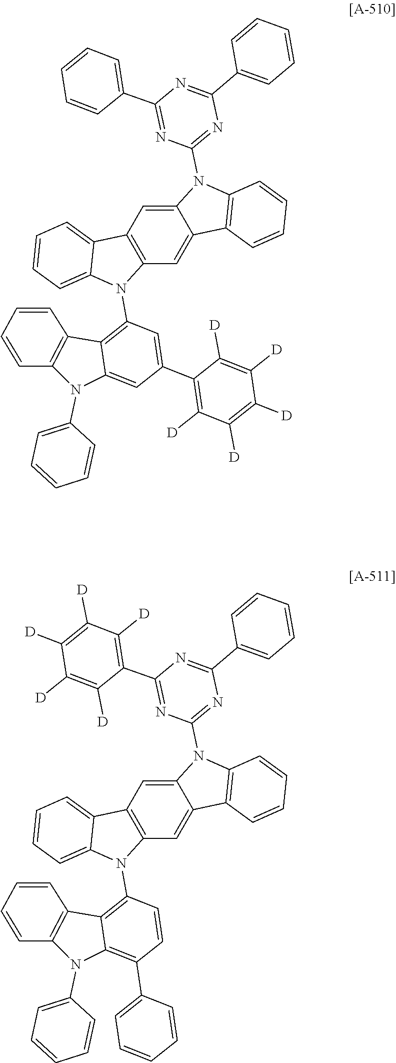 [SMILES: [2H]c1c([2H])c([2H])c(-c2cc(-n3c4ccccc4c4cc5c(cc43)c3ccccc3n5-c3nc(-c4ccccc4)nc(-c4ccccc4)n3)c3c4ccccc4n(-c4ccccc4)c3c2)c([2H])c1[2H] ; [2H]c1c([2H])c([2H])c(-c2nc(-c3ccccc3)nc(-n3c4ccccc4c4cc5c(cc43)c3ccccc3n5-c3ccc(-c4ccccc4)c4c3c3ccccc3n4-c3ccccc3)n2)c([2H])c1[2H]]

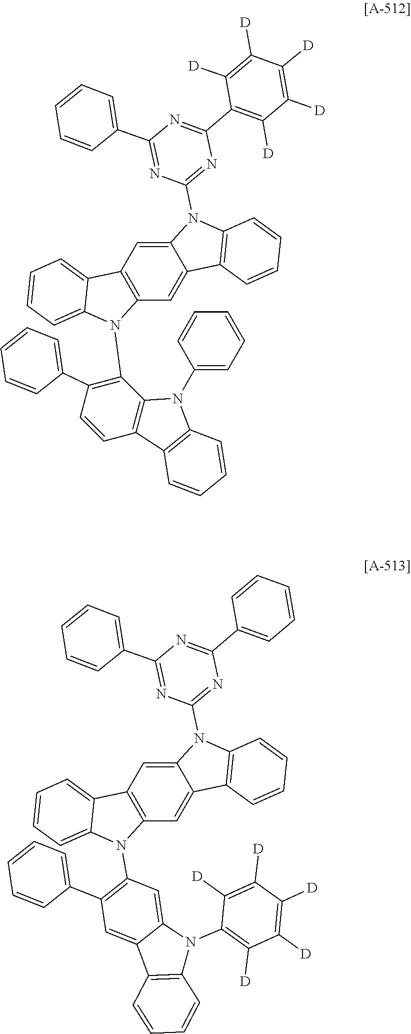 [SMILES: [2H]c1c([2H])c([2H])c(-c2nc(-c3ccccc3)nc(-n3c4ccccc4c4cc5c(cc43)c3ccccc3n5-c3c(-c4ccccc4)ccc4c5ccccc5n(-c5ccccc5)c34)n2)c([2H])c1[2H] ; [2H]c1c([2H])c([2H])c(-n2c3ccccc3c3cc(-c4ccccc4)c(-n4c5ccccc5c5cc6c(cc54)c4ccccc4n6-c4nc(-c5ccccc5)nc(-c5ccccc5)n4)cc32)c([2H])c1[2H]]

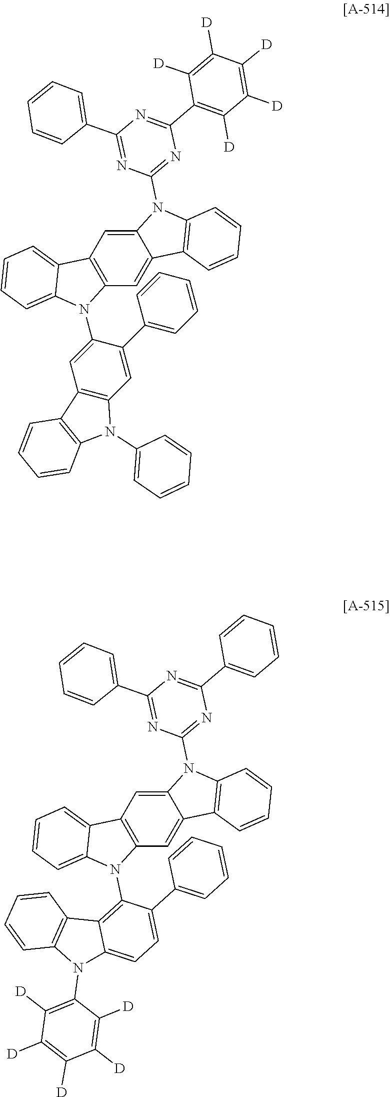 [SMILES: [2H]c1c([2H])c([2H])c(-c2nc(-c3ccccc3)nc(-n3c4ccccc4c4cc5c(cc43)c3ccccc3n5-c3cc4c5ccccc5n(-c5ccccc5)c4cc3-c3ccccc3)n2)c([2H])c1[2H] ; [2H]c1c([2H])c([2H])c(-n2c3ccccc3c3c(-n4c5ccccc5c5cc6c(cc54)c4ccccc4n6-c4nc(-c5ccccc5)nc(-c5ccccc5)n4)c(-c4ccccc4)ccc32)c([2H])c1[2H]]

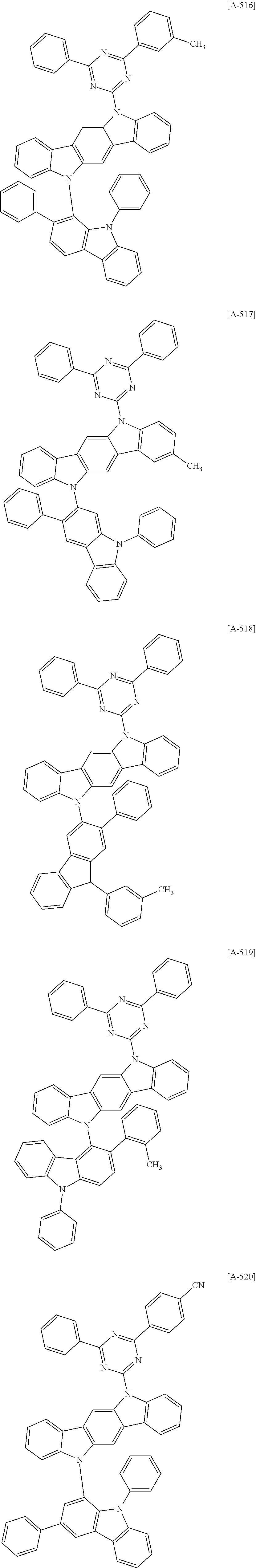

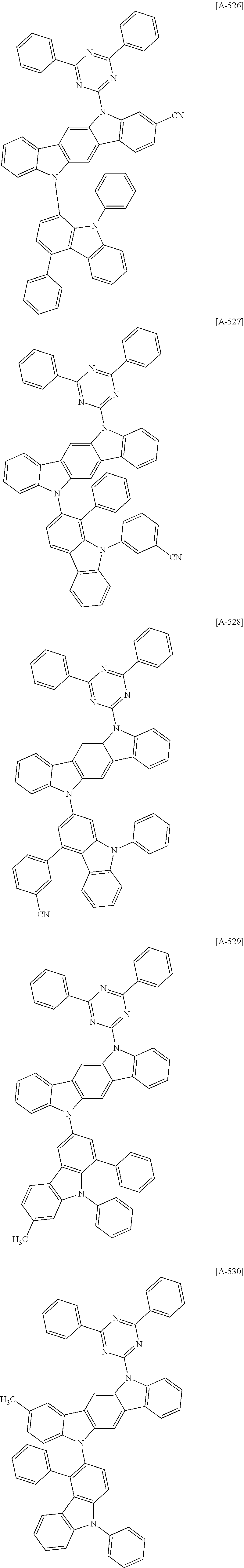

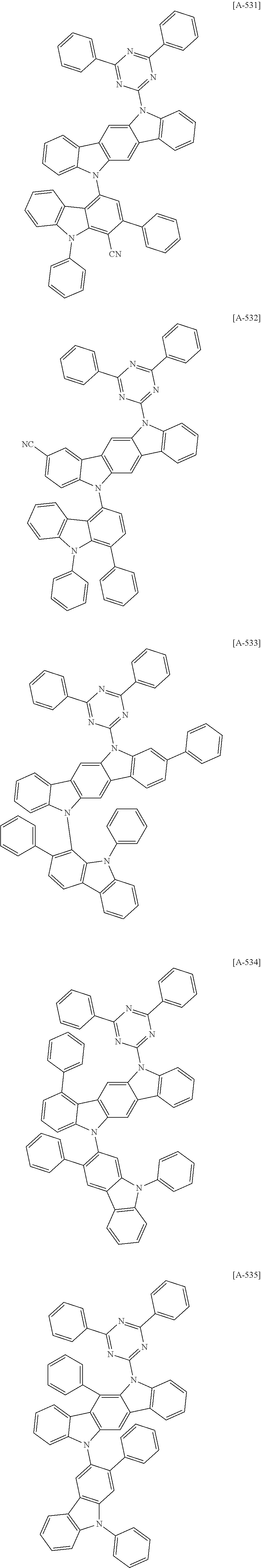

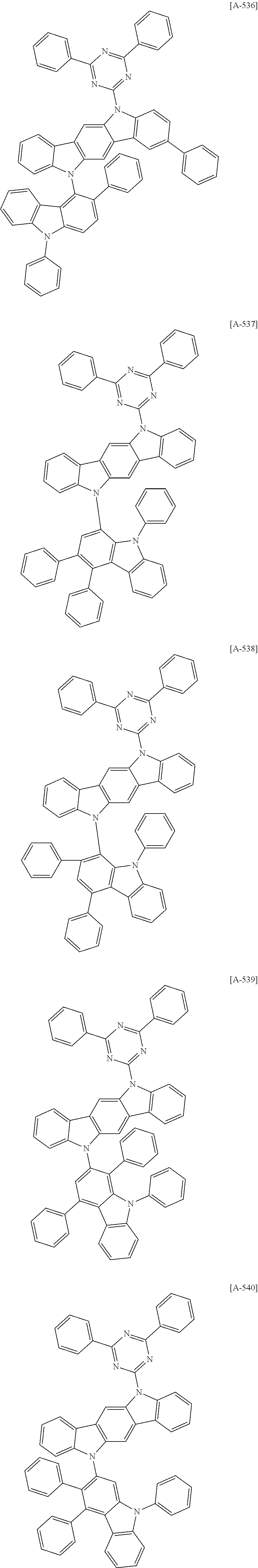

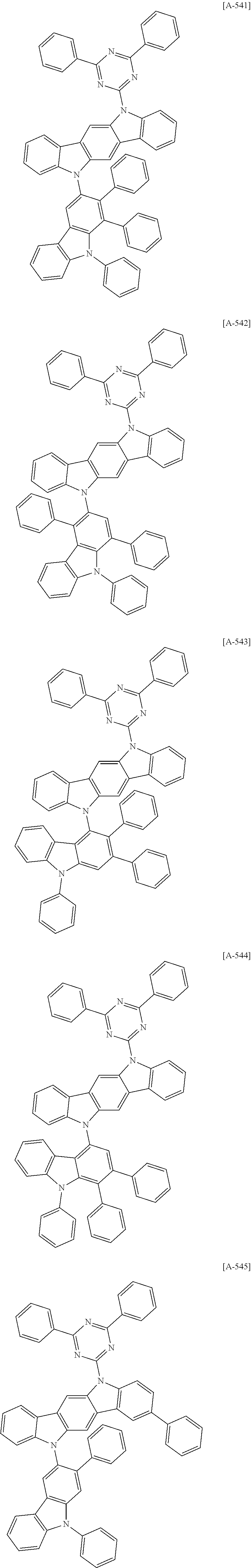

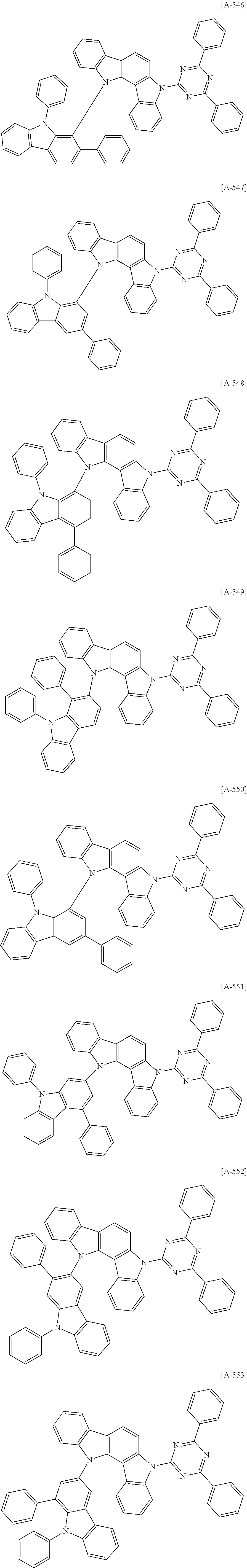

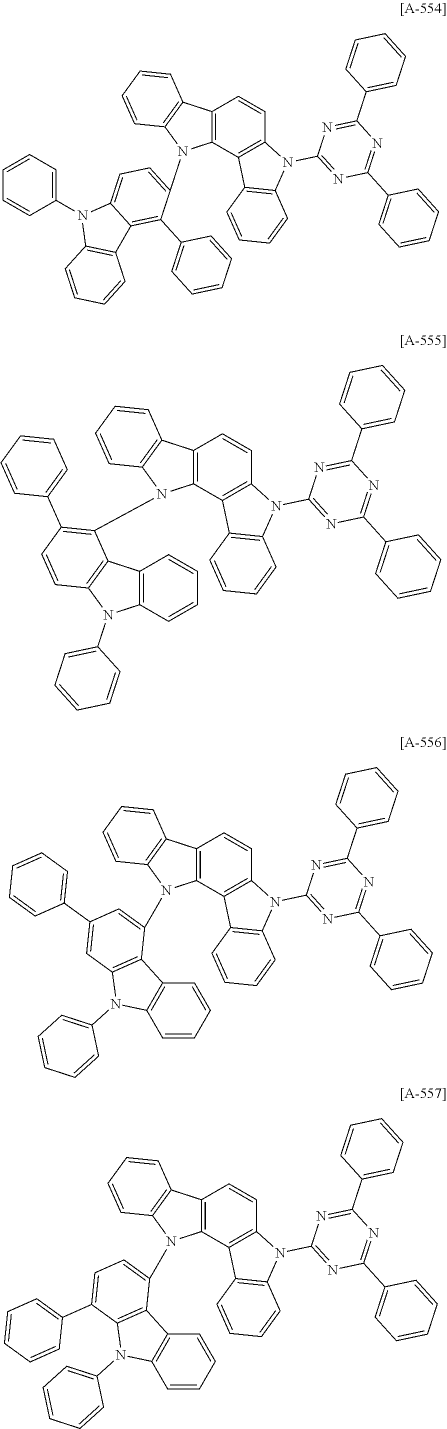 [SMILES: c1ccc(-c2nc(-c3ccccc3)nc(-n3c4ccccc4c4c3ccc3c5ccccc5n(-c5ccc6c(c5-c5ccccc5)c5ccccc5n6-c5ccccc5)c34)n2)cc1 ; c1ccc(-c2nc(-c3ccccc3)nc(-n3c4ccccc4c4c3ccc3c5ccccc5n(-c5c(-c6ccccc6)ccc6c5c5ccccc5n6-c5ccccc5)c34)n2)cc1 ; c1ccc(-c2cc(-n3c4ccccc4c4ccc5c(c6ccccc6n5-c5nc(-c6ccccc6)nc(-c6ccccc6)n5)c43)c3c4ccccc4n(-c4ccccc4)c3c2)cc1 ; c1ccc(-c2nc(-c3ccccc3)nc(-n3c4ccccc4c4c3ccc3c5ccccc5n(-c5ccc(-c6ccccc6)c6c5c5ccccc5n6-c5ccccc5)c34)n2)cc1]

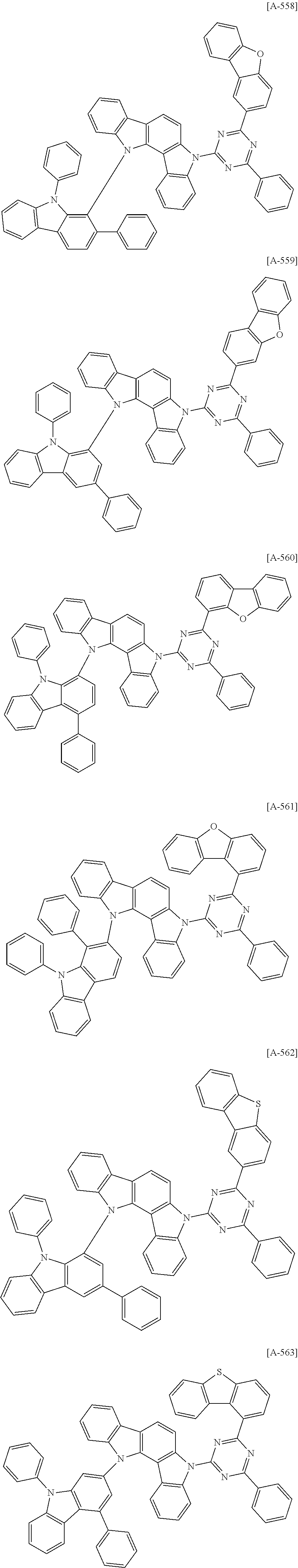

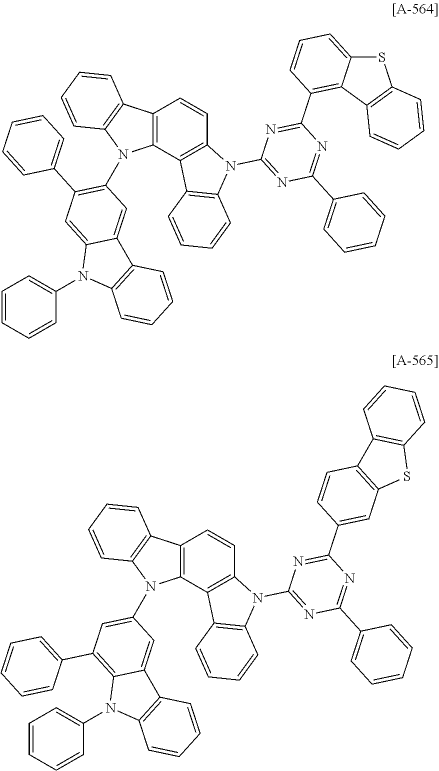 [SMILES: c1ccc(-c2nc(-c3cccc4sc5ccccc5c34)nc(-n3c4ccccc4c4c3ccc3c5ccccc5n(-c5cc6c7ccccc7n(-c7ccccc7)c6cc5-c5ccccc5)c34)n2)cc1 ; c1ccc(-c2nc(-c3ccc4c(c3)sc3ccccc34)nc(-n3c4ccccc4c4c3ccc3c5ccccc5n(-c5cc(-c6ccccc6)c6c(c5)c5ccccc5n6-c5ccccc5)c34)n2)cc1]

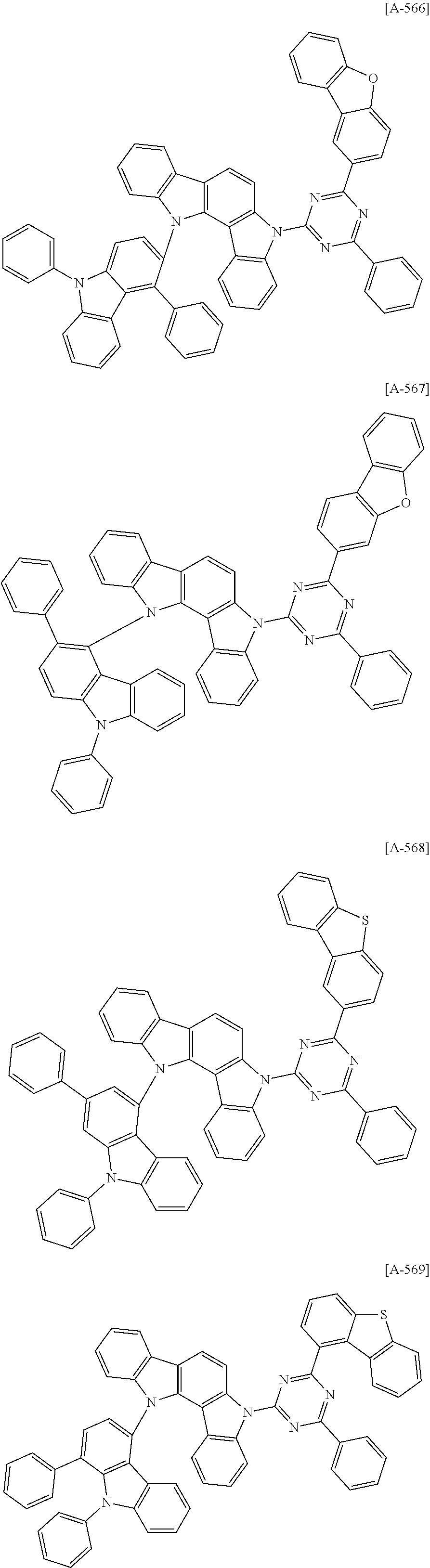 [SMILES: c1ccc(-c2nc(-c3ccc4oc5ccccc5c4c3)nc(-n3c4ccccc4c4c3ccc3c5ccccc5n(-c5ccc6c(c5-c5ccccc5)c5ccccc5n6-c5ccccc5)c34)n2)cc1 ; c1ccc(-c2nc(-c3ccc4c(c3)oc3ccccc34)nc(-n3c4ccccc4c4c3ccc3c5ccccc5n(-c5c(-c6ccccc6)ccc6c5c5ccccc5n6-c5ccccc5)c34)n2)cc1 ; c1ccc(-c2cc(-n3c4ccccc4c4ccc5c(c6ccccc6n5-c5nc(-c6ccccc6)nc(-c6ccc7sc8ccccc8c7c6)n5)c43)c3c4ccccc4n(-c4ccccc4)c3c2)cc1 ; c1ccc(-c2nc(-c3cccc4sc5ccccc5c34)nc(-n3c4ccccc4c4c3ccc3c5ccccc5n(-c5ccc(-c6ccccc6)c6c5c5ccccc5n6-c5ccccc5)c34)n2)cc1]

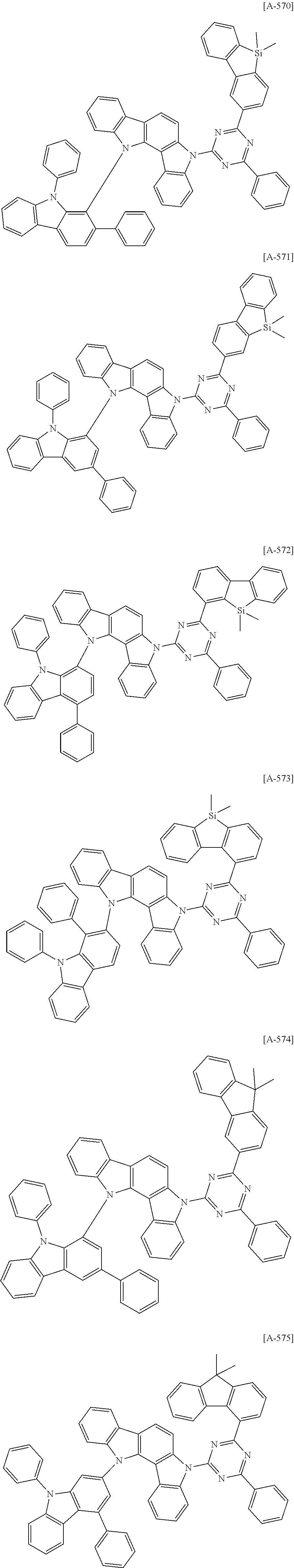

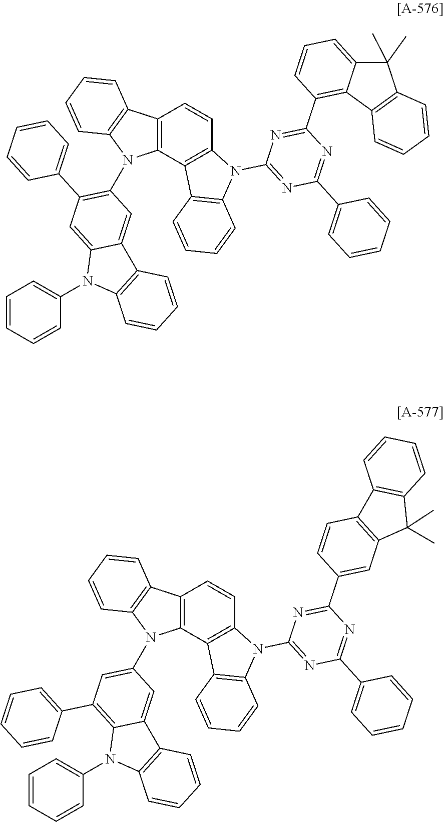 [SMILES: CC1(C)c2ccccc2-c2c(-c3nc(-c4ccccc4)nc(-n4c5ccccc5c5c4ccc4c6ccccc6n(-c6cc7c8ccccc8n(-c8ccccc8)c7cc6-c6ccccc6)c45)n3)cccc21 ; CC1(C)c2ccccc2-c2ccc(-c3nc(-c4ccccc4)nc(-n4c5ccccc5c5c4ccc4c6ccccc6n(-c6cc(-c7ccccc7)c7c(c6)c6ccccc6n7-c6ccccc6)c45)n3)cc21]

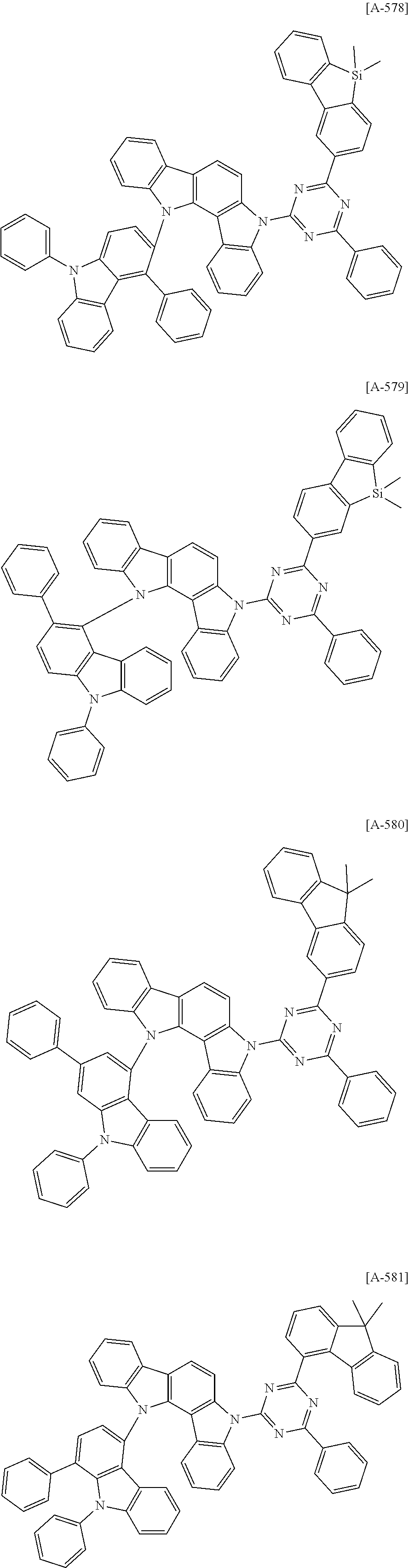 [SMILES: C[Si]1(C)c2ccccc2-c2cc(-c3nc(-c4ccccc4)nc(-n4c5ccccc5c5c4ccc4c6ccccc6n(-c6ccc7c(c6-c6ccccc6)c6ccccc6n7-c6ccccc6)c45)n3)ccc21 ; C[Si]1(C)c2ccccc2-c2ccc(-c3nc(-c4ccccc4)nc(-n4c5ccccc5c5c4ccc4c6ccccc6n(-c6c(-c7ccccc7)ccc7c6c6ccccc6n7-c6ccccc6)c45)n3)cc21 ; CC1(C)c2ccccc2-c2cc(-c3nc(-c4ccccc4)nc(-n4c5ccccc5c5c4ccc4c6ccccc6n(-c6cc(-c7ccccc7)cc7c6c6ccccc6n7-c6ccccc6)c45)n3)ccc21 ; CC1(C)c2ccccc2-c2c(-c3nc(-c4ccccc4)nc(-n4c5ccccc5c5c4ccc4c6ccccc6n(-c6ccc(-c7ccccc7)c7c6c6ccccc6n7-c6ccccc6)c45)n3)cccc21]

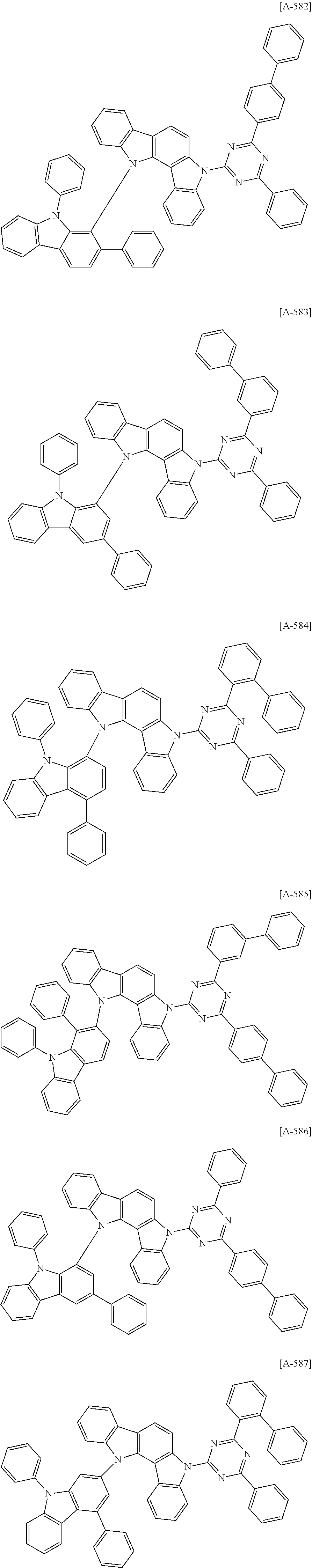

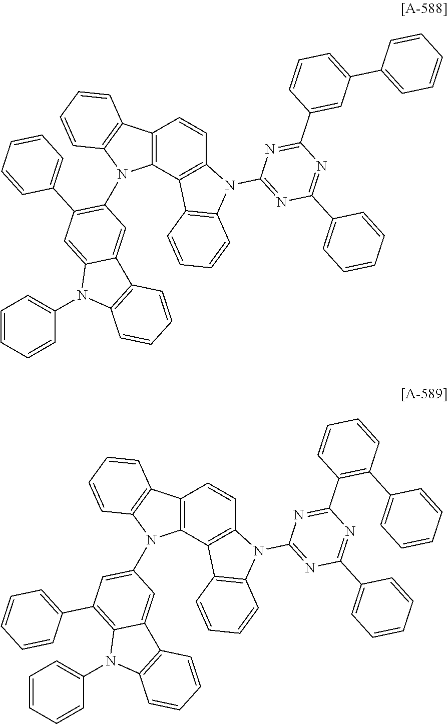 [SMILES: c1ccc(-c2cccc(-c3nc(-c4ccccc4)nc(-n4c5ccccc5c5c4ccc4c6ccccc6n(-c6cc7c8ccccc8n(-c8ccccc8)c7cc6-c6ccccc6)c45)n3)c2)cc1 ; c1ccc(-c2nc(-c3ccccc3-c3ccccc3)nc(-n3c4ccccc4c4c3ccc3c5ccccc5n(-c5cc(-c6ccccc6)c6c(c5)c5ccccc5n6-c5ccccc5)c34)n2)cc1]

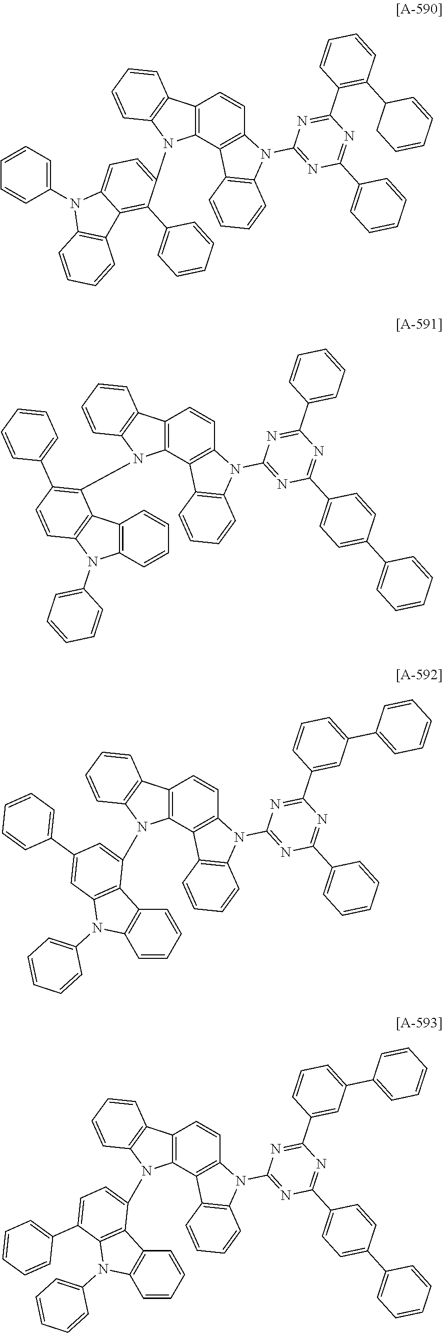 [SMILES: C1=CCC(c2ccccc2-c2nc(-c3ccccc3)nc(-n3c4ccccc4c4c3ccc3c5ccccc5n(-c5ccc6c(c5-c5ccccc5)c5ccccc5n6-c5ccccc5)c34)n2)C=C1 ; c1ccc(-c2ccc(-c3nc(-c4ccccc4)nc(-n4c5ccccc5c5c4ccc4c6ccccc6n(-c6c(-c7ccccc7)ccc7c6c6ccccc6n7-c6ccccc6)c45)n3)cc2)cc1 ; c1ccc(-c2cccc(-c3nc(-c4ccccc4)nc(-n4c5ccccc5c5c4ccc4c6ccccc6n(-c6cc(-c7ccccc7)cc7c6c6ccccc6n7-c6ccccc6)c45)n3)c2)cc1 ; c1ccc(-c2ccc(-c3nc(-c4cccc(-c5ccccc5)c4)nc(-n4c5ccccc5c5c4ccc4c6ccccc6n(-c6ccc(-c7ccccc7)c7c6c6ccccc6n7-c6ccccc6)c45)n3)cc2)cc1]

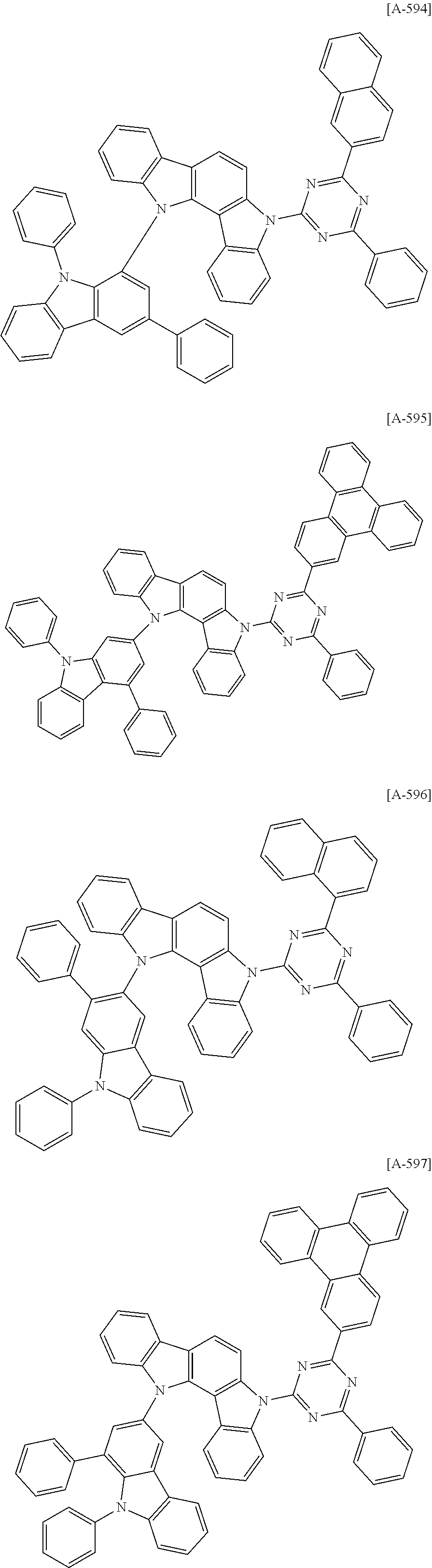 [SMILES: c1ccc(-c2cc(-n3c4ccccc4c4ccc5c(c6ccccc6n5-c5nc(-c6ccccc6)nc(-c6ccc7ccccc7c6)n5)c43)c3c(c2)c2ccccc2n3-c2ccccc2)cc1 ; c1ccc(-c2nc(-c3ccc4c5ccccc5c5ccccc5c4c3)nc(-n3c4ccccc4c4c3ccc3c5ccccc5n(-c5cc(-c6ccccc6)c6c7ccccc7n(-c7ccccc7)c6c5)c34)n2)cc1 ; c1ccc(-c2nc(-c3cccc4ccccc34)nc(-n3c4ccccc4c4c3ccc3c5ccccc5n(-c5cc6c7ccccc7n(-c7ccccc7)c6cc5-c5ccccc5)c34)n2)cc1 ; c1ccc(-c2nc(-c3ccc4c5ccccc5c5ccccc5c4c3)nc(-n3c4ccccc4c4c3ccc3c5ccccc5n(-c5cc(-c6ccccc6)c6c(c5)c5ccccc5n6-c5ccccc5)c34)n2)cc1]

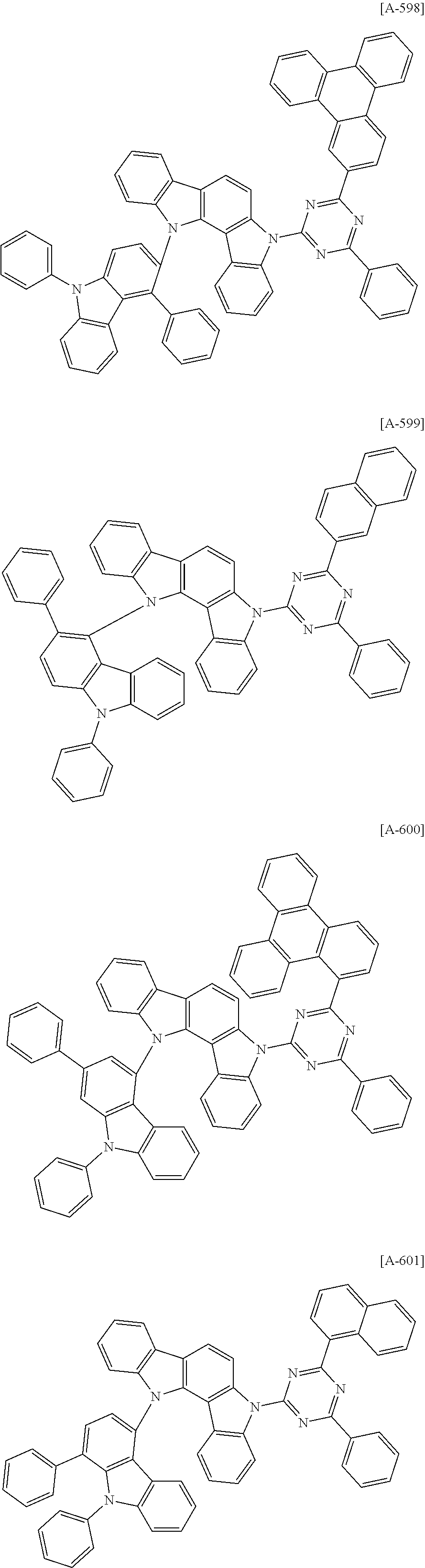 [SMILES: c1ccc(-c2nc(-c3ccc4c5ccccc5c5ccccc5c4c3)nc(-n3c4ccccc4c4c3ccc3c5ccccc5n(-c5ccc6c(c5-c5ccccc5)c5ccccc5n6-c5ccccc5)c34)n2)cc1 ; c1ccc(-c2nc(-c3ccc4ccccc4c3)nc(-n3c4ccccc4c4c3ccc3c5ccccc5n(-c5c(-c6ccccc6)ccc6c5c5ccccc5n6-c5ccccc5)c34)n2)cc1 ; c1ccc(-c2cc(-n3c4ccccc4c4ccc5c(c6ccccc6n5-c5nc(-c6ccccc6)nc(-c6cccc7c8ccccc8c8ccccc8c67)n5)c43)c3c4ccccc4n(-c4ccccc4)c3c2)cc1 ; c1ccc(-c2nc(-c3cccc4ccccc34)nc(-n3c4ccccc4c4c3ccc3c5ccccc5n(-c5ccc(-c6ccccc6)c6c5c5ccccc5n6-c5ccccc5)c34)n2)cc1]

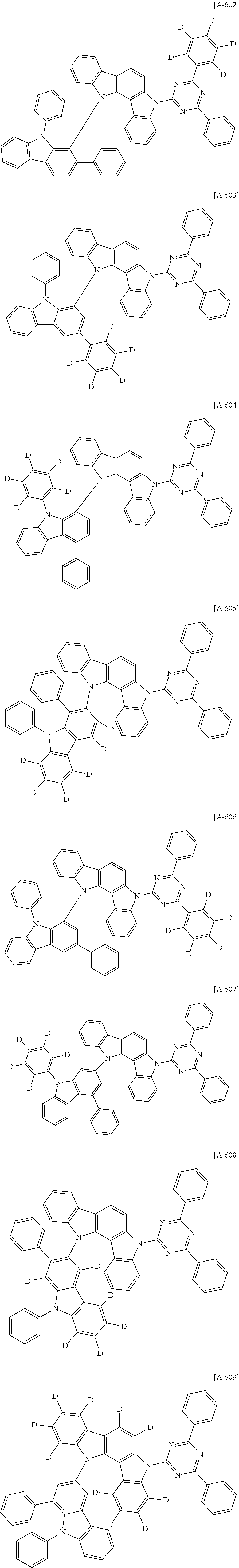

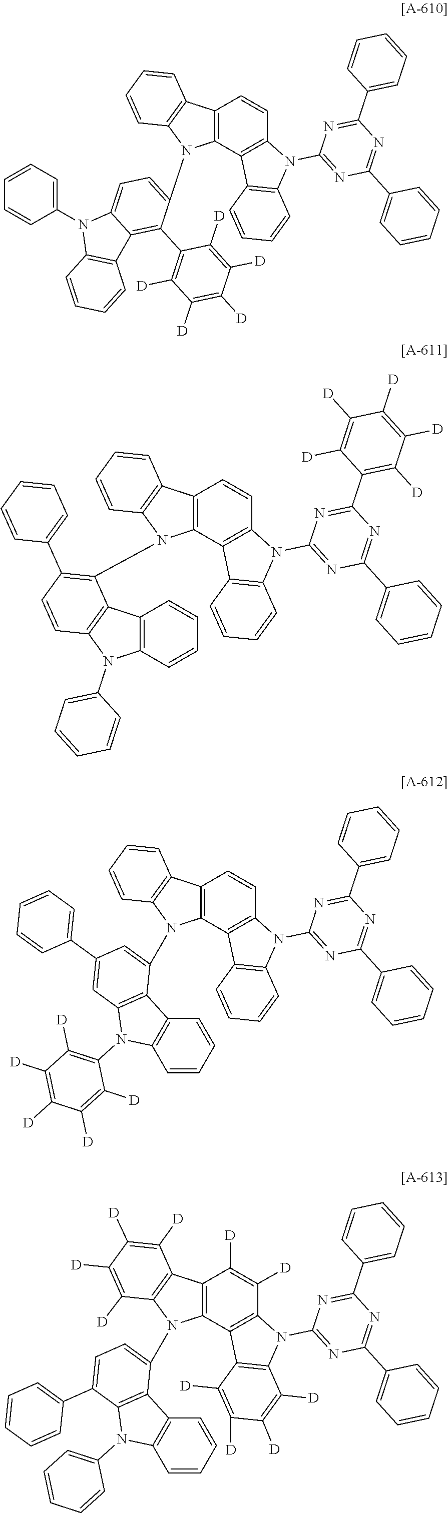

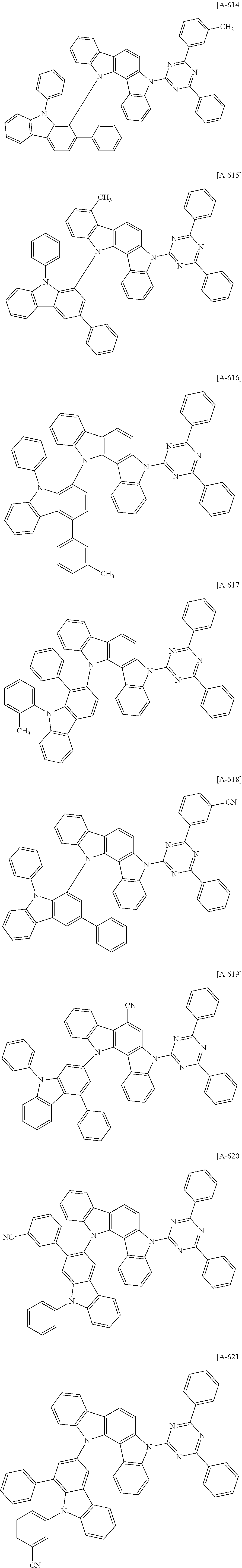

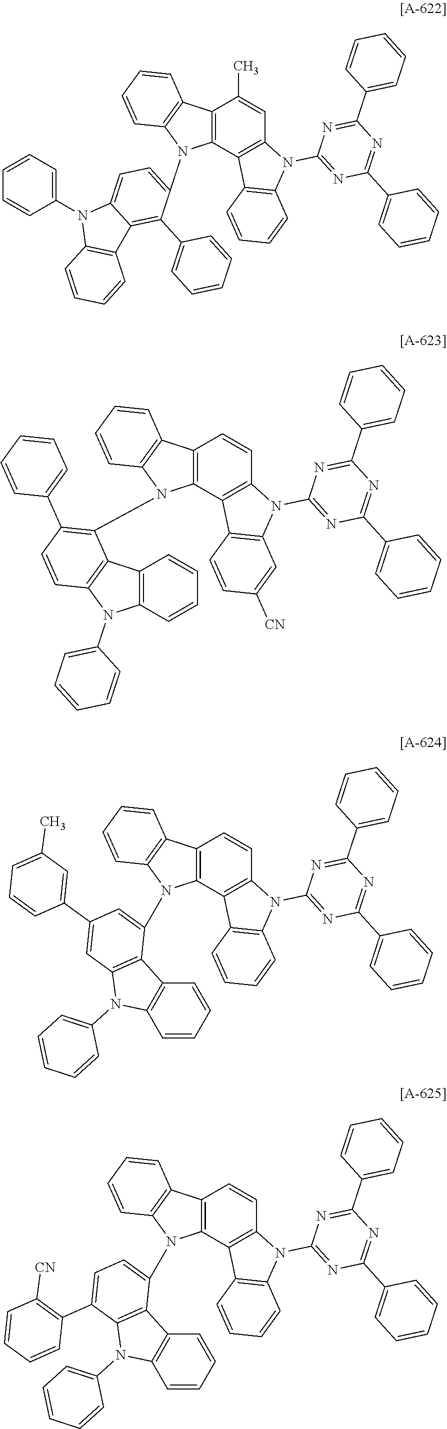 [SMILES: Cc1cc2c(c3ccccc3n2-c2nc(-c3ccccc3)nc(-c3ccccc3)n2)c2c1c1ccccc1n2-c1ccc2c(c1-c1ccccc1)c1ccccc1n2-c1ccccc1 ; N#Cc1ccc2c3c(ccc4c5ccccc5n(-c5c(-c6ccccc6)ccc6c5c5ccccc5n6-c5ccccc5)c43)n(-c3nc(-c4ccccc4)nc(-c4ccccc4)n3)c2c1 ; Cc1cccc(-c2cc(-n3c4ccccc4c4ccc5c(c6ccccc6n5-c5nc(-c6ccccc6)nc(-c6ccccc6)n5)c43)c3c4ccccc4n(-c4ccccc4)c3c2)c1 ; N#Cc1ccccc1-c1ccc(-n2c3ccccc3c3ccc4c(c5ccccc5n4-c4nc(-c5ccccc5)nc(-c5ccccc5)n4)c32)c2c3ccccc3n(-c3ccccc3)c12]

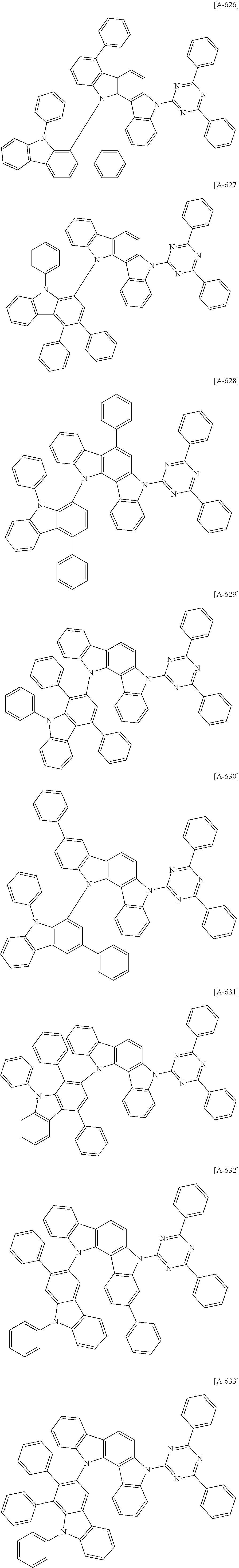

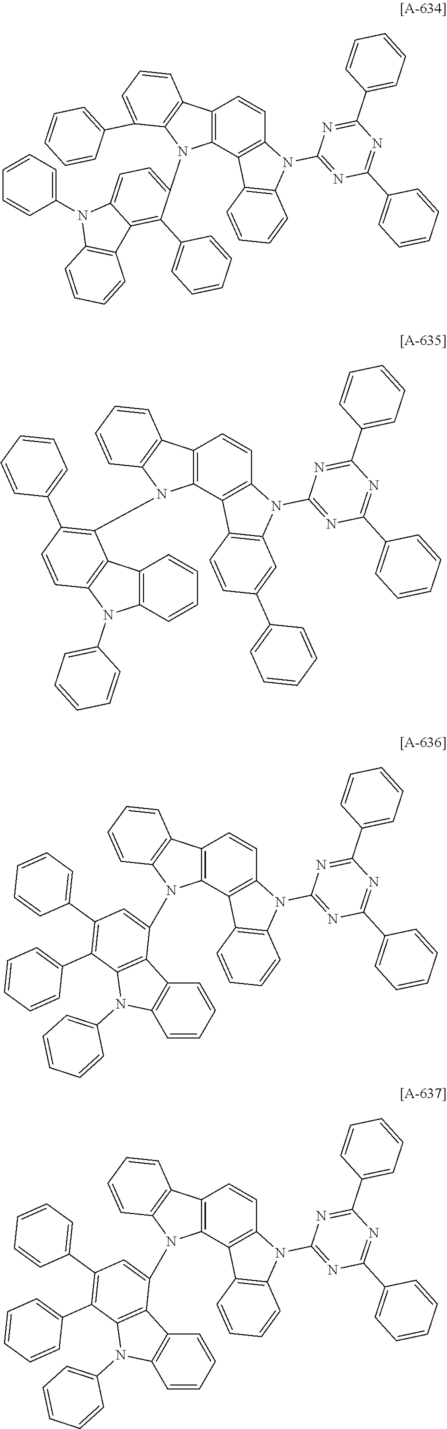 [SMILES: c1ccc(-c2nc(-c3ccccc3)nc(-n3c4ccccc4c4c3ccc3c5cccc(-c6ccccc6)c5n(-c5ccc6c(c5-c5ccccc5)c5ccccc5n6-c5ccccc5)c34)n2)cc1 ; c1ccc(-c2ccc3c4c(ccc5c6ccccc6n(-c6c(-c7ccccc7)ccc7c6c6ccccc6n7-c6ccccc6)c54)n(-c4nc(-c5ccccc5)nc(-c5ccccc5)n4)c3c2)cc1 ; c1ccc(-c2nc(-c3ccccc3)nc(-n3c4ccccc4c4c3ccc3c5ccccc5n(-c5cc(-c6ccccc6)c(-c6ccccc6)c6c5c5ccccc5n6-c5ccccc5)c34)n2)cc1 ; c1ccc(-c2nc(-c3ccccc3)nc(-n3c4ccccc4c4c3ccc3c5ccccc5n(-c5cc(-c6ccccc6)c(-c6ccccc6)c6c5c5ccccc5n6-c5ccccc5)c34)n2)cc1]

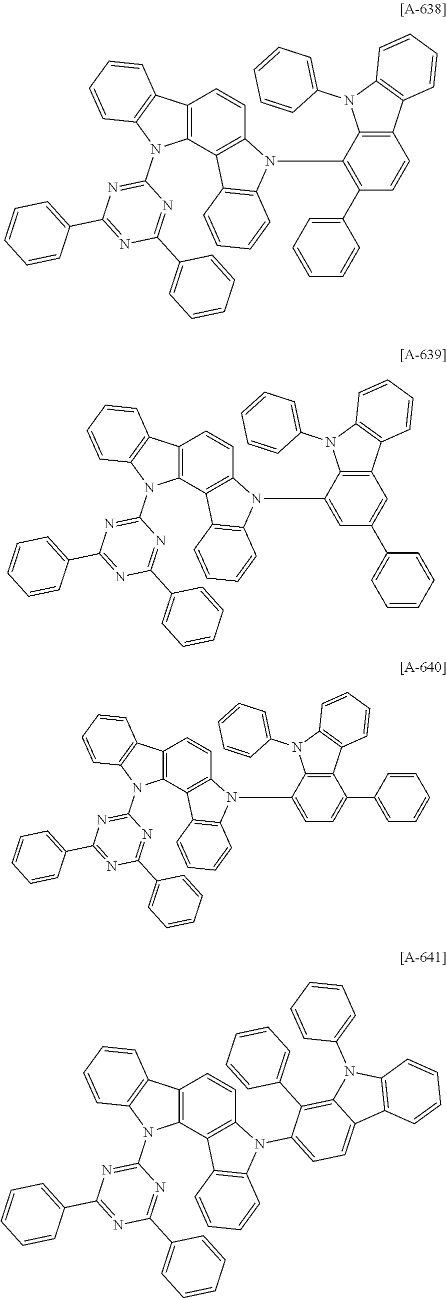 [SMILES: c1ccc(-c2nc(-c3ccccc3)nc(-n3c4ccccc4c4ccc5c(c6ccccc6n5Cc5c(-c6ccccc6)ccc6c7ccccc7n(-c7ccccc7)c56)c43)n2)cc1 ; c1ccc(-c2cc(Cn3c4ccccc4c4c3ccc3c5ccccc5n(-c5nc(-c6ccccc6)nc(-c6ccccc6)n5)c34)c3c(c2)c2ccccc2n3-c2ccccc2)cc1 ; c1ccc(-c2nc(-c3ccccc3)nc(-n3c4ccccc4c4ccc5c(c6ccccc6n5Cc5ccc(-c6ccccc6)c6c7ccccc7n(-c7ccccc7)c56)c43)n2)cc1 ; c1ccc(-c2nc(-c3ccccc3)nc(-n3c4ccccc4c4ccc5c(c6ccccc6n5-c5ccc6c7ccccc7n(-c7ccccc7)c6c5-c5ccccc5)c43)n2)cc1]

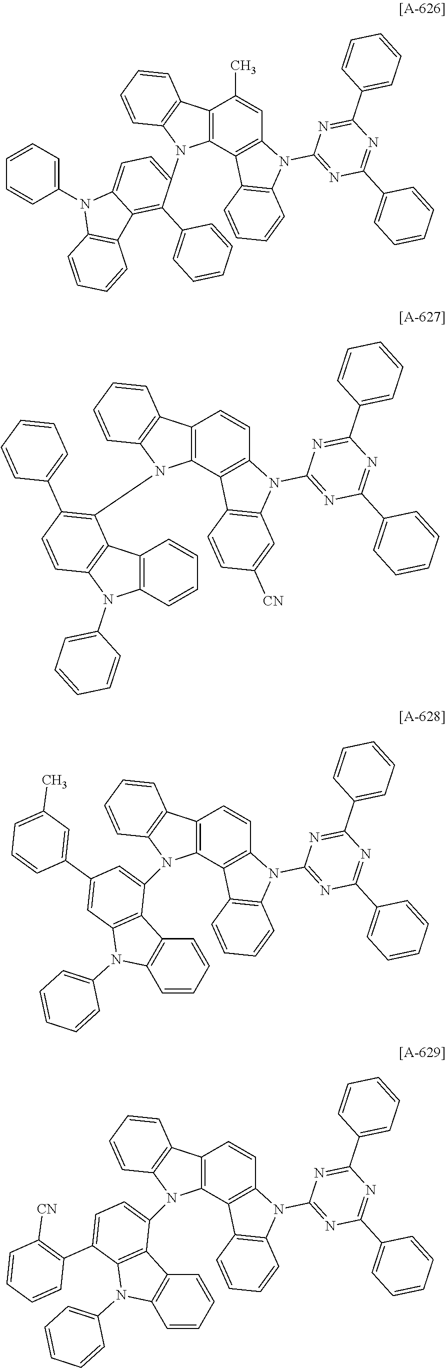 [SMILES: Cc1cc2c(c3ccccc3n2-c2nc(-c3ccccc3)nc(-c3ccccc3)n2)c2c1c1ccccc1n2-c1ccc2c(c1-c1ccccc1)c1ccccc1n2-c1ccccc1 ; N#Cc1ccc2c3c(ccc4c5ccccc5n(-c5c(-c6ccccc6)ccc6c5c5ccccc5n6-c5ccccc5)c43)n(-c3nc(-c4ccccc4)nc(-c4ccccc4)n3)c2c1 ; Cc1cccc(-c2cc(-n3c4ccccc4c4ccc5c(c6ccccc6n5-c5nc(-c6ccccc6)nc(-c6ccccc6)n5)c43)c3c4ccccc4n(-c4ccccc4)c3c2)c1 ; N#Cc1ccccc1-c1ccc(-n2c3ccccc3c3ccc4c(c5ccccc5n4-c4nc(-c5ccccc5)nc(-c5ccccc5)n4)c32)c2c3ccccc3n(-c3ccccc3)c12]

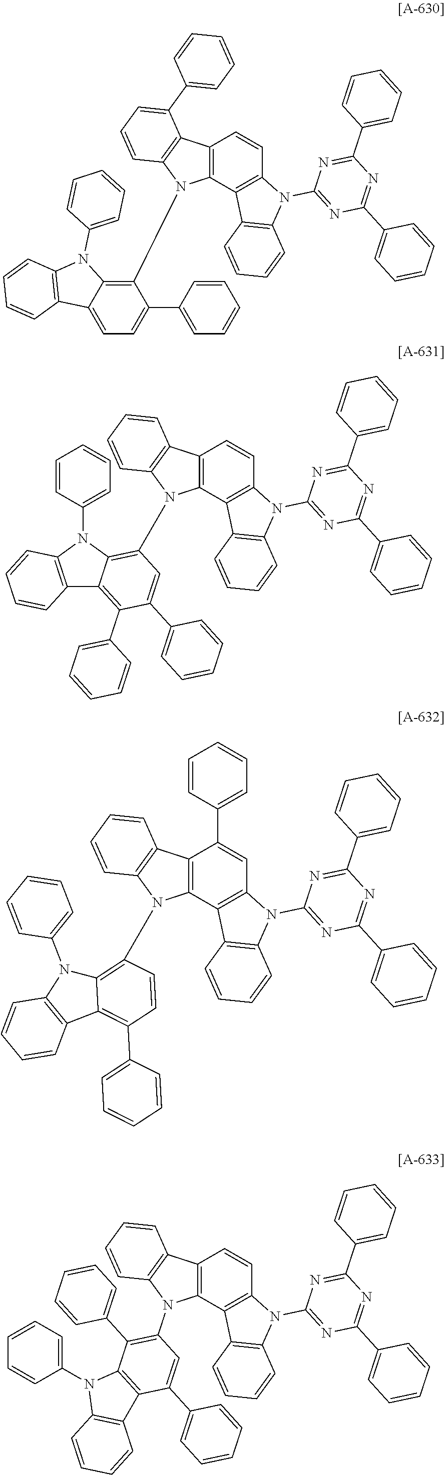 [SMILES: c1ccc(-c2nc(-c3ccccc3)nc(-n3c4ccccc4c4c3ccc3c5c(-c6ccccc6)cccc5n(-c5c(-c6ccccc6)ccc6c7ccccc7n(-c7ccccc7)c56)c34)n2)cc1 ; c1ccc(-c2nc(-c3ccccc3)nc(-n3c4ccccc4c4c3ccc3c5ccccc5n(-c5cc(-c6ccccc6)c(-c6ccccc6)c6c7ccccc7n(-c7ccccc7)c56)c34)n2)cc1 ; c1ccc(-c2nc(-c3ccccc3)nc(-n3c4ccccc4c4c3cc(-c3ccccc3)c3c5ccccc5n(-c5ccc(-c6ccccc6)c6c7ccccc7n(-c7ccccc7)c56)c34)n2)cc1 ; c1ccc(-c2nc(-c3ccccc3)nc(-n3c4ccccc4c4c3ccc3c5ccccc5n(-c5cc(-c6ccccc6)c6c7ccccc7n(-c7ccccc7)c6c5-c5ccccc5)c34)n2)cc1]

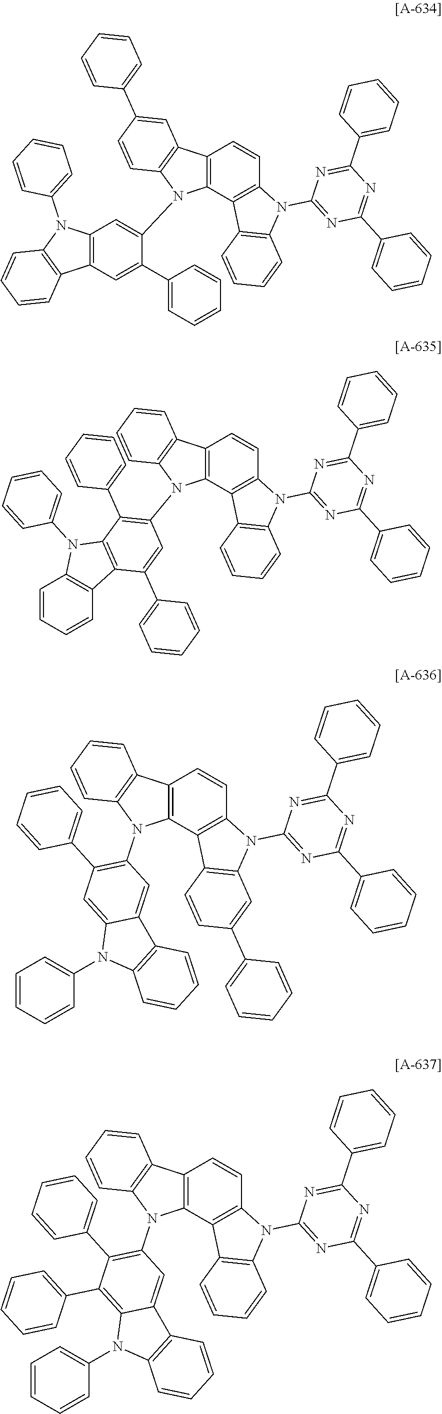 [SMILES: c1ccc(-c2ccc3c(c2)c2ccc4c(c5ccccc5n4-c4nc(-c5ccccc5)nc(-c5ccccc5)n4)c2n3-c2cc3c(cc2-c2ccccc2)c2ccccc2n3-c2ccccc2)cc1 ; c1ccc(-c2nc(-c3ccccc3)nc(-n3c4ccccc4c4c3ccc3c5ccccc5n(-c5cc(-c6ccccc6)c6c7ccccc7n(-c7ccccc7)c6c5-c5ccccc5)c34)n2)cc1 ; c1ccc(-c2ccc3c4c(ccc5c6ccccc6n(-c6cc7c8ccccc8n(-c8ccccc8)c7cc6-c6ccccc6)c54)n(-c4nc(-c5ccccc5)nc(-c5ccccc5)n4)c3c2)cc1 ; c1ccc(-c2nc(-c3ccccc3)nc(-n3c4ccccc4c4c3ccc3c5ccccc5n(-c5cc6c7ccccc7n(-c7ccccc7)c6c(-c6ccccc6)c5-c5ccccc5)c34)n2)cc1]

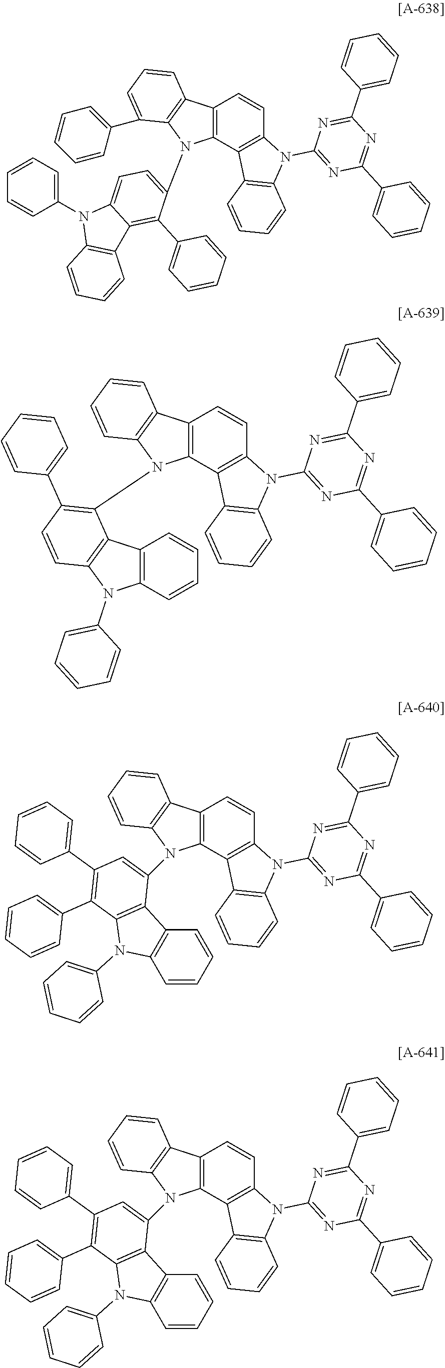 [SMILES: c1ccc(-c2nc(-c3ccccc3)nc(-n3c4ccccc4c4c3ccc3c5cccc(-c6ccccc6)c5n(-c5ccc6c(c5-c5ccccc5)c5ccccc5n6-c5ccccc5)c34)n2)cc1 ; c1ccc(-c2nc(-c3ccccc3)nc(-n3c4ccccc4c4c3ccc3c5ccccc5n(-c5c(-c6ccccc6)ccc6c5c5ccccc5n6-c5ccccc5)c34)n2)cc1 ; c1ccc(-c2nc(-c3ccccc3)nc(-n3c4ccccc4c4c3ccc3c5ccccc5n(-c5cc(-c6ccccc6)c(-c6ccccc6)c6c5c5ccccc5n6-c5ccccc5)c34)n2)cc1 ; c1ccc(-c2nc(-c3ccccc3)nc(-n3c4ccccc4c4c3ccc3c5ccccc5n(-c5cc(-c6ccccc6)c(-c6ccccc6)c6c5c5ccccc5n6-c5ccccc5)c34)n2)cc1]

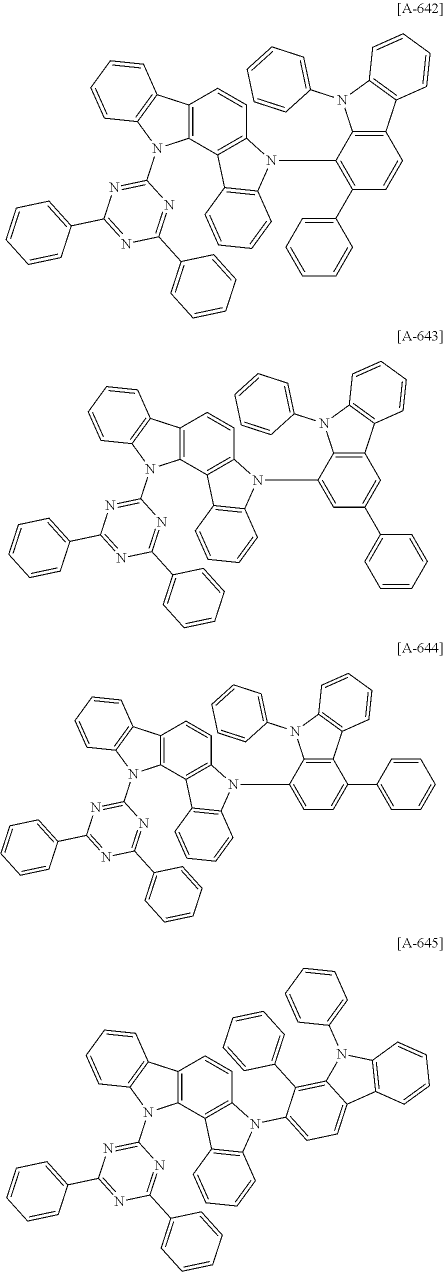 [SMILES: c1ccc(-c2nc(-c3ccccc3)nc(-n3c4ccccc4c4ccc5c(c6ccccc6n5-c5c(-c6ccccc6)ccc6c7ccccc7n(-c7ccccc7)c56)c43)n2)cc1 ; c1ccc(-c2cc(-n3c4ccccc4c4c3ccc3c5ccccc5n(-c5nc(-c6ccccc6)nc(-c6ccccc6)n5)c34)c3c(c2)c2ccccc2n3-c2ccccc2)cc1 ; c1ccc(-c2nc(-c3ccccc3)nc(-n3c4ccccc4c4ccc5c(c6ccccc6n5-c5ccc(-c6ccccc6)c6c7ccccc7n(-c7ccccc7)c56)c43)n2)cc1 ; c1ccc(-c2nc(-c3ccccc3)nc(-n3c4ccccc4c4ccc5c(c6ccccc6n5-c5ccc6c7ccccc7n(-c7ccccc7)c6c5-c5ccccc5)c43)n2)cc1]

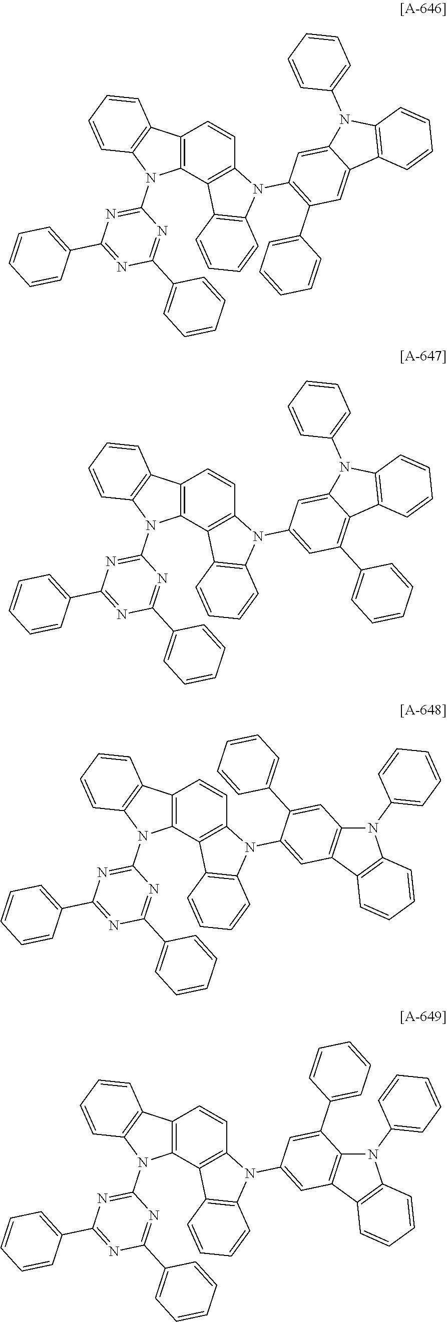 [SMILES: c1ccc(-c2nc(-c3ccccc3)nc(-n3c4ccccc4c4ccc5c(c6ccccc6n5-c5cc6c(cc5-c5ccccc5)c5ccccc5n6-c5ccccc5)c43)n2)cc1 ; c1ccc(-c2nc(-c3ccccc3)nc(-n3c4ccccc4c4ccc5c(c6ccccc6n5-c5cc(-c6ccccc6)c6c7ccccc7n(-c7ccccc7)c6c5)c43)n2)cc1 ; c1ccc(-c2nc(-c3ccccc3)nc(-n3c4ccccc4c4ccc5c(c6ccccc6n5-c5cc6c7ccccc7n(-c7ccccc7)c6cc5-c5ccccc5)c43)n2)cc1 ; c1ccc(-c2nc(-c3ccccc3)nc(-n3c4ccccc4c4ccc5c(c6ccccc6n5-c5cc(-c6ccccc6)c6c(c5)c5ccccc5n6-c5ccccc5)c43)n2)cc1]

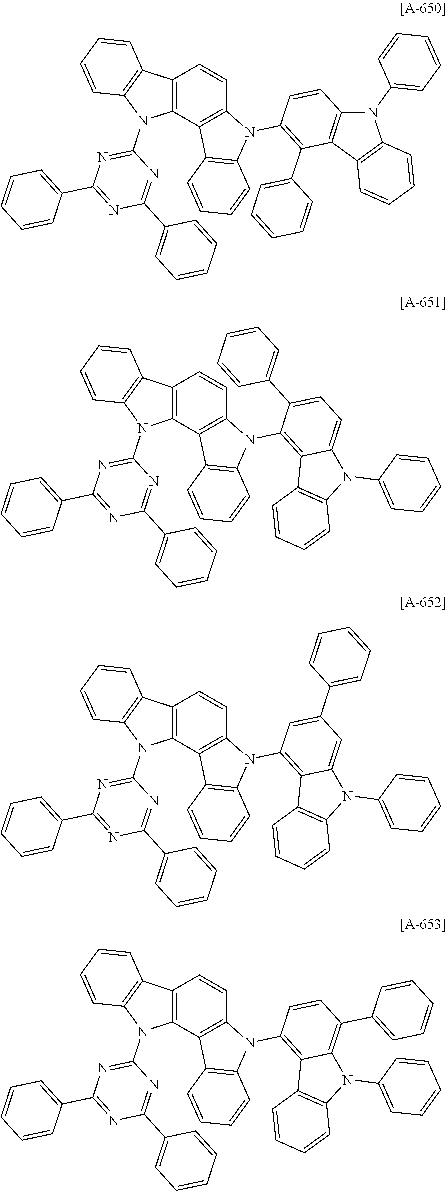 [SMILES: c1ccc(-c2nc(-c3ccccc3)nc(-n3c4ccccc4c4ccc5c(c6ccccc6n5-c5ccc6c(c5-c5ccccc5)c5ccccc5n6-c5ccccc5)c43)n2)cc1 ; c1ccc(-c2nc(-c3ccccc3)nc(-n3c4ccccc4c4ccc5c(c6ccccc6n5-c5c(-c6ccccc6)ccc6c5c5ccccc5n6-c5ccccc5)c43)n2)cc1 ; c1ccc(-c2cc(-n3c4ccccc4c4c3ccc3c5ccccc5n(-c5nc(-c6ccccc6)nc(-c6ccccc6)n5)c34)c3c4ccccc4n(-c4ccccc4)c3c2)cc1 ; c1ccc(-c2nc(-c3ccccc3)nc(-n3c4ccccc4c4ccc5c(c6ccccc6n5-c5ccc(-c6ccccc6)c6c5c5ccccc5n6-c5ccccc5)c43)n2)cc1]

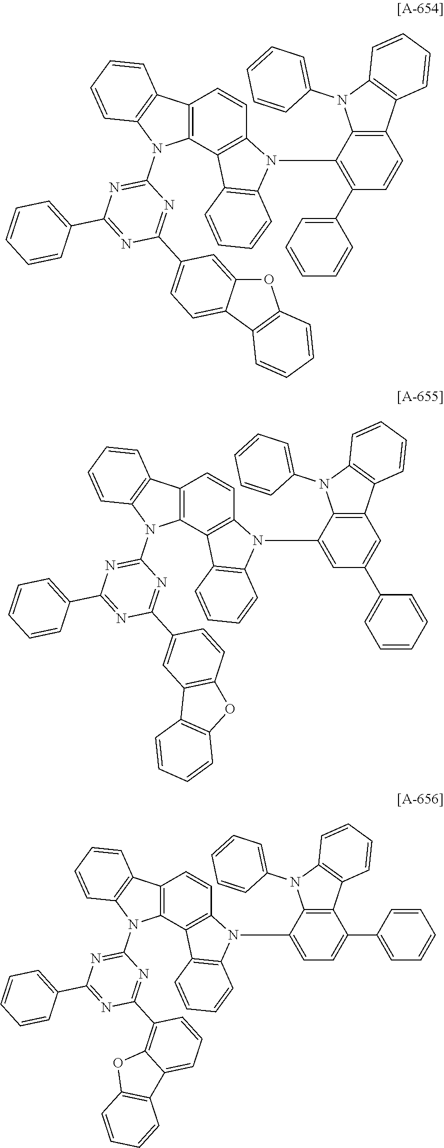 [SMILES: c1ccc(-c2nc(-c3ccc4c(c3)oc3ccccc34)nc(-n3c4ccccc4c4ccc5c(c6ccccc6n5-c5c(-c6ccccc6)ccc6c7ccccc7n(-c7ccccc7)c56)c43)n2)cc1 ; c1ccc(-c2cc(-n3c4ccccc4c4c3ccc3c5ccccc5n(-c5nc(-c6ccccc6)nc(-c6ccc7oc8ccccc8c7c6)n5)c34)c3c(c2)c2ccccc2n3-c2ccccc2)cc1 ; c1ccc(-c2nc(-c3cccc4c3oc3ccccc34)nc(-n3c4ccccc4c4ccc5c(c6ccccc6n5-c5ccc(-c6ccccc6)c6c7ccccc7n(-c7ccccc7)c56)c43)n2)cc1]

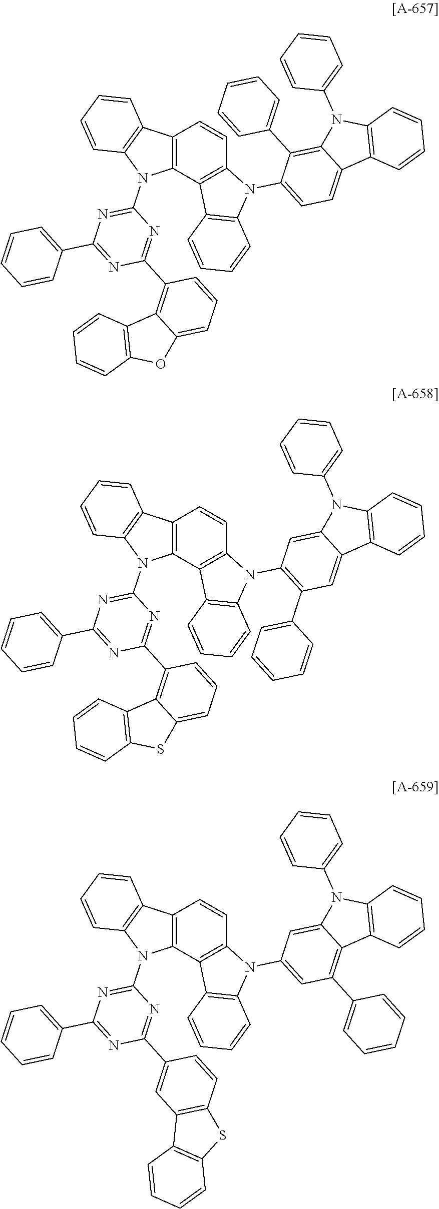 [SMILES: c1ccc(-c2nc(-c3cccc4oc5ccccc5c34)nc(-n3c4ccccc4c4ccc5c(c6ccccc6n5-c5ccc6c7ccccc7n(-c7ccccc7)c6c5-c5ccccc5)c43)n2)cc1 ; c1ccc(-c2nc(-c3cccc4sc5ccccc5c34)nc(-n3c4ccccc4c4ccc5c(c6ccccc6n5-c5cc6c(cc5-c5ccccc5)c5ccccc5n6-c5ccccc5)c43)n2)cc1 ; c1ccc(-c2nc(-c3ccc4sc5ccccc5c4c3)nc(-n3c4ccccc4c4ccc5c(c6ccccc6n5-c5cc(-c6ccccc6)c6c7ccccc7n(-c7ccccc7)c6c5)c43)n2)cc1]

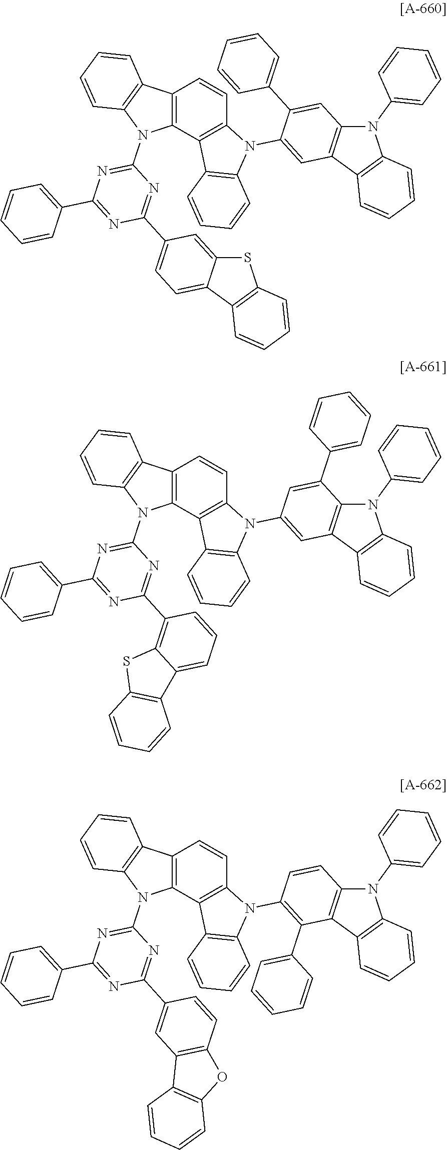 [SMILES: c1ccc(-c2nc(-c3ccc4c(c3)sc3ccccc34)nc(-n3c4ccccc4c4ccc5c(c6ccccc6n5-c5cc6c7ccccc7n(-c7ccccc7)c6cc5-c5ccccc5)c43)n2)cc1 ; c1ccc(-c2nc(-c3cccc4c3sc3ccccc34)nc(-n3c4ccccc4c4ccc5c(c6ccccc6n5-c5cc(-c6ccccc6)c6c(c5)c5ccccc5n6-c5ccccc5)c43)n2)cc1 ; c1ccc(-c2nc(-c3ccc4oc5ccccc5c4c3)nc(-n3c4ccccc4c4ccc5c(c6ccccc6n5-c5ccc6c(c5-c5ccccc5)c5ccccc5n6-c5ccccc5)c43)n2)cc1]

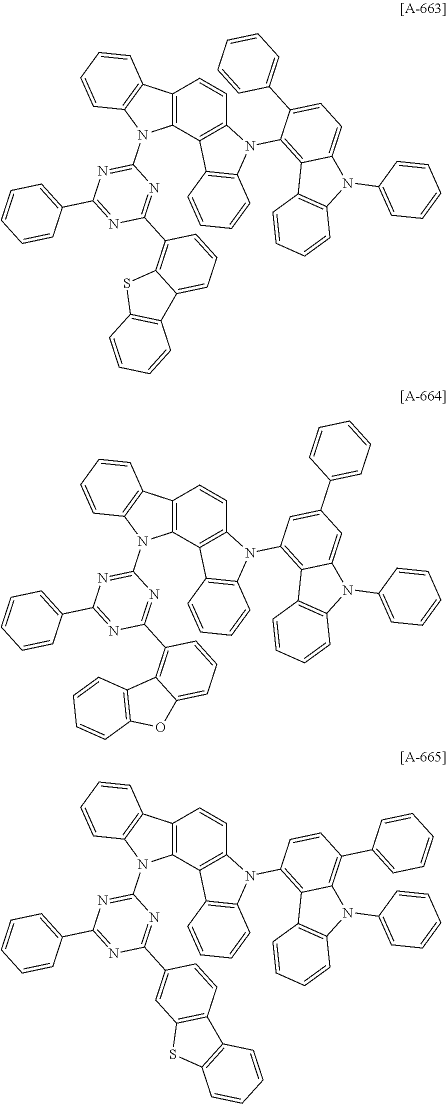

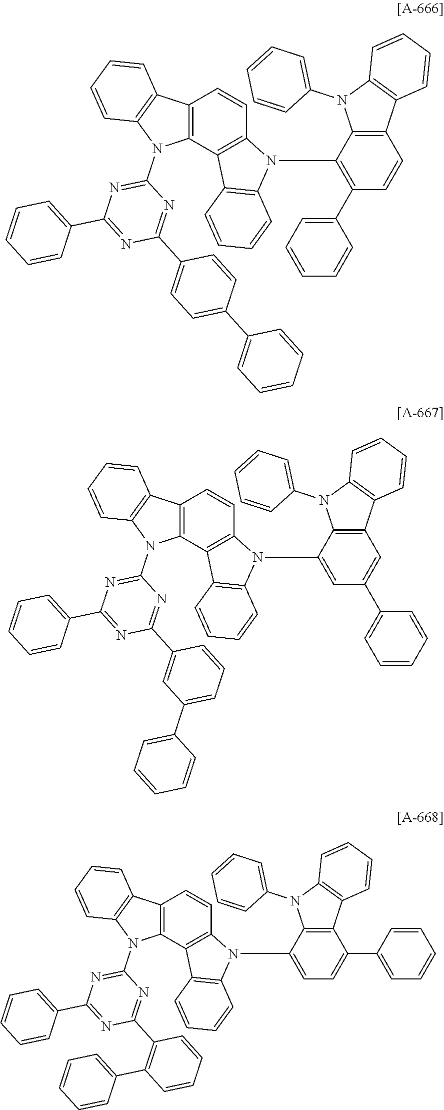 [SMILES: c1ccc(-c2ccc(-c3nc(-c4ccccc4)nc(-n4c5ccccc5c5ccc6c(c7ccccc7n6-c6c(-c7ccccc7)ccc7c8ccccc8n(-c8ccccc8)c67)c54)n3)cc2)cc1 ; c1ccc(-c2cccc(-c3nc(-c4ccccc4)nc(-n4c5ccccc5c5ccc6c(c7ccccc7n6-c6cc(-c7ccccc7)cc7c8ccccc8n(-c8ccccc8)c67)c54)n3)c2)cc1 ; c1ccc(-c2nc(-c3ccccc3-c3ccccc3)nc(-n3c4ccccc4c4ccc5c(c6ccccc6n5-c5ccc(-c6ccccc6)c6c7ccccc7n(-c7ccccc7)c56)c43)n2)cc1]

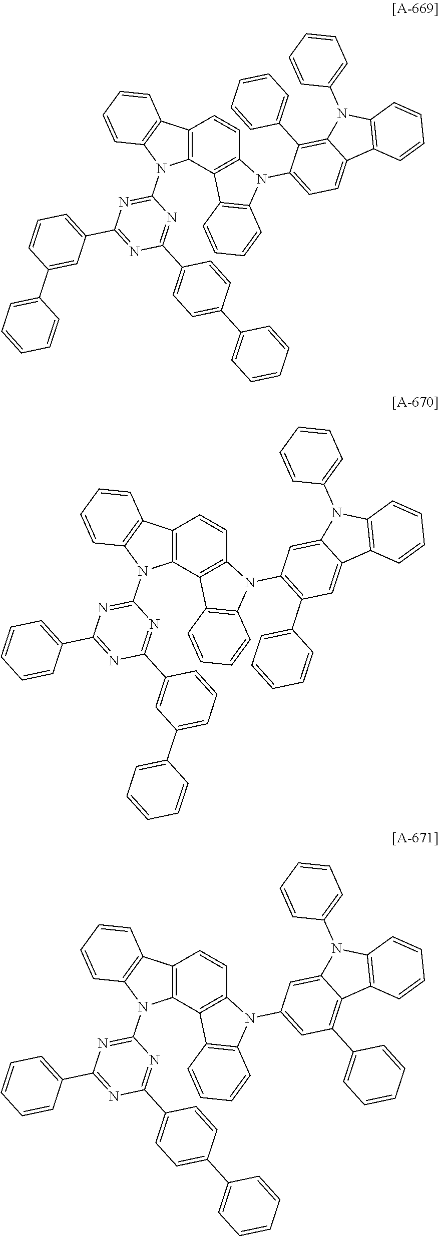 [SMILES: c1ccc(-c2ccc(-c3nc(-c4cccc(-c5ccccc5)c4)nc(-n4c5ccccc5c5ccc6c(c7ccccc7n6-c6ccc7c8ccccc8n(-c8ccccc8)c7c6-c6ccccc6)c54)n3)cc2)cc1 ; c1ccc(-c2cccc(-c3nc(-c4ccccc4)nc(-n4c5ccccc5c5ccc6c(c7ccccc7n6-c6cc7c(cc6-c6ccccc6)c6ccccc6n7-c6ccccc6)c54)n3)c2)cc1 ; c1ccc(-c2ccc(-c3nc(-c4ccccc4)nc(-n4c5ccccc5c5ccc6c(c7ccccc7n6-c6cc(-c7ccccc7)c7c8ccccc8n(-c8ccccc8)c7c6)c54)n3)cc2)cc1]

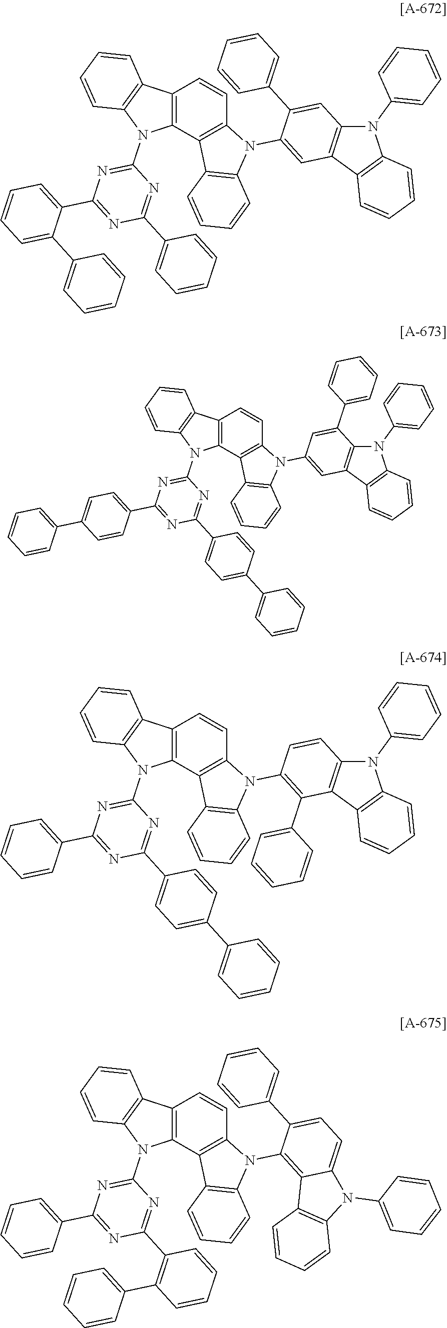 [SMILES: c1ccc(-c2nc(-c3ccccc3-c3ccccc3)nc(-n3c4ccccc4c4ccc5c(c6ccccc6n5-c5cc6c7ccccc7n(-c7ccccc7)c6cc5-c5ccccc5)c43)n2)cc1 ; c1ccc(-c2ccc(-c3nc(-c4ccc(-c5ccccc5)cc4)nc(-n4c5ccccc5c5ccc6c(c7ccccc7n6-c6cc(-c7ccccc7)c7c(c6)c6ccccc6n7-c6ccccc6)c54)n3)cc2)cc1 ; c1ccc(-c2ccc(-c3nc(-c4ccccc4)nc(-n4c5ccccc5c5ccc6c(c7ccccc7n6-c6ccc7c(c6-c6ccccc6)c6ccccc6n7-c6ccccc6)c54)n3)cc2)cc1 ; c1ccc(-c2nc(-c3ccccc3-c3ccccc3)nc(-n3c4ccccc4c4ccc5c(c6ccccc6n5-c5c(-c6ccccc6)ccc6c5c5ccccc5n6-c5ccccc5)c43)n2)cc1]

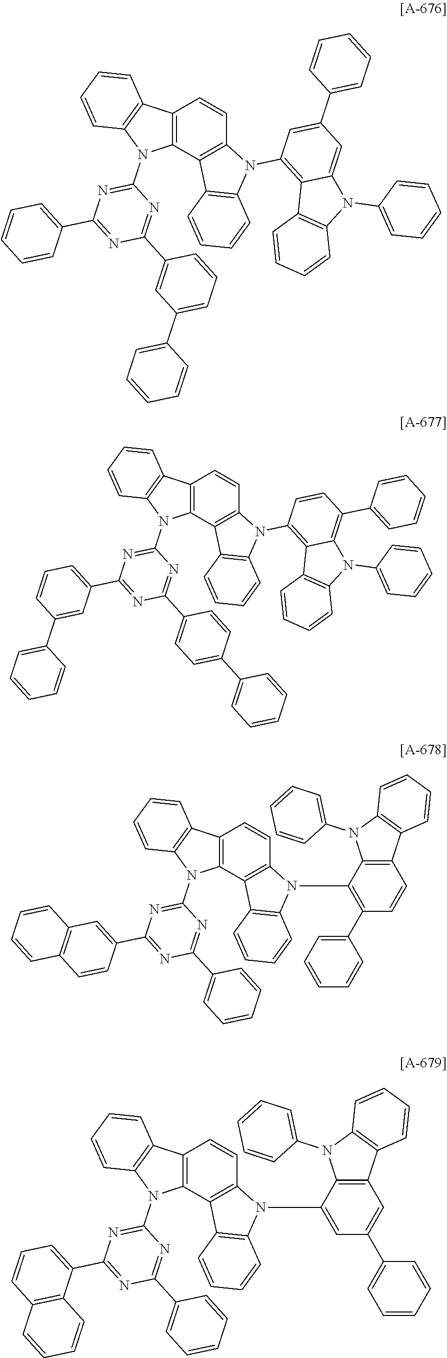 [SMILES: c1ccc(-c2cccc(-c3nc(-c4ccccc4)nc(-n4c5ccccc5c5ccc6c(c7ccccc7n6-c6cc(-c7ccccc7)cc7c6c6ccccc6n7-c6ccccc6)c54)n3)c2)cc1 ; c1ccc(-c2ccc(-c3nc(-c4cccc(-c5ccccc5)c4)nc(-n4c5ccccc5c5ccc6c(c7ccccc7n6-c6ccc(-c7ccccc7)c7c6c6ccccc6n7-c6ccccc6)c54)n3)cc2)cc1 ; c1ccc(-c2nc(-c3ccc4ccccc4c3)nc(-n3c4ccccc4c4ccc5c(c6ccccc6n5-c5c(-c6ccccc6)ccc6c7ccccc7n(-c7ccccc7)c56)c43)n2)cc1 ; c1ccc(-c2cc(-n3c4ccccc4c4c3ccc3c5ccccc5n(-c5nc(-c6ccccc6)nc(-c6cccc7ccccc67)n5)c34)c3c(c2)c2ccccc2n3-c2ccccc2)cc1]

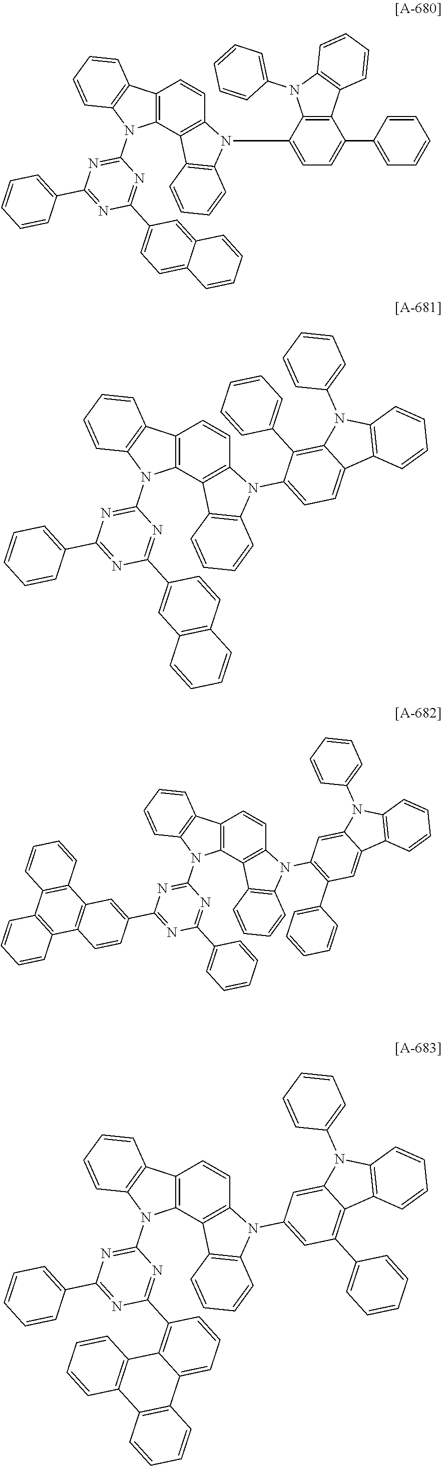 [SMILES: c1ccc(-c2nc(-c3ccc4ccccc4c3)nc(-n3c4ccccc4c4ccc5c(c6ccccc6n5-c5ccc(-c6ccccc6)c6c7ccccc7n(-c7ccccc7)c56)c43)n2)cc1 ; c1ccc(-c2nc(-c3ccc4ccccc4c3)nc(-n3c4ccccc4c4ccc5c(c6ccccc6n5-c5ccc6c7ccccc7n(-c7ccccc7)c6c5-c5ccccc5)c43)n2)cc1 ; c1ccc(-c2nc(-c3ccc4c5ccccc5c5ccccc5c4c3)nc(-n3c4ccccc4c4ccc5c(c6ccccc6n5-c5cc6c(cc5-c5ccccc5)c5ccccc5n6-c5ccccc5)c43)n2)cc1 ; c1ccc(-c2nc(-c3cccc4c5ccccc5c5ccccc5c34)nc(-n3c4ccccc4c4ccc5c(c6ccccc6n5-c5cc(-c6ccccc6)c6c7ccccc7n(-c7ccccc7)c6c5)c43)n2)cc1]

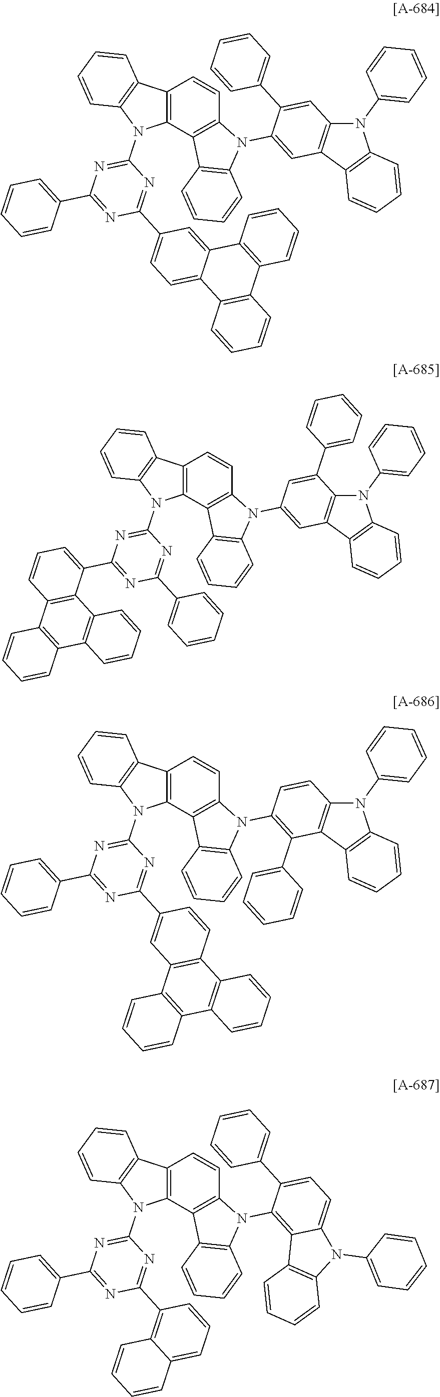 [SMILES: c1ccc(-c2nc(-c3ccc4c5ccccc5c5ccccc5c4c3)nc(-n3c4ccccc4c4ccc5c(c6ccccc6n5-c5cc6c7ccccc7n(-c7ccccc7)c6cc5-c5ccccc5)c43)n2)cc1 ; c1ccc(-c2nc(-c3cccc4c5ccccc5c5ccccc5c34)nc(-n3c4ccccc4c4ccc5c(c6ccccc6n5-c5cc(-c6ccccc6)c6c(c5)c5ccccc5n6-c5ccccc5)c43)n2)cc1 ; c1ccc(-c2nc(-c3ccc4c5ccccc5c5ccccc5c4c3)nc(-n3c4ccccc4c4ccc5c(c6ccccc6n5-c5ccc6c(c5-c5ccccc5)c5ccccc5n6-c5ccccc5)c43)n2)cc1 ; c1ccc(-c2nc(-c3cccc4ccccc34)nc(-n3c4ccccc4c4ccc5c(c6ccccc6n5-c5c(-c6ccccc6)ccc6c5c5ccccc5n6-c5ccccc5)c43)n2)cc1]

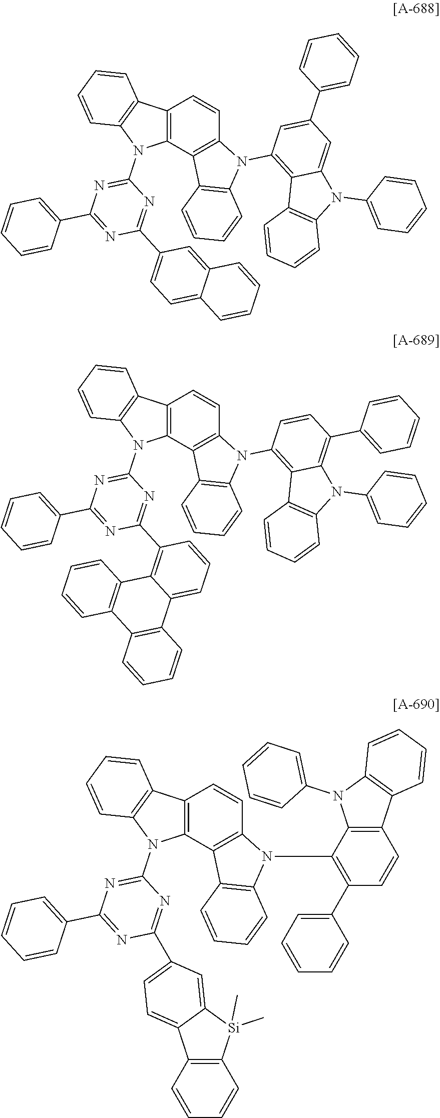 [SMILES: c1ccc(-c2cc(-n3c4ccccc4c4c3ccc3c5ccccc5n(-c5nc(-c6ccccc6)nc(-c6ccc7ccccc7c6)n5)c34)c3c4ccccc4n(-c4ccccc4)c3c2)cc1 ; c1ccc(-c2nc(-c3cccc4c5ccccc5c5ccccc5c34)nc(-n3c4ccccc4c4ccc5c(c6ccccc6n5-c5ccc(-c6ccccc6)c6c5c5ccccc5n6-c5ccccc5)c43)n2)cc1 ; C[Si]1(C)c2ccccc2-c2ccc(-c3nc(-c4ccccc4)nc(-n4c5ccccc5c5ccc6c(c7ccccc7n6-c6c(-c7ccccc7)ccc7c8ccccc8n(-c8ccccc8)c67)c54)n3)cc21]

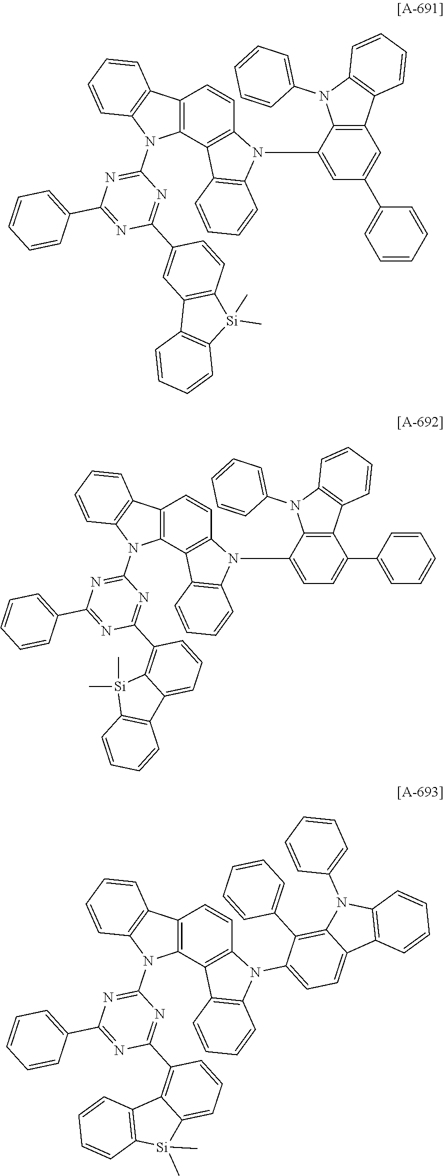 [SMILES: C[Si]1(C)c2ccccc2-c2cc(-c3nc(-c4ccccc4)nc(-n4c5ccccc5c5ccc6c(c7ccccc7n6-c6cc(-c7ccccc7)cc7c8ccccc8n(-c8ccccc8)c67)c54)n3)ccc21 ; C[Si]1(C)c2ccccc2-c2cccc(-c3nc(-c4ccccc4)nc(-n4c5ccccc5c5ccc6c(c7ccccc7n6-c6ccc(-c7ccccc7)c7c8ccccc8n(-c8ccccc8)c67)c54)n3)c21 ; C[Si]1(C)c2ccccc2-c2c(-c3nc(-c4ccccc4)nc(-n4c5ccccc5c5ccc6c(c7ccccc7n6-c6ccc7c8ccccc8n(-c8ccccc8)c7c6-c6ccccc6)c54)n3)cccc21]

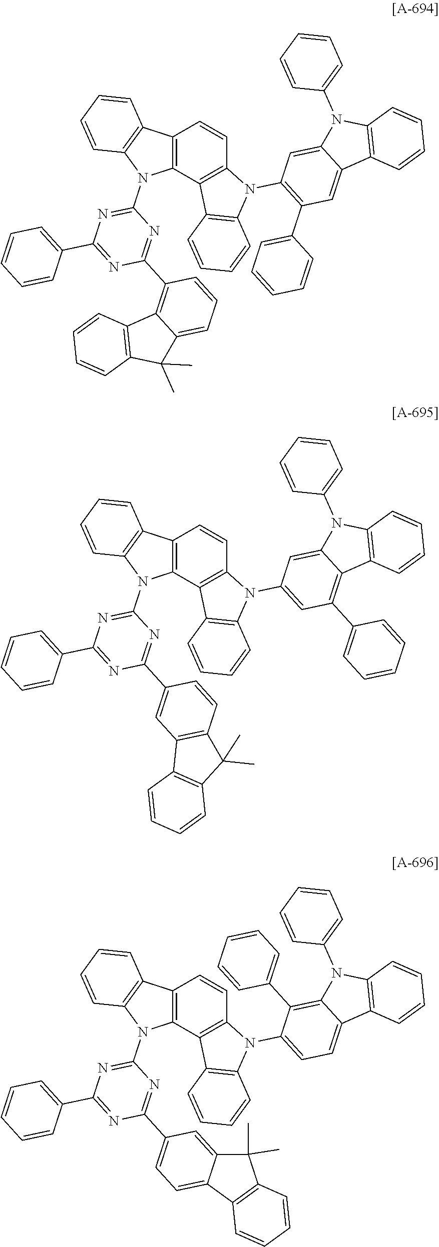 [SMILES: CC1(C)c2ccccc2-c2c(-c3nc(-c4ccccc4)nc(-n4c5ccccc5c5ccc6c(c7ccccc7n6-c6cc7c(cc6-c6ccccc6)c6ccccc6n7-c6ccccc6)c54)n3)cccc21 ; CC1(C)c2ccccc2-c2cc(-c3nc(-c4ccccc4)nc(-n4c5ccccc5c5ccc6c(c7ccccc7n6-c6cc(-c7ccccc7)c7c8ccccc8n(-c8ccccc8)c7c6)c54)n3)ccc21 ; CC1(C)c2ccccc2-c2ccc(-c3nc(-c4ccccc4)nc(-n4c5ccccc5c5ccc6c(c7ccccc7n6-c6ccc7c8ccccc8n(-c8ccccc8)c7c6-c6ccccc6)c54)n3)cc21]

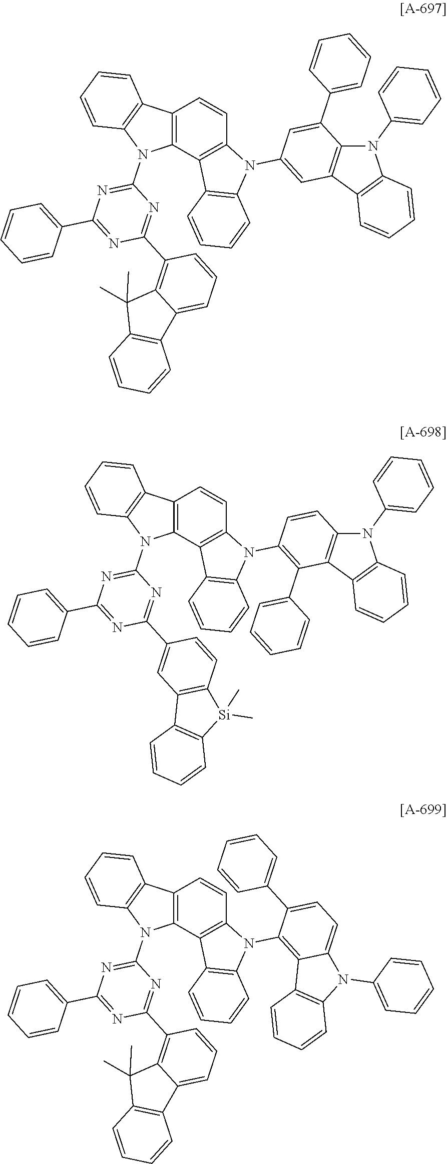 [SMILES: CC1(C)c2ccccc2-c2cccc(-c3nc(-c4ccccc4)nc(-n4c5ccccc5c5ccc6c(c7ccccc7n6-c6cc(-c7ccccc7)c7c(c6)c6ccccc6n7-c6ccccc6)c54)n3)c21 ; C[Si]1(C)c2ccccc2-c2cc(-c3nc(-c4ccccc4)nc(-n4c5ccccc5c5ccc6c(c7ccccc7n6-c6ccc7c(c6-c6ccccc6)c6ccccc6n7-c6ccccc6)c54)n3)ccc21 ; CC1(C)c2ccccc2-c2cccc(-c3nc(-c4ccccc4)nc(-n4c5ccccc5c5ccc6c(c7ccccc7n6-c6c(-c7ccccc7)ccc7c6c6ccccc6n7-c6ccccc6)c54)n3)c21]

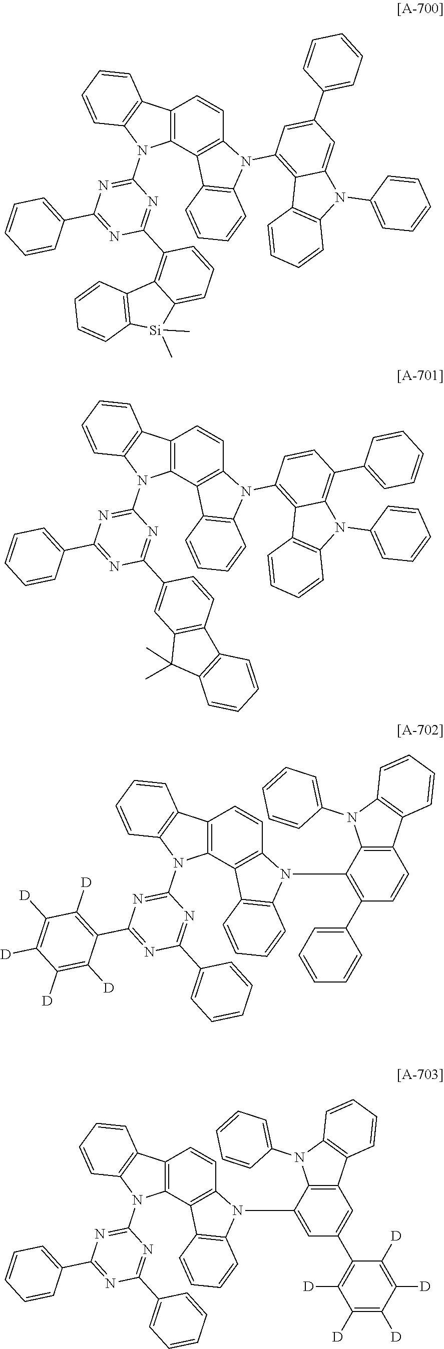

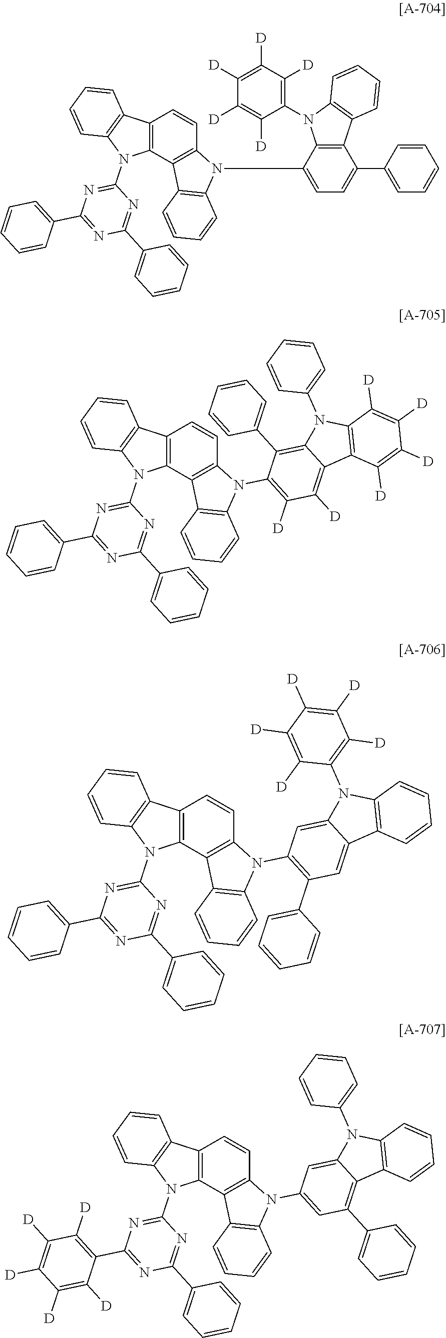

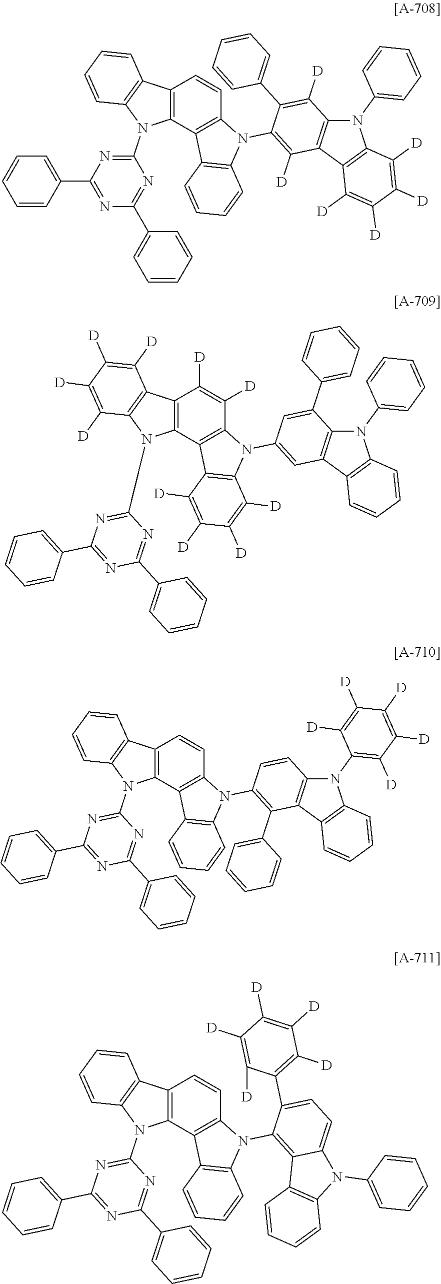

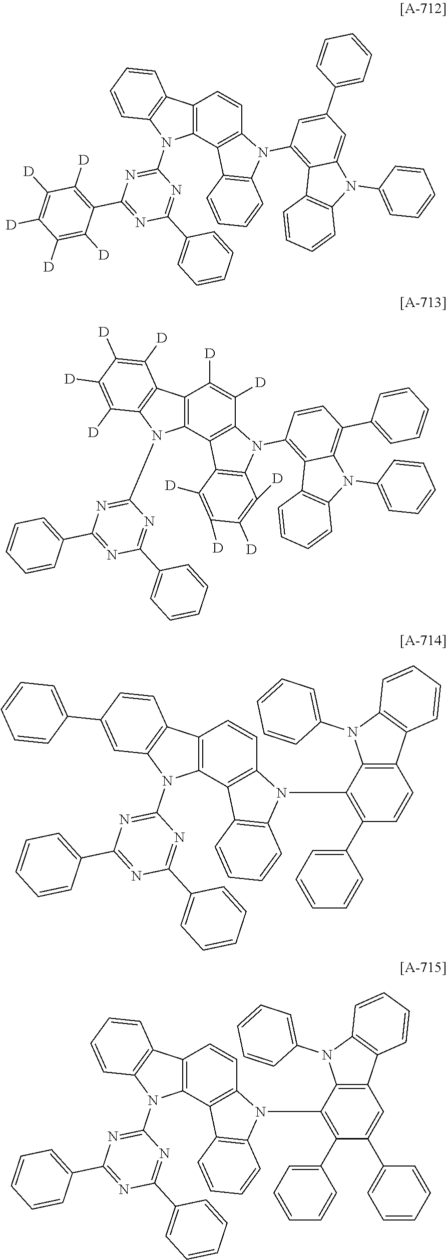

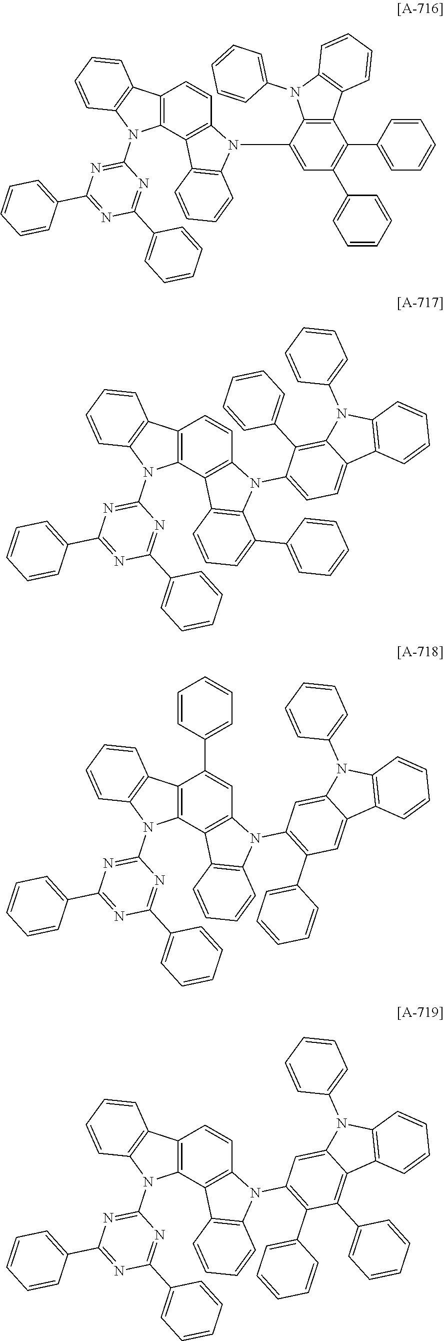 [SMILES: c1ccc(-c2nc(-c3ccccc3)nc(-n3c4ccccc4c4ccc5c(c6ccccc6n5-c5cc(-c6ccccc6)c(-c6ccccc6)c6c7ccccc7n(-c7ccccc7)c56)c43)n2)cc1 ; c1ccc(-c2nc(-c3ccccc3)nc(-n3c4ccccc4c4ccc5c(c6cccc(-c7ccccc7)c6n5-c5ccc6c7ccccc7n(-c7ccccc7)c6c5-c5ccccc5)c43)n2)cc1 ; c1ccc(-c2nc(-c3ccccc3)nc(-n3c4ccccc4c4c(-c5ccccc5)cc5c(c6ccccc6n5-c5cc6c(cc5-c5ccccc5)c5ccccc5n6-c5ccccc5)c43)n2)cc1 ; c1ccc(-c2nc(-c3ccccc3)nc(-n3c4ccccc4c4ccc5c(c6ccccc6n5-c5cc6c(c(-c7ccccc7)c5-c5ccccc5)c5ccccc5n6-c5ccccc5)c43)n2)cc1]

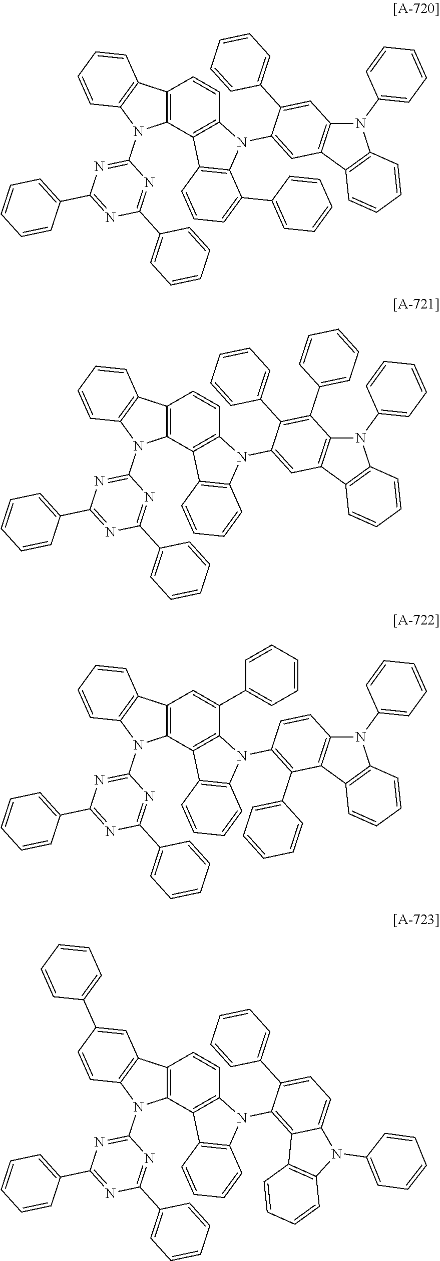 [SMILES: c1ccc(-c2nc(-c3ccccc3)nc(-n3c4ccccc4c4ccc5c(c6cccc(-c7ccccc7)c6n5-c5cc6c7ccccc7n(-c7ccccc7)c6cc5-c5ccccc5)c43)n2)cc1 ; c1ccc(-c2nc(-c3ccccc3)nc(-n3c4ccccc4c4ccc5c(c6ccccc6n5-c5cc6c7ccccc7n(-c7ccccc7)c6c(-c6ccccc6)c5-c5ccccc5)c43)n2)cc1 ; c1ccc(-c2nc(-c3ccccc3)nc(-n3c4ccccc4c4cc(-c5ccccc5)c5c(c6ccccc6n5-c5ccc6c(c5-c5ccccc5)c5ccccc5n6-c5ccccc5)c43)n2)cc1 ; c1ccc(-c2ccc3c(c2)c2ccc4c(c5ccccc5n4-c4c(-c5ccccc5)ccc5c4c4ccccc4n5-c4ccccc4)c2n3-c2nc(-c3ccccc3)nc(-c3ccccc3)n2)cc1]

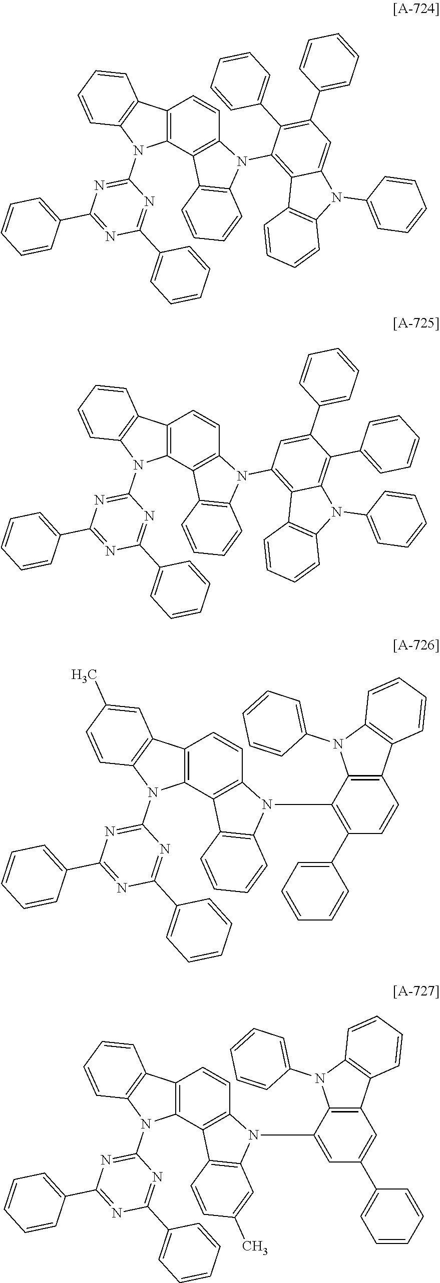 [SMILES: c1ccc(-c2nc(-c3ccccc3)nc(-n3c4ccccc4c4ccc5c(c6ccccc6n5-c5c(-c6ccccc6)c(-c6ccccc6)cc6c5c5ccccc5n6-c5ccccc5)c43)n2)cc1 ; c1ccc(-c2nc(-c3ccccc3)nc(-n3c4ccccc4c4ccc5c(c6ccccc6n5-c5cc(-c6ccccc6)c(-c6ccccc6)c6c5c5ccccc5n6-c5ccccc5)c43)n2)cc1 ; Cc1ccc2c(c1)c1ccc3c(c4ccccc4n3-c3c(-c4ccccc4)ccc4c5ccccc5n(-c5ccccc5)c34)c1n2-c1nc(-c2ccccc2)nc(-c2ccccc2)n1 ; Cc1ccc2c3c(ccc4c5ccccc5n(-c5nc(-c6ccccc6)nc(-c6ccccc6)n5)c43)n(-c3cc(-c4ccccc4)cc4c5ccccc5n(-c5ccccc5)c34)c2c1]

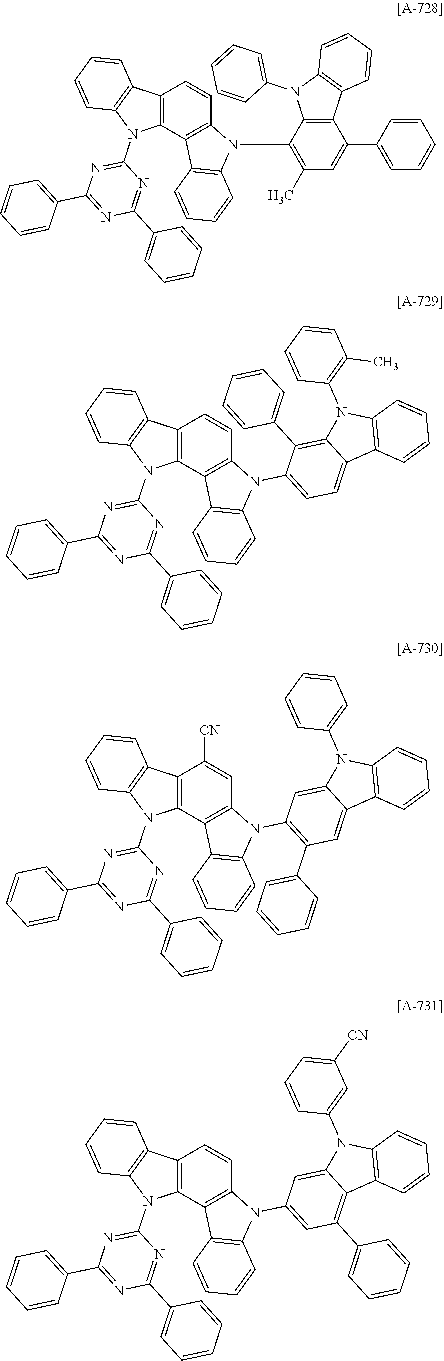 [SMILES: Cc1cc(-c2ccccc2)c2c3ccccc3n(-c3ccccc3)c2c1-n1c2ccccc2c2c1ccc1c3ccccc3n(-c3nc(-c4ccccc4)nc(-c4ccccc4)n3)c12 ; Cc1ccccc1-n1c2ccccc2c2ccc(-n3c4ccccc4c4c3ccc3c5ccccc5n(-c5nc(-c6ccccc6)nc(-c6ccccc6)n5)c34)c(-c3ccccc3)c21 ; N#Cc1cc2c(c3ccccc3n2-c2cc3c(cc2-c2ccccc2)c2ccccc2n3-c2ccccc2)c2c1c1ccccc1n2-c1nc(-c2ccccc2)nc(-c2ccccc2)n1 ; N#Cc1cccc(-n2c3ccccc3c3c(-c4ccccc4)cc(-n4c5ccccc5c5c4ccc4c6ccccc6n(-c6nc(-c7ccccc7)nc(-c7ccccc7)n6)c45)cc32)c1]

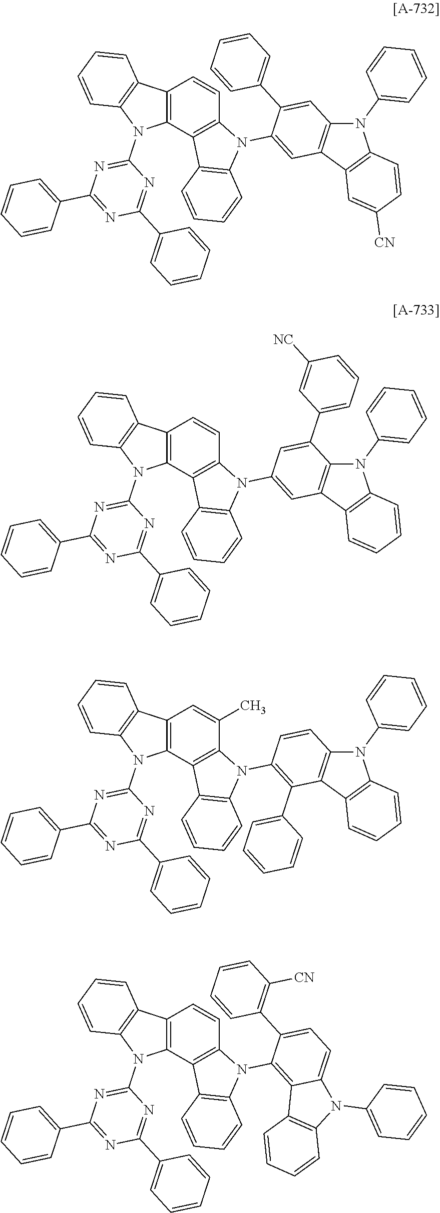 [SMILES: N#Cc1ccc2c(c1)c1cc(-n3c4ccccc4c4c3ccc3c5ccccc5n(-c5nc(-c6ccccc6)nc(-c6ccccc6)n5)c34)c(-c3ccccc3)cc1n2-c1ccccc1 ; N#Cc1cccc(-c2cc(-n3c4ccccc4c4c3ccc3c5ccccc5n(-c5nc(-c6ccccc6)nc(-c6ccccc6)n5)c34)cc3c4ccccc4n(-c4ccccc4)c23)c1 ; Cc1cc2c3ccccc3n(-c3nc(-c4ccccc4)nc(-c4ccccc4)n3)c2c2c3ccccc3n(-c3ccc4c(c3-c3ccccc3)c3ccccc3n4-c3ccccc3)c12 ; N#Cc1ccccc1-c1ccc2c(c1-n1c3ccccc3c3c1ccc1c4ccccc4n(-c4nc(-c5ccccc5)nc(-c5ccccc5)n4)c13)c1ccccc1n2-c1ccccc1]

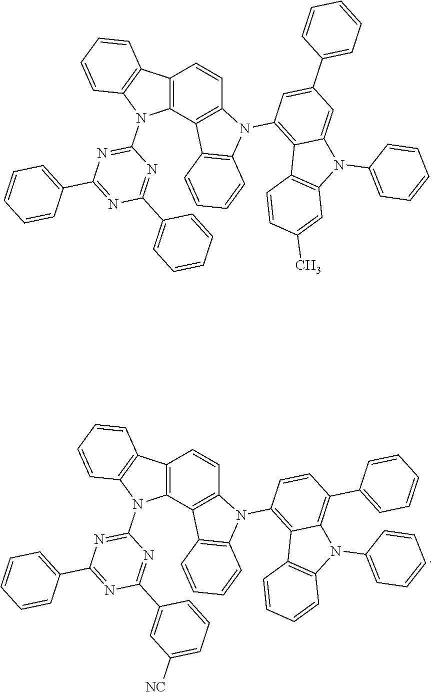 [SMILES: Cc1ccc2c3c(-n4c5ccccc5c5c4ccc4c6ccccc6n(-c6nc(-c7ccccc7)nc(-c7ccccc7)n6)c45)cc(-c4ccccc4)cc3n(-c3ccccc3)c2c1 ; N#Cc1cccc(-c2nc(-c3ccccc3)nc(-n3c4ccccc4c4ccc5c(c6ccccc6n5-c5ccc(-c6ccccc6)c6c5c5ccccc5n6-c5ccccc5)c43)n2)c1]

9. A composition for an organic optoelectronic device, the composition comprising:
a first compound; and
a second compound,
wherein:
the first compound is the compound for an organic optoelectronic device as claimed in claim 1, and
the second compound is:
a compound for an organic optoelectronic device represented by Chemical Formula 3; or
a compound for an organic optoelectronic device represented by a combination of Chemical Formula 4 and Chemical Formula 5: 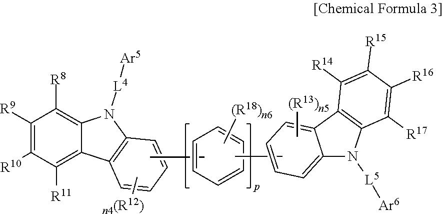
in Chemical Formula 3,
Ar⁵ and Ar⁶ are each independently a substituted or unsubstituted C6 to C20 aryl group or a substituted or unsubstituted C2 to C30 heterocyclic group,
L⁴ and L⁵ are each independently a single bond or a substituted or unsubstituted C6 to C20 arylene group,
R⁸ to R¹⁸ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
n4 and n5 are each independently an integer of 1 to 3,
n6 is an integer of 1 to 4, and
p is an integer of 0 to 2; 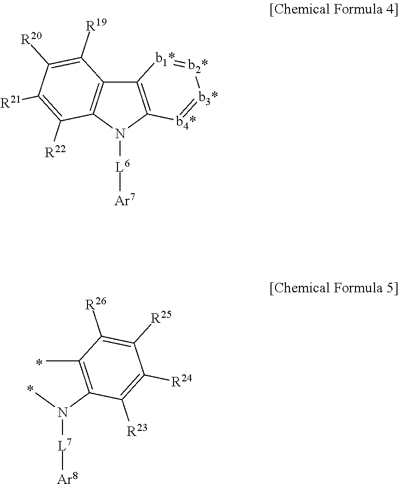
in Chemical Formulas 4 and 5,
Ar⁷ and Ar⁸ are each independently a substituted or unsubstituted C6 to C20 aryl group or a substituted or unsubstituted C2 to C30 heterocyclic group,
two adjacent ones of b1* to b4* of Chemical Formula 4 are linking carbons linked at * of Chemical Formula 5,
the remaining two of b1* to b4* of Chemical Formula 4, not linked at * of Chemical Formula 5 are each independently C-L^{b}-R^{b},
L^{b}, L⁶, and L⁷ are each independently a single bond or a substituted or unsubstituted C6 to C20 arylene group, and
R^{b} and R¹⁹ to R²⁶ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

10. The composition for an organic optoelectronic device as claimed in claim 9, wherein:
the second compound is represented by Chemical Formula 3,
Chemical Formula 3 is represented by Chemical Formula 3-8: 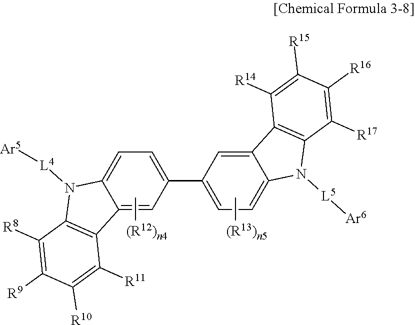
in Chemical Formula 3-8,
n4 and n5 are each independently an integer of 1 to 3,
R⁸ to R¹⁷ are each independently hydrogen, deuterium, or a substituted or unsubstituted C6 to C12 aryl group, and
moieties *-L⁴-Ar⁵ and *-L⁵-Ar⁶ are each independently a moiety of Group II, 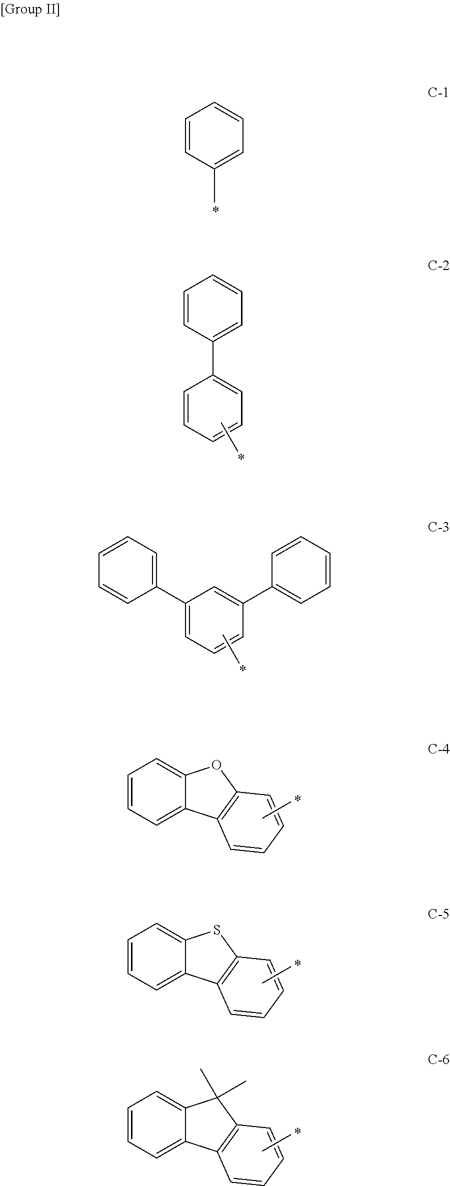 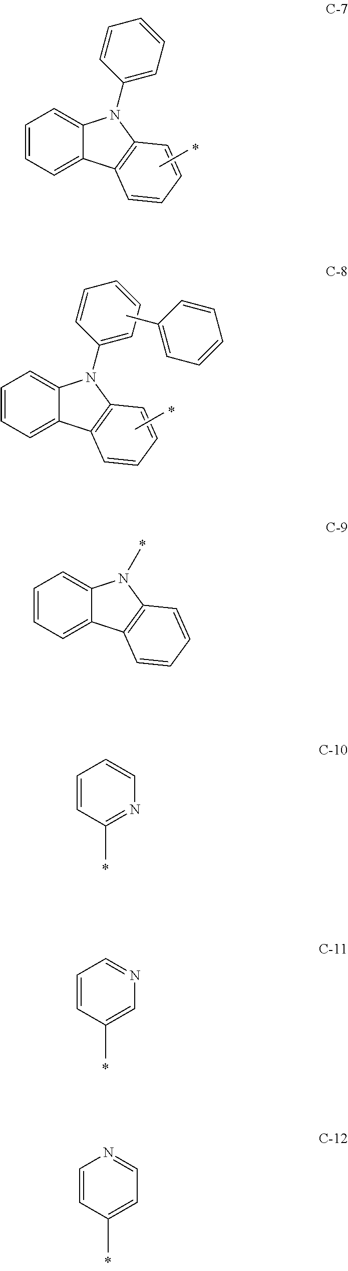 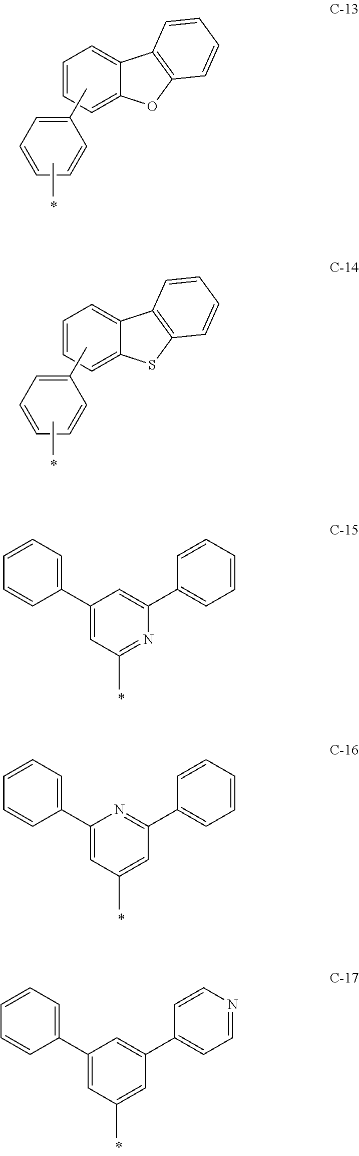 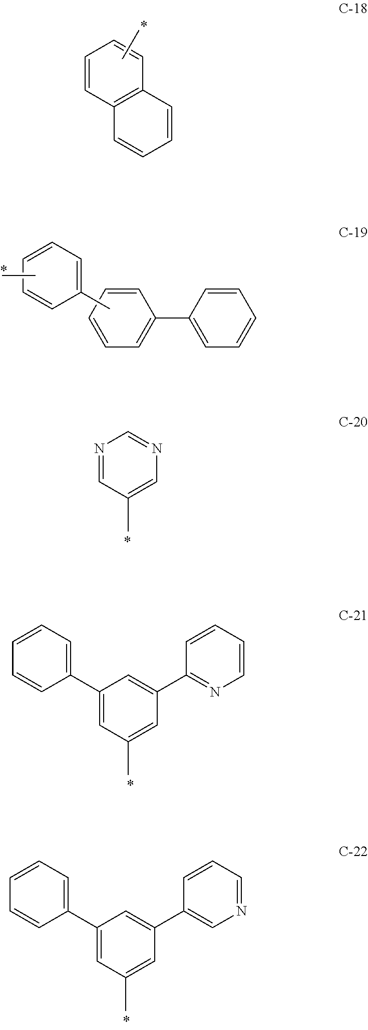 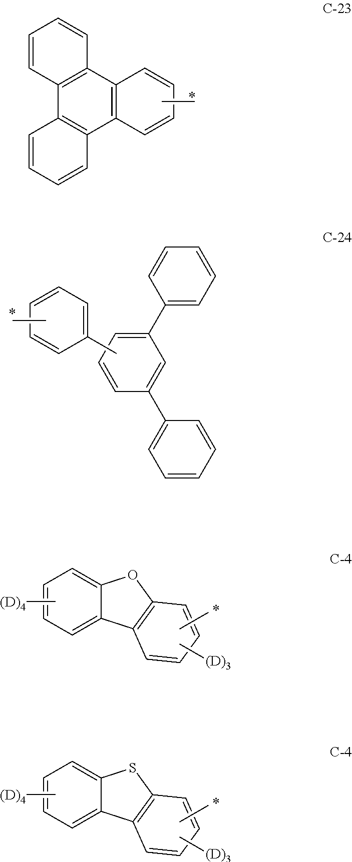 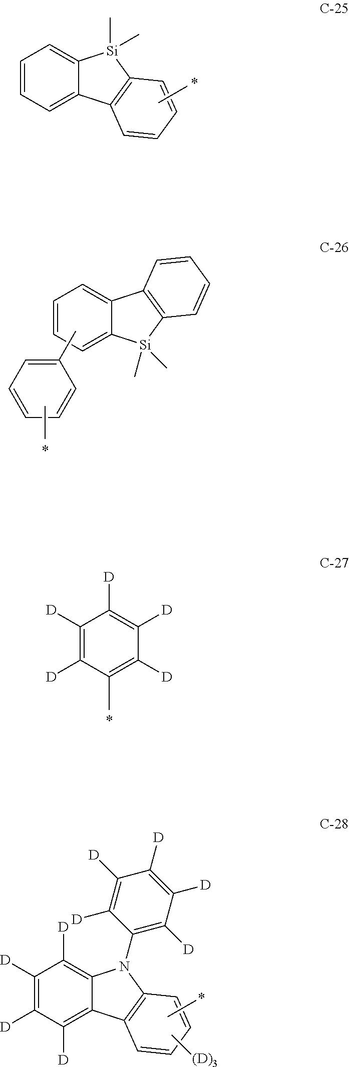 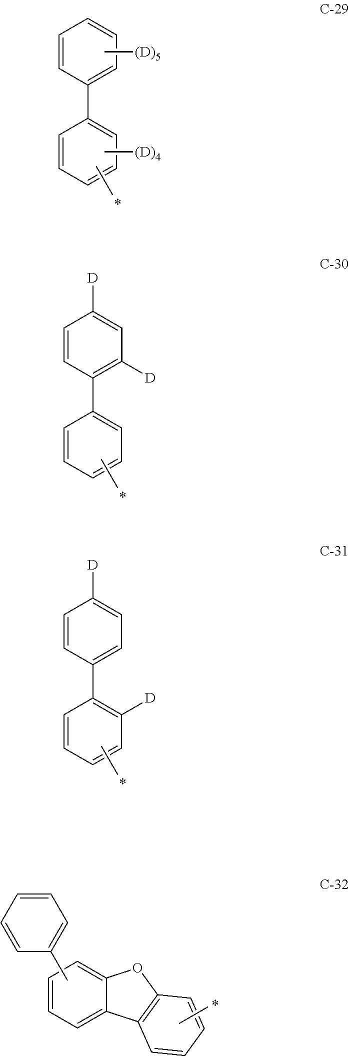 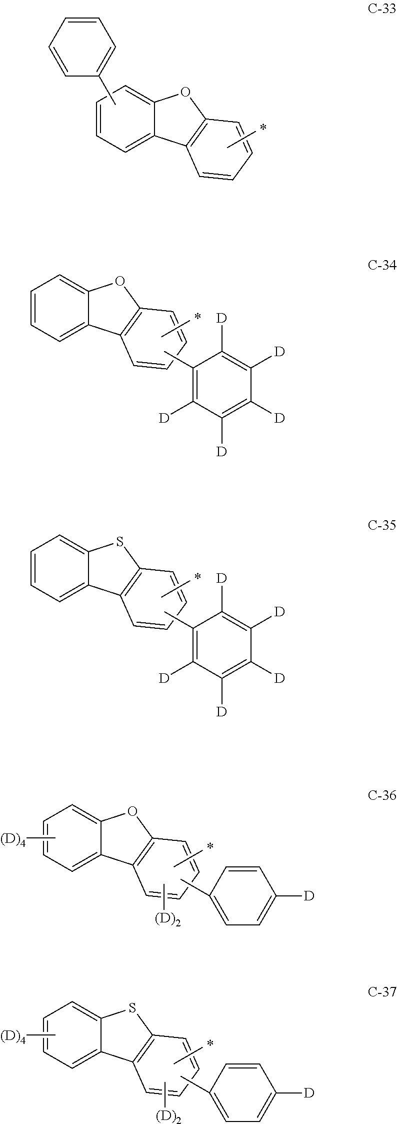
in Group II, * is a linking point.

11. The composition for an organic optoelectronic device as claimed in claim 9, wherein:
the second compound is represented by a combination of Chemical Formula 4 and Chemical Formula 5,
the combination of Chemical Formula 4 and Chemical Formula 5 is represented by Chemical Formula 4C: 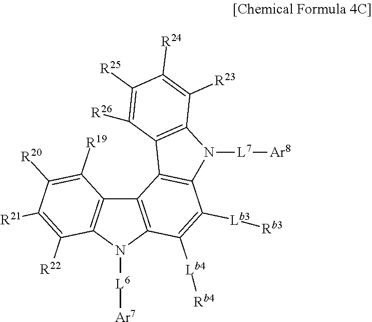
in Chemical Formula 4C,
L^{b3} and L^{b4} are each a single bond,
L⁶ and L⁷ are each independently a single bond or a substituted or unsubstituted C6 to C12 arylene group,
R¹⁹ to R²⁶, R^{b3} and R^{b4} are each independently hydrogen or a substituted or unsubstituted C6 to C12 aryl group, and
Ar⁷ and Ar⁸ are each independently a substituted or unsubstituted phenyl group, a substituted or unsubstituted naphthyl group or a substituted or unsubstituted biphenyl group.

12. An organic optoelectronic device, comprising:
an anode and a cathode facing each other, and
at least one organic layer between the anode and the cathode,
wherein the at least one organic layer includes the compound for an organic optoelectronic device of claim 1.

13. The organic photoelectronic device as claimed in claim 12, wherein:
the at least one organic layer includes a light emitting layer, and
the light emitting layer includes the compound for an organic optoelectronic device.

14. A display device comprising the organic photoelectronic device as claimed in claim 12.

15. An organic optoelectronic device, comprising:
an anode and a cathode facing each other, and
at least one organic layer between the anode and the cathode,
wherein the at least one organic layer includes the composition for an organic optoelectronic device of claim 9.

16. The organic photoelectronic device as claimed in claim 15, wherein:
the at least one organic layer includes a light emitting layer, and
the light emitting layer includes the composition for an organic optoelectronic device.

17. A display device comprising the organic photoelectronic device as claimed in claim 15.
